(19) United States
(12) Patent Application Publication
(10) Pub. No.: US 2024/0076678 A1
(43) Pub. Date: Mar. 7, 2024

(54) COMPOSITIONS AND METHODS FOR EPIGENETIC EDITING

(71) Applicant: Chroma Medicine, Inc., Boston, MA (US)

(72) Inventors: Morgan Maeder, Waban, MA (US); Ari Friedland, Cambridge, MA (US); Samantha Linder, Salem, MA (US); Vic Myer, Arlington, MA (US)

(21) Appl. No.: 18/338,049

(22) Filed: Jun. 20, 2023

Related U.S. Application Data
(63) Continuation of application No. PCT/US2021/064913, filed on Dec. 22, 2021.
(60) Provisional application No. 63/129,283, filed on Dec. 22, 2020.
(60) Provisional application No. 63/280,452, filed on Nov. 17, 2021.

(51) Int. Cl. C12N 15/113; C07K 14/47; C12N 9/22

(52) U.S. Cl. CPC C12N 15/1138; C07K 14/4702; C12N 9/22; A61K 38/00; C07K 2319/80; C12N 2310/20; C12N 2800/80

(57) ABSTRACT

Disclosed herein are compositions and methods comprising epigenetic editors for epigenetic editing or cells, nucleic acids, and vectors comprising the same. Also disclosed are epigenetically modified chromosomes.

### CROSS REFERENCE

This application is a continuation of International Application No. PCT/US2021/064913, filed on Dec. 22, 2021, which claims the benefit of U.S. Provisional Application No. 63/129,283, filed Dec. 22, 2020, and U.S. Provisional Application No. 63/280,452, filed Nov. 17, 2021, which are each incorporated herein by reference in its entirety.

### SEQUENCE LISTING

The instant application contains a Sequence Listing which has been submitted electronically in XML format and is hereby incorporated by reference in its entirety. Said XML copy, created on Jul. 14, 2023, is named 59073_708_301_SL.xml and is 1,748,707 bytes in size.

### BACKGROUND

Genome editing has been considered a promising therapeutic approach for treatment of genetic disease for over a decade. However, manipulation on the DNA level remains risky given the potential for undesired double stranded breaks, heterogenous repair including large and small insertions and deletions at the intended site, and toxicity.

### SUMMARY

Provided herein are compositions for epigenetic modification related to epigenetic editors and methods of using the same to generate epigenetic modification in target genomes, including those in host cells and organisms, without introducing changes to genomic sequences.

Described herein is an epigenetic editor comprising a fusion protein, wherein the fusion protein comprises (a) a first DNMT domain; (b) a DNA binding domain; (c) a first repressor domain; and (d) a second repressor domain. In some embodiments, the DNA binding domain binds to a target sequence in a target chromosome comprising a target gene. In some embodiments, the repressor domain specifically binds to an epigenetic effector protein in a cell comprising a target gene and directs the epigenetic editor to the target gene to effect an epigenetic modification in a nucleotide in the target gene or a histone bound to the target gene.

In some embodiments, the fusion protein further comprises a second DNMT domain. In some embodiments, the first DNMT domain is selected from the group consisting of a DNMT3A domain, a DNMT3B domain, a DNMT3C domain, and a DNMT3L domain. In some embodiments, the first DNMT domain is the DNMT3A domain. In some embodiments, the first DNMT domain is the DNMT3L domain. In some embodiments, the first DNMT domain is a human DNMT domain. In some embodiments, the human DNMT domain is a human DNMT3A domain. In some embodiments, the human DNMT domain is a human DNMT3L domain. In some embodiments, wherein the first DNMT domain is a mouse DNMT domain. In some embodiments, the mouse DNMT domain is a mouse DNMT3A domain. In some embodiments, the mouse DNMT domain is a mouse DNMT3L domain. In some embodiments, the first DNMT domain is a DNMT3A domain and the second DNMT domain is a DNMT3L domain. In some embodiments, the first DNMT domain is a human DNMT3A domain and the second DNMT domain is a human DNMT3L domain. In some embodiments, the first DNMT domain is a human DNMT3A domain and the second DNMT domain is a mouse DNMT3L domain. In some embodiments, the first DNMT domain is a mouse DNMT3A domain and the second DNMT domain is a human DNMT3L domain. In some embodiments, is a mouse DNMT3A domain and the second DNMT domain is a mouse DNMT3L domain.

In some embodiments, the first DNMT domain is a catalytic portion of a DNMT domain. In some embodiments, the second DNMT domain is a catalytic portion of a DNMT domain. In some embodiments, the first DNMT domain and the second DNMT domain are selected from the group consisting of SEQ ID NO: 32-66.

In some embodiments, at least one of the repressor domains is selected from the group consisting of: ZIM3, ZNF436, ZNF257, ZNF675, ZNF490, ZNF320, ZNF331, ZNF816, ZNF680, ZNF41, ZNF189, ZNF528, ZNF543, ZNF554, ZNF140, ZNF610, ZNF264, ZNF350, ZNF8, ZNF582, ZNF30, ZNF324, ZNF98, ZNF669, ZNF677, ZNF596, ZNF214, ZNF37A, ZNF34, ZNF250, ZNF547, ZNF273, ZNF354A, ZFP82, ZNF224, ZNF33A, ZNF45, ZNF175, ZNF595, ZNF184, ZNF419, ZFP28-1, ZFP28-2, ZNF18, ZNF213, ZNF394, ZFP1, ZFP14, ZNF416, ZNF557, ZNF566, ZNF729, ZIM2, ZNF254, ZNF764, ZNF785, ZNF10, CBX5, RYBP, YAF2, MGA, CBX1, SCMH1, MPP8, SUMO3, HERC2, BIN1, PCGF2, TOX, FOXA1, FOXA2, IRF2BP1, IRF2BP2, IRF2BPL IRF-2BP1_2 N-terminal domain, HOXA13, HOXB13, HOXC13, HOXA11, HOXC11, HOXC10, HOXA10, HOXB9, HOXA9, ZFP28, ZN334, ZN568, ZN37A, ZN181, ZN510, ZN862, ZN140, ZN208, ZN248, ZN571, ZN699, ZN726, ZIK1, ZNF2, Z705F, ZNF14, ZN471, ZN624, ZNF84, ZNF7, ZN891, ZN337, Z705G, ZN529, ZN729, ZN419, Z705A, ZNF45, ZN302, ZN486, ZN621, ZN688, ZN33A, ZN554, ZN878, ZN772, ZN224, ZN184, ZN544, ZNF57, ZN283, ZN549, ZN211, ZN615, ZN253, ZN226, ZN730, Z585A, ZN732, ZN681, ZN667, ZN649, ZN470, ZN484, ZN431, ZN382, ZN254, ZN124, ZN607, ZN317, ZN620, ZN141, ZN584, ZN540, ZN75D, ZN555, ZN658, ZN684, RBAK, ZN829, ZN582, ZN112, ZN716, HKR1, ZN350, ZN480, ZN416, ZNF92, ZN100, ZN736, ZNF74, CBX1, ZN443, ZN195, ZN530, ZN782, ZN791, ZN331, Z354C, ZN157, ZN727, ZN550, ZN793, ZN235, ZNF8, ZN724, ZN573, ZN577, ZN789, ZN718, ZN300, ZN383, ZN429, ZN677, ZN850, ZN454, ZN257, ZN264, ZFP82, ZFP14, ZN485, ZN737, ZNF44, ZN596, ZN565, ZN543, ZFP69, SUMO1, ZNF12, ZN169, ZN433, SUMO3, ZNF98, ZN175, ZN347, ZNF25, ZN519, Z585B, ZIM3, ZN517, ZN846, ZN230, ZNF66, ZFP1, ZN713, ZN816, ZN426, ZN674, ZN627, ZNF20, Z587B, ZN316, ZN233, ZN611, ZN556, ZN234, ZN560, ZNF77, ZN682, ZN614, ZN785, ZN445, ZFP30, ZN225, ZN551, ZN610, ZN528, ZN284, ZN418, MPP8, ZN490, ZN805, Z780B, ZN763, ZN285, ZNF85, ZN223, ZNF90, ZN557, ZN425, ZN229, ZN606, ZN155, ZN222, ZN442, ZNF91, ZN135, ZN778, RYBP, ZN534, ZN586, ZN567, ZN440, ZN583, ZN441, ZNF43, CBX5, ZN589, ZNF10, ZN563, ZN561, ZN136, ZN630, ZN527, ZN333, Z324B, ZN786, ZN709, ZN792, ZN599, ZN613, ZF69B, ZN799, ZN569, ZN564, ZN546, ZFP92, YAF2, ZN723, ZNF34, ZN439, ZFP57, ZNF19, ZN404, ZN274, CBX3, ZNF30, ZN250, ZN570, ZN675, ZN695, ZN548, ZN132, ZN738, ZN420, ZN626, ZN559, ZN460, ZN268, ZN304, ZIM2, ZN605, ZN844, SUMO5, ZN101, ZN783, ZN417, ZN182, ZN823, ZN177, ZN197, ZN717, ZN669, ZN256, ZN251, CBX4, PCGF2, CDY2, CDYL2, HERC2, ZN562, ZN461, Z324A, ZN766, ID2, TOX, ZN274, SCMH1, ZN214, CBX7, ID1, CREM, SCX, ASCL1, ZN764, SCML2, TWST1, CREB1, TERF1, ID3, CBX8, CBX4, GSX1, NKX22, ATF1, TWST2, ZNF17, TOX3, TOX4, ZMYM3, I2BP1, RHXF1, SSX2, I2BPL, ZN680, CBX1, TR168, HXA13, PHC3, TCF24, CBX3, HXB13, HEY1, PHC2, ZNF81, FIGLA, SAM11, KMT2B, HEY2, JDP2, HXC13, ASCL4, HHEX, HERC2, GSX2, BIN1, ETV7, ASCL3, PHC1, OTP, I2BP2, VGLL2, HXA11, PDLI4, ASCL2, CDX4, ZN860, LMBL4, PDIP3, NKX25, CEBPB, ISL1, CDX2, PROP1, SIN3B, SMBT1, HXC11, HXC10, PRS6A, VSX1, NKX23, MTG16, HMX3, HMX1, KIF22, CSTF2, CEBPE, DLX2, ZMYM3, PPARG, PRIC1, UNC4, BARX2, ALX3, TCF15, TERA, VSX2, HXD12, CDX1, TCF23, ALX1, HXA10, RX, CXXC5, SCML1, NFIL3, DLX6, MTG8, CBX8, CEBPD, SEC13, FIP1, ALX4, LHX3, PRIC2, MAGI3, NELL1, PRRX1, MTG8R, RAX2, DLX3, DLX1, NKX26, NAB1, SAMD7, PITX3, WDR5, MEOX2, NAB2, DHX8, FOXA2, CBX6, EMX2, CPSF6, HXC12, KDM4B, LMBL3, PHX2A, EMX1, NC2B, DLX4, SRY, ZN777, NELL1, ZN398, GATA3, BSH, SF3B4, TEAD1, TEAD3, RGAP1, PHF1, FOXA1, GATA2, FOXO3, ZN212, IRX4, ZBED6, LHX4, SIN3A, RBBP7, NKX61, TRI68, R51A1, MB3L1, DLX5, NOTC1, TERF2, ZN282, RGS12, ZN840, SPI2B, PAX7, NKX62, ASXL2, FOXO1, GATA3, GATA1, ZMYM5, ZN783, SPI2B, LRP1, MIXL1, SGT1, LMCD1, CEBPA, GATA2, SOX14, WTIP, PRP19, CBX6, NKX11, RBBP4, DMRT2, SMCA2, and fragments thereof. In some embodiments, at least one of the repressor domains is selected from the group consisting of: SEQ ID NO: 67-595. In some embodiments, at least one of the repressor domains is selected from the group consisting of: ZIM3, ZNF264, ZN577, ZN793, ZFP28, ZN627, RYBP, TOX, TOX3, TOX4, I2BP1, SCMH1, SCML2, CDYL2, CBX8, CBX5, and CBX1, and fragments thereof.

In some embodiments, one of the repressor domains is a KRAB domain. In some embodiments, the KRAB domain is a KOX1 KRAB domain.

In some embodiments, the DNA binding domain comprises a zinc finger motif. In some embodiments, the DNA binding domain comprises a zinc finger array. In some embodiments, the DNA binding domain comprises a nucleic acid guided DNA binding domain bound to a guide polynucleotide. In some embodiments, the DNA binding domain comprises CRISPR-Cas protein bound to the guide polynucleotide. In some embodiments, the guide polynucleotide hybridizes with a target sequence. In some embodiments, the CRISPR-Cas protein comprises a nuclease inactive Cas9 (dCas9). In some embodiments, the dCas9 is a dSpCas9. In some embodiments, the dSpCas9 is defined as SEQ ID NO: 3. In some embodiments, the CRISPR-Cas protein comprises a nuclease inactive Cas12a (dCas12a). In some embodiments, the CRISPR-Cas protein comprises a nuclease inactive CasX (dCasX).

In some embodiments, the fusion protein comprises from N-terminus to C-terminus: DNMT3A-DNMT3L-dSpCas9-KOX1KRAB—the second repressor domain. In some embodiments, a linker connects the domains of the fusion protein. In some embodiments, the linker is an XTEN linker. In some embodiments, the XTEN linker is selected from the group consisting of: XTEN-16, XTEN-18, and XTEN-80. In some embodiments, the fusion protein comprises from N-terminus to C-terminus: DNMT3A-DNMT3L-XTEN80-dSpCas9-XTEN16-KOX1KRAB-XTEN18—the second repressor domain.

Also described herein is an epigenetic editor comprising a fusion protein, wherein the fusion protein comprises (a) a first DNMT domain; (b) a DNA binding domain; and (c) a repressor domain, wherein the repressor domain is selected from the group consisting of: ZIM3, ZNF436, ZNF257, ZNF675, ZNF490, ZNF320, ZNF331, ZNF816, ZNF680, ZNF41, ZNF189, ZNF528, ZNF543, ZNF554, ZNF140, ZNF610, ZNF264, ZNF350, ZNF8, ZNF582, ZNF30, ZNF324, ZNF98, ZNF669, ZNF677, ZNF596, ZNF214, ZNF37A, ZNF34, ZNF250, ZNF547, ZNF273, ZNF354A, ZFP82, ZNF224, ZNF33A, ZNF45, ZNF175, ZNF595, ZNF184, ZNF419, ZFP28-1, ZFP28-2, ZNF18, ZNF213, ZNF394, ZFP1, ZFP14, ZNF416, ZNF557, ZNF566, ZNF729, ZIM2, ZNF254, ZNF764, ZNF785, ZNF10, CBX5, RYBP, YAF2, MGA, CBX1, SCMH1, MPP8, SUMO3, HERC2, BIN1, PCGF2, TOX, FOXA1, FOXA2, IRF2BP1, IRF2BP2, IRF2BPL IRF-2BP1_2 N-terminal domain, HOXA13, HOXB13, HOXC13, HOXA11, HOXC11, HOXC10, HOXA10, HOXB9, HOXA9, ZFP28, ZN334, ZN568, ZN37A, ZN181, ZN510, ZN862, ZN140, ZN208, ZN248, ZN571, ZN699, ZN726, ZIK1, ZNF2, Z705F, ZNF14, ZN471, ZN624, ZNF84, ZNF7, ZN891, ZN337, Z705G, ZN529, ZN729, ZN419, Z705A, ZNF45, ZN302, ZN486, ZN621, ZN688, ZN33A, ZN554, ZN878, ZN772, ZN224, ZN184, ZN544, ZNF57, ZN283, ZN549, ZN211, ZN615, ZN253, ZN226, ZN730, Z585A, ZN732, ZN681, ZN667, ZN649, ZN470, ZN484, ZN431, ZN382, ZN254, ZN124, ZN607, ZN317, ZN620, ZN141, ZN584, ZN540, ZN75D, ZN555, ZN658, ZN684, RBAK, ZN829, ZN582, ZN112, ZN716, HKR1, ZN350, ZN480, ZN416, ZNF92, ZN100, ZN736, ZNF74, CBX1, ZN443, ZN195, ZN530, ZN782, ZN791, ZN331, Z354C, ZN157, ZN727, ZN550, ZN793, ZN235, ZNF8, ZN724, ZN573, ZN577, ZN789, ZN718, ZN300, ZN383, ZN429, ZN677, ZN850, ZN454, ZN257, ZN264, ZFP82, ZFP14, ZN485, ZN737, ZNF44, ZN596, ZN565, ZN543, ZFP69, SUMO1, ZNF12, ZN169, ZN433, SUMO3, ZNF98, ZN175, ZN347, ZNF25, ZN519, Z585B, ZIM3, ZN517, ZN846, ZN230, ZNF66, ZFP1, ZN713, ZN816, ZN426, ZN674, ZN627, ZNF20, Z587B, ZN316, ZN233, ZN611, ZN556, ZN234, ZN560, ZNF77, ZN682, ZN614, ZN785, ZN445, ZFP30, ZN225, ZN551, ZN610, ZN528, ZN284, ZN418, MPP8, ZN490, ZN805, Z780B, ZN763, ZN285, ZNF85, ZN223, ZNF90, ZN557, ZN425, ZN229, ZN606, ZN155, ZN222, ZN442, ZNF91, ZN135, ZN778, RYBP, ZN534, ZN586, ZN567, ZN440, ZN583, ZN441, ZNF43, CBX5, ZN589, ZNF10, ZN563, ZN561, ZN136, ZN630, ZN527, ZN333, Z324B, ZN786, ZN709, ZN792, ZN599, ZN613, ZF69B, ZN799, ZN569, ZN564, ZN546, ZFP92, YAF2, ZN723, ZNF34, ZN439, ZFP57, ZNF19, ZN404, ZN274, CBX3, ZNF30, ZN250, ZN570, ZN675, ZN695, ZN548, ZN132, ZN738, ZN420, ZN626, ZN559, ZN460, ZN268, ZN304, ZIM2, ZN605, ZN844, SUMO5, ZN101, ZN783, ZN417, ZN182, ZN823, ZN177, ZN197, ZN717, ZN669, ZN256, ZN251, CBX4, PCGF2, CDY2, CDYL2, HERC2, ZN562, ZN461, Z324A, ZN766, ID2, TOX, ZN274, SCMH1, ZN214, CBX7, ID1, CREM, SCX, ASCL1, ZN764, SCML2, TWST1, CREB1, TERF1, ID3, CBX8, CBX4, GSX1, NKX22, ATF1, TWST2, ZNF17, TOX3, TOX4, ZMYM3, I2BP1, RHXF1, SSX2, I2BPL, ZN680, CBX1, TR168, HXA13, PHC3, TCF24, CBX3, HXB13, HEY1, PHC2, ZNF81, FIGLA, SAM11, KMT2B, HEY2, JDP2, HXC13, ASCL4, HHEX, HERC2, GSX2, BIN1, ETV7, ASCL3, PHC1, OTP, I2BP2, VGLL2, HXA11, PDLI4, ASCL2, CDX4, ZN860, LMBL4, PDIP3, NKX25, CEBPB, ISL1, CDX2, PROP1, SIN3B, SMBT1, HXC11, HXC10, PRS6A, VSX1, NKX23, MTG16, HMX3, HMX1, KIF22, CSTF2, CEBPE, DLX2, ZMYM3, PPARG, PRIC1, UNC4, BARX2, ALX3, TCF15, TERA, VSX2, HXD12, CDX1, TCF23, ALX1, HXA10, RX, CXXC5, SCML1, NFIL3, DLX6, MTG8, CBX8, CEBPD, SEC13, FIP1, ALX4, LHX3, PRIC2, MAGI3, NELL1, PRRX1, MTG8R, RAX2, DLX3, DLX1, NKX26, NAB1, SAMD7, PITX3, WDR5, MEOX2, NAB2, DHX8, FOXA2, CBX6, EMX2, CPSF6, HXC12, KDM4B, LMBL3, PHX2A, EMX1, NC2B, DLX4, SRY, ZN777, NELL1, ZN398, GATA3, BSH, SF3B4, TEAD1, TEAD3, RGAP1, PHF1, FOXA1, GATA2, FOXO3, ZN212, IRX4, ZBED6, LHX4, SIN3A, RBBP7, NKX61, TR168, R51A1, MB3L1, DLX5, NOTC1, TERF2, ZN282, RGS12, ZN840, SPI2B, PAX7, NKX62, ASXL2, FOXO1, GATA3, GATA1, ZMYM5, ZN783, SPI2B, LRP1, MIXL1, SGT1, LMCD1, CEBPA, GATA2, SOX14, WTIP, PRP19, CBX6, NKX11, RBBP4, DMRT2, SMCA2 and fragments thereof.

In some embodiments, at least one of the repressor domains is selected from the group consisting of: SEQ ID NO: 67-595. In some embodiments, the DNA binding domain binds to a target sequence in a target chromosome comprising a target gene. In some embodiments, the repressor domain specifically binds to an epigenetic effector protein in a cell comprising a target gene and directs the epigenetic editor to the target gene to effect an epigenetic modification in a nucleotide in the target gene or a histone bound to the target gene. In some embodiments, the repressor domains is selected from the group consisting of ZIM3, ZNF264, ZN577, ZN793, ZFP28, ZN627, RYBP, TOX, TOX3, TOX4, I2BP1, SCMH1, SCML2, CDYL2, CBX8, CBX5, and CBX1, and fragments thereof.

In some embodiments, the fusion protein further comprises a second DNMT domain. In some embodiments, the first DNMT domain is selected from the group consisting of a DNMT3A domain, a DNMT3B domain, a DNMT3C domain, and a DNMT3L domain. In some embodiments, the first DNMT domain is the DNMT3A domain. In some embodiments, the first DNMT domain is the DNMT3L domain. In some embodiments, the first DNMT domain is a human DNMT domain. In some embodiments, the first human DNMT domain is a human DNMT3A domain. In some embodiments, the human DNMT domain is a human DNMT3L domain. In some embodiments, the first DNMT domain is a mouse DNMT domain. In some embodiments, the mouse DNMT domain is a mouse DNMT3A domain. In some embodiments, the mouse DNMT domain is a mouse DNMT3L domain. In some embodiments, the first DNMT domain is a DNMT3A domain and the second DNMT domain is a DNMT3L domain. In some embodiments, the first DNMT domain is a human DNMT3A domain and the second DNMT domain is a human DNMT3L domain. In some embodiments, the first DNMT domain is a human DNMT3A domain and the second DNMT domain is a mouse DNMT3L domain. In some embodiments, the first DNMT domain is a mouse DNMT3A domain and the second DNMT domain is a human DNMT3L domain. In some embodiments, the first DNMT domain is a mouse DNMT3A domain and the second DNMT domain is a mouse DNMT3L domain. In some embodiments, the first DNMT domain is a catalytic portion of the DNMT domain. In some embodiments, the second DNMT domain is a catalytic portion of a DNMT domain. In some embodiments, the first DNMT domain and the second DNMT domain are selected from the group consisting of SEQ ID NO: 32-66.

In some embodiments, the DNA binding domain comprises a zinc finger motif. In some embodiments, the DNA binding domain comprises a zinc finger array. In some embodiments, the DNA binding domain comprises a nucleic acid guided DNA binding domain bound to a guide polynucleotide. In some embodiments, the DNA binding domain comprises CRISPR-Cas protein bound to the guide polynucleotide. In some embodiments, the guide polynucleotide hybridizes with a target sequence. In some embodiments, the CRISPR-Cas protein comprises a nuclease inactive Cas9 (dCas9). In some embodiments, the dCas9 is a dSpCas9. In some embodiments, the dSpCas9 is defined as SEQ ID NO: 3. In some embodiments, the CRISPR-Cas protein comprises a nuclease inactive Cas12a (dCas12a). In some embodiments, the CRISPR-Cas protein comprises a nuclease inactive CasX (dCasX).

In some embodiments, the fusion protein domain comprises from N-terminus to C-terminus DNMT3A-DNMT3L-dSpCas9—the repressor domain. In some embodiments, a linker connects the domains of the fusion protein. In some embodiments, the linker is an XTEN linker. In some embodiments, the XTEN linker is selected from the group consisting of: XTEN-16, XTEN-18, and XTEN-80. In some embodiments, the fusion protein comprises from N-terminus to C-terminus: DNMT3A-DNMT3L-XTEN80-dSpCas9-XTEN16—the repressor domain.

Also described herein is an epigenetic editor comprising a fusion protein, wherein the fusion protein comprises (a) a demethylase domain; (b) a DNA binding domain; and (c) an activator domain. In some embodiments, there is increased expression of the target gene when contacted with the epigenetic editor of any of the preceding claims as compared to the target gene not contacted with the epigenetic editor.

Also described herein is an epigenetic editor comprising a fusion protein, wherein the fusion protein comprises (a) a DNA binding domain; (b) a repressor domain; (c) a first catalytic domain wherein the catalytic domain is selected from the group consisting of a DNMT3A catalytic domain and a DNMT3L catalytic domain; and (d) a second catalytic domain wherein the catalytic domain is selected from the group consisting of a DNMT3A catalytic domain and a DNMT3L catalytic domain, wherein the first catalytic domain has less than 380 amino acids, or wherein the second catalytic domain has less than 380 amino acids.

Also described herein is a method for modifying an epigenetic state of a target gene in a target chromosome, the method comprising contacting the target chromosome with an epigenetic editor, wherein the epigenetic editor comprises (a) a first DNMT domain; (b) a DNA binding domain; (c) a first repressor domain; and (d) a second repressor domain, and wherein the DNA binding domain binds to a target sequence in the target chromosome and directs the epigenetic effector domain to effect a site-specific epigenetic modification in the target gene or a histone bound to the target gene in the target chromosome, thereby modifying the epigenetic state of the target gene.

Also described herein is a method for modulating expression of a target gene in a target chromosome, the method comprising contacting the target chromosome with an epigenetic editor, wherein the epigenetic editor comprises (a) a first DNMT domain; (b) a DNA binding domain; (c) a first repressor domain; and a second repressor domain, and wherein the DNA binding domain binds to a target sequence in the target chromosome and directs the epigenetic effector domain to effect a site-specific epigenetic modification in the target gene or a histone bound to the target gene in the target chromosome, thereby modulating the epigenetic state of the target gene.

Also described herein is a method for treating a disease in a subject in need thereof, the method comprising administering to the subject an epigenetic editor, wherein the epigenetic editor comprises (a) a first DNMT domain; (b) a DNA binding domain; (c) a first repressor domain; and (d) a second repressor domain, wherein the DNA binding domain binds to a target sequence in the target chromosome and directs the epigenetic effector domain to effect a site-specific epigenetic modification in the target gene or a histone bound to the target gene in the target chromosome, thereby treating the disease, wherein the target gene is associated with disease, and wherein the site-specific epigenetic modification modulates expression of the target gene, thereby treating the disease.

In some embodiments, the site-specific epigenetic modification is within 3000 base pairs upstream or downstream of the target sequence. In some embodiments, the site-specific epigenetic modification is within 2000 base pairs upstream or downstream of the target sequence. In some embodiments, the site-specific epigenetic modification is within 3000 base pairs upstream or downstream of an expression regulatory sequence. In some embodiments, the site-specific epigenetic modification is within 2000 base pairs upstream or downstream of the expression regulatory sequence. In some embodiments, the site-specific epigenetic modification is within 1000 base pairs upstream or downstream of the expression regulatory sequence.

In some embodiments, the method comprises administering to the subject a cell comprising the epigenetic editor. In some embodiments, the cell is an allogeneic cell. In some embodiments, the cell is an autologous cell. In some embodiments, the epigenetic modification is within a coding region of the target gene. In some embodiments, the target gene comprises an allele associated with a disease.

In some embodiments, the fusion protein further comprises a second DNMT domain. In some embodiments, the first DNMT domain is selected from the group consisting of a DNMT3A domain, a DNMT3B domain, a DNMT3C domain, and a DNMT3L domain. In some embodiments, the first DNMT domain is the DNMT3A domain. In some embodiments, the first DNMT domain is the DNMT3L domain. In some embodiments, the first DNMT domain is a human DNMT domain. In some embodiments, the human DNMT domain is a human DNMT3A domain. In some embodiments, the human DNMT domain is a human DNMT3L domain. In some embodiments, the first DNMT domain is a mouse DNMT domain. In some embodiments, the mouse DNMT domain is a mouse DNMT3A domain. In some embodiments, the mouse DNMT domain is a mouse DNMT3L domain. In some embodiments, the first DNMT domain is a DNMT3A domain and the second DNMT domain is a DNMT3L domain. In some embodiments, the first DNMT domain is a human DNMT3A domain and the second DNMT domain is a human DNMT3L domain. In some embodiments, the first DNMT domain is a human DNMT3A domain and the second DNMT domain is a mouse DNMT3L domain. In some embodiments, the first DNMT domain is the mouse DNMT3A domain and the second DNMT domain is a human DNMT3L domain. In some embodiments, the first DNMT domain is a mouse DNMT3A domain and the second DNMT domain is a mouse DNMT3L domain.

In some embodiments, the first DNMT domain is a catalytic portion of a DNMT domain. In some embodiments, the second DNMT domain is a catalytic portion of a DNMT domain. In some embodiments, the first DNMT domain and the second DNMT domain are selected from the group consisting of SEQ ID NO: 32-66.

In some embodiments, at least one of the repressor domains is selected from the group consisting of: ZIM3, ZNF436, ZNF257, ZNF675, ZNF490, ZNF320, ZNF331, ZNF816, ZNF680, ZNF41, ZNF189, ZNF528, ZNF543, ZNF554, ZNF140, ZNF610, ZNF264, ZNF350, ZNF8, ZNF582, ZNF30, ZNF324, ZNF98, ZNF669, ZNF677, ZNF596, ZNF214, ZNF37A, ZNF34, ZNF250, ZNF547, ZNF273, ZNF354A, ZFP82, ZNF224, ZNF33A, ZNF45, ZNF175, ZNF595, ZNF184, ZNF419, ZFP28-1, ZFP28-2, ZNF18, ZNF213, ZNF394, ZFP1, ZFP14, ZNF416, ZNF557, ZNF566, ZNF729, ZIM2, ZNF254, ZNF764, ZNF785, ZNF10, CBX5, RYBP, YAF2, MGA, CBX1, SCMH1, MPP8, SUMO3, HERC2, BIN1, PCGF2, TOX, FOXA1, FOXA2, IRF2BP1, IRF2BP2, IRF2BPL IRF-2BP1_2 N-terminal domain, HOXA13, HOXB13, HOXC13, HOXA11, HOXC11, HOXC10, HOXA10, HOXB9, HOXA9, ZFP28, ZN334, ZN568, ZN37A, ZN181, ZN510, ZN862, ZN140, ZN208, ZN248, ZN571, ZN699, ZN726, ZIK1, ZNF2, Z705F, ZNF14, ZN471, ZN624, ZNF84, ZNF7, ZN891, ZN337, Z705G, ZN529, ZN729, ZN419, Z705A, ZNF45, ZN302, ZN486, ZN621, ZN688, ZN33A, ZN554, ZN878, ZN772, ZN224, ZN184, ZN544, ZNF57, ZN283, ZN549, ZN211, ZN615, ZN253, ZN226, ZN730, Z585A, ZN732, ZN681, ZN667, ZN649, ZN470, ZN484, ZN431, ZN382, ZN254, ZN124, ZN607, ZN317, ZN620, ZN141, ZN584, ZN540, ZN75D, ZN555, ZN658, ZN684, RBAK, ZN829, ZN582, ZN112, ZN716, HKR1, ZN350, ZN480, ZN416, ZNF92, ZN100, ZN736, ZNF74, CBX1, ZN443, ZN195, ZN530, ZN782, ZN791, ZN331, Z354C, ZN157, ZN727, ZN550, ZN793, ZN235, ZNF8, ZN724, ZN573, ZN577, ZN789, ZN718, ZN300, ZN383, ZN429, ZN677, ZN850, ZN454, ZN257, ZN264, ZFP82, ZFP14, ZN485, ZN737, ZNF44, ZN596, ZN565, ZN543, ZFP69, SUMO1, ZNF12, ZN169, ZN433, SUMO3, ZNF98, ZN175, ZN347, ZNF25, ZN519, Z585B, ZIM3, ZN517, ZN846, ZN230, ZNF66, ZFP1, ZN713, ZN816, ZN426, ZN674, ZN627, ZNF20, Z587B, ZN316, ZN233, ZN611, ZN556, ZN234, ZN560, ZNF77, ZN682, ZN614, ZN785, ZN445, ZFP30, ZN225, ZN551, ZN610, ZN528, ZN284, ZN418, MPP8, ZN490, ZN805, Z780B, ZN763, ZN285, ZNF85, ZN223, ZNF90, ZN557, ZN425, ZN229, ZN606, ZN155, ZN222, ZN442, ZNF91, ZN135, ZN778, RYBP, ZN534, ZN586, ZN567, ZN440, ZN583, ZN441, ZNF43, CBX5, ZN589, ZNF10, ZN563, ZN561, ZN136, ZN630, ZN527, ZN333, Z324B, ZN786, ZN709, ZN792, ZN599, ZN613, ZF69B, ZN799, ZN569, ZN564, ZN546, ZFP92, YAF2, ZN723, ZNF34, ZN439, ZFP57, ZNF19, ZN404, ZN274, CBX3, ZNF30, ZN250, ZN570, ZN675, ZN695, ZN548, ZN132, ZN738, ZN420, ZN626, ZN559, ZN460, ZN268, ZN304, ZIM2, ZN605, ZN844, SUMO5, ZN101, ZN783, ZN417, ZN182, ZN823, ZN177, ZN197, ZN717, ZN669, ZN256, ZN251, CBX4, PCGF2, CDY2, CDYL2, HERC2, ZN562, ZN461, Z324A, ZN766, ID2, TOX, ZN274, SCMH1, ZN214, CBX7, ID1, CREM, SCX, ASCL1, ZN764, SCML2, TWST1, CREB1, TERF1, ID3, CBX8, CBX4, GSX1, NKX22, ATF1, TWST2, ZNF17, TOX3, TOX4, ZMYM3, I2BP1, RHXF1, SSX2, I2BPL, ZN680, CBX1, TR168, HXA13, PHC3, TCF24, CBX3, HXB13, HEY1, PHC2, ZNF81, FIGLA, SAM11, KMT2B, HEY2, JDP2, HXC13, ASCL4, HHEX, HERC2, GSX2, BIN1, ETV7, ASCL3, PHC1, OTP, I2BP2, VGLL2, HXA11, PDLI4, ASCL2, CDX4, ZN860, LMBL4, PDIP3, NKX25, CEBPB, ISL1, CDX2, PROP1, SIN3B, SMBT1, HXC11, HXC10, PRS6A, VSX1, NKX23, MTG16, HMX3, HMX1, KIF22, CSTF2, CEBPE, DLX2, ZMYM3, PPARG, PRIC1, UNC4, BARX2, ALX3, TCF15, TERA, VSX2, HXD12, CDX1, TCF23, ALX1, HXA10, RX, CXXC5, SCML1, NFIL3, DLX6, MTG8, CBX8, CEBPD, SEC13, FIP1, ALX4, LHX3, PRIC2, MAGI3, NELL1, PRRX1, MTG8R, RAX2, DLX3, DLX1, NKX26, NAB1, SAMD7, PITX3, WDR5, MEOX2, NAB2, DHX8, FOXA2, CBX6, EMX2, CPSF6, HXC12, KDM4B, LMBL3, PHX2A, EMX1, NC2B, DLX4, SRY, ZN777, NELL1, ZN398, GATA3, BSH, SF3B4, TEAD1, TEAD3, RGAP1, PHF1, FOXA1, GATA2, FOXO3, ZN212, IRX4, ZBED6, LHX4, SIN3A, RBBP7, NKX61, TRI68, R51A1, MB3L1, DLX5, NOTC1, TERF2, ZN282, RGS12, ZN840, SPI2B, PAX7, NKX62, ASXL2, FOXO1, GATA3, GATA1, ZMYM5, ZN783, SPI2B, LRP1, MIXL1, SGT1, LMCD1, CEBPA, GATA2, SOX14, WTIP, PRP19, CBX6, NKX11, RBBP4, DMRT2, SMCA2 and fragments thereof. In some embodiments, at least one of the repressor domains is selected from the group consisting of: SEQ ID NO: 67-595. In some embodiments, at least one of the repressor domains is selected from the group consisting of: ZIM3, ZNF264, ZN577, ZN793, ZFP28, ZN627, RYBP, TOX, TOX3, TOX4, I2BP1, SCMH1, SCML2, CDYL2, CBX8, CBX5, and CBX1, and fragments thereof.

In some embodiments, one of the repressor domains is a KRAB domain. In some embodiments, the KRAB domain is a KOX1 KRAB domain.

In some embodiments, the DNA binding domain comprises a zinc finger motif. In some embodiments, the DNA binding domain comprises a zinc finger array. In some embodiments, the DNA binding domain comprises a nucleic acid guided DNA binding domain bound to a guide polynucleotide. In some embodiments, the DNA binding domain comprises CRISPR-Cas protein bound to the guide polynucleotide. In some embodiments, wherein the guide polynucleotide hybridizes with a target sequence. In some embodiments, the CRISPR-Cas protein comprises a nuclease inactive Cas9 (dCas9). In some embodiments, the dCas9 is a dSpCas9. In some embodiments, the CRISPR-Cas protein comprises a nuclease inactive Cas12a (dCas12a). In some embodiments, the dSpCas9 is defined as SEQ ID NO: 3. In some embodiments, the CRISPR-Cas protein comprises a nuclease inactive CasX (dCasX).

In some embodiments, the fusion protein comprises from N-terminus to C-terminus DNMT3A-DNMT3L-dSpCas9-KOX1KRAB—the second repressor domain. In some embodiments, a linker connects the domains of the fusion protein. In some embodiments, the linker is an XTEN linker. In some embodiments, the XTEN linker is selected from the group consisting of: XTEN-16, XTEN-18, and XTEN-80. In some embodiments, the fusion protein comprises from N-terminus to C-terminus DNMT3A-DNMT3L-XTEN80-dSpCas9-XTEN16-KOX1KRAB-XTEN18—the second repressor domain.

Also described herein is a composition for use in the treatment of a subject, the composition comprising a fusion protein, wherein the fusion protein comprises (a) a first DNMT domain; (b) a DNA binding domain; (c) a first repressor domain; and (d) a second repressor domain.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (“FIGURE.” or “FIGURES.” herein), of which:

FIG. 1 is a schematic illustration of an example DNA methylation series plasmid containing a DNMT domain, XTEN80 linker, and a dSpCas9.

FIG. 2 shows a comparison of the ability of alternate mammalian DNMT effectors and effector fusions to reduce VIM expression in HEK293 cells.

FIG. 3A-B shows a comparison of the ability of alternate DNMT effectors and effector fusions to reduce VIM expression in HEK293 cells. FIG. 3A compares the ability of the mammalian effector fusions human DNMT3A catalytic domain-mouse DNMT3L catalytic domain and human DNMT3A catalytic domain-human DNMT3L catalytic domain to reduce VIM expression in HEK293 cells to that of plant effectors and effector fusions. FIG. 3B FIG. 3A compares the ability of the mammalian effector fusions human DNMT3A catalytic domain-mouse DNMT3L catalytic domain and human DNMT3A catalytic domain-human DNMT3L catalytic domain to reduce VIM expression in HEK293 cells to that of bacterial, fungal, and Drosophila effectors and effector fusions.

FIG. 4 is a schematic illustration of an example repressor series plasmid containing a dSpCas9, an XTEN80 linker, and a repressor domain.

FIG. 5 shows a comparison of the ability of alternate KRAB and non-KRAB repressors to effectively silence VIM expression in HEK293 cells.

FIG. 6A-B are schematic illustrations of the use of alternate KRAB and non-KRAB repressor domains. FIG. 6A is a schematic illustration of an OFF series plasmid containing a DNMT3A/3L domain; an XTEN80 linker, a dSpCas9, an XTEN16 linker, and an alternate KRAB or non-KRAB repressor domain. FIG. 6B is a schematic illustration of an OFF series plasmid containing a DNMT3A/3L domain; an XTEN80 linker, a dSpCas9, an XTEN16 linker, a KOX1 KRAB domain, an XTEN18 linker, and an alternate KRAB or non-KRAB repressor domain.

FIG. 7A-7D show the ability of OFF series plasmids with various non-KRAB repressor domains to silence CD151 expression in KEH293 cells. FIG. 7A shows the results of plasmids that do not also contain a KOX1-KRAB domain; FIG. 7B shows the results of plasmids that also contain a KOX1-KRAB domain. FIG. 7C shows additional results of plasmids that do not also contain a KOX1-KRAB domain; FIG. 7D shows additional results of plasmids that also contain a KOX1-KRAB domain.

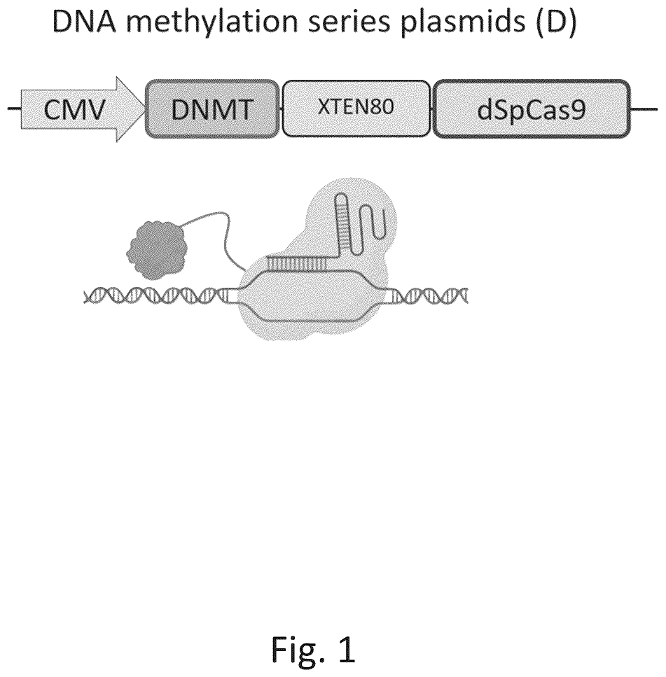

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F., and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J., and Kahn, A. (1996) DNA Isolation and Sequencing: Essential Techniques, John Wiley & Sons; Polak, J. M., and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M., and Dahlberg, J. E. (1992) Methods in Enzymology: DNA Structures Part A: Synthesis and Physical Analysis of DNA, Academic Press. Each of these general texts is herein incorporated by reference in its entirety.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms, “clinic,” “clinical setting,” “laboratory” or “laboratory setting” refer to a hospital, a clinic, a pharmacy, a research institution, a pathology laboratory, a or other commercial business setting where trained personnel are employed to process and/or analyze biological and/or environmental samples. These terms are contrasted with point of care, a remote location, a home, a school, and otherwise non-business, non-institutional setting.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing is relative or absolute. “Detecting the presence of” can include determining the amount of something present in addition to determining whether it is present or absent depending on the context.

The terms “subject,” “patient”, or “individual” are often used interchangeably herein. A “subject” may be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease. A subject may or may not have been exposed to a pathogen of interest as described herein, and may by symptomatic or symptomatic of a disease or condition associated with infection of or exposure to a pathogen as described herein. In some embodiments, a subject is suspected to have been exposed to a pathogen, e.g. a virus. In some embodiments, a subject has been exposed to an antigen or a protein representative or cross-reacts with antigens of a particular pathogen, e.g. a virus. In some embodiments, a subject has one or more symptoms that are indicative of a disease or condition associated with infection of or exposure to a pathogen as described herein. In some embodiments, the subject is currently infected by a pathogen, e.g. a virus described herein. In some embodiments, the subject is previously infected by a pathogen described herein. In some embodiments, a subject is a carrier of a virus described herein. In some embodiments, a subject is a carrier of fragments or remnants of a virus described herein. In some instances, a subject is carrier of adaptive immunity stemmed from previously or currently being infected by a virus described herein. In some embodiments, a subject is a carrier of adaptive immunity stemmed from previous or current exposure to a different virus or pathogen other than a virus or pathogen of interest.

The term “subject” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs).

The term “nucleic acid” includes any oligonucleotide or polynucleotide, with fragments containing up to 60 nucleotides generally termed oligonucleotides, and longer fragments termed polynucleotides. A deoxyribooligonucleotide consists of a 5-carbon sugar called deoxyribose joined covalently to phosphate at the 5′ and 3′ carbons of this sugar to form an alternating, unbranched polymer. DNA may be in the form of, e.g., antisense molecules, plasmid DNA, pre-condensed DNA, a PCR product, vectors, expression cassettes, chimeric sequences, chromosomal DNA, or derivatives and combinations of these groups. A ribooligonucleotide consists of a similar repeating structure where the 5-carbon sugar is ribose. Accordingly, the terms “polynucleotide” and “oligonucleotide” can refer to a polymer or oligomer of nucleotide or nucleoside monomers consisting of naturally-occurring bases, sugars and intersugar (backbone) linkages. The terms “polynucleotide” and “oligonucleotide” can also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases.

The “nucleic acid” described herein may include one or more nucleotide variants, including nonstandard nucleotide(s), non-natural nucleotide(s), nucleotide analog(s), and/or modified nucleotides. Examples of modified nucleotides include, but are not limited to diaminopurine, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates).

The nucleic acid described herein may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or phosphate backbone. Backbone modifications can include, but are not limited to, a phosphorothioate, a phosphorodithioate, a phosphoroselenoate, a phosphorodiselenoate, a phosphoroanilothioate, a phosphoraniladate, a phosphoramidate, and a phosphorodiamidate linkage. A phosphorothioate linkage substitutes a sulfur atom for a non-bridging oxygen in the phosphate backbone and delay nuclease degradation of oligonucleotides. A phosphorodiamidate linkage (N3′→P5′) allows prevents nuclease recognition and degradation. Backbone modifications can also include having peptide bonds instead of phosphorous in the backbone structure (e.g., N-(2-aminoethyl)-glycine units linked by peptide bonds in a peptide nucleic acid), or linking groups including carbamate, amides, and linear and cyclic hydrocarbon groups. Oligonucleotides with modified backbones are reviewed in Micklefield, Backbone modification of nucleic acids: synthesis, structure and therapeutic applications, Curr. Med. Chem., 8 (10): 1157-79, 2001 and Lyer et al., Modified oligonucleotides-synthesis, properties and applications, Curr. Opin. Mol. Ther., 1 (3): 344-358, 1999. Nucleic acid molecules described herein may contain a sugar moiety that comprises ribose or deoxyribose, as present in naturally occurring nucleotides, or a modified sugar moiety or sugar analog. The examples of modified sugar moieties include, but are not limited to, 2′-O-methyl, 2′-O-methoxyethyl, 2′-O-aminoethyl, 2′-Flouro, N3′→P5′ phosphoramidate, 2′dimethylaminooxyethoxy, 2′ 2′dimethylaminoethoxyethoxy, 2′-guanidinidium, 2′-O-guanidinium ethyl, carbamate modified sugars, and bicyclic modified sugars. 2′-O-methyl or 2′-O-methoxyethyl modifications promote the A-form or RNA-like conformation in oligonucleotides, increase binding affinity to RNA, and have enhanced nuclease resistance. Modified sugar moieties can also include having an extra bridge bond (e.g., a methylene bridge joining the 2′-O and 4′-C atoms of the ribose in a locked nucleic acid) or sugar analog such as a morpholine ring (e.g., as in a phosphorodiamidate morpholino).

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994).

The present disclosure encompasses isolated or substantially purified nucleic acid molecules and compositions containing those molecules. As used herein, an “isolated” or “purified” DNA molecule or RNA molecule is a DNA molecule or RNA molecule that exists apart from its native environment. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in some embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived.

As used herein, the terms “protein,” “polypeptide,” and “peptide” are used interchangeably and refer to a polymer of amino acid residues linked via peptide bonds and which may be composed of two or more polypeptide chains. The terms “polypeptide,” “protein,” and “peptide” refer to a polymer of at least two amino acid monomers joined together through amide bonds. An amino acid may be the L-optical isomer or the D-optical isomer. More specifically, the terms “polypeptide,” “protein,” and “peptide” refer to a molecule composed of two or more amino acids in a specific order; for example, the order as determined by the base sequence of nucleotides in the gene or RNA coding for the protein. Proteins are essential for the structure, function, and regulation of the body's cells, tissues, and organs, and each protein has unique functions. Examples are hormones, enzymes, antibodies, and any fragments thereof. In some cases, a protein can be a portion of the protein, for example, a domain, a subdomain, or a motif of the protein. In some cases, a protein can be a variant (or mutation) of the protein, wherein one or more amino acid residues are inserted into, deleted from, and/or substituted into the naturally occurring (or at least a known) amino acid sequence of the protein. A polypeptide can be a single linear polymer chain of amino acids bonded together by peptide bonds between the carboxyl and amino groups of adjacent amino acid residues. Polypeptides can be modified, for example, by the addition of carbohydrate, phosphorylation, etc. Proteins can comprise one or more polypeptides.

A protein or a variant thereof can be naturally occurring or recombinant. Methods for detection and/or measurement of polypeptides in biological material are well known in the art and include, but are not limited to, Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

As used herein, the terms “fragment,” or equivalent terms can refer to a portion of a protein that has less than the full length of the protein and optionally maintains the function of the protein. Further, when the portion of the protein is blasted against the protein, the portion of the protein sequence can align, for example, at least with 80% identity to a part of the protein sequence.

Any systems, methods, and platforms described herein are modular and not limited to sequential steps. Accordingly, terms such as “first” and “second” do not necessarily imply priority, order of importance, or order of acts.

The term “modulate” refers to a change in the quantity, degree or extent of a function. For example, the compositions for epigenetic modification disclosed herein may modulate the activity of a promoter sequence by binding to a motif within the promoter, thereby inducing, enhancing or suppressing transcription of a gene operatively linked to the promoter sequence. Alternatively, modulation may include inhibition of transcription of a gene wherein the epigenetic editor binds to the structural gene and blocks DNA dependent RNA polymerase from reading through the gene, thus inhibiting transcription of the gene. The structural gene may be a normal cellular gene or an oncogene, for example. Alternatively, modulation may include inhibition of translation of a transcript. Thus, “modulation” of gene expression includes both gene activation and gene repression.

The term “Administering” and its grammatical equivalents as used herein can refer to providing one or more replication competent recombinant adenovirus or pharmaceutical compositions described herein to a subject or a patient. By way of example and without limitation, “administering” can be performed by intravenous (i.v.) injection, sub-cutaneous (s.c.) injection, intradermal (i.d.) injection, intraperitoneal (i.p.) injection, intramuscular (i.m.) injection, intravascular injection, infusion (inf.), oral routes (p.o.), topical (top.) administration, or rectal (p.r.) administration. One or more such routes can be employed. Parenteral administration can be, for example, by bolus injection or by gradual perfusion over time.

The terms “treat,” “treating,” or “treatment,” and grammatical equivalents as used herein, can include alleviating, abating, or ameliorating at least one symptom of a disease or a condition, preventing additional symptoms, inhibiting the disease or the condition, e.g., arresting the development of the disease or the condition, relieving the disease or the condition, causing regression of the disease or the condition, relieving a condition caused by the disease or the condition, or stopping the symptoms of the disease or the condition either prophylactically and/or therapeutically. “Treating” may refer to administration of a vector, nucleic acid (e.g. mRNA), or LNP composition to a subject after the onset, or suspected onset, of a disease or condition. “Treating” includes the concepts of “alleviating,” which refers to lessening the frequency of occurrence or recurrence, or the severity, of any symptoms or other ill effects related to a disease or condition and/or the side effects associated with the disease or condition. The term “treating” also encompasses the concept of “managing” which refers to reducing the severity of a particular disease or disorder in a patient or delaying its recurrence, e.g., lengthening the period of remission in a patient who had suffered from the disease. The term “treating” further encompasses the concept of “prevent,” “preventing,” and “prevention.” It is appreciated that, although not precluded, treating a disorder or condition does not require that the disorder, condition, or symptoms associated therewith be completely eliminated. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “prophylaxis” is used herein to refer to a measure or measures taken for the prevention or partial prevention of a disease or condition.

By “treating or preventing a condition” is meant ameliorating any of the conditions or signs or symptoms associated with the disorder before or after it has occurred. For example, as compared with an equivalent untreated control, alleviating a symptom of a disorder may involve reduction or degree of prevention at least 3%, 5%, 10%, 20%, 40%, 50%, 60%, 80%, 90%, 95%, 98%, 99%, 99.5%, 99.9%, or 100% as measured by any standard technique. In some embodiments, alleviating a symptom of a disorder may involve reduction or degree of prevention by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared with an equivalent untreated control.

The terms “pharmaceutical composition” and its grammatical equivalents as used herein can refer to a mixture or solution comprising a therapeutically effective amount of an active pharmaceutical ingredient together with one or more pharmaceutically acceptable excipients, carriers, and/or a therapeutic agent to be administered to a subject, e.g., a human in need thereof.

The term “pharmaceutically acceptable” and its grammatical equivalents as used herein can refer to an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, i.e., the material may be administered to a subject without causing undesirable biological effects or interacting in a deleterious manner with any of the components of the pharmaceutical composition in which it is contained.

A “pharmaceutically acceptable excipient, carrier, or diluent” refers to an excipient, carrier, or diluent that can be administered to a subject, together with an agent, and which does not destroy the pharmacological activity thereof and is nontoxic when administered in doses sufficient to deliver a therapeutic amount of the agent.

A “pharmaceutically acceptable salt” may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the term “therapeutically effective amount” means an amount of an agent to be delivered (e.g., nucleic acid, drug, payload, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

The term “repressor domain” or “repression domain” are terms known in the art. Such domains typically refer to a part of a transcription repression protein which provides for the transcriptional repressive effect on a target gene, for example by participating in a reaction on the DNA or chromatin (e.g., methylation), by binding to an agent from within the nucleus to result in the repression of the transcription of the target gene or by inhibiting the recruitment of a protein in the natural transcriptional machinery that transcribes the target gene. Examples of repressor domains of this invention are provided through the specification.

The term “KRAB” or “KRAB domain” is a term known in the art. KRAB is also known as Krippel associated box, a transcription repressor domain. A description of KRAB domains, including their function and use, may be found, for example, in Ecco, G., Imbeault, M., Trono, D., KRAB zinc finger proteins, Development 144, 2017 and Lambert S A, Jolma A, Campitelli L F, Das P K, Yin Y, Albu M, Chen X, Taipale J, Hughes T R, Weirauch M T, 2018, The human transcription factors, Cell 172: 650-665, 10.1016/j.cell.2018.01.029, which are incorporated by reference in their entirety. Examples of KRAB domains are also provided throughout the specification.

The term “DNMT” is a term known in the art. DNMT is also known as DNA methyltransferase. DNMT refers to an enzyme that catalyzes the transfer of a methyl group to DNA. Non-limiting examples of DNA methyltransferases include DNMT, DNMT3A, DNMT3B, DNMT3C and DNMT3L. In one preferred embodiment, a catalytic domain(s) of a DNMT is used in the invention.

The term “DNA binding domain” is a term known in the art. DNA binding domain typically refers to a part of a protein which binds to DNA in a nucleus. In one embodiment of this invention, a DNA-binding domain is a DNA binding region of a protein selected from a CRISPR Cas protein, a TAL protein, a zinc finger protein, a transcription repression protein, a transcription activation protein, or an variants thereon that bind DNA.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The term “therapeutic agent” can refer to any agent that, when administered to a subject, has a therapeutic, diagnostic, and/or prophylactic effect and/or elicits a desired biological and/or pharmacological effect. Therapeutic agents can also be referred to as “actives” or “active agents.” Such agents include, but are not limited to, cytotoxins, radioactive ions, chemotherapeutic agents, small molecule drugs, proteins, and nucleic acids.

The term “ameliorate” as used herein can refer to decrease, suppress, attenuate, diminish, arrest, or stabilize the development or progression of a disease.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein, “onset” or “occurrence” of a disease includes initial onset and/or recurrence. Conventional methods, known to those of ordinary skill in the art of medicine, can be used to administer the isolated polypeptide or pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir.

The term “parenteral” as used herein includes subcutaneous, intracutaneous, intravenous, intramuscular, intraarticular, intraarterial, intrasynovial, intrastemal, intrathecal, intralesional, and intracranial injection or infusion techniques. In addition, it can be administered to the subject via injectable depot routes of administration such as using 1-, 3-, or 6-month depot injectable or biodegradable materials and methods.

It will be understood that in addition to the specific proteins and nucleotides mentioned herein, the present invention also contemplates the use of variants, derivatives, homologues and fragments thereof. As used herein, a variant of any given sequence is a sequence in which the specific sequence of residues (whether amino acid or nucleic acid residues) has been modified in such a manner that the polypeptide or polynucleotide in question substantially retains at least one of its endogenous functions. A variant sequence can be obtained by addition, deletion, substitution, modification, replacement and/or variation of at least one residue present in the naturally-occurring protein. As used herein, a derivative of any given sequence as contemplated includes any substitution of, variation of, modification of, replacement of, deletion of and/or addition of one (or more) amino acid residues from or to the sequence providing that the resultant protein or polypeptide substantially retains at least one of its endogenous functions. Amino acid substitutions may be made, for example from 1, 2 or 3 to 10 or 20 substitutions provided that the modified sequence substantially retains the required activity or ability. Amino acid substitutions may include the use of non-naturally occurring analogues. Proteins used in the present disclosure may also have deletions, insertions or substitutions of amino acid residues which do not affection function of the protein and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

As used herein, a homologue of any herein contemplated protein or nucleic acid sequence includes sequences having a certain homology with the wild type amino acid and nucleic sequence. A homologous sequence may include a sequence, e.g. an amino acid sequence which may be at least 50%, 55%, 65%, 75%, 85% or 90% identical to the subject sequence. In particular embodiments, a homologous sequence may include an amino acid sequence at least 95% or 97% or 99% identical to the subject sequence.

Sequence identity may be measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

### Nucleic Acid Binding Domains

Epigenetic editors and epigenetic editing complexes described herein may comprise one or more nucleic acid binding protein domains, e.g. DNA binding domains, that may direct the epigenetic editor to a target gene associated with a certain condition.

As used herein, a target gene can comprise all nucleotide sequences of a gene of interest. For example, sequences or nucleotides of a target gene can include coding sequences and non-coding sequences. Sequence of a target gene can include exons or introns. Sequences of a target gene can include regulatory regions, including promoters, enhancers, terminators, 5′ or 3′ untranslated regions. In some embodiments, a sequence of a target gene comprises a remote enhancer sequence.

An epigenetic editor as described herein can comprise any polynucleotide binding domain. In some embodiments, the nucleic acid binding domain comprises one or more DNA binding proteins, for example, zinc finger proteins (ZFPs) or transcription activator like effectors (TALEs). In some embodiments, the nucleic acid binding domain comprises a polynucleotide guided DNA binding protein, for example, a nuclease inactive CRISPR-Cas protein guided by a guide RNA.

The nucleic acid binding domain of epigenetic editors described herein may be capable of recognizing and binding any gene of interest, for example, target genes associated with a disease or disorder. In some embodiments, the target gene associated with a disease or disorder contains a mutation as compared to a wild type gene. In some embodiments, the target gene associated with a disease or disorder contains a copy that harbors a mutation associated with the disease or disorder. In some embodiments, the target gene associated with a disease or disorder has one or both copies of wild type DNA sequences.

A DNA binding domain maybe modular and/or programmable. In some embodiments, the DNA binding domain comprises a zinc finger domain, a transcription activator like effector (TALE) domain, a meganuclease DNA binding domain or a polynucleotide guided nucleic acid binding domain. Examples of DNA binding domains can be found in U.S. Pat. No. 11,162,114, which is incorporated by reference in its entirety.

Transcription activator-like effectors (TALEs) can be engineered to bind practically any desired DNA sequence. Methods for programming TALEs are familiar to one skilled in the art. For example, such methods are described in Carroll et al, Genetics Society of America, 188 (4): 773-782, 2011; Miller et al., Nature Biotechnology 25 (7): 778-785, 2007; Christian et al, Genetics 186 (2): 757-61, 2008; Li et al, Nucleic Acids Res. 39 (1): 359-372, 2010; and Moscou et al, Science 326 (5959): 1501, 2009, each of which are incorporated herein by reference.

A DNA binding domain may be directed by a nucleic acid sequence, for example, a RNA sequence, to identify the target gene. In some embodiments, the DNA binding domain comprises a programmable nuclease. In some embodiments, the DNA binding domain comprises a programmable nuclease with reduced or abrogated nuclease activity. For example, a programmable nuclease may harbor one or two mutations in its catalytic domain that renders the nuclease inactive, but maintain DNA binding activity of the nuclease. In some embodiments, the DNA binding domain comprises a CRISPR-Cas protein domain. In some embodiments, the CRISPR-Cas protein domain lacks or has reduced nuclease activity.

In some embodiments, an epigenetic editor provided herein comprises a Cas protein, e.g. a Cas9 protein domain. The Cas9 domain may be any of the Cas9 domains or Cas9 proteins (e.g., nuclease inactive Cas9 or Cas9 nickase, or a Cas9 variant from any species) provided herein. In some embodiments, any of the Cas domains or Cas proteins provided herein may be fused with one or more any effector protein domain as described herein. In some embodiments, any of the Cas protein domains provided herein may be fused with two or more effector protein domains as described herein. Cas9 can refer to a polypeptide with at least about 50%, 60%, 70%, 80%, 90%, 100% sequence identity and/or sequence similarity to a wild type exemplary Cas9 polypeptide (e.g., from S. pyogenes). Cas9 can refer to the wild type or a modified form of the Cas9 protein that can comprise an amino acid change such as a deletion, insertion, substitution, variant, mutation, fusion, chimera, or any combination thereof.

Cas9 sequences and structures of variant Cas9 orthologs have been described in various species. Exemplary species that the Cas9 protein or other components can be from include, but are not limited to, Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polar omonas naphthalenivorans, Polar omonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionium, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillator ia sp., Petrotoga mobilis, Thermosipho africanus, Streptococcus pasteurianus, Neisseria cinerea, Campylobacter lari, Parvibaculum lavamentivorans, Coryne bacterium diphtheria, or Acaryochloris marina. In some embodiments, the Cas9 protein is from Streptococcus pyogenes. In some embodiments, the Cas9 protein may be from Streptococcus thermophilus. In some embodiments, the Cas9 protein is from Staphylococcus aureus.

Additional suitable Cas9 proteins, orthologs, variants, including nuclease inactive variants and sequences will be apparent to those of skill in the art based on this disclosure, and such Cas9 nucleases and sequences include Cas9 sequences from the organisms and loci disclosed in Chylinski et al., (2013) RNA Biology 10:5, 726-737; which are incorporated herein by reference.

In some embodiments, wild-type Cas9 corresponds to Cas9 from Streptococcus pyogenes (NCBI Reference Sequence: NC_002737.2 (SEQ ID NO.: 1); and Uniprot Reference Sequence: Q99ZW2 (SEQ ID NO.: 2).

An epigenetic editor may comprise a nuclease inactive Cas9 domain (dead Cas9 or dCas9). The dCas9 protein domain may comprise one, two, or more mutations as compared to a wild type Cas9 that abrogate its nuclease activity, but retains the DNA binding activity. For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9. In some embodiments, the dCas9 comprises at least one mutation in the HNH subdomain and the RuvC subdomain that reduces or abrogates nuclease activity. In some embodiments, the dCas9 only comprises a RuvC subdomain. In some embodiments, the dCas9 only comprises a HNR subdomain. It is to be understood that any mutation that inactivates the RuvC or the HNH domain may be included in a dCas9, e.g., insertion, deletion, or single or multiple amino acid substitution in the RuvC domain and/or the HNH domain.

In some embodiments, the dCas9 protein comprises a mutation at position D10 as numbered in the wild type Cas9 sequence as numbered in Uniprot Reference Sequence Q99ZW2. In some embodiments, the dCas9 protein comprises a mutation at position H840 as numbered in Uniprot Reference Sequence: Q99ZW2. In some embodiments, the dCas9 protein comprises a D10A mutation as numbered in Uniprot Reference Sequence: Q99ZW2. In some embodiments, the dCas9 protein comprises a H840A mutation as numbered in Uniprot Reference Sequence: Q99ZW2. In some embodiments, the dCas9 protein comprises a D10A and a H840A mutation as numbered in Uniprot Reference Sequence: Q99ZW2. In some embodiments, a nuclease inactive Cas9 comprises the amino acid sequence of dCas9 (D10A and H840A) (SEQ ID NO.: 3).

Additional suitable mutations that inactivate Cas9 will be apparent to those of skill in the art based on this disclosure and knowledge in the field and are within the scope of this disclosure. Such additional exemplary suitable nuclease-inactive Cas9 domains include, but are not limited to, D839A, N863A, and/or K603R. Cas9, dCas9, or Cas9 variant also encompasses Cas9, dCas9, or Cas9 variants from any organism. Also appreciated is that dCas9, Cas9 nickase, or other appropriate Cas9 variants from any organisms may be used in accordance with the present disclosure.

In some embodiments, an epigenetic editor comprises a high fidelity Cas9 domain. For example, high fidelity Cas9 domains comprising one or more mutations that decrease electrostatic interactions between the Cas9 domain and the sugar-phosphate backbone of DNA may be incorporated in an epigenetic editor to confer increased target binding specificity as compared to a corresponding wild-type Cas9 domain. Without wishing to be bound by any particular theory, high fidelity Cas9 domains that have decreased electrostatic interactions with the sugar-phosphate backbone of DNA may have less off-target effects. In some embodiments, the Cas9 domain comprises one or more mutations that decreases the association between the Cas9 domain and the sugar-phosphate backbone of DNA. In some embodiments, a Cas9 domain comprises one or more mutations that decreases the association between the Cas9 domain and the sugar-phosphate backbone of DNA by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, or more. In some embodiments, a high fidelity Cas9 domain comprises one or more of N497X, R661X, Q695X, and/or Q926X mutation as numbered in the wild type Cas9 amino acid sequence Uniprot Reference Sequence: Q99ZW2 or a corresponding amino acid in another Cas9, wherein X is any amino acid. In some embodiments, a high fidelity Cas9 domain comprises one or more of N497A, R661A, Q695A, and/or Q926A mutation of the amino acid sequence provided in the wild type Cas9 sequence, or a corresponding mutation as numbered in the wild type Cas9 amino acid sequence Uniprot Reference Sequence: Q99ZW2 or a corresponding amino acid in another Cas9. It should be appreciated that any of the epigenetic editors provided herein, for example, any of the epigenetic activators or repressors provided herein, may be converted into high fidelity epigenetic editors by modifying the Cas9 domain as described. In preferred embodiments, the high fidelity Cas9 domain is a nuclease inactive Cas9 domain.

In some embodiments, a DNA binding domain in an epigenetic editor is a CRISPR protein that recognizes a protospacer adjacent motif (PAM) sequence in a target gene. A CRISPR protein may recognize a naturally occurring or canonical PAM sequence or may have altered PAM specificities. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et ah, “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the Cas9 domain is a Cas9 domain from S. pyogenes (SpCas9). In some embodiments, a SpCas9 recognizes a canonical NGG PAM sequence where the “N” in “NGG” is adenine (A), thymine (T), guanine (G), or cytosine (C), and the G is guanine. In some embodiments, an epigenetic editor or fusion protein provided herein contains a SpCas9 domain that is capable of binding a nucleotide sequence that does not contain a canonical (e.g., NGG) PAM sequence. In some embodiments, the SpCas9 domain, the nuclease inactive SpCas9 domain, or the SpCas9 nickase domain can bind to a nucleic acid sequence having a NGG, a NGA, or a NGCG PAM sequence. In some embodiments, the SpCas9 domain comprises one or more of a D1135X, a R1335X, and a T1337X mutation as numbered in the wild type SpCas9 amino acid sequence or a corresponding mutation in another SpCas9 protein, wherein X is any amino acid. In some embodiments, the SpCas9 domain comprises one or more of a D1135E, R1335Q, and T1337R mutation as numbered in the wild type SpCas9 amino acid sequence or a corresponding mutation in another SpCas9 protein. In some embodiments, the SpCas9 domain comprises one or more of a D1 134V, a R1334Q, and a T1336R mutation as numbered in the wild type Cas9 amino acid sequence, or a corresponding mutation thereof. In some embodiments, the SpCas9 domain comprises a D1135V, a R1335Q, and a T1337R mutation as numbered in the wild type SpCas9 amino acid sequence or a corresponding mutation in another SpCas9 protein. In some embodiments, the SpCas9 domain comprises one or more of a D1135X, a G1218X, a R1335X, and a T1337X mutation as numbered in the wild type SpCas9 amino acid sequence or a corresponding mutation in another SpCas9 protein, wherein X is any amino acid. In some embodiments, the SpCas9 domain comprises one or more of a D1135V, a G1218R, a R1335Q, and a T1337R mutation as numbered in the wild type SpCas9 amino acid sequence or a corresponding mutation in another SpCas9 protein. In some embodiments, the SpCas9 domain comprises a D1135V, a G1218R, a R1335Q, and a T1337R mutation as numbered in the wild type SpCas9 amino acid sequence or a corresponding mutation in another SpCas9 protein.

In some embodiments, the Cas9 domain is a modified SpCas9 domain having specificity for a 5′-NGCG-3′ PAM sequence, where N is any one of nucleotides A, G, C, or T. In some embodiments, the modified SpCas9 domain having specificity for a 5′-NGCG-3′ PAM sequence comprises a D1135V, a G1218R, a R1335E, and a T1337R mutation as numbered in the wild type SpCas9 amino acid sequence or a corresponding mutation in another SpCas9 protein (the “VRER” SpCas9). In some embodiments, the VRER SpCas9 further comprises one or more mutations that reduces or abolishes its nuclease activity. For example, the SpCas9 may further comprise a D10A and a H840A mutation and is a nuclease inactive SpCas9. Amino acid sequence of an exemplary nuclease inactive VRER SpCas9 is provided in SEQ ID NO.: 4.

In some embodiments, the Cas9 domain is a modified SpCas9 domain having specificity for a 5′-NGAG-3′ PAM sequence, where N is any one of nucleotides A, G, C, or T. In some embodiments, the modified SpCas9 domain having specificity for a 5′-NGAG-3′ PAM sequence comprises a D1135E, a R1335Q, and a T1337R mutation as numbered in the wild type SpCas9 amino acid sequence or a corresponding mutation in another SpCas9 protein (the “EQR” SpCas9). In some embodiments, the EQR SpCas9 further comprises one or more mutations that reduces or abolishes its nuclease activity. For example, the SpCas9 may further comprise a D10A and a H840A mutation and is a nuclease inactive SpCas9.

Amino acid sequence of an exemplary nuclease inactive EQR SpCas9 is provided in SEQ ID NO.: 5.

In some embodiments, the Cas9 domain is a modified SpCas9 domain having specificity for a 5′-NGAN-3′ or a 5-NGNG-3′ PAM sequence, where N is any one of nucleotides A, G, C, or T. In some embodiments, the modified SpCas9 domain having specificity for a 5′-NGAN-3′ or a 5-NGNG-3′ PAM sequence comprises a D1135V, a R1335Q, and a T1337R mutation as numbered in the wild type SpCas9 amino acid sequence or a corresponding mutation in another SpCas9 protein (the “VQR” SpCas9). In some embodiments, the VQR SpCas9 further comprises one or more mutations that reduces or abolishes its nuclease activity. For example, the SpCas9 may further comprise a D10A and a H840A mutation and is a nuclease inactive SpCas9.

Amino acid sequence of an exemplary nuclease inactive VQR SpCas9 is provided in SEQ ID NO.: 6.

In some embodiments, the Cas9 domain is a modified SpCas9 domain having specificity for a 5′-NGN-3′ PAM sequence, where N is any one of nucleotides A, G, C, or T. In some embodiments, the modified SpCas9 domain having specificity for a 5′-NGN-3′ PAM sequence comprises a D1135L, a S1136W, a G1218K, a E1219Q, a R1335Q, a T1337R, a D1135V, a R1335Q, and a T1337R mutation as numbered in the wild type SpCas9 amino acid sequence or a corresponding mutation in another SpCas9 protein (the “SpGCas9”). In some embodiments, the SpG Cas9 further comprises one or more mutations that reduces or abolishes its nuclease activity. For example, the SpGCas9 may further comprise a D10A and a H840A mutation and is a nuclease inactive SpGCas9.

Amino acid sequence of an exemplary nuclease inactive SpG Cas9 is provided in SEQ ID NO.: 7.

In some embodiments, the Cas9 domain is a modified SpCas9 domain having specificity for a 5′-NRN-3′ or a 5′-NYN-3′ PAM sequence, where N is any one of nucleotides A, G, C, or T, where R is nucleotide A or G, and where Y is nucleotide C or T. In some embodiments, the modified SpCas9 domain having specificity for a 5′-NRN-3′ or a 5′-NYN-3′ PAM sequence comprises a A61R, a L1111R, a D1135L, a S1136W, a G1218K, a E1219Q, a N1317R, a A1322R, a R1333P, a R1335Q, and a T1337R mutation as numbered in the wild type SpCas9 amino acid sequence or a corresponding mutation in another SpCas9 protein (the “SpRYCas9”). In some embodiments, the SpRY Cas9 further comprises one or more mutations that reduces or abolishes its nuclease activity. For example, the SpCas9 may further comprise a D10A and a H840A mutation and is a nuclease inactive SpRYCas9.

Amino acid sequence of an exemplary nuclease inactive SpRY Cas9 is provided in SEQ ID NO.: 8.

In some embodiments, the Cas9 domain is a Cas9 domain from Staphylococcus aureus (SaCas9). In some embodiments, the SaCas9 domain is a nuclease inactive SaCas9 (dSacas9). In some embodiments, the SaCas9 comprises a N579A mutation as numbered in the wild type SaCas9 sequence or a corresponding mutation in another SaCas9 protein. In some embodiments, the SaCas9 comprises a D10A mutation as numbered in the wild type SaCas9 sequence or a corresponding mutation in another SaCas9 protein. In some embodiments, the dSaCas9 comprises a D10A mutation and a N579A mutation as numbered in the wild type SaCas9 sequence or a corresponding mutation in another SaCas9 protein.

An exemplary wild type SaCas9 protein is provided in SEQ ID NO.: 9.

In some embodiments, the SaCas9 domain, the nuclease inactive SaCas9 domain, or the SaCas9 nickase domain can bind to a nucleic acid sequence having a non-canonical PAM. In some embodiments, the SaCas9 domain, the SaCas9d domain, or the SaCas9n domain can bind to a nucleic acid sequence having a NNGRRT PAM sequence, where N=A, T, C, or G, and R=A or G. In some embodiments, the SaCas9 domain comprises one or more of a E781K, a N967K, and a R1014H mutation as numbered in the wild type SaCas9 sequence or a corresponding mutation in another SaCas9 protein (the “KKH” SaCas9). In some embodiments, the SaCas9 domain comprises a E781K, a N967K, or a R1014H mutation as numbered in the wild type SaCas9 sequence or a corresponding mutation in another SaCas9 protein. In some embodiments, the SaCas9 domain, the SaCas9d domain, or the SaCas9n domain can bind to a nucleic acid sequence having a non-canonical PAM. In some embodiments, the SaCas9 domain or the nuclease inactive SaCas9d domain can bind to a nucleic acid sequence having a NNGRRT PAM sequence. In some embodiments, the SaCas9 domain comprises one or more of a E781K, a N967K, and a R1014H mutation, or one or more corresponding mutation in any of the amino acid sequences provided herein. In some embodiments, the SaCas9 domain comprises a E781K, a N967K, or a R1014H mutation, or corresponding mutations in any of the amino acid sequences provided herein. In some embodiments, the KKH SaCas9 further comprises one or more mutations that reduces or abolishes its nuclease activity. For example, the KKHSaCas9 may further comprise a D10A and a N579A mutation and is a nuclease inactive KKH SaCas9. Amino acid sequence of an exemplary nuclease inactive KKH dSaCas9 is provided in SEQ ID NO.: 10

In some embodiments, the Cas9 domain is a Cas9 domain from Neisseria meningitidis (NmeCas9). In some embodiments, the NmeCas9 domain is a nuclease inactive NmeCas9 (dNmeCas9). An NmeCas9 may have specificity for a 5′-NNNGATT-3′ PAM, where N is any one of nucleotides A, G, C, or T. In some embodiments, the NmeCas9 comprises a D16A mutation, or a corresponding mutation in any of the amino acid sequences as numbered in the wild type NmeCas9 sequence. In some embodiments, the NmeCas9 comprises a H588A mutation as numbered in the wild type NmeCas9 sequence or a corresponding mutation in another NmeCas9 protein. In some embodiments, a dNmeCas9 comprises a D16A and a H588A mutation.

Amino acid sequence of an exemplary dNmeCas9 protein is provided in SEQ ID NO.: 11.

In some embodiments, the Cas9 domain is a Cas9 domain from Campylobacter jejuni (CjCas9). In some embodiments, the CjCas9 domain is a nuclease inactive CjCas9 (dCjCas9). A Cj Cas9 may have specificity for a 5′-NNNVRYM-3′ PAM, where N is any one of nucleotides A, G, C, or T, V is nucleotide A, C, or G, R is nucleotide A or G, Y is nucleotide C or T, and M is nucleotide A or C. In some embodiments, the CjCas9 comprises a D8A mutation, or a corresponding mutation in any of the amino acid sequences as numbered in the wild type CjCas9 sequence. In some embodiments, the CjCas9 comprises a H559A mutation as numbered in the wild type CjCas9 sequence or a corresponding mutation in another CjCas9 protein. In some embodiments, a dCjCas9 comprises a D16A and a H588A mutation.

Amino acid sequence of an exemplary dCjCas9 protein is provided in SEQ ID NO.: 12.

In some embodiments, the Cas9 domain is a Cas9 domain from Streptococcus thermophilus (StCas9). In some embodiments, the StCas9 is encoded by St CRISPRI loci of the Streptococcus thermophilus (St1Cas9). In some embodiments, the St1Cas9 domain is a nuclease inactive St1Cas9 (dSt1Cas9). An St1Cas9 may have specificity for a 5′-NNAGAAW-3′ PAM, where N is any one of nucleotides A, G, C, or T, and W is nucleotide A or T. In some embodiments, the St1Cas9 comprises a D10A mutation, or a corresponding mutation in any of the amino acid sequences as numbered in the wild type St1Cas9 sequence. In some embodiments, the St1Cas9 comprises a H600A mutation as numbered in the wild type St1Cas9 sequence or a corresponding mutation in another St1Cas9 protein. In some embodiments, a St1Cas9d comprises a D10A and a H600A mutation.

In some embodiments, the StCas9 is encoded by St CRISPR3 loci of the Streptococcus thermophilus (St3Cas9). In some embodiments, the St3Cas9 domain is a nuclease inactive St3Cas9 (dSt3Cas9). An St3Cas9 may have specificity for a 5′-NGGNG-3′ PAM, where N is any one of nucleotides A, G, C, or T. In some embodiments, the St3Cas9 comprises a D10A mutation, or a corresponding mutation in any of the amino acid sequences as numbered in the wild type St3Cas9 sequence. In some embodiments, the St3Cas9 comprises a N870A mutation as numbered in the wild type St3Cas9 sequence or a corresponding mutation in another St3Cas9 protein. In some embodiments, a dSt3Cas9 comprises a D10A and a N870A mutation.

Amino acid sequence of an exemplary dStlCas9 protein is provided in SEQ ID NO.: 13.

Amino acid sequence of an exemplary dSt3Cas9 protein is provided in SEQ ID NO.: 14.

In some embodiments, the Cas9 domain of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 sequences provided herein.

In some embodiments, an epigenetic editor provided herein comprises a Cpf1 (or Cas12a) protein domain. For example, an epigenetic editor can comprise a nuclease inactive Cpf1 protein or a variant thereof. The Cpf1 protein has a RuvC-like endonuclease domain that is similar to the RuvC domain of Cas9 but does not have a HNH endonuclease domain, and the N-terminal of Cpf1 does not have the alpha-helical recognition lobe of Cas9. In some embodiments, the Cpf1 comprises one or more mutations corresponding to D917A, E1006A, or D1255A as numbered in the Francisella novicida Cpf1 protein (FnCpf1). A FnCpf1 may have specificity for a 5′-TTN-3′ PAM sequence, where N is any one of nucleotides A, T, G, or C. In some embodiments, the Cpf1 protein has reduced nuclease activity. In some embodiments, the nuclease activity of the Cpf1 protein is abolished (dCpf1). In some embodiments, the dCpf1 protein comprises mutations corresponding to D917A, E1006A, D1255A, D917A/E1006A, D917A/D1255A, E1006A/D1255A, or D917A/E1006A/D1255A or a corresponding mutation in any of the Cpf1 amino acid sequences as numbered in the wild type FnCpf1 sequence provided herein. In some embodiments, the dCpf1 comprises a D917A mutation, or a corresponding mutation in any of the Cpf1 amino acid sequences as numbered in the wild type FnCpf1 sequence.

In some embodiments, the Cpf1 protein comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to the FnCpf1 sequence provided herein. It should be appreciated that Cpf1 from other bacterial species may also be used in accordance with the present disclosure.

An exemplary wild type Francisella novicida Cpf1 amino acid sequence is provided in SEQ ID NO.: 15.

Amino acid sequence of an exemplary nuclease inactive FnCpf1 protein is provided in SEQ ID NO.: 16.

In some embodiments, the Cpf1 is a Cpf1 protein from Lachnospiraceae bacterium (LbCpf1). A LbCpf1 may have specificity for a 5′-TTTV-3′ PAM sequence, where V is any one of nucleotides A, G, or C. In some embodiments, the LbCpf1 protein has reduced nuclease activity. In some embodiments, the nuclease activity of the LbCpf1 protein is abolished (dLbCpf1). In some embodiments, the dLbCpf1 protein comprises mutations corresponding to D832A or a corresponding mutation in any of the Cpf1 amino acid sequences as numbered in the wild type LbCpf1 sequence provided herein.

Amino acid sequence of an exemplary nuclease inactive dLbCpf1 protein is provided in SEQ ID NO.: 17.

In some embodiments, the Cpf1 is a Cpf1 protein from Acidaminococcus sp. (AsCpf1). A AsCpf1 may have specificity for a 5′-TTTV-3′ PAM sequence, where V is any one of nucleotides A, G, or C. In some embodiments, the AsCpf1 protein has reduced nuclease activity. In some embodiments, the nuclease activity of the AsCpf1 protein is abolished (dAsCpf1. In some embodiments, the dLbCpf1 protein comprises mutations corresponding to D908A or a corresponding mutation in any of the Cpf1 amino acid sequences as numbered in the wild type AsCpf1 sequence provided herein. In some embodiments, the dAsCpf1 or AsCpf1 further comprises mutations that improve targeting and editing efficiency. For example, an AsCpf1 may comprise mutations E174R, S542R, and K548R (“enAsCpf1”) or corresponding mutations in any of the Cpf1 amino acid sequences as numbered in the wild type AsCpf1 sequence provided herein.

Amino acid sequence of an exemplary nuclease inactive AsCpf1 protein is provided in SEQ ID NO.: 18.

Amino acid sequence of an exemplary nuclease inactive enAsCpf1 protein is provided in SEQ ID NO.: 19.

In some embodiments, the dAsCpf1 or AsCpf1 protein further comprises mutations that improve fidelity of target recognition of the protein. For example, an AsCpf1 may comprise mutations E174R, N282A, S542R, and K548R (“HFAsCpf1”) or corresponding mutations in any of the Cpf1 amino acid sequences as numbered in the wild type AsCpf1 sequence provided herein.

Amino acid sequence of an exemplary nuclease inactive HFAsCpf1 protein is provided in SEQ ID NO.: 20.

In some embodiments, the dAsCpf1 or AsCpf1 protein further comprises mutations that result in altered PAM specificity of the protein. In some embodiments, an AsCpf1 comprising mutations S542R, K548V, and N552R (“RVRAsCpf1”) or corresponding mutations in any of the Cpf1 amino acid sequences as numbered in the wild type AsCpf1 sequence provided herein may have specificity for a 5′-TATV-3′ PAM, where V is any one of nucleotides A, C, or G. In some embodiments, an AsCpf1 comprising mutations S542R and K607R (“RRAsCpf1”) or corresponding mutations in any of the Cpf1 amino acid sequences as numbered in the wild type AsCpf1 sequence provided herein may have specificity for a 5′-TYCV-3′ PAM, where Y is any one of nucleotides C or T and V is any one of nucleotide A, C, or G.

Amino acid sequence of an exemplary nuclease inactive RVRAsCpf1 protein is provided in SEQ ID NO.: 21.

Amino acid sequence of an exemplary nuclease inactive RRAsCpf1 protein is provided in SEQ ID NO.: 22.

In some embodiments, an epigenetic editor provided herein comprises a Cas protein domain other than Cas9. In some embodiments, the Cas9 protein comprises an inactivated nuclease domain. In some embodiments, an epigenetic editor comprises a Cas12a, a Cas12b, a Cas12c, a Cas12d, a Cas12e, a Cas12h, or a Cas12i domain. In some embodiments, the Cas9 protein is a RNA nuclease or an inactivated RNA nuclease. In some embodiments, an epigenetic editor comprises a Cas12g, a Cas13a, a Cas13b, a Cas13c, or a Cas13d domain. In some embodiments, an epigenetic editor comprises an Argonaut protein domain.

A CRISPR/Cas system or a Cas protein in an epigenetic editor system provided herein may comprise Class 1 or Class 2 Cas proteins. The Class 1 or Class 2 proteins used in an epigenetic editor may be inactivated in its nuclease activity. In some embodiments, an epigenetic editor comprises a Cas protein derived from a Type II, Type IIA, Type IIB, Type IIC, Type V, or Type VI Cas nuclease. In some embodiments, an epigenetic editor comprises a Cas protein derived from a Class 2 Cas nucleases derived from Cas1, Cas1B, Cas2, Cas3, Cas4, Cas5, Cas6, Cas7, Cas8, Cas10, Cas14a, Cas14b, Cas14c, CasX, CasY, CasPhi, C2c4, C2c8, C2c9, C2c10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, or homologues or modified versions thereof. In some embodiments, a Cas protein in an epigenetic editor is a nuclease inactivated Cas protein.

In some embodiments, the epigenetic editor comprises a CasX (Cas12e) protein. A CasX protein may have specificity for a 5′-TTCN-3′ PAM sequence, where N is any one of nucleotides A, G, T, or C. In some embodiments, the CasX protein has reduced or abolished nuclease activity (dCasX), In some embodiments, the dCasX protein comprises one or more of E672X, E769X, D935X amino acid substitutions as compared to the CasX reference sequence provided below, where X is any amino acid other than the wild type amino acid. In some embodiments, the dCasX protein comprises one or more of E672A, E769A, D935A amino acid substitutions as compared to the CasX reference sequence provided below. In some embodiments, the CasX protein is a truncated CasX protein as compared to the wild type. In some embodiments, the CasX protein lacks a target strand loading domain (TSLD). CasX protein and sequences as described in U.S. Pat. No. 10,570,415 and PCT application publication No.s WO2020023529, WO2020041456 are incorporated herein in the entirety.

An exemplary CasX amino acid sequence is provided in SEQ ID NO.: 23.

An exemplary dCasX amino acid sequence is provided in SEQ ID NO.: 24.

In some embodiments, the epigenetic editor comprises a CasY (Cas12d) protein. A CasY protein may have specificity for a 5′-TA-3′ PAM sequence. In some embodiments, the CasY protein has reduced or abolished nuclease activity (dCasY). In some embodiments, the dCasY protein comprises one or more of D828X, E914X, D1074X amino acid substitutions as compared to the CasY reference sequence provided below, where X is any amino acid other than the wild type amino acid. In some embodiments, the dCasY protein comprises one or more of D828A, E914A, D1074A amino acid substitutions as compared to the CasY reference sequence provided below. CasY protein and sequences as described in US Patent Application Publication No.s US20200255858 and US20190300908 are incorporated herein in the entirety.

An exemplary CasY amino acid sequence is provided in SEQ ID NO.: 25.

In some embodiments, the epigenetic editor comprises a Casφ (CasPhi) protein. A Casφ protein may have specificity for a 5′-TTN-3′ PAM sequence, wherein N is any one of nucleotides A, T, G, or C. In some embodiments, the Casφ protein has reduced or abolished nuclease activity (dCasφ). In some embodiments, a dCasφ protein comprises a D394A mutation or a corresponding mutation in any of the Casφ amino acid sequences as numbered in the wild type Casφ sequence provided herein.

Cas φ protein and sequences as described in Pausch et al., CRISPR-Cas φ from huge phages is a hypercompact genome editor, Science 369, 333-337 (2020), which is incorporated herein in the entirety.

An exemplary wild type Casφ (CasPhi) amino acid sequence is provided in SEQ ID NO.: 26.

An exemplary dCasφ (dCasPhi) amino acid sequence is provided in SEQ ID NO.: 27.

In some embodiments, the epigenetic editor comprises a Cas12f1 (Cas14a) protein as in SEQ ID NO.: 28. In some embodiments, the epigenetic editor comprises a Cas12f2 (Cas14b) protein as in SEQ ID NO.: 29. In some embodiments, the epigenetic editor comprises a Cas12f3 (Cas14c) protein as in SEQ ID NO.: 30. In some embodiments, the epigenetic editor comprises a C2c8 protein as in SEQ ID NO.: 31.

In some embodiments, the Cas protein is a circular permutant Cas protein. For example, an epigenetic editor may comprise a circular permutant Cas9 as described in Oakes et al., Cell 176, 254-267 (2019), incorporated herein in its entirety. As used herein, the term “circular permutant” refers to a variant polypeptide (e.g., of a subject Cas protein) in which one section of the primary amino acid sequence has been moved to a different position within the primary amino acid sequence of the polypeptide, but where the local order of amino acids has not been changed, and where the three dimensional architecture of the protein is conserved. For example, a circular permutant of a wild type 1000 amino acid polypeptide may have an N-terminal residue of residue number 500 (relative to the wild type protein), where residues 1-499 of the wild type protein are added the C-terminus. Such a circular permutant, relative to the wild type protein sequence would have, from N-terminus to C-terminus, amino acid numbers 500-1000 followed by 1-499, resulting in a circular permutant protein with amino acid 499 being the C-terminal residue. Thus, such an example circular permutant would have the same total number of amino acids as the wild type reference protein, and the amino acids would be in the same order locally in specific regions of the circular permutant, but the overall primary amino acid sequence is changed.

In some embodiments, an epigenetic editor comprises a circular permuted Cas protein, e.g. a circular permuted Cas9 protein. In some embodiments, the epigenetic editor comprises a fusion of a circular permuted Cas protein and an epigenetic effector domain, where the epigenetic effector domain is fused to the circular permuted Cas protein to a N-terminus or C-terminus that is different from that of wild type Cas protein.

In some embodiments, the circular permuted Cas protein comprises a N-terminal end of an N-terminal fragment of a wild type Cas protein fused to a C-terminus of a C-terminal fragment of the wild type Cas protein, hereby generating new N- and C-termini. Without wishing to be bound by any theory, the N-terminus and C-terminus of a wild type Cas protein may be locked in a small region, which may cause steric hinderance when the Cas protein is fused to an effect domain and reduced access to the target DNA sequence. In some embodiments, the epigenetic editor comprising a circular permutant Cas protein has reduced steric incompatibility as compared to an epigenetic editor comprising a wild type Cas protein counterpart. In some embodiments, the epigenetic editor comprising a circular permutant Cas protein has improved effectiveness as compared to an epigenetic editor comprising a wild type Cas protein counterpart. In some embodiments, the epigenetic editor comprising a circular permutant Cas protein has improved epigenetic editing accuracy as compared to an epigenetic editor comprising a wild type Cas protein counterpart. In some embodiments, the epigenetic editor comprising a circular permutant Cas protein has reduced off-target editing effect as compared to an epigenetic editor comprising a wild type Cas protein counterpart.

In some embodiments, the circular permutant Cas protein is a circular permutant Cas9 protein. In some embodiments, the circular permuted Cas9 protein includes an N-terminal fragment of a wild type Cas9 protein fused to the C-terminus of the Cas9 protein (e.g., in some cases via a linker, e.g., a cleavable linker), where the C-terminal amino acid of the N-terminal fragment (i.e., the C-terminus of the N-terminal fragment) includes an amino acid corresponding to amino acid 182D, 200P, 231G, 271Y, 311E, 1011G, 1017D, 1024K, 10291, 1030G, 1032A, 10421, 1245L, 1249P, 1250E, or 1283A of the wild type Cas9 protein sequence. In some cases, a circular permuted Cas9 protein includes an N-terminal fragment of a wild type Cas9 protein fused to the C-terminus of a C terminal fragment the wild type Cas9 protein (e.g., in some cases via a linker, e.g., a cleavable linker), where the N-terminal fragment includes an amino acid sequence corresponding to amino acids 1-182, 1-200, 1-231, 1-271, 1-311, 1-1011, 1-1017, 1-1024, 1-1029, 1-1030, 1-1032, 1-1042, 1-1245, 1-1249, 1-1250, or 1-1283 of the wild type Cas9 protein. Additional circular permuted Cas9 proteins as described in US Patent Application No. US20190233847 is incorporated herein by reference in its entirety.

### Guide Polynucleotides

In some embodiments, an epigenetic editor comprises a guide polynucleotide (or guide nucleic acid). For example, an epigenetic editor with a DNA binding domain that includes a CRISPR-Cas protein may also include a guide nucleic acid that is capable of forming a complex with the CRISPR-Cas protein.

Methods of using guide nucleotide sequence-programmable DNA-binding protein, such as Cas9, for site-specific DNA targeting (e.g., to modify a genome) are known in the art. The guide RNA (gRNA) may guide the programmable DNA binding protein, e.g a Class 2 Cas protein such as a Cas9 to a target sequence on a target nucleic acid molecule, where the gRNA hybridizes with and the programmable DNA binding protein and generates modification at or near the target sequence. In some embodiments, the gRNA and an epigenetic editor fusion protein may form a ribonucleoprotein (RNP), e.g., a CRISPR/Cas complex.

A guide nucleotide sequence, e.g. a guide RNA sequence, may comprises two parts: 1) a nucleotide sequence that shares homology to a target nucleic acid (e.g., and directs binding of a guide nucleotide sequence-programmable DNA-binding protein to the target); and 2) a nucleotide sequence that binds a nucleic acid guided programmable DNA-binding protein, for example, a CRISPR-Cas protein. The nucleotide sequence in 1) may comprise a spacer sequence that hybridizes with a target sequence. The nucleotide sequence in 2) may be referred to as a scaffold sequence of a guide nucleic acid, a tracrRNA, or an activating region of a guide nucleic acid, and may comprise a stem-loop structure. The scaffold sequences of guide nucleic acids as described in Jinek et al., Science 337:816-821(2012), U.S. Patent Application Publication US20160208288, and U.S. Patent Application Publication US20160200779 are each incorporated herein by reference in its entirety. A guide polynucleotide may be a single molecule or may comprise two separate molecules. For example, parts 1) and 2) as described above may be fused to form one single guide (e.g. a single guide RNA, or sgRNA), or may be two separate molecules. In some embodiments, a guide polynucleotide is a dual polynucleotides connected by a linker. In some embodiments, a guide polynucleotide is a dual polynucleotides connected by a non-nucleic acid linker, for example, a peptide linker or a chemical linker.

Methods for selecting, designing, and validating gRNAs and targeting sequences (or spacer sequences) are described herein and known to those skilled in the art. Software tools can be used to optimize the gRNAs corresponding to a target nucleic acid sequence, e.g., to minimize total off-target activity across the genome. For example, DNA sequence searching algorithm can be used to identify a target sequence in crRNAs of a gRNA for use with Cas9. Exemplary gRNA design tools, including as described in Bae, et al., Cas-OFFinder: A fast and versatile algorithm that searches for potential off-target sites of Cas9 RNA-guided endonucleases. Bioinformatics 30, 1473-1475 (2014)), is herein incorporated in its entirety.

A guide polynucleotide may be of variant lengths. In some embodiments, the length of the spacer or targeting sequence depends on the CRISPR/Cas component of the epigenetic editor system and components used. For example, different Cas proteins from different bacterial species have varying optimal targeting sequence lengths. Accordingly, the spacer sequence may comprise 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more than 50 nucleotides in length. In some embodiments, the spacer comprised 18-24 nucleotides in length. In some embodiments, the spacer comprises 19-21 nucleotides in length. In some embodiments, the spacer sequence comprises 20 nucleotides in length. In some embodiments, a guide nucleic acid (e.g., guide RNA) is from 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides that is complementary to a target sequence. In some embodiments, the target sequence is a DNA sequence. In some embodiments, the degree of complementarity between the targeting sequence of the gRNA and the target sequence on the target nucleic acid molecule is at least about 60%, 65%, 70%, 75%, 80%, 85%, 90%, 95%, 97%, 98%, 99%, or 100%. In some embodiments, the targeting sequence of the gRNA and the target sequence on the target nucleic acid molecule may be 100% complementary. In other embodiments, the targeting sequence of the gRNA and the target sequence on the target nucleic acid molecule may contain at least one mismatch. For example, the targeting sequence of the gRNA and the target sequence on the target nucleic acid molecule may contain 1, 2, 3, 4, 5, 6, 7, 8, 9, or 10 mismatches. In some embodiments, the target sequence is a sequence in the genome of a mammal. In some embodiments, the target sequence is a sequence in the genome of a human. In some embodiments, the 3′ end of the target sequence is immediately adjacent to a canonical PAM sequence (NGG). In some embodiments, the guide nucleic acid (e.g., guide RNA) is complementary to a sequence associated with a disease or disorder.

In some embodiments, a guide RNA is truncated. The truncation can comprise any number of nucleotide deletions. For example, the truncation can comprise 1, 2, 3, 4, 5, 10, 15, 20, 25, 30, 40, 50 or more nucleotides. In some embodiments, a guide polynucleotide comprises RNA. In some embodiments, a guide polynucleotide comprises DNA. In some embodiments, a guide polynucleotide comprises a mixture of DNA and RNA.

A guide polynucleotide may be modified. The modifications can comprise chemical alterations, synthetic modifications, nucleotide additions, and/or nucleotide subtractions. Modified nucleosides or nucleotides can be present in a gRNA. For example, a gRNA can comprise one or more non-naturally and/or naturally occurring components or configurations that are used instead of or in addition to the canonical A, G, C, and U residues. A modified RNA can include one or more of an alteration or a replacement, of one or both of the non-linking phosphate oxygens and/or of one or more of the linking phosphate oxygens in the phosphodiester backbone linkage, an alterations of the ribose sugar, e.g., of the 2′ hydroxyl on the ribose sugar (an exemplary sugar modification), an alteration of the phosphate moiety, a modification or replacement of a naturally occurring nucleobase, replacement or modification of the ribose-phosphate backbone, a modification of the 3′ end or 5′ end of the oligonucleotide, or replacement of a terminal phosphate group or conjugation of a moiety, cap, or linker, or any combination thereof.

In some embodiments, the ribose group (or sugar) may be modified. In some embodiments, modified ribose group may control oligonucleotide binding affinity for complementary strands, duplex formation, or interaction with nucleases. Examples of chemical modifications to the ribose group include, but are not limited to, 2′-O-methyl (2′-OMe), 2′-fluoro (2′-F), 2′-deoxy, 2′-O-(2-methoxyethyl) (2′-MOE), 2′-NH2, 2′-O-Allyl, 2′-O-Ethylamine, 2′-O-Cyanoethyl, 2′-O-Acetalester, or a bicyclic nucleotide such as locked nucleic acid (LNA), 2′-(5-constrained ethyl (S-cEt)), constrained MOE, or 2′-0,4′-C-aminomethylene bridged nucleic acid (2′,4′-BNANC). In some embodiments, 2′-O-methyl modification can increase binding affinity of oligonucleotides. In some embodiments, 2′-O-methyl modification can enhance nuclease stability of oligonucleotides. In some embodiments, 2′-fluoro modification can increase oligonucleotide binding affinity and nuclease stability.

In some embodiments, the phosphate group may be chemically modified. Examples of chemical modifications to the phosphate group includes, but are not limited to, a phosphorothioate (PS), phosphonoacetate (PACE), thiophosphonoacetate (thioPACE), amide, triazole, phosphonate, or phosphotriester modification. In some embodiments, PS linkage can refer to a bond where a sulfur is substituted for one nonbridging phosphate oxygen in a phosphodiester linkage, e.g., between nucleotides. An “s” may be used to depict a PS modification in gRNA sequences. In some embodiments, a gRNA or an sgRNA may comprise a phosphorothioate (PS) linkage at a 5′ end or at a 3′ end. In some embodiments, a gRNA or an sgRNA may comprise a phosphorothioate (PS) linkage at a 5′ end. In some embodiments, a gRNA or an sgRNA may comprise a phosphorothioate (PS) linkage at a 3′ end. In some embodiments, a gRNA or an sgRNA may comprise a phosphorothioate (PS) linkage at a 5′ end and at a 3′ end. In some embodiments, a gRNA or an sgRNA may comprise one, two, or three, or more than three phosphorothioate linkages at the 5′ end or at the 3′ end. In some embodiments, a gRNA or an sgRNA may comprise three phosphorothioate (PS) linkages at the 5′ end or at the 3′ end. In some embodiments, a gRNA or an sgRNA may comprise three phosphorothioate linkages at the 3′ end. In some embodiments, a gRNA or an sgRNA may comprise two and no more than two (i.e., only two) contiguous phosphorothioate (PS) linkages at the 5′ end or at the 3′ end. In some embodiments, a gRNA or an sgRNA may comprise three contiguous phosphorothioate (PS) linkages at the 5′ end or at the 3′ end. In some embodiments, a gRNA or an sgRNA may comprise the sequence 5′-UsUsU-3′ at the 3′end or at the 5′ end, wherein U indicates a uridine and wherein s indicates a phosphorothioate (PS) linkage. In some embodiments, the nucleobase may be chemically modified. Examples of chemical modifications to the nucleobase include, but are not limited to, 2-thiouridine, 4-thiouridine, N6-methyladenosine, pseudouridine, 2,6-diaminopurine, inosine, thymidine, 5-methylcytosine, 5-substituted pyrimidine, isoguanine, isocytosine, or halogenated aromatic groups. Chemical modifications can be made at a part of a guide polynucleotide or the entire guide polynucleotide. In some embodiments, a total of 1, 2, 3, 4, 5, 6, 7, 8, 9, 10, 11, 12, 13, 14, 15, 16, 17, 18, 19, 20, 21, 22, 23, 24, or 25 base pairs of a guide RNA are chemically modified. In some embodiments, a total of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs of a guide RNA are chemically modified. In some embodiments, a total of 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150 base pairs of a guide RNA are chemically modified. Chemical modifications can be made in the protospacer region, the tracr RNA, the crRNA, the stem loop, or any combination thereof.

### Zinc Finger Proteins

In some embodiments, an epigenetic editor described herein comprises a nucleic acid binding domain comprising a zinc finger domain.

Zinc finger proteins are DNA-binding proteins that contain one or more zinc fingers. In some embodiments, a zinc finger (ZF) comprises a relatively small polypeptide domain comprising approximately 30 amino acids. A zinc finger may comprise an α-helix adjacent an antiparallel β-sheet (known as a ββα-fold) which may co-ordinate with a zinc ion between four Cys and/or His residues, as described further below. In some embodiments, a ZF domain recognizes and binds to a nucleic acid triplet, or an overlapping quadruplet, in a double-stranded DNA target sequence. In certain embodiments, ZFs may also bind RNA and proteins.

As used herein, the term “zinc finger” (ZF) or “zinc finger motif” (ZF motif) refers to an individual “finger”, which comprises a beta-beta-alpha (ββα)-protein fold stabilized by a zinc ion as described elsewhere herein. In some embodiments, each finger includes approximately 30 amino acids. In some embodiments, ZF proteins or ZF protein domains are protein motifs that contain multiple fingers or finger-like protrusions that make tandem contacts with their target molecule. For example, a ZF finger may bind a triplet or (overlapping) quadruplet nucleotide sequence. Accordingly, a tandem array of ZF fingers may be designed for ZF proteins that do not naturally exist to bind desired targets.

Zinc finger proteins are widespread in eukaryotic cells. An exemplary motif characterizing one class of these proteins (C2H2 class) is -Cys-(X)2-4-Cys-(X)12-His-(X)3-5His (SEQ ID NO: 1158), where X is any amino acid. A single finger domain may be about 30 amino acids in length. In some embodiments, a single finger comprises an alpha helix containing the two invariant histidine residues co-ordinated through zinc with the two cysteines of a single beta turn.

In some embodiments, amino acid sequence of a zinc finger protein, e.g. a Zif268 protein may be altered by making amino acid substitutions at the helix positions (e.g., positions—1, 2, 3 and 6 of Zif268) on a zinc finger recognition helix. For example, modified zinc fingers with non-naturally occurring DNA recognition specificity may be generated by phage display and combinatorial libraries with randomized side-chains in either the first or middle finger of a Zif268 and then isolated with an altered Zif268 binding site in which the appropriate DNA sub-site was replaced by an altered DNA triplet.

In some embodiments, a zinc finger comprises a C2H2 finger. In some embodiments, a zinc finger protein comprises a ZF array that comprises sequential C2H2-ZFs each contacting three or more sequential bases. In some embodiments, Zinc finger protein structures, for example, zinc finger protein Zif268 and its variants bound to DNA show a semi-conserved pattern of interactions, in which typically three amino acids from the alpha-helix of the zinc finger contact three adjacent base pairs in the DNA. Accordingly, in embodiments, zinc finger DNA-binding domains function in a modular manner with a one-to-one interaction between a zinc finger and a three-base-pair tri-nucleotide sequence in a DNA sequence.

In some embodiments, an epigenetic editor comprises a zinc finger motif comprising of a sequence: N′--(Helix 1)- -(Helix 2)- -(Helix 3)- -(Helix 4)--(Helix 5)- -(Helix 6)- -C′, wherein the (Helix) is a-six contiguous amino acid residue peptide that forms a short alpha helix. In some embodiments, an epigenetic editor comprises a zinc finger motif comprising of a sequence: N′--(Helix 1)- -(Helix 2)- -(Helix 3)- -(Helix 4)--(Helix 5)-- -C′, wherein the (Helix) is a-six contiguous amino acid residue peptide that forms a short alpha helix.

In some embodiments, two or more zinc fingers are linked together in a tandem array to achieve specific recognition and binding of a contiguous DNA sequence. Zinc finger or zinc finger arrays in an epigenetic editor may be naturally occurring, or may be artificially engineered for desired DNA binding specificity. For example, DNA binding characteristics of individual zinc fingers may be engineered by randomizing the amino acids at the alpha-helical positions of the zinc fingers involved in DNA binding and using selection methodologies such as phage display to identify desired variants capable of binding to DNA target sites of interest.

Engineered zinc finger binding domain can have a novel binding specificity as compared to a naturally-occurring zinc finger protein. Zinc fingers with desired DNA binding specificity can be designed and selected via various approaches. For example, databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence may be used to design zinc finger arrays for specific DNA sequences. See, for example, U.S. Pat. Nos. 6,453,242, 6,534,261, and 8,772,453, incorporated by reference herein in their entirety. In some embodiments, a zinc finger array may be designed and selected from a library of zinc fingers, e.g., a randomized zinc finger library. In some embodiments, a zinc finger with novel DNA binding specific is generated by selection-based methods on combinatorial libraries. For example, a zinc finger can be selected with phage display which involves displaying zinc finger proteins on the surface of filamentous phage, followed by sequential rounds of affinity selection with biotinylated target DNA to enrich for phage expressing proteins able to bind the specific target sequence. Bacterial-two-hybrid (B2H) system may also be used for selection of zinc fingers that bind specific target sites from randomized libraries. For example, a zinc finger binding site may be placed upstream of a weak promoter driving expression of two selectable markers in host cells, e.g. E. coli cells. A library of zinc fingers, fused to a fragment of the reporter protein, e.g. a yeast Gal11P protein, can be expressed in the cells and binding of a zinc finger to the target site recruits an RNA polymerase-Gal4 fusion, thus activating transcription and allowing survival of the cells on selective medium. Rational design and selection of zinc fingers as described in Maeder et al., 2008, Mol. Cell, 31:294-301; Joung et al., 2010, Nat. Methods, 7:91-92; Isalan et al., 2001, Nat. Biotechnol., 19:656-660, Rebar, et al., Science 263, 671-673 (1994), and Joung, et al. Proc Natl Acad Sci USA 97, 7382-7387 (2000), each of which incorporated herein by reference in its entirety.

In some embodiments, zinc fingers may be evolved and selected with a continuous evolution system (PACE) comprising a host cell, e.g. a E. coli cell, a “helper phagemid” present in all host cells and encoding all phage proteins except one phage protein (e.g. a g3p protein), an “accessory plasmid”, present in all host cells, that expresses the g3p protein in response to an active library member; and a “selection phagemid” expressing the library of proteins or nucleic acids being evolved, which is replicated and packaged into secreted phage particles. Helper and accessory plasmids can be combined into a single plasmid. New host cells can only be infected by phage particles that contain g3p. Fit selection phagemids encode library members that induce g3p expression from the accessory plasmid can be packaged into phage particles that contain g3p. g3p containing phage particles can infect new cells, leading to further replication of the fit selection phagemids, while g3p-deficient phage particles are non-infectious, and therefore low-fitness selection phagemids cannot propagate. The selection system, in combination with a continuous flow of host cells through a lagoon that permits replication of the phagemid but not the host cells, may be used to rapidly select zinc fingers. PACE system as described in U.S. Pat. No. 9,023,594 is incorporated by reference in its entirety.

A zinc finger DNA binding domain of an epigenetic editor may include one or multiple zinc fingers. For example, a zinc finger DNA binding domain may include 2, 3, 4, 5, 6, 7, 8, 9, 10, 11, 12 or more zinc fingers. In some embodiments, a zinc finger DNA binding domain has at least three zinc fingers. In some embodiments, a zinc finger DNA binding domain has at least 4, 5, or 6 zinc fingers. In some embodiments, a zinc finger DNA binding domain has three zinc fingers. In some embodiments, a zinc finger DNA binding domain has at least two zinc fingers. In some embodiments, a zinc finger DNA binding domain has an array of two-finger units.

A zinc finger DNA binding domain of an epigenetic editor may be designed for optimized specificity. In some embodiments, a sequential selection strategy is used to design a multi-finger ZF domain. For example, in a multi-finger ZF domain, a first finger may be randomized and selected with phage display, a small pool of selected fingers may be carried into the next stage, in which the second finger is randomized and selected. The process may be repeated multiple times depending on the number of fingers in the ZF domain. In some embodiments, a parallel optimization is used to design a multi-finger ZF domain. For example, a master randomized library may be interrogated using a B2H system under low selection stringency to identify a variety of individual fingers capable of binding each 3 base pair sub-site of the target site. The three selected populations may then be randomly shuffled to generate a library of multi-finger proteins, which may subsequently be interrogated under high-stringency selection conditions to identify three-finger proteins targeted to a specific nine base pair site. In additional embodiments, a large number of low-stringency selections may be used to generate a master library of single fingers, from which multi-finger proteins, e.g., three finger ZF proteins may be selected. For example, a master library or an archive may include pre-selected zinc finger pools each containing a mixture of fingers targeted to a different three base pair subsite of DNA sequences at a defined position within a three finger ZF protein. In certain embodiments, a zinc finger archive comprises at least 192 finger pools (64 potential three bp target subsites for each position in a three-finger protein). In some embodiments, a zinc finger archive comprises at least a zinc finger pool comprises at least at least 10, 20, 30, 40, 50, 60, 70, 80, 90, 95, 100 or more different fingers. In some embodiments, a smaller library is created form the archive for interrogation with a reporting system, e.g., a bacterial two-hybrid selection system.

In some embodiments, a multiple-finger ZF domain, e.g., a three-finger ZF domain may be designed and selected using two complementary libraries. For example, a three-finger ZF domain may be designed with two pre-made zinc finger phage-display libraries, where the first library contains randomized DNA-binding amino acid positions in fingers 1 and 2, and a second library contains randomized DNA-binding amino acid positions in fingers 2 and 3. The two libraries are complementary because the first library contains randomizations in all the base-contacting positions of finger 1 and certain base-contacting positions of finger 2, whereas the second library contains randomizations in the remaining base-contacting positions of finger 2 and all the base-contacting positions of finger 3. Selections of “one-and-a-half” fingers from each master library may be carried out in parallel using DNA sequences in which five nucleotides have been fixed to a sequence of interest. Subsequently, zinc finger encoding sequences may be amplified from the recovered phage using PCR, and sets of “one-and-a-half” fingers can be paired to yield recombinant three-finger DNA-binding domains.

In some embodiments, a multi-finger ZF domain may be designed depending on the context effects of adjacent fingers. In some embodiments, a multi-finger ZF domain is designed and without selection. For example, a three-finger ZF domain may be assembled using N-terminal and C-terminal fingers identified in other arrays containing a common middle finger, using libraries containing an archive of three-finger ZF arrays comprising pre-selected and/or tested three-finger arrays.

Software for designing and selecting ZF arrays, for example, ZiFit (http://bindr.gdcb.iastate.edu/ZiFiT/; http://www.zincfingers.org/software-tools.htm) are available and known to those skilled in the art.

Accordingly, a zinc finger DNA binding domain of an epigenetic editor may include one or multiple zinc fingers. For example, a zinc finger DNA binding domain may include 2, 3, 4, 5, 6, 7, 8, 9, 10, 11, 12 or more zinc fingers. In some embodiments, a zinc finger DNA binding domain has at least three zinc fingers. In some embodiments, a zinc finger DNA binding domain has at least 4, 5, or 6 zinc fingers. In some embodiments, a zinc finger DNA binding domain has three zinc fingers. In some embodiments, a zinc finger DNA binding domain comprising at least three zinc fingers recognizes a target DNA sequence of 9 or 10 nucleotides. In some embodiments, a zinc finger DNA binding domain comprising at least four zinc fingers recognizes a target DNA sequence of 12 to 14 nucleotides. In some embodiments, a zinc finger DNA binding domain comprising at least six zinc fingers recognizes a target DNA sequence of 18 to 21 nucleotides.

In some embodiments, an epigenetic editor as disclosed herein comprises non-natural and suitably contain 3 or more zinc fingers. In some embodiments, an epigenetic editor comprises 4, 5, 6, 8, 9, 10, 11, 12, 13, 14, 15, 16, 17, 18 or more (e.g. up to approximately 30 or 32) zinc fingers motifs arranged adjacent one another in tandem, forming arrays of ZF motifs. In some embodiments, an epigenetic editor includes at least 3 ZF motifs, at least 4 ZF motifs, at least 5 ZF motifs, or at least 6 ZF motifs, at least 7 ZF motifs, at least 8 ZF motifs, at least 9 ZF motifs, at least 10 ZF motifs, at least 11 or at least 12 ZF motifs in the nucleic acid binding domain. In some embodiments, an epigenetic editor includes up to 6, 7, 8, 10, 11, 12, 16, 17, 18, 22, 23, 24, 28, 29, 30, 34, 35, 36, 40, 41, 42, 46, 47, 48, 54, 55, 56, 58, 59, or 60 ZF motifs in the nucleic acid binding domain.

In some embodiments, a zinc finger or zinc finger array targeting a specific DNA sequence is designed with a modular assembly approach. For example, two or more pre-selected zinc fingers may be fused in a tandem fashion.

In some embodiments, a zinc finger array comprises multiple zinc fingers fused via peptide bonds. In some embodiments, a zinc finger array comprises multiple zinc fingers, one or more of which connected by peptide linkers. For example, zinc fingers in a multiple finger array can be linked by peptide linkers of 3, 4, 5, 6, 7, 8, 9, 10, 11, 12, 13, 14, 15 or more amino acids in length. In some embodiments, zinc fingers in a multiple finger array are linked by peptide linkers of 5 amino acids in length. In some embodiments, zinc fingers in a multiple finger array are linked by peptide linkers of 6 amino acids in length. In some embodiments, the two-finger units bind adjacent bases and are connected by a linker with the sequence TGSQKP (SEQ ID NO.: 704). In some embodiments the two-finger units bind sequences that are separated by 1 or 2 nucleotides and the two-finger units are separated by a linker with the sequence TGGGGSQKP (SEQ ID NO.: 705).

In some embodiments, ZF-containing proteins may contain ZF arrays of 2 or more ZF motifs, which may be directly adjacent one another (i.e. separated by a short (canonical) linker sequence), or may be separated by longer, flexible or structured polypeptide sequences. In some embodiments, directly adjacent fingers bind to contiguous nucleic acid sequences, i.e. to adjacent trinucleotides/triplets. In some embodiments, adjacent fingers cross-bind between each other's respective target triplets, which may help to strengthen or enhance the recognition of the target sequence, and leads to the binding of overlapping quadruplet sequences. In some embodiments, distant ZF domains within the same protein may recognize (or bind to) non-contiguous nucleic acid sequences or even to different molecules (e.g. protein rather than nucleic acid).

In some embodiments, an epigenetic editor comprises zinc fingers comprising more than 3-fingers. In some embodiments, an epigenetic editor comprises at least 6 zinc fingers in the DNA binding domain. In some embodiments, an epigenetic editor comprises 6 zinc fingers in the DNA binding domain that binds to a 18 bp target sequence. In some embodiments, the 18 bp target sequence is unique in the human genome. In some embodiments, an epigenetic editor comprises zinc fingers comprising at least 7, 8, 9, 10, 11, 12, 13, 14, 15 or more zinc fingers. In some embodiments, the strong affinity of three-finger proteins would allow subsets of the longer array to bind DNA and therefore decrease specificity. Without wishing to be bound by any theory, zinc finger proteins comprising multiple two-finger units or three-finger units joined by extended linkers may confer higher DNA binding specificity as compared to fewer fingers, or an array with same number of fingers simply joined via peptide bonds. In some embodiments, an epigenetic editor comprises at least three two-finger units connected by peptide linkers, where each of the two finger units binds a subsite in the target DNA sequence. In some embodiments, an epigenetic editor comprises at least four two-finger units connected by peptide linkers, wherein each of the two finger units binds a subsite in the target DNA sequence. In some embodiments, an epigenetic editor comprises at least five two-finger units connected by peptide linkers, wherein each of the two finger units binds a subsite in the target DNA sequence. In some embodiments, an epigenetic editor comprises at least six, seven, eight, nine, ten, or more two-finger units connected by peptide linkers, wherein each of the two finger units binds a subsite in the target DNA sequence. In some embodiments, an epigenetic editor comprises at least two three-finger units connected by peptide linkers, where each of the three finger units binds a subsite in the target DNA sequence. In some embodiments, an epigenetic editor comprises at least three three-finger units connected by peptide linkers, where each of the three finger units binds a subsite in the target DNA sequence. In some embodiments, an epigenetic editor comprises at least four three-finger units connected by peptide linkers, wherein each of the three finger units binds a subsite in the target DNA sequence. In some embodiments, an epigenetic editor comprises at least five three-finger units connected by peptide linkers, wherein each of the three finger units binds a subsite in the target DNA sequence. In some embodiments, an epigenetic editor comprises at least six, seven, eight, nine, ten, or more three-finger units connected by peptide linkers, wherein each of the three finger units binds a subsite in the target DNA sequence.

In some embodiments, multiple zinc fingers, each recognizing three specific DNA nucleotides, or trinucleotide “subsites”, are assembled to target specific DNA sequences in target genes. In some embodiments, such DNA subsites are contiguous sequences in a target gene. In some embodiments, one or more of the DNA subsites are separated by gaps in the target gene. for example, a multi-finger ZF may recognize DNA subsites that span a 1, 2, 3 or more base pairs of inter-subsite gaps between adjacent subsites. In some embodiments, zinc fingers in the multi-finger ZF are connect via peptide linkers. The peptide linkers may be of 2, 3, 4, 5, 6, 7, 8, 9, 10, 11, 12, 13, 14, 15, 16, 17, 18, 19, 20, or more amino acids in length. In some embodiments, a linker comprises 5 or more amino acids. In some embodiments, a linker comprises 7-17 amino acids. In some embodiments, the linker is a flexible linker. In some embodiments, the linker is a rigid linker, e.g., a linker comprising one or more Prolines.

Zinc finger arrays with sequence specific DNA binding activity may be fused to functional effector domains, e.g. epigenetic effector domains as described herein to confer epigenetic modifications to DNA sequences, or associated histones in a target gene. In some embodiments, an epigenetic editor described herein comprises a zinc finger array having specificity for a target DNA sequence. In some embodiments a zinc finger array may have the sequence:

(SEQ ID NO.: 1157)
SRPGERPFQCRICMRNFSNNNNNNNHTRTHTGEKPFQCRICMRNFSNNN
NNNNHLRTH[linker]FQCRICMRNFSNNNNNNNHTRTHTGEKPFQCR
ICMRNFSNNNNNNNHLRTH[linker]FQCRICMRNFSNNNNNNNHTRT
HTGEKPFQCRICMRNFSNNNNNNNHLRTHLRGS.

Where NNNNNNN represents the amino acids of the zinc finger recognition helix, which confer DNA-binding specificity upon the zinc finger. And [linker] represents a linker sequence. In some embodiments the linker sequence may be TGSQKP (SEQ ID NO.: 704). In some embodiments the linker sequence may be TGGGGSQKP (SEQ ID NO.: 705). In some embodiments, the two linkers of the zinc finger array are the same. In some embodiments, the two linkers of the zinc finger array are different.

In some embodiments, the programmable DNA binding protein comprises an argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of −24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 2016 July; 34(7):768-73. PubMed PMID: 27136078; Swarts et al., Nature. 507(7491) (2014):258-61; and Swarts et al., Nucleic Acids Res. 43(10) (2015):5120-9, each of which is incorporated herein by reference.

In some embodiments, the nucleic acid binding domain comprises a virus derived RNA-binding domain guided by an RNA sequence to bind the target gene. In some embodiments, the nucleic acid binding domain comprises a K Homology (KH) domain, a MS2 coat protein domain, a PP7 coat protein domain, a SfMu Com coat protein domain, a sterile alpha motif, a telomerase Ku binding motif and Ku protein, a telomerase Sm7 binding motif and Sm7 protein, or any other RNA recognition motifs.

In some embodiments, the nucleic acid binding domain comprises an inactivated nuclease, for example, an inactivated meganuclease. Additional non-limiting examples of DNA binding domains include tetracycline-controlled repressor (tetR) DNA binding domain, leucine zippers, helix-loophelix (HLH) domains, helix-turn-helix domains, zinc fingers, R-sheet motifs, steroid receptor motifs, bZIP domains homeodomains, and AT-hooks.

### Effector Domains

Epigenetic editors or epigenetic editing complexes provided herein may include one or more effector protein domains that modulate expression of a target gene. An effector domain can be used to contact a target polynucleotide sequence in a target gene to effect an epigenetic modification, for example, a change in methylation state of DNA nucleotides in the target gene. Accordingly, an epigenetic editor with one or more effector domains may provide the effect of modulating expression of a target gene without altering the DNA sequence of the target gene. For example, in some embodiments, an effector domain results in repression or silencing of expression of a target gene. In some embodiments, an effector domain results in activation or increased expression of a target gene.

In an aspect, the epigenetic modification described herein is sequence specific, or allele specific. For example, an epigenetic editor may specifically target a DNA sequence recognized by a DNA binding domain of the epigenetic editor. In some embodiments, the target DNA sequence is specific to one copy of a target gene. In some embodiments, the target gene sequence is specific to one allele of a target gene. Accordingly, the epigenetic modification and modulation of expression thereof may be specific to one copy or one allele of the target gene. For example, an epigenetic editor may repress or activate expression of a specific copy harboring a target sequence recognized by the DNA binding domain. In some embodiments, the epigenetic editor represses expression of a specific copy of a target gene, wherein the copy is associated with a disease or disorder. In some embodiments, the epigenetic editor represses expression of a specific copy of a target gene, wherein the copy harbors a mutation associated with a disease or disorder. In some embodiments, the epigenetic editor activates expression of a specific copy of a target gene. In some embodiments, the epigenetic editor activates expression of a specific copy of a target gene that is a wild type copy. The epigenetic modification mediated by an epigenetic editor may be in the vicinity of the target gene, or may be distal to the target gene. In some embodiments, an epigenetic editor may initiate a chemical modification, e.g, DNA methylation, in one or more nucleotides of the target gene. Such methylation may be initiated near the target sequence, and may subsequently spread to one or more nucleotides in the target gene distant from the target sequence.

An epigenetic effector may deposit a chemical modification at the chromatin at the position of a target gene. Non limiting examples of chemical modifications include methylation, demethylation, acetylation, deacetylation, phosphorylation, SUMOylation and/or ubiquitination of the DNA or histone residues of the chromatin. In some embodiments, an epigenetic effector may make histone tail modifications. In some embodiments epigenetic effectors may add or remove active marks on histone tails. In some embodiments the active marks may include H3K4 methylation, H3K9 acetylation, H3K27 acetylation, H3K36 methylation, H3K79 methylation, H4K5 acetylation, H4K8 acetylation, H4K12 acetylation, H4K16 acetylation, and/or H4K20 methylation. In some embodiments epigenetic effectors may add or remove repressive marks on histone tails. In some embodiments these repressive marks may include H3K9 methylation and/or H3K27 methylation.

In some embodiments, an effector domain in an epigenetic editor alters a chemical modification state of a target gene harboring a target sequence. For example, an effector domain may alter a chemical modification state of a nucleotide in the target gene. In some embodiments, an effector domain of an epigenetic editor deposits a chemical modification at a nucleotide in the target gene. In some embodiments, an effector domain of an epigenetic editor deposits a chemical modification of a histone associated with the target gene. In some embodiments, an effector domain of an epigenetic editor removes a chemical modification at a nucleotide in the target gene. In some embodiments, an effector domain of an epigenetic editor removes a chemical modification of a histone associated with the target gene. In some embodiments, the chemical modification increases expression of the target gene. For example, the epigenetic editor may comprise an effector domain having histone acetyltransferase activity. In some embodiments, the chemical modification decreases expression of the target gene. For example, the epigenetic editor may comprise an effector domain having DNA methyltransferase activity.

The chemical modifications may be deposited or removed by the epigenetic editor in any region of a target gene. In some embodiments, the chemical modification is deposited or removed at a single nucleotide. In some embodiments, the chemical modification is deposited or removed at a single histone. In some embodiments, the chemical modification is deposited at more than 10, 20, 30, 40, 50, 60, 70, 80, 90, 100, 200, 300, 400, 500, 600, 700, 800, 900, 1000, 1500, 2000, 2500, 3000 or more nucleotides. In some embodiments, the chemical modification is removed from more than 10, 20, 30, 40, 50, 60, 70, 80, 90, 100, 200,300, 400, 500, 600, 700, 800, 900, 1000, 1500, 2000, 2500, 3000 or more nucleotides. In some embodiments, the effector domain of an epigenetic editor alters a chemical modification in a nucleotide in a promoter region of the target gene. In some embodiments, the effector domain of an epigenetic editor alters a chemical modification in at least 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 1500, 2000, 2500, 3000 or more nucleotides in a promoter region of the target gene. In some embodiments, the effector domain of an epigenetic editor alters a chemical modification in a nucleotide in a enhancer region of the target gene. In some embodiments, the effector domain of an epigenetic editor alters a chemical modification in at least 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 1500, 2000, 2500, 3000 or more nucleotides in a enhancer region of the target gene. In some embodiments, the effector domain of an epigenetic editor alters a chemical modification in a nucleotide in a coding region of the target gene. In some embodiments, the effector domain of an epigenetic editor alters a chemical modification in at least 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 1500, 2000, 2500, 3000 or more nucleotides in a coding region of the target gene. In some embodiments, the effector domain of an epigenetic editor alters a chemical modification in a nucleotide in an exon of the target gene. In some embodiments, the effector domain of an epigenetic editor alters a chemical modification in at least 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 1500, 2000, 2500, 3000 or more nucleotides in an exon of the target gene. In some embodiments, the effector domain of an epigenetic editor alters a chemical modification in a nucleotide in an intron of the target gene. In some embodiments, the effector domain of an epigenetic editor alters a chemical modification in at least 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 1500, 2000, 2500, 3000 or more nucleotides in an intron of the target gene. In some embodiments, the effector domain of an epigenetic editor alters a chemical modification in a nucleotide in an insulator region of the target gene or chromosome. In some embodiments, the effector domain of an epigenetic editor alters a chemical modification in at least 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 1500, 2000, 2500, 3000 or more nucleotides in an insulator region of the target gene or chromosome. In some embodiments, the effector domain of an epigenetic editor alters a chemical modification in a nucleotide in a silencer region of the target gene or chromosome. In some embodiments, the effector domain of an epigenetic editor alters a chemical modification in at least 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 1500, 2000, 2500, 3000 or more nucleotides in a silencer region of the target gene or chromosome. In some embodiments, the chemical modification is altered at a CTCF binding region of a target gene or chromosome. In some embodiments, the alteration of the chemical modification state is at or near a transcription initiation site (TSS). In some embodiments, the alteration of the chemical modification state is 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, or 1000, 1500, 2000, 2500, 3000 nucleotides upstream of a TSS. In some embodiments, the alteration of the chemical modification state is 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 1000, 1500, 2000, 2500, 3000 nucleotides flanking a TSS. In some embodiments, the alteration of the chemical modification state is a DNA methylation state, for example, methylation of DNA near TSS by an epigenetic editor comprising an effector domain with DNA methyltransferase activity, thereby reducing or silencing expression of the target gene.

The epigenetic modification mediated by an epigenetic editor may be in the vicinity of the target gene, or may be distant to the target gene, or spread from an initial epigenetic modification initiated by the epigenetic editor at one or more nucleotides in a target sequence of the target gene. For example, an epigenetic editor may initiate a chemical modification, e.g, DNA methylation, in one or more nucleotides of the target gene. Such methylation may be initiated near the target sequence, and may subsequently spread to one or more nucleotides in the target gene distant from the target sequence. In some embodiments, the epigenetic editor places, deposits, or removes a modification at a single nucleotide in a target sequence in the target gene, which subsequently spreads to one or more nucleotides upstream or downstream of the single nucleotide. In some instances, additional proteins or transcription factors, for example, transcription repressors, methyltransferases, or transcription regulation scaffold proteins, are involved in the spreading of the chemical modification. In some instances, distant modification is solely mediated by the epigenetic editor. In some embodiments, the chemical modification mediated by an epigenetic editor is 50, 100, 150, 200, 250, 300, 350, 400, 450, or 500 nucleotides from the epigenetic editing target sequence. In some embodiments, the chemical modification mediated by an epigenetic editor is 50, 100, 150, 200, 250, 300, 350, 400, 450, or 500 nucleotides upstream of the epigenetic editing target sequence. In some embodiments, the chemical modification mediated by an epigenetic editor is 50, 100, 150, 200, 250, 300, 350, 400, 450, or 500 nucleotides downstream of the epigenetic editing target sequence. In some embodiments, the chemical modification mediated by an epigenetic editor is at least 500, 600, 700, 800, 900, 1000, 1100, 1200, 1300, 1400, 1500, 1600, 1700, 1800, 1900, 2000 or more nucleotides from the epigenetic editing target sequence. In some embodiments, the chemical modification mediated by an epigenetic editor is at least 500, 600, 700, 800, 900, 1000, 1100, 1200, 1300, 1400, 1500, 1600, 1700, 1800, 1900, 2000 or more nucleotides upstream of the epigenetic editing target sequence. In some embodiments, the chemical modification mediated by an epigenetic editor is at least 500, 600, 700, 800, 900, 1000, 1100, 1200, 1300, 1400, 1500, 1600, 1700, 1800, 1900, 2000 or more nucleotides downstream of the epigenetic editing target sequence.

Chemical modifications that may be deposited or removed from a target gene or chromosome region include, but are not limited to DNA or histone methylation, de-methylation, acetylation, deacetylation, phosphorylation, ubiquitination, or any combination thereof.

In some embodiments, the alteration of the chemical modification state is a DNA methylation state. For example, methylation can be introduced by an effector domain having DNA methyltransferase activity, or can be removed by an effector domain having DNA-demethylase activity. In some embodiments, alteration in methylation state mediated by an epigenetic effector is at a CpG dinucleotide sequence in the target gene or chromosome. In some embodiments, alteration in methylation state mediated by an epigenetic effector is at 1, 2, 3, 4, 5, 6, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, or 1000 CpG dinucleotide sequences in the target gene or chromosome. In some embodiments, the CpG dinucleotide sequences are methylated. In some embodiments, the CpG dinucleotide sequences are de-methylated. In some embodiments, CpG dinucleotide sequences methylated by the epigenetic editor are within target gene or chromosome regions known as CpG islands. In some embodiments, the CpG dinucleotide sequences methylated by the epigenetic editor are not in a CpG island. A CpG island generally refers to a nucleic acid sequence or chromosome region that comprises high frequency of CpG dinucleotides. For example, a CpG island may comprise at least 50% of GC content. In embodiments, a CpG island has a high of observed-to-expected CpG ratio, for example, an observed-to-expected CpG ratio of at least 60%. As used herein, observed-to-expected CpG ratio is determined by Number of CpG*(sequence length)/(Number of C*Number of G). In some embodiments, the CpG island has an observed-to-expected CpG ratio of at least 60%, 70%, 80%, 90% or more. In some embodiments, the CpG island is a sequence or region of at least 200 nucleotides. In some embodiments, the CpG island is a sequence or region of at least 250 nucleotides. In some embodiments, the CpG island is a sequence or region of at least 300 nucleotides. In some embodiments, the CpG island is a sequence or region of at least 350 nucleotides. In some embodiments, the CpG island is a sequence or region of at least 400 nucleotides. In some embodiments, the CpG island is a sequence or region of at least 450 nucleotides. In some embodiments, the CpG island is a sequence or region of at least 500 nucleotides. In some embodiments, the CpG island is a sequence or region of at least 550 nucleotides. In some embodiments, the CpG island is a sequence or region of at least 550, at least 600, at least 650, at least 700, at least 750, at least 800 or more nucleotides. In some embodiments, only 1, 2, 3, 4, 5, 6, 6, 7, 8, 9, 10, 11, 12, 13, 14, 15, 16, 17, 18, 19, 20, 30, 40, or less than 50 CpG dinucleotides are methylated by the epigenetic editor. In some embodiments, CpG dinucleotide sequences de-methylated by the epigenetic editor are within target gene or chromosome regions known as CpG islands. In some embodiments, the CpG dinucleotide sequences de-methylated by the epigenetic editor are not in a CpG island. In some embodiments, only 1, 2, 3, 4, 5, 6, 6, 7, 8, 9, 10, 11, 12, 13, 14, 15, 16, 17, 18, 19, 20, 30, 40, or less than 50 CpG dinucleotides are de-methylated by the epigenetic editor. In some embodiments, sequence within about 3000 base pairs of the target sequence are methylated by the epigenetic editor. In some embodiments, sequences that is within about 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, or 100 base pairs of the target sequence are methylated by the epigenetic editor.

In some embodiments, the alteration of chemical modification, e.g., methylation, is at a hypomethylated nucleic acid sequence. For example, the chemically modified sequence in the target gene or chromosome region may lack methyl groups on the 5-methyl cytosine nucleotide (e.g., in CpG) as compared to a standard control. Hypomethylation may occur, for example, in aging cells or in cancer (e.g., early stages of neoplasia) relative to the younger cell or non-cancer cell, respectively. In some embodiments, the target polynucleotide sequence is within a CpG island. In some embodiments, the target gene is known to be associated with a disease or condition. In some embodiments, the target gene comprises a specific copy of disease related sequence. In some embodiments, the target gene harbors the target sequence which is related to a disease.

In some embodiments, the alteration of chemical modification, e.g., methylation, is at a hypermethylated nucleic acid sequence. In some embodiments, the chemical modification is within a CpG island.

Chromatin or DNA sequences chemically modified in the target gene may be within or near the target sequence recognized by an epigenetic editor. In some embodiments, DNA sequence within about 3000 base pairs of the target nucleic acid sequence is chemically modified, e.g., methylated, by the epigenetic editor. In some embodiments, DNA sequence within about 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, or 100 base pairs of the target nucleic acid sequence is chemically modified by the epigenetic editor.

In some embodiments, chemical modification, e.g. methylation or demethylation, may be introduced by the epigenetic editor in a target gene where the modification isn't at a CpG dinucleotide. For example, the target gene sequence may be de-methylated at the C nucleotide of CpA, CpT, or CpC sequences. Without wishing to be bound by any theory, DNMT3A may be able to methylate nucleotides at non-CpG sites. In some embodiments, an epigenetic editor comprises a DNMT3A domain and effects methylation at CpG, CpA, CpT, and/or CpC sequences. In some embodiments, an epigenetic editor comprises a DNMT3A domain that lacks a regulatory subdomain and only maintains a catalytic domain. In some embodiments, the epigenetic editor comprising a DNMT3A with catalytic domain only effects methylation exclusively at CpG sequences. In some embodiments, an epigenetic editor comprises a DNMT3A domain comprises a mutation, e.g. a R836A mutation, has higher methylation activity at CpA, CpC, and/or CpT sequences as compared to an epigenetic editor comprising a wild type DNMT3A domain.

In some embodiments, the effector domain comprises a transcription related protein. For example, the effector domain may comprise a transcription factor, a transcription activator, or a transcription repressor. In some embodiments, the effector domain in an epigenetic editor recruits one or more transcription related proteins to a target gene that harbors a target sequence. For example, the effector domain may recruit a transcription factor, a transcription activator, or a transcription repressor to the target gene harboring the target sequence. In some embodiments, the transcription related proteins are endogenous. In some embodiments, the transcription related proteins are introduced together or sequentially with the epigenetic editor. In some embodiments, the transcription related protein is recruited to a region of the target gene in close proximity to the target sequence. In some embodiments, the transcription related protein is recruited to a region that is 100-200 bp, 200-300 bp, 300-400 bp, 400-500 bp, 500-600 bp, 600-700 bp, 700-800 bp, 800-900 bp, 900-1000 bp or more 5′ to the target sequence. In some embodiments, the transcription related protein is recruited to a region of the target gene in close proximity to the target sequence. In some embodiments, the transcription related protein is recruited to a region that is 100-200 bp, 200-300 bp, 300-400 bp, 400-500 bp, 500-600 bp, 600-700 bp, 700-800 bp, 800-900 bp, 900-1000 bp or more 3′ to the target sequence. In some embodiments, the effector domain comprises a protein that blocks or recruits one or more proteins that block access of a transcription factor to the target gene harboring the target sequence.

An effector domain alters a chemical modification state of DNA or histone residues associated with the DNA in a target gene. For example, an effector domain may deposit a chemical modification, or remove a chemical modification, such as DNA methylation, histone tail methylation, or histone tail acetylation at DNA nucleotides in or histone residues bound to a target gene. In some embodiments, an effector domain may directly or indirectly mediate or induce a chemical modification, or remove a chemical modification, such as DNA methylation, histone tail methylation, or histone tail acetylation at DNA nucleotides in or histone residues bound to a target gene. For example, an effector domain may place, deposit, or remove an initial epigenetic modification, e.g., DNA methylation, at one or more nucleotides in a target sequence of the target gene, and the epigenetic modification state may then spread to nucleotides 100, 200, 300, 400, 500, 600, 700, 800, 900, 1000, 1500, 2000, 2500, 3000 or more base pairs upstream or downstream of the initial epigenetic modification sites. The chemical modification deposited at target gene DNA nucleotides or histone residues may be in close proximity to a target sequence (sequence recognized by a DNA binding portion of an epigenetic editor) in the target gene, or may be distant from the target sequence. In some embodiments, an effector domain alters a chemical modification state of a nucleotide or histone tail bound to a nucleotide within 10, 20, 30, 40, 50, 60, 70, 80, 90, 100, 200, 300, 400, 500, 600, 700, 800, 900, or 1000 nucleotides flanking the target sequence. As used herein, “flanking” refers to nucleotide positions 5′ to the 5′ end of and 3′ to the 3′ end of a particular sequence, e.g. a target sequence. In some embodiments, an effector domain mediates or induces a chemical modification change of a nucleotide or a histone tail bound to a nucleotide distant from a target sequence. Without wishing to be bound by any theory, an epigenetic editor effector domain may initiate a chemical modification, e.g, DNA methylation, in one or more nucleotides of the target gene. Such modification may be initiated near the target sequence, and may subsequently spread to one or more nucleotides in the target gene distant from the target sequence. In some instances, additional proteins or transcription factors, for example, transcription repressors, methyltransferases, or transcription regulation scaffold proteins, are involved in the spreading of the chemical modification. In some embodiments, an effector domain initiates alteration of a chemical modification state of one or more nucleotides or one or more histone residues bound to one or more nucleotides within 10, 20, 30, 40, 50, 60, 70, 80, 90, 100, 200, 300, 400, 500 nucleotides flanking the target sequence, and the chemical modification state alteration spreads to one or more nucleotides at least 500, 600, 700, 800, 900, 1000, 1100, 1200, 1300, 1400, 1500, 1600, 1700, 1800, 1900, 2000 or more nucleotides from the target sequence in the target gene, either upstream or downstream of the target sequence. In certain embodiments, the chemical modification, e.g., methylation or demethylation, maybe initiated at less than 2, 3, 5, 10, 20, 30, 40, 50, or 100 nucleotides in the target gene and spreads to at least 100, 200, 300, 400, 500, 600, 700, 800, 900, 1000, 2000, or more nucleotides in the target gene. In some embodiments, the chemical modification spreads to nucleotides in the entire target gene. In some embodiments, the alteration in modification state is a DNA methylation state. In some embodiments, the alteration in modification state is a histone methylation state. In some embodiments, the alteration in modification state is a histone acetylation state.

In some embodiments, an effector domain makes an epigenetic modification at a target gene that increases or activates expression of the target gene. In some embodiments, an effector domain alters a chemical modification state of DNA or histone residues associated with the DNA in a target gene harboring the target sequence, thereby increasing expression of the target gene. In some embodiments, the alteration in chemical modification state comprises removal of a methyl group form a DNA nucleotide in the target gene. In some embodiments, the alteration in chemical modification state comprises acetylation of a histone tail bound to a DNA nucleotide in the target gene. In some embodiments, the alteration in chemical modification state comprises methylation of a histone tail bound to a DNA nucleotide in the target gene, e.g., a H3K4me1 methylation. In some embodiments, the alteration in chemical modification state comprises removal of an acetyl group from histone tail bound to a DNA nucleotide in the target gene, e.g., a H3K9me2 methylation. An epigenetic editor may initiate a chemical modification, in one or more nucleotides of the target gene, near the target sequence, which may subsequently spread to one or more nucleotides in the target gene distant from the target sequence, thereby increasing or activating expression of the target gene. In some instances, distant modification is solely mediated by the epigenetic editor. In some instances, additional proteins or transcription factors, for example, transcription activators, are involved in the spreading of the chemical modification. In some embodiments, an effector domain alters a chemical modification state of a nucleotide 50, 60, 70, 80, 90, 100, 200, 300, 400, 500, 600, 700, 800, 900, or1000 nucleotides flanking a target sequence in a target gene, thereby increasing expression of the target gene. In some embodiments, an effector domain initiates alteration of a chemical modification state of one or more nucleotides or one or more histone residues bound to one or more nucleotides within 10, 20, 30, 40, 50, 60, 70, 80, 90, 100, 200, 300, 400, 500 nucleotides flanking the target sequence, and the chemical modification state alteration spreads to one or more nucleotides at least 500, 600, 700, 800, 900, 1000, 1100, 1200, 1300, 1400, 1500, 1600, 1700, 1800, 1900, 2000 or more nucleotides flanking the target sequence in the target gene, thereby increasing or activating expression of the target gene.

In some embodiments, an effector domain alters a chemical modification state, e.g., demethylation of a nucleotide, 100-200 nucleotides 5′ to the target sequence in the target gene, thereby increasing expression of the target gene. In some embodiments, an effector domain alters a chemical modification state of a nucleotide 200-300 nucleotides 5′ to the target sequence in the target gene, thereby increasing expression of the target gene. In some embodiments, an effector domain alters a chemical modification state of a nucleotide 300-400 nucleotides 5′ to the target sequence in the target gene, thereby increasing expression of the target gene. In some embodiments, an effector domain alters a chemical modification state of a nucleotide 400-500 nucleotides 5′ to the target sequence in the target gene, thereby increasing expression of the target gene. In some embodiments, an effector domain alters a chemical modification state of a nucleotide 500-600 nucleotides 5′ to the target sequence in the target gene, thereby increasing expression of the target gene. In some embodiments, an effector domain alters a chemical modification state of a nucleotide 600-700 nucleotides 5′ to the target sequence in the target gene, thereby increasing expression of the target gene. In some embodiments, an effector domain alters a chemical modification state of a nucleotide 700-800 nucleotides 5′ to the target sequence in the target gene, thereby increasing expression of the target gene. In some embodiments, an effector domain initiates alteration of a chemical modification state of one or more nucleotides or one or more histone residues bound to one or more nucleotides within 10, 20, 30, 40, 50, 60, 70, 80, 90, 100, 200, 300, 400, 500 nucleotides flanking the target sequence, and the chemical modification state alteration spreads to one or more nucleotides at least 500, 600, 700, 800, 900, 1000, 1100, 1200, 1300, 1400, 1500, 1600, 1700, 1800, 1900, 2000 or more nucleotides 5′ to the target sequence in the target gene, thereby increasing or activating expression of the target gene, thereby increasing expression of the target gene.

In some embodiments, an effector domain alters a chemical modification state, e.g., demethylation of a nucleotide, of a nucleotide 100-200 nucleotides 3′ to the target sequence in the target gene, thereby increasing expression of the target gene. In some embodiments, an effector domain alters a chemical modification state of a nucleotide 200-300 nucleotides 3′ to the target sequence in the target gene, thereby increasing expression of the target gene. In some embodiments, an effector domain alters a chemical modification state of a nucleotide 300-400 nucleotides 3′ to the target sequence in the target gene, thereby increasing expression of the target gene. In some embodiments, an effector domain alters a chemical modification state of a nucleotide 400-500 nucleotides 3′ to the target sequence in the target gene, thereby increasing expression of the target gene. In some embodiments, an effector domain alters a chemical modification state of a nucleotide 500-600 nucleotides 3′ to the target sequence in the target gene, thereby increasing expression of the target gene. In some embodiments, an effector domain alters a chemical modification state of a nucleotide 600-700 nucleotides 3′ to the target sequence in the target gene, thereby increasing expression of the target gene. In some embodiments, an effector domain alters a chemical modification state of a nucleotide 700-800 nucleotides 3′ to the target sequence in the target gene, thereby increasing expression of the target gene. In some embodiments, the chemical modification state is a methylation state. In some embodiments, the effector domain of an epigenetic effector results in demethylation of one or more nucleotides in the target gene, thereby increasing expression of the target gene. In some embodiments, an effector domain initiates alteration of a chemical modification state, e.g. DNA demethylation, of one or more nucleotides or one or more histone residues bound to one or more nucleotides within 10, 20, 30, 40, 50, 60, 70, 80, 90, 100, 200, 300, 400, 500 nucleotides flanking the target sequence, and the chemical modification state alteration spreads to one or more nucleotides at least 500, 600, 700, 800, 900, 1000, 1100, 1200, 1300, 1400, 1500, 1600, 1700, 1800, 1900, 2000 or more nucleotides 3′ to the target sequence in the target gene, thereby increasing or activating expression of the target gene, thereby increasing expression of the target gene.

In some embodiments, an effector domain alters a histone modification state of a histone associated with or bound to the target gene. For example, an effector domain may deposit a modification on one or more lysine residues of histone tails of histones associated with the target gene. The histone amino acid residues modified may be within the vicinity of the target sequence within the target gene. In some embodiments, an effector domain alters a histone modification state 50, 60, 70, 80, 90, 100, 200, 300, 400, 500, 600, 700, 800, 900, 1000 or more nucleotides 5′ or 3′ to the target sequence in the target gene, thereby increasing expression of the target gene. In some embodiments, an effector domain alters a histone modification state 100-200 nucleotides 5′ to the target sequence in the target gene, thereby increasing expression of the target gene. In some embodiments, an effector domain alters a histone modification state 200-300 nucleotides 5′ to the target sequence in the target gene, thereby increasing expression of the target gene. In some embodiments, an effector domain alters a histone modification state 300-400 nucleotides 5′ to the target sequence in the target gene, thereby increasing expression of the target gene. In some embodiments, an effector domain alters a histone modification state 400-500 nucleotides 5′ to the target sequence in the target gene, thereby increasing expression of the target gene. In some embodiments, an effector domain alters a histone modification state 500-600 nucleotides 5′ to the target sequence in the target gene, thereby increasing expression of the target gene. In some embodiments, an effector domain alters a histone modification state 600-700 nucleotides 5′ to the target sequence in the target gene, thereby increasing expression of the target gene. In some embodiments, an effector domain alters a histone modification state 700-800 nucleotides 5′ to the target sequence in the target gene, thereby increasing expression of the target gene. In some embodiments, an effector domain alters a histone modification state 50, 60, 70, 80, 90, 100, 200, 300, 400, 500, 600, 700, 800, 900, 1000 or more nucleotides 5′ or 3′ to the target sequence in the target gene, thereby increasing expression of the target gene. In some embodiments, an effector domain alters a histone modification state 100-200 nucleotides 3′ to the target sequence in the target gene, thereby increasing expression of the target gene. In some embodiments, an effector domain alters a histone modification state 200-300 nucleotides 3′ to the target sequence in the target gene, thereby increasing expression of the target gene. In some embodiments, an effector domain alters a histone modification state 300-400 nucleotides 3′ to the target sequence in the target gene, thereby increasing expression of the target gene. In some embodiments, an effector domain alters a histone modification state 400-500 nucleotides 3′ to the target sequence in the target gene, thereby increasing expression of the target gene. In some embodiments, an effector domain alters a histone modification state 500-600 nucleotides 3′ to the target sequence in the target gene, thereby increasing expression of the target gene. In some embodiments, an effector domain alters a histone modification state 600-700 nucleotides 3′ to the target sequence in the target gene, thereby increasing expression of the target gene. In some embodiments, an effector domain alters a histone modification state 700-800 nucleotides 3′ to the target sequence in the target gene, thereby increasing expression of the target gene. In some embodiments, the histone modification state is a acetylation state. In some embodiments, the effector domain of an epigenetic effector results in acetylation of one or more histone tails of histones associated with the target gene, thereby increasing expression of the target gene. In some embodiments, the histone modification state is a methylation state. In some embodiments, the epigenetic effector results in H3K4 or H3K79 methylation (e.g. one or more of a H3K4me2, H3K4me3, and H3K79me3 methylation) at one or more histone tails associated with the target gene, thereby increasing expression of the target gene.

In some embodiments, an effector domain makes an epigenetic modification at a target gene that represses, decreases, or silences expression of the target gene. In some embodiments, an effector domain alters a chemical modification state of DNA or histone residues associated with the DNA in a target gene harboring the target sequence, thereby reducing or silencing expression of the target gene. Epigenetic editors that decrease expression of a target gene may comprise multiple effector domains, resulting in multiple modifications to a target gene, for example, both DNA methylation and histone tail de-acetylation. In some embodiments, an effector domain alters a chemical modification state of DNA in the target gene or histone bound to the target gene near the target sequence, thereby decreasing expression of the target gene. In some embodiments, an effector domain alters a chemical modification state of DNA in the target gene or histone bound to the target gene distant from the target sequence in the target gene, thereby decreasing expression of the target gene. In some embodiments, an effector domain mediates or induces a chemical modification state of DNA in the target gene or histone bound to the target gene that are distant from the target sequence in the target gene. For example, an epigenetic editor may initiate a chemical modification, e.g, DNA methylation, in one or more nucleotides of the target gene. Such modification may be initiated near the target sequence, and may subsequently spread to one or more nucleotides in the target gene distant from the target sequence, thereby decreasing expression of the target gene. In some instances, the distant modification is solely mediated by the epigenetic editor. In some instances, additional proteins or transcription factors, for example, transcription repressors, methyltransferases, or transcription regulation scaffold proteins, are involved in the spreading of the chemical modification. In some embodiments, an effector domain alters a chemical modification state of a nucleotide 50, 60, 70, 80, 90, 100, 200, 300, 400, 500, 600, 700, 800, 900, 1000 1100, 1200, 1300, 1400, 1500, 1600, 1700, 1800, 1900, 2000 or more nucleotides 5′ or 3′ to the target sequence in the target gene, thereby reducing or silencing expression of the target gene. In some embodiments, an effector domain alters a chemical modification state, e.g., DNA methylation, of one or more nucleotides in close proximity to the target gene, and the altered chemical modification state subsequently spreads to nucleotides 100, 200, 300, 400, 500, 600, 700, 800, 900, 1000, 1100, 1200, 1300, 1400, 1500, 1600, 1700, 1800, 1900, 2000 or more nucleotides 5′ or 3′ to the target sequence in the target gene, thereby reducing or silencing expression of the target gene.

In some embodiments, an effector domain alters a chemical modification state, e.g., DNA methylation, of one or more nucleotides or one or more histone residues bound to one or more nucleotides within 10, 20, 30, 40, 50, 60, 70, 80, 90, 100, 200, 300, 400, 500 nucleotides flanking the target sequence, and the altered chemical modification state subsequently spreads to nucleotides 100, 200, 300, 400, 500, 600, 700, 800, 900, 1000, 1100, 1200, 1300, 1400, 1500, 1600, 1700, 1800, 1900, 2000 or more nucleotides 5′ or 3′ to the target sequence in the target gene, thereby reducing or silencing expression of the target gene.

In some embodiments, an effector domain alters a chemical modification state of a nucleotide 100-200 nucleotides 5′ to the target sequence in the target gene, thereby reducing or silencing expression of the target gene. In some embodiments, an effector domain alters a chemical modification state of a nucleotide 200-300 nucleotides 5′ to the target sequence in the target gene, thereby reducing or silencing expression of the target gene. In some embodiments, an effector domain alters a chemical modification state of a nucleotide 300-400 nucleotides 5′ to the target sequence in the target gene, thereby reducing or silencing expression of the target gene. In some embodiments, an effector domain alters a chemical modification state of a nucleotide 400-500 nucleotides 5′ to the target sequence in the target gene, thereby reducing or silencing expression of the target gene. In some embodiments, an effector domain alters a chemical modification state of a nucleotide 500-600 nucleotides 5′ to the target sequence in the target gene, thereby reducing or silencing expression of the target gene. In some embodiments, an effector domain alters a chemical modification state of a nucleotide 600-700 nucleotides 5′ to the target sequence in the target gene, thereby reducing or silencing expression of the target gene. In some embodiments, an effector domain alters a chemical modification state of a nucleotide 700-800 nucleotides 5′ to the target sequence in the target gene, thereby reducing or silencing expression of the target gene.

In some embodiments, an effector domain alters a chemical modification state of a nucleotide 50, 60, 70, 80, 90, 100, 200, 300, 400, 500, 600, 700, 800, 900, 1000 or more nucleotides 5′ or 3′ to the target sequence in the target gene, thereby reducing or silencing expression of the target gene. In some embodiments, an effector domain initiates alteration of a chemical modification state, e.g. DNA methylation, of one or more nucleotides or one or more histone residues bound to one or more nucleotides within 10, 20, 30, 40, 50, 60, 70, 80, 90, 100, 200, 300, 400, 500 nucleotides flanking the target sequence, and the chemical modification state alteration spreads to one or more nucleotides at least 500, 600, 700, 800, 900, 1000, 1100, 1200, 1300, 1400, 1500, 1600, 1700, 1800, 1900, 2000 or more nucleotides 3′ to the target sequence in the target gene, thereby increasing or activating expression of the target gene, thereby increasing expression of the target gene.

In some embodiments, an effector domain alters a chemical modification state of a nucleotide 100-200 nucleotides 3′ to the target sequence in the target gene, thereby reducing or silencing expression of the target gene. In some embodiments, an effector domain alters a chemical modification state of a nucleotide 200-300 nucleotides 3′ to the target sequence in the target gene, thereby reducing or silencing expression of the target gene. In some embodiments, an effector domain alters a chemical modification state of a nucleotide 300-400 nucleotides 3′ to the target sequence in the target gene, thereby reducing or silencing expression of the target gene. In some embodiments, an effector domain alters a chemical modification state of a nucleotide 400-500 nucleotides 3′ to the target sequence in the target gene, thereby reducing or silencing expression of the target gene. In some embodiments, an effector domain alters a chemical modification state of a nucleotide 500-600 nucleotides 3′ to the target sequence in the target gene, thereby reducing or silencing expression of the target gene. In some embodiments, an effector domain alters a chemical modification state of a nucleotide 600-700 nucleotides 3′ to the target sequence in the target gene, thereby reducing or silencing expression of the target gene. In some embodiments, an effector domain alters a chemical modification state of a nucleotide 700-800 nucleotides 3′ to the target sequence in the target gene, thereby reducing or silencing expression of the target gene. In some embodiments, the chemical modification state is a methylation state. In some embodiments, the effector domain of an epigenetic effector results in methylation of one or more nucleotides in the target gene, thereby reducing or silencing expression of the target gene. In some embodiments, an effector domain initiates alteration of a chemical modification state, e.g. DNA methylation, of one or more nucleotides or one or more histone residues bound to one or more nucleotides within 10, 20, 30, 40, 50, 60, 70, 80, 90, 100, 200, 300, 400, 500 nucleotides flanking the target sequence, and the chemical modification state alteration spreads to one or more nucleotides at least 500, 600, 700, 800, 900, 1000, 1100, 1200, 1300, 1400, 1500, 1600, 1700, 1800, 1900, 2000 or more nucleotides 5′ to the target sequence in the target gene, thereby increasing or activating expression of the target gene, thereby increasing expression of the target gene.

In some embodiments, an effector domain alters a histone modification state of a histone associated with or bound to the target gene. For example, an effector domain may deposit a modification on one or more lysine residues of histone tails of histones associated with the target gene. The histone amino acid residues modified may be within the vicinity of the target sequence within the target gene. In some embodiments, an effector domain alters a histone modification state 50, 60, 70, 80, 90, 100, 200, 300, 400, 500, 600, 700, 800, 900, 1000 or more nucleotides 5′ or 3′ to the target sequence in the target gene, thereby reducing or silencing expression of the target gene. In some embodiments, an effector domain alters a histone modification state 100-200 nucleotides 5′ to the target sequence in the target gene, thereby reducing or silencing expression of the target gene. In some embodiments, an effector domain alters a histone modification state 200-300 nucleotides 5′ to the target sequence in the target gene, thereby reducing or silencing expression of the target gene. In some embodiments, an effector domain alters a histone modification state 300-400 nucleotides 5′ to the target sequence in the target gene, thereby reducing or silencing expression of the target gene. In some embodiments, an effector domain alters a histone modification state 400-500 nucleotides 5′ to the target sequence in the target gene, thereby reducing or silencing expression of the target gene. In some embodiments, an effector domain alters a histone modification state 500-600 nucleotides 5′ to the target sequence in the target gene, thereby reducing or silencing expression of the target gene. In some embodiments, an effector domain alters a histone modification state 600-700 nucleotides 5′ to the target sequence in the target gene, thereby reducing or silencing expression of the target gene. In some embodiments, an effector domain alters a histone modification state 700-800 nucleotides 5′ to the target sequence in the target gene, thereby reducing or silencing expression of the target gene. In some embodiments, an effector domain alters a histone modification state 50, 60, 70, 80, 90, 100, 200, 300, 400, 500, 600, 700, 800, 900, 1000 or more nucleotides 5′ or 3′ to the target sequence in the target gene, thereby reducing or silencing expression of the target gene. In some embodiments, an effector domain alters a histone modification state 100-200 nucleotides 3′ to the target sequence in the target gene, thereby reducing or silencing expression of the target gene. In some embodiments, an effector domain alters a histone modification state 200-300 nucleotides 3′ to the target sequence in the target gene, thereby reducing or silencing expression of the target gene. In some embodiments, an effector domain alters a histone modification state 300-400 nucleotides 3′ to the target sequence in the target gene, thereby reducing or silencing expression of the target gene. In some embodiments, an effector domain alters a histone modification state 400-500 nucleotides 3′ to the target sequence in the target gene, thereby reducing or silencing expression of the target gene. In some embodiments, an effector domain alters a histone modification state 500-600 nucleotides 3′ to the target sequence in the target gene, thereby reducing or silencing expression of the target gene. In some embodiments, an effector domain alters a histone modification state 600-700 nucleotides 3′ to the target sequence in the target gene, thereby reducing or silencing expression of the target gene. In some embodiments, an effector domain alters a histone modification state 700-800 nucleotides 3′ to the target sequence in the target gene, thereby reducing or silencing expression of the target gene. In some embodiments, the histone modification state is a acetylation state. In some embodiments, the effector domain of an epigenetic effector results in de-acetylation of one or more histone tails of histones associated with the target gene, thereby reducing or silencing expression of the target gene. In some embodiments, the histone modification state is a methylation state. In some embodiments, the epigenetic effector results in a H3K9, H3K27 or H4K20 methylation (e.g. one or more of a H3K9me2, H3K9me3, H3K27me2, H3K27me3, and H4K20me3 methylation) at one or more histone tails associated with the target gene, thereby reducing or silencing expression of the target gene.

In an aspect, also provided herein is an epigenetically edited chromosome or an epigenetically edited genome or cell comprising the epigenetically edited chromosome, wherein one or more target nucleotides in the epigenetically edited chromosome comprises an epigenetic modification mediated or induced by an epigenetic editor provided herein. For example, an epigenetically edited chromosome may comprise one or more methylated nucleotides as compared to a chromosome not contacted with an epigenetic editor. In some embodiments, the epigenetically edited chromosome comprises methylated CpGs. An epigenetically edited chromosome may comprise one or more types of epigenetic modifications as compared to an un-edited control chromosome of the same species, for example, epigenetic modifications to DNA nucleotides or histone tails of the chromosome. In some embodiments, an epigenetically edited chromosome comprises one or more methylated nucleotides as compared to a control chromosome not contacted with the epigenetic editor. In some embodiments, an epigenetically edited chromosome comprises one or more demethylated nucleotides as compared to a control chromosome not contacted with the epigenetic editor. In some embodiments, an epigenetically edited chromosome comprises one or more methylated histone tails as compared to a control chromosome not contacted with the epigenetic editor. In some embodiments, an epigenetically edited chromosome comprises one or more demethylated histone tails as compared to a control chromosome not contacted with the epigenetic editor. In some embodiments, an epigenetically edited chromosome comprises one or more acetylated histone tails as compared to a control chromosome not contacted with the epigenetic editor. In some embodiments, an epigenetically edited chromosome comprises one or more deacetylated histone tails as compared to a control chromosome not contacted with the epigenetic editor. In some embodiments, an epigenetically edited chromosome comprises one or more or any combination of epigenetic modifications, e.g, DNA methylation and histone deacetylation, DNA methylation and histone H3K9 methylation, DNA methylation and histone H3K4 demethylation, DNA demethylation and histone acetylation, DNA demethylation and histone H3K9 demethylation, DNA demethylation and histone H3K4 methylation, in any of the chromosome regions, e.g., chromosome regions as described herein, or any combination thereof. As used herein, a control chromosome may refer to the original epigenetic state, or unedited state, where a chromosome has not been contacted with an epigenetic editor as described herein. In some embodiments, a control chromosome may already bear epigenetic marks, e.g. DNA methylation, without being contacted with an epigenetic editor.

In some embodiments, all CpG dinucleotides within 2000 bps flanking a transcription start site of a gene in the epigenetically edited chromosome in a cell are methylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700 or more CpG dinucleotides within 2000 bps flanking a transcription start site of a gene in the epigenetically edited chromosome in a cell are methylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 2000 bps flanking a transcription start site of a gene in the epigenetically edited chromosome in a cell are methylated as compared to the original state of the gene or the gene in a comparable cell not contacted with the epigenetic editor. In some embodiments, one single CpG dinucleotide within 2000 bps flanking a transcription start site of a gene in the epigenetically edited chromosome in a cell is methylated as compared to the original state of the gene or the gene in a comparable cell not contacted with the epigenetic editor.

In some embodiments, all CpG dinucleotides within 2000 bps flanking a transcription start site of a gene in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700 or more CpG dinucleotides within 2000 bps flanking a transcription start site of a gene in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 2000 bps flanking a transcription start site of a gene in the epigenetically edited chromosome in a cell are demethylated as compared to the original state of the gene or the gene in a comparable cell not contacted with the epigenetic editor. In some embodiments, one single CpG dinucleotide within 2000 bps flanking a transcription start site of a gene in the epigenetically edited chromosome in a cell is demethylated as compared to the original state of the gene or the gene in a comparable cell not contacted with the epigenetic editor.

In some embodiments, all histone tails of histones bound to DNA nucleotides within 2000 bps flanking a transcription start site of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of histones bound to DNAs within 2000 bps flanking a transcription start site of a gene in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 2000 bps flanking a transcription start site of a gene in the epigenetically edited chromosome in a cell are methylated as compared to the original state of the chromosome or the chromosome in a comparable cell not contacted with the epigenetic editor. In some embodiments, one single histone tail of histones bound to DNAs within 2000 bps flanking a transcription start site of a gene in the epigenetically edited chromosome in a cell is methylated as compared to the original state of the chromosome or the chromosome in a comparable cell not contacted with the epigenetic editor. In some embodiments, one single histone octamer bound to DNAs within 2000 bps flanking a transcription start site of a gene in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 2000 bps flanking a transcription start site of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of histones bound to DNAs within 2000 bps flanking a transcription start site of a gene in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 2000 bps flanking a transcription start site of a gene in the epigenetically edited chromosome in a cell are demethylated as compared to the original state of the chromosome or the chromosome in a comparable cell not contacted with the epigenetic editor. In some embodiments, one single histone tail of histones bound to DNAs within 2000 bps flanking a transcription start site of a gene in the epigenetically edited chromosome in a cell is demethylated as compared to the original state of the chromosome or the chromosome in a comparable cell not contacted with the epigenetic editor. In some embodiments, one single histone octamer bound to DNAs within 2000 bps flanking a transcription start site of a gene in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 2000 bps flanking a transcription start site of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of histones bound to DNAs within 2000 bps flanking a transcription start site of a gene in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 2000 bps flanking a transcription start site of a gene in the epigenetically edited chromosome in a cell are acetylated as compared to the original state of the chromosome or the chromosome in a comparable cell not contacted with the epigenetic editor. In some embodiments, one single histone tail of histones bound to DNAs within 2000 bps flanking a transcription start site of a gene in the epigenetically edited chromosome in a cell is acetylated as compared to the original state of the chromosome or the chromosome in a comparable cell not contacted with the epigenetic editor. In some embodiments, one single histone octamer bound to DNAs within 2000 bps flanking a transcription start site of a gene in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 2000 bps flanking a transcription start site of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of histones bound to DNAs within 2000 bps flanking a transcription start site of a gene in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 2000 bps flanking a transcription start site of a gene in the epigenetically edited chromosome in a cell are deacetylated as compared to the original state of the chromosome or the chromosome in a comparable cell not contacted with the epigenetic editor. In some embodiments, one single histone tail of histones bound to DNAs within 2000 bps flanking a transcription start site of a gene in the epigenetically edited chromosome in a cell is deacetylated as compared to the original state of the chromosome or the chromosome in a comparable cell not contacted with the epigenetic editor. In some embodiments, one single histone octamer bound to DNAs within 2000 bps flanking a transcription start site of a gene in the epigenetically edited chromosome in a cell is deacetylated as compared to the original state of the chromosome or the chromosome in a comparable cell not contacted with the epigenetic editor. In some embodiments, all CpG dinucleotides within 1500 bp flanking a transcription start site of a gene in the epigenetically edited chromosome in a cell are methylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 550, 500, 600, 650, 700 or more CpG dinucleotides within 1500 bps flanking a transcription start site of a gene in the epigenetically edited chromosome in a cell are methylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 1500 bps flanking a transcription start site of a gene in the epigenetically edited chromosome in a cell are methylated as compared to the original state of the gene or the gene in a comparable cell not contacted with the epigenetic editor. In some embodiments, one single CpG dinucleotide within 1500 bps flanking a transcription start site of a gene in the epigenetically edited chromosome in a cell is methylated as compared to the original state of the gene or the gene in a comparable cell not contacted with the epigenetic editor.

In some embodiments, all CpG dinucleotides within 1500 bps flanking a transcription start site of a gene in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700 or more CpG dinucleotides within 1500 bps flanking a transcription start site of a gene in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 1500 bps flanking a transcription start site of a gene in the epigenetically edited chromosome in a cell are demethylated as compared to the original state of the gene or the gene in a comparable cell not contacted with the epigenetic editor. In some embodiments, one single CpG dinucleotide within 1500 bps flanking a transcription start site of a gene in the epigenetically edited chromosome in a cell is demethylated as compared to the original state of the gene or the gene in a comparable cell not contacted with the epigenetic editor.

In some embodiments, all histone tails of histones bound to DNA nucleotides within 1500 bps flanking a transcription start site of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more histone tails of histones bound to DNAs within 1500 bps flanking a transcription start site of a gene in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1500 bps flanking a transcription start site of a gene in the epigenetically edited chromosome in a cell are methylated as compared to the original state of the chromosome or the chromosome in a comparable cell not contacted with the epigenetic editor. In some embodiments, one single histone tail of histones bound to DNAs within 1500 bps flanking a transcription start site of a gene in the epigenetically edited chromosome in a cell is methylated as compared to the original state of the chromosome or the chromosome in a comparable cell not contacted with the epigenetic editor. In some embodiments, one single histone octamer bound to DNAs within 1500 bps flanking a transcription start site of a gene in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 1500 bps flanking a transcription start site of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more histone tails of histones bound to DNAs within 1500 bps flanking a transcription start site of a gene in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1500 bps flanking a transcription start site of a gene in the epigenetically edited chromosome in a cell are demethylated as compared to the original state of the chromosome or the chromosome in a comparable cell not contacted with the epigenetic editor. In some embodiments, one single histone tail of histones bound to DNAs within 1500 bps flanking a transcription start site of a gene in the epigenetically edited chromosome in a cell is demethylated as compared to the original state of the chromosome or the chromosome in a comparable cell not contacted with the epigenetic editor. In some embodiments, one single histone octamer bound to DNAs within 1500 bps flanking a transcription start site of a gene in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 1500 bps flanking a transcription start site of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more histone tails of histones bound to DNAs within 1500 bps flanking a transcription start site of a gene in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1500 bps flanking a transcription start site of a gene in the epigenetically edited chromosome in a cell are acetylated as compared to the original state of the chromosome or the chromosome in a comparable cell not contacted with the epigenetic editor. In some embodiments, one single histone tail of histones bound to DNAs within 1500 bps flanking a transcription start site of a gene in the epigenetically edited chromosome in a cell is acetylated as compared to the original state of the chromosome or the chromosome in a comparable cell not contacted with the epigenetic editor. In some embodiments, one single histone octamer bound to DNAs within 1500 bps flanking a transcription start site of a gene in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 1500 bps flanking a transcription start site of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more histone tails of histones bound to DNAs within 1500 bps flanking a transcription start site of a gene in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1500 bps flanking a transcription start site of a gene in the epigenetically edited chromosome in a cell are deacetylated as compared to the original state of the chromosome or the chromosome in a comparable cell not contacted with the epigenetic editor. In some embodiments, one single histone tail of histones bound to DNAs within 1500 bps flanking a transcription start site of a gene in the epigenetically edited chromosome in a cell is deacetylated as compared to the original state of the chromosome or the chromosome in a comparable cell not contacted with the epigenetic editor. In some embodiments, one single histone octamer bound to DNAs within 1500 bps flanking a transcription start site of a gene in the epigenetically edited chromosome in a cell is deacetylated as compared to the original state of the chromosome or the chromosome in a comparable cell not contacted with the epigenetic editor. In some embodiments, all CpG dinucleotides within 1000 bps flanking a transcription start site of a gene in the epigenetically edited chromosome in a cell are methylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500 or more CpG dinucleotides within 1000 bps flanking a transcription start site of a gene in the epigenetically edited chromosome in a cell are methylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 1000 bps flanking a transcription start site of a gene in the epigenetically edited chromosome in a cell are methylated as compared to the original state of the gene or the gene in a comparable cell not contacted with the epigenetic editor. In some embodiments, one single CpG dinucleotide within 1000 bps flanking a transcription start site of a gene in the epigenetically edited chromosome in a cell is methylated as compared to the original state of the gene or the gene in a comparable cell not contacted with the epigenetic editor.

In some embodiments, all CpG dinucleotides within 1000 bps flanking a transcription start site of a gene in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500 or more CpG dinucleotides within 1000 bps flanking a transcription start site of a gene in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 1000 bps flanking a transcription start site of a gene in the epigenetically edited chromosome in a cell are demethylated as compared to the original state of the gene or the gene in a comparable cell not contacted with the epigenetic editor. In some embodiments, one single CpG dinucleotide within 1000 bps flanking a transcription start site of a gene in the epigenetically edited chromosome in a cell is demethylated as compared to the original state of the gene or the gene in a comparable cell not contacted with the epigenetic editor.

In some embodiments, all histone tails of histones bound to DNA nucleotides within 1000 bps flanking a transcription start site of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60 or more histone tails of histones bound to DNAs within 1000 bps flanking a transcription start site of a gene in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1000 bps flanking a transcription start site of a gene in the epigenetically edited chromosome in a cell are methylated as compared to the original state of the chromosome or the chromosome in a comparable cell not contacted with the epigenetic editor. In some embodiments, one single histone tail of histones bound to DNAs within 1000 bps flanking a transcription start site of a gene in the epigenetically edited chromosome in a cell is methylated as compared to the original state of the chromosome or the chromosome in a comparable cell not contacted with the epigenetic editor. In some embodiments, one single histone octamer bound to DNAs within 1000 bps flanking a transcription start site of a gene in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 1000 bps flanking a transcription start site of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60 or more histone tails of histones bound to DNAs within 1000 bps flanking a transcription start site of a gene in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1000 bps flanking a transcription start site of a gene in the epigenetically edited chromosome in a cell are demethylated as compared to the original state of the chromosome or the chromosome in a comparable cell not contacted with the epigenetic editor. In some embodiments, one single histone tail of histones bound to DNAs within 1000 bps flanking a transcription start site of a gene in the epigenetically edited chromosome in a cell is demethylated as compared to the original state of the chromosome or the chromosome in a comparable cell not contacted with the epigenetic editor. In some embodiments, one single histone octamer bound to DNAs within 1000 bps flanking a transcription start site of a gene in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 1000 bps flanking a transcription start site of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60 or more histone tails of histones bound to DNAs within 1000 bps flanking a transcription start site of a gene in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1000 bps flanking a transcription start site of a gene in the epigenetically edited chromosome in a cell are acetylated as compared to the original state of the chromosome or the chromosome in a comparable cell not contacted with the epigenetic editor. In some embodiments, one single histone tail of histones bound to DNAs within 1000 bps flanking a transcription start site of a gene in the epigenetically edited chromosome in a cell is acetylated as compared to the original state of the chromosome or the chromosome in a comparable cell not contacted with the epigenetic editor. In some embodiments, one single histone octamer bound to DNAs within 1000 bps flanking a transcription start site of a gene in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 1000 bps flanking a transcription start site of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60 or more histone tails of histones bound to DNAs within 1000 bps flanking a transcription start site of a gene in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55% 60%, 65%, 70%, 75%, 80%, 85%, 90%, 95%, 96%, 97%, 98%, or 99% of histone tails of histones bound to DNAs within 1000 bps flanking a transcription start site of a gene in the epigenetically edited chromosome in a cell are deacetylated as compared to the original state of the chromosome or the chromosome in a comparable cell not contacted with the epigenetic editor. In some embodiments, one single histone tail of histones bound to DNAs within 1000 bps flanking a transcription start site of a gene in the epigenetically edited chromosome in a cell is deacetylated as compared to the original state of the chromosome or the chromosome in a comparable cell not contacted with the epigenetic editor. In some embodiments, one single histone octamer bound to DNAs within 1000 bps flanking a transcription start site of a gene in the epigenetically edited chromosome in a cell is deacetylated as compared to the original state of the chromosome or the chromosome in a comparable cell not contacted with the epigenetic editor. In some embodiments, all CpG dinucleotides within 500 bps flanking a transcription start site of a gene in the epigenetically edited chromosome in a cell are methylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200 or more CpG dinucleotides within 500 bps flanking a transcription start site of a gene in the epigenetically edited chromosome in a cell are methylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 500 bps flanking a transcription start site of a gene in the epigenetically edited chromosome in a cell are methylated as compared to the original state of the gene or the gene in a comparable cell not contacted with the epigenetic editor. In some embodiments, one single CpG dinucleotide within 500 bps flanking a transcription start site of a gene in the epigenetically edited chromosome in a cell is methylated as compared to the original state of the gene or the gene in a comparable cell not contacted with the epigenetic editor.

In some embodiments, all CpG dinucleotides within 500 bps flanking a transcription start site of a gene in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200 or more CpG dinucleotides within 500 bps flanking a transcription start site of a gene in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 500 bps flanking a transcription start site of a gene in the epigenetically edited chromosome in a cell are demethylated as compared to the original state of the gene or the gene in a comparable cell not contacted with the epigenetic editor. In some embodiments, one single CpG dinucleotide within 500 bps flanking a transcription start site of a gene in the epigenetically edited chromosome in a cell is demethylated as compared to the original state of the gene or the gene in a comparable cell not contacted with the epigenetic editor.

In some embodiments, all histone tails of histones bound to DNA nucleotides within 500 bps flanking a transcription start site of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30 or more histone tails of histones bound to DNAs within 500 bps flanking a transcription start site of a gene in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 500 bps flanking a transcription start site of a gene in the epigenetically edited chromosome in a cell are methylated as compared to the original state of the chromosome or the chromosome in a comparable cell not contacted with the epigenetic editor. In some embodiments, one single histone tail of histones bound to DNAs within 500 bps flanking a transcription start site of a gene in the epigenetically edited chromosome in a cell is methylated as compared to the original state of the chromosome or the chromosome in a comparable cell not contacted with the epigenetic editor. In some embodiments, one single histone octamer bound to DNAs within 500 bps flanking a transcription start site of a gene in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 500 bps flanking a transcription start site of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30 or more histone tails of histones bound to DNAs within 500 bps flanking a transcription start site of a gene in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 500 bps flanking a transcription start site of a gene in the epigenetically edited chromosome in a cell are demethylated as compared to the original state of the chromosome or the chromosome in a comparable cell not contacted with the epigenetic editor. In some embodiments, one single histone tail of histones bound to DNAs within 500 bps flanking a transcription start site of a gene in the epigenetically edited chromosome in a cell is demethylated as compared to the original state of the chromosome or the chromosome in a comparable cell not contacted with the epigenetic editor. In some embodiments, one single histone octamer bound to DNAs within 500 bps flanking a transcription start site of a gene in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 500 bps flanking a transcription start site of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30 or more histone tails of histones bound to DNAs within 500 bps flanking a transcription start site of a gene in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 500 bps flanking a transcription start site of a gene in the epigenetically edited chromosome in a cell are acetylated as compared to the original state of the chromosome or the chromosome in a comparable cell not contacted with the epigenetic editor. In some embodiments, one single histone tail of histones bound to DNAs within 500 bps flanking a transcription start site of a gene in the epigenetically edited chromosome in a cell is acetylated as compared to the original state of the chromosome or the chromosome in a comparable cell not contacted with the epigenetic editor. In some embodiments, one single histone octamer bound to DNAs within 500 bps flanking a transcription start site of a gene in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 500 bps flanking a transcription start site of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30 or more histone tails of histones bound to DNAs within 500 bps flanking a transcription start site of a gene in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 500 bps flanking a transcription start site of a gene in the epigenetically edited chromosome in a cell are deacetylated as compared to the original state of the chromosome or the chromosome in a comparable cell not contacted with the epigenetic editor. In some embodiments, one single histone tail of histones bound to DNAs within 500 bps flanking a transcription start site of a gene in the epigenetically edited chromosome in a cell is deacetylated as compared to the original state of the chromosome or the chromosome in a comparable cell not contacted with the epigenetic editor. In some embodiments, one single histone octamer bound to DNAs within 500 bps flanking a transcription start site of a gene in the epigenetically edited chromosome in a cell is deacetylated as compared to the original state of the chromosome or the chromosome in a comparable cell not contacted with the epigenetic editor.

In some embodiments, all CpG dinucleotides within 200 bps flanking a transcription start site of a gene in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90 or more CpG dinucleotides within 200 bps flanking a transcription start site of a gene in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 200 bps flanking a transcription start site of a gene in the epigenetically edited chromosome in a cell are demethylated as compared to the original state of the gene or the gene in a comparable cell not contacted with the epigenetic editor. In some embodiments, one single CpG dinucleotide within 200 bps flanking a transcription start site of a gene in the epigenetically edited chromosome in a cell is demethylated as compared to the original state of the gene or the gene in a comparable cell not contacted with the epigenetic editor.

In some embodiments, all histone tails of histones bound to DNA nucleotides within 200 bps flanking a transcription start site of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20 or more histone tails of histones bound to DNAs within 200 bps flanking a transcription start site of a gene in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 200 bps flanking a transcription start site of a gene in the epigenetically edited chromosome in a cell are methylated as compared to the original state of the chromosome or the chromosome in a comparable cell not contacted with the epigenetic editor. In some embodiments, one single histone tail of histones bound to DNAs within 200 bps flanking a transcription start site of a gene in the epigenetically edited chromosome in a cell is methylated as compared to the original state of the chromosome or the chromosome in a comparable cell not contacted with the epigenetic editor. In some embodiments, one single histone octamer bound to DNAs within 200 bps flanking a transcription start site of a gene in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 200 bps flanking a transcription start site of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20 or more histone tails of histones bound to DNAs within 200 bps flanking a transcription start site of a gene in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 200 bps flanking a transcription start site of a gene in the epigenetically edited chromosome in a cell are demethylated as compared to the original state of the chromosome or the chromosome in a comparable cell not contacted with the epigenetic editor. In some embodiments, one single histone tail of histones bound to DNAs within 200 bps flanking a transcription start site of a gene in the epigenetically edited chromosome in a cell is demethylated as compared to the original state of the chromosome or the chromosome in a comparable cell not contacted with the epigenetic editor. In some embodiments, one single histone octamer bound to DNAs within 200 bps flanking a transcription start site of a gene in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 200 bps flanking a transcription start site of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20 or more histone tails of histones bound to DNAs within 200 bps flanking a transcription start site of a gene in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 200 bps flanking a transcription start site of a gene in the epigenetically edited chromosome in a cell are acetylated as compared to the original state of the chromosome or the chromosome in a comparable cell not contacted with the epigenetic editor. In some embodiments, one single histone tail of histones bound to DNAs within 200 bps flanking a transcription start site of a gene in the epigenetically edited chromosome in a cell is acetylated as compared to the original state of the chromosome or the chromosome in a comparable cell not contacted with the epigenetic editor. In some embodiments, one single histone octamer bound to DNAs within 200 bps flanking a transcription start site of a gene in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 200 bps flanking a transcription start site of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20 or more histone tails of histones bound to DNAs within 200 bps flanking a transcription start site of a gene in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 200 bps flanking a transcription start site of a gene in the epigenetically edited chromosome in a cell are deacetylated as compared to the original state of the chromosome or the chromosome in a comparable cell not contacted with the epigenetic editor. In some embodiments, one single histone tail of histones bound to DNAs within 200 bps flanking a transcription start site of a gene in the epigenetically edited chromosome in a cell is deacetylated as compared to the original state of the chromosome or the chromosome in a comparable cell not contacted with the epigenetic editor. In some embodiments, one single histone octamer bound to DNAs within 200 bps flanking a transcription start site of a gene in the epigenetically edited chromosome in a cell is deacetylated as compared to the original state of the chromosome or the chromosome in a comparable cell not contacted with the epigenetic editor.

In some embodiments, all CpG dinucleotides within 2000 bps flanking a promoter sequence of a gene in the epigenetically edited chromosome in a cell are methylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700 or more CpG dinucleotides within 2000 bps flanking a promoter sequence of a gene in the epigenetically edited chromosome in a cell are methylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 2000 bps flanking a promoter sequence of a gene in the epigenetically edited chromosome in a cell are methylated as compared to the original state of the gene or the gene in a comparable cell not contacted with the epigenetic editor. In some embodiments, one single CpG dinucleotide within 2000 bps flanking a promoter sequence of a gene in the epigenetically edited chromosome in a cell is methylated as compared to the original state of the gene or the gene in a comparable cell not contacted with the epigenetic editor.

In some embodiments, all CpG dinucleotides within 2000 bps flanking a promoter sequence of a gene in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700 or more CpG dinucleotides within 2000 bps flanking a promoter sequence of a gene in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 2000 bps flanking a promoter sequence of a gene in the epigenetically edited chromosome in a cell are demethylated as compared to the original state of the gene or the gene in a comparable cell not contacted with the epigenetic editor. In some embodiments, one single CpG dinucleotide within 2000 bps flanking a promoter sequence of a gene in the epigenetically edited chromosome in a cell is demethylated as compared to the original state of the gene or the gene in a comparable cell not contacted with the epigenetic editor.

In some embodiments, all histone tails of histones bound to DNA nucleotides within 2000 bps flanking a promoter sequence of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of histones bound to DNAs within 2000 bps flanking a promoter sequence of a gene in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 2000 bps flanking a promoter sequence of a gene in the epigenetically edited chromosome in a cell are methylated as compared to the original state of the chromosome or the chromosome in a comparable cell not contacted with the epigenetic editor. In some embodiments, one single histone tail of histones bound to DNAs within 2000 bps flanking a promoter sequence of a gene in the epigenetically edited chromosome in a cell is methylated as compared to the original state of the chromosome or the chromosome in a comparable cell not contacted with the epigenetic editor. In some embodiments, one single histone octamer bound to DNAs within 2000 bps flanking a promoter sequence of a gene in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 2000 bps flanking a promoter sequence of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of histones bound to DNAs within 2000 bps flanking a promoter sequence of a gene in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 2000 bps flanking a promoter sequence of a gene in the epigenetically edited chromosome in a cell are demethylated as compared to the original state of the chromosome or the chromosome in a comparable cell not contacted with the epigenetic editor. In some embodiments, one single histone tail of histones bound to DNAs within 2000 bps flanking a promoter sequence of a gene in the epigenetically edited chromosome in a cell is demethylated as compared to the original state of the chromosome or the chromosome in a comparable cell not contacted with the epigenetic editor. In some embodiments, one single histone octamer bound to DNAs within 2000 bps flanking a promoter sequence of a gene in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 2000 bps flanking a promoter sequence of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of histones bound to DNAs within 2000 bps flanking a promoter sequence of a gene in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 2000 bps flanking a promoter sequence of a gene in the epigenetically edited chromosome in a cell are acetylated as compared to the original state of the chromosome or the chromosome in a comparable cell not contacted with the epigenetic editor. In some embodiments, one single histone tail of histones bound to DNAs within 2000 bps flanking a promoter sequence of a gene in the epigenetically edited chromosome in a cell is acetylated as compared to the original state of the chromosome or the chromosome in a comparable cell not contacted with the epigenetic editor. In some embodiments, one single histone octamer bound to DNAs within 2000 bps flanking a promoter sequence of a gene in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 2000 bps flanking a promoter sequence of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of histones bound to DNAs within 2000 bps flanking a promoter sequence of a gene in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 2000 bps flanking a promoter sequence of a gene in the epigenetically edited chromosome in a cell are deacetylated as compared to the original state of the chromosome or the chromosome in a comparable cell not contacted with the epigenetic editor. In some embodiments, one single histone tail of histones bound to DNAs within 2000 bps flanking a promoter sequence of a gene in the epigenetically edited chromosome in a cell is deacetylated as compared to the original state of the chromosome or the chromosome in a comparable cell not contacted with the epigenetic editor. In some embodiments, one single histone octamer bound to DNAs within 2000 bps flanking a promoter sequence of a gene in the epigenetically edited chromosome in a cell is deacetylated as compared to the original state of the chromosome or the chromosome in a comparable cell not contacted with the epigenetic editor.

In some embodiments, all CpG dinucleotides within 1500 bp flanking a promoter sequence of a gene in the epigenetically edited chromosome in a cell are methylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 550, 500, 600, 650, 700 or more CpG dinucleotides within 1500 bps flanking a promoter sequence of a gene in the epigenetically edited chromosome in a cell are methylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 1500 bps flanking a promoter sequence of a gene in the epigenetically edited chromosome in a cell are methylated as compared to the original state of the gene or the gene in a comparable cell not contacted with the epigenetic editor. In some embodiments, one single CpG dinucleotide within 1500 bps flanking a promoter sequence of a gene in the epigenetically edited chromosome in a cell is methylated as compared to the original state of the gene or the gene in a comparable cell not contacted with the epigenetic editor.

In some embodiments, all CpG dinucleotides within 1500 bps flanking a promoter sequence of a gene in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700 or more CpG dinucleotides within 1500 bps flanking a promoter sequence of a gene in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 1500 bps flanking a promoter sequence of a gene in the epigenetically edited chromosome in a cell are demethylated as compared to the original state of the gene or the gene in a comparable cell not contacted with the epigenetic editor. In some embodiments, one single CpG dinucleotide within 1500 bps flanking a promoter sequence of a gene in the epigenetically edited chromosome in a cell is demethylated as compared to the original state of the gene or the gene in a comparable cell not contacted with the epigenetic editor.

In some embodiments, all histone tails of histones bound to DNA nucleotides within 1500 bps flanking a promoter sequence of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more histone tails of histones bound to DNAs within 1500 bps flanking a promoter sequence of a gene in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1500 bps flanking a promoter sequence of a gene in the epigenetically edited chromosome in a cell are methylated as compared to the original state of the chromosome or the chromosome in a comparable cell not contacted with the epigenetic editor. In some embodiments, one single histone tail of histones bound to DNAs within 1500 bps flanking a promoter sequence of a gene in the epigenetically edited chromosome in a cell is methylated as compared to the original state of the chromosome or the chromosome in a comparable cell not contacted with the epigenetic editor. In some embodiments, one single histone octamer bound to DNAs within 1500 bps flanking a promoter sequence of a gene in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 1500 bps flanking a promoter sequence of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more histone tails of histones bound to DNAs within 1500 bps flanking a promoter sequence of a gene in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1500 bps flanking a promoter sequence of a gene in the epigenetically edited chromosome in a cell are demethylated as compared to the original state of the chromosome or the chromosome in a comparable cell not contacted with the epigenetic editor. In some embodiments, one single histone tail of histones bound to DNAs within 1500 bps flanking a promoter sequence of a gene in the epigenetically edited chromosome in a cell is demethylated as compared to the original state of the chromosome or the chromosome in a comparable cell not contacted with the epigenetic editor. In some embodiments, one single histone octamer bound to DNAs within 1500 bps flanking a promoter sequence of a gene in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 1500 bps flanking a promoter sequence of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more histone tails of histones bound to DNAs within 1500 bps flanking a promoter sequence of a gene in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1500 bps flanking a promoter sequence of a gene in the epigenetically edited chromosome in a cell are acetylated as compared to the original state of the chromosome or the chromosome in a comparable cell not contacted with the epigenetic editor. In some embodiments, one single histone tail of histones bound to DNAs within 1500 bps flanking a promoter sequence of a gene in the epigenetically edited chromosome in a cell is acetylated as compared to the original state of the chromosome or the chromosome in a comparable cell not contacted with the epigenetic editor. In some embodiments, one single histone octamer bound to DNAs within 1500 bps flanking a promoter sequence of a gene in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 1500 bps flanking a promoter sequence of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more histone tails of histones bound to DNAs within 1500 bps flanking a promoter sequence of a gene in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1500 bps flanking a promoter sequence of a gene in the epigenetically edited chromosome in a cell are deacetylated as compared to the original state of the chromosome or the chromosome in a comparable cell not contacted with the epigenetic editor. In some embodiments, one single histone tail of histones bound to DNAs within 1500 bps flanking a promoter sequence of a gene in the epigenetically edited chromosome in a cell is deacetylated as compared to the original state of the chromosome or the chromosome in a comparable cell not contacted with the epigenetic editor. In some embodiments, one single histone octamer bound to DNAs within 1500 bps flanking a promoter sequence of a gene in the epigenetically edited chromosome in a cell is deacetylated as compared to the original state of the chromosome or the chromosome in a comparable cell not contacted with the epigenetic editor.

In some embodiments, all CpG dinucleotides within 1000 bps flanking a promoter sequence of a gene in the epigenetically edited chromosome in a cell are methylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500 or more CpG dinucleotides within 1000 bps flanking a promoter sequence of a gene in the epigenetically edited chromosome in a cell are methylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 1000 bps flanking a promoter sequence of a gene in the epigenetically edited chromosome in a cell are methylated as compared to the original state of the gene or the gene in a comparable cell not contacted with the epigenetic editor. In some embodiments, one single CpG dinucleotide within 1000 bps flanking a promoter sequence of a gene in the epigenetically edited chromosome in a cell is methylated as compared to the original state of the gene or the gene in a comparable cell not contacted with the epigenetic editor.

In some embodiments, all CpG dinucleotides within 1000 bps flanking a promoter sequence of a gene in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500 or more CpG dinucleotides within 1000 bps flanking a promoter sequence of a gene in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 1000 bps flanking a promoter sequence of a gene in the epigenetically edited chromosome in a cell are demethylated as compared to the original state of the gene or the gene in a comparable cell not contacted with the epigenetic editor. In some embodiments, one single CpG dinucleotide within 1000 bps flanking a promoter sequence of a gene in the epigenetically edited chromosome in a cell is demethylated as compared to the original state of the gene or the gene in a comparable cell not contacted with the epigenetic editor.

In some embodiments, all histone tails of histones bound to DNA nucleotides within 1000 bps flanking a promoter sequence of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60 or more histone tails of histones bound to DNAs within 1000 bps flanking a promoter sequence of a gene in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1000 bps flanking a promoter sequence of a gene in the epigenetically edited chromosome in a cell are methylated as compared to the original state of the chromosome or the chromosome in a comparable cell not contacted with the epigenetic editor. In some embodiments, one single histone tail of histones bound to DNAs within 1000 bps flanking a promoter sequence of a gene in the epigenetically edited chromosome in a cell is methylated as compared to the original state of the chromosome or the chromosome in a comparable cell not contacted with the epigenetic editor. In some embodiments, one single histone octamer bound to DNAs within 1000 bps flanking a promoter sequence of a gene in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 1000 bps flanking a promoter sequence of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60 or more histone tails of histones bound to DNAs within 1000 bps flanking a promoter sequence of a gene in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55% 60%, 65%, 70%, 75%, 80%, 85%, 90%, 95%, 96%, 97%, 98%, or 99% of histone tails of histones bound to DNAs within 1000 bps flanking a promoter sequence of a gene in the epigenetically edited chromosome in a cell are demethylated as compared to the original state of the chromosome or the chromosome in a comparable cell not contacted with the epigenetic editor. In some embodiments, one single histone tail of histones bound to DNAs within 1000 bps flanking a promoter sequence of a gene in the epigenetically edited chromosome in a cell is demethylated as compared to the original state of the chromosome or the chromosome in a comparable cell not contacted with the epigenetic editor. In some embodiments, one single histone octamer bound to DNAs within 1000 bps flanking a promoter sequence of a gene in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 1000 bps flanking a promoter sequence of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60 or more histone tails of histones bound to DNAs within 1000 bps flanking a promoter sequence of a gene in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1000 bps flanking a promoter sequence of a gene in the epigenetically edited chromosome in a cell are acetylated as compared to the original state of the chromosome or the chromosome in a comparable cell not contacted with the epigenetic editor. In some embodiments, one single histone tail of histones bound to DNAs within 1000 bps flanking a promoter sequence of a gene in the epigenetically edited chromosome in a cell is acetylated as compared to the original state of the chromosome or the chromosome in a comparable cell not contacted with the epigenetic editor. In some embodiments, one single histone octamer bound to DNAs within 1000 bps flanking a promoter sequence of a gene in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 1000 bps flanking a promoter sequence of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60 or more histone tails of histones bound to DNAs within 1000 bps flanking a promoter sequence of a gene in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55% 60%, 65%, 70%, 75%, 80%, 85%, 90%, 95%, 96%, 97%, 98%, or 99% of histone tails of histones bound to DNAs within 1000 bps flanking a promoter sequence of a gene in the epigenetically edited chromosome in a cell are deacetylated as compared to the original state of the chromosome or the chromosome in a comparable cell not contacted with the epigenetic editor. In some embodiments, one single histone tail of histones bound to DNAs within 1000 bps flanking a promoter sequence of a gene in the epigenetically edited chromosome in a cell is deacetylated as compared to the original state of the chromosome or the chromosome in a comparable cell not contacted with the epigenetic editor. In some embodiments, one single histone octamer bound to DNAs within 1000 bps flanking a promoter sequence of a gene in the epigenetically edited chromosome in a cell is deacetylated as compared to the original state of the chromosome or the chromosome in a comparable cell not contacted with the epigenetic editor.

In some embodiments, all CpG dinucleotides within 500 bps flanking a promoter sequence of a gene in the epigenetically edited chromosome in a cell are methylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200 or more CpG dinucleotides within 500 bps flanking a promoter sequence of a gene in the epigenetically edited chromosome in a cell are methylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 500 bps flanking a promoter sequence of a gene in the epigenetically edited chromosome in a cell are methylated as compared to the original state of the gene or the gene in a comparable cell not contacted with the epigenetic editor. In some embodiments, one single CpG dinucleotide within 500 bps flanking a promoter sequence of a gene in the epigenetically edited chromosome in a cell is methylated as compared to the original state of the gene or the gene in a comparable cell not contacted with the epigenetic editor.

In some embodiments, all CpG dinucleotides within 500 bps flanking a promoter sequence of a gene in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200 or more CpG dinucleotides within 500 bps flanking a promoter sequence of a gene in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 500 bps flanking a promoter sequence of a gene in the epigenetically edited chromosome in a cell are demethylated as compared to the original state of the gene or the gene in a comparable cell not contacted with the epigenetic editor. In some embodiments, one single CpG dinucleotide within 500 bps flanking a promoter sequence of a gene in the epigenetically edited chromosome in a cell is demethylated as compared to the original state of the gene or the gene in a comparable cell not contacted with the epigenetic editor.

In some embodiments, all histone tails of histones bound to DNA nucleotides within 500 bps flanking a promoter sequence of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30 or more histone tails of histones bound to DNAs within 500 bps flanking a promoter sequence of a gene in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 500 bps flanking a promoter sequence of a gene in the epigenetically edited chromosome in a cell are methylated as compared to the original state of the chromosome or the chromosome in a comparable cell not contacted with the epigenetic editor. In some embodiments, one single histone tail of histones bound to DNAs within 500 bps flanking a promoter sequence of a gene in the epigenetically edited chromosome in a cell is methylated as compared to the original state of the chromosome or the chromosome in a comparable cell not contacted with the epigenetic editor. In some embodiments, one single histone octamer bound to DNAs within 500 bps flanking a promoter sequence of a gene in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 500 bps flanking a promoter sequence of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30 or more histone tails of histones bound to DNAs within 500 bps flanking a promoter sequence of a gene in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 500 bps flanking a promoter sequence of a gene in the epigenetically edited chromosome in a cell are demethylated as compared to the original state of the chromosome or the chromosome in a comparable cell not contacted with the epigenetic editor. In some embodiments, one single histone tail of histones bound to DNAs within 500 bps flanking a promoter sequence of a gene in the epigenetically edited chromosome in a cell is demethylated as compared to the original state of the chromosome or the chromosome in a comparable cell not contacted with the epigenetic editor. In some embodiments, one single histone octamer bound to DNAs within 500 bps flanking a promoter sequence of a gene in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 500 bps flanking a promoter sequence of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30 or more histone tails of histones bound to DNAs within 500 bps flanking a promoter sequence of a gene in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 500 bps flanking a promoter sequence of a gene in the epigenetically edited chromosome in a cell are acetylated as compared to the original state of the chromosome or the chromosome in a comparable cell not contacted with the epigenetic editor. In some embodiments, one single histone tail of histones bound to DNAs within 500 bps flanking a promoter sequence of a gene in the epigenetically edited chromosome in a cell is acetylated as compared to the original state of the chromosome or the chromosome in a comparable cell not contacted with the epigenetic editor. In some embodiments, one single histone octamer bound to DNAs within 500 bps flanking a promoter sequence of a gene in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 500 bps flanking a promoter sequence of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30 or more histone tails of histones bound to DNAs within 500 bps flanking a promoter sequence of a gene in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 500 bps flanking a promoter sequence of a gene in the epigenetically edited chromosome in a cell are deacetylated as compared to the original state of the chromosome or the chromosome in a comparable cell not contacted with the epigenetic editor. In some embodiments, one single histone tail of histones bound to DNAs within 500 bps flanking a promoter sequence of a gene in the epigenetically edited chromosome in a cell is deacetylated as compared to the original state of the chromosome or the chromosome in a comparable cell not contacted with the epigenetic editor. In some embodiments, one single histone octamer bound to DNAs within 500 bps flanking a promoter sequence of a gene in the epigenetically edited chromosome in a cell is deacetylated as compared to the original state of the chromosome or the chromosome in a comparable cell not contacted with the epigenetic editor.

In some embodiments, all CpG dinucleotides within 200 bps flanking a promoter sequence of a gene in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90 or more CpG dinucleotides within 200 bps flanking a promoter sequence of a gene in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 200 bps flanking a promoter sequence of a gene in the epigenetically edited chromosome in a cell are demethylated as compared to the original state of the gene or the gene in a comparable cell not contacted with the epigenetic editor. In some embodiments, one single CpG dinucleotide within 200 bps flanking a promoter sequence of a gene in the epigenetically edited chromosome in a cell is demethylated as compared to the original state of the gene or the gene in a comparable cell not contacted with the epigenetic editor.

In some embodiments, all histone tails of histones bound to DNA nucleotides within 200 bps flanking a promoter sequence of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20 or more histone tails of histones bound to DNAs within 200 bps flanking a promoter sequence of a gene in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 200 bps flanking a promoter sequence of a gene in the epigenetically edited chromosome in a cell are methylated as compared to the original state of the chromosome or the chromosome in a comparable cell not contacted with the epigenetic editor. In some embodiments, one single histone tail of histones bound to DNAs within 200 bps flanking a promoter sequence of a gene in the epigenetically edited chromosome in a cell is methylated as compared to the original state of the chromosome or the chromosome in a comparable cell not contacted with the epigenetic editor. In some embodiments, one single histone octamer bound to DNAs within 200 bps flanking a promoter sequence of a gene in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 200 bps flanking a promoter sequence of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20 or more histone tails of histones bound to DNAs within 200 bps flanking a promoter sequence of a gene in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 200 bps flanking a promoter sequence of a gene in the epigenetically edited chromosome in a cell are demethylated as compared to the original state of the chromosome or the chromosome in a comparable cell not contacted with the epigenetic editor. In some embodiments, one single histone tail of histones bound to DNAs within 200 bps flanking a promoter sequence of a gene in the epigenetically edited chromosome in a cell is demethylated as compared to the original state of the chromosome or the chromosome in a comparable cell not contacted with the epigenetic editor. In some embodiments, one single histone octamer bound to DNAs within 200 bps flanking a promoter sequence of a gene in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 200 bps flanking a promoter sequence of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20 or more histone tails of histones bound to DNAs within 200 bps flanking a promoter sequence of a gene in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 200 bps flanking a promoter sequence of a gene in the epigenetically edited chromosome in a cell are acetylated as compared to the original state of the chromosome or the chromosome in a comparable cell not contacted with the epigenetic editor. In some embodiments, one single histone tail of histones bound to DNAs within 200 bps flanking a promoter sequence of a gene in the epigenetically edited chromosome in a cell is acetylated as compared to the original state of the chromosome or the chromosome in a comparable cell not contacted with the epigenetic editor. In some embodiments, one single histone octamer bound to DNAs within 200 bps flanking a promoter sequence of a gene in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 200 bps flanking a promoter sequence of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20 or more histone tails of histones bound to DNAs within 200 bps flanking a promoter sequence of a gene in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 200 bps flanking a promoter sequence of a gene in the epigenetically edited chromosome in a cell are deacetylated as compared to the original state of the chromosome or the chromosome in a comparable cell not contacted with the epigenetic editor. In some embodiments, one single histone tail of histones bound to DNAs within 200 bps flanking a promoter sequence of a gene in the epigenetically edited chromosome in a cell is deacetylated as compared to the original state of the chromosome or the chromosome in a comparable cell not contacted with the epigenetic editor. In some embodiments, one single histone octamer bound to DNAs within 200 bps flanking a promoter sequence of a gene in the epigenetically edited chromosome in a cell is deacetylated as compared to the original state of the chromosome or the chromosome in a comparable cell not contacted with the epigenetic editor.

In some embodiments, all CpG dinucleotides within 2000 bps flanking a enhancer sequence, an isolator sequence, or a CTCF binding sequence of a gene in the epigenetically edited chromosome in a cell are methylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700 or more CpG dinucleotides within 2000 bps flanking a enhancer sequence, an isolator sequence, or a CTCF binding sequence of a gene in the epigenetically edited chromosome in a cell are methylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 2000 bps flanking a enhancer sequence, an isolator sequence, or a CTCF binding sequence of a gene in the epigenetically edited chromosome in a cell are methylated as compared to the original state of the gene or the gene in a comparable cell not contacted with the epigenetic editor. In some embodiments, one single CpG dinucleotide within 2000 bps flanking a enhancer sequence, an isolator sequence, or a CTCF binding sequence of a gene in the epigenetically edited chromosome in a cell is methylated as compared to the original state of the gene or the gene in a comparable cell not contacted with the epigenetic editor.

In some embodiments, all CpG dinucleotides within 2000 bps flanking a enhancer sequence, isolator sequence, or CTCF binding sequence of a gene in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700 or more CpG dinucleotides within 2000 bps flanking a enhancer sequence, isolator sequence, or CTCF binding sequence of a gene in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 2000 bps flanking a enhancer sequence, isolator sequence, or CTCF binding sequence of a gene in the epigenetically edited chromosome in a cell are demethylated as compared to the original state of the gene or the gene in a comparable cell not contacted with the epigenetic editor. In some embodiments, one single CpG dinucleotide within 2000 bps flanking a enhancer sequence, isolator sequence, or CTCF binding sequence of a gene in the epigenetically edited chromosome in a cell is demethylated as compared to the original state of the gene or the gene in a comparable cell not contacted with the epigenetic editor.

In some embodiments, all histone tails of histones bound to DNA nucleotides within 2000 bps flanking a enhancer sequence, isolator sequence, or CTCF binding sequence of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of histones bound to DNAs within 2000 bps flanking a enhancer sequence, isolator sequence, or CTCF binding sequence of a gene in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 2000 bps flanking a enhancer sequence, isolator sequence, or CTCF binding sequence of a gene in the epigenetically edited chromosome in a cell are methylated as compared to the original state of the chromosome or the chromosome in a comparable cell not contacted with the epigenetic editor. In some embodiments, one single histone tail of histones bound to DNAs within 2000 bps flanking a enhancer sequence, isolator sequence, or CTCF binding sequence of a gene in the epigenetically edited chromosome in a cell is methylated as compared to the original state of the chromosome or the chromosome in a comparable cell not contacted with the epigenetic editor. In some embodiments, one single histone octamer bound to DNAs within 2000 bps flanking a enhancer sequence, isolator sequence, or CTCF binding sequence of a gene in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 2000 bps flanking a enhancer sequence, isolator sequence, or CTCF binding sequence of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of histones bound to DNAs within 2000 bps flanking a enhancer sequence, isolator sequence, or CTCF binding sequence of a gene in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 2000 bps flanking a enhancer sequence, isolator sequence, or CTCF binding sequence of a gene in the epigenetically edited chromosome in a cell are demethylated as compared to the original state of the chromosome or the chromosome in a comparable cell not contacted with the epigenetic editor. In some embodiments, one single histone tail of histones bound to DNAs within 2000 bps flanking a enhancer sequence, isolator sequence, or CTCF binding sequence of a gene in the epigenetically edited chromosome in a cell is demethylated as compared to the original state of the chromosome or the chromosome in a comparable cell not contacted with the epigenetic editor. In some embodiments, one single histone octamer bound to DNAs within 2000 bps flanking a enhancer sequence, isolator sequence, or CTCF binding sequence of a gene in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 2000 bps flanking a enhancer sequence, isolator sequence, or CTCF binding sequence of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of histones bound to DNAs within 2000 bps flanking a enhancer sequence, isolator sequence, or CTCF binding sequence of a gene in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 2000 bps flanking a enhancer sequence, isolator sequence, or CTCF binding sequence of a gene in the epigenetically edited chromosome in a cell are acetylated as compared to the original state of the chromosome or the chromosome in a comparable cell not contacted with the epigenetic editor. In some embodiments, one single histone tail of histones bound to DNAs within 2000 bps flanking a enhancer sequence, isolator sequence, or CTCF binding sequence of a gene in the epigenetically edited chromosome in a cell is acetylated as compared to the original state of the chromosome or the chromosome in a comparable cell not contacted with the epigenetic editor. In some embodiments, one single histone octamer bound to DNAs within 2000 bps flanking a enhancer sequence, isolator sequence, or CTCF binding sequence of a gene in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 2000 bps flanking a enhancer sequence, isolator sequence, or CTCF binding sequence of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of histones bound to DNAs within 2000 bps flanking a enhancer sequence, isolator sequence, or CTCF binding sequence of a gene in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 2000 bps flanking a enhancer sequence, isolator sequence, or CTCF binding sequence of a gene in the epigenetically edited chromosome in a cell are deacetylated as compared to the original state of the chromosome or the chromosome in a comparable cell not contacted with the epigenetic editor. In some embodiments, one single histone tail of histones bound to DNAs within 2000 bps flanking a enhancer sequence, isolator sequence, or CTCF binding sequence of a gene in the epigenetically edited chromosome in a cell is deacetylated as compared to the original state of the chromosome or the chromosome in a comparable cell not contacted with the epigenetic editor. In some embodiments, one single histone octamer bound to DNAs within 2000 bps flanking a enhancer sequence, isolator sequence, or CTCF binding sequence of a gene in the epigenetically edited chromosome in a cell is deacetylated as compared to the original state of the chromosome or the chromosome in a comparable cell not contacted with the epigenetic editor.

In some embodiments, all CpG dinucleotides within 1500 bp flanking a enhancer sequence, an isolator sequence, or a CTCF binding sequence of a gene in the epigenetically edited chromosome in a cell are methylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 550, 500, 600, 650, 700 or more CpG dinucleotides within 1500 bps flanking a enhancer sequence, an isolator sequence, or a CTCF binding sequence of a gene in the epigenetically edited chromosome in a cell are methylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 1500 bps flanking a enhancer sequence, an isolator sequence, or a CTCF binding sequence of a gene in the epigenetically edited chromosome in a cell are methylated as compared to the original state of the gene or the gene in a comparable cell not contacted with the epigenetic editor. In some embodiments, one single CpG dinucleotide within 1500 bps flanking a enhancer sequence, an isolator sequence, or a CTCF binding sequence of a gene in the epigenetically edited chromosome in a cell is methylated as compared to the original state of the gene or the gene in a comparable cell not contacted with the epigenetic editor.

In some embodiments, all CpG dinucleotides within 1500 bps flanking a enhancer sequence, isolator sequence, or CTCF binding sequence of a gene in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700 or more CpG dinucleotides within 1500 bps flanking a enhancer sequence, isolator sequence, or CTCF binding sequence of a gene in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 1500 bps flanking a enhancer sequence, isolator sequence, or CTCF binding sequence of a gene in the epigenetically edited chromosome in a cell are demethylated as compared to the original state of the gene or the gene in a comparable cell not contacted with the epigenetic editor. In some embodiments, one single CpG dinucleotide within 1500 bps flanking a enhancer sequence, isolator sequence, or CTCF binding sequence of a gene in the epigenetically edited chromosome in a cell is demethylated as compared to the original state of the gene or the gene in a comparable cell not contacted with the epigenetic editor.

In some embodiments, all histone tails of histones bound to DNA nucleotides within 1500 bps flanking a enhancer sequence, isolator sequence, or CTCF binding sequence of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more histone tails of histones bound to DNAs within 1500 bps flanking a enhancer sequence, isolator sequence, or CTCF binding sequence of a gene in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1500 bps flanking a enhancer sequence, isolator sequence, or CTCF binding sequence of a gene in the epigenetically edited chromosome in a cell are methylated as compared to the original state of the chromosome or the chromosome in a comparable cell not contacted with the epigenetic editor. In some embodiments, one single histone tail of histones bound to DNAs within 1500 bps flanking a enhancer sequence, isolator sequence, or CTCF binding sequence of a gene in the epigenetically edited chromosome in a cell is methylated as compared to the original state of the chromosome or the chromosome in a comparable cell not contacted with the epigenetic editor. In some embodiments, one single histone octamer bound to DNAs within 1500 bps flanking a enhancer sequence, isolator sequence, or CTCF binding sequence of a gene in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 1500 bps flanking a enhancer sequence, isolator sequence, or CTCF binding sequence of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more histone tails of histones bound to DNAs within 1500 bps flanking a enhancer sequence, isolator sequence, or CTCF binding sequence of a gene in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1500 bps flanking a enhancer sequence, isolator sequence, or CTCF binding sequence of a gene in the epigenetically edited chromosome in a cell are demethylated as compared to the original state of the chromosome or the chromosome in a comparable cell not contacted with the epigenetic editor. In some embodiments, one single histone tail of histones bound to DNAs within 1500 bps flanking a enhancer sequence, isolator sequence, or CTCF binding sequence of a gene in the epigenetically edited chromosome in a cell is demethylated as compared to the original state of the chromosome or the chromosome in a comparable cell not contacted with the epigenetic editor. In some embodiments, one single histone octamer bound to DNAs within 1500 bps flanking a enhancer sequence, isolator sequence, or CTCF binding sequence of a gene in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 1500 bps flanking a enhancer sequence, isolator sequence, or CTCF binding sequence of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more histone tails of histones bound to DNAs within 1500 bps flanking a enhancer sequence, isolator sequence, or CTCF binding sequence of a gene in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1500 bps flanking a enhancer sequence, isolator sequence, or CTCF binding sequence of a gene in the epigenetically edited chromosome in a cell are acetylated as compared to the original state of the chromosome or the chromosome in a comparable cell not contacted with the epigenetic editor. In some embodiments, one single histone tail of histones bound to DNAs within 1500 bps flanking a enhancer sequence, isolator sequence, or CTCF binding sequence of a gene in the epigenetically edited chromosome in a cell is acetylated as compared to the original state of the chromosome or the chromosome in a comparable cell not contacted with the epigenetic editor. In some embodiments, one single histone octamer bound to DNAs within 1500 bps flanking a enhancer sequence, isolator sequence, or CTCF binding sequence of a gene in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 1500 bps flanking a enhancer sequence, isolator sequence, or CTCF binding sequence of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more histone tails of histones bound to DNAs within 1500 bps flanking a enhancer sequence, isolator sequence, or CTCF binding sequence of a gene in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1500 bps flanking a enhancer sequence, isolator sequence, or CTCF binding sequence of a gene in the epigenetically edited chromosome in a cell are deacetylated as compared to the original state of the chromosome or the chromosome in a comparable cell not contacted with the epigenetic editor. In some embodiments, one single histone tail of histones bound to DNAs within 1500 bps flanking a enhancer sequence, isolator sequence, or CTCF binding sequence of a gene in the epigenetically edited chromosome in a cell is deacetylated as compared to the original state of the chromosome or the chromosome in a comparable cell not contacted with the epigenetic editor. In some embodiments, one single histone octamer bound to DNAs within 1500 bps flanking a enhancer sequence, isolator sequence, or CTCF binding sequence of a gene in the epigenetically edited chromosome in a cell is deacetylated as compared to the original state of the chromosome or the chromosome in a comparable cell not contacted with the epigenetic editor.

In some embodiments, all CpG dinucleotides within 1000 bps flanking a enhancer sequence, an isolator sequence, or a CTCF binding sequence of a gene in the epigenetically edited chromosome in a cell are methylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500 or more CpG dinucleotides within 1000 bps flanking a enhancer sequence, an isolator sequence, or a CTCF binding sequence of a gene in the epigenetically edited chromosome in a cell are methylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 1000 bps flanking a enhancer sequence, an isolator sequence, or a CTCF binding sequence of a gene in the epigenetically edited chromosome in a cell are methylated as compared to the original state of the gene or the gene in a comparable cell not contacted with the epigenetic editor. In some embodiments, one single CpG dinucleotide within 1000 bps flanking a enhancer sequence, an isolator sequence, or a CTCF binding sequence of a gene in the epigenetically edited chromosome in a cell is methylated as compared to the original state of the gene or the gene in a comparable cell not contacted with the epigenetic editor.

In some embodiments, all CpG dinucleotides within 1000 bps flanking a enhancer sequence, isolator sequence, or CTCF binding sequence of a gene in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500 or more CpG dinucleotides within 1000 bps flanking a enhancer sequence, isolator sequence, or CTCF binding sequence of a gene in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 1000 bps flanking a enhancer sequence, isolator sequence, or CTCF binding sequence of a gene in the epigenetically edited chromosome in a cell are demethylated as compared to the original state of the gene or the gene in a comparable cell not contacted with the epigenetic editor. In some embodiments, one single CpG dinucleotide within 1000 bps flanking a enhancer sequence, isolator sequence, or CTCF binding sequence of a gene in the epigenetically edited chromosome in a cell is demethylated as compared to the original state of the gene or the gene in a comparable cell not contacted with the epigenetic editor.

In some embodiments, all histone tails of histones bound to DNA nucleotides within 1000 bps flanking a enhancer sequence, isolator sequence, or CTCF binding sequence of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60 or more histone tails of histones bound to DNAs within 1000 bps flanking a enhancer sequence, isolator sequence, or CTCF binding sequence of a gene in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1000 bps flanking a enhancer sequence, isolator sequence, or CTCF binding sequence of a gene in the epigenetically edited chromosome in a cell are methylated as compared to the original state of the chromosome or the chromosome in a comparable cell not contacted with the epigenetic editor. In some embodiments, one single histone tail of histones bound to DNAs within 1000 bps flanking a enhancer sequence, isolator sequence, or CTCF binding sequence of a gene in the epigenetically edited chromosome in a cell is methylated as compared to the original state of the chromosome or the chromosome in a comparable cell not contacted with the epigenetic editor. In some embodiments, one single histone octamer bound to DNAs within 1000 bps flanking a enhancer sequence, isolator sequence, or CTCF binding sequence of a gene in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 1000 bps flanking a enhancer sequence, isolator sequence, or CTCF binding sequence of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60 or more histone tails of histones bound to DNAs within 1000 bps flanking a enhancer sequence, isolator sequence, or CTCF binding sequence of a gene in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1000 bps flanking a enhancer sequence, isolator sequence, or CTCF binding sequence of a gene in the epigenetically edited chromosome in a cell are demethylated as compared to the original state of the chromosome or the chromosome in a comparable cell not contacted with the epigenetic editor. In some embodiments, one single histone tail of histones bound to DNAs within 1000 bps flanking a enhancer sequence, isolator sequence, or CTCF binding sequence of a gene in the epigenetically edited chromosome in a cell is demethylated as compared to the original state of the chromosome or the chromosome in a comparable cell not contacted with the epigenetic editor. In some embodiments, one single histone octamer bound to DNAs within 1000 bps flanking a enhancer sequence, isolator sequence, or CTCF binding sequence of a gene in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 1000 bps flanking a enhancer sequence, isolator sequence, or CTCF binding sequence of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60 or more histone tails of histones bound to DNAs within 1000 bps flanking a enhancer sequence, isolator sequence, or CTCF binding sequence of a gene in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1000 bps flanking a enhancer sequence, isolator sequence, or CTCF binding sequence of a gene in the epigenetically edited chromosome in a cell are acetylated as compared to the original state of the chromosome or the chromosome in a comparable cell not contacted with the epigenetic editor. In some embodiments, one single histone tail of histones bound to DNAs within 1000 bps flanking a enhancer sequence, isolator sequence, or CTCF binding sequence of a gene in the epigenetically edited chromosome in a cell is acetylated as compared to the original state of the chromosome or the chromosome in a comparable cell not contacted with the epigenetic editor. In some embodiments, one single histone octamer bound to DNAs within 1000 bps flanking a enhancer sequence, isolator sequence, or CTCF binding sequence of a gene in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 1000 bps flanking a enhancer sequence, isolator sequence, or CTCF binding sequence of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60 or more histone tails of histones bound to DNAs within 1000 bps flanking a enhancer sequence, isolator sequence, or CTCF binding sequence of a gene in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1000 bps flanking a enhancer sequence, isolator sequence, or CTCF binding sequence of a gene in the epigenetically edited chromosome in a cell are deacetylated as compared to the original state of the chromosome or the chromosome in a comparable cell not contacted with the epigenetic editor. In some embodiments, one single histone tail of histones bound to DNAs within 1000 bps flanking a enhancer sequence, isolator sequence, or CTCF binding sequence of a gene in the epigenetically edited chromosome in a cell is deacetylated as compared to the original state of the chromosome or the chromosome in a comparable cell not contacted with the epigenetic editor. In some embodiments, one single histone octamer bound to DNAs within 1000 bps flanking a enhancer sequence, isolator sequence, or CTCF binding sequence of a gene in the epigenetically edited chromosome in a cell is deacetylated as compared to the original state of the chromosome or the chromosome in a comparable cell not contacted with the epigenetic editor.

In some embodiments, all CpG dinucleotides within 500 bps flanking a enhancer sequence, an isolator sequence, or a CTCF binding sequence of a gene in the epigenetically edited chromosome in a cell are methylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200 or more CpG dinucleotides within 500 bps flanking a enhancer sequence, an isolator sequence, or a CTCF binding sequence of a gene in the epigenetically edited chromosome in a cell are methylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 500 bps flanking a enhancer sequence, an isolator sequence, or a CTCF binding sequence of a gene in the epigenetically edited chromosome in a cell are methylated as compared to the original state of the gene or the gene in a comparable cell not contacted with the epigenetic editor. In some embodiments, one single CpG dinucleotide within 500 bps flanking a enhancer sequence, an isolator sequence, or a CTCF binding sequence of a gene in the epigenetically edited chromosome in a cell is methylated as compared to the original state of the gene or the gene in a comparable cell not contacted with the epigenetic editor.

In some embodiments, all CpG dinucleotides within 500 bps flanking a enhancer sequence, isolator sequence, or CTCF binding sequence of a gene in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200 or more CpG dinucleotides within 500 bps flanking a enhancer sequence, isolator sequence, or CTCF binding sequence of a gene in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 6%, 70%, 75%, 80%, 85%, 90%, 95%, 96%, 97%, 98%, or 99% of CpG dinucleotides within 500 bps flanking a enhancer sequence, isolator sequence, or CTCF binding sequence of a gene in the epigenetically edited chromosome in a cell are demethylated as compared to the original state of the gene or the gene in a comparable cell not contacted with the epigenetic editor. In some embodiments, one single CpG dinucleotide within 500 bps flanking a enhancer sequence, isolator sequence, or CTCF binding sequence of a gene in the epigenetically edited chromosome in a cell is demethylated as compared to the original state of the gene or the gene in a comparable cell not contacted with the epigenetic editor.

In some embodiments, all histone tails of histones bound to DNA nucleotides within 500 bps flanking a enhancer sequence, isolator sequence, or CTCF binding sequence of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30 or more histone tails of histones bound to DNAs within 500 bps flanking a enhancer sequence, isolator sequence, or CTCF binding sequence of a gene in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 500 bps flanking a enhancer sequence, isolator sequence, or CTCF binding sequence of a gene in the epigenetically edited chromosome in a cell are methylated as compared to the original state of the chromosome or the chromosome in a comparable cell not contacted with the epigenetic editor. In some embodiments, one single histone tail of histones bound to DNAs within 500 bps flanking a enhancer sequence, isolator sequence, or CTCF binding sequence of a gene in the epigenetically edited chromosome in a cell is methylated as compared to the original state of the chromosome or the chromosome in a comparable cell not contacted with the epigenetic editor. In some embodiments, one single histone octamer bound to DNAs within 500 bps flanking a enhancer sequence, isolator sequence, or CTCF binding sequence of a gene in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 500 bps flanking a enhancer sequence, isolator sequence, or CTCF binding sequence of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30 or more histone tails of histones bound to DNAs within 500 bps flanking a enhancer sequence, isolator sequence, or CTCF binding sequence of a gene in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 500 bps flanking a enhancer sequence, isolator sequence, or CTCF binding sequence of a gene in the epigenetically edited chromosome in a cell are demethylated as compared to the original state of the chromosome or the chromosome in a comparable cell not contacted with the epigenetic editor. In some embodiments, one single histone tail of histones bound to DNAs within 500 bps flanking a enhancer sequence, isolator sequence, or CTCF binding sequence of a gene in the epigenetically edited chromosome in a cell is demethylated as compared to the original state of the chromosome or the chromosome in a comparable cell not contacted with the epigenetic editor. In some embodiments, one single histone octamer bound to DNAs within 500 bps flanking a enhancer sequence, isolator sequence, or CTCF binding sequence of a gene in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 500 bps flanking a enhancer sequence, isolator sequence, or CTCF binding sequence of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30 or more histone tails of histones bound to DNAs within 500 bps flanking a enhancer sequence, isolator sequence, or CTCF binding sequence of a gene in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 500 bps flanking a enhancer sequence, isolator sequence, or CTCF binding sequence of a gene in the epigenetically edited chromosome in a cell are acetylated as compared to the original state of the chromosome or the chromosome in a comparable cell not contacted with the epigenetic editor. In some embodiments, one single histone tail of histones bound to DNAs within 500 bps flanking a enhancer sequence, isolator sequence, or CTCF binding sequence of a gene in the epigenetically edited chromosome in a cell is acetylated as compared to the original state of the chromosome or the chromosome in a comparable cell not contacted with the epigenetic editor. In some embodiments, one single histone octamer bound to DNAs within 500 bps flanking a enhancer sequence, isolator sequence, or CTCF binding sequence of a gene in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 500 bps flanking a enhancer sequence, isolator sequence, or CTCF binding sequence of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30 or more histone tails of histones bound to DNAs within 500 bps flanking a enhancer sequence, isolator sequence, or CTCF binding sequence of a gene in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 500 bps flanking a enhancer sequence, isolator sequence, or CTCF binding sequence of a gene in the epigenetically edited chromosome in a cell are deacetylated as compared to the original state of the chromosome or the chromosome in a comparable cell not contacted with the epigenetic editor. In some embodiments, one single histone tail of histones bound to DNAs within 500 bps flanking a enhancer sequence, isolator sequence, or CTCF binding sequence of a gene in the epigenetically edited chromosome in a cell is deacetylated as compared to the original state of the chromosome or the chromosome in a comparable cell not contacted with the epigenetic editor. In some embodiments, one single histone octamer bound to DNAs within 500 bps flanking a enhancer sequence, isolator sequence, or CTCF binding sequence of a gene in the epigenetically edited chromosome in a cell is deacetylated as compared to the original state of the chromosome or the chromosome in a comparable cell not contacted with the epigenetic editor.

In some embodiments, all CpG dinucleotides within 200 bps flanking a enhancer sequence, isolator sequence, or CTCF binding site of a gene in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90 or more CpG dinucleotides within 200 bps flanking a enhancer sequence, isolator sequence, or CTCF binding site of a gene in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 200 bps flanking a enhancer sequence, isolator sequence, or CTCF binding site of a gene in the epigenetically edited chromosome in a cell are demethylated as compared to the original state of the gene or the gene in a comparable cell not contacted with the epigenetic editor. In some embodiments, one single CpG dinucleotide within 200 bps flanking a enhancer sequence, isolator sequence, or CTCF binding site of a gene in the epigenetically edited chromosome in a cell is demethylated as compared to the original state of the gene or the gene in a comparable cell not contacted with the epigenetic editor.

In some embodiments, all histone tails of histones bound to DNA nucleotides within 200 bps flanking a enhancer sequence, isolator sequence, or CTCF binding site of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20 or more histone tails of histones bound to DNAs within 200 bps flanking a enhancer sequence, isolator sequence, or CTCF binding site of a gene in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 200 bps flanking a enhancer sequence, isolator sequence, or CTCF binding site of a gene in the epigenetically edited chromosome in a cell are methylated as compared to the original state of the chromosome or the chromosome in a comparable cell not contacted with the epigenetic editor. In some embodiments, one single histone tail of histones bound to DNAs within 200 bps flanking a enhancer sequence, isolator sequence, or CTCF binding site of a gene in the epigenetically edited chromosome in a cell is methylated as compared to the original state of the chromosome or the chromosome in a comparable cell not contacted with the epigenetic editor. In some embodiments, one single histone octamer bound to DNAs within 200 bps flanking a enhancer sequence, isolator sequence, or CTCF binding site of a gene in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 200 bps flanking a enhancer sequence, isolator sequence, or CTCF binding site of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20 or more histone tails of histones bound to DNAs within 200 bps flanking a enhancer sequence, isolator sequence, or CTCF binding site of a gene in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 200 bps flanking a enhancer sequence, isolator sequence, or CTCF binding site of a gene in the epigenetically edited chromosome in a cell are demethylated as compared to the original state of the chromosome or the chromosome in a comparable cell not contacted with the epigenetic editor. In some embodiments, one single histone tail of histones bound to DNAs within 200 bps flanking a enhancer sequence, isolator sequence, or CTCF binding site of a gene in the epigenetically edited chromosome in a cell is demethylated as compared to the original state of the chromosome or the chromosome in a comparable cell not contacted with the epigenetic editor. In some embodiments, one single histone octamer bound to DNAs within 200 bps flanking a enhancer sequence, isolator sequence, or CTCF binding site of a gene in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 200 bps flanking a enhancer sequence, isolator sequence, or CTCF binding site of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20 or more histone tails of histones bound to DNAs within 200 bps flanking a enhancer sequence, isolator sequence, or CTCF binding site of a gene in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 200 bps flanking a enhancer sequence, isolator sequence, or CTCF binding site of a gene in the epigenetically edited chromosome in a cell are acetylated as compared to the original state of the chromosome or the chromosome in a comparable cell not contacted with the epigenetic editor. In some embodiments, one single histone tail of histones bound to DNAs within 200 bps flanking a enhancer sequence, isolator sequence, or CTCF binding site of a gene in the epigenetically edited chromosome in a cell is acetylated as compared to the original state of the chromosome or the chromosome in a comparable cell not contacted with the epigenetic editor. In some embodiments, one single histone octamer bound to DNAs within 200 bps flanking a enhancer sequence, isolator sequence, or CTCF binding site of a gene in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 200 bps flanking a enhancer sequence, isolator sequence, or CTCF binding site of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20 or more histone tails of histones bound to DNAs within 200 bps flanking a enhancer sequence, isolator sequence, or CTCF binding site of a gene in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 200 bps flanking a enhancer sequence, isolator sequence, or CTCF binding site of a gene in the epigenetically edited chromosome in a cell are deacetylated as compared to the original state of the chromosome or the chromosome in a comparable cell not contacted with the epigenetic editor. In some embodiments, one single histone tail of histones bound to DNAs within 200 bps flanking a enhancer sequence, isolator sequence, or CTCF binding site of a gene in the epigenetically edited chromosome in a cell is deacetylated as compared to the original state of the chromosome or the chromosome in a comparable cell not contacted with the epigenetic editor. In some embodiments, one single histone octamer bound to DNAs within 200 bps flanking a enhancer sequence, isolator sequence, or CTCF binding site of a gene in the epigenetically edited chromosome in a cell is deacetylated as compared to the original state of the chromosome or the chromosome in a comparable cell not contacted with the epigenetic editor.

In some embodiments, an epigenetically modified chromosome results from contacting a chromosome with an epigenetic editor as described herein. For example, an epigenetic editor may target a target sequence in a target gene in the chromosome and alter an epigenetic modification state of one or more nucleotides or one or more histone tails in the chromosome. The epigenetic modification placed or removed by the epigenetic editor may be in close proximity to the target sequence, or may be 50, 100, 200, 300, 400, 500, 600, 700, 800, 900, 1000, 1100, 1200, 1300, 1400, 1500, 1600, 1700, 1800, 1900, 2000, 2500, 3000 or more base pairs upstream or downstream of such target sequence. in some embodiments, the epigenetic editor initiates an epigenetic modification, e.g. DNA methylation, at one or more nucleotides in close proximity to the target sequence. The initial epigenetic modification may spread to nucleotides or histones upstream or downstream of the target sequence, for example, 50, 100, 200, 300, 400, 500, 600, 700, 800, 900, 1000, 1100, 1200, 1300, 1400, 1500, 1600, 1700, 1800, 1900, 2000, 2500, 3000 or more base pairs upstream or downstream of such target sequence.

In some embodiments, all CpG dinucleotides within 2000 bps flanking a target sequence in the epigenetically edited chromosome in a cell are methylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700 or more CpG dinucleotides within 2000 bps flanking a target sequence in the epigenetically edited chromosome in a cell are methylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 2000 bps flanking a target sequence in the epigenetically edited chromosome in a cell are methylated as compared to the original state of the gene or the gene in a comparable cell not contacted with the epigenetic editor. In some embodiments, one single CpG dinucleotide within 2000 bps flanking a target sequence in the epigenetically edited chromosome in a cell is methylated as compared to the original state of the gene or the gene in a comparable cell not contacted with the epigenetic editor.

In some embodiments, all CpG dinucleotides within 2000 bps flanking a target sequence in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700 or more CpG dinucleotides within 2000 bps flanking a target sequence in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 2000 bps flanking a target sequence in the epigenetically edited chromosome in a cell are demethylated as compared to the original state of the gene or the gene in a comparable cell not contacted with the epigenetic editor. In some embodiments, one single CpG dinucleotide within 2000 bps flanking a target sequence in the epigenetically edited chromosome in a cell is demethylated as compared to the original state of the gene or the gene in a comparable cell not contacted with the epigenetic editor.

In some embodiments, all histone tails of histones bound to DNA nucleotides within 2000 bps flanking a promoter sequence of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of histones bound to DNAs within 2000 bps flanking a target sequence in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 2000 bps flanking a target sequence in the epigenetically edited chromosome in a cell are methylated as compared to the original state of the chromosome or the chromosome in a comparable cell not contacted with the epigenetic editor. In some embodiments, one single histone tail of histones bound to DNAs within 2000 bps flanking a target sequence in the epigenetically edited chromosome in a cell is methylated as compared to the original state of the chromosome or the chromosome in a comparable cell not contacted with the epigenetic editor. In some embodiments, one single histone octamer bound to DNAs within 2000 bps flanking a target sequence in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 2000 bps flanking a promoter sequence of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of histones bound to DNAs within 2000 bps flanking a target sequence in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 2000 bps flanking a target sequence in the epigenetically edited chromosome in a cell are demethylated as compared to the original state of the chromosome or the chromosome in a comparable cell not contacted with the epigenetic editor. In some embodiments, one single histone tail of histones bound to DNAs within 2000 bps flanking a target sequence in the epigenetically edited chromosome in a cell is demethylated as compared to the original state of the chromosome or the chromosome in a comparable cell not contacted with the epigenetic editor. In some embodiments, one single histone octamer bound to DNAs within 2000 bps flanking a target sequence in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 2000 bps flanking a promoter sequence of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of histones bound to DNAs within 2000 bps flanking a target sequence in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 2000 bps flanking a target sequence in the epigenetically edited chromosome in a cell are acetylated as compared to the original state of the chromosome or the chromosome in a comparable cell not contacted with the epigenetic editor. In some embodiments, one single histone tail of histones bound to DNAs within 2000 bps flanking a target sequence in the epigenetically edited chromosome in a cell is acetylated as compared to the original state of the chromosome or the chromosome in a comparable cell not contacted with the epigenetic editor. In some embodiments, one single histone octamer bound to DNAs within 2000 bps flanking a target sequence in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 2000 bps flanking a promoter sequence of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or more histone tails of histones bound to DNAs within 2000 bps flanking a target sequence in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 2000 bps flanking a target sequence in the epigenetically edited chromosome in a cell are deacetylated as compared to the original state of the chromosome or the chromosome in a comparable cell not contacted with the epigenetic editor. In some embodiments, one single histone tail of histones bound to DNAs within 2000 bps flanking a target sequence in the epigenetically edited chromosome in a cell is deacetylated as compared to the original state of the chromosome or the chromosome in a comparable cell not contacted with the epigenetic editor. In some embodiments, one single histone octamer bound to DNAs within 2000 bps flanking a target sequence in the epigenetically edited chromosome in a cell is deacetylated as compared to the original state of the chromosome or the chromosome in a comparable cell not contacted with the epigenetic editor.

In some embodiments, all CpG dinucleotides within 1500 bp flanking a target sequence in the epigenetically edited chromosome in a cell are methylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 550, 500, 600, 650, 700 or more CpG dinucleotides within 1500 bps flanking a target sequence in the epigenetically edited chromosome in a cell are methylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 1500 bps flanking a target sequence in the epigenetically edited chromosome in a cell are methylated as compared to the original state of the gene or the gene in a comparable cell not contacted with the epigenetic editor. In some embodiments, one single CpG dinucleotide within 1500 bps flanking a target sequence in the epigenetically edited chromosome in a cell is methylated as compared to the original state of the gene or the gene in a comparable cell not contacted with the epigenetic editor.

In some embodiments, all CpG dinucleotides within 1500 bps flanking a target sequence in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700 or more CpG dinucleotides within 1500 bps flanking a target sequence in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 1500 bps flanking a target sequence in the epigenetically edited chromosome in a cell are demethylated as compared to the original state of the gene or the gene in a comparable cell not contacted with the epigenetic editor. In some embodiments, one single CpG dinucleotide within 1500 bps flanking a target sequence in the epigenetically edited chromosome in a cell is demethylated as compared to the original state of the gene or the gene in a comparable cell not contacted with the epigenetic editor.

In some embodiments, all histone tails of histones bound to DNA nucleotides within 1500 bps flanking a promoter sequence of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more histone tails of histones bound to DNAs within 1500 bps flanking a target sequence in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1500 bps flanking a target sequence in the epigenetically edited chromosome in a cell are methylated as compared to the original state of the chromosome or the chromosome in a comparable cell not contacted with the epigenetic editor. In some embodiments, one single histone tail of histones bound to DNAs within 1500 bps flanking a target sequence in the epigenetically edited chromosome in a cell is methylated as compared to the original state of the chromosome or the chromosome in a comparable cell not contacted with the epigenetic editor. In some embodiments, one single histone octamer bound to DNAs within 1500 bps flanking a target sequence in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 1500 bps flanking a promoter sequence of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more histone tails of histones bound to DNAs within 1500 bps flanking a target sequence in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1500 bps flanking a target sequence in the epigenetically edited chromosome in a cell are demethylated as compared to the original state of the chromosome or the chromosome in a comparable cell not contacted with the epigenetic editor. In some embodiments, one single histone tail of histones bound to DNAs within 1500 bps flanking a target sequence in the epigenetically edited chromosome in a cell is demethylated as compared to the original state of the chromosome or the chromosome in a comparable cell not contacted with the epigenetic editor. In some embodiments, one single histone octamer bound to DNAs within 1500 bps flanking a target sequence in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 1500 bps flanking a promoter sequence of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more histone tails of histones bound to DNAs within 1500 bps flanking a target sequence in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1500 bps flanking a target sequence in the epigenetically edited chromosome in a cell are acetylated as compared to the original state of the chromosome or the chromosome in a comparable cell not contacted with the epigenetic editor. In some embodiments, one single histone tail of histones bound to DNAs within 1500 bps flanking a target sequence in the epigenetically edited chromosome in a cell is acetylated as compared to the original state of the chromosome or the chromosome in a comparable cell not contacted with the epigenetic editor. In some embodiments, one single histone octamer bound to DNAs within 1500 bps flanking a target sequence in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 1500 bps flanking a promoter sequence of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90 or more histone tails of histones bound to DNAs within 1500 bps flanking a target sequence in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1500 bps flanking a target sequence in the epigenetically edited chromosome in a cell are deacetylated as compared to the original state of the chromosome or the chromosome in a comparable cell not contacted with the epigenetic editor. In some embodiments, one single histone tail of histones bound to DNAs within 1500 bps flanking a target sequence in the epigenetically edited chromosome in a cell is deacetylated as compared to the original state of the chromosome or the chromosome in a comparable cell not contacted with the epigenetic editor. In some embodiments, one single histone octamer bound to DNAs within 1500 bps flanking a target sequence in the epigenetically edited chromosome in a cell is deacetylated as compared to the original state of the chromosome or the chromosome in a comparable cell not contacted with the epigenetic editor.

In some embodiments, all CpG dinucleotides within 1000 bps flanking a target sequence in the epigenetically edited chromosome in a cell are methylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500 or more CpG dinucleotides within 1000 bps flanking a target sequence in the epigenetically edited chromosome in a cell are methylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 1000 bps flanking a target sequence in the epigenetically edited chromosome in a cell are methylated as compared to the original state of the gene or the gene in a comparable cell not contacted with the epigenetic editor. In some embodiments, one single CpG dinucleotide within 1000 bps flanking a target sequence in the epigenetically edited chromosome in a cell is methylated as compared to the original state of the gene or the gene in a comparable cell not contacted with the epigenetic editor.

In some embodiments, all CpG dinucleotides within 1000 bps flanking a target sequence in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500 or more CpG dinucleotides within 1000 bps flanking a target sequence in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 1000 bps flanking a target sequence in the epigenetically edited chromosome in a cell are demethylated as compared to the original state of the gene or the gene in a comparable cell not contacted with the epigenetic editor. In some embodiments, one single CpG dinucleotide within 1000 bps flanking a target sequence in the epigenetically edited chromosome in a cell is demethylated as compared to the original state of the gene or the gene in a comparable cell not contacted with the epigenetic editor.

In some embodiments, all histone tails of histones bound to DNA nucleotides within 1000 bps flanking a promoter sequence of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60 or more histone tails of histones bound to DNAs within 1000 bps flanking a target sequence in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1000 bps flanking a target sequence in the epigenetically edited chromosome in a cell are methylated as compared to the original state of the chromosome or the chromosome in a comparable cell not contacted with the epigenetic editor. In some embodiments, one single histone tail of histones bound to DNAs within 1000 bps flanking a target sequence in the epigenetically edited chromosome in a cell is methylated as compared to the original state of the chromosome or the chromosome in a comparable cell not contacted with the epigenetic editor. In some embodiments, one single histone octamer bound to DNAs within 1000 bps flanking a target sequence in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 1000 bps flanking a promoter sequence of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60 or more histone tails of histones bound to DNAs within 1000 bps flanking a target sequence in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1000 bps flanking a target sequence in the epigenetically edited chromosome in a cell are demethylated as compared to the original state of the chromosome or the chromosome in a comparable cell not contacted with the epigenetic editor. In some embodiments, one single histone tail of histones bound to DNAs within 1000 bps flanking a target sequence in the epigenetically edited chromosome in a cell is demethylated as compared to the original state of the chromosome or the chromosome in a comparable cell not contacted with the epigenetic editor. In some embodiments, one single histone octamer bound to DNAs within 1000 bps flanking a target sequence in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 1000 bps flanking a promoter sequence of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60 or more histone tails of histones bound to DNAs within 1000 bps flanking a target sequence in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1000 bps flanking a target sequence in the epigenetically edited chromosome in a cell are acetylated as compared to the original state of the chromosome or the chromosome in a comparable cell not contacted with the epigenetic editor. In some embodiments, one single histone tail of histones bound to DNAs within 1000 bps flanking a target sequence in the epigenetically edited chromosome in a cell is acetylated as compared to the original state of the chromosome or the chromosome in a comparable cell not contacted with the epigenetic editor. In some embodiments, one single histone octamer bound to DNAs within 1000 bps flanking a target sequence in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 1000 bps flanking a promoter sequence of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60 or more histone tails of histones bound to DNAs within 1000 bps flanking a target sequence in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 1000 bps flanking a target sequence in the epigenetically edited chromosome in a cell are deacetylated as compared to the original state of the chromosome or the chromosome in a comparable cell not contacted with the epigenetic editor. In some embodiments, one single histone tail of histones bound to DNAs within 1000 bps flanking a target sequence in the epigenetically edited chromosome in a cell is deacetylated as compared to the original state of the chromosome or the chromosome in a comparable cell not contacted with the epigenetic editor. In some embodiments, one single histone octamer bound to DNAs within 1000 bps flanking a target sequence in the epigenetically edited chromosome in a cell is deacetylated as compared to the original state of the chromosome or the chromosome in a comparable cell not contacted with the epigenetic editor.

In some embodiments, all CpG dinucleotides within 500 bps flanking a promoter sequence of a gene in the epigenetically edited chromosome in a cell are methylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200 or more CpG dinucleotides within 500 bps flanking a target sequence in the epigenetically edited chromosome in a cell are methylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 500 bps flanking a target sequence in the epigenetically edited chromosome in a cell are methylated as compared to the original state of the gene or the gene in a comparable cell not contacted with the epigenetic editor. In some embodiments, one single CpG dinucleotide within 500 bps flanking a target sequence in the epigenetically edited chromosome in a cell is methylated as compared to the original state of the gene or the gene in a comparable cell not contacted with the epigenetic editor.

In some embodiments, all CpG dinucleotides within 500 bps flanking a target sequence in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200 or more CpG dinucleotides within 500 bps flanking a target sequence in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 500 bps flanking a target sequence in the epigenetically edited chromosome in a cell are demethylated as compared to the original state of the gene or the gene in a comparable cell not contacted with the epigenetic editor. In some embodiments, one single CpG dinucleotide within 500 bps flanking a target sequence in the epigenetically edited chromosome in a cell is demethylated as compared to the original state of the gene or the gene in a comparable cell not contacted with the epigenetic editor.

In some embodiments, all histone tails of histones bound to DNA nucleotides within 500 bps flanking a promoter sequence of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30 or more histone tails of histones bound to DNAs within 500 bps flanking a target sequence in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 500 bps flanking a target sequence in the epigenetically edited chromosome in a cell are methylated as compared to the original state of the chromosome or the chromosome in a comparable cell not contacted with the epigenetic editor. In some embodiments, one single histone tail of histones bound to DNAs within 500 bps flanking a target sequence in the epigenetically edited chromosome in a cell is methylated as compared to the original state of the chromosome or the chromosome in a comparable cell not contacted with the epigenetic editor. In some embodiments, one single histone octamer bound to DNAs within 500 bps flanking a target sequence in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 500 bps flanking a promoter sequence of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30 or more histone tails of histones bound to DNAs within 500 bps flanking a target sequence in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 500 bps flanking a target sequence in the epigenetically edited chromosome in a cell are demethylated as compared to the original state of the chromosome or the chromosome in a comparable cell not contacted with the epigenetic editor. In some embodiments, one single histone tail of histones bound to DNAs within 500 bps flanking a target sequence in the epigenetically edited chromosome in a cell is demethylated as compared to the original state of the chromosome or the chromosome in a comparable cell not contacted with the epigenetic editor. In some embodiments, one single histone octamer bound to DNAs within 500 bps flanking a target sequence in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 500 bps flanking a promoter sequence of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30 or more histone tails of histones bound to DNAs within 500 bps flanking a target sequence in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 500 bps flanking a target sequence in the epigenetically edited chromosome in a cell are acetylated as compared to the original state of the chromosome or the chromosome in a comparable cell not contacted with the epigenetic editor. In some embodiments, one single histone tail of histones bound to DNAs within 500 bps flanking a target sequence in the epigenetically edited chromosome in a cell is acetylated as compared to the original state of the chromosome or the chromosome in a comparable cell not contacted with the epigenetic editor. In some embodiments, one single histone octamer bound to DNAs within 500 bps flanking a target sequence in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 500 bps flanking a promoter sequence of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30 or more histone tails of histones bound to DNAs within 500 bps flanking a target sequence in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 500 bps flanking a target sequence in the epigenetically edited chromosome in a cell are deacetylated as compared to the original state of the chromosome or the chromosome in a comparable cell not contacted with the epigenetic editor. In some embodiments, one single histone tail of histones bound to DNAs within 500 bps flanking a target sequence in the epigenetically edited chromosome in a cell is deacetylated as compared to the original state of the chromosome or the chromosome in a comparable cell not contacted with the epigenetic editor. In some embodiments, one single histone octamer bound to DNAs within 500 bps flanking a target sequence in the epigenetically edited chromosome in a cell is deacetylated as compared to the original state of the chromosome or the chromosome in a comparable cell not contacted with the epigenetic editor.

In some embodiments, all CpG dinucleotides within 200 bps flanking a target sequence in the epigenetically edited chromosome in a cell are demethylated as compared to the original state of the gene or the gene in a comparable cell not contacted with the epigenetic editor. In some embodiments, at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90 or more CpG dinucleotides within 200 bps flanking a target sequence in the epigenetically edited chromosome in a cell are demethylated as compared to the original state of the gene or the gene in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of CpG dinucleotides within 200 bps flanking a target sequence in the epigenetically edited chromosome in a cell are demethylated as compared to the original state of the gene or the gene in a comparable cell not contacted with the epigenetic editor. In some embodiments, one single CpG dinucleotide within 200 bps flanking a target sequence in the epigenetically edited chromosome in a cell is demethylated as compared to the original state of the gene or the gene in a comparable cell not contacted with the epigenetic editor.

In some embodiments, all histone tails of histones bound to DNA nucleotides within 200 bps flanking a promoter sequence of a target gene in the epigenetically edited chromosome in a cell are methylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20 or more histone tails of histones bound to DNAs within 200 bps flanking a target sequence in the epigenetically edited chromosome in a cell are methylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 200 bps flanking a target sequence in the epigenetically edited chromosome in a cell are methylated as compared to the original state of the chromosome or the chromosome in a comparable cell not contacted with the epigenetic editor. In some embodiments, one single histone tail of histones bound to DNAs within 200 bps flanking a target sequence in the epigenetically edited chromosome in a cell is methylated as compared to the original state of the chromosome or the chromosome in a comparable cell not contacted with the epigenetic editor. In some embodiments, one single histone octamer bound to DNAs within 200 bps flanking a target sequence in the epigenetically edited chromosome in a cell is methylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 200 bps flanking a promoter sequence of a target gene in the epigenetically edited chromosome in a cell are demethylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20 or more histone tails of histones bound to DNAs within 200 bps flanking a target sequence in the epigenetically edited chromosome in a cell are demethylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 200 bps flanking a target sequence in the epigenetically edited chromosome in a cell are demethylated as compared to the original state of the chromosome or the chromosome in a comparable cell not contacted with the epigenetic editor. In some embodiments, one single histone tail of histones bound to DNAs within 200 bps flanking a target sequence in the epigenetically edited chromosome in a cell is demethylated as compared to the original state of the chromosome or the chromosome in a comparable cell not contacted with the epigenetic editor. In some embodiments, one single histone octamer bound to DNAs within 200 bps flanking a target sequence in the epigenetically edited chromosome in a cell is demethylated as compared to the original state of the chromosome or the chromosome in a comparable cell not contacted with the epigenetic editor. In some embodiments, the histone is histone H3 and methylation is at Lysine 9, marking the target gene in the epigenetically edited chromosome for repressed expression. In some embodiments, the histone is histone H3 and methylation is at Lysine 4, marking the target gene in the epigenetically edited chromosome for increased expression.

In some embodiments, all histone tails of histones bound to DNA nucleotides within 200 bps flanking a promoter sequence of a target gene in the epigenetically edited chromosome in a cell are acetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20 or more histone tails of histones bound to DNAs within 200 bps flanking a target sequence in the epigenetically edited chromosome in a cell are acetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 200 bps flanking a target sequence in the epigenetically edited chromosome in a cell are acetylated as compared to the original state of the chromosome or the chromosome in a comparable cell not contacted with the epigenetic editor. In some embodiments, one single histone tail of histones bound to DNAs within 200 bps flanking a target sequence in the epigenetically edited chromosome in a cell is acetylated as compared to the original state of the chromosome or the chromosome in a comparable cell not contacted with the epigenetic editor. In some embodiments, one single histone octamer bound to DNAs within 200 bps flanking a target sequence in the epigenetically edited chromosome in a cell is acetylated as compared to the original state of the chromosome or the chromosome in a comparable cell not contacted with the epigenetic editor.

In some embodiments, all histone tails of histones bound to DNA nucleotides within 200 bps flanking a promoter sequence of a target gene in the epigenetically edited chromosome in a cell are deacetylated as compared to the original state of the chromosome or the chromosome in a comparable cell not contacted with the epigenetic editor. In some embodiments, at least 1, 2, 3, 4, 5, 6, 7, 8, 9, 10, 11, 12, 13, 14, 15, 16, 17, 18, 19, 20 or more histone tails of histones bound to DNAs within 200 bps flanking a target sequence in the epigenetically edited chromosome in a cell are deacetylated as compared to the original state of the chromosome or the chromosome in a comparable cell not contacted with the epigenetic editor. In some embodiments, at least 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% of histone tails of histones bound to DNAs within 200 bps flanking a target sequence in the epigenetically edited chromosome in a cell are deacetylated as compared to the original state of the chromosome or the chromosome in a comparable cell not contacted with the epigenetic editor. In some embodiments, one single histone tail of histones bound to DNAs within 200 bps flanking a target sequence in the epigenetically edited chromosome in a cell is deacetylated as compared to the original state of the chromosome or the chromosome in a comparable cell not contacted with the epigenetic editor. In some embodiments, one single histone octamer bound to DNAs within 200 bps flanking a target sequence in the epigenetically edited chromosome in a cell is deacetylated as compared to the original state of the chromosome or the chromosome in a comparable cell not contacted with the epigenetic editor.

In some embodiments, the effector domain comprises a histone methyltransferase domain. For example, repression (or silencing) may result from repressive chromatin markers, methylation of DNA, methylation of histone residues (e.g., H3K9, H3K27), or deacetylation of histone residues.) on chromatin containing a target nucleic acid sequence. Without intending to be bound by any theory, the method can be used to change epigenetic state by, for example, closing chromatin via methylation or introducing repressive chromatin markers on chromatin containing the target nuclei acid sequence (e.g., gene).

Specific epigenetic imprints direct gene transcription or gene silencing. For example, DNA methylation, histone modification, repressor proteins binding to silencer regions, and other transcriptional activities alter gene expression without changing the underlying DNA sequence. Thus, the transcriptional regulation allows for expression of specific genes in a particular manner, while repressing other genes. In certain instances, cell fate or function can be controlled, either for initial differentiation (e.g., during the organism's development) or to reprogram a cell or cell type (e.g., during disease such as cancer, chronic inflammation, auto-immune disease, illnesses related to various microbiomes of an organism, etc.). Histone modifications play a structural and biochemical role in gene transcription, in one avenue by formation or disruption of the nucleosome structure that binds to the histone and prevents gene transcription. Histones are basic proteins that are commonly found in the nucleus of eukaryotic cells, ranging from multicellular organisms including humans to unicellular organisms represented by fungi (mold and yeast) and ionically bind to genomic DNA. Histones usually consist of five components (H1, H2A, H2B, H3 and H4) and are highly similar across biological species. In the case of histone H4, for example, budding yeast histone H4 (full-length 102 amino acid sequence) and human histone H4 (full-length 102 amino acid sequence) are identical in 92% of the amino acid sequences and differ only in 8 residues. Among the natural proteins assumed to be present in several tens of thousands of organisms, histones are known to be proteins most highly preserved among eukaryotic species. Genomic DNA is folded with histones by ordered binding, and a complex of the both forms a basic structural unit called a nucleosome. In addition, aggregation of the nucleosomes forms a chromosomal chromatin structure. Histones are subject to modifications, such as acetylation, methylation, phosphorylation, ubiquitination, SUMOylation and the like, at their N-terminal ends called histone tails, and maintain or specifically convert the chromatin structure, thereby controlling responses such as gene expression, DNA replication, DNA repair and the like, which occur on chromosomal DNA. Post-translational modification of histones is an epigenetic regulatory mechanism, and is considered essential for the genetic regulation of eukaryotic cells. Recent studies have revealed that chromatin remodeling factors such as SWI/SNF, RSC, NURF, NRD and the like, which encourage DNA access to transcription factors by modifying the nucleosome structure, histone acetyltransferases (HATs) that regulate the acetylation state of histones, and histone deacetylases (HDACs), act as important regulators. DNA methylation occurs primarily at CpG sites (shorthand for “C-phosphate-G-” or “cytosine-phosphate-guanine”). Highly methylated areas of DNA tend to be less transcriptionally active than lesser methylated sites. Many mammalian genes have promoter regions near or including CpG islands (regions with a high frequency of CpG sites).

In particular, the unstructured N-termini of histones may be modified by at least one of acetylation, methylation, ubiquitylation, phosphorylation, sumoylation, ribosylation, citrullination O-GlcNAcylation, or crotonylation. For example, acetylation of K14 and K9 lysines of histone H3 by histone acetyltransferase enzymes may be linked to transcriptional competence in humans. Lysine acetylation may directly or indirectly create binding sites for chromatin-modifying enzymes that regulate transcriptional activation. For example, histone acetyltransferases (HATs) utilize acetyl-CoA as a cofactor and catalyze the transfer of an acetyl group to the epsilon amino group of the lysine side chains. This neutralizes the lysine's positive charge and weakens the interactions between histones and DNA, thus opening the chromosomes for transcription factors to bind and initiate transcription. Likewise, histone methylation of lysine 9 of histone H3 may be associated with heterochromatin, or transcriptionally silent chromatin. Particular DNA methylation patterns may be established and modified by at least one or more, two or more, three or more, four or more, or five or more independent DNA methyltransferases, including DNMT1, DNMT3A. and DNMT3B.

In some embodiments, the effector domain comprises a histone methyltransferase domain. In some embodiments, the effector domain comprises a DOT1L domain, a SET domain, a SUV39H1 domain, a G9a/EHMT2 protein domain, a EZH1 domain, a EZH2 domain, a SETDB1 domain, or any combination thereof. In some embodiments, the effector domain comprises a histone-lysine-N-methyltransferase SETDB1 domain.

In some embodiments, the effector domain comprises a DNA methyltransferase domain or a Histone methyltransferase domain. DNA methyltransferase domains may mediate methylation at DNA nucleotides, for example at any of an A, T, G or C nucleotide. In some embodiments, the methylated nucleotide is a N6-methyladenosine (m6A). In some embodiments, the methylated nucleotide is a 5-methylcytosine (5mC). In some embodiments, the methylation is at a CG (or CpG) dinucleotide sequence. In some embodiments, the methylation is at a CHG or CHH sequence, where H is any one of A, T, or C.

In some embodiments, the effector domain comprises a DNA methyltransferase DNMT domain that catalyzes transfer of a methyl group to cytosine, thereby repressing expression of the target gene through the recruitment of repressive regulatory proteins. In some embodiments, the effector domain comprises a DNA methyltransferase (DNMT) family protein domain. In some embodiments, the effector domain comprises a DNMT1 domain. In some embodiments, the effector domain comprises a TRDMT1 domain. In some embodiments, the effector domain comprises a DNMT3 domain. In some embodiments, the effector domain comprises a DNMT3A domain. In some embodiments, the effector domain comprises a DNMT3B domain. In some embodiments, the effector domain comprises a DNMT3C domain. In some embodiments, the effector domain comprises a DNMT3L domain. In some embodiments, the effector domain comprises a fusion of DNMT3A-DNMT3L domain.

Exemplary methyltransferase that may be part of an epigenetic effector domain are provided in Table 1 below.

TABLE 1
Exemplary methyltransferase sequences that
may be used in epigenetic effector domains

| Protein Name | Species | Target | Protein Sequence |
|---|---|---|---|
| DNMT1 | Human | 5mC | SEQ ID NO.: 32 |
| DNMT3A | Human | 5mC | SEQ ID NO.: 33 |
| DNMT3B | Human | 5mC | SEQ ID NO.: 35 |
| DNMT3C | Mouse | 5mC | SEQ ID NO.: 36 |
| DNMT3L | Human | 5mC | SEQ ID NO.: 37 |
| DNMT3L | Mouse | 5mC | SEQ ID NO.: 39 |
| TRDMT1 | Human | tRNA 5mC | SEQ ID NO.: 41 |
| (DNMT2) | | | |
| M. MpeI | Mycoplasma penetrans | 5mC | SEQ ID NO.: 42 |
| M. SssI | Spiroplasma monobiae | 5mC | SEQ ID NO.: 43 |
| M. HpaII | Haemophilus parainfluenzae | 5mC (CCGG) | SEQ ID NO.: 44 |
| M. AluI | Arthrobacter luteus | 5mC (AGCT) | SEQ ID NO.: 45 |
| M. HaeIII | Haemophilus aegyptius | 5mC (GGCC) | SEQ ID NO.: 46 |
| M. HhaI | Haemophilus haemolyticus | 5mC (GCGC) | SEQ ID NO.: 47 |
| M. MspI | Moraxella | 5mC (CCGG) | SEQ ID NO.: 48 |
| Masc1 | Ascobolus | 5mC | SEQ ID NO.: 49 |
| MET1 | Arabidopsis | 5mC | SEQ ID NO.: 50 |
| Masc2 | Ascobolus | 5mC | SEQ ID NO.: 51 |
| Dim-2 | Neurospora | 5mC | SEQ ID NO.: 52 |
| dDnmt2 | Drosophila | 5mC | SEQ ID NO.: 53 |
| Pmt1 | S. Pombe | 5mC | SEQ ID NO.: 54 |
| DRM1 | Arabidopsis | 5mC | SEQ ID NO.: 55 |
| DRM2 | Arabidopsis | 5mC | SEQ ID NO.: 56 |
| CMT1 | Arabidopsis | 5mC | SEQ ID NO.: 57 |
| CMT2 | Arabidopsis | 5mC | SEQ ID NO.: 58 |
| CMT3 | Arabidopsis | 5mC | SEQ ID NO.: 59 |
| Rid | Neurospora | 5mC | SEQ ID NO.: 60 |
| hsdM gene | bacteria (E.coli, strain 12) | m6A | SEQ ID NO.: 61 |
| hsdS gene | bacteria (E.coli, strain 12) | m6A | SEQ ID NO.: 62 |
| M. TaqI | bacteria; Thermus aquaticus | m6A | SEQ ID NO.: 63 |
| M. EcoDam | E. coli | m6A | SEQ ID NO.: 64 |
| M. CcrMI | Caulobacter crescentus | m6A | SEQ ID NO.: 65 |
| CamA | Clostridioides difficile | m6A | SEQ ID NO.: 66 |

In some embodiments, the effector domain recruits one or more protein domains that repress expression of the target gene. In some embodiments, the effector domain interacts with a scaffold protein domain that recruits one or more protein domains that repress expression of the target gene. For example, the effector domain may recruit or interact with a scaffold protein domain that recruits a PRMT protein, a HDAC protein, a SETDB1 protein, or a NuRD protein domain. In some embodiments, the effector domain comprises a Krippel associated box (KRAB) repression domain; a Repressor Element Silencing Transcription Factor (REST) repression domain, KRAB-associated protein 1 (KAP1) domain, a MAD domain, a FKHR (forkhead in rhabdosarcoma gene) repressor domain, aEGR-1 (early growth response gene product-1) repressor domain, a ets2 repressor factor repressor domain (ERD), a MAD smSIN3 interaction domain (SID), a WRPW motif (SEQ ID NO: 1162) of the hairy-related basic helix-loop-helix (bHLH) repressor proteins; an HP1 alpha chromo-shadow repression domain, or any combination thereof. In some embodiments, the effector domain comprises a KRAB domain. In some embodiments, the effector domain comprises a Tripartite motif containing 28 (TRIM28, TIF1-beta, or KAP1) protein.

In some embodiments, an effector domain comprises a protein domain that represses expression of the target gene. For example, the effector domain may comprise a functional domain derived from a zinc finger repressor protein. In some embodiments, the effector domain comprises a functional repression domain derived from a KOX1/ZNF10 domain, a KOX8/ZNF708 domain, a ZNF43 domain, a ZNF184 domain, a ZNF91 KRAB domain, a HPF4 domain, a HTF10 domain or a HTF34 domain or any combination thereof. In some embodiments, the effector domain comprises a functional repression domain derived from a ZIM3 protein domain, a ZNF436 domain, a ZNF257 domain, a ZNF675 domain, a ZNF490 domain, a ZNF320 domain, a ZNF331 domain, a ZNF816 domain, a ZNF680 domain, a ZNF41 domain, a ZNF189 domain, a ZNF528 domain, a ZNF543 domain, a ZNF554 domain, a ZNF140 domain, a ZNF610 domain, a ZNF264 domain, a ZNF350 domain, a ZNF8 domain, a ZNF582 domain, a ZNF30 domain, a ZNF324 domain, a ZNF98 domain, a ZNF669 domain, a ZNF677 domain, a ZNF596 domain, a ZNF214 domain, a ZNF37A domain, a ZNF34 domain, a ZNF250 domain, a ZNF547 domain, a ZNF273 domain, a ZNF354A domain, a ZFP82 domain, a ZNF224 domain, a ZNF33A domain, a ZNF45 domain, a ZNF175 domain, a ZNF595 domain, a ZNF184 domain, a ZNF419 domain, a ZFP28-1 domain, a ZFP28-2 domain, a ZNF18 domain, a ZNF213 domain, a ZNF394 domain, a ZFP1 domain, a ZFP14 domain, a ZNF416 domain, a ZNF557 domain, a ZNF566 domain, a ZNF729 domain, a ZIM2 domain, a ZNF254 domain, a ZNF764 domain, a ZNF785 domain or any combination thereof. In some embodiments, the domain is a ZIM3 domain, a ZNF554 domain, a ZNF264 domain, a ZNF324 domain, a ZNF354A domain, a ZNF189 domain, a ZNF543 domain, a ZFP82 domain, a ZNF669 domain, or a ZNF582 domain or any combination thereof. In some embodiments, the domain is a ZIM3 domain, a ZNF554 domain, a ZNF264 domain, a ZNF324 domain, or a ZNF354A domain or any combination thereof. In some embodiments, the domain is a ZIM3 domain.

In some embodiments, an effector domain can be an alternate KRAB domain (e.g.,). Alternatively or in addition to, an effector domain can be a non-KRAB domain (e.g.)

In some embodiments, the protein fusion construct can have 1 effector domain, 2 effector domains, 3 effector domains, 4 effector domains, 5 effector domains, 6 effector domains, 7 effector domains, 8 effector domains, 9 effector domains, or 10 effector domains.

Sequences of exemplary functional domains that may reduce or silence target gene expression are provided in Table 2 below. Further examples of repressors and repressor domains can be found in PCT/US2021/030643 and Tycko et al. (Tycko J, DelRosso N, Hess G T, Aradhana, Banerjee A, Mukund A, Van M V, Ego B K, Yao D, Spees K, Suzuki P, Marinov G K, Kundaje A, Bassik M C, Bintu L. High-Throughput Discovery and Characterization of Human Transcriptional Effectors. Cell. 2020 Dec. 23; 183(7):2020-2035.e16. doi: 10.1016/j.cell.2020.11.024. Epub 2020 Dec. 15. PMID: 33326746; PMCID: PMC8178797.), which are incorporated here by reference to it entirety.

TABLE 2
Exemplary effector domains that may
reduce or silence gene expression

| Protein | Protein Sequence |
|---|---|
| ZIM3 | SEQ ID NO.: 67 |
| ZNF436 | SEQ ID NO.: 68 |
| ZNF257 | SEQ ID NO.: 69 |
| ZNF675 | SEQ ID NO.: 70 |
| ZNF490 | SEQ ID NO.: 71 |
| ZNF320 | SEQ ID NO.: 72 |
| ZNF331 | SEQ ID NO.: 73 |
| ZNF816 | SEQ ID NO.: 74 |
| ZNF680 | SEQ ID NO.: 75 |
| ZNF41 | SEQ ID NO.: 76 |
| ZNF189 | SEQ ID NO.: 77 |
| ZNF528 | SEQ ID NO.: 78 |
| ZNF543 | SEQ ID NO.: 79 |
| ZNF554 | SEQ ID NO.: 80 |
| ZNF140 | SEQ ID NO.: 81 |
| ZNF610 | SEQ ID NO.: 82 |
| ZNF264 | SEQ ID NO.: 83 |
| ZNF350 | SEQ ID NO.: 84 |
| ZNF8 | SEQ ID NO.: 85 |
| ZNF582 | SEQ ID NO.: 86 |
| ZNF30 | SEQ ID NO.: 87 |
| ZNF324 | SEQ ID NO.: 88 |
| ZNF98 | SEQ ID NO.: 89 |
| ZNF669 | SEQ ID NO.: 90 |
| ZNF677 | SEQ ID NO.: 91 |
| ZNF596 | SEQ ID NO.: 92 |
| ZNF214 | SEQ ID NO.: 93 |
| ZNF37A | SEQ ID NO.: 94 |
| ZNF34 | SEQ ID NO.: 95 |
| ZNF250 | SEQ ID NO.: 96 |
| ZNF547 | SEQ ID NO.: 97 |
| ZNF273 | SEQ ID NO.: 98 |
| ZNF354A | SEQ ID NO.: 99 |
| ZFP82 | SEQ ID NO.: 100 |
| ZNF224 | SEQ ID NO.: 101 |
| ZNF33A | SEQ ID NO.: 102 |
| ZNF45 | SEQ ID NO.: 103 |
| ZNF175 | SEQ ID NO.: 104 |
| ZNF595 | SEQ ID NO.: 105 |
| ZNF184 | SEQ ID NO.: 106 |
| ZNF419 | SEQ ID NO.: 107 |
| ZFP28-1 | SEQ ID NO.: 108 |
| ZFP28-2 | SEQ ID NO.: 109 |
| ZNF18 | SEQ ID NO.: 110 |
| ZNF213 | SEQ ID NO.: 111 |
| ZNF394 | SEQ ID NO.: 112 |
| ZFP1 | SEQ ID NO.: 113 |
| ZFP14 | SEQ ID NO.: 114 |
| ZNF416 | SEQ ID NO.: 115 |
| ZNF557 | SEQ ID NO.: 116 |
| ZNF566 | SEQ ID NO.: 117 |
| ZNF729 | SEQ ID NO.: 118 |
| ZIM2 | SEQ ID NO.: 119 |
| ZNF254 | SEQ ID NO.: 120 |
| ZNF764 | SEQ ID NO.: 121 |
| ZNF785 | SEQ ID NO.: 122 |
| ZNF10 (KOX1) | SEQ ID NO.: 123 |
| CBX5 (chromoshadow domain) | SEQ ID NO.: 124 |
| RYBP (YAF2_RYBP component of PRC1) | SEQ ID NO.: 125 |
| YAF2 (YAF2_RYBP component of PRC1) | SEQ ID NO.: 126 |
| MGA (component of PRC1.6) | SEQ ID NO.: 127 |
| CBX1 (chromoshadow) | SEQ ID NO.: 128 |
| SCMH1 (SAM_1/SPM) | SEQ ID NO.: 129 |
| MPP8 (Chromodomain) | SEQ ID NO.: 130 |
| SUMO3 (Rad60-SLD) | SEQ ID NO.: 131 |
| HERC2 (Cyt-b5) | SEQ ID NO.: 132 |
| BIN1 (SH3_9) | SEQ ID NO.: 133 |
| PCGF2 (RING finger protein domain) | SEQ ID NO.: 134 |
| TOX (HMG box) | SEQ ID NO.: 135 |
| FOXA1 (HNF3A C-terminal domain) | SEQ ID NO.: 136 |
| FOXA2 (HNF3B C-terminal domain) | SEQ ID NO.: 137 |
| IRF2BP1 (IRF-2BP1_2 N-terminal domain) | SEQ ID NO.: 138 |
| IRF2BP2 (IRF-2BP1_2 N-terminal domain) | SEQ ID NO.: 139 |
| IRF2BPL IRF-2BP1_2 N-terminal domain | SEQ ID NO.: 140 |
| HOXA13 (homeodomain) | SEQ ID NO.: 141 |
| HOXB13 (homeodomain) | SEQ ID NO.: 142 |
| HOXC13 (homeodomain) | SEQ ID NO.: 143 |
| HOXA11 (homeodomain) | SEQ ID NO.: 144 |
| HOXC11 (homeodomain) | SEQ ID NO.: 145 |
| HOXC10 (homeodomain) | SEQ ID NO.: 146 |
| HOXA10 (homeodomain) | SEQ ID NO.: 147 |
| HOXB9 (homeodomain) | SEQ ID NO.: 148 |
| HOXA9 (homeodomain) | SEQ ID NO.: 149 |

Sequences of additional exemplary functional domains that may reduce or silence target gene expression are provided in Table 3 below.

TABLE 3
Exemplary effector domains that may
reduce or silence gene expression

| Gene name | Extended Domain sequence |
|---|---|
| ZFP28_HUMAN | SEQ ID NO.: 150 |
| ZN334_HUMAN | SEQ ID NO.: 151 |
| ZN568_HUMAN | SEQ ID NO.: 152 |
| ZN37A_HUMAN | SEQ ID NO.: 153 |
| ZN181_HUMAN | SEQ ID NO.: 154 |
| ZN510_HUMAN | SEQ ID NO.: 155 |
| ZN862_HUMAN | SEQ ID NO.: 156 |
| ZN140_HUMAN | SEQ ID NO.: 157 |
| ZN208_HUMAN | SEQ ID NO.: 158 |
| ZN248_HUMAN | SEQ ID NO.: 159 |
| ZN571_HUMAN | SEQ ID NO.: 160 |
| ZN699_HUMAN | SEQ ID NO.: 161 |
| ZN726_HUMAN | SEQ ID NO.: 162 |
| ZIK1_HUMAN | SEQ ID NO.: 163 |
| ZNF2_HUMAN | SEQ ID NO.: 164 |
| Z705F_HUMAN | SEQ ID NO.: 165 |
| ZNF14_HUMAN | SEQ ID NO.: 166 |
| ZN471_HUMAN | SEQ ID NO.: 167 |
| ZN624_HUMAN | SEQ ID NO.: 168 |
| ZNF84_HUMAN | SEQ ID NO.: 169 |
| ZNF7_HUMAN | SEQ ID NO.: 170 |
| ZN891_HUMAN | SEQ ID NO.: 171 |
| ZN337_HUMAN | SEQ ID NO.: 172 |
| Z705G_HUMAN | SEQ ID NO.: 173 |
| ZN529_HUMAN | SEQ ID NO.: 174 |
| ZN729_HUMAN | SEQ ID NO.: 175 |
| ZN419_HUMAN | SEQ ID NO.: 176 |
| Z705A_HUMAN | SEQ ID NO.: 177 |
| ZNF45_HUMAN | SEQ ID NO.: 178 |
| ZN302_HUMAN | SEQ ID NO.: 179 |
| ZN486_HUMAN | SEQ ID NO.: 180 |
| ZN621_HUMAN | SEQ ID NO.: 181 |
| ZN688_HUMAN | SEQ ID NO.: 182 |
| ZN33A_HUMAN | SEQ ID NO.: 183 |
| ZN554_HUMAN | SEQ ID NO.: 184 |
| ZN878_HUMAN | SEQ ID NO.: 185 |
| ZN772_HUMAN | SEQ ID NO.: 186 |
| ZN224_HUMAN | SEQ ID NO.: 187 |
| ZN184_HUMAN | SEQ ID NO.: 188 |
| ZN544_HUMAN | SEQ ID NO.: 189 |
| ZNF57_HUMAN | SEQ ID NO.: 190 |
| ZN283_HUMAN | SEQ ID NO.: 191 |
| ZN549_HUMAN | SEQ ID NO.: 192 |
| ZN211_HUMAN | SEQ ID NO.: 193 |
| ZN615_HUMAN | SEQ ID NO.: 194 |
| ZN253_HUMAN | SEQ ID NO.: 195 |
| ZN226_HUMAN | SEQ ID NO.: 196 |
| ZN730_HUMAN | SEQ ID NO.: 197 |
| Z585A_HUMAN | SEQ ID NO.: 198 |
| ZN732_HUMAN | SEQ ID NO.: 199 |
| ZN681_HUMAN | SEQ ID NO.: 200 |
| ZN667_HUMAN | SEQ ID NO.: 201 |
| ZN649_HUMAN | SEQ ID NO.: 202 |
| ZN470_HUMAN | SEQ ID NO.: 203 |
| ZN484_HUMAN | SEQ ID NO.: 204 |
| ZN431_HUMAN | SEQ ID NO.: 205 |
| ZN382_HUMAN | SEQ ID NO.: 206 |
| ZN254_HUMAN | SEQ ID NO.: 207 |
| ZN124_HUMAN | SEQ ID NO.: 208 |
| ZN607_HUMAN | SEQ ID NO.: 209 |
| ZN317_HUMAN | SEQ ID NO.: 210 |
| ZN620_HUMAN | SEQ ID NO.: 211 |
| ZN141_HUMAN | SEQ ID NO.: 212 |
| ZN584_HUMAN | SEQ ID NO.: 213 |
| ZN540_HUMAN | SEQ ID NO.: 214 |
| ZN75D_HUMAN | SEQ ID NO.: 215 |
| ZN555_HUMAN | SEQ ID NO.: 216 |
| ZN658_HUMAN | SEQ ID NO.: 217 |
| ZN684_HUMAN | SEQ ID NO.: 218 |
| RBAK_HUMAN | SEQ ID NO.: 219 |
| ZN829_HUMAN | SEQ ID NO.: 220 |
| ZN582_HUMAN | SEQ ID NO.: 221 |
| ZN112_HUMAN | SEQ ID NO.: 222 |
| ZN716_HUMAN | SEQ ID NO.: 223 |
| HKR1_HUMAN | SEQ ID NO.: 224 |
| ZN350_HUMAN | SEQ ID NO.: 225 |
| ZN480_HUMAN | SEQ ID NO.: 226 |
| ZN416_HUMAN | SEQ ID NO.: 227 |
| ZNF92_HUMAN | SEQ ID NO.: 228 |
| ZN100_HUMAN | SEQ ID NO.: 229 |
| ZN736_HUMAN | SEQ ID NO.: 230 |
| ZNF74_HUMAN | SEQ ID NO.: 231 |
| CBX1_HUMAN | SEQ ID NO.: 232 |
| ZN443_HUMAN | SEQ ID NO.: 233 |
| ZN195_HUMAN | SEQ ID NO.: 234 |
| ZN530_HUMAN | SEQ ID NO.: 235 |
| ZN782_HUMAN | SEQ ID NO.: 236 |
| ZN791_HUMAN | SEQ ID NO.: 237 |
| ZN331_HUMAN | SEQ ID NO.: 238 |
| Z354C_HUMAN | SEQ ID NO.: 239 |
| ZN157_HUMAN | SEQ ID NO.: 240 |
| ZN727_HUMAN | SEQ ID NO.: 241 |
| ZN550_HUMAN | SEQ ID NO.: 242 |
| ZN793_HUMAN | SEQ ID NO.: 243 |
| ZN235_HUMAN | SEQ ID NO.: 244 |
| ZNF8_HUMAN | SEQ ID NO.: 245 |
| ZN724_HUMAN | SEQ ID NO.: 246 |
| ZN573_HUMAN | SEQ ID NO.: 247 |
| ZN577_HUMAN | SEQ ID NO.: 248 |
| ZN789_HUMAN | SEQ ID NO.: 249 |
| ZN718_HUMAN | SEQ ID NO.: 250 |
| ZN300_HUMAN | SEQ ID NO.: 251 |
| ZN383_HUMAN | SEQ ID NO.: 252 |
| ZN429_HUMAN | SEQ ID NO.: 253 |
| ZN677_HUMAN | SEQ ID NO.: 254 |
| ZN850_HUMAN | SEQ ID NO.: 255 |
| ZN454_HUMAN | SEQ ID NO.: 256 |
| ZN257_HUMAN | SEQ ID NO.: 257 |
| ZN264_HUMAN | SEQ ID NO.: 258 |
| ZFP82_HUMAN | SEQ ID NO.: 259 |
| ZFP14_HUMAN | SEQ ID NO.: 260 |
| ZN485_HUMAN | SEQ ID NO.: 261 |
| ZN737_HUMAN | SEQ ID NO.: 262 |
| ZNF44_HUMAN | SEQ ID NO.: 263 |
| ZN596_HUMAN | SEQ ID NO.: 264 |
| ZN565_HUMAN | SEQ ID NO.: 265 |
| ZN543_HUMAN | SEQ ID NO.: 266 |
| ZFP69_HUMAN | SEQ ID NO.: 267 |
| SUMO1_HUMAN | SEQ ID NO.: 268 |
| ZNF12_HUMAN | SEQ ID NO.: 269 |
| ZN169_HUMAN | SEQ ID NO.: 270 |
| ZN433_HUMAN | SEQ ID NO.: 271 |
| SUMO3_HUMAN | SEQ ID NO.: 272 |
| ZNF98_HUMAN | SEQ ID NO.: 273 |
| ZN175_HUMAN | SEQ ID NO.: 274 |
| ZN347_HUMAN | SEQ ID NO.: 275 |
| ZNF25_HUMAN | SEQ ID NO.: 276 |
| ZN519_HUMAN | SEQ ID NO.: 277 |
| Z585B_HUMAN | SEQ ID NO.: 278 |
| ZIM3_HUMAN | SEQ ID NO.: 279 |
| ZN517_HUMAN | SEQ ID NO.: 280 |
| ZN846_HUMAN | SEQ ID NO.: 281 |
| ZN230_HUMAN | SEQ ID NO.: 282 |
| ZNF66_HUMAN | SEQ ID NO.: 283 |
| ZFP1_HUMAN | SEQ ID NO.: 284 |
| ZN713_HUMAN | SEQ ID NO.: 285 |
| ZN816_HUMAN | SEQ ID NO.: 286 |
| ZN426_HUMAN | SEQ ID NO.: 287 |
| ZN674_HUMAN | SEQ ID NO.: 288 |
| ZN627_HUMAN | SEQ ID NO.: 289 |
| ZNF20_HUMAN | SEQ ID NO.: 290 |
| Z587B_HUMAN | SEQ ID NO.: 291 |
| ZN316_HUMAN | SEQ ID NO.: 292 |
| ZN233_HUMAN | SEQ ID NO.: 293 |
| ZN611_HUMAN | SEQ ID NO.: 294 |
| ZN556_HUMAN | SEQ ID NO.: 295 |
| ZN234_HUMAN | SEQ ID NO.: 296 |
| ZN560_HUMAN | SEQ ID NO.: 297 |
| ZNF77_HUMAN | SEQ ID NO.: 298 |
| ZN682_HUMAN | SEQ ID NO.: 299 |
| ZN614_HUMAN | SEQ ID NO.: 300 |
| ZN785_HUMAN | SEQ ID NO.: 301 |
| ZN445_HUMAN | SEQ ID NO.: 302 |
| ZFP30_HUMAN | SEQ ID NO.: 303 |
| ZN225_HUMAN | SEQ ID NO.: 304 |
| ZN551_HUMAN | SEQ ID NO.: 305 |
| ZN610_HUMAN | SEQ ID NO.: 306 |
| ZN528_HUMAN | SEQ ID NO.: 307 |
| ZN284_HUMAN | SEQ ID NO.: 308 |
| ZN418_HUMAN | SEQ ID NO.: 309 |
| MPP8_HUMAN | SEQ ID NO.: 310 |
| ZN490_HUMAN | SEQ ID NO.: 311 |
| ZN805_HUMAN | SEQ ID NO.: 312 |
| Z780B_HUMAN | SEQ ID NO.: 313 |
| ZN763_HUMAN | SEQ ID NO.: 314 |
| ZN285_HUMAN | SEQ ID NO.: 315 |
| ZNF85_HUMAN | SEQ ID NO.: 316 |
| ZN223_HUMAN | SEQ ID NO.: 317 |
| ZNF90_HUMAN | SEQ ID NO.: 318 |
| ZN557_HUMAN | SEQ ID NO.: 319 |
| ZN425_HUMAN | SEQ ID NO.: 320 |
| ZN229_HUMAN | SEQ ID NO.: 321 |
| ZN606_HUMAN | SEQ ID NO.: 322 |
| ZN155_HUMAN | SEQ ID NO.: 323 |
| ZN222_HUMAN | SEQ ID NO.: 324 |
| ZN442_HUMAN | SEQ ID NO.: 325 |
| ZNF91_HUMAN | SEQ ID NO.: 326 |
| ZN135_HUMAN | SEQ ID NO.: 327 |
| ZN778_HUMAN | SEQ ID NO.: 328 |
| RYBP_HUMAN | SEQ ID NO.: 329 |
| ZN534_HUMAN | SEQ ID NO.: 330 |
| ZN586_HUMAN | SEQ ID NO.: 331 |
| ZN567_HUMAN | SEQ ID NO.: 332 |
| ZN440_HUMAN | SEQ ID NO.: 333 |
| ZN583_HUMAN | SEQ ID NO.: 334 |
| ZN441_HUMAN | SEQ ID NO.: 335 |
| ZNF43_HUMAN | SEQ ID NO.: 336 |
| CBX5_HUMAN | SEQ ID NO.: 337 |
| ZN589_HUMAN | SEQ ID NO.: 338 |
| ZNF10_HUMAN | SEQ ID NO.: 339 |
| ZN563_HUMAN | SEQ ID NO.: 340 |
| ZN561_HUMAN | SEQ ID NO.: 341 |
| ZN136_HUMAN | SEQ ID NO.: 342 |
| ZN630_HUMAN | SEQ ID NO.: 343 |
| ZN527_HUMAN | SEQ ID NO.: 344 |
| ZN333_HUMAN | SEQ ID NO.: 345 |
| Z324B_HUMAN | SEQ ID NO.: 346 |
| ZN786_HUMAN | SEQ ID NO.: 347 |
| ZN709_HUMAN | SEQ ID NO.: 348 |
| ZN792_HUMAN | SEQ ID NO.: 349 |
| ZN599_HUMAN | SEQ ID NO.: 350 |
| ZN613_HUMAN | SEQ ID NO.: 351 |
| ZF69B_HUMAN | SEQ ID NO.: 352 |
| ZN799_HUMAN | SEQ ID NO.: 353 |
| ZN569_HUMAN | SEQ ID NO.: 354 |
| ZN564_HUMAN | SEQ ID NO.: 355 |
| ZN546_HUMAN | SEQ ID NO.: 356 |
| ZFP92_HUMAN | SEQ ID NO.: 357 |
| YAF2_HUMAN | SEQ ID NO.: 358 |
| ZN723_HUMAN | SEQ ID NO.: 359 |
| ZNF34_HUMAN | SEQ ID NO.: 360 |
| ZN439_HUMAN | SEQ ID NO.: 361 |
| ZFP57_HUMAN | SEQ ID NO.: 362 |
| ZNF19_HUMAN | SEQ ID NO.: 363 |
| ZN404_HUMAN | SEQ ID NO.: 364 |
| ZN274_HUMAN | SEQ ID NO.: 365 |
| CBX3_HUMAN | SEQ ID NO.: 366 |
| ZNF30_HUMAN | SEQ ID NO.: 367 |
| ZN250_HUMAN | SEQ ID NO.: 368 |
| ZN570_HUMAN | SEQ ID NO.: 369 |
| ZN675_HUMAN | SEQ ID NO.: 370 |
| ZN695_HUMAN | SEQ ID NO.: 371 |
| ZN548_HUMAN | SEQ ID NO.: 372 |
| ZN132_HUMAN | SEQ ID NO.: 373 |
| ZN738_HUMAN | SEQ ID NO.: 374 |
| ZN420_HUMAN | SEQ ID NO.: 375 |
| ZN626_HUMAN | SEQ ID NO.: 376 |
| ZN559_HUMAN | SEQ ID NO.: 377 |
| ZN460_HUMAN | SEQ ID NO.: 378 |
| ZN268_HUMAN | SEQ ID NO.: 379 |
| ZN304_HUMAN | SEQ ID NO.: 380 |
| ZIM2_HUMAN | SEQ ID NO.: 381 |
| ZN605_HUMAN | SEQ ID NO.: 382 |
| ZN844_HUMAN | SEQ ID NO.: 383 |
| SUMO5_HUMAN | SEQ ID NO.: 384 |
| ZN101_HUMAN | SEQ ID NO.: 385 |
| ZN783_HUMAN | SEQ ID NO.: 386 |
| ZN417_HUMAN | SEQ ID NO.: 387 |
| ZN182_HUMAN | SEQ ID NO.: 388 |
| ZN823_HUMAN | SEQ ID NO.: 389 |
| ZN177_HUMAN | SEQ ID NO.: 390 |
| ZN197_HUMAN | SEQ ID NO.: 391 |
| ZN717_HUMAN | SEQ ID NO.: 392 |
| ZN669_HUMAN | SEQ ID NO.: 393 |
| ZN256_HUMAN | SEQ ID NO.: 394 |
| ZN251_HUMAN | SEQ ID NO.: 395 |
| CBX4_HUMAN | SEQ ID NO.: 396 |
| PCGF2_HUMAN | SEQ ID NO.: 397 |
| CDY2_HUMAN | SEQ ID NO.: 398 |
| CDYL2_HUMAN | SEQ ID NO.: 399 |
| HERC2_HUMAN | SEQ ID NO.: 400 |
| ZN562_HUMAN | SEQ ID NO.: 401 |
| ZN461_HUMAN | SEQ ID NO.: 402 |
| Z324A_HUMAN | SEQ ID NO.: 403 |
| ZN766_HUMAN | SEQ ID NO.: 404 |
| ID2_HUMAN | SEQ ID NO.: 405 |
| TOX_HUMAN | SEQ ID NO.: 406 |
| ZN274_HUMAN | SEQ ID NO.: 407 |
| SCMH1_HUMAN | SEQ ID NO.: 408 |
| ZN214_HUMAN | SEQ ID NO.: 409 |
| CBX7_HUMAN | SEQ ID NO.: 410 |
| ID1_HUMAN | SEQ ID NO.: 411 |
| CREM_HUMAN | SEQ ID NO.: 412 |
| SCX_HUMAN | SEQ ID NO.: 413 |
| ASCL1_HUMAN | SEQ ID NO.: 414 |
| ZN764_HUMAN | SEQ ID NO.: 415 |
| SCML2_HUMAN | SEQ ID NO.: 416 |
| TWST1_HUMAN | SEQ ID NO.: 417 |
| CREB1_HUMAN | SEQ ID NO.: 418 |
| TERF1_HUMAN | SEQ ID NO.: 419 |
| ID3_HUMAN | SEQ ID NO.: 420 |
| CBX8_HUMAN | SEQ ID NO.: 421 |
| CBX4_HUMAN | SEQ ID NO.: 422 |
| GSX1_HUMAN | SEQ ID NO.: 423 |
| NKX22_HUMAN | SEQ ID NO.: 424 |
| ATF1_HUMAN | SEQ ID NO.: 425 |
| TWST2_HUMAN | SEQ ID NO.: 426 |
| ZNF17_HUMAN | SEQ ID NO.: 427 |
| TOX3_HUMAN | SEQ ID NO.: 428 |
| TOX4_HUMAN | SEQ ID NO.: 429 |
| ZMYM3_HUMAN | SEQ ID NO.: 430 |
| I2BP1_HUMAN | SEQ ID NO.: 431 |
| RHXF1_HUMAN | SEQ ID NO.: 432 |
| SSX2_HUMAN | SEQ ID NO.: 433 |
| I2BPL_HUMAN | SEQ ID NO.: 434 |
| ZN680_HUMAN | SEQ ID NO.: 435 |
| CBX1_HUMAN | SEQ ID NO.: 436 |
| TRI68_HUMAN | SEQ ID NO.: 437 |
| HXA13_HUMAN | SEQ ID NO.: 438 |
| PHC3_HUMAN | SEQ ID NO.: 439 |
| TCF24_HUMAN | SEQ ID NO.: 440 |
| CBX3_HUMAN | SEQ ID NO.: 441 |
| HXB13_HUMAN | SEQ ID NO.: 442 |
| HEY1_HUMAN | SEQ ID NO.: 443 |
| PHC2_HUMAN | SEQ ID NO.: 444 |
| ZNF81_HUMAN | SEQ ID NO.: 445 |
| FIGLA_HUMAN | SEQ ID NO.: 446 |
| SAM11_HUMAN | SEQ ID NO.: 447 |
| KMT2B_HUMAN | SEQ ID NO.: 448 |
| HEY2_HUMAN | SEQ ID NO.: 449 |
| JDP2_HUMAN | SEQ ID NO.: 450 |
| HXC13_HUMAN | SEQ ID NO.: 451 |
| ASCL4_HUMAN | SEQ ID NO.: 452 |
| HHEX_HUMAN | SEQ ID NO.: 453 |
| HERC2_HUMAN | SEQ ID NO.: 454 |
| GSX2_HUMAN | SEQ ID NO.: 455 |
| BINI_HUMAN | SEQ ID NO.: 456 |
| ETV7_HUMAN | SEQ ID NO.: 457 |
| ASCL3_HUMAN | SEQ ID NO.: 458 |
| PHC1_HUMAN | SEQ ID NO.: 459 |
| OTP_HUMAN | SEQ ID NO.: 460 |
| I2BP2_HUMAN | SEQ ID NO.: 461 |
| VGLL2_HUMAN | SEQ ID NO.: 462 |
| HXA11_HUMAN | SEQ ID NO.: 463 |
| PDLI4_HUMAN | SEQ ID NO.: 464 |
| ASCL2_HUMAN | SEQ ID NO.: 465 |
| CDX4_HUMAN | SEQ ID NO.: 466 |
| ZN860_HUMAN | SEQ ID NO.: 467 |
| LMBL4_HUMAN | SEQ ID NO.: 468 |
| PDIP3_HUMAN | SEQ ID NO.: 469 |
| NKX25_HUMAN | SEQ ID NO.: 470 |
| CEBPB_HUMAN | SEQ ID NO.: 471 |
| ISL1_HUMAN | SEQ ID NO.: 472 |
| CDX2_HUMAN | SEQ ID NO.: 473 |
| PROP1_HUMAN | SEQ ID NO.: 474 |
| SIN3B_HUMAN | SEQ ID NO.: 475 |
| SMBT1_HUMAN | SEQ ID NO.: 476 |
| HXC11_HUMAN | SEQ ID NO.: 477 |
| HXC10_HUMAN | SEQ ID NO.: 478 |
| PRS6A_HUMAN | SEQ ID NO.: 479 |
| VSX1_HUMAN | SEQ ID NO.: 480 |
| NKX23_HUMAN | SEQ ID NO.: 481 |
| MTG16_HUMAN | SEQ ID NO.: 482 |
| HMX3_HUMAN | SEQ ID NO.: 483 |
| HMX1_HUMAN | SEQ ID NO.: 484 |
| KIF22_HUMAN | SEQ ID NO.: 485 |
| CSTF2_HUMAN | SEQ ID NO.: 486 |
| CEBPE_HUMAN | SEQ ID NO.: 487 |
| DLX2_HUMAN | SEQ ID NO.: 488 |
| ZMYM3_HUMAN | SEQ ID NO.: 489 |
| PPARG_HUMAN | SEQ ID NO.: 490 |
| PRIC1_HUMAN | SEQ ID NO.: 491 |
| UNC4_HUMAN | SEQ ID NO.: 492 |
| BARX2_HUMAN | SEQ ID NO.: 493 |
| ALX3_HUMAN | SEQ ID NO.: 494 |
| TCF15_HUMAN | SEQ ID NO.: 495 |
| TERA_HUMAN | SEQ ID NO.: 496 |
| VSX2_HUMAN | SEQ ID NO.: 497 |
| HXD12_HUMAN | SEQ ID NO.: 498 |
| CDX1_HUMAN | SEQ ID NO.: 499 |
| TCF23_HUMAN | SEQ ID NO.: 500 |
| ALX1_HUMAN | SEQ ID NO.: 501 |
| HXA10_HUMAN | SEQ ID NO.: 502 |
| RX_HUMAN | SEQ ID NO.: 503 |
| CXXC5_HUMAN | SEQ ID NO.: 504 |
| SCML1_HUMAN | SEQ ID NO.: 505 |
| NFIL3_HUMAN | SEQ ID NO.: 506 |
| DLX6_HUMAN | SEQ ID NO.: 507 |
| MTG8_HUMAN | SEQ ID NO.: 508 |
| CBX8_HUMAN | SEQ ID NO.: 509 |
| CEBPD_HUMAN | SEQ ID NO.: 510 |
| SEC13_HUMAN | SEQ ID NO.: 511 |
| FIP1_HUMAN | SEQ ID NO.: 512 |
| ALX4_HUMAN | SEQ ID NO.: 513 |
| LHX3_HUMAN | SEQ ID NO.: 514 |
| PRIC2_HUMAN | SEQ ID NO.: 515 |
| MAGI3_HUMAN | SEQ ID NO.: 516 |
| NELL1_HUMAN | SEQ ID NO.: 517 |
| PRRX1_HUMAN | SEQ ID NO.: 518 |
| MTG8R_HUMAN | SEQ ID NO.: 519 |
| RAX2_HUMAN | SEQ ID NO.: 520 |
| DLX3_HUMAN | SEQ ID NO.: 521 |
| DLX1_HUMAN | SEQ ID NO.: 522 |
| NKX26_HUMAN | SEQ ID NO.: 523 |
| NAB1_HUMAN | SEQ ID NO.: 524 |
| SAMD7_HUMAN | SEQ ID NO.: 525 |
| PITX3_HUMAN | SEQ ID NO.: 526 |
| WDR5_HUMAN | SEQ ID NO.: 527 |
| MEOX2_HUMAN | SEQ ID NO.: 528 |
| NAB2_HUMAN | SEQ ID NO.: 529 |
| DHX8_HUMAN | SEQ ID NO.: 530 |
| FOXA2_HUMAN | SEQ ID NO.: 531 |
| CBX6_HUMAN | SEQ ID NO.: 532 |
| EMX2_HUMAN | SEQ ID NO.: 533 |
| CPSF6_HUMAN | SEQ ID NO.: 534 |
| HXC12_HUMAN | SEQ ID NO.: 535 |
| KDM4B_HUMAN | SEQ ID NO.: 536 |
| LMBL3_HUMAN | SEQ ID NO.: 537 |
| PHX2A_HUMAN | SEQ ID NO.: 538 |
| EMX1_HUMAN | SEQ ID NO.: 539 |
| NC2B_HUMAN | SEQ ID NO.: 540 |
| DLX4_HUMAN | SEQ ID NO.: 541 |
| SRY_HUMAN | SEQ ID NO.: 542 |
| ZN777_HUMAN | SEQ ID NO.: 543 |
| NELL1_HUMAN | SEQ ID NO.: 544 |
| ZN398_HUMAN | SEQ ID NO.: 545 |
| GATA3_HUMAN | SEQ ID NO.: 546 |
| BSH_HUMAN | SEQ ID NO.: 547 |
| SF3B4_HUMAN | SEQ ID NO.: 548 |
| TEAD1_HUMAN | SEQ ID NO.: 549 |
| TEAD3_HUMAN | SEQ ID NO.: 550 |
| RGAP1_HUMAN | SEQ ID NO.: 551 |
| PHF1_HUMAN | SEQ ID NO.: 552 |
| FOXA1_HUMAN | SEQ ID NO.: 553 |
| GATA2_HUMAN | SEQ ID NO.: 554 |
| FOXO3_HUMAN | SEQ ID NO.: 555 |
| ZN212_HUMAN | SEQ ID NO.: 556 |
| IRX4_HUMAN | SEQ ID NO.: 557 |
| ZBED6_HUMAN | SEQ ID NO.: 558 |
| LHX4_HUMAN | SEQ ID NO.: 559 |
| SIN3A_HUMAN | SEQ ID NO.: 560 |
| RBBP7_HUMAN | SEQ ID NO.: 561 |
| NKX61_HUMAN | SEQ ID NO.: 562 |
| TRI68_HUMAN | SEQ ID NO.: 563 |
| R51A1_HUMAN | SEQ ID NO.: 564 |
| MB3L1_HUMAN | SEQ ID NO.: 565 |
| DLX5_HUMAN | SEQ ID NO.: 566 |
| NOTC1_HUMAN | SEQ ID NO.: 567 |
| TERF2_HUMAN | SEQ ID NO.: 568 |
| ZN282_HUMAN | SEQ ID NO.: 569 |
| RGS12_HUMAN | SEQ ID NO.: 570 |
| ZN840_HUMAN | SEQ ID NO.: 571 |
| SPI2B_HUMAN | SEQ ID NO.: 572 |
| PAX7_HUMAN | SEQ ID NO.: 573 |
| NKX62_HUMAN | SEQ ID NO.: 574 |
| ASXL2_HUMAN | SEQ ID NO.: 575 |
| FOXO1_HUMAN | SEQ ID NO.: 576 |
| GATA3_HUMAN | SEQ ID NO.: 577 |
| GATA1_HUMAN | SEQ ID NO.: 578 |
| ZMYM5_HUMAN | SEQ ID NO.: 579 |
| ZN783_HUMAN | SEQ ID NO.: 580 |
| SPI2B_HUMAN | SEQ ID NO.: 581 |
| LRP1_HUMAN | SEQ ID NO.: 582 |
| MIXL1_HUMAN | SEQ ID NO.: 583 |
| SGT1_HUMAN | SEQ ID NO.: 584 |
| LMCD1_HUMAN | SEQ ID NO.: 585 |
| CEBPA_HUMAN | SEQ ID NO.: 586 |
| GATA2_HUMAN | SEQ ID NO.: 587 |
| SOX14_HUMAN | SEQ ID NO.: 588 |
| WTIP_HUMAN | SEQ ID NO.: 589 |
| PRP19_HUMAN | SEQ ID NO.: 590 |
| CBX6_HUMAN | SEQ ID NO.: 591 |
| NKX11_HUMAN | SEQ ID NO.: 592 |
| RBBP4_HUMAN | SEQ ID NO.: 593 |
| DMRT2_HUMAN | SEQ ID NO.: 594 |
| SMCA2_HUMAN | SEO ID NO.: 595 |

In some embodiments, an effector domain comprises a functional domain that represses or silences gene expression, and the functional domain is a part of a larger protein, e.g., a zinc finger repressor protein. Functional domains that are capable of modulating gene expression, e.g., repress or increase gene expression can be identified from the larger protein with known methods and methods provided herein. For example, functional effector domains that can reduce or silence target gene expression may be identified based on sequences of repressor or activator proteins. Amino acid sequences of proteins having the function of modulating gene expression may be obtained from available genome browsers, such as UCSD genome browser or Ensembl genome browser. For example, a full length 573 amino acid sequence of the ZNF10 protein is provided in SEQ ID NO.: 596.

Protein annotation databases such as UniProt or Pfam can be used to identify functional domains within the full protein sequence. Using these tools, the repression domain can be identified within the ZNF10 protein sequence. In some instances, various functional domains identified from a larger protein may be tested. Databases may differ in the specific boundary domains. For example, in some embodiments, a repression domain derived from ZNF10 includes amino acids 14-85 of the above referenced ZNF10 sequence. In some embodiments, a repression domain derived from ZNF10 consists of amino acids 14-85 of the above referenced ZNF10 sequence. In some embodiments, a repression domain derived from ZNF10 includes amino acids 13-54 of the above referenced ZNF10 sequence. In some embodiments, a repression domain derived from ZNF10 consists of amino acids 13-54 of the above referenced ZNF10 sequence. As a starting point, the largest sequence, encompassing all regions identified by different databases, may be tested for gene expression modulation activity, for example, a region of the ZN10 protein comprising amino acids 13-85 is tested as a starting point. In further embodiments, the starting point region may be truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids at the N-terminus or C-terminus and various truncations may be tested to identify the minimal functional unit.

In some embodiments, the effector domain comprises a histone deacetylase protein domain. In some embodiments, the effector domain comprises a HDAC family protein domain, for example, a HDAC1, HDAC3, HDAC5, HDAC7, or HDAC9 protein domain. In some embodiments, the effector domain removes the acetyl group. In some embodiments, the effector domain comprises a nucleosome remodeling domain. In some embodiments, the effector domain comprises a nucleosome remodeling and deacetylase complex (NURD), which removes acetyl groups from histones.

In some embodiments, the effector domain comprises a Tripartite motif containing 28 (TRIM28, TIF1-beta, or KAP1) protein. In some embodiments, the effector domain comprises one or more KAP1 protein. The KAP1 protein in an epigenetic editor may form a complex with one or more other effector domains of the epigenetic editor or one or more proteins involved in modulation of gene expression in a cellular environment. For example, KAP1 may be recruited by a KRAB domain of a transcriptional repressor. In some embodiments, KAP1 interacts with or recruits a histone deacetylase protein, a histone-lysine methyltransferase protein (e.g. depositing methyl groups on lysine 9 [K9] of a histone H3 tail [H3K9]), a chromatin remodeling protein, and/or a heterochromatin protein. In some embodiments, a KAP1 protein interacts with or recruits one or more protein complexes that reduces or silences gene expression. In some embodiments, a KAP1 protein interacts with or recruits a heterochromatin protein 1 (HP1) protein (e.g. via a chromoshadow domain of the HP1 protein), a SETDB1 protein, a HDAC protein, and/or a NuRD protein complex component. In some embodiments, a KAP1 protein recruits a CHD3 subunit of the nucleosome remodeling and deacetylation (NuRD) complex, thereby decreasing or silencing expression of a target gene. In some embodiments, a KAP1 protein recruits a SETDB1 protein (e.g. to a promoter region of a target gene), thereby decreasing or silencing expression of the target gene via H3K9 methylation associated with, e.g. the promoter region of the target gene. In some embodiments, recruitment of the SETDB1 protein results in heterochromatinization of a chromosome region harboring the target gene, thereby reducing or silencing expression of the target gene. In some embodiments, a KAP1 protein interacts with or recruits a HP1 protein, thereby decreasing or silencing expression of a target gene via reduced acetylation of H3K9 or H3K14 on histone tails associated with the target gene. Recruitment of SETDB1 induces heterochromatinization. In some embodiments, a KAP1 protein interacts with or recruits a ZFP90 protein (e.g. isoform 2 of ZFP90), and/or a FOXP3 protein.

Amino acid sequence of an exemplary KAP1 protein is provided in SEQ ID NO.: 597.

In some embodiments, the effector domain comprises a protein domain that interacts with or is recruited by one or more DNA epigenetic marks. For example, the effector domain may comprise a methyl CpG binding protein 2 (MECP2) protein that interacts with methylated DNA nucleotides in the target gene. In some embodiments, the MECP2 protein interacts with methylated DNA nucleotides in a CpG island of the target gene. In some embodiments, the MECP2 protein interacts with methylated DNA nucleotides not in a CpG island of the target gene. In some embodiments, the MECP2 protein in an epigenetic editor results in condensed chromatin structure, thereby reducing or silencing expression of the target gene. In some embodiments, the MECP2 protein in an epigenetic editor interacts with a histone deacetylase (e.g. HDAC), thereby repressing or silencing expression of the target gene. In some embodiments, the MECP2 protein in an epigenetic editor blocks access of a transcription factor or transcriptional activator to the target gene, thereby repressing or silencing expression of the target gene.

Amino acid sequence of an exemplary MECP2 protein is provided in SEQ ID NO.: 598.

In some embodiments, an effector domain comprises a chromoshadow domain, a ubiquitin-2 like Rad60 SUMO-like (Rad60-SLD/SUMO) domain, a chromatin organization modifier domain (Chromo) domain, a Yaf2/RYBP C-terminal binding motif domain (YAF2_RYBP), a CBX family C-terminal motif domain (CBX7_C), a Zinc finger C3HC4 type (RING finger) domain (zf-C3HC4_2), a Cytochrome b5 domain (Cyt-b5), a helix-loop-helix domain (HLH), a high mobility group box domain (HMG-box), a Sterile alpha motif domain (SAM_1), basic leucine zipper domain (bZIP_1), a Myb_DNA-binding domain, a Homeodomain, a MYM-type Zinc finger with FCS sequence domain (zf-FCS), a interferon regulatory factor 2-binding protein zinc finger domain (IRF-2BP1_2), a SSX repression domain (SSXRD), a B-box-type zinc finger domain (zf-B_box), a sterile alpha motif domain (SAM_2), a CXXC zinc finger domain (zf-CXXC), a regulator of chromosome condensation 1 domain (RCC1), a SRC homology 3 domain (SH3_9), a sterile alpha motif/Pointed domain (SAM_PNT), a Vestigial/Tondu family domain (Vg_Tdu), a LIM domain, a RNA recognition motif domain (RRM_1), a basic leucine zipper domain (bZIP 2), a paired amphipathic helix domain (PAH), a proteasomal ATPase OB C-terminal domain (Prot_ATP ID_OB), a nervy homology 2 domain (NHR2), a helix-hairpin-helix motif domain (HHH 3), a hinge domain of cleavage stimulation factor subunit 2 (CSTF2 hinge), a PPAR gamma N-terminal region domain (PPARgamma N), a CDC48 N-terminal domain (CDC48_2), a WD40 repeat domain (WD40), a Fip1 motif domain (Fip1), a PDZ domain (PDZ_6), a Von Willebrand factor type C domain (VWC), aNAB conserved region 1 domain (NCD1), a Si RNA-binding domain (Si), a HNF3 C-terminal domain (HNF_C), a Tudor domain (Tudor 2), a histone-like transcription factor (CBF/NF-Y) and archaeal histone domain (CBFD_NFYB HMF), a Zinc finger protein domain (DUF3669), a EGF-like domain (cEGF), a GATA zinc finger domain (GATA), a TEA/ATTS domain (TEA), a phorbol esters/diacylglycerol binding domain (C1-1), polycomb-like MTF2 factor 2 domain (Mtf2_C), a transactivation domain of FOXO protein family (FOXO-TAD), a Homeobox KN domain (Homeobox KN), a BED zinc finger domain (zf-BED), a zinc finger of C3HC4-type RING domain (zf-C3HC4_4), a RAD51 interacting motif domain (RAD51_interact), a p55-binding region of a Methyl-CpG-binding domain protein MBD (MBDa), Notch domain, a Raf-like Ras-binding domain (RBD), a Spin/Ssty family domain (Spin-Ssty), a PHD finger domain (PHD_3), a Low-density lipoprotein receptor domain class A (Ldl_recept_a), a CS domain, a DM DNA binding domain, or a QLQ domain. In some embodiments, the effector domain is a protein domain comprising a YAF2_RYBP domain, or homeodomain or any combination thereof. In some embodiments, the homeodomain of the YAF2_RYBP domain is a PRD domain, a NKL domain, a HOXL domain, or a LIM domain. In some embodiments, the effector domain comprises a protein domain selected from a group consisting of SUMO3 domain, Chromo domain from M phase phosphoprotein 8 (MPP8), chromoshadow domain from Chromobox 1 (CBX1), and SAM_1/SPM domain from Scm Polycomb Group Protein Homolog 1 (SCMH1). In some embodiments, the effector domain comprises a HNF3 C-terminal domain (HNF_C). In some embodiments, the HNF_C domain is from FOXA1 or FOXA2. In some embodiments, the HNF_C domain comprises an EH1 (engrailed homology 1) motif. In some embodiments, the effector domain comprises an interferon regulatory factor 2-binding protein zinc finger domain (IRF-2BP1_2). In some embodiments, the effector domain comprises a Cyt-b5 domain from DNA repair factor HERC2 E3 ligase. In some embodiments, the effector domain comprises a variant SH3 domain (SH3_9) from Bridging Integrator 1 (BIN1). In some embodiments, the effector domain is HMG-box domain from transcription factor TOX or zf-C3HC4-2 RING finger domain from the polycomb component PCGF2. In some embodiment, the effector domain comprises a Chromodomain-helicase-DNA-binding protein 3 (CHD3). In some embodiments, the effector domain comprises a ZNF783 domain. In some embodiments, the effector domain comprises a YAF2_RYBP domain. In some embodiment, the YAF2_RYBP domain comprises a 32 amino acid Yaf2/RYBP C-terminal binding motif domain (32 AA RYBP).

In some embodiments, an effector domain makes an epigenetic modification at a target gene that activates expression of the target gene. In some embodiments, an effector domain modifies the chemical modification of DNA or histone residues associated with the DNA at a target gene harboring the target sequence, thereby activating or increasing expression of the target gene. In some embodiments, the effector domain comprises a DNA demethylase, a DNA dioxygenase, a DNA hydroxylase, or a histone demethylase domain.

In some embodiments, the effector domain comprises a DNA demethylase domain that removes a methyl group from DNA nucleotides, thereby increasing or activating expression of the target gene.

In some embodiments, the effector domain comprises a TET (ten-eleven translocation methylcytosine dioxygenase) family protein domain that demethylates cytosine in methylated form and thereby increases expression of a target gene. In some embodiments, the effector domain comprises a TET1, TET2, or TET3 protein domain or any combination thereof. In some embodiments, the effector domain comprises a TET1 domain. In some embodiments, the effector domain comprises a KDM family protein domain that demethylates lysines in DNA-associated histones, thereby increasing expression of the target gene.

Exemplary demethylase domains that may be part of an epigenetic effector domain are provided in Table 4 below.

TABLE 4
Exemplary demethylase sequences that may
be used in epigenetic effector domains

| Protein | Species | Protein Sequence |
|---|---|---|
| TET1 | Human | SEQ ID NO.: 599 |
| TET2 | Human | SEQ ID NO.: 600 |
| TET3 | Human | SEQ ID NO.: 601 |
| TDG | Human | SEQ ID NO.: 602 |
| ROS1 | Arabidopsis | SEQ ID NO.: 603 |
| DME | Arabidopsis | SEQ ID NO.: 604 |
| DML2 | Arabidopsis | SEQ ID NO.: 605 |
| DML3 | Arabidopsis | SEQ ID NO.: 606 |

The effector domain may activate expression of the target gene. In some embodiments, the effector domain comprises a protein domain that recruits one or more transcription activator domains. In some embodiments, the effector domain comprises a protein domain that recruits one or more transcription factors. In some embodiments, the effector domain comprises a transcription activator or a transcription factor domain. In some embodiments, the effector domain comprises a Herpes Simplex Virus Protein 16 (VP16) activation domain. In some embodiments, the effector domain comprises an activation domain comprising a tandem repeat of multiple VP16 activation domains. In some embodiments, the effector domain comprises four tandem copies of VP16, a VP64 activation domain. In some embodiments, the effector domain comprises a p65 activation domain of NFκB; an Epstein-Barr virus R transactivator (Rta) activation domain. In some embodiments, the effector domain comprises a fusion of multiple activators, e.g., a tripartite activator of the VP64, the p65, and the Rta activation domains, (a VPR activation domain).

In some embodiments, an effector domain comprises a transactivation domain of FOXO protein family (FOXO-TAD), a LMSTEN motif domain (LMSTEN) (“LMSTEN” disclosed as SEQ ID NO: 1163), a Transducer of regulated CREB activity C terminus domain (TORC_C), a QLQ domain, a Nuclear receptor coactivator domain (Nuc_rec_co-act), an Autophagy receptor zinc finger-C2H2 domain (Zn-C2H2-12), an Anaphase-promoting complex subunit 16 (ANAPC16), a Dpy-30 domain, a ANC1 homology domain (AHD), a Signal transducer and activator of transcription 2 C terminal (STAT2_C), a I-kappa-kinase-beta NEMO binding domain (IKKbetaNEMObind), a Early growth response N-terminal domain (DUF3446), a TFIIE beta subunit core domain (TFIIE_beta), a N-terminal domain of DPF2/REQ (Requiem N), a LNR domain (Notch), a Atypical Arm repeat (Arm 3), a Protein kinase C terminal domain (PKinase_C), WW domain, a SH3 domain (SH3_1), a Myb-like DNA-binding domain, a WD domain G-beta repeat (WD40), a PHD-finger (PHD), a RNA recognition motif domain (RRM_1), a GATA zinc finger domain (GATA), a Vps4 C terminal oligomerization domain (Vps4_C), or in any combination thereof. In some embodiments, the effector domain comprises a KRAB domain that activates expression of a target gene. For example the KRAB domain may be a ZNF473 KRAB domain, a ZFP28 KRAB domain, a ZNF496 KRAB domain, or a ZNF597 KRAB domain or any combination thereof. In some embodiments, the KRAB domain comprises a 41-amino-acid ZNF473 KRAB domain (41 AA ZNF473). In some embodiments, the effector domain comprises a FOXO-TAD domain, a LMSTEN domain (“LMSTEN” disclosed as SEQ ID NO: 1163), or a TORC_C domain. In some embodiment, the protein domain comprises a RNA polymerase 64 transcription mediator complex subunit 9 (Med9), TFIIE beta subunit core domain (TFIIED3), nuclear receptor coactivator 3 domain (NCOA3), transactivation domain of FOXO protein family (FOXO-TAD), LMSTEN motifdomain (“LMSTEN” disclosed as SEQ ID NO: 1163), early growth response N-terminal domain (DUF3446), QLQ domain, or Dpy-30 motif domain or any combination thereof. In some embodiment, the effector domain comprises a ZNF473 KRAB domain or a Med9 domain.

Exemplary domains that can activate or increase target gene expression are provided in Table 5 below.

TABLE 5
Exemplary protein domains that may be used in epigenetic
effector domains to increase target gene expression

| Protein | Species | Protein Sequence |
|---|---|---|
| VP16 | Herpes simplex virus type 1 (strain 17) | SEQ ID NO.: 607 |
| VP64 | Herpes simplex virus type 1 | SEQ ID NO.: 608 |
| VP160 | Herpes simplex virus type 1 | SEQ ID NO.: 609 |
| HIF1alpha | Human | SEQ ID NO.: 610 |
| CITED2 | Human | SEQ ID NO.: 611 |
| Stat3 | Human | SEQ ID NO.: 612 |
| p65 | Human | SEQ ID NO.: 613 |
| p53 | Human | SEQ ID NO.: 614 |
| ZNF473 | Human | SEQ ID NO.: 615 |
| FOXO1 | Human | SEQ ID NO.: 616 |
| Myb | Human | SEQ ID NO.: 617 |
| CRTC1 | Human | SEQ ID NO.: 618 |
| Med9 | Human | SEQ ID NO.: 619 |
| EGR3 | Human | SEQ ID NO.: 620 |
| SMARCA2 | Human | SEQ ID NO.: 621 |
| Dpy-30 | Human | SEQ ID NO.: 622 |
| NCOA3 | Human | SEQ ID NO.: 623 |
| ZFP28 | Human | SEQ ID NO.: 624 |
| ZNF496 | Human | SEQ ID NO.: 625 |
| ZNF597 | Human | SEQ ID NO.: 626 |
| HSF1 | Human | SEQ ID NO.: 627 |
| RTA | Epstein-barr virus (strain B95-8) | SEQ ID NO.: 628 |

Additional exemplary domains that can activate or increase target gene expression are provided in Table 6 below.

TABLE 6
Exemplary protein domains that may be used in epigenetic
effector domains to increase target gene expression

| Gene name | Extended Domain sequence |
|---|---|
| ABL1_HUMAN | SEQ ID NO.: 629 |
| AF9_HUMAN | SEQ ID NO.: 630 |
| ANM2_HUMAN | SEQ ID NO.: 631 |
| APBB1_HUMAN | SEQ ID NO.: 632 |
| APC16_HUMAN | SEQ ID NO.: 633 |
| BTK_HUMAN | SEQ ID NO.: 634 |
| CACO1_HUMAN | SEQ ID NO.: 635 |
| CRTC2_HUMAN | SEQ ID NO.: 636 |
| CRTC3_HUMAN | SEQ ID NO.: 637 |
| CXXC1_HUMAN | SEQ ID NO.: 638 |
| DPF1_HUMAN | SEQ ID NO.: 639 |
| DPY30_HUMAN | SEQ ID NO.: 640 |
| EGR3_HUMAN | SEQ ID NO.: 641 |
| ENL_HUMAN | SEQ ID NO.: 642 |
| FIGN_HUMAN | SEQ ID NO.: 643 |
| FOXO1_HUMAN | SEQ ID NO.: 644 |
| FOXO3_HUMAN | SEQ ID NO.: 645 |
| IKKA_HUMAN | SEQ ID NO.: 646 |
| IMA5_HUMAN | SEQ ID NO.: 647 |
| ITCH_HUMAN | SEQ ID NO.: 648 |
| KIBRA_HUMAN | SEQ ID NO.: 649 |
| KPCI_HUMAN | SEQ ID NO.: 650 |
| KS6B2_HUMAN | SEQ ID NO.: 651 |
| MTA3_HUMAN | SEQ ID NO.: 652 |
| MYB_HUMAN | SEQ ID NO.: 653 |
| MYBA_HUMAN | SEQ ID NO.: 654 |
| NCOA2_HUMAN | SEQ ID NO.: 655 |
| NCOA3_HUMAN | SEQ ID NO.: 656 |
| NOTC1_HUMAN | SEQ ID NO.: 657 |
| NOTC1_HUMAN | SEQ ID NO.: 658 |
| NOTC2_HUMAN | SEQ ID NO.: 659 |
| PRP19_HUMAN | SEQ ID NO.: 660 |
| PYGO1_HUMAN | SEQ ID NO.: 661 |
| PYGO2_HUMAN | SEQ ID NO.: 662 |
| SAV1_HUMAN | SEQ ID NO.: 663 |
| SMCA2_HUMAN | SEQ ID NO.: 664 |
| SMRC2_HUMAN | SEQ ID NO.: 665 |
| STAT2_HUMAN | SEQ ID NO.: 666 |
| T2EB_HUMAN | SEQ ID NO.: 667 |
| U2AF4_HUMAN | SEQ ID NO.: 668 |
| WBP4_HUMAN | SEQ ID NO.: 669 |
| WWP1_HUMAN | SEQ ID NO.: 670 |
| WWP2_HUMAN | SEQ ID NO.: 671 |
| WWTR1_HUMAN | SEQ ID NO.: 672 |
| ZFP28_HUMAN | SEQ ID NO.: 673 |
| ZN473_HUMAN | SEQ ID NO.: 674 |
| ZN496_HUMAN | SEQ ID NO.: 675 |
| ZN597_HUMAN | SEQ ID NO.: 676 |

In some embodiments, an effector domain regulates acetylation of a histone associated with the target gene. In some embodiments, the effector domain comprises a histone acetyltransferase domain. In some embodiments, the effector domain comprises a protein domain that interacts with a histone acetyltransferase domain to effect histone acetylation. In some embodiments, the effector domain comprises a histone acetyltransferase 1 (HAT1) domain. In some embodiments, the effector domain comprises a histone acetyltransferase (HAT) core domain of the human E1A-associated protein p300. In some embodiments, the effector domain comprises a CBP/p300 histone acetyltransferase or a catalytic domain thereof. In some embodiments, the effector domain comprises a CREBBP, GCN4, GCN5, SAGA, SALSA, HAP2, HAP3, HAP4, PCAF, KMT2A, or any combination thereof.

Sequences of exemplary histone acetyltransferase domains are provided below: Exemplary p300 amino acid sequence: SEQ ID NO.: 677.

Exemplary CREBBP amino acid sequence: SEQ ID NO.: 678.

In some embodiments, an epigenetic editor described herein alters chemical modification of a target gene that harbors the target sequence. For example, an epigenetic editor comprising a methyltransferase domain can methylate the DNA or histone residues of the target gene, at nucleotides (or histones) near the target sequence, or within 100, 200, 300, 400, 500, 600, 700, 800, 900, 1000, 1500, 2000, 2500, 3000 base pairs flanking the target sequence, thereby repress or silent expression of the target gene. An epigenetic editor comprising a DNA or histone demethylase can remove the methylation of the DNA or histone residues associated with or bound to the target gene, thereby activating or increasing expression of the target gene.

Chemical modifications mediated by an epigenetic editor may be near a target sequence of a target gene. For example, such modifications may occur within 50, 100, 200, 300, 400, 500, 600, 700, 800, 900, 1000 base pairs flanking the target sequence. In some embodiments, the chemical modification occurs within 50, 100, 200, 300, 400, 500, 600, 700, 800, 900, 1000 base pairs upstream of the 5′ end of the target sequence.

### Epigenetic Editors

Described herein are epigenetic editors for epigenetic modification and expression regulation of target genes. As used herein, an epigenetic editor can be any agent that binds a target polynucleotide and has epigenetic modulation activity. In some embodiments, the epigenetic editor binds the polynucleotide at a specific sequence using a DNA binding domain. In some embodiments, the epigenetic editor binds the polynucleotide at a specific sequence using a nucleic acid guided DNA binding protein. In some embodiments, the epigenetic editor comprises an effector domain capable of modulating epigenetic state of a nucleic acid sequence at or adjacent to the target polynucleotide. In some embodiments, the epigenetic editor is capable of depositing an epigenetic editing mark on a chromatin region, a nucleic acid sequence, or a histone amino acid residue, at or adjacent to the target polynucleotide. For example, the epigenetic editor can be capable of methylating, demethylating, acetylating, deacetylating, ubiquitinating or deubiquitinating a chromatin region, a nucleic acid sequence, or a histone amino acid residue, at or adjacent to the target polynucleotide. In some embodiments, the epigenetic editor is capable of recruiting one or more proteins or complexes involved in transcription regulation, for example, a transcription factor, a transcription activator, a transcription repressor, or an insulator to a chromatin region, a nucleic acid sequence, or a histone amino acid residue, at or adjacent to the target polynucleotide.

Epigenetic editors provided herein can comprise one or more effector domains as described. In some embodiments, an epigenetic editor comprises multiple effector domains. In some embodiments, an epigenetic editor comprises one effector domain. In some embodiments, the epigenetic editor comprises at least 2, 3, 4, 5, 6, 7, 8, 9, 10 or more effector domains. In some embodiments, the epigenetic editor comprises at least 2 effector domains, e.g., two repressor domains. In some embodiments, the epigenetic editor comprises at least 2 effector domains. In some embodiments, the epigenetic editor comprises two or more effector domains. In some embodiments, the two or more effector domains function synergistically to result in enhanced modulation of a target gene. For example, an epigenetic editor may comprise two effector domains, one of which induces histone deacetylation and the other results in DNA methylation of the target gene.

In some embodiments, an epigenetic editor comprises a DNA methylation domain and a histone deacetylation domain. In some embodiments, an epigenetic editor comprises a DNA methylation domain and a repression domain that recruits additional DNA methylation, histone methylation, or histone deacetylation proteins. In some embodiments, an epigenetic editor comprises a DNA methylation domain and a scaffold protein that recruits additional DNA methylation, histone methylation, or histone deacetylation proteins. In some embodiments, an epigenetic editor comprises a DNA methylation domain, a histone deacetylation domain, and a scaffold protein that recruits additional DNA methylation, histone methylation, or histone deacetylation proteins. In some embodiments, an epigenetic editor comprises two or more DNA methylation domains, a histone deacetylation domain, and a scaffold protein that recruits additional DNA methylation, histone methylation, or histone deacetylation proteins. In some embodiments, an epigenetic editor comprises two or more DNA methylation domains, two or more histone deacetylation domains, and/or two or more scaffold proteins that recruits additional DNA methylation, histone methylation, or histone deacetylation proteins. In some embodiments, the epigenetic editor comprises a KRAB domain and a DNMT3 domain, both of which may synergistically effect enhanced reduction or silencing of expression of a target gene, as compared to an epigenetic effector having only one of the two repressor domains. In some embodiments, the epigenetic editor comprises a KRAB domain, a Dnmt3A domain, and a Dnmt3L domain. In some embodiments, the epigenetic editor comprises the configuration of a DNA binding domain flanked by a KRAB domain and a Dnmt3A-Dnmt3L fusion protein domain. In some embodiments, the epigenetic editor comprises the following configuration: N-[KRAB]-[DNA binding domain]-[Dnmt3A-Dnmt3L]-C, where “]-[” is any nuclear localization signal, any tag sequence, or any linker as provided herein.

In some embodiments, an epigenetic editor comprises a DNA demethylation domain and a histone acetylation domain. In some embodiments, an epigenetic editor comprises a DNA demethylation domain and an activation domain that recruits additional DNA demethylation or histone acetylation proteins. In some embodiments, an epigenetic editor comprises a DNA demethylation domain, a histone acetylation domain, and a scaffold protein that recruits additional DNA demethylation or histone acetylation proteins. In some embodiments, an epigenetic editor comprises two or more DNA demethylation domains, two or more histone acetylation domains, and/or two or more scaffold proteins that recruits additional DNA demethylation or histone deacetylation proteins.

In some embodiments, an epigenetic editor may comprise a VP64 activation domain, a p65 activation domain, and a Rta activation domains (together, a VPR activation domain), all of which synergistically effect enhanced activation of expression of a target gene, as compared to an epigenetic effector having only one of the three activation domains.

An effector domain of an epigenetic editor can be linked to another effector domain via direct fusion, or via any linker as described herein. An effector domain and a DNA binding domain of the epigenetic editor can also be linked via direct fusion or any linker as described herein.

In some embodiments, the two or more effector domains are identical. In some embodiments, the two or more effector domains belong to the same protein family. In some embodiments, the two or more effector domains are different proteins involved in the same transcriptional machinery or regulatory mechanism.

Multiple epigenetic editors, e.g. epigenetic editor fusion proteins or complexes may be used to effect activation or repression of a target gene or multiple target genes. For example, an epigenetic editor fusion protein comprising a DNA binding domain (e.g. dCas9 domain) and a methylation domain may be co-delivered with two or more guide RNAs, each targeting a different target DNA sequence. The two or more target DNA sequences may be in the same target gene, or may be in different target genes. The two or more target DNA sequences recognized by the DNA-binding domain may be overlapping or non-overlapping. The target sites for two of the DNA-binding domains may be separated by, for example, about 100 base pairs, about 200 base pairs, about 300 base pairs, about 400 base pairs, about 500 base pairs, about 600 or more base pairs. In addition, when targeting double-stranded DNA, such as an endogenous genome, the DNA-binding domains of the artificial transcription factors may target the same or different strands (one or more to positive strand and/or one or more to negative strand). Further, the same or different DNA-binding domains may be used in the epigenetic editors described herein.

### Linkers

Epigenetic editors provided herein may comprise one or more linkers that connect one or more components of the epigenetic editors. A linker may be a covalent bond or a polymeric linker with many atoms in length. A linker may be a peptide linker or a non-peptide linker.

In certain embodiments, linkers may be used to link any of the peptides or peptide domains of the epigenetic editor. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker is a non-peptide linker. For example, the linker may be a carbon bond, a disulfide bond, or carbon-heteroatom bond. In certain embodiments, the linker is a carbon-nitrogen bond of an amide linkage. In certain embodiments, the linker is a cyclic or acyclic, substituted or unsubstituted, branched or unbranched aliphatic or heteroaliphatic linker.

In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, alkyl halides, aryl halides, acyl halides, and isothiocyanates.

In some embodiments, one or more linkers of an epigenetic editor provided herein is a peptide linker. For example, a zinc finger array and a repressor domain may be connected by a peptide linker, forming a zinc finger-repressor fusion protein. A peptide linker can be any length applicable to the epigenetic editor fusion proteins described herein. In some embodiments, the linker can comprise a peptide between 1 and 200 amino acids. In some embodiments, a DNA binding domain, e.g., a zinc finger array and an effector domain are fused via a linker that comprises from 1 to 5, 1 to 10, 1 to 20, 1 to 30, 1 to 40, 1 to 50, 1 to 60, 1 to 80, 1 to 100, 1 to 150, 1 to 200, 5 to 10, 5 to 20, 5 to 30, 5 to 40, 5 to 60, 5 to 80, 5 to 100, 5 to 150, 5 to 200, 10 to 20, 10 to 30, 10 to 40, 10 to 50, 10 to 60, 10 to 80, 10 to 100, 10 to 150, 10 to 200, 20 to 30, 20 to 40, 20 to 50, 20 to 60, 20 to 80, 20 to 100, 20 to 150, 20 to 200, 30 to 40, 30 to 50, 30 to 60, 30 to 80, 30 to 100, 30 to 150, 30 to 200, 40 to 50, 40 to 60, 40 to 80, 40 to 100, 40 to 150, 40 to 200, 50 to 60 50 to 80, 50 to 100, 50 to 150, 50 to 200, 60 to 80, 60 to 100, 60 to 150, 60 to 200, 80 to 100, 80 to 150, 80 to 200, 100 to 150, 100 to 200, or 150 to 200 amino acids in length. Longer or shorter linkers are also contemplated. In some embodiments, the peptide linker is 4, 16, 32, or 104 amino acids in length. In some embodiments, the peptide linker is a flexible linker. In some embodiments, the peptide linker is a rigid linker.

In some embodiments, the peptide linker comprises the amino acid sequence of SEQ ID NO.: 679-683

In some embodiments, the peptide linker is a XTEN linker. In some embodiments, the peptide linker comprises the amino acid sequence SEQ ID NO.: 684. In some embodiments, the linker is 24 amino acids in length. In some embodiments, the linker comprises the amino acid sequence SEQ ID NO.: 685. In some embodiments, the linker is 40 amino acids in length. In some embodiments, the linker comprises the amino acid sequence SEQ ID NO.: 686. In some embodiments, the linker is 64 amino acids in length. In some embodiments, the linker comprises the amino acid sequence SEQ ID NO.: 687.

In some embodiments, the linker is 92 amino acids in length. In some embodiments, the linker comprises the amino acid sequence SEQ ID NO.: 688.

Various linker lengths and flexibilities between a effector domain (e.g., a repressor domain) and a DNA binding protein (e.g., a Cas9 domain), between a effector domain and a second effector domain, or between any two components of an epigenetic editor can be employed (e.g., ranging from very flexible linkers of the form (GGGGS)n (SEQ ID NO: 1159), (GGGGS)n (SEQ ID NO: 1159), and (G)n to more rigid linkers of the form (EAAAK)n (SEQ ID NO: 1160), (SGGS)n (SEQ ID NO: 1161), and (XP)n) in order to achieve the optimal length for effector domain activity for the specific application. In some embodiments, n is any integer between 3 and 30. In some embodiments, n is 1, 2, 3, 4, 5, 6, 7, 8, 9, 10, 11, 12, 13, 14, or 15. In some embodiments, the linker comprises a (GGS)n motif, wherein n is 1, 3, or 7 (SEQ ID NO: 1164).

In some embodiments, a linker in an epigenetic editor comprises a nuclear localization signal, for example, of peptide sequence SEQ ID NO.: 689-694. In some embodiments, a linker in an epigenetic editor comprises a cleavable peptide, e.g., a T2A peptide, a p2A peptide, or a furin/p2A peptide. In some embodiments, a linker in an epigenetic editor comprises an expression tag, e.g. a detectable tag such as a green fluorescence protein.

In some embodiments, a linker comprises a nucleic acid. For example, one or more linkers of an epigenetic editor may include a nucleic acid that is capable of binding to, interacting with, associating with, or forming a complex with a polypeptide. In some embodiments, the nucleic acid linker may be a RNA linker capable of binding to and/or interacting with a RNA binding protein domain, e.g. a phase derived RNA binding domain. In some embodiments, the nucleic acid linker may be fused to a guide polynucleotide capable of binding to a Cas protein of an epigenetic editor. In some embodiments, the nucleic acid linker comprises a K homology (KH) domain binding sequence, a MS2 coat protein binding sequence, a PP7 coat protein binding sequence, a SfMu COM coat protein binding sequence, a telomerase Ku binding motif binding sequence, a sm7 protein binding sequence, or other RNA recognition motif binding sequence thereof.

In some embodiments, a linker comprises an affinity domain that specifically binds a component of an epigenetic effector. For example, an epigenetic effector may comprise a programmable DNA binding domain, a linker comprising an affinity domain having specific binding affinity to an epigenetic effector domain. The affinity domain may comprise an antibody, a single chain antibody, a nanobody, and antigen binding sequence, an antibody, a nanobody, a functional antibody fragment, a single chain variable fragment (scFv), an Fab, a single-domain antibody (sdAb), a VH domain, a VL domain, a VNAR domain, a VHH domain, a bispecific antibody, a diabody, or a functional fragment or a combination thereof. In some embodiments, an epigenetic effector domain comprises a programmable DNA binding domain and a KAP1 antibody which binds to a KAP1 protein. In some embodiments, an epigenetic effector domain comprises a programmable DNA binding domain and a KRAB antibody which binds to a KRAB protein. In some embodiments, an epigenetic effector domain comprises a programmable DNA binding domain and a DNMT1 antibody which binds to a DNMT1 protein. In some embodiments, an epigenetic effector domain comprises a programmable DNA binding domain and a DNMT3A antibody which binds to a DNMT3A protein. In some embodiments, an epigenetic effector domain comprises a programmable DNA binding domain and a DNMT3L antibody which binds to a DNMT3L protein. In some embodiments, an epigenetic effector domain comprises a programmable DNA binding domain and a ZIM3 antibody which binds to a ZIM3 protein. In some embodiments, an epigenetic effector domain comprises a programmable DNA binding domain and a TET1 antibody which binds to a TET1 protein. In some embodiments, an epigenetic effector domain comprises a programmable DNA binding domain and a VP16 or VP64 antibody which binds to a VP16 or VP64 protein.

In some embodiments, a linker comprises a repeat peptide array. In some embodiments, a linker comprises an epitope tag, for example, a SunTag. In some embodiments, an epigenetic editor comprises one or more peptide arrays comprising multiple copies of an epitope tag that can link multiple effector domains attached to or fused to peptide recognizing the epitope tag. For example, a epitope tag array can link a DNA binding domain and multiple effector domains or multiple copies of effector domains fused to or attached to antibody sequences recognizing the epitope tag. In some embodiments, an epigenetic editor comprises at least 1, 2, 3, 4, 5, 6, 7, 8, 9, 10 or more epitope tag repeats that link at least 1, 2, 3, 4, 5, 6, 7, 8, 9, 10 or more effector domains or copies of effector domains. In some embodiments, an epigenetic editor comprises multiple epitope tag repeats that link multiple effector domains and detectable expression tag domains, e.g. GFPs. In some embodiments, the repeat peptide array comprises gene control non-depressible 4 (GCN4) peptide sequences. In some embodiments, the repeat peptide arrays are further linked by linking peptide sequences of 15 to 50 amino acids. Repeat peptide arrays as described in US patent application No. US20170219596 and U.S. Pat. No. 10,612,044 are incorporated herein by reference in its entirety.

### Nuclear Localization Signals

In some embodiments, the epigenetic editors provided herein comprise one or more nuclear targeting sequences. For example, a zinc finger—repressor fusion protein described herein may further comprise one or more nuclear targeting sequences, for example, a nuclear localization sequence (NLS). In some embodiments, the fusion protein comprises multiple NLSs. In some embodiments, the fusion protein comprises a NLS at the N-terminus or the C-terminus of the fusion protein. In some embodiments, the fusion protein comprises a NLS at both the N-terminus and the C-terminus. In some embodiments, the NLS is embedded in the middle of the fusion protein. In some embodiments, a NLS comprises an amino acid sequence that facilitates the importation of a protein, that comprises an NLS, into the cell nucleus. In some embodiments, the NLS is fused to the N-terminus of the fusion protein. In some embodiments, the NLS is fused to the C-terminus of the fusion protein. In some embodiments, the NLS is fused to the N-terminus of the nucleic acid binding protein, e.g. the Cas9 or zinc finger array. In some embodiments, the NLS is fused to the C-terminus of the nucleic acid binding protein. In some embodiments, the NLS is fused to the N-terminus of a effector domain, e.g., a repressor domain. In some embodiments, the NLS is fused to the C-terminus of a effector domain, e.g., a repressor domain. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. In some embodiments, a NLS comprises the amino acid sequence SEQ ID NO.: 687 or SEQ ID NO.: 692. Additional nuclear localization sequences are known in the art and would be apparent to the skilled artisan.

### Tags

Epigenetic editors provided herein may comprise one or more additional sequences domains, tags, for tracking, detection, and localization of the editors. In some embodiments, an epigenetic editor comprises one or more detectable tags. In some embodiments, the epigenetic editor comprises 1, 2, 3, 4, 5, 6, 7, 8, 9, 10 or more detectable tags. Each of the detectable tags may be same or different.

For example, an epigenetic editor fusion protein may comprise cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art.

In some embodiments, an epigenetic editor comprises from 1 to 2 detectable tags. In aspects, the fusion protein comprises 1 detectable tag. In aspects, the fusion protein comprises 2 detectable tags. In aspects, the fusion protein comprises 3 detectable tags. In aspects, the fusion protein comprises 4 detectable tags. In aspects, the fusion protein comprises 5 detectable tags.

### Epigenetic Editor Structure

The multiple components of epigenetic editors described herein may be in any order. In some embodiments, an epigenetic editor comprises the structure: N′]-[D1]-[D2]-[C′, wherein any one of D1 and D2 is a DNA binding domain or an effector domain.

In some embodiments, an epigenetic editor comprises the structure: N′]-[D1]-[D2]-[D3]-[C′, wherein any one of D1, D2, and D3 is a DNA binding domain, or an effector domain. In some embodiments, D1 is a DNA binding domain. In some embodiments, D2 is a DNA binding domain. In some embodiments, D3 is a DNA binding domain. In some embodiments, D1 is the only DNA binding domain. In some embodiments, D2 is the only DNA binding domain. In some embodiments, D3 is the only DNA binding domain.

In some embodiments, an epigenetic editor comprises the structure: N′]-[D1]-[D2]-[D3]-[D4]-[C′, wherein any one of D1, D2, D3, and D4 is a DNA binding domain, or an effector domain. In some embodiments, D1 is a DNA binding domain. In some embodiments, D2 is a DNA binding domain. In some embodiments, D3 is a DNA binding domain. In some embodiments, D4 is a DNA binding domain. In some embodiments, D1 is the only DNA binding domain. In some embodiments, D2 is the only DNA binding domain. In some embodiments, D3 is the only DNA binding domain. In some embodiments, D4 is the only DNA binding domain.

In some embodiments, an epigenetic editor comprises the structure: N′]-[D1]-[D2]-[D3]-[D4]-[D5]-[C′, wherein any one of D1, D2, D3, D4, and D5 is a DNA binding domain, or an effector domain. In some embodiments, D1 is a DNA binding domain. In some embodiments, D2 is a DNA binding domain. In some embodiments, D3 is a DNA binding domain. In some embodiments, D4 is a DNA binding domain. In some embodiments, D5 is a DNA binding domain. In some embodiments, D1 is the only DNA binding domain. In some embodiments, D2 is the only DNA binding domain. In some embodiments, D3 is the only DNA binding domain. In some embodiments, D4 is the only DNA binding domain. In some embodiments, D5 is the only DNA binding domain.

In some embodiments, the epigenetic editor comprises at least one effector domain that is a DNMT domain. In some embodiments, the epigenetic editor comprises at least one effector domain that is a KRAB domain. In some embodiments, the epigenetic effector comprises at least one effector domain that is a fusion of a DNMT3A-DNMT3L domain.

In some embodiments, the epigenetic editor comprises at least one effector domain that is a TET1 domain. In some embodiments, the epigenetic editor comprises at least one effector domain that is a VP16 domain. In some embodiments, the epigenetic editor comprises at least one effector domain that is a VP64 domain. In some embodiments, the epigenetic editor comprises at least one effector domain that is a RTA domain.

Components of an epigenetic editor may be structured in different configurations. For example, the DNA binding domain may be at the C terminus, the N terminus, or in between two or more epigenetic effector domains or additional domains. In some embodiments, the DNA binding domain is at the C terminus of the epigenetic editor. In some embodiments, the DNA binding domain is at the N terminus of the epigenetic editor. In some embodiments, the DNA binding domain is linked to one or more nuclear localization signals. In some embodiments, the DNA binding domain is linked to two or more nuclear localization signals. In some embodiments, the DNA binding domain is flanked by an epigenetic effector domain or an additional domain on both termini. In some embodiments, the epigenetic editor comprises the configuration of N′]-[epigenetic effector domain 1]-[DNA binding domain]-[epigenetic effector domain 2]-[C′. In some embodiments, the epigenetic editor comprises the configuration of N′]-[epigenetic effector domain 1]-[DNA binding domain]-[epigenetic effector domain 2]-[epigenetic effector domain 3]-[C′. In some embodiments, the epigenetic editor comprises the configuration of N′]-[epigenetic effector domain 1]-[epigenetic effector domain 2]-[DNA binding domain]-[epigenetic effector domain 3]-[C′. In some embodiments, the epigenetic editor comprises the configuration of N′]-[epigenetic effector domain 1]-[epigenetic effector domain 2]-[DNA binding domain]-[epigenetic effector domain 3]-[epigenetic effector domain 4]-[C′. In some embodiments, the epigenetic editor comprises the configuration of N′]-[KRAB]-[DNA binding domain]-[Dnmt3A]-[C′. In some embodiments, the epigenetic editor comprises the configuration of N′]-[KRAB]-[DNA binding domain]-[Dnmt3A]-[Dnmt3L]-[C′. In some embodiments, the epigenetic editor comprises the configuration of N′]-[SETDB1]-[DNA binding domain]-[Dnmt3A]-[Dnmt3L]-[C′. In some embodiments, the epigenetic editor comprises the configuration of N′]-[SETDB1]-[DNA binding domain]-[Dnmt3A]-[C′. In some embodiments, the epigenetic editor comprises the configuration of N′]-[KRAB]-[DNA binding domain]-[Dnmt3A-Dnmt3L]-[C′, wherein Dnmt3A and Dnmt3L are directly fused via a peptide bond.

In some embodiments, the epigenetic editor comprises the configuration of N′]-[Dnmt3A]-[DNA binding domain]-[KRAB]-[C′. In some embodiments, the epigenetic editor comprises the configuration of N′]-[Dnmt3A]-[Dnmt3L]-[DNA binding domain]-[KRAB]-[C′. In some embodiments, the epigenetic editor comprises the configuration of N′]-[Dnmt3A-Dnmt3L]-[DNA binding domain]-[KRAB]-[C′, wherein Dnmt3A and Dnmt3L are directly fused via a peptide bond. In some embodiments, the epigenetic editor comprises the configuration of N′]-[Dnmt3A]-[DNA binding domain]-[SETDB1]-[C′. In some embodiments, the epigenetic editor comprises the configuration of N′]-[Dnmt3A]-[Dnmt3L]-[DNA binding domain]-[SETDB1]-[C′. In some embodiments, the epigenetic editor comprises the configuration of N′]-[Dnmt3A-Dnmt3L]-[DNA binding domain]-[SETDB1]-[C′, wherein Dnmt3A and Dnmt3L are directly fused via a peptide bond. In some embodiments, a connecting structure “]-[” in any one of the epigenetic editor structures is a linker, e.g., a peptide linker. In some embodiments, a connecting structure “]-[” in any one of the epigenetic editor structures is a detectable tag. In some embodiments, a connecting structure “]-[” in any one of the epigenetic editor structures is a peptide bond. In some embodiments, a connecting structure “]-[” in any one of the epigenetic editor structures is a nuclear localization signal. In some embodiments, a connecting structure “]-[” in any one of the epigenetic editor structures is a promoter or a regulatory sequence. In an epigenetic editor structure, the multiple connecting structures “]-[” may be same or may each be a different linker, tag, NLS, or peptide bond.

The DNA binding domain (DBD) of an epigenetic editor may comprise any one of the DNA binding domains described herein or known to those skilled in the art. In some embodiments, the DBD comprises one or more zinc finger arrays. In some embodiments, the DBD comprises a TALE DNA binding domain. In some embodiments, the DBD is a RNA guided programmable DNA binding domain, e.g. a CRISPR-Cas protein domain. Suitable Cas proteins has been provided herein, including nuclease inactive Cas proteins for the purpose of epigenetic editing without causing target DNA strand breaks. A Cas protein in an epigenetic editor may be a nuclease inactive Cas9 (dCas9), a SaCas9d, a SpCas9d, a dCas9 with modified PAM specificity, a high-fidelity dCas9, a nuclease inactive Cpf1 (dCpf1), a dCpf1 with modified PAM specificity, a high-fidelity dCpf1, a dCas12e, a dCasY, or any other Cas protein as described herein.

In some embodiments, an epigenetic editor comprises a DNA binding domain (DBD) and an effector domain that represses or silences expression of a target gene. In some embodiments, the epigenetic editor comprises the configuration of N′]-[repression domain]-[DBD]-[-C′, wherein the connecting structure]-[is any one of the linkers as described herein, a detectable tag, an affinity domain, a peptide bond, a nuclear localization signal, a promoter, and/or a regulatory sequence. In some embodiments, the epigenetic editor comprises the configuration of N′]-[DBD]-[repression domain]-[-C′, wherein the connecting structure]-[is any one of the linkers as described herein, a detectable tag, an affinity domain, a peptide bond, a nuclear localization signal, a promoter, and/or a regulatory sequence.

In some embodiments, an epigenetic editor comprises a DNA binding domain (DBD) and a DNA methyltransferase domain that deposits one or more methylation marks at a target gene, thereby repressing or silencing expression of the target gene. In some embodiments, the epigenetic editor comprises the configuration of N′]-[DNA methyltransferase domain]-[DBD]-[-C′, wherein the connecting structure]-[is any one of the linkers as described herein, a detectable tag, an affinity domain, a peptide bond, a nuclear localization signal, a promoter, and/or a regulatory sequence. In some embodiments, the epigenetic editor comprises the configuration of N′]-[DBD]-[DNA methyltransferase domain]-[-C′, wherein the connecting structure ]-[is any one of the linkers as described herein, a detectable tag, an affinity domain, a peptide bond, a nuclear localization signal, a promoter, and/or a regulatory sequence.

In some embodiments, an epigenetic editor comprises a DNA binding domain (DBD), a DNA methyltransferase domain, and an effector domain that represses or silences expression of a target gene. In some embodiments, the epigenetic editor comprises the configuration of N′]-[DNA methyltransferase domain]-[DBD]-[repression domain]-[C′, wherein the connecting structure]-[is any one of the linkers as described herein, a detectable tag, an affinity domain, a peptide bond, a nuclear localization signal, a promoter, and/or a regulatory sequence. In some embodiments, the epigenetic editor comprises the configuration of N′]-[repression domain]-[DBD]-[DNA methyltransferase domain]-[C′, wherein the connecting structure]-[is any one of the linkers as described herein, a detectable tag, an affinity domain, a peptide bond, a nuclear localization signal, a promoter, and/or a regulatory sequence.

In some embodiments, the epigenetic editor comprises the configuration of N′]-[DNA methyltransferase domain]-[repression domain]-[DBD]-[C′, wherein the connecting structure]-[is any one of the linkers as described herein, a detectable tag, an affinity domain, a peptide bond, a nuclear localization signal, a promoter, and/or a regulatory sequence. In some embodiments, the epigenetic editor comprises the configuration of N′]-[repression domain]-[DNA methyltransferase domain]-[DBD]-[C′, wherein the connecting structure]-[is any one of the linkers as described herein, a detectable tag, an affinity domain, a peptide bond, a nuclear localization signal, a promoter, and/or a regulatory sequence.

The repression domain in an epigenetic editor may comprise any one of the expression repression proteins known to those skilled in the art and as described herein, or any homologs or combination thereof. In some embodiments, the repression domain comprises a histone deacetylase domain. In some embodiments, the repression domain interacts with a scaffold protein domain that recruits one or more protein domains that repress expression of the target gene. For example, the repression domain may recruit or interact with a scaffold protein domain that recruits a PRMT protein, a HDAC protein, a SETDB1 protein, or a NuRD protein domain. In some embodiments, the repression domain interacts with epigenetically marked DNA nucleotides in a target gene thereby repressing or silencing expression of the target gene. In some embodiments, the repression domain comprises a MECP2 domain. In some embodiments, the repression domain comprises a KAP1 domain. In some embodiments, the repression domain comprises any one of the domains of Table 2 or Table 3, or any combination or homologs thereof.

The DNA methyltransferase domain in an epigenetic editor may comprise any one of the DNA methyltransferase proteins known to those skilled in the art and as described herein, or any homologs or combination thereof. In some embodiments, the effector domain comprises a DNMT3 domain. In some embodiments, the DNA methyltransferase domain comprises a DNMT3A domain. In some embodiments, the DNA methyltransferase domain comprises a DNMT3B domain. In some embodiments, the DNA methyltransferase domain comprises a DNMT3C domain. In some embodiments, the DNA methyltransferase domain comprises a DNMT3L domain. In some embodiments, the DNA methyltransferase domain comprises a fusion of DNMT3A-DNMT3L domain. As described herein, the DNMT3A-DNMT3L fusion domain may be in either order, e.g., N-DNMT3A-DNMT3L-C, or N-DNMT3L-DNMT3A-C. In some embodiments, the DNA methyltransferase domain comprises any one of the domains of Table 1, or any combination or homologs thereof.

In some embodiments, an epigenetic editor comprises a DNA binding domain (DBD) and an effector domain that increases expression of a target gene. In some embodiments, the epigenetic editor comprises the configuration of N′]-[activation domain]-[DBD]-[C′, wherein the connecting structure]-[is any one of the linkers as described herein, a detectable tag, an affinity domain, a peptide bond, a nuclear localization signal, a promoter, and/or a regulatory sequence. In some embodiments, the epigenetic editor comprises the configuration of N′]-[DBD]-[activation domain]-[C′, wherein the connecting structure]-[is any one of the linkers as described herein, a detectable tag, an affinity domain, a peptide bond, a nuclear localization signal, a promoter, and/or a regulatory sequence.

In some embodiments, an epigenetic editor comprises a DNA binding domain (DBD) and a DNA demethylation domain that removes one or more methylation marks at a target gene, thereby increasing expression of the target gene. In some embodiments, the epigenetic editor comprises the configuration of N′]-[DNA demethylase domain]-[DBD]-[C′, wherein the connecting structure]-[is any one of the linkers as described herein, a detectable tag, an affinity domain, a peptide bond, a nuclear localization signal, a promoter, and/or a regulatory sequence. In some embodiments, the epigenetic editor comprises the configuration of N′]-[DBD]-[DNA demethylase domain]-[C′, wherein the connecting structure]-[is any one of the linkers as described herein, a detectable tag, an affinity domain, a peptide bond, a nuclear localization signal, a promoter, and/or a regulatory sequence.

In some embodiments, an epigenetic editor comprises a DNA binding domain (DBD), a DNA demethylase domain, and an activation effector domain that increases expression of a target gene. In some embodiments, the epigenetic editor comprises the configuration of N′]-[DNA demethylase domain]-[DBD]-[activation domain]-[C′, wherein the connecting structure]-[is any one of the linkers as described herein, a detectable tag, an affinity domain, a peptide bond, a nuclear localization signal, a promoter, and/or a regulatory sequence. In some embodiments, the epigenetic editor comprises the configuration of N′]-[activation domain]-[DBD]-[DNA demethylase domain]-[C′, wherein the connecting structure]-[is any one of the linkers as described herein, a detectable tag, an affinity domain, a peptide bond, a nuclear localization signal, a promoter, and/or a regulatory sequence.

In some embodiments, the epigenetic editor comprises the configuration of N′]-[DNA demethylase domain]-[activation domain]-[DBD]-[C′, wherein the connecting structure]-[is any one of the linkers as described herein, a detectable tag, an affinity domain, a peptide bond, a nuclear localization signal, a promoter, and/or a regulatory sequence. In some embodiments, the epigenetic editor comprises the configuration of N′]-[activation domain]-[DNA demethylase domain]-[DBD]-[C′, wherein the connecting structure]-[is any one of the linkers as described herein, a detectable tag, an affinity domain, a peptide bond, a nuclear localization signal, a promoter, and/or a regulatory sequence.

The activation domain in an epigenetic editor may comprise any one of the expression activation proteins known to those skilled in the art and as described herein, or any homologs or combination thereof. In some embodiments, the activation domain comprises a histone acetyltransferase domain. In some embodiments, the activation domain interacts with a scaffold protein domain that recruits one or more protein domains that activate expression of the target gene. For example, the activation domain may recruit or interact with a scaffold protein domain that recruits one or more transcription factors or activators. In some embodiments, the activation domain comprises a Herpes Simplex Virus Protein 16 (VP16) activation domain. In some embodiments, the activation domain comprises an activation domain comprising a tandem repeat of multiple VP16 activation domains. In some embodiments, the activation domain comprises four tandem copies of VP16, a VP64 activation domain. In some embodiments, the activation domain comprises eight tandem copies of VP16, a VP128 activation domain. In some embodiments, the activation domain comprises ten tandem copies of VP16, a VP160 activation domain. In some embodiments, the activation domain comprises p65 activation domain of NFκB. In some embodiments, the activation domain comprises an Epstein-Barr virus R transactivator (Rta) activation domain. In some embodiments, the activation domain comprises a fusion of multiple activators, e.g., a tripartite activator of the VP64, the p65, and the Rta activation domains, (a VPR activation domain). In some embodiments, the activation domain comprises any one of the domains of Table 5 or Table 6, or any homologs or combination thereof.

The DNA demethylation domain in an epigenetic editor may comprise any one of the DNA demethylation proteins known to those skilled in the art and as described herein, or any homologs or combination thereof. In some embodiments, the DNA demethylation domain comprises a TET family protein domain. In some embodiments, the DNA demethylation domain comprises a TET1, TET2, or TET3 protein domain. In some embodiments, the DNA demethylation domain comprises a TET1 protein domain. In some embodiments, the DNA demethylation domain comprises any one of the domains of Table 4, or any homologs or combination thereof.

In some embodiments, an epigenetic editor that can reduce or silence expression of a target gene comprises a Dnmt3A-Dnmt3L fusion protein domain. In some embodiments, the epigenetic editor further comprises a repression scaffold or recruiting protein domain, for example, a KRAB domain, a KAP1 domain, or a MECP2 domain. In some embodiments, the epigenetic editor comprises a Dnmt3A-Dnmt3L fusion domain and an additional repression domain that reduces or silences expression of a target gene. The repression domain in an epigenetic editor may comprise any one of the expression repression proteins known to those skilled in the art and as described herein, or any homologs or combination thereof. In some embodiments, the repression domain comprises a histone deacetylase domain. In some embodiments, the repression domain interacts with a scaffold protein domain that recruits one or more protein domains that repress expression of the target gene. For example, the repression domain may recruit or interact with a scaffold protein domain that recruits a PRMT protein, a HDAC protein, a SETDB1 protein, or a NuRD protein domain. In some embodiments, the repression domain interacts with epigenetically marked DNA nucleotides in a target gene thereby represses or silences expression of the target gene. In some embodiments, the repression domain comprises a MECP2 domain. In some embodiments, the repression domain comprises a KAP1 domain. In some embodiments, the repression domain comprises any one of the domains of Table 2 or Table 3, or any combination or homologs thereof.

In some embodiments, the epigenetic editor comprises a Dnmt3A-Dnmt3L fusion domain and a KAP1 domain. In some embodiments, the epigenetic editor comprises the following configuration: N]-[Dnmt3A-3L]-[KAP1]-[DBD]-[C, wherein the connecting structure]-[ may be any one of the linkers as provided herein. In some embodiments, the epigenetic editor comprises the following configuration: N]-[KAP1]-[Dnmt3A-3L]-[DBD]-[C, wherein the connecting structure]-[ may be any one of the linkers as provided herein. In some embodiments, the epigenetic editor comprises the following configuration: N]-[DBD]-[Dnmt3A-3L]-[KAP1]-[C, wherein the connecting structure]-[ may be any one of the linkers as provided herein. In some embodiments, the epigenetic editor comprises the following configuration: N]-[DBD]-[KAP1]-[Dnmt3A-3L]-[C, wherein the connecting structure]-[ may be any one of the linkers as provided herein. In some embodiments, the epigenetic editor comprises the following configuration: N]-[KAP1]-[DBD]-[Dnmt3A-3L]-[C, wherein the connecting structure]-[ may be any one of the linkers as provided herein. In some embodiments, the epigenetic editor comprises the following configuration: N]-[Dnmt3A-3L]-[DBD]-[KAP1]-[C, wherein the connecting structure]-[ may be any one of the linkers as provided herein.

In some embodiments, the epigenetic editor comprises a Dnmt3A-Dnmt3L fusion domain and a MECP2 domain. In some embodiments, the epigenetic editor comprises the following configuration: N]-[Dnmt3A-3L]-[MECP2]-[DBD]-[C, wherein the connecting structure]-[ may be any one of the linkers as provided herein. In some embodiments, the epigenetic editor comprises the following configuration: N]-[MECP2]-[Dnmt3A-3L]-[DBD]-[C, wherein the connecting structure]-[ may be any one of the linkers as provided herein. In some embodiments, the epigenetic editor comprises the following configuration: N]-[DBD]-[Dnmt3A-3L]-[MECP2]-[C, wherein the connecting structure]-[ may be any one of the linkers as provided herein. In some embodiments, the epigenetic editor comprises the following configuration: N]-[DBD]-[MECP2]-[Dnmt3A-3L]-[C, wherein the connecting structure]-[may be any one of the linkers as provided herein. In some embodiments, the epigenetic editor comprises the following configuration: N]-[MECP2]-[DBD]-[Dnmt3A-3L]-[C, wherein the connecting structure]-[ may be any one of the linkers as provided herein. In some embodiments, the epigenetic editor comprises the following configuration: N]-[Dnmt3A-3L]-[DBD]-[MECP2]-[C, wherein the connecting structure]-[ may be any one of the linkers as provided herein.

In some embodiments, the epigenetic editor comprises a Dnmt3A-Dnmt3L fusion domain and a heterochromatin protein 1 (HP1) domain. In some embodiments, the epigenetic editor comprises the following configuration: N]-[Dnmt3A-3L]-[HP1]-[DBD]-[C, wherein the connecting structure]-[ may be any one of the linkers as provided herein. In some embodiments, the epigenetic editor comprises the following configuration: N]-[HP1]-[Dnmt3A-3L]-[DBD]-[C, wherein the connecting structure]-[ may be any one of the linkers as provided herein. In some embodiments, the epigenetic editor comprises the following configuration: N]-[DBD]-[Dnmt3A-3L]-[HP1]-[C, wherein the connecting structure]-[ may be any one of the linkers as provided herein. In some embodiments, the epigenetic editor comprises the following configuration: N]-[DBD]-[HP1]-[Dnmt3A-3L]-[C, wherein the connecting structure]-[ may be any one of the linkers as provided herein. In some embodiments, the epigenetic editor comprises the following configuration: N]-[HP1]-[DBD]-[Dnmt3A-3L]-[C, wherein the connecting structure]-[ may be any one of the linkers as provided herein. In some embodiments, the epigenetic editor comprises the following configuration: N]-[Dnmt3A-3L]-[DBD]-[HP1]-[C, wherein the connecting structure]-[ may be any one of the linkers as provided herein.

In some embodiments, the epigenetic editor comprises a Dnmt3A-Dnmt3L fusion domain and a SETDB1 domain. In some embodiments, the epigenetic editor comprises the following configuration: N]-[Dnmt3A-3L]-[SETDB1]-[DBD]-[C, wherein the connecting structure]-[ may be any one of the linkers as provided herein. In some embodiments, the epigenetic editor comprises the following configuration: N]-[SETDB1]-[Dnmt3A-3L]-[DBD]-[C, wherein the connecting structure]-[ may be any one of the linkers as provided herein. In some embodiments, the epigenetic editor comprises the following configuration: N]-[DBD]-[Dnmt3A-3L]-[SETDB1]-[C, wherein the connecting structure]-[ may be any one of the linkers as provided herein. In some embodiments, the epigenetic editor comprises the following configuration: N]-[DBD]-[SETDB1]-[Dnmt3A-3L]-[C, wherein the connecting structure]-[may be any one of the linkers as provided herein. In some embodiments, the epigenetic editor comprises the following configuration: N]-[SETDB1]-[DBD]-[Dnmt3A-3L]-[C, wherein the connecting structure]-[ may be any one of the linkers as provided herein. In some embodiments, the epigenetic editor comprises the following configuration: N]-[Dnmt3A-3L]-[DBD]-[SETDB1]-[C, wherein the connecting structure]-[ may be any one of the linkers as provided herein.

In some embodiments, the epigenetic editor comprises a Dnmt3A-Dnmt3L fusion domain and a SETDB1 domain, a KAP1, domain, a KRAB domain, and/or a MECP2 domain, in any order and combination thereof.

In some embodiments, the epigenetic editor that reduces or silences expression of a target gene comprises a DBD and an affinity domain that specifically binds to a repression domain. For example, the epigenetic editor may comprise a DBD and a repression domain antibody. In some embodiments, the epigenetic editor comprises a DBD and a KAP1 affinity domain. In some embodiments, the epigenetic editor comprises a DBD and a KRAB affinity domain. In some embodiments, the epigenetic editor comprises a DBD and a SETDB1 affinity domain. In some embodiments, the epigenetic editor comprises a DBD and a MECP2 affinity domain. In some embodiments, the epigenetic editor comprises a DNA methyltransferase and a repression domain binding affinity domain. In some embodiments, the epigenetic editor comprises a Dnmt3A-Dnm3L fusion and a repression domain binding affinity domain. In some embodiments, the epigenetic editor comprises a Dnmt3A-Dnm3L fusion and KAP1 affinity domain. In some embodiments, the epigenetic editor comprises a Dnmt3A-Dnm3L fusion and KRAB affinity domain. In some embodiments, the epigenetic editor comprises a Dnmt3A-Dnm3L fusion and SETDB1 affinity domain. In some embodiments, the epigenetic editor comprises a Dnmt3A-Dnm3L fusion and MECP2 affinity domain. As used herein, an affinity domain may be an antibody, a single chain antibody, a nanobody, and antigen binding sequence, an antibody, a nanobody, a functional antibody fragment, a single chain variable fragment (scFv), an Fab, a single-domain antibody (sdAb), a VH domain, a VL domain, a VNAR domain, a VHH domain, a bispecific antibody, a diabody, or a functional fragment or a combination thereof.

In some embodiments, the epigenetic editor that reduces or silences expression of a target gene comprises a DBD and an affinity domain that specifically binds to a DNA methyltransferase domain. For example, the epigenetic editor may comprise a DBD and a DNA methyltransferase antibody. In some embodiments, the epigenetic editor comprises a DBD and a Dnmt3A affinity domain. In some embodiments, the epigenetic editor comprises a DBD and a Dnmt3L affinity domain. In some embodiments, the epigenetic editor comprises a repression domain and a DNA methyltransferase binding affinity domain. In some embodiments, the epigenetic editor comprises a repression domain and a Dnmt3A binding affinity domain. In some embodiments, the epigenetic editor comprises a repression domain and Dnmt3L affinity domain. In some embodiments, the epigenetic editor comprises one or more of a KAP1, a KRAB and a MECP2 domain, and a Dnmt3A binding affinity domain. In some embodiments, the epigenetic editor comprises one or more of a KAP1 domain, and a Dnmt3A binding affinity domain. In some embodiments, the epigenetic editor comprises one or more of a KAP1, a KRAB and a MECP2 domain, and a Dnmt3L binding affinity domain. In some embodiments, the epigenetic editor comprises one or more of a KAP1 domain, and a Dnmt3L binding affinity domain. The affinity domain may be an antibody, a single chain antibody, a nanobody, and antigen binding sequence, an antibody, a nanobody, a functional antibody fragment, a single chain variable fragment (scFv), an Fab, a single-domain antibody (sdAb), a VH domain, a VL domain, a VNAR domain, a VHH domain, a bispecific antibody, a diabody, or a functional fragment or a combination thereof.

In some embodiments, the epigenetic editor that reduces or silences expression of a target gene comprises a DBD and a first affinity domain that specifically binds to a DNA methyltransferase domain and a second affinity domain that specifically binds to a repression domain. For example, the epigenetic editor may comprise a DBD and a DNA methyltransferase antibody and a repression domain antibody. In some embodiments, the epigenetic editor comprises a DBD, a KAP1 affinity domain and a Dnmt3A affinity domain. In some embodiments, the epigenetic editor comprises a DBD, a KAP1 affinity domain and a Dnmt3L affinity domain. In some embodiments, the epigenetic editor comprises a DBD, a MECP2 affinity domain and a Dnmt3A affinity domain. In some embodiments, the epigenetic editor comprises a DBD, a MECP2 affinity domain and a Dnmt3L affinity domain. In some embodiments, the epigenetic editor comprises a DBD, a KRAB affinity domain and a Dnmt3A affinity domain. In some embodiments, the epigenetic editor comprises a DBD, a KRAB affinity domain and a Dnmt3L affinity domain. The affinity domain may be an antibody, a single chain antibody, a nanobody, and antigen binding sequence, an antibody, a nanobody, a functional antibody fragment, a single chain variable fragment (scFv), an Fab, a single-domain antibody (sdAb), a VH domain, a VL domain, a VNAR domain, a VHH domain, a bispecific antibody, a diabody, or a functional fragment or a combination thereof.

In some embodiments, an epigenetic editor that can increase expression of a target gene comprises a TET1 protein domain. In some embodiments, the epigenetic editor further comprises a activation protein domain, for example, a VP16 domain, a VP64 domain, a p65 domain or a Rta domain. In some embodiments, the epigenetic editor comprises a VP64-p65-Rta activation domains (a VPR activation domain) and a TET1 domain. In some embodiments, the epigenetic editor comprises the following configuration: N]-[TET1]-[VPR]-[DBD]-[C, wherein the connecting structure]-[ may be any one of the linkers as provided herein. In some embodiments, the epigenetic editor comprises the following configuration: N]-[VPR]-[TET1]-[DBD]-[C, wherein the connecting structure]-[ may be any one of the linkers as provided herein. In some embodiments, the epigenetic editor comprises the following configuration: N]-[DBD]-[TET1]-[VPR]-[C, wherein the connecting structure]-[ may be any one of the linkers as provided herein. In some embodiments, the epigenetic editor comprises the following configuration: N]-[DBD]-[VPR]-[TET1]-[C, wherein the connecting structure]-[ may be any one of the linkers as provided herein. In some embodiments, the epigenetic editor comprises the following configuration: N]-[VPR]-[DBD]-[TET1]-[C, wherein the connecting structure]-[may be any one of the linkers as provided herein. In some embodiments, the epigenetic editor comprises the following configuration: N]-[TET1]-[DBD]-[VPR]-[C, wherein the connecting structure]-[ may be any one of the linkers as provided herein, for example, a peptide linker, an array of epitope tags, or a scaffold nucleic acid (e.g. a RNA that recognizes a MS2 domain fused to the DBD, the TET, or the VPR domain).

In some embodiments, the epigenetic editor that increases expression of a target gene comprises a DBD and an affinity domain that specifically binds to an activation domain. For example, the epigenetic editor may comprise a DBD and an activation domain antibody. In some embodiments, the epigenetic editor comprises a DBD and a TET1 affinity domain. In some embodiments, the epigenetic editor comprises a DBD and a VP16 affinity domain. In some embodiments, the epigenetic editor comprises a DBD and a p65 affinity domain. In some embodiments, the epigenetic editor comprises a DBD and a Rta affinity domain. In some embodiments, the epigenetic editor comprises a DNA demethylase and an activation domain binding affinity domain. In some embodiments, the epigenetic editor comprises a activation domain and a demethylase affinity domain. In some embodiments, the epigenetic editor comprises a DBD and a TET1 affinity domain. In some embodiments, the epigenetic editor comprises a VP16 domain and a TET1 affinity domain. In some embodiments, the epigenetic editor comprises a VP64 domain and a TET1 affinity domain. In some embodiments, the epigenetic editor comprises a Rta domain and a TET1 affinity domain. In some embodiments, the epigenetic editor comprises a p65 domain and a TET1 affinity domain. In some embodiments, the epigenetic editor comprises a VPR activation domain and a TET1 affinity domain. The affinity domain may be an antibody, a single chain antibody, a nanobody, and antigen binding sequence, an antibody, a nanobody, a functional antibody fragment, a single chain variable fragment (scFv), an Fab, a single-domain antibody (sdAb), a VH domain, a VL domain, a VNAR domain, a VHH domain, a bispecific antibody, a diabody, or a functional fragment or a combination thereof.

### Additional Domains

An epigenetic editor system may further comprise an additional heterologous portion or domain (e.g., polynucleotide binding domain such as an RNA or DNA binding protein) that is capable of interacting with, associating with, or capable of forming a complex with a portion or segment (e.g., a polynucleotide motif) of a guide polynucleotide. In some embodiments, the additional heterologous portion or domain (e.g., polynucleotide binding domain such as an RNA or DNA binding protein) can be fused or linked to the DNA binding domain or an effector domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Com coat protein domain, a sterile alpha motif, a telomerase Ku binding motif and Ku protein, a telomerase Sm7 binding motif and Sm7 protein, or any other RNA recognition motif.

### Target Sequences

As used herein, a “target polynucleotide sequence” may be a nucleic acid sequence present in a gene of interest. The target sequence may be in a genome of, or expressed in, a cell. In an aspect, epigenetic editors provided herein are used to bind target polynucleotide sequences and effect epigenetic modifications and/or transcription modulation of the target gene. For example, a target sequence may be recognized by a zinc finger array of an epigenetic editor, or may hybridize with a guide RNA sequence complexed with a nuclease inactive CRISPR protein of an epigenetic editor. In embodiments where the epigenetic editor comprises a gRNA-dCas-effector domain complex, the gRNA is designed to have complementarity to the target sequence (or identity to the opposing strand of the target sequence, e.g. the protospacer sequence). In some embodiments, the gRNA comprises a spacer sequence is 100% identical to a protospacer sequence in the target sequence. In some embodiments, the gRNA sequence comprises a spacer sequence that is about 95%, 90%, 85%, or 80% identical to a protospacer sequence in the target sequence.

In some embodiments, the target sequence is an endogenous sequence of an endogenous gene of a host cell. In some embodiments, the target sequence is an exogenous sequence.

The target sequence may be any region of the polynucleotide (e.g., DNA sequence) suitable for epigenetic editing. For example, the target polynucleotide sequence may be any part of a target gene. In some embodiments, the target polynucleotide sequence is part of a transcriptional regulatory sequence. In some embodiment, the target polynucleotide sequence is part of a promoter, enhancer or silencer. In some embodiments, the target polynucleotide sequence is part of a promoter. In some embodiments, the target polynucleotide sequence is part of an enhancer. In some embodiments, the target polynucleotide sequence is part of a silencer. In some embodiments, the target polynucleotide sequence is within about 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, or 100 base pairs (bp) flanking a transcription start site. In some embodiments, the target polynucleotide sequence is within about 1000, 900, 800, 700, 600, 500, 400, 300, 200, or 100 base pairs (bp) flanking a transcription start site. In some embodiments, the target polynucleotide sequence is within about 500, 400, 300, 200, or 100 base pairs (bp) flanking a transcription start site.

In some embodiments, the target polynucleotide sequence is within about 100 base pairs (bp) flanking a transcription start site.

In some embodiments, the target polynucleotide sequence is a hypomethylated nucleic acid sequence. In some embodiments, the target polynucleotide sequence is a hypermethylated nucleic acid sequence. In some embodiments, the target polynucleotide sequence is at, near, or within a promoter sequence. In some embodiments, the target polynucleotide sequence is at, near, or within a promoter sequence. In aspects, the target polynucleotide sequence is adjacent to a CpG island. In aspects, the target polynucleotide sequence is known to be associated with a disease or condition.

### Modulation of Expression of Target Gene

In some embodiments, the disclosure provides epigenetic editor systems, compositions and methods for epigenetic modifications at a target polynucleotide in a target gene encoding a protein. In some embodiments, the epigenetic editor results in epigenetic modification, e.g. DNA methylation, in a coding region of the target gene, thereby reducing or silencing expression of the target gene. In some embodiments, the epigenetic editor results in epigenetic modification, e.g. DNA methylation, in a regulatory sequence such as a promoter or enhancer of the target gene, thereby reducing or silencing expression of the target gene. In some embodiments, the epigenetic editor results in transcription repression or recruits a transcription repressor to a coding region of the target gene, thereby reducing or silencing expression of the target gene. In some embodiments, the epigenetic editor recruits a transcription repressor to a regulatory sequence such as a promoter or enhancer of the target gene, thereby reducing or silencing expression of the target gene. In some embodiments, the epigenetic editor results in epigenetic modification, e.g. DNA demethylation, in a coding region of the target gene, thereby increasing expression of the target gene. In some embodiments, the epigenetic editor results in epigenetic modification, e.g. DNA demethylation, in a regulatory sequence such as a promoter or enhancer of the target gene, thereby increasing expression of the target gene. In some embodiments, the epigenetic editor results in transcription activation or recruits a transcription activator to a coding region of the target gene, thereby increasing expression of the target gene. In some embodiments, the epigenetic editor recruits a transcription activator to a regulatory sequence such as a promoter or enhancer of the target gene, thereby increasing expression of the target gene.

In some embodiments, the target gene and/or the protein encoded are associated with a disease, disorder, or pathogenic condition.

Epigenetic modifications effected by the epigenetic editors described herein are sequence specific. In some embodiments, the modification is at a specific site of the target polynucleotide. In some embodiments, the modification is at a specific allele of the target gene. Accordingly, the epigenetic modification may result in modulated expression, for example, reduced or increased expression, of one copy of a target gene harboring a specific allele, and not the other copy of the target gene. In some embodiments, the specific allele is associated with a disease, condition, or disorder.

Epigenetic modification may be made at any target genes of a genome of interest, for example, a prokaryote genome, a plant genome, mammalian or human genome. The target gene can be of or derived from any organism and genome thereof. For example, the target gene can be a prokaryotic gene, a eukaryotic gene, an animal gene, a plant gene, a mouse gene, a rat gene, a rabbit gene, a fish gene, an avian gene, a monkey gene, or a human gene. In some embodiments, the target gene is a reporter gene the expression of which can be readily tracked and monitored. Reporter genes and reporter systems include, for example, sequences encoding green fluorescence proteins, red fluorescence proteins, enhanced yellow or enhanced cyan proteins, or luciferase proteins. In some embodiments, the target gene encodes a selectable marker, for example, a beta-galactosidase, a Chloramphenicol acetyltransferase, or a antibiotic resistance marker. In some embodiments, the target gene is associated with, or harbors one or more mutations that are associated with a disease, condition, or disorder. Non-limiting exemplary target genes include HBB, HBA, hMSH2, HMLH1, growth factors GM-SCF, VEGF, EPO, Erb-B2, and hGH.

Target genes also include plant genes for which repression or activation leads to an improvement in plant characteristics, such as improved crop production, disease or herbicide resistance. For example, repression of expression of the FAD2-1 gene results in an advantageous increase in oleic acid and decrease in linoleic and linoleic acids.

In some embodiments, an epigenetic editor provided herein effects an epigenetic modification in a gene that harbors a target sequence. In some embodiments, the epigenetic editor modulates expression of a protein encoded by the gene. In some embodiments, the epigenetic editor reduces the level of a protein encoded by the gene. In some embodiments, the epigenetic editor increases the level of a protein encoded by the gene.

To generate epigenetic edits at a target gene, a target gene polynucleotide may be contacted with the epigenetic editing compositions disclosed herein comprising a target DNA binding domain, an epigenetic effector domain, e.g. an epigenetic repressor domain, wherein the DNA binding domain directs the epigenetic effector domain to a target polynucleotide sequence in the target gene, resulting in the epigenetic modification, e.g., a methylation state modification. In some embodiments, the epigenetic editor effects an alteration in the methylation state of a target DNA sequence in the target gene. In some embodiments, the epigenetic editor effects an alteration in the methylation state of a specific allele in the target gene. In some embodiments, the epigenetic editor effects an alteration in the methylation state of a histone protein associated with the target gene.

In some embodiments, the epigenetic modification reduces transcription of the target gene harboring the target sequence. In some embodiments, the epigenetic modification abolishes transcription of the target gene harboring the target sequence. In some embodiments, the epigenetic modification reduces transcription of a copy of the target gene harboring a specific allele recognized by the epigenetic editor. In some embodiments, the epigenetic modification abolishes transcription of a copy of the target gene harboring a specific allele recognized by the epigenetic editor. In some embodiments, the epigenetic editor reduces the level of a protein encoded by the target gene. In some embodiments, the epigenetic editor eliminates expression of a protein encoded by the target gene. In some embodiments, the epigenetic editor reduces the level of a protein encoded by a copy of the target gene harboring a specific allele recognized by the epigenetic editor. In some embodiments, the epigenetic editor eliminates expression of a protein encoded by a copy of the target gene harboring a specific allele recognized by the epigenetic editor.

In some embodiments, the epigenetic modification increases transcription of the target gene harboring the target sequence. In some embodiments, the epigenetic modification increases transcription of a copy of the target gene harboring a specific allele recognized by the epigenetic editor. In some embodiments, the epigenetic editor increases the level of a protein encoded by the target gene. In some embodiments, the epigenetic editor increases the level of a protein encoded by a copy of the target gene harboring a specific allele recognized by the epigenetic editor.

The target gene may be epigenetically modified in vitro, ex vivo, or in vivo. Accordingly, epigenetic modification of the target gene may modulate expression of a target gene, or an allele thereof, in a cell ex vivo or in a subject in vivo. In some embodiments, the target polynucleotide sequence is the gene locus in the genomic DNA of a cell. In some embodiments, the cell is a cultured cell. In some embodiments, the cell is in vitro. In some embodiments, the cell is ex vivo. In some embodiments, the cell is in vivo. For example, an epigenetic editor, e.g. a fusion protein comprising a zinc finger array and an effector domain, or a sgRNA complexed with a Cas protein-effector domain fusion, may be expressed in a cell where modulated expression of a target gene is desired to thereby allow contact of the target gene with the epigenetic editor described herein. In some embodiments, the cell is from a mammal. In some embodiments, the mammal is a human. In some embodiments, the mammal is a rodent. In some embodiments, the rodent is a mouse. In some embodiments, the rodent is a rat.

In some embodiments, the epigenetic editors described herein reduces expression of a target gene by at least about 20%, at least about 30%, at least about 40%, at least about 50%, at least about 60%, at least about 70%, at least about 80%, at least about 90%, at least about 95%, at least about 99%, or more, as measured by transcription of the target gene in a cell, a tissue, or a subject as compared to a control cell, control tissue, or a control subject. In some embodiments, the epigenetic editors described herein reduces expression of a copy of target gene by at least about 20%, at least about 30%, at least about 40%, at least about 50%, at least about 60%, at least about 70%, at least about 80%, at least about 90%, at least about 95%, at least about 99%, or more, as measured by transcription of the copy of the target gene in a cell, a tissue, or a subject as compared to a control cell, control tissue, or a control subject. In some embodiments, the copy of the target gene harbors a specific sequence or allele recognized by the epigenetic editor. In some embodiments, the epigenetically modified copy encodes a functional protein. Accordingly, in some embodiments, an epigenetic editor composition disclosed herein reduces or abolishes expression and/or function of protein encoded by a target gene, by reducing or abolishing expression of a functional protein encoded by the target gene. For example, the methods and composition disclosed herein may reduce expression and/or function of a protein encoded by the target gene by at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold, at least 60-fold, at least 70-fold, at least 80-fold, at least 90-fold, or at least 100 fold in a cell, a tissue, or a subject as compared to a control cell, control tissue, or a control subject.

In some embodiments, the epigenetic editors described herein increases expression of a target gene by at least about 20%, at least about 30%, at least about 40%, at least about 50%, at least about 60%, at least about 70%, at least about 80%, at least about 90%, at least about 95%, at least about 99%, at least 100%, at least 110%, at least 120%, at least 130%, at least 140%, at least 150%, at least 160%, at least 170%, at least 180%, at least 190%, at least 200%, at least 250%, at least 300%, at least 350%, at least 400%, at least 450%, at least 500% or more, as measured by transcription of the target gene in a cell, a tissue, or a subject as compared to a control cell, control tissue, or a control subject. In some embodiments, the epigenetic editors described herein increases expression of a copy of target gene by at least about 20%, at least about 30%, at least about 40%, at least about 50%, at least about 60%, at least about 70%, at least about 80%, at least about 90%, at least about 95%, at least about 99%, at least 100%, at least 110%, at least 120%, at least 130%, at least 140%, at least 150%, at least 160%, at least 170%, at least 180%, at least 190%, at least 200%, at least 250%, at least 300%, at least 350%, at least 400%, at least 450%, at least 500% or more, as measured by transcription of the copy of the target gene in a cell, a tissue, or a subject as compared to a control cell, control tissue, or a control subject. In some embodiments, the copy of the target gene harbors a specific sequence or allele recognized by the epigenetic editor. In some embodiments, the epigenetically modified copy encodes a functional protein. Accordingly, in some embodiments, an epigenetic editor composition disclosed herein increases expression and/or function of protein encoded by a target gene, by increasing expression of a functional protein encoded by the target gene. For example, the methods and composition disclosed herein may increase expression and/or function of a protein encoded by the target gene by at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold, at least 30-fold, at least 35-fold, at least 40-fold, at least 45-fold, at least 50-fold, at least 60-fold, at least 70-fold, at least 80-fold, at least 90-fold, or at least 100 fold in a cell, a tissue, or a subject as compared to a control cell, control tissue, or a control subject.

Methods for determining the expression level of a gene, for example the target of an epigenetic editor, are known in the art. For example, transcript level of a gene may be determined by reverse transcription PCR, quantitative RT-PCR, droplet digital PCR (ddPCR), Northern blot, RNA sequencing, DNA sequencing (e.g., sequencing of complementary deoxyribonucleic acid (cDNA) obtained from RNA); next generation (Next-Gen) sequencing, nanopore sequencing, pyrosequencing, or Nanostring sequencing. Protein level expressed from a gene may be determined by western blotting, enzyme linked immuno-absorbance assays, mass-spectrometry, immunohistochemistry, or flow cytometry analysis. Gene expression product levels may be normalized to an internal standard such as total messenger ribonucleic acid (mRNA) or the expression level of a particular gene, e.g., a house keeping gene.

In some embodiments, the effect of an epigenetic editor in modulating target gene expression may be examined using a reporter system. For example, an epigenetic editor may be designed to target a reporter gene encoding a reporter protein, e.g. a fluorescent protein. Expression of the reporter gene in such a model system may be monitored by, e.g., flow cytometry, fluorescence-activated cell sorting (FACS), or fluorescence microscopy. In some embodiments, a population of cells may be transfected with a vector which harbors a reporter gene. The vector may be constructed such that the reporter gene is expressed when the vector transfects a cell. Suitable reporter genes include genes encoding fluorescent proteins, for example green, yellow, cherry, cyan or orange fluorescent proteins. The population of cells carrying the reporter system may be transfected with DNA, mRNA, or vectors encoding the epigenetic editor targeting the reporter gene. The level of expression of the reporter gene may be quantified using a suitable technique, such as FACS.

Epigenetic editors described herein may be expressed in a host cell transiently, or may be integrated in a genome of the host cell. Both transiently expressed and integrated epigenetic editors can effect stable epigenetic modifications. For example, after introduction of an epigenetic editor comprising a DNA binding domain specific for a target gene and an epigenetic repression domain to a host cell, the target gene in the host cell may be stably or permanently repressed. In some embodiments, expression of the target gene is reduced for at least 1 week, at least 2 weeks, at least 3 weeks, at least 4 weeks, at least 5 weeks, at least 6 weeks, at least 7 weeks, at least 2 months, at least 3 months, at least 5 months, at least 6 months, at least 1 year, at least 2 years, or for the entire lifetime of the cell or the subject carrying the cell, as compared to the level of expression in the absence of the epigenetic editor. In some embodiments, expression of the target gene is silenced for at least 1 week, at least 2 weeks, at least 3 weeks, at least 4 weeks, at least 5 weeks, at least 6 weeks, at least 7 weeks, at least 2 months, at least 3 months, at least 5 months, at least 6 months, at least 1 year, at least 2 years, or for the entire lifetime of the cell or the subject carrying the cell as compared to the level of expression in the absence of the epigenetic editor. In some embodiments, after introduction of an epigenetic editor comprising a DNA binding domain specific for a target gene and an epigenetic activation domain to a host cell, the target gene in the host cell is stably or permanently activated. In some embodiments, expression of the target gene is increased for at least 1 week, at least 2 weeks, at least 3 weeks, at least 4 weeks, at least 5 weeks, at least 6 weeks, at least 7 weeks, at least 2 months, at least 3 months, at least 5 months, at least 6 months, at least 1 year, at least 2 years, or for the entire lifetime of the cell or the subject carrying the cell as compared to the level of expression in the absence of the epigenetic editor.

The epigenetic modification described herein may be inherited by the progeny of host cells that are contacted or introduced with an epigenetic editor. For example, in some embodiments, after introduction of an epigenetic editor comprising a DNA binding domain specific for a target gene and an epigenetic repression domain to a stem cell, e.g., a hematopoietic stem cell, expression of the target gene is also repressed in cells differentiated from the stem cell compared to cells differentiated from a control stem cell in the absence of the epigenetic editor. In some embodiments, expression of the target gene is silenced in cells differentiated from the stem cell. In some embodiments, after introduction of an epigenetic editor comprising a DNA binding domain specific for a target gene and an epigenetic activation domain to a stem cell, e.g., a hematopoietic stem cell, expression of the target gene is also increased in cells differentiated from the stem cell compared to cells differentiated from a control stem cell in the absence of the epigenetic editor.

Modulation of target gene expression can be assayed by determining any parameter that is indirectly or directly affected by the expression of the target gene. Such parameters include, e.g., changes in RNA or protein levels; changes in protein activity; changes in product levels; changes in downstream gene expression; changes in transcription or activity of reporter genes such as, for example, luciferase, CAT, beta-galactosidase, or GFP; changes in signal transduction; changes in phosphorylation and dephosphorylation; changes in receptor-ligand interactions; changes in concentrations of second messengers such as, for example, cGMP, cAMP, IP3, and Ca2+; changes in cell growth, changes in neovascularization, and/or changes in any functional effect of gene expression. Measurements can be made in vitro, in vivo, and/or ex vivo. Such functional effects can be measured by conventional methods, e.g., measurement of RNA or protein levels, measurement of RNA stability, and/or identification of downstream or reporter gene expression. Readout can be by way of, for example, chemiluminescence, fluorescence, colorimetric reactions, antibody binding, inducible markers, ligand binding assays; changes in intracellular second messengers such as cGMP and inositol triphosphate (IP3); changes in intracellular calcium levels; cytokine release, and the like.

To determine the level of gene expression modulation by a ZFP, cells contacted with ZFPs are compared to control cells, e.g., without the zinc finger protein or with a non-specific ZFP, to examine the extent of inhibition or activation. Control samples are assigned a relative gene expression activity value of 100%. Modulation/inhibition of gene expression is achieved when the gene expression activity value relative to the control is about 80%, preferably 50% (i.e., 0.5× the activity of the control), more preferably 25%, more preferably 5-0%. Modulation/activation of gene expression is achieved when the gene expression activity value relative to the control is 110%, more preferably 150% (i.e., 1.5× the activity of the control), more preferably 200-500%, more preferably 1000-2000% or more.

### Delivery

In an aspect, provided herein is a composition for gene expression modulation comprising the epigenetic editor as provided herein that generates epigenetic modifications at target genes. The epigenetic editor, or nucleic acid encoding the epigenetic editor or components thereof (e.g. nucleic acids encoding an epigenetic editor fusion protein comprising a zinc finger—repressor fusion, a Cas9-repressor fusion, and or nucleic acids encoding one or more guide RNAs) may be introduced to a cell via various ways known in the art. For example, in some embodiments, the epigenetic editor is delivered to a host cell or integrated into the genome of the host cell, or for transient expression in the host cell.

In some embodiments, the nucleic acid encoding the epigenetic editor or components thereof is operatively linked to a promoter and/or a regulatory sequence. The term “operably linked,” as used herein, means that the nucleotide sequence of interest is linked to regulatory sequence(s) in a manner that allows for expression of the nucleotide sequence. The term “regulatory sequence,” as used herein, includes, but is not limited to promoters, enhancers and other expression control elements. Such regulatory sequences are well known in the art and are described, for example, in Goeddel; Gene Expression Technology: Methods in Enzymology 185, Academic Press, San Diego, CA (1990).

In some embodiments, the composition further comprises a vector that comprises the nucleic acid sequence encoding an epigenetic editor protein. In some embodiments, the vector may be an expression vector. In some embodiments, the vector is a plasmid or a viral vector. The term “vector,” as used herein, refers to a nucleic acid molecule capable of transporting another nucleic acid to which it has been linked. In some examples, a vector is an expression vector that is capable of directing the expression of nucleic acids to which they are operatively linked. Examples of expression vectors include, but are not limited to, plasmid vectors, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors.

Non-viral delivery systems include but are not limited to DNA transfection methods. Here, transfection includes a process using a non-viral vector to deliver a gene to a target cell. Typical transfection methods include electroporation, DNA biolistics, lipid-mediated transfection, compacted DNA-mediated transfection, liposomes, immunoliposomes, lipofection, cationic agent-mediated transfection, cationic facial amphiphiles (CFAs).

In some embodiments, the epigenetic editor is delivered to a host cell for transient expression, e.g., via a transient expression vector. Transient expression of a epigenetic editor may result in prolonged or permanent epigenetic modification of the target gene. For example, the epigenetic modification may be stable for at least 1, 2, 3, 4, 5, 6, 7, 8, 9, 11, 12 weeks, 3, 4, 5, 6, 7, 8, 9, 10, 11, 12 months or more after introduction of the epigenetic editor into the host cell. The epigenetic modification may be maintained after one or more mitotic events of the host cell. The epigenetic modification may be maintained after one or more meiotic events of the host cell. In some embodiments, the epigenetic modification is maintained across generations in offspring generated or derived from the host cell.

In some embodiments, a nucleic acid sequence encoding an epigenetic editor or components thereof is a DNA, an RNA or mRNA, or a modified nucleic acid sequence. For example, a mRNA sequence encoding an epigenetic editor fusion protein may be chemically modified, or may comprise a 5′Cap, or one or more 3′ modifications.

Nucleic acids encoding epigenetic editors can be delivered directly to cells as naked DNA or RNA, for instance by means of transfection or electroporation, or can be conjugated to molecules (e.g., N-acetylgalactosamine) promoting uptake by the target cells. Nucleic acid vectors, such as the vectors can also be used. In particular embodiments, a polynucleotide, e.g. a mRNA encoding an epigenetic editor or a functional component thereof may be co-electroporated with a combination of multiple guide RNAs as described herein.

Nucleic acid vectors can comprise one or more sequences encoding a domain of a fusion protein or an epigenetic editor as described herein. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vectors can include a Cas9 coding sequence that includes one or more nuclear localization sequences (e.g., a nuclear localization sequence from SV40), and one or more effector domains such as repression domains.

In particular embodiments, a fusion protein, a protein domain, or a whole or a part of epigenetic editor components is encoded by a polynucleotide present in a viral vector (e.g., adeno-associated virus (AAV), AAV3, AAV3b, AAV4, AAV5, AAV6, AAV7, AAV8, AAV9, AAVrh8, AAV10, and variants thereof), or a suitable capsid protein of any viral vector. Thus, in some aspects, the disclosure relates to the viral delivery of a fusion protein. Examples of viral vectors include retroviral vectors (e.g. Maloney murine leukemia virus, MML-V), adenoviral vectors (e.g. AD100), lentiviral vectors (HIV and FIV-based vectors), herpesvirus vectors (e.g. HSV-2).

In some embodiments, an epigenetic editor protein is encoded by a polynucleotide present in an adeno-associated virus (AAV) vector. In some embodiments, the epigenetic editor protein comprises a zinc finger array in the DNA binding domain. Without wishing to be bound by any theory, epigenetic editors using zinc finger array instead of larger DNA binding domains such as Cas protein domains can be conveniently packed in viral vectors, e.g. AAV vector, given the small size of zinc fingers. In some embodiments, the polynucleotide encoding the epigenetic editor is of length of about 1000 bp, 1.1 kilobases (kb), 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, or less. In some embodiments, The polynucleotide encoding the epigenetic editor is of length of about 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, 4.1 kb, 4.2 kb, 4.3 kb, 4.4 kb, 4.5 kb, 4.6 kb, 4.7 kb, 4.8 kb, 4.9 kb, 5 kb or less.

Any AAV serotype, e.g., human AAV serotype, can be used including, but not limited to, AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), AAV serotype 11 (AAV11), a variant thereof, or a shuffled variant thereof (e.g., a chimeric variant thereof). In some embodiments, an AAV variant has at least 90%, e.g., 90%, 91%, 92%, 93%, 94%, 95%, 96%, 97%, 98%, 99% or more amino acid sequence identity to a wild-type AAV. An AAV1 variant can have at least 90%, e.g., 90%, 91%, 92%, 93%, 94%, 95%, 96%, 97%, 98%, 99% or more amino acid sequence identity to a wild-type AAV1. An AAV2 variant can have at least 90%, e.g., 90%, 91%, 92%, 93%, 94%, 95%, 96%, 97%, 98%, 99% or more amino acid sequence identity to a wild-type AAV2. An AAV3 variant can have at least 90%, e.g., 90%, 91%, 92%, 93%, 94%, 95%, 96%, 97%, 98%, 99% or more amino acid sequence identity to a wild-type AAV3. An AAV4 variant can have at least 90%, e.g., 90%, 91%, 92%, 93%, 94%, 95%, 96%, 97%, 98%, 99% or more amino acid sequence identity to a wild-type AAV4. An AAV5 variant can have at least 90%, e.g., 90%, 91%, 92%, 93%, 94%, 95%, 96%, 97%, 98%, 99% or more amino acid sequence identity to a wild-type AAV5. An AAV6 variant can have at least 90%, e.g., 90%, 91%, 92%, 93%, 94%, 95%, 96%, 97%, 98%, 99% or more amino acid sequence identity to a wild-type AAV6. An AAV7 variant can have at least 90%, e.g., 90%, 91%, 92%, 93%, 94%, 95%, 96%, 97%, 98%, 99% or more amino acid sequence identity to a wild-type AAV7. An AAV8 variant can have at least 90%, e.g., 90%, 91%, 92%, 93%, 94%, 95%, 96%, 97%, 98%, 99% or more amino acid sequence identity to a wild-type AAV8. An AAV9 variant can have at least 90%, e.g., 90%, 91%, 92%, 93%, 94%, 95%, 96%, 97%, 98%, 99% or more amino acid sequence identity to a wild-type AAV9. An AAV10 variant can have at least 90%, e.g., 90%, 91%, 92%, 93%, 94%, 95%, 96%, 97%, 98%, 99% or more amino acid sequence identity to a wild-type AAV10. An AAV11 variant can have at least 90%, e.g., 90%, 91%, 92%, 93%, 94%, 95%, 96%, 97%, 98%, 99% or more amino acid sequence identity to a wild-type AAV11. An AAV12 variant can have at least 90%, e.g., 90%, 91%, 92%, 93%, 94%, 95%, 96%, 97%, 98%, 99% or more amino acid sequence identity to a wild-type AAV12.

In some instances, one or more regions of at least two different AAV serotype viruses are shuffled and reassembled to generate an AAV chimera virus. For example, a chimeric AAV can comprise inverted terminal repeats (ITRs) that are of a heterologous serotype compared to the serotype of the capsid. The resulting chimeric AAV virus can have a different antigenic reactivity or recognition, compared to its parental serotypes. In some embodiments, a chimeric variant of an AAV includes amino acid sequences from 2, 3, 4, 5, or more different AAV serotypes.

Descriptions of AAV variants and methods for generating thereof are found, e.g., in Weitzman and Linden. Chapter 1-Adeno-Associated Virus Biology in Adeno-Associated Virus: Methods and Protocols Methods in Molecular Biology, vol. 807. Snyder and Moullier, eds., Springer, 2011; Potter et al., Molecular Therapy-Methods & Clinical Development, 2014, 1, 14034; Bartel et al., Gene Therapy, 2012, 19, 694-700; Ward and Walsh, Virology, 2009, 386(2):237-248; and Li et al., Mol Ther, 2008, 16(7):1252-1260, each incorporated herein by reference in its entirety. AAV virions (e.g., viral vectors or viral particle) described herein can be transduced into cells to introduce the epigenetic editor or any component thereof into the cell. An epigenetic editor can be packaged into an AAV viral vector according to any method known to those skilled in the art. Examples of useful methods are described in McClure et al., J Vis Exp, 2001, 57:3378.

A nucleic acid vector described herein can also include any suitable number of regulatory/control elements, e.g., promoters, enhancers, introns, polyadenylation signals, Kozak consensus sequences, or internal ribosome entry sites (IRES). These elements are well known in the art.

Nucleic acid vectors according to this disclosure include recombinant viral vectors. Exemplary viral vectors are set forth herein above. Other viral vectors known in the art can also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which can be organic or inorganic. Nanoparticles are well known in the art. Any suitable nanoparticle design can be used to deliver genome editing system components or nucleic acids encoding such components. For instance, organic (e.g. lipid and/or polymer) nanoparticles can be suitable for use as delivery vehicles in certain embodiments of this disclosure.

### Method of Treatment

Also provided herein are methods for treating or preventing a condition in a subject in need thereof, the method comprising administering to the subject the epigenetic editor composition as described herein, wherein the epigenetic editor complex or protein effects an epigenetic modification of a target polynucleotide in a target gene associated with a disease, condition or disorder in a subject and modulates expression of the target, thereby treating or preventing the disease, condition or disorder.

Epigenetic modifications effected by the epigenetic editors described herein are sequence specific. In some embodiments, the modification is at a specific site of the target polynucleotide. In some embodiments, the modification is at a specific allele of the target gene. Accordingly, the epigenetic modification may result in modulated expression, for example, reduced or increased expression, of one copy of a target gene harboring a specific allele, and not the other copy of the target gene. In some embodiments, the specific allele is associated with a disease, condition, or disorder.

In some embodiments, the epigenetic editor reduces expression of a target gene associated with a disease, condition or disorder.

Epigenetic editors described herein may be administered to a subject in need thereof, in a therapeutically effective amount, to treat a disease, condition or disorder.

In another aspect, provided herein is a method for treating or preventing a condition in a subject in need thereof, the method comprising administering to the subject the epigenetic editing complex, vectors, nucleic acids, proteins, or compositions as provided herein, wherein the nucleic acid binding domain of the epigenetic editor directs the effector domain to generate an epigenetic modification in a target polynucleotide sequence in a cell of the subject, thereby modulating expression of the target gene and treating or preventing the condition.

In some embodiments, the modification reduces expression of a functional protein encoded by the target gene in the subject.

A patient who is being treated for a condition, a disease or a disorder is one who a medical practitioner has diagnosed as having such a condition. Diagnosis may be by any suitable means. Diagnosis and monitoring may involve, for example, detecting the presence of diseased, dying or dead cells in a biological sample (e.g., tissue biopsy, blood test, or urine test), detecting the presence of plaques, detecting the level of a surrogate marker in a biological sample, or detecting symptoms associated with a condition. A patient in whom the development of a condition is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

A subject may have a disease, a symptom of the disease, or a predisposition toward the disease, with the purpose to cure, heal, alleviate, relieve, alter, remedy, ameliorate, improve, or affect the disease, the symptom of the disease, or the predisposition toward the disease. In some embodiments, the subject has hypercholesterolemia. In some embodiments, the subject has atherosclerotic vascular disease. In some embodiments, the subject has hypertriglyceridemia. In some embodiments, the subject has diabetes. In some embodiments, the subject is a mammal. In some embodiments, the subject is a non-human primate. In some embodiments, the subject is human. Alleviating a disease includes delaying the development or progression of the disease, or reducing disease severity. Alleviating the disease does not necessarily require curative results.

As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset.

As used herein “onset” or “occurrence” of a disease includes initial onset and/or recurrence. Conventional methods, known to those of ordinary skill in the art of medicine, can be used to administer the isolated polypeptide or pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir.

The therapeutic methods of the disclosure may be carried out on subjects displaying pathology resulting from a disease or a condition, subjects suspected of displaying pathology resulting from a disease or a condition, and subjects at risk of displaying pathology resulting from a disease or a condition. For example, subjects that have a genetic predisposition to a disease or a condition can be treated prophylactically. Subjects exhibiting symptoms associated with a condition, a disease or a disorder may be treated to decrease the symptoms or to slow down or prevent further progression of the symptoms. The physical changes associated with the increasing severity of a disease or a condition are shown herein to be progressive. Thus, in embodiments of the disclosure, subjects exhibiting mild signs of the pathology associated with a condition or a disease may be treated to improve the symptoms and/or prevent further progression of the symptoms.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Adjustment and manipulation of established dosages (e.g., frequency and duration) are well within the ability of those skilled in the art. The treatment, such as those disclosed herein, can be administered to the subject on a daily, twice daily, biweekly, monthly or any applicable basis that is therapeutically effective. In embodiments, the treatment is only on an as-needed basis, e.g., upon appearance of signs or symptoms of a condition or a disease.

Toxicity and therapeutic efficacy of the compositions of the disclosure can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects (the ratio LD50/ED50) is the therapeutic index. Agents that exhibit high therapeutic indices are preferred. The dosage of agents lies preferably within a range of circulating concentrations that include the ED50 with little or no toxicity. While agents that exhibit toxic side effects may be used, care should be taken to design a delivery system that targets such agents to the site of affected tissue in order to minimize potential damage to uninfected cells and, thereby, reduce side effects.

The skilled artisan will appreciate that certain factors may influence the dosage and frequency of administration required to effectively treat a subject, including but not limited to the severity of the disease or disorder, previous treatments, the general characteristics of the subject including health, sex, weight and/or age of the subject, and other diseases present. Moreover, treatment of a subject with a therapeutically effective amount of the compositions can include a single treatment or, preferably, can include a series of treatments. It will also be appreciated that the effective dosage of the composition of the disclosure used for treatment may increase or decrease over the course of a particular treatment. Changes in dosage may result and become apparent from the results of diagnostic assays as described herein. The therapeutically-effective dosage will generally be dependent on the patient's status at the time of administration. The precise amount can be determined by routine experimentation but may ultimately lie with the judgment of the clinician, for example, by monitoring the patient for signs of disease and adjusting the treatment accordingly.

Frequency of administration may be determined and adjusted over the course of therapy, and is generally, but not necessarily, based on treatment and/or suppression and/or amelioration and/or delay of a disease. Alternatively, sustained continuous release formulations of a polypeptide or a polynucleotide may be appropriate. Various formulations and devices for achieving sustained release are known in the art. In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays.

The dosing regimen (including a composition disclosed herein) can vary over time. In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the polypeptide or the polynucleotide (such as the half-life of the polypeptide or the polynucleotide, and other considerations well known in the art).

For the purpose of the present disclosure, the appropriate therapeutic dosage of a composition as described herein will depend on the specific agent (or compositions thereof) employed, the formulation and route of administration, the type and severity of the disease, whether the polypeptide or the polynucleotide is administered for preventive or therapeutic purposes, previous therapy, the subject's clinical history and response to the antagonist, and the discretion of the attending physician. Typically, the clinician will administer a polypeptide until a dosage is reached that achieves the desired result.

Administration of one or more compositions can be continuous or intermittent, depending, for example, upon the recipient's physiological condition, whether the purpose of the administration is therapeutic or prophylactic, and other factors known to skilled practitioners. The administration of a composition may be essentially continuous over a preselected period of time or may be in a series of spaced dose, e.g., either before, during, or after developing a disease.

The methods and compositions of the disclosure described herein including embodiments thereof can be administered with one or more additional therapeutic regimens or agents or treatments, which can be co-administered to the mammal. By “co-administering” is meant administering one or more additional therapeutic regimens or agents or treatments and the composition of the disclosure sufficiently close in time to enhance the effect of one or more additional therapeutic agents, or vice versa. In this regard, the composition of the disclosure described herein can be administered simultaneously with one or more additional therapeutic regimens or agents or treatments, at a different time, or on an entirely different therapeutic schedule (e.g., the first treatment can be daily, while the additional treatment is weekly). For example, in embodiments, the secondary therapeutic regimens or agents or treatments are administered simultaneously, prior to, or subsequent to the composition of the disclosure.

### Pharmaceutical Compositions

In some aspects, provided herein, is a pharmaceutical composition for epigenetic modification comprising an epigenetic editor or epigenetic editor complex described herein, or one or more nucleic acid sequences encoding components of the epigenetic editor complex, e.g., nucleic acids encoding an epigenetic editor fusion protein and/or a guide RNA, and a pharmaceutically acceptable carrier. The composition for epigenetic modification described herein can be formulated into pharmaceutical compositions. Pharmaceutical compositions are formulated in a conventional manner using one or more pharmaceutically acceptable inactive ingredients that facilitate processing of the active compounds into preparations that can be used pharmaceutically. Suitable formulations for use in the present disclosure and methods of delivery are generally well known in the art. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition can be a mixture of an epigenetic editor or nucleic acids encoding same as described herein and one or more other chemical components (i.e., pharmaceutically acceptable ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the epigenetic editor, for example, a nucleic acid encoding a zinc finger-epigenetic effector fusion protein or a Cas9-epigenetic effector fusion protein and a gRNA or sgRNA described herein to an organism or a subject in need thereof.

The pharmaceutical compositions of the present disclosure can be administered to a subject using any suitable methods known in the art. The pharmaceutical compositions described herein can be administered to the subject in a variety of ways, including parenterally, intravenously, intradermally, intramuscularly, colonically, rectally, or intraperitoneally. In some embodiments, the pharmaceutical compositions can be administered by intraperitoneal injection, intramuscular injection, subcutaneous injection, or intravenous injection of the subject. In some embodiments, the pharmaceutical compositions can be administered parenterally, intravenously, intramuscularly, or orally.

For administration by inhalation, the adenovirus described herein can be formulated for use as an aerosol, a mist, or a powder. For buccal or sublingual administration, the pharmaceutical compositions may be formulated in the form of tablets, lozenges, or gels formulated in a conventional manner. In some embodiments, the adenovirus described herein can be prepared as transdermal dosage forms. In some embodiments, the adenovirus described herein can be formulated into a pharmaceutical composition suitable for intramuscular, subcutaneous, or intravenous injection. In some embodiments, the adenovirus described herein can be administered topically and can be formulated into a variety of topically administrable compositions, such as solutions, suspensions, lotions, gels, pastes, medicated sticks, balms, creams, or ointments. In some embodiments, the adenovirus described herein can be formulated in rectal compositions such as enemas, rectal gels, rectal foams, rectal aerosols, suppositories, jelly suppositories, or retention enemas. In some embodiments, the adenovirus described herein can be formulated for oral administration such as a tablet, a capsule, or liquid in the form of aqueous suspensions or solutions selected from the group including, but not limited to, aqueous oral dispersions, emulsions, solutions, elixirs, gels, and syrups.

In some embodiments, the pharmaceutical composition for epigenetic modification comprising an epigenetic editor described herein or nucleic acid sequences encoding the same further comprises a therapeutic agent. The additional therapeutic agent may modulate different aspects of the disease, disorder, or condition being treated and provide a greater overall benefit than administration of either the replication competent recombinant adenovirus or the therapeutic agent alone. Therapeutic agents include, but are not limited to, a chemotherapeutic agent, a radiotherapeutic agent, a hormonal therapeutic agent, and/or an immunotherapeutic agent. In some embodiments, the therapeutic agent may be a radiotherapeutic agent. In some embodiments, the therapeutic agent may be a hormonal therapeutic agent. In some embodiments, the therapeutic agent may be an immunotherapeutic agent. In some embodiments, the therapeutic agent is a chemotherapeutic agent. Preparation and dosing schedules for additional therapeutic agents can be used according to manufacturers' instructions or as determined empirically by a skilled practitioner. For example, preparation and dosing schedules for chemotherapy are also described in The Chemotherapy Source Book, 4th Edition, 2008, M. C. Perry, Editor, Lippincott, Williams & Wilkins, Philadelphia, PA.

The subjects that can be treated with epigenetic modification compositions can be any subject with a disease or a condition. For example, the subject may be a eukaryotic subject, such as an animal. In some embodiments, the subject is a mammal, e.g., human. In some embodiments, the subject is a human. In some embodiments, the subject is a non-human animal. In some embodiments, the subject is a fetus, an embryo, or a child. In some embodiments, the subject is a non-human primate such as chimpanzee, and other apes and monkey species; farm animals such as cattle, horses, sheep, goats, pigs; domestic animals such as rabbits, dogs, and cats; laboratory animals including rodents, such as rats, mice, and guinea pigs, and the like.

In some embodiments, the subject is prenatal (e.g., a fetus), a child (e.g., a neonate, an infant, a toddler, a preadolescent), an adolescent, a pubescent, or an adult (e.g., an early adult, a middle-aged adult, a senior citizen). The human subject can be between about 0 month and about 120 years old, or older. The human subject can be between about 0 and about 12 months old; for example, about 1, 2, 3, 4, 5, 6, 7, 8, 9, 10, 11, or 12 months old. The human subject can be between about 0 and 12 years old; for example, between about 0 and 30 days old; between about 1 month and 12 months old; between about 1 year and 3 years old; between about 4 years and 5 years old; between about 4 years and 12 years old; about 1, 2, 3, 4, 5, 6, 7, 8, 9, 10, 11, or 12 years old. The human subject can be between about 13 years and 19 years old; for example, about 13, 14, 15, 16, 17, 18, or 19 years old. The human subject can be between about 20 and about 39 years old; for example, about 20, 21, 22, 23, 24, 25, 26, 27, 28, 29, 30, 31, 32, 33, 34, 35, 36, 37, 38, or 39 years old. The human subject can be between about 40 to about 59 years old; for example, about 40, 41, 42, 43, 44, 45, 46, 47, 48, 49, 50, 51, 52, 53, 54, 55, 56, 57, 58, or 59 years old. The human subject can be greater than 59 years old; for example, about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 years old. The human subjects can include male subjects and/or female subjects.

In another aspect, provided herein is a lipid nanoparticle (LNP) comprising the composition as provided herein. As used herein, a “lipid nanoparticle (LNP) composition” or a “nanoparticle composition” is a composition comprising one or more described lipids. LNP compositions are typically sized on the order of micrometers or smaller and may include a lipid bilayer. Nanoparticle compositions encompass lipid nanoparticles (LNPs), liposomes (e.g., lipid vesicles), and lipoplexes. In some embodiments, a LNP refers to any particle that has a diameter of less than 1000 nm, 500 nm, 250 nm, 200 nm, 150 nm, 100 nm, 75 nm, 50 nm, or 25 nm. In some embodiments, a nanoparticle may range in size from 1-1000 nm, 1-500 nm, 1-250 nm, 25-200 nm, 25-100 nm, 35-75 nm, or 25-60 nm.

In some embodiments, an LNP may be made from cationic, anionic, or neutral lipids. In some embodiments, an LNP may comprise neutral lipids, such as the fusogenic phospholipid 1,2-Dioleoyl-sn-glycero-3-phosphoethanolamine (DOPE) or the membrane component cholesterol, as helper lipids to enhance transfection activity and nanoparticle stability. In some embodiments, an LNP may comprise hydrophobic lipids, hydrophilic lipids, or both hydrophobic and hydrophilic lipids. Any lipid or combination of lipids that are known in the art can be used to produce an LNP. Examples of lipids used to produce LNPs include, but are not limited to DOTMA (N[1-(2,3-dioleyloxy)propyl]-N,N,N-trimethylammonium chloride), DOSPA (N,N-dimethyl-N-([2-sperminecarboxamido]ethyl)-2,3-bis(dioleyloxy)-1-propaniminium pentahydrochloride), DOTAP (1,2-Dioleoyl-3-trimethylammonium propane), DMRIE (N-(2-hydroxyethyl)-N,N-dimethyl-2,3-bis(tetradecyloxy-1-propanaminiumbromide), DC-cholesterol (3β-[N-(N′,N′-dimethylaminoethane)-carbamoyl]cholesterol), DOTAP-cholesterol, GAP-DMORIE-DPyPE, and GL67A-DOPE-DMPE (,2-Bis(dimethylphosphino)ethane)-polyethylene glycol (PEG). Examples of cationic lipids include, but are not limited to, 98N12-5, C12-200, DLin-KC2-DMA (KC2), DLin-MC3-DMA (MC3), XTC, MD1, and 7C1. Examples of neutral lipids include, but are not limited to, DPSC, DPPC (Dipalmitoylphosphatidylcholine), POPC (1-palmitoyl-2-oleoyl-sn-glycero-3-phosphocholine), DOPE, and SM (sphingomyelin). Examples of PEG-modified lipids include, but are not limited to, PEG-DMG (Dimyristoyl glycerol), PEG-CerC14, and PEG-CerC20. In some embodiments, the lipids may be combined in any number of molar ratios to produce a LNP. In some embodiments, the polynucleotide may be combined with lipid(s) in a wide range of molar ratios to produce an LNP.

Also disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, and any packaging material suitable for a selected formulation and intended mode of administration and treatment.

For example, the container(s) include the composition of the disclosure, and optionally in addition with therapeutic regimens or agents disclosed herein. Such kits optionally include an identifying description or label or instructions relating to its use in the methods described herein.

A kit typically includes labels listing contents and/or instructions for use, and package inserts with instructions for use. A set of instructions will also typically be included.

In embodiments, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### EXAMPLES

The following examples are included for illustrative purposes only and are not intended to limit the scope of the disclosure.

### Example 1: Zinc Finger Design

Zinc finger binding sites were selected based on the availability of zinc finger modules, their location and orientation in the target gene of interest. For example, in a sequence comprising the EF1alpha promoter driving expression of GFP, an exemplary sequence contains the 3′ 200 base pairs of the EF1alpha promoter, the 23 base pairs between the promoter and the GFP start codon and the 5′ 177 base pairs of the GFP coding sequence. Exemplary binding sites for 6-finger zinc finger proteins are in “Target Site Table” and are shown in bold, or in italics when the binding site overlaps with another binding site in SEQ ID NO.: 695, shown below:

GTACGTCGTCTTTAGGTTGGGGGGAGGGGTTTTATGCGATGGAGTTTCC
CCACACTGAGTGGGTGGAGACTGAAGTTAGGCCAGCTTGGCACTTGATG
TAATTCTCCTTGGAATTTGCCCTTTTTGAGTTTGGATCTTGGTTCATTC
TCAAGCCTCAGACAGTGGTTCAAAGTTTTTTTCTTCCATTTCAGGTGTC
GTGACGCTAGCGCTACCGGTCGCCACCATGGTGAGCAAGGGCGCCGAGC
TGTTCACCGGCATCGTGCCCATCCTGATCGAGCTGAATGGCGATGTGAA
TGGCCACAAGTTCAGCGTGAGCGGCGAGGGCGAGGGCGATGCCACCTAC
GGCAAGCTGACCCTGAAGTTCATCTGCACCACCGGCAAGCTGCCTGTGC
CCTGGCCC

TABLE 7
Target Site Table

| Target Site | Sequence |
|---|---|
| GFP-1 | SEQ ID NO.: 696 |
| GFP-2 | SEQ ID NO.: 697 |
| GFP-3 | SEQ ID NO.: 698 |
| GFP-4 | SEQ ID NO.: 699 |
| GFP-5 | SEQ ID NO.: 700 |
| GFP-6 | SEQ ID NO.: 701 |
| GFP-7 | SEQ ID NO.: 702 |

Zinc finger sequences were designed for binding of the above described target site. Exemplary Zinc finger sequences are as follows: SRPGERPFQCRICMRNFS[F1]HTRTHTGEKPFQCRICMRNFS[F2]HLRTH[linker1]FQCRIC MRNFS[F3]HTRTHTGEKPFQCRICMRNFS[F4]HLRTH[linker2]FQCRICMRNFS[F5]HTRT HTGEKPFQCRICMRNFS[F6]HLRTHLRGS (SEQ ID NO.: 703)
Where zinc finger proteins for a given target site have the following linkers:

TABLE 8
Linkers for a Given Target Site

| Target Site | Sequence | Linker 1 | Linker 2 |
|---|---|---|---|
| GFP-1 | SEQ ID NO.: 696 | SEQ ID NO.: 704 | SEQ ID NO.: 705 |
| GFP-2 | SEQ ID NO.: 697 | SEQ ID NO.: 705 | SEQ ID NO.: 704 |
| GFP-3 | SEQ ID NO.: 698 | SEQ ID NO.: 704 | SEQ ID NO.: 705 |
| GFP-4 | SEQ ID NO.: 699 | SEQ ID NO.: 704 | SEQ ID NO.: 704 |
| GFP-5 | SEQ ID NO.: 700 | SEQ ID NO.: 704 | SEQ ID NO.: 704 |
| GFP-6 | SEQ ID NO.: 701 | SEQ ID NO.: 704 | SEQ ID NO.: 704 |
| GFP-7 | SEQ ID NO.: 702 | SEQ ID NO.: 704 | SEQ ID NO.: 705 |

and where recognition helices for a given target site may be selected from the following SEQ ID NO.: 716-961:

TABLE 9
Recognition Helices for a Given Target Site

| Target | Zinc Finger | | | | | | |
|---|---|---|---|---|---|---|---|
| Site | Protein Name | F1 | F2 | F3 | F4 | F5 | F6 |
| GFP-1 | GFP1-ZF1 | HKSSLTR | RTEHLAR | QSAHLKR | RTEHLAR | HKSSLTR | RPESLAP |
| | | (SEQ ID | (SEQ ID | (SEQ ID | (SEQ ID | (SEQ ID | (SEQ ID NO: |
| | | NO: 716) | NO: 757) | NO: 798) | NO: 839) | NO: 880) | 921) |
| | GFP1-ZF2 | HKSSLTR | RTEHLAR | TSAHLAR | RREHLVR | HKSSLTR | RPESLAP |
| | | (SEQ ID | (SEQ ID | (SEQ ID | (SEQ ID | (SEQ ID | (SEQ ID NO: |
| | | NO: 717) | NO: 758) | NO: 799) | NO: 840) | NO: 881) | 922) |
| | GFP1-ZF3 | IKAILTR | RREHLVR | QSAHLKR | RTEHLAR | HKSSLTR | RPESLAP |
| | | (SEQ ID | (SEQ ID | (SEQ ID | (SEQ ID | (SEQ ID | (SEQ ID NO: |
| | | NO: 718) | NO: 759) | NO: 800) | NO: 841) | NO: 882) | 923) |
| | GFP1-ZF4 | IKAILTR | RREHLVR | TSAHLAR | RREHLVR | HKSSLTR | RPESLAP |
| | | (SEQ ID | (SEQ ID | (SEQ ID | (SEQ ID | (SEQ ID | (SEQ ID NO: |
| | | NO: 719) | NO: 760) | NO: 801) | NO: 842) | NO: 883) | 924) |
| GFP-2 | GFP2-ZF1 | TSTLLNR | QQTNLTR | DEANLRR | QSAHLKR | IPNKLAR | RREVLEN |
| | | (SEQ ID | (SEQ ID | (SEQ ID | (SEQ ID | (SEQ ID | (SEQ ID NO: |
| | | NO: 720) | NO: 761) | NO: 802) | NO: 843) | NO: 884) | 925) |
| | GFP2-ZF2 | TSTLLNR | QQTNLTR | DEANLRR | QSAHLKR | EAHHLSR | RKDALHV |
| | | (SEQ ID | (SEQ ID | (SEQ ID | (SEQ ID | (SEQ ID | (SEQ ID NO: |
| | | NO: 721) | NO: 762) | NO: 803) | NO: 844) | NO: 885) | 926) |
| | GFP2-ZF3 | TSTLLNR | QQTNLTR | DRGNLTR | QGGHLKR | IPNKLAR | RREVLEN |
| | | (SEQ ID | (SEQ ID | (SEQ ID | (SEQ ID | (SEQ ID | (SEQ ID NO: |
| | | NO: 722) | NO: 763) | NO: 804) | NO: 845) | NO: 886) | 927) |
| | GFP2-ZF4 | TSTLLNR | QQTNLTR | DRGNLTR | QGGHLKR | EAHHLSR | RKDALHV |
| | | (SEQ ID | (SEQ ID | (SEQ ID | (SEQ ID | (SEQ ID | (SEQ ID NO: |
| | | NO: 723) | NO: 764) | NO: 805) | NO: 846) | NO: 887) | 928) |
| | GFP2-ZF5 | HKSSLTR | QTNNLGR | DEANLRR | QSAHLKR | IPNKLAR | RREVLEN |
| | | (SEQ ID | (SEQ ID | (SEQ ID | (SEQ ID | (SEQ ID | (SEQ ID NO: |
| | | NO: 724) | NO: 765) | NO: 806) | NO: 847) | NO: 888) | 929) |
| | GFP2-ZF6 | HKSSLTR | QTNNLGR | DEANLRR | QSAHLKR | EAHHLSR | RKDALHV |
| | | (SEQ ID | (SEQ ID | (SEQ ID | (SEQ ID | (SEQ ID | (SEQ ID NO: |
| | | NO: 725) | NO: 766) | NO: 807) | NO: 848) | NO: 889) | 930) |
| | GFP2-ZF7 | HKSSLTR | QTNNLGR | DRGNLTR | QGGHLKR | IPNKLAR | RREVLEN |
| | | (SEQ ID | (SEQ ID | (SEQ ID | (SEQ ID | (SEQ ID | (SEQ ID NO: |
| | | NO: 726) | NO: 767) | NO: 808) | NO: 849) | NO: 890) | 931) |
| | GFP2-ZF8 | HKSSLTR | QTNNLGR | DRGNLTR | QGGHLKR | EAHHLSR | RKDALHV |
| | | (SEQ ID | (SEQ ID | (SEQ ID | (SEQ ID | (SEQ ID | (SEQ ID NO: |
| | | NO: 727) | NO: 768) | NO: 809) | NO: 850) | NO: 891) | 932) |
| GFP-3 | GFP3-ZF1 | QQTNLTR | IRHHLKR | DSSVLRR | LSTNLTR | QSTTLKR | RSDHLSL |
| | | (SEQ ID | (SEQ ID | (SEQ ID | (SEQ ID | (SEQ ID | (SEQ ID NO: |
| | | NO: 728) | NO: 769) | NO: 810) | NO: 851) | NO: 892) | 933) |
| | GFP3-ZF2 | QQTNLTR | IRHHLKR | DGSTLNR | VRHNLTR | QSTTLKR | RSDHLSL |
| | | (SEQ ID | (SEQ ID | (SEQ ID | (SEQ ID | (SEQ ID | (SEQ ID NO: |
| | | NO: 729) | NO: 770) | NO: 811) | NO: 852) | NO: 893) | 934) |
| | GFP3-ZF3 | RKPNLLR | EAHHLSR | DSSVLRR | LSTNLTR | QSTTLKR | RSDHLSL |
| | | (SEQ ID | (SEQ ID | (SEQ ID | (SEQ ID | (SEQ ID | (SEQ ID NO: |
| | | NO: 730) | NO: 771) | NO: 812) | NO: 853) | NO: 894) | 935) |
| | GFP3-ZF4 | RKPNLLR | EAHHLSR | DGSTLNR | VRHNLTR | QSTTLKR | RSDHLSL |
| | | (SEQ ID | (SEQ ID | (SEQ ID | (SEQ ID | (SEQ ID | (SEQ ID NO: |
| | | NO: 731) | NO: 772) | NO: 813) | NO: 854) | NO: 895) | 936) |
| GFP-4 | GFP4-ZF1 | VRHNLTR | ESGHLKR | RQDNLGR | KNHSLNN | RQDNLGR | KNHSLNN |
| | | (SEQ ID | (SEQ ID | (SEQ ID | (SEQ ID | (SEQ ID | (SEQ ID NO: |
| | | NO: 732) | NO: 773) | NO: 814) | NO: 855) | NO: 896) | 937) |
| GFP-5 | GFP5-ZF1 | DSSVLRR | LSTNLTR | LKEHLTR | RVDNLPR | LKEHLTR | RVDNLPR |
| | | (SEQ ID | (SEQ ID | (SEQ ID | (SEQ ID | (SEQ ID | (SEQ ID NO: |
| | | NO: 733) | NO: 774) | NO: 815) | NO: 856) | NO: 897) | 938) |
| | GFP5-ZF2 | DSSVLRR | LSTNLTR | LKEHLTR | RVDNLPR | SPSKLVR | RQDNLGR |
| | | (SEQ ID | (SEQ ID | (SEQ ID | (SEQ ID | (SEQ ID | (SEQ ID NO: |
| | | NO: 734) | NO: 775) | NO: 816) | NO: 857) | NO: 898) | 939 |
| | GFP5-ZF3 | DSSVLRR | LSTNLTR | SPSKLVR | RQDNLGR | LKEHLTR | RVDNLPR |
| | | (SEQ ID | (SEQ ID | (SEQ ID | (SEQ ID | (SEQ ID | (SEQ ID NO: |
| | | NO: 735) | NO: 776) | NO: 817) | NO: 858) | NO: 899) | 940) |
| | GFP5-ZF4 | DSSVLRR | LSTNLTR | SPSKLVR | RQDNLGR | SPSKLVR | RQDNLGR |
| | | (SEQ ID | (SEQ ID | (SEQ ID | (SEQ ID | (SEQ ID | (SEQ ID NO: |
| | | NO: 736) | NO: 777) | NO: 818) | NO: 859) | NO: 900) | 941) |
| | GFP5-ZF5 | DGSTLNR | VRHNLTR | LKEHLTR | RVDNLPR | LKEHLTR | RVDNLPR |
| | | (SEQ ID | (SEQ ID | (SEQ ID | (SEQ ID | (SEQ ID | (SEQ ID NO: |
| | | NO: 737) | NO: 778) | NO: 819) | NO: 860) | NO: 901) | 942) |
| | GFP5-ZF6 | DGSTLNR | VRHNLTR | LKEHLTR | RVDNLPR | SPSKLVR | RQDNLGR |
| | | (SEQ ID | (SEQ ID | (SEQ ID | (SEQ ID | (SEQ ID | (SEQ ID NO: |
| | | NO: 738) | NO: 779) | NO: 820) | NO: 861) | NO: 902) | 943) |
| | GFP5-ZF7 | DGSTLNR | VRHNL TR | SPSKLVR | RQDNLGR | LKEHLTR | RVDNLPR |
| | | (SEQ ID | (SEQ ID | (SEQ ID | (SEQ ID | (SEQ ID | (SEQ ID NO: |
| | | NO: 739) | NO: 780) | NO: 821) | NO: 862) | NO: 903) | 944) |
| | GFP5-ZF8 | DGSTLNR | VRHNLTR | SPSKLVR | RQDNLGR | SPSKLVR | RQDNLGR |
| | | (SEQ ID | (SEQ ID | (SEQ ID | (SEQ ID | (SEQ ID | (SEQ ID NO: |
| | | NO: 740) | NO: 781) | NO: 822) | NO: 863) | NO: 904) | 945) |
| GFP-6 | GFP6-ZF1 | RKPNLLR | VRHNLTR | DKAQLGR | EAHHLSR | RQSRLQR | KGDHLRR |
| | | (SEQ ID | (SEQ ID | (SEQ ID | (SEQ ID | (SEQ ID | (SEQ ID NO: |
| | | NO: 741) | NO: 782) | NO: 823) | NO: 864) | NO: 905) | 946) |
| | GFP6-ZF2 | RKPNLLR | VRHNLTR | DKAQLGR | EAHHLSR | EAHHLSR | DPSNLRR |
| | | (SEQ ID | (SEQ ID | (SEQ ID | (SEQ ID | (SEQ ID | (SEQ ID NO: |
| | | NO: 742) | NO: 783) | NO: 824) | NO: 865) | NO: 906) | 947) |
| | GFP6-ZF3 | RKPNLLR | VRHNLTR | QSTTLKR | VDHHLRR | RQSRLQR | KGDHLRR |
| | | (SEQ ID | (SEQ ID | (SEQ ID | (SEQ ID | (SEQ ID | (SEQ ID NO: |
| | | NO: 743) | NO: 784) | NO: 825) | NO: 866) | NO: 907) | 948) |
| | GFP6-ZF4 | RKPNLLR | VRHNLTR | QSTTLKR | VDHHLRR | EAHHLSR | DPSNLRR |
| | | (SEQ ID | (SEQ ID | (SEQ ID | (SEQ ID | (SEQ ID | (SEQ ID NO: |
| | | NO: 744) | NO: 785) | NO: 826) | NO: 867) | NO: 908) | 949) |
| | GFP6-ZF5 | QQTNLTR | VGSNLTR | DKAQLGR | EAHHLSR | RQSRLQR | KGDHLRR |
| | | (SEQ ID | (SEQ ID | (SEQ ID | (SEQ ID | (SEQ ID | (SEQ ID NO: |
| | | NO: 745) | NO: 786) | NO: 827) | NO: 868) | NO: 909) | 950) |
| | GFP6-ZF6 | QQTNLTR | VGSNLTR | DKAQLGR | EAHHLSR | EAHHLSR | DPSNLRR |
| | | (SEQ ID | (SEQ ID | (SEQ ID | (SEQ ID | (SEQ ID | (SEQ ID NO: |
| | | NO: 746) | NO: 787) | NO: 828) | NO: 869) | NO: 910) | 951) |
| | GFP6-ZF7 | QQTNLTR | VGSNLTR | QSTTLKR | VDHHLRR | RQSRLQR | KGDHLRR |
| | | (SEQ ID | (SEQ ID | (SEQ ID | (SEQ ID | (SEQ ID | (SEQ ID NO: |
| | | NO: 747) | NO: 788) | NO: 829) | NO: 870) | NO: 911) | 952) |
| | GFP6-ZF8 | QQTNLTR | VGSNLTR | QSTTLKR | VDHHLRR | EAHHLSR | DPSNLRR |
| | | (SEQ ID | (SEQ ID | (SEQ ID | (SEQ ID | (SEQ ID | (SEQ ID NO: |
| | | NO: 748) | NO: 789) | NO: 830) | NO: 871) | NO: 912) | 953) |
| GFP-7 | GFP7-ZF1 | QSTTLKR | VDHHLRR | EAHHLSR | DPSNLRR | QRSDLTR | QGGTLRR |
| | | (SEQ ID | (SEQ ID | (SEQ ID | (SEQ ID | (SEQ ID | (SEQ ID NO: |
| | | NO: 749) | NO: 790) | NO: 831) | NO: 872) | NO: 913) | 954) |
| | GFP7-ZF2 | QSTTLKR | VDHHLRR | EAHHLSR | DPSNLRR | TKQILGR | QSTTLKR |
| | | (SEQ ID | (SEQ ID | (SEQ ID | (SEQ ID | (SEQ ID | (SEQ ID NO: |
| | | NO: 750) | NO: 791) | NO: 832) | NO: 873) | NO: 914) | 955) |
| | GFP7-ZF3 | QSTTLKR | VDHHLRR | RQSRLQR | DSSVLRR | QRSDLTR | QGGTLRR |
| | | (SEQ ID | (SEQ ID | (SEQ ID | (SEQ ID | (SEQ ID | (SEQ ID NO: |
| | | NO: 751) | NO: 792) | NO: 833) | NO: 874) | NO: 915) | 956) |
| | GFP7-ZF4 | QSTTLKR | VDHHLRR | RQSRLQR | DSSVLRR | TKQILGR | QSTTLKR |
| | | (SEQ ID | (SEQ ID | (SEQ ID | (SEQ ID | (SEQ ID | (SEQ ID NO: |
| | | NO: 752) | NO: 793) | NO: 834) | NO: 875) | NO: 916) | 957) |
| | GFP7-ZF5 | DKAQLGR | EAHHLSR | EAHHLSR | DPSNLRR | QRSDLTR | QGGTLRR |
| | | (SEQ ID | (SEQ ID | (SEQ ID | (SEQ ID | (SEQ ID | (SEQ ID NO: |
| | | NO: 753) | NO: 794) | NO: 835) | NO: 876) | NO: 917) | 958) |
| | GFP7-ZF6 | DKAQLGR | EAHHL SR | EAHHLSR | DPSNLRR | TKQILGR | QSTTLKR |
| | | (SEQ ID | (SEQ ID | (SEQ ID | (SEQ ID | (SEQ ID | (SEQ ID NO: |
| | | NO: 754) | NO: 795) | NO: 836) | NO: 877) | NO: 918) | 959) |
| | GFP7-ZF7 | DKAQLGR | EAHHLSR | RQSRLQR | DSSVLRR | QRSDLTR | QGGTLRR |
| | | (SEQ ID | (SEQ ID | (SEQ ID | (SEQ ID | (SEQ ID | (SEQ ID NO: |
| | | NO: 755) | NO: 796) | NO: 837) | NO: 878) | NO: 919) | 960) |
| | GFP7-ZF8 | DKAQLGR | EAHHLSR | RQSRLQR | DSSVLRR | TKQILGR | QSTTLKR |
| | | (SEQ ID | (SEQ ID | (SEQ ID | (SEQ ID | (SEQ ID | (SEQ ID NO: |
| | | NO: 756) | NO: 797) | NO: 838) | NO: 879) | NO: 920) | 961) |

### Example 2: Epigenetic Editor Sequences

Amino acid sequences of exemplary epigenetic editors are provided below. Exemplary fusion protein DNMT3A-3L-ZF-KRAB (SEQ ID NO.: 978) where zinc finger is GFP1-ZF1:

MAPKKKRKMNHDQEFDPPKVYPPVPAEKRKPIRVLSLEDGIATGLLVLK
DLGIQVDRYIASEVCEDSITVGMVRHQGKIMYVGDVRSVTQKHIQEWGP
FDLVIGGSPCNDLSIVNPARKGLYEGTGRLFFEFYRLLHDARPKEGDDR
PFFWLFENVVAMGVSDKRDISRFLESNPVMIDAKEVSAAHRARYFWGNL
PGMNRPLASTVNDKLELQECLEHGRIAKFSKVRTITTRSNSIKQGKDQH
FPVFMNEKEDILWCTEMERVFGFPVHYTDVSNMSRLARQRLLGRSWSVP
VIRHLFAPLKEYFACVSSGNSNANSRGPSFSSGLVPLSLRGSHMGPMEI
YKTVSAWKRQPVRVLSLFRNIDKVLKSLGFLESGSGSGGGTLKYVEDVT
NVVRRDVEKWGPFDLVYGSTQPLGSSCDRCPGWYMFQFHRILQYALPRQ
ESQRPFFWIFMDNLLLTEDDQETTTRFLQTEAVTLQDVRGRDYQNAMRV
WSNIPGLKSKHAPLTPKEEEYLQAQVRSRSKLDAPKVDLLVKNCLLPLR
EYFKYFSQNSLPLSGGGGSGGGGSVGIHGVPSRPGERPFQCRICMRNFS
HKSSLTRHTRTHTGEKPFQCRICMRNFSRTEHLARHLRTHTGSQKPFQC
RICMRNFSQSAHLKRHTRTHTGEKPFQCRICMRNFSRTEHLARHLRTHT
GGGGSQKPFQCRICMRNFSHKSSLTRHTRTHTGEKPFQCRICMRNFSRP
ESLAPHLRTHLRGSGGGSMDAKSLTAWSRTLVTFKDVFVDFTREEWKLL
DTAQQIVYRNVMLENYKNLVSLGYQLTKPDVILRLEKGEEPWLVEREIH
QETHPDSETAFEIKSSV
(italics: DNMT3A; Bold: DNMT3L; underline:KRAB)

### Example 3: Guide RNA Design

Cas9 protospacers are chosen based on homology from sequences that perfectly match or nearly perfectly match spacer sequences in target DNA sequences and predicted by the MIT Specificity Score (calculated by http://crispor.tefor.net/).

gRNA protospacer sequences that would permit epigenetic editors containing a Streptococcus pyogenes Cas9, or another Cas that can use the NGG PAM, to recognize the protospacer sequences identified throughout the target gene. gRNAs containing spacers of 20 nts and a total length of 100 nts are synthesized. gRNAs are co-transfected with mRNA encoding the Cas9 epigenetic editor fusion protein into primary human hepatocytes via MessengerMax reagent (Lipofectamine). After transfection, genomic DNA from the hepatocytes is harvested, and transcript expression level of the target gene was assessed by qRT-PCR.

### Example 4: Epigenetic Editor Mediated Repression of Target Gene

Candidate zinc fingers are screened as described using ZiFit (http://bindr.gdcb.iastate.edu/ZiFiT/). Human K562 cells are cultured in RPMI 1640 medium (Gibco) with 10% HI-FBS (Gibco), 1% Glutamax (Gibco) and 1% Pen/Strep (Gibco), and are transfected with plasmids encoding various KRAB-ZF-Dnmt3A-Dnmt3L fusion proteins by nucleofecting 1×10\hat{ }6 dividing cells with 10 μg of DNA in 100 μl of Kit V solution (Lonza) using program T-016 on the Nucleofector 2b Device (Lonza). Nucleofected cells are incubated in 6-well plates at 370 C for 4 days following nucleofection. Genomic DNA and total RNA are harvested 4 days post-transfection. Genomic DNA is used for methylation analysis. Total RNA is extracted and the expression of the target gene and two reference genes (ATP5b and RPL38) are monitored using real-time RT-qPCR.

Methylation state determination: Bisulfite DNA sequencing of the target gene locus from these transfected cell populations are performed as follows. Genomic DNA is isolated from transfected cells using the Qiagen Blood Mini kit. 200-1000 ng of genomic DNA is bisulfite treated using either the EZ DNA Methylation Kit (Zymo), EZ DNA Methylation-Lightning Kit (Zymo), or Cells-to-CpG Bisulfite Conversion Kit (Applied Biosystems) following recommended protocols. PCR amplification of Bis-DNA is performed using Pyromark PCR kit (Qiagen). Illumina adapters and barcodes are added by PCR with Phusion High-Fidelity PCR enzyme (NEB) and amplicons were sequenced on an Illumina MiSeq system. Total RNA is isolated from the same cells with the PureLink RNA mini kit (Ambion) according to manufacturer's instructions. Reverse transcription is performed with the Superscriptlll RT kit (Invitrogen) and Tagman assays were run on an Applied Biosystems 7500Fast Real Time PCR machine.

Testing Repression Domains: To test the functionality of candidate repression domains, the domain is fused to a DNA-binding domain for testing in human cells. The effector domain, identified and extracted from the full protein sequence may be fused to the N-terminal or C-terminal end of any DNA-binding domain, using a variety of linkers. For example, a repressor domain may be fused to Cas9. This fusion protein is then co-delivered into cells, along with a gRNA, using standard cell culture techniques. This may include plasmid transfection or electroporation, mRNA transfection or electroporation, or viral transduction. Initial testing of effector domains can easily be performed in reporter cell lines in which a fluorescent marker has been integrated to enable easy FACS-based readout. Alternatively, endogenous genes can be targeted. Genes encoding cell surface markers can be easily quantified by flow cytometry and expression of any gene target can be quantified by standard molecular biology techniques such as RT-qPCR, ddPCR, Western blot, etc. To test candidate repression domains, decreased expression of the target gene is quantified by these methods. Truncations and mutations can be introduced into the effector domain to generate multiple variants for testing.

Testing Activation Domains: To test the functionality of candidate activation domains, the domain is fused to a DNA-binding domain for testing in human cells. The effector domain, identified and extracted from the full protein sequence may be fused to the N-terminal or C-terminal end of any DNA-binding domain, using a variety of linkers. For example, an activation domain may be fused to Cas9. This fusion protein is then co-delivered into cells, along with a gRNA, using standard cell culture techniques. This may include plasmid transfection or electroporation, mRNA transfection or electroporation, or viral transduction. Initial testing of effector domains can easily be performed in reporter cell lines in which a fluorescent marker has been integrated to enable easy FACS-based readout. Alternatively, endogenous genes can be targeted. Genes encoding cell surface markers can be easily quantified by flow cytometry and expression of any gene target can be quantified by standard molecular biology techniques such as RT-qPCR, ddPCR, Western blot, etc. To test candidate activation domains, increased expression of the target gene is quantified by these methods. Truncations and mutations can be introduced into the effector domain to generate multiple variants for testing.

Testing DNA methyltransferase domains: To test the functionality of candidate DNA methyltransferase domains, the domain is fused to a DNA-binding domain for testing in human cells. The effector domain, identified and extracted from the full protein sequence may be fused to the N-terminal or C-terminal end of any DNA-binding domain, using a variety of linkers. For example, a DNA methyltransferase domain may be fused to Cas9. This fusion protein is then co-delivered into cells, along with a gRNA, using standard cell culture techniques. This may include plasmid transfection or electroporation, mRNA transfection or electroporation, or viral transduction. Because DNA methylation is expected to reduce target gene expression, this may be assayed by standard techniques such as RT-qPCR, staining for cell surface marker and quantifying by flow cytometry, ddPCR and Western blotting. Additionally, direct readout of DNA methylation is obtained through bisulfite sequencing. In this method, bisulfite treatment of DNA converts cytosine residues to uracil but leaves 5-methylcytosine residues unaffected. Standard Sanger sequencing or next-generation sequencing can then be performed to determine the rate of methylation at CpG dinucleotides.

Testing DNA demethylation domains: To test the functionality of candidate domains for removing DNA methylation, the domain is fused to a DNA-binding domain for testing in human cells. The effector domain, identified and extracted from the full protein sequence may be fused to the N-terminal or C-terminal end of any DNA-binding domain, using a variety of linkers. For example, a domain may be fused to Cas9. This fusion protein is then co-delivered into cells, along with a gRNA, using standard cell culture techniques. This may include plasmid transfection or electroporation, mRNA transfection or electroporation, or viral transduction. Because removal of DNA methylation marks at CpG dinucleotides is expected to increase target gene expression, this may be assayed by standard techniques such as RT-qPCR, staining for cell surface marker and quantifying by flow cytometry, ddPCR and Western blotting. Additionally, direct readout of DNA methylation is obtained through bisulfite sequencing. In this method, bisulfite treatment of DNA converts cytosine residues to uracil but leaves 5-methylcytosine residues unaffected. Standard Sanger sequencing or next-generation sequencing can then be performed to determine the rate of methylation at CpG dinucleotides.

### Example 5: Alternate DNMT Effectors and Effector Fusions

GripTite293 cells were seeded in 96-well plates and transfected with 25 ng of a gRNA-expressing plasmid (targeting VIM), 50 ng of an Effector-DBD fusion plasmid, and 5 ng of a Puromycin resistance plasmid using Mirus TransIT transfection reagent. VIM-targeting gRNAs used can be found in SEQ ID NO.: 962-969. Effector-DBD fusions can be found in SEQ ID NO.: 1092-1133.

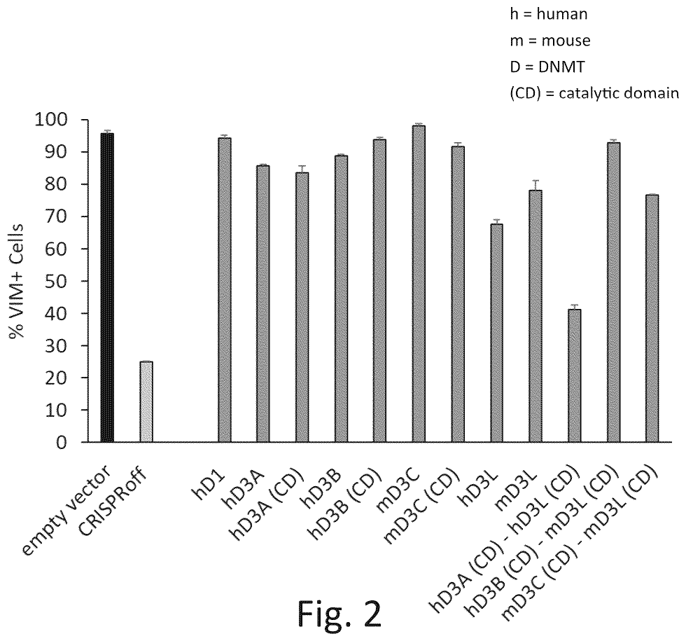

At day 1 post transfection, cells were cultured with Puromycin to select for positively transfected cells. At day 6 or day 7 post transfection, cells were analyzed for VIM expression via FACS (FIG. 2).

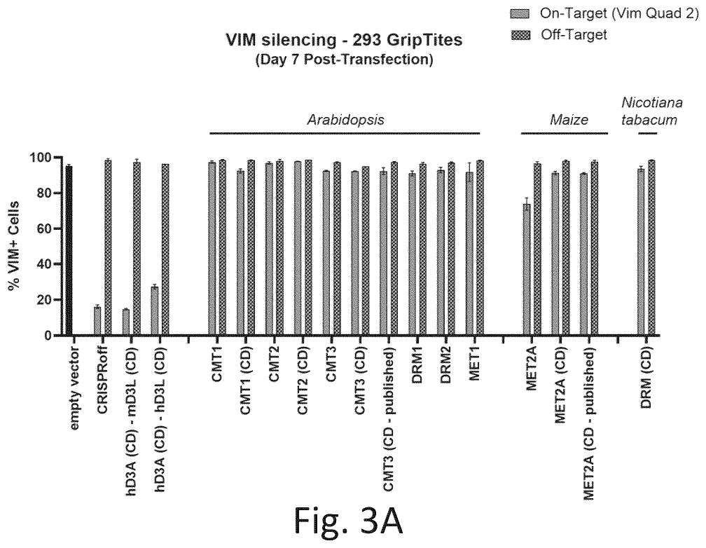

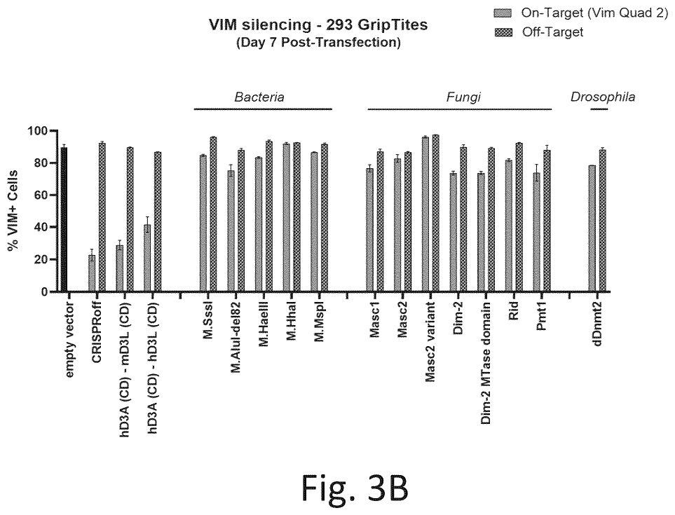

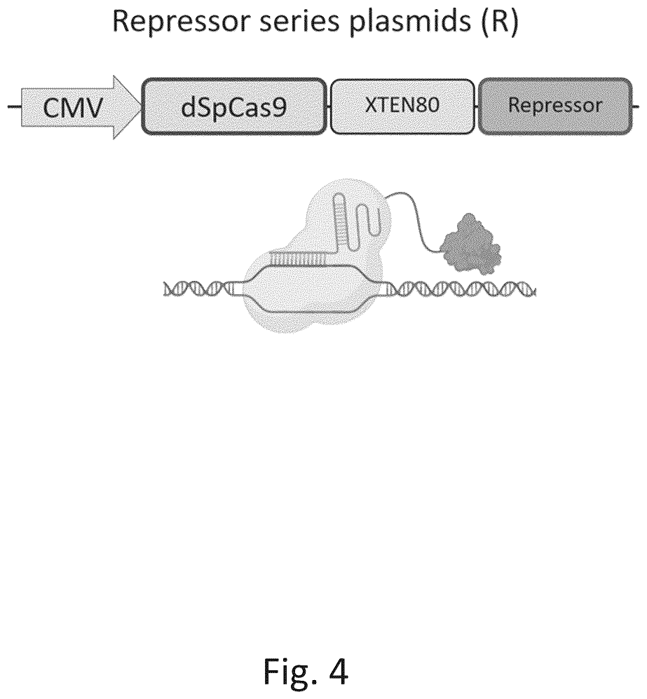

When human-human and human-mouse fusions were tested against plant DNMT effectors and effector fusions, the mammalian fusions exhibited greateer VIM silencing (FIG. 3A); similar results were found when the mammalian fusions were compared to DNMT effectors and effector fusions from bacteria, fungi, and Drosophila (FIG. 3B).

### Example 6: Alternate KRAB and Non-KRAB Repressors

GripTite293 cells were seeded in 96-well plates and transfected with 25 ng of a gRNA-expressing plasmid (targeting VIM), 50 ng of a DBD-Effector fusion plasmid, and 5 ng of a Puromycin resistance plasmid using Mirus TransIT transfection reagent. VIM-targeting gRNAs used can be found in SEQ ID NO.: 962-969. DBD-Effector fusions can be found in SEQ ID NO.: 1002-1091.

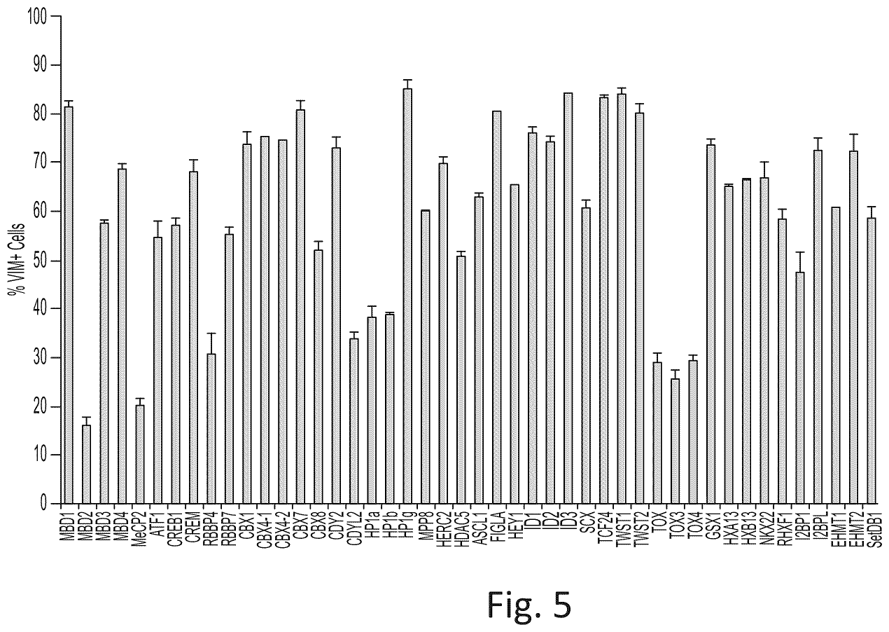

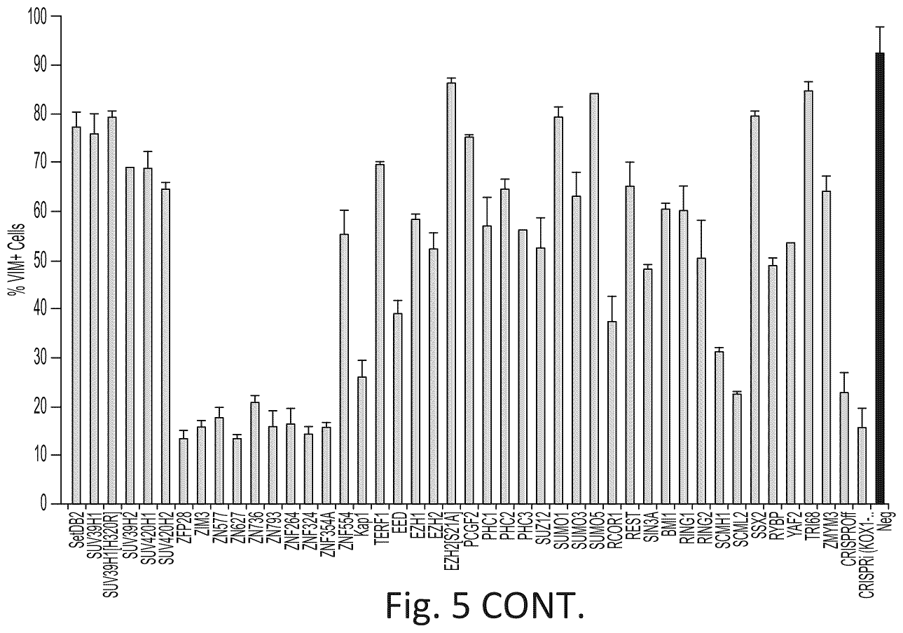

At day 1 post transfection, cells were cultured with Puromycin to select for positively transfected cells. At day 6 post transfection, cells were analyzed for VIM expression via FACS (FIG. 5). Many alternate KRAB and non-KRAB repressors effectively silenced VIM expression.

### Example 7: Gene Repression

GripTite293 cells were seeded in 96-well plates and transfected with 25 ng of a gRNA-expressing plasmid (either single gRNA or 4× (quad) gRNA plasmid targeting CD151 or CLTA), 50 ng of a DBD-Effector fusion plasmid, and 5 ng of a Puromycin resistance plasmid using Mirus TransIT transfection reagent. CD151-targeting gRNAs used can be found in SEQ ID NO.: 970-977. DBD-Effector fusion plasmids used can be found in SEQ ID NO.: 978-1001.

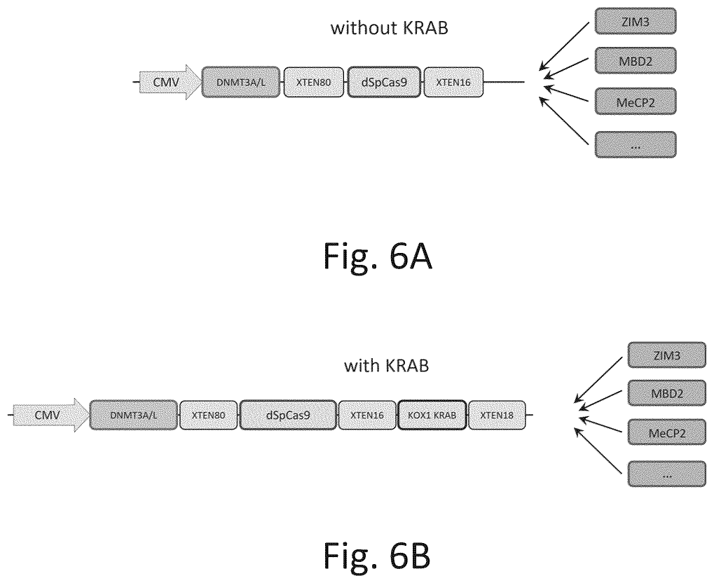

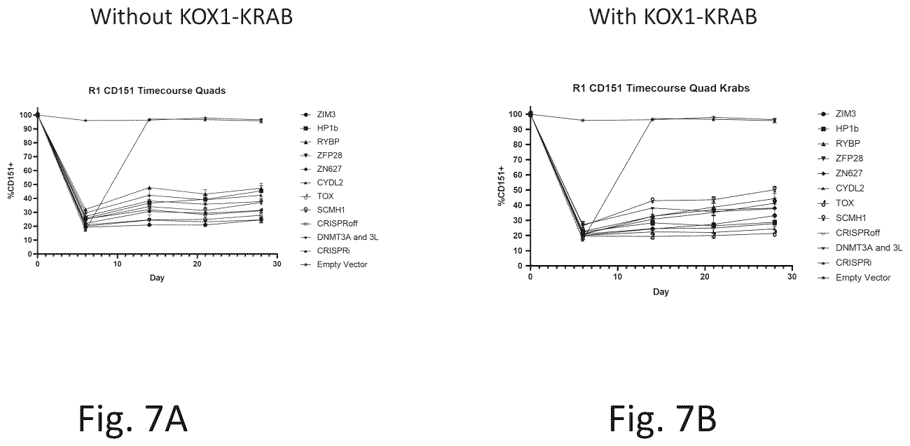

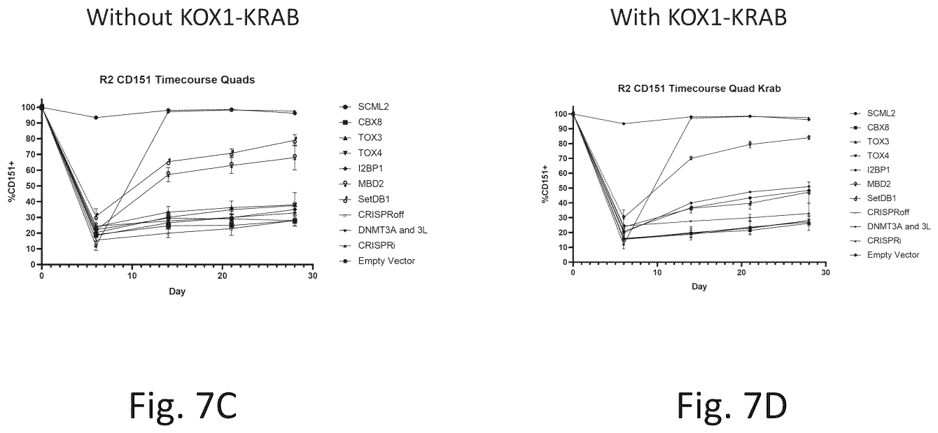

At day 1 post transfection, cells were cultured with Puromycin to select for positively transfected cells. At day 6 post transfection, cells were analyzed for CD151 or CLTA expression via FACS. FIG. 6-7 show that many of the alternate KRAB combination effectively silence CD151.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Other Embodiments

From the foregoing description, it will be apparent that variations and modifications may be made to the disclosure described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment, any portion of the embodiment, or in combination with any other embodiments or any portion thereof.

As is set forth herein, it will be appreciated that the disclosure comprises specific embodiments and examples of base editing systems to effect a nucleobase alteration in a gene and methods of using same for treatment of disease including compositions that comprise such base editing systems, designs and modifications thereto; and specific examples and embodiments describing the synthesis, manufacture, use, and efficacy of the foregoing individually and in combination including as pharmaceutical compositions for treating disease and for in vivo and in vitro delivery of active agents to mammalian cells under described conditions.

While specific examples and numerous embodiments have been provided to illustrate aspects and combinations of aspects of the foregoing, it should be appreciated and understood that any aspect, or combination thereof, of an exemplary or disclosed embodiment may be excluded therefrom to constitute another embodiment without limitation and that it is contemplated that any such embodiment can constitute a separate and independent claim. Similarly, it should be appreciated and understood that any aspect or combination of aspects of one or more embodiments may also be included or combined with any aspect or combination of aspects of one or more embodiments and that it is contemplated herein that all such combinations thereof fall within the scope of this disclosure and can be presented as separate and independent claims without limitation. Accordingly, it should be appreciated that any feature presented in one claim may be included in another claim; any feature presented in one claim may be removed from the claim to constitute a claim without that feature; and any feature presented in one claim may be combined with any feature in another claim, each of which is contemplated herein. The following enumerated clauses are further illustrative examples of aspects and combination of aspects of the foregoing embodiments and examples:
1. A method of modifying an epigenetic state of a target gene in a target chromosome, the method comprising contacting the target chromosome with an epigenetic editor, wherein the epigenetic editor comprises a DNA binding domain and an epigenetic effector domain, wherein the DNA binding domain binds to a target sequence in the target chromosome and directs the epigenetic effector domain to effect a site-specific epigenetic modification in the target gene or a histone bound to the target gene in the target chromosome, thereby modifying the epigenetic state of the target gene.
2. A method of modulating expression of a target gene in a target chromosome in a cell, the method comprising contacting the target gene with an epigenetic editor, wherein the epigenetic editor comprises a DNA binding domain and an epigenetic effector domain, wherein the DNA binding domain binds to a target sequence in the target chromosome and directs the epigenetic effector domain to effect a site-specific epigenetic modification in the target gene or a histone bound to the target gene, thereby modulating expression of the target gene.
3. A method of treating a disease in a subject in need thereof, the method comprising administering to the subject an epigenetic editor, wherein the epigenetic editor comprises a DNA binding domain and an epigenetic effector domain, wherein the DNA binding domain binds to a target sequence in a target chromosome comprising a target gene in the subject and directs the epigenetic effector domain to effect a site-specific epigenetic modification in the target gene or a histone bound to the target gene, wherein the target gene is associated with disease and wherein the site-specific epigenetic modification modulates expression of the target gene, thereby treating the disease.
4. The method of any one of the preceding claims, wherein the site-specific epigenetic modification is within 3000 base pairs upstream or downstream of the target sequence.
5. The method of claim 4, wherein the site-specific epigenetic modification is within 2000 base pairs upstream or downstream of the target sequence.
6. The method of any one of the preceding claims, wherein the site-specific epigenetic modification is within 3000 base pairs upstream or downstream of an expression regulatory sequence.
7. The method of claim 6, wherein the site-specific epigenetic modification is within 2000 base pairs upstream or downstream of the expression regulatory sequence.
8. The method of claim 7, wherein the site-specific epigenetic modification is within 1000 base pairs upstream or downstream of the expression regulatory sequence.
9. A method of modifying an epigenetic state of a target gene in a target chromosome, the method comprising contacting the target gene with an epigenetic editor, wherein the epigenetic editor comprises a DNA biding domain and an epigenetic effector domain, wherein the DNA biding domain binds to a target sequence in the target chromosome, and wherein the epigenetic effector domain results in an epigenetic modification in at least 10% of all nucleotides or all histone tails bound with nucleotides within 200 base pairs upstream or downstream of the target sequence in the target genome.
10. A method of modulating expression of a target gene in a target chromosome in a cell, the method comprising contacting the target gene with an epigenetic editor, wherein the epigenetic editor comprises a DNA binding domain and an epigenetic effector domain, wherein the DNA binding domain binds to a target sequence in the target chromosome, and wherein the epigenetic effector domain results in an epigenetic modification in at least 10% of all nucleotides or all histone tails bound with nucleotides within 200 base pairs upstream or downstream of the target sequence in a target genome in the cell.
11. A method of treating a disease in a subject in need thereof, the method comprising administering to the subject an epigenetic editor, wherein the epigenetic editor comprises a DNA binding domain and an epigenetic effector domain, wherein the DNA binding domain binds to a target sequence in a target chromosome comprising a target gene in the subject, wherein the epigenetic effector domain results in an epigenetic modification in at least 10% of all nucleotides or at least 10% of all histone tails bound with nucleotides within 200 base pairs upstream or downstream of the target sequence in a target genome in the subject, wherein the target gene is associated with the disease and wherein the epigenetic modification modulates expression of the target gene, thereby treating the disease.
12. The method of any one of claims 9-11, wherein the epigenetic effector domain results in the epigenetic modification in at least 20% of all nucleotides within 200 base pairs upstream or downstream of the target sequence.
13. The method of any one of claims 9-11, wherein the epigenetic effector domain results in the epigenetic modification in at least 50% of all nucleotides within 200 base pairs upstream or downstream of the target sequence.
14. The method of any one of claims 9-11, wherein the epigenetic effector domain results in the epigenetic modification in at least 10% of all nucleotides within 500 base pairs upstream or downstream of the target sequence.
15. The method of any one of claims 9-11, wherein the epigenetic effector domain results in the epigenetic modification in at least 20% of all nucleotides within 500 base pairs upstream or downstream of the target sequence.
16. A method of modifying an epigenetic state of a target gene in a target chromosome, the method comprising contacting the target gene with an epigenetic editor, wherein the epigenetic editor comprises a DNA biding domain and an epigenetic effector domain, wherein the DNA biding domain binds to a target sequence in the target chromosome, and wherein the epigenetic effector domain results in an epigenetic modification in at least 10% of all CpG dinucleotides within 200 base pairs upstream or downstream of the target sequence in the target genome.
17. A method of modulating expression of a target gene in a target chromosome in a cell, the method comprising contacting the target gene with an epigenetic editor, wherein the epigenetic editor comprises a DNA binding domain and an epigenetic effector domain, wherein the DNA binding domain binds to a target sequence in the target chromosome, and wherein the epigenetic effector domain results in an epigenetic modification in at least 10% of all CpG dinucleotides within 200 base pairs upstream or downstream of the target sequence in a target genome in the cell.
18. A method of treating a disease in a subject in need thereof, the method comprising administering to the subject an epigenetic editor, wherein the epigenetic editor comprises a DNA binding domain and an epigenetic effector domain, wherein the DNA binding domain binds to a target sequence in a target chromosome comprising a target gene in the subject, wherein the epigenetic effector domain results in an epigenetic modification in at least 10% of all CpG dinucleotides within 200 base pairs upstream or downstream of the target sequence in a target genome in the subject, wherein the target gene is associated with disease and wherein the epigenetic modification modulates expression of the target gene, thereby treating the disease.
19. The method of any one of claims 16-18, wherein the epigenetic effector domain results in the epigenetic modification in at least 20% of all CpG dinucleotides within 200 base pairs upstream or downstream of the target sequence.
20. The method of any one of claims 16-18, wherein the epigenetic effector domain results in the epigenetic modification in at least 50% of all CpG dinucleotides within 200 base pairs upstream or downstream of the target sequence.
21. The method of any one of claims 16-18, wherein the epigenetic effector domain results in the epigenetic modification in at least 10% of all CpG dinucleotides within 500 base pairs upstream or downstream of the target sequence.
22. The method of any one of claims 16-18, wherein the epigenetic effector domain results in the epigenetic modification in at least 20% of all CpG dinucleotides within 500 base pairs upstream or downstream of the target sequence.
23. The method of any one of claims 16-18, wherein the epigenetic effector domain results in the epigenetic modification in at least 80% of all CpG dinucleotides within 200 base pairs upstream or downstream of the target sequence.
24. The method of any one of claims 9-14, wherein the epigenetic effector domain results in the epigenetic modification in at least 50% of all nucleotides within 500 base pairs upstream or downstream of an expression regulatory sequence.
25. The method of any one of claims 3-8 or 11-24, comprising administering to the subject a cell comprising the epigenetic editor.
26. The method of claim 25, wherein the cell is an allogeneic cell.
27. The method of claim 25, wherein the cell is an autologous cell.
28. The method of any one of claims 6-8 or 15-27, wherein the expression regulatory sequence comprises a promoter.
29. The method of any one of claims 6-8 or 15-27, wherein the expression regulatory sequence comprises a transcription initiation start site.
30. The method of any one of claims 6-8 or 15-27, wherein the expression regulatory sequence comprises an enhancer.
31. The method of any one of the preceding claims, wherein the epigenetic modification is within a coding region of the target gene.
32. The method of any one of the preceding claims, wherein the target gene comprises an allele associated with a disease.
33. The method of any one of the preceding claims, wherein the target gene comprises two heterozygotic copies.
34. The method of claim 33, wherein the target gene is heterozygous at an allele.
35. The method of claim 33 or 34, wherein the epigenetic modification is at one of the two heterozygotic copies and not the other.
36. The method of claim 34, wherein the epigenetic modification is at the heterozygotic allele.
37. The method of any one of the preceding claims, wherein the DNA binding domain comprises a zinc finger motif.
38. The method of any one of the preceding claims, wherein the DNA binding domain comprises a zinc finger array.
39. The method of claim 38, wherein the zinc finger array comprises at least six zinc fingers.
40. The method of claim 39, wherein the zinc finger array comprises at least three subsets of zinc fingers each comprising at least two zinc fingers.
41. The method of any one of claims 1-36, wherein the DNA binding domain comprises a nucleic acid guided DNA binding domain bound to a guide polynucleotide.
42. The method of claim 41, wherein the DNA binding domain comprises CRISPR-Cas protein bound to the guide polynucleotide.
43. The method of claim 41, wherein the guide polynucleotide hybridizes with the target sequence.
44. The method of claim 41, wherein the CRISPR-Cas protein comprises a nuclease inactive Cas9 (dCas9).
45. The method of claim 41, wherein the CRISRP-Cas protein comprises a nuclease inactive Cas12a (dCas12a) or a nuclease inactive CasX (dCasX).
46. The method of any one of the preceding claims, wherein the epigenetic effector domain results in reduced or silenced expression of the target gene as compared to a control cell not contacted with the epigenetic editor.
47. The method of claim 46, wherein the epigenetic effector domain specifically reduces or silences expression from one of the heterozygotic copies of the target gene as compared to a control gene in a cell not contacted with the epigenetic editor.
48. The method of claim 46 or 47, wherein the site-specific epigenetic modification or the epigenetic modification comprises DNA methylation.
49. The method of claim 48, wherein the site-specific epigenetic modification or the epigenetic modification is in a CpG dinucleotide.
50. The method of claim 48, wherein the CpG dinucleotide is in a CpG island.
51. The method of claim 48, wherein the CpG dinucleotide is not in a CpG island.
52. The method of claim 46 or 47, wherein the site-specific epigenetic modification or the epigenetic modification comprises de-acetylation of the histone bound to the target gene.
53. The method of claim 46 or 47, wherein the site-specific epigenetic modification or the epigenetic modification comprises methylation of the histone bound to the target gene, optionally wherein the methylation of the histone is H3K9 methylation.
54. The method of claim 46 or 47, wherein the site-specific epigenetic modification comprises demethylation of the histone bound to the target gene, optionally wherein the demethylation of the histone is H3K4 demethylation.
55. The method of any one of claims 46-54, wherein the epigenetic effector domain comprises a DNA methyltransferase domain.
56. The method of claim 55, wherein the epigenetic effector domain comprises a Dnmt1 domain, a Dnmt3A domain, a Dnmt3L domain, or a Dnmt3B domain.
57. The method of claim 56, wherein the epigenetic effector domain comprises a Dnmt3A-Dnmt3L fusion protein.
58. The method of any one of claims 46-55, wherein the epigenetic effector domain comprises transcription repressor, a DNA methyltransferase, a histone methyltransferase, a histone demethylase, a histone deacetylase, or any combination thereof.
59. The method of any one of claims 46-55, wherein the epigenetic effector domain recruits a transcription repressor, a DNA methyltransferase, a histone methyltransferase, a histone demethylase, a histone deacetylase, or any combination thereof to the target gene.
60. The method of claim 58 or 59, wherein the epigenetic effector domain comprises a KRAB domain, a KAP1 domain, a MECP2 domain, a chromoshadow domain, or a HP1 domain.
61. The method of any one of claims 58-59, wherein the epigenetic effector domain comprises a protein from Table 2 or Table 3.
62. The method of any one of claims 46-61, wherein the epigenetic editor further comprises a second epigenetic effector domain that results in reduced or silenced expression of the target gene.
63. The method of claim 62, wherein the second epigenetic effector domain comprises a DNA methyltransferase domain.
64. The method of claim 62, wherein the second epigenetic effector domain comprises a transcription repressor, a DNA methyltransferase, a histone methyltransferase, a histone demethylase, a histone deacetylase, or any combination thereof.
65. The method of claim 62, wherein the second epigenetic effector domain recruits a transcription repressor, a DNA methyltransferase, a histone methyltransferase, a histone demethylase, a histone deacetylase, or any combination thereof to the target gene.
66. The method of claim 62, wherein the second epigenetic effector domain comprises a KRAB domain, a KAP1 domain, a HP1 domain, a Dnmt3A domain, a Dnmt3L domain, or any combination thereof.
67. The method of claim 62, wherein the second epigenetic effector domain comprises a protein of Table 2 or Table 3.
68. The method of any one of claims 62-67, wherein the epigenetic effector domain and the second epigenetic effector domain synergistically reduces or silences expression of the target gene.
69. The method of any one of claims 46-68, wherein the epigenetic editor comprises a DNA methyltransferase domain and a repression domain that reduces or silences expression of the target gene.
70. The method of any one of claims 46-68, wherein the epigenetic editor comprises a DNA methyltransferase domain and a repression scaffold protein domain that recruits transcription repressor proteins to the target gene.
71. The method of any one of claims 46-68, wherein the epigenetic editor comprises a DNA methyltransferase domain and a histone deacetylase domain.
72. The method of claim 71, wherein the epigenetic editor further comprises a KRAB domain, a KAP1 domain, a HP1 domain, a chromoshadow domain, or a MECP2 domain.
73. The method of any one of claim 46-72, wherein the epigenetic editor comprises from N terminus to C terminus: (i) a Dnmt3A-Dnmt3L fusion protein domain, (ii) the DNA binding domain, and (iii) a KRAB domain, a KAP1 domain, a HP1 domain, or a MECP2 domain.
74. The method of any one of claim 46-72, wherein the epigenetic editor comprises from N terminus to C terminus the (i) a KRAB domain, a KAP1 domain, a HP1 domain, or a MECP2 domain, (ii) the DNA binding domain, and (iii) Dnmt3A-Dnmt3L fusion protein domain.
75. The method of claim 73 or 74, wherein the Dnmt3A-Dnmt3L fusion protein domain comprises from N terminus to C terminus: Dnmt3A-Dnmt3L.
76. The method of claim 73 or 74, wherein the Dnmt3A-Dnmt3L fusion protein domain comprises from N terminus to C terminus: Dnmt3L-Dnmt3A.
77. The method of any one of claims 46-76, wherein the epigenetic editor reduces expression of the target gene by at least 50% as compared to a wild-type expression level.
78. The method of any one of claims 46-77, wherein the reduction in expression of the target gene is maintained for at least 1 week, 4 weeks, 6 months, or 1 year.
79. The method of any one of claims 46-78, wherein the reduction in expression of the target gene is maintained in offspring cells derived from a cell comprising the target gene.
80. The method of any one of claims 1-45, wherein the epigenetic editor comprises an epigenetic effector domain that increases expression of the target gene as compared to a control gene in a cell not contacted with the epigenetic editor.
81. The method of claim 80, wherein the site-specific epigenetic modification or the epigenetic modification comprises DNA demethylation.
82. The method of claim 80 or 81, wherein the site-specific epigenetic modification or the epigenetic modification is in a CpG dinucleotide.
83. The method of claim 82, wherein the CpG dinucleotide is in a CpG island.
84. The method of claim 82, wherein the CpG dinucleotide is not in a CpG island.
85. The method of claim 83, wherein the site-specific epigenetic modification or the epigenetic modification comprises acetylation of the histone bound to the target gene.
86. The method of claim 80, wherein the site-specific epigenetic modification or the epigenetic modification comprises methylation of the histone bound to the target gene, optionally wherein the methylation of the histone is H3K4 methylation.
87. The method of claim 80, wherein the site-specific epigenetic modification comprises demethylation of the histone bound to the target gene, optionally wherein the demethylation of the histone is H3K9 demethylation.
88. The method of any one of claims 80-87, wherein the epigenetic effector domain comprises a DNA demethylase domain.
89. The method of claim 88, wherein the DNA demethylase domain comprises a TET family protein domain.
90. The method of claim 89, wherein the DNA demethylase domain comprises a TET1 protein.
91. The method of claim 88, wherein the epigenetic effector domain comprises a histone acetylase domain.
92. The method of any one of claims 80-87, wherein the epigenetic effector domain comprises a transcription activator, a DNA demethylase, a histone methyltransferase, a histone demethylase, a histone acetylase, or any combination thereof.
93. The method of any one of claims 80-87, wherein the epigenetic effector domain recruits a transcription activator, a DNA demethylase, a histone methyltransferase, a histone demethylase, a histone acetylase, or any combination thereof to the target gene.
94. The method of claim 92 or 93, wherein the epigenetic effector domain comprises a VP16 domain, a VP64 domain, a p65 domain, or a RTA domain.
95. The method of any one of claims 80-87, wherein the epigenetic effector domain comprises a protein from Table 5 or Table 6.
96. The method of any one of claims 80-95, wherein the epigenetic editor further comprises a second epigenetic effector domain that increases expression of the target gene.
97. The method of claim 96, wherein the second epigenetic effector domain comprises a DNA demethylase domain.
98. The method of claim 96, wherein the second epigenetic effector domain comprises a transcription activator, a DNA demethylase, a histone methyltransferase, a histone demethylase, a histone acetylase, or any combination thereof.
99. The method of claim 96, wherein the second epigenetic effector domain recruits a transcription activator, a DNA demethylase, a histone methyltransferase, a histone demethylase, a histone acetylase, or any combination thereof.
100. The method of claim 98 or 99, wherein the second epigenetic effector domain comprises a TET1 domain, a VP16 domain, a VP64 domain, a p65 domain, a RTA domain, or any combination thereof.
101. The method of claim 96, wherein the second epigenetic effector domain comprises a protein form Table 5 or Table 6.
102. The method of any one of claims 80-101, wherein the epigenetic editor comprises a DNA demethylase domain and a fusion of a VP64 domain, a p65 domain, and a RTA domain.
103. The method of any one of claim 80-102, wherein the epigenetic editor increases expression of the target gene by at least 50% as compared to a wild-type expression level.
104. The method of claim 80-103, wherein the increase in expression of the target gene expression is maintained for at least 1 week, 4 weeks, 6 months, or 1 year.
105. The method of any one of claims 80-104, wherein the increase in expression of the target gene is maintained in offspring cells derived from a cell comprising the target gene.
106. The method of any one of the preceding claims, wherein the epigenetic editor further comprises a second DNA binding domain that binds to a second target sequence in a second target gene, and wherein the DNA binding domain directs the epigenetic effector domain to effect an epigenetic modification in the second target gene or a histone bound to the second target gene.
107. The method of any one of claims 41-106, wherein the epigenetic editor further comprises a second guide polynucleotide that binds to the DNA binding domain and hybridizes with a second target sequence in a second target gene and directs the epigenetic editor to effect an epigenetic modification in the second target gene or a histone bound to the second target gene.
108. The method of claim 106 or 107, wherein the second target gene is the same as the target gene.
109. The method of claim 108, wherein the second target sequence overlaps with the target sequence.
110. The method of claim 108, wherein the second target sequence is within 1000 base pairs upstream or downstream of the target sequence.
111. The method of claim 108, wherein the second target sequence is within 500 base pairs upstream or downstream of the target sequence.
112. The method of claim 106 or 107, wherein the second target gene is different from the target gene.
113. The method of claim 112, wherein the target gene and the second target gene are associated with in a same metabolic pathway or function.
114. The method of claim 112, wherein the target gene and the second target gene are associated with a same disease or condition.
115. The method of any one of the preceding claims, wherein the epigenetic editor further comprises a linker.
116. The method of claim 115, wherein the linker is a peptide linker.
117. The method of claim 116, wherein the linker comprises an XTEN linker.
118. The method of any one of the preceding claims, wherein the contacting is ex vivo.
119. The method of any one of claims 1-114, wherein the contacting is in vivo in a subject.
120. The method of claim 119, wherein the subject is a human.
121. An epigenetically modified chromosome comprising a gene of interest (GOI), wherein at least 10% of all nucleotides or at least 10% of all histone tails bound with nucleotides within 200 base pairs upstream or downstream of an expression regulatory sequence of the GOI comprise an epigenetic modification as compared to an unmodified control chromosome comprising the gene of interest.
122. The epigenetically modified chromosome of claim 121, wherein at least 20% of all nucleotides within 200 base pairs upstream or downstream of the expression regulatory sequence comprise the epigenetic modification as compared to an unmodified control chromosome comprising the gene of interest.
123. The epigenetically modified chromosome of claim 121, wherein at least 50% of all nucleotides within 200 base pairs upstream or downstream of the expression regulatory sequence comprise the epigenetic modification as compared to an unmodified control chromosome comprising the gene of interest 124. The epigenetically modified chromosome of claim 121, wherein at least 10% of all nucleotides within 500 base pairs upstream or downstream of the expression regulatory sequence comprise the epigenetic modification as compared to an unmodified control chromosome comprising the gene of interest.
125. The epigenetically modified chromosome of claim 115, wherein the at least 20% of all nucleotides within 500 base pairs upstream or downstream of the expression regulatory sequence comprise the epigenetic modification as compared to an unmodified control chromosome comprising the gene of interest.
126. An epigenetically modified chromosome comprising a gene of interest (GOI), wherein at least 10% of all CpG dinucleotides within 200 base pairs upstream or downstream of an expression regulatory sequence of the GOI comprise an epigenetic modification as compared to an unmodified control chromosome comprising the gene of interest.
127. The epigenetically modified chromosome of claim 126, wherein at least 20% of all CpG dinucleotides within 200 base pairs upstream or downstream of the expression regulatory sequence comprise the epigenetic modification as compared to an unmodified control chromosome comprising the gene of interest.
128. The epigenetically modified chromosome of claim 126, wherein at least 50% of all CpG dinucleotides within 200 base pairs upstream or downstream of the expression regulatory sequence comprise the epigenetic modification as compared to an unmodified control chromosome comprising the gene of interest.
129. The epigenetically modified chromosome of claim 126, wherein at least 10% of all CpG dinucleotides within 500 base pairs upstream or downstream of the expression regulatory sequence comprise the epigenetic modification as compared to an unmodified control chromosome comprising the gene of interest.
130. The epigenetically modified chromosome of claim 126, wherein at least 20% of all CpG dinucleotides within 500 base pairs upstream or downstream of the expression regulatory sequence comprise the epigenetic modification as compared to an unmodified control chromosome comprising the gene of interest.
131. The epigenetically modified chromosome of any one of claims 126-130, wherein the CpG dinucleotides comprising the epigenetic modification are in a CpG island.
132. the epigenetically modified chromosome of any one of claims 126-130, wherein the CpG dinucleotides comprising the epigenetic modification are not in a CpG island.
133. The epigenetically modified chromosome of any one of claims 121-132, wherein the expression regulatory sequence comprises a promoter.
134. The epigenetically modified chromosome of any one of claims 121-132, wherein the expression regulatory sequence comprises a transcription start site.
135. The epigenetically modified chromosome of any one of claims 121-132, wherein the expression regulatory sequence comprises an enhancer.
136. The epigenetically modified chromosome of any one of claims 121-135, wherein the epigenetic modification is within a coding region of the GOI.
137. The epigenetically modified chromosome of any one of claims 121-136, wherein the target gene comprises an allele associated with a disease.
138. The epigenetically modified chromosome of any one of claims 121-136, wherein the target gene comprises two heterozygotic copies.
139. The epigenetically modified chromosome of any one of claims 121-137, wherein the target gene is heterozygous at an allele.
140. The epigenetically modified chromosome of claim 139, wherein the epigenetic modification is at one of the two heterozygotic copies and not the other.
141. The epigenetically modified chromosome of claim 140, wherein the epigenetic modification is at the heterozygotic allele.
142. The epigenetically modified chromosome of any one of claims 121-140, wherein the epigenetically modified chromosome is in a cell.
143. The epigenetically modified chromosome of claim 141, wherein the epigenetic modification results in reduced or silenced expression of the GOI as compared to the GOI in an unmodified control chromosome in a control cell.
144. The epigenetically modified chromosome of claim 143, wherein the epigenetic modification comprises DNA methylation.
145. The epigenetically modified chromosome of claim 143, wherein the epigenetic modification comprises de-acetylation of the histone tails.
146. The epigenetically modified chromosome of claim 143, wherein the site-specific epigenetic modification or the epigenetic modification comprises methylation of the histone bound to the target gene, optionally wherein the methylation of the histone is H3K9 methylation.
147. The epigenetically modified chromosome of claim 143, wherein the site-specific epigenetic modification comprises demethylation of the histone bound to the target gene, optionally wherein the demethylation of the histone is H3K4 demethylation.
148. The epigenetically modified chromosome of any one of claims 143-147, wherein the expression of the GOI is reduced by at least 50% as compared to a wild-type expression level.
149. The epigenetically modified chromosome of claim, wherein the reduction in expression of the GOI is maintained for at least 1 week, 4 weeks, 6 months, or 1 year.
150. The epigenetically modified chromosome any one of claims 143-149, wherein the reduction in expression of the GOI is maintained in offspring cells derived from the cell.
151. The epigenetically modified chromosome of claim 141, wherein the epigenetic modification results in increased expression of the GOI as compared to the GOI in an unmodified control chromosome in a control cell.
152. The epigenetically modified chromosome of claim 151, wherein the epigenetic modification comprises DNA demethylation.
153. The epigenetically modified chromosome of claim 151, wherein the epigenetic modification comprises acetylation of the histone tails.
154. The epigenetically modified chromosome of claim 151, wherein the epigenetic modification comprises methylation of the histone tails, optionally wherein the methylation of the histone is H3K4 methylation.
155. The epigenetically modified chromosome of claim 151, wherein the epigenetic modification comprises demethylation of the histone tails, optionally wherein the demethylation of the histone is H3K9 demethylation.
156. The epigenetically modified chromosome any one of claims 151-155, wherein the expression of the GOI is increased by at least 50% as compared to a wild-type expression level.
157. The epigenetically modified chromosome any one of claims 151-156, wherein the increase in expression of the GOI is maintained for at least 1 week, 4 weeks, 6 months, or 1 year.
158. The epigenetically modified chromosome of any one of claims 151-157, wherein the increase in expression of the GOI is maintained in offspring cells derived from the cell.
159. A cell comprising the epigenetically modified chromosome of any one of claims 121-158.
160. The cell of claim 159, wherein the cell is a non-dividing cell.
161. The cell of claim 159, wherein the cell is a primary cell.
162. The cell of claim 159, wherein the cell is a mammalian cell.
163. The cell of claim 159, wherein the cell is a human cell.
164. The epigenetically modified chromosome of any one of claims 121-158, wherein the epigenetically modified chromosome is in a subject.
165. The epigenetically modified chromosome of claim 164, wherein the subject is a human.
166. An epigenetic editor that comprises a DNA binding domain, a DNA methylation regulatory protein, and an affinity domain, wherein the DNA binding domain binds to a target sequence in a target chromosome comprising a target gene, wherein the affinity domain specifically binds to an epigenetic effector protein in a cell comprising the target gene and directs the epigenetic effector protein to the target gene to effect an epigenetic modification in a nucleotide in the target gene or a histone bound to the target gene when contacted with the target chromosome.
167. An epigenetic editor that comprises a DNA binding domain, an epigenetic effector protein, and an affinity domain, wherein the DNA binding domain binds to a target sequence in a target chromosome comprising a target gene, wherein the affinity domain specifically binds to a DNA methylation regulatory protein in a cell comprising the target gene and directs the DNA methylation regulatory protein to the target gene to effect an epigenetic modification in a nucleotide in the target gene.
168. The epigenetic editor of claim 166 or 167, wherein the DNA methylation regulatory protein comprises a DNA methyltransferase domain.
169. The epigenetic editor of claim 168, wherein the DNA methyltransferase domain comprises a Dnmt1 domain, a Dnmt3A domain, a Dnmt3L domain, or a Dnmt3B domain.
170. The epigenetic editor of claim 168, wherein the DNA methyltransferase domain comprises a Dnmt3A-Dnmt3L fusion.
171. The epigenetic editor of any one of claims 166-170, wherein the epigenetic effector protein results in decreased or silenced expression of the target gene as compared to the target gene not contacted with the epigenetic editor.
172. The epigenetic editor of any one of claims 166-171, wherein the epigenetic effector protein comprises a histone deacetylase.
173. The epigenetic editor of any one of claims 166-171, wherein the epigenetic effector protein comprises a transcription repressor, a DNA methyltransferase, a histone methyltransferase, a histone demethylase, a histone deacetylase, or any combination thereof.
174. The epigenetic editor of any one of claims 166-171, wherein the epigenetic effector protein recruits a transcription repressor, a DNA methyltransferase, a histone methyltransferase, a histone demethylase, a histone deacetylase, or any combination thereof in the cell to the target gene.
175. The epigenetic editor of any one of claims 166-171, wherein the epigenetic effector protein comprises a KRAB protein, a KAP1 protein, a MECP2 protein, or a HP1 protein.
176. The epigenetic editor of any one of claims 166-171, wherein the epigenetic effector protein comprises a protein from Table 2 or Table 3.
177. The epigenetic editor of any one of claim 166 or 168-175, wherein the epigenetic editor comprises a Dnmt3A-Dnm3L fusion protein domain and the affinity domain that specifically binds to KAP1.
178. The epigenetic editor of any one of claim 166 or 168-175, wherein the epigenetic editor comprises a Dnmt3A-Dnm3L fusion protein domain and the affinity domain that specifically binds to KRAB.
179. The epigenetic editor of any one of claim 166 or 168-175, wherein the epigenetic editor comprises a Dnmt3A-Dnm3L fusion protein domain and the affinity domain that specifically binds to MECP2.
180. The epigenetic editor of any one of claim 166 or 168-175, wherein the epigenetic editor comprises a Dnmt3A-Dnm3L fusion protein domain and the affinity domain that specifically binds to HP1.
181. The epigenetic editor of any one of claim 166 or 168-175, wherein the epigenetic editor comprises a Dnmt3A-Dnm3L fusion protein domain and the affinity domain that specifically binds to a chromoshadow domain.
182. The epigenetic editor of any one of claims 177-181, wherein the epigenetic editor comprises from N terminus to C terminus: (i) the Dnmt3A-Dnmt3L fusion protein domain, (ii) the DNA binding domain, and (iii) the affinity domain.
183. The epigenetic editor of any one of claims 177-181, wherein the epigenetic editor comprises from N terminus to C terminus (i) the affinity domain, (ii) the DNA binding domain, and (iii) the Dnmt3A-Dnmt3L fusion protein domain.
184. The epigenetic editor of any one of claims 177-183, wherein the Dnmt3A-Dnmt3L fusion protein domain comprises from N terminus to C terminus: Dnmt3A-Dnmt3L.
185. The epigenetic editor of any one of claims 177-183, wherein the Dnmt3A-Dnmt3L fusion protein domain comprises from N terminus to C terminus: Dnmt3L-Dnmt3A.
186. The epigenetic editor of any one of claims 167-175, wherein the epigenetic effector protein comprises a histone deacetylase domain and the affinity domain specifically binds to a Dnmt3A domain.
187. The epigenetic editor of any one of claims 167-175, wherein the epigenetic effector protein comprises a histone deacetylase domain and the affinity domain specifically binds to a Dnmt3L domain.
188. The epigenetic editor of any one of claims 167-175, wherein the epigenetic effector protein comprises a histone deacetylase domain and the affinity domain specifically binds to a Dnmt3B domain.
189. The epigenetic editor of any one of claims 167-175, wherein the epigenetic effector protein comprises a histone deacetylase domain and the affinity domain specifically binds to a Dnmt1 domain.
190. The epigenetic editor of any one of claims 167-175, wherein the epigenetic effector protein comprises a KAP1 domain and the affinity domain that specifically binds to a Dnmt3A domain, a Dnmt3L domain, a Dnmt3B domain, or a Dnmt1 domain.
191. The epigenetic editor of any one of claims 167-175, wherein the epigenetic effector protein comprises a KRAB domain and the affinity domain that specifically binds to a Dnmt3A domain, a Dnmt3L domain, a Dnmt3B domain, or a Dnmt1 domain.
192. The epigenetic editor of any one of claims 167-175, wherein the epigenetic effector protein comprises a MECP2 domain and the affinity domain that specifically binds to a Dnmt3A domain, a Dnmt3L domain, a Dnmt3B domain, or a Dnmt1 domain.
193. The epigenetic editor of any one of claims 167-175, wherein the epigenetic effector protein comprises a HP1 domain and the affinity domain that specifically binds to a Dnmt3A domain, a Dnmt3L domain, a Dnmt3B domain, or a Dnmt1 domain.
194. The epigenetic editor of any one of claims 167-175, wherein the epigenetic effector protein comprises a chromoshadow domain and an affinity domain that specifically binds to a Dnmt3A domain, a Dnmt3L domain, a Dnmt3B domain, or a Dnmt1 domain.
195. The epigenetic editor of any one of claims 167-175, wherein the epigenetic editor comprises from N terminus to C terminus: (i) a KAP1 domain, a KRAB domain, a HP1 domain, a MECP2 domain, or a chromoshadow domain, (ii) the DNA binding domain, and (iii) the affinity domain.
196. The epigenetic editor of any one of claims 167-175, wherein the epigenetic editor comprises from N terminus to C terminus (i) the affinity domain, (ii) the DNA binding domain, and (iii) (i) a KAP1 domain, a KRAB domain, a HP1 domain, a MECP2 domain, or a chromoshadow domain.
197. The epigenetic editor of any one of claim 166 or 168-175, wherein the epigenetic editor further comprises a second affinity domain that specifically binds to a second epigenetic effector protein in the cell, wherein the second epigenetic effector protein results in reduced or silenced expression of the target gene.
198. The epigenetic editor of claim 197, wherein the second effector protein comprises a DNA methyltransferase domain.
199. The epigenetic editor of claim 197, wherein the second epigenetic effector protein comprises a transcription repressor, a DNA methyltransferase, a histone methyltransferase, a histone demethylase, a histone deacetylase, or any combination thereof.
200. The epigenetic editor of claim 197, wherein the second epigenetic effector protein recruits a transcription repressor, a DNA methyltransferase, a histone methyltransferase, a histone demethylase, a histone deacetylase, or any combination thereof to the target gene.
201. The epigenetic editor of claim 197, wherein the second epigenetic effector protein comprises a KRAB domain, a KAP1 domain, a HP1 domain, a Dnmt3A domain, a Dnmt3L domain, a chromoshadow domain, or any combination thereof.
202. The epigenetic editor of claim 197, wherein the second epigenetic effector domain comprises a protein of Table 2 or Table 3.
203. The epigenetic editor of claim 166 or 167, wherein the DNA methylation regulatory protein comprises a DNA demethylase domain.
204. The epigenetic editor of claim 203, wherein the DNA demethylase domain comprise a TET family protein.
205. The epigenetic editor of claim 204, wherein the DNA demethylase domain comprise TET1.
206. The epigenetic editor of any one of claims 203-205, wherein the epigenetic effector protein results in increased expression of the target gene as compared to the target gene not contacted with the epigenetic editor.
207. The epigenetic editor of any one of claims 203-206, wherein the epigenetic effector protein comprises a histone acetyltransferase.
208. The epigenetic editor of any one of claims 203-206, wherein the epigenetic effector protein recruits a transcription activator, a DNA demethylase, a histone methyltransferase, a histone demethylase, a histone acetylase, or any combination thereof to the target gene.
209. The epigenetic editor of any one of claims 203-206, wherein the epigenetic effector protein comprises a VP16 domain, a VP64 domain, a p65 domain, or a RTA domain.
210. The epigenetic editor of any one of claims 203-206, wherein the epigenetic effector protein comprises a protein from Table 5 or Table 6.
211. The epigenetic editor of any one of claims 203-210, wherein the epigenetic editor further comprises a second affinity domain that specifically binds to a second epigenetic effector protein that increases expression of the target gene.
212. The epigenetic editor of claim 211, wherein the second epigenetic effector protein comprises a DNA demethylase domain.
213. The epigenetic editor of claim 211, wherein the second epigenetic effector protein comprises a histone acetyltransferase domain.
214. The epigenetic editor of claim 211, wherein the second epigenetic effector protein recruits a transcription activator, a DNA demethylase, a histone methyltransferase, a histone demethylase, a histone acetylase, or any combination thereof.
215. The epigenetic editor of claim 211, wherein the second epigenetic effector protein comprises a TET1 domain, a VP16 domain, a VP64 domain, a p65 domain, a RTA domain, or any combination thereof.
216. The epigenetic editor of claim 211, wherein the second epigenetic effector protein comprises a protein form Table 5 or Table 6.
217. The epigenetic editor of any one of claims 166-216, wherein the affinity domain comprises a single chain antibody, a nanobody, an antigen binding sequence, an antibody, a nanobody, a functional antibody fragment, a single chain variable fragment (scFv), an Fab, a single-domain antibody (sdAb), a VH domain, a VL domain, a VNAR domain, a VHH domain, a bispecific antibody, a diabody, or a functional fragment or a combination thereof.
218. An epigenetic editor that comprises a DNA binding domain, a DNA methyltransferase domain, and an epigenetic effector domain, wherein the epigenetic effector domain is a KAP1 domain, a HP1 domain, a chromoshadow domain, or a MECP2 domain.
219. An epigenetic editor that comprises a DNA binding domain, a DNA methyltransferase domain selected from Table 1, and an epigenetic effector domain selected from Table 2 or Table 3.
220. An epigenetic editor that comprises a DNA binding domain, a DNA demethylase domain selected from Table 4, and an epigenetic effector domain selected from Table 5 or Table 6.
221. The epigenetic editor of any one of claim 218 or 220, wherein the DNA methyltransferase domain comprises a Dnmt1 domain, a Dnmt3A domain, a Dnmt3L domain, or a Dnmt3B domain.
222. The epigenetic editor of any one of claim 218 or 220, wherein the DNA methyltransferase domain comprises a Dnmt3A-Dnmt3L fusion.
223. The epigenetic editor of claim 222, wherein the Dnmt3A-Dnmt3L fusion protein domain comprises from N terminus to C terminus: Dnmt3A-Dnmt3L.
224. The epigenetic editor of claim 222, wherein the Dnmt3A-Dnmt3L fusion protein domain comprises from N terminus to C terminus: Dnmt3L-Dnmt3A.
225. The epigenetic editor of any one of claims 222-224, comprising from N terminus to C terminus (i) the Dnmt3A-Dnmt3L fusion protein domain, (ii) the DNA binding domain, and (iii) epigenetic effector domain.
226. The epigenetic editor of any one of claims 222-225, comprising from N terminus to C terminus (i) the epigenetic effector domain, (ii) the DNA binding domain, and (iii) Dnmt3A-Dnmt3L fusion protein domain.
227. The epigenetic editor of any one of claims 218-226, wherein the DNA binding domain binds to a target sequence in a target gene and directs the epigenetic effector domain to the target gene to effect an epigenetic modification in a nucleotide in the target gene or a histone bound to the target gene when contacted with the target gene.
228. The method of any one of claim 227, wherein the epigenetic effector domain results in reduced or silenced expression of the target gene as compared to the target gene not contacted with the epigenetic editor.
229. The method of any one of claim 227, wherein the epigenetic effector domain results in increased expression of the target gene as compared to the target gene not contacted with the epigenetic editor.
230. The epigenetic editor of any one of claims 166-229, wherein the epigenetic modification is within a coding region of the target gene.
231. The epigenetic editor of any one of claims 166-229, wherein the epigenetic modification is in an expression regulatory sequence of the target gene.
232. The epigenetic editor of any one of claim 166-229, wherein the epigenetic modification is within 3000 base pairs upstream or downstream of an expression regulatory sequence of the target gene.
233. The epigenetic editor of claim 231 or 232, wherein the expression regulatory sequence comprises a promoter.
234. The epigenetic editor of claim 231 or 232, wherein the expression regulatory sequence comprises a transcription initiation start site.
235. The epigenetic editor of claim 231 or 232, wherein the expression regulatory sequence comprises an enhancer.
236. The method of any one of claim 219 or 221-235, wherein the epigenetic editor further comprises a second epigenetic effector domain that results in reduced or silenced expression of the target gene.
237. The method of claim 236, wherein the second epigenetic effector domain comprises or recruits a transcription repressor, a DNA methyltransferase, a histone methyltransferase, a histone demethylase, a histone deacetylase, or any combination thereof.
238. The method of claim 236, wherein the second epigenetic effector domain comprises a protein of Table 2 or Table 3.
239. The method of any one of claims 232-238, wherein the epigenetic effector domain and the second epigenetic effector domain synergistically reduces or silences expression of the target gene.
240. The method of any one of claim 218 or 220-234, wherein the epigenetic editor further comprises a second epigenetic effector domain that results in increased expression of the target gene.
241. The method of claim 240, wherein the second epigenetic effector domain comprises a transcription activator, a DNA demethylase, a histone methyltransferase, a histone demethylase, a histone acetyltransferase, or any combination thereof.
242. The method of claim 240, wherein the second epigenetic effector domain recruits a transcription activator, a DNA demethylase, a histone methyltransferase, a histone demethylase, a histone acetyltransferase, or any combination thereof to the target gene.
243. The method of claim 240, wherein the second epigenetic effector domain comprises a protein of table 5 or Table 6.
244. The method of any one of claims 241-243, wherein the epigenetic effector domain and the second epigenetic effector domain synergistically reduces or silences expression of the target gene.
245. The epigenetic editor of any one of claims 166-244, wherein the target gene comprises an allele associated with a disease.
246. The epigenetic editor of any one of claims 166-244, wherein the target gene comprises two heterozygotic copies and wherein the DNA binding domain binds to one of the two heterozygotic copies and not the other.
247. The epigenetic editor of any one of claims 166-244, wherein the target gene is heterozygous at an allele.
248. The epigenetic editor of any one of claims 166-247, wherein the DNA binding domain comprises a zinc finger motif.
249. The epigenetic editor of any one of claims 166-248, wherein the DNA binding domain comprises a zinc finger array.
250. The epigenetic editor of claim 249, wherein the zinc finger array comprises at least six zinc fingers.
251. The epigenetic editor of claim 249, wherein the zinc finger array comprises at least three subsets of zinc fingers each comprising at least two zinc fingers.
252. The epigenetic editor of any one of claims 166-247, wherein the DNA binding domain comprises a nucleic acid guided DNA binding domain bound to a guide polynucleotide.
253. The epigenetic editor of claim 252, wherein the DNA binding domain comprises CRISPR-Cas protein bound to the guide polynucleotide.
254. The epigenetic editor of claim 252, wherein the guide polynucleotide hybridizes with the target sequence.
255. The epigenetic editor of claim 253 or 254, wherein the CRISPR-Cas protein comprises a nuclease inactive Cas9 (dCas9).
256. The epigenetic editor of claim 253 or 254, wherein the CRISRP-Cas protein comprises a nuclease inactive Cas12a (dCas12a).
257. The epigenetic editor of claim 237 or 238, wherein the CRISRP-Cas protein comprises a nuclease inactive CasX (dCasX).
258. The epigenetic editor of any one of claims 248-257, wherein the epigenetic editor further comprises a second DNA binding domain that binds to a second target sequence in a second target gene, and wherein the second DNA binding domain directs the epigenetic effector domain to effect an epigenetic modification in the second target gene or a histone bound to the second target gene.
259. The epigenetic editor of claim 258, wherein the second DNA binding domain comprises a zinc finger array.
260. The epigenetic editor of claim 259, wherein the zinc finger array comprises at least six zinc fingers.
261. The epigenetic editor of claim 259, wherein the zinc finger array comprises at least three subsets of zinc fingers each comprising at least two zinc fingers.
262. The epigenetic editor of claim 258, wherein the second DNA binding domain comprises a second nucleic acid guided DNA binding domain bound to a second guide polynucleotide.
263. The epigenetic editor of claim 262, wherein the second guide polynucleotide hybridizes with the second target sequence in the second target gene.
264. The method of any one of claims 258-263, wherein the second target gene is the same as the target gene.
265. The method of claim 264, wherein the second target sequence overlaps with the target sequence.
266. The method of claim 264 or 265, wherein the second target sequence is within 1000 base pairs flanking the target sequence.
267. The method of claim 264 or 265, wherein the second target sequence is within 500 base pairs flanking the target sequence.
268. The method of any one of claims 258-263, wherein the second target gene is different from the target gene.
269. The method of claim 268, wherein the target gene and the second target gene are associated with in a same metabolic pathway or function.
270. The method of claim 268, wherein the target gene and the second target gene are associated with a same disease or condition.
271. The epigenetic editor of any one of claims 166-270, wherein the epigenetic editor further comprises a linker.
272. The epigenetic editor of claim 271, wherein the linker is a peptide linker, thereby forming a fusion protein.
273. A nucleic acid encoding the fusion protein of claim 272.
274. A set of nucleic acids comprising a first nucleic acid encoding a first part and a second nucleic acid encoding a second part of the fusion protein of claim 272, wherein the first part and the second part comprise the fusion protein of claim 272 when combined.
275. The set of nucleic acids of claim 274, wherein the first nucleic acid further encodes a N terminal part of an intein and wherein the second nucleic acid further comprises a C terminal part of the intein.
276. A vector comprising the nucleic acid of claim 273.
277. A set of vectors comprising a first vector comprising the first nucleic acid of claim 274 and a second vector comprising the second nucleic acid of claim 274.
278. The vector of claim 276, wherein the vector is a virus vector.
279. The vector of claim 278, wherein the vector is a lentivirus vector, an adenovirus vector, a herpes virus vector, or an adeno-associated virus (AAV) vector.
280. The vector of claim 279, wherein the vector is an AAV1, AAV2, AAV3, AAV4, AAV5, AAV6, AAV7, AAV8, AAV9, or AAV10 vector.
281. The set of vectors of claim 277, wherein the first vector and the second vector are virus vectors.
282. The set of vectors of claim 277, wherein the first vector and the second vector are lentivirus vectors, adenovirus vectors, herpes virus vectors, or adeno-associated virus (AAV) vectors.
283. The vector of claim 279, wherein the first vector or the second vector is an AAV1, AAV2, AAV3, AAV4, AAV5, AAV6, AAV7, AAV8, AAV9, or AAV10 vector.
284. A cell comprising the epigenetic editor of any one of claims 161-272, the nucleic acid of claim 273, the set of nucleic acids of claim 274 or 275, the vector of any one of claim 276 or 278-280, or the set of vectors of any one of claim 277 or 281-283.
285. The cell of any one of claims 159-163 or 284, wherein the cell is a primary cell.
286. The cell of any one of claims 159-163 or 284, wherein the cell is a non-dividing cell.
287. The cell of any one of claims 159-163 or 284, wherein the cell is a stem cell.
288. The cell of any one of claims 159-163 or 284-287, wherein the cell is a mammalian cell.
289. The cell of claim 288, wherein the cell is a human cell.
290. The cell of any one of claims 285-289, wherein the cell is ex vivo or in vivo.
291. A composition comprising the epigenetic editor of any one of claims 161-272, the nucleic acid of claim 273, the set of nucleic acids of claim 274 or 275, the vector of any one of claim 276 or 278-280, the set of vectors of any one of claim 277 or 281-283, or the cell of any one of claims 284-290.
292. The composition of claim 291, further comprising a pharmaceutically acceptable carrier.
293. An Epigenetic Editor comprising:
   a DNA binding domain capable of binding to a target sequence in a target chromosome and directing the Epigenetic Editor to repress or silence expression of a target gene;
   one or more effector domains selected from the group consisting of a DNA methyltransferase domain and an effector domain that recruits a DNA methyltransferase; and
   one or more effector domains selected from the group consisting of a histone methyltransferase domain that reduces transcription at the target gene, a histone demethylase domain that reduces transcription at the target gene, a histone deacetylase domain, an effector domain that recruits a histone methyltransferase that reduces transcription at the target gene, an effector domain that recruits a histone demethylase that reduces transcription at the target gene and an effector domain that recruits a histone deacetylase.
294. The Epigenetic Editor of claim 293, wherein the Epigenetic Editor further comprises one or more effector domains selected from the group consisting of a transcription repressor domain and an effector domain that recruits a transcriptional repressor.
295. The Epigenetic Editor of claim 294, wherein the transcriptional repressor domain or the effector domain that recruits a transcriptional repressor is not an effector domain from claims 293 (c).
296. The Epigenetic Editor of claims 293-295, wherein the effector domain from (c) is a KRAB repression domain.
297. The Epigenetic Editor of claim 296, wherein the KRAB repression domain is a KOX1/ZNF10 domain or a ZIM3 domain.
298. An Epigenetic Editor comprising:
   a DNA binding domain capable of binding to a target sequence in a target chromosome and directing the Epigenetic Editor to increase expression of a target gene;
   one or more effector domains selected from the group consisting of a DNA demethylase domain and an effector domain that recruits a DNA demethylase; and
   one or more effector domains selected from the group consisting of a histone methyltransferase domain that increases transcription at the target gene, a histone demethylase domain that increases transcription at the target gene, a histone acetylase domain, an effector domain that recruits a histone methyltransferase that increases transcription at the target gene, an effector domain that recruits a histone demethylase that increases transcription at the target gene and an effector domain that recruits a histone acetylase.
299. The Epigenetic Editor of claim 298, wherein the Epigenetic Editor further comprises one or more effector domains selected from the group consisting of a transcription activation domain and an effector domain that recruits a transcription activator.
300. The Epigenic Editor of claim 299, wherein the selected effector domain is not an effector domain from claim 298 (c).
301. The Epigenetic Editor of claim 300, wherein the selected effector domain is a VP16 domain, a VP64 domain, a p65 domain, or ab RTA domain.
302. The Epigenetic Editor of claims 293-301, wherein the Epigenetic Editor is a polypeptide.

Sequence Tables

| SEQ | | |
|---|---|---|
| ID | | |
| NO | Description | Sequence |
| 1 | S. | ATGGATAAGAAATACTCAATAGGCTTAGATATCGGCACAAATAGCGTCG |
| | pyogenes | GATGGGCGGTGATCACTGATGAATATAAGGTTCCGTCTAAAAAGTTCAA |
| | WT Cas9 | GGTTCTGGGAAATACAGACCGCCACAGTATCAAAAAAAATCTTATAGGG |
| | NT | GCTCTTTTATTTGACAGTGGAGAGACAGCGGAAGCGACTCGTCTCAAAC |
| | Sequence | GGACAGCTCGTAGAAGGTATACACGTCGGAAGAATCGTATTTGTTATCT |
| | | ACAGGAGATTTTTTCAAATGAGATGGCGAAAGTAGATGATAGTTTCTTT |
| | | CATCGACTTGAAGAGTCTTTTTTGGTGGAAGAAGACAAGAAGCATGAAC |
| | | GTCATCCTATTTTTGGAAATATAGTAGATGAAGTTGCTTATCATGAGAA |
| | | ATATCCAACTATCTATCATCTGCGAAAAAAATTGGTAGATTCTACTGAT |
| | | AAAGCGGATTTGCGCTTAATCTATTTGGCCTTAGCGCATATGATTAAGTT |
| | | TCGTGGTCATTTTTTGATTGAGGGAGATTTAAATCCTGATAATAGTGATG |
| | | TGGACAAACTATTTATCCAGTTGGTACAAACCTACAATCAATTATTTGA |
| | | AGAAAACCCTATTAACGCAAGTGGAGTAGATGCTAAAGCGATTCTTTCT |
| | | GCACGATTGAGTAAATCAAGACGATTAGAAAATCTCATTGCTCAGCTCC |
| | | CCGGTGAGAAGAAAAATGGCTTATTTGGGAATCTCATTGCTTTGTCATT |
| | | GGGTTTGACCCCTAATTTTAAATCAAATTTTGATTTGGCAGAAGATGCTA |
| | | AATTACAGCTTTCAAAAGATACTTACGATGATGATTTAGATAATTTATTG |
| | | GCGCAAATTGGAGATCAATATGCTGATTTGTTTTTGGCAGCTAAGAATTT |
| | | ATCAGATGCTATTTTACTTTCAGATATCCTAAGAGTAAATACTGAAATA |
| | | ACTAAGGCTCCCCTATCAGCTTCAATGATTAAACGCTACGATGAACATC |
| | | ATCAAGACTTGACTCTTTTAAAAGCTTTAGTTCGACAACAACTTCCAGA |
| | | AAAGTATAAAGAAATCTTTTTTGATCAATCAAAAAACGGATATGCAGGT |
| | | TATATTGATGGGGGAGCTAGCCAAGAAGAATTTTATAAATTTATCAAAC |
| | | CAATTTTAGAAAAAATGGATGGTACTGAGGAATTATTGGTGAAACTAAA |
| | | TCGTGAAGATTTGCTGCGCAAGCAACGGACCTTTGACAACGGCTCTATT |
| | | CCCCATCAAATTCACTTGGGTGAGCTGCATGCTATTTTGAGAAGACAAG |
| | | AAGACTTTTATCCATTTTTAAAAGACAATCGTGAGAAGATTGAAAAAAT |
| | | CTTGACTTTTCGAATTCCTTATTATGTTGGTCCATTGGCGCGTGGCAATA |
| | | GTCGTTTTGCATGGATGACTCGGAAGTCTGAAGAAACAATTACCCCATG |
| | | GAATTTTGAAGAAGTTGTCGATAAAGGTGCTTCAGCTCAATCATTTATTG |
| | | AACGCATGACAAACTTTGATAAAAATCTTCCAAATGAAAAAGTACTACC |
| | | AAAACATAGTTTGCTTTATGAGTATTTTACGGTTTATAACGAATTGACAA |
| | | AGGTCAAATATGTTACTGAAGGAATGCGAAAACCAGCATTTCTTTCAGG |
| | | TGAACAGAAGAAAGCCATTGTTGATTTACTCTTCAAAACAAATCGAAAA |
| | | GTAACCGTTAAGCAATTAAAAGAAGATTATTTCAAAAAAATAGAATGTT |
| | | TTGATAGTGTTGAAATTTCAGGAGTTGAAGATAGATTTAATGCTTCATTA |
| | | GGTACCTACCATGATTTGCTAAAAATTATTAAAGATAAAGATTTTTTGG |
| | | ATAATGAAGAAAATGAAGATATCTTAGAGGATATTGTTTTAACATTGAC |
| | | CTTATTTGAAGATAGGGAGATGATTGAGGAAAGACTTAAAACATATGCT |
| | | CACCTCTTTGATGATAAGGTGATGAAACAGCTTAAACGTCGCCGTTATA |
| | | CTGGTTGGGGACGTTTGTCTCGAAAATTGATTAATGGTATTAGGGATAA |
| | | GCAATCTGGCAAAACAATATTAGATTTTTTGAAATCAGATGGTTTTGCC |
| | | AATCGCAATTTTATGCAGCTGATCCATGATGATAGTTTGACATTTAAAG |
| | | AAGACATTCAAAAAGCACAAGTGTCTGGACAAGGCGATAGTTTACATG |
| | | AACATATTGCAAATTTAGCTGGTAGCCCTGCTATTAAAAAAGGTATTTT |
| | | ACAGACTGTAAAAGTTGTTGATGAATTGGTCAAAGTAATGGGGGGCAT |
| | | AAGCCAGAAAATATCGTTATTGAAATGGCACGTGAAAATCAGACAACTC |
| | | AAAAGGGCCAGAAAAATTCGCGAGAGCGTATGAAACGAATCGAAGAAG |
| | | GTATCAAAGAATTAGGAAGTCAGATTCTTAAAGAGCATCCTGTTGAAAA |
| | | TACTCAATTGCAAAATGAAAAGCTCTATCTCTATTATCTCCAAAATGGA |
| | | AGAGACATGTATGTGGACCAAGAATTAGATATTAATCGTTTAAGTGATT |
| | | ATGATGTCGATCACATTGTTCCACAAAGTTTCCTTAAAGACGATTCAATA |
| | | GACAATAAGGTCTTAACGCGTTCTGATAAAAATCGTGGTAAATCGGATA |
| | | ACGTTCCAAGTGAAGAAGTAGTCAAAAAGATGAAAAACTATTGGAGAC |
| | | AACTTCTAAACGCCAAGTTAATCACTCAACGTAAGTTTGATAATTTAAC |
| | | GAAAGCTGAACGTGGAGGTTTGAGTGAACTTGATAAAGCTGGTTTTATC |
| | | AAACGCCAATTGGTTGAAACTCGCCAAATCACTAAGCATGTGGCACAAA |
| | | TTTTGGATAGTCGCATGAATACTAAATACGATGAAAATGATAAACTTAT |
| | | TCGAGAGGTTAAAGTGATTACCTTAAAATCTAAATTAGTTTCTGACTTCC |
| | | GAAAAGATTTCCAATTCTATAAAGTACGTGAGATTAACAATTACCATCA |
| | | TGCCCATGATGCGTATCTAAATGCCGTCGTTGGAACTGCTTTGATTAAGA |
| | | AATATCCAAAACTTGAATCGGAGTTTGTCTATGGTGATTATAAAGTTTAT |
| | | GATGTTCGTAAAATGATTGCTAAGTCTGAGCAAGAAATAGGCAAAGCA |
| | | ACCGCAAAATATTTCTTTTACTCTAATATCATGAACTTCTTCAAAACAGA |
| | | AATTACACTTGCAAATGGAGAGATTCGCAAACGCCCTCTAATCGAAACT |
| | | AATGGGGAAACTGGAGAAATTGTCTGGGATAAAGGGCGAGATTTTGCC |
| | | ACAGTGCGCAAAGTATTGTCCATGCCCCAAGTCAATATTGTCAAGAAAA |
| | | CAGAAGTACAGACAGGCGGATTCTCCAAGGAGTCAATTTTACCAAAAA |
| | | GAAATTCGGACAAGCTTATTGCTCGTAAAAAAGACTGGGATCCAAAAA |
| | | AATATGGTGGTTTTGATAGTCCAACGGTAGCTTATTCAGTCCTAGTGGTT |
| | | GCTAAGGTGGAAAAAGGGAAATCGAAGAAGTTAAAATCCGTTAAAGAG |
| | | TTACTAGGGATCACAATTATGGAAAGAAGTTCCTTTGAAAAAAATCCGA |
| | | TTGACTTTTTAGAAGCTAAAGGATATAAGGAAGTTAAAAAAGACTTAAT |
| | | CATTAAACTACCTAAATATAGTCTTTTTGAGTTAGAAAACGGTCGTAAA |
| | | CGGATGCTGGCTAGTGCCGGAGAATTACAAAAAGGAAATGAGCTGGCT |
| | | CTGCCAAGCAAATATGTGAATTTTTTATATTTAGCTAGTCATTATGAAAA |
| | | GTTGAAGGGTAGTCCAGAAGATAACGAACAAAAACAATTGTTTGTGGA |
| | | GCAGCATAAGCATTATTTAGATGAGATTATTGAGCAAATCAGTGAATTT |
| | | TCTAAGCGTGTTATTTTAGCAGATGCCAATTTAGATAAAGTTCTTAGTGC |
| | | ATATAACAAACATAGAGACAAACCAATACGTGAACAAGCAGAAAATAT |
| | | TATTCATTTATTTACGTTGACGAATCTTGGAGCTCCCGCTGCTTTTAAAT |
| | | ATTTTGATACAACAATTGATCGTAAACGATATACGTCTACAAAAGAAGT |
| | | TTTAGATGCCACTCTTATCCATCAATCCATCACTGGTCTTTATGAAACAC |
| | | GCATTGATTTGAGTCAGCTAGGAGGTGACTGA |
| 2 | S. | MDKKYSIGLDIGTNSVGWAVITDEYKVPSKKFKVLGNTDRHSIKKNLIGAL |
| | pyogenes | LFDSGETAEATRLKRTARRRYTRRKNRICYLQEIFSNEMAKVDDSFFHRLEE |
| | WT Cas9 | SFLVEEDKKHERHPIFGNIVDEVAYHEKYPTIYHLRKKLVDSTDKADLRLIY |
| | AA | LALAHMIKFRGHFLIEGDLNPDNSDVDKLFIQLVQTYNQLFEENPINASGVD |
| | Sequence | AKAILSARLSKSRRLENLIAQLPGEKKNGLFGNLIALSLGLTPNFKSNFDLAE |
| | | DAKLQLSKDTYDDDLDNLLAQIGDQYADLFLAAKNLSDAILLSDILRVNTEI |
| | | TKAPLSASMIKRYDEHHQDLTLLKALVRQQLPEKYKEIFFDQSKNGYAGYI |
| | | DGGASQEEFYKFIKPILEKMDGTEELLVKLNREDLLRKQRTFDNGSIPHQIH |
| | | LGELHAILRRQEDFYPFLKDNREKIEKILTFRIPYYVGPLARGNSRFAWMTR |
| | | KSEETITPWNFEEVVDKGASAQSFIERMTNFDKNLPNEKVLPKHSLLYEYFT |
| | | VYNELTKVKYVTEGMRKPAFLSGEQKKAIVDLLFKTNRKVTVKQLKEDYF |
| | | KKIECFDSVEISGVEDRFNASLGTYHDLLKIIKDKDFLDNEENEDILEDIVLTL |
| | | TLFEDREMIEERLKTYAHLFDDKVMKQLKRRRYTGWGRLSRKLINGIRDK |
| | | QSGKTILDFLKSDGFANRNFMQLIHDDSLTFKEDIQKAQVSGQGDSLHEHIA |
| | | NLAGSPAIKKGILQTVKVVDELVKVMGRHKPENIVIEMARENQTTQKGQK |
| | | NSRERMKRIEEGIKELGSQILKEHPVENTQLQNEKLYLYYLQNGRDMYVDQ |
| | | ELDINRLSDYDVDHIVPQSFLKDDSIDNKVLTRSDKNRGKSDNVPSEEVVK |
| | | KMKNYWRQLLNAKLITQRKFDNLTKAERGGLSELDKAGFIKRQLVETRQIT |
| | | KHVAQILDSRMNTKYDENDKLIREVKVITLKSKLVSDFRKDFQFYKVREIN |
| | | NYHHAHDAYLNAVVGTALIKKYPKLESEFVYGDYKVYDVRKMIAKSEQEI |
| | | GKATAKYFFYSNIMNFFKTEITLANGEIRKRPLIETNGETGEIVWDKGRDFA |
| | | TVRKVLSMPQVNIVKKTEVQTGGFSKESILPKRNSDKLIARKKDWDPKKYG |
| | | GFDSPTVAYSVLVVAKVEKGKSKKLKSVKELLGITIMERSSFEKNPIDFLEA |
| | | KGYKEVKKDLIIKLPKYSLFELENGRKRMLASAGELQKGNELALPSKYVNF |
| | | LYLASHYEKLKGSPEDNEQKQLFVEQHKHYLDEIIEQISEFSKRVILADANL |
| | | DKVLSAYNKHRDKPIREQAENIIHLFTLTNLGAPAAFKYFDTTIDRKRYTST |
| | | KEVLDATLIHQSITGLYETRIDLSQLGGD |
| 3 | dCas9 | MDKKYSIGLAIGTNSVGWAVITDEYKVPSKKFKVLGNTDRHSIKKNLIGAL |
| | | LFDSGETAEATRLKRTARRRYTRRKNRICYLQEIFSNEMAKVDDSFFHRLEE |
| | | SFLVEEDKKHERHPIFGNIVDEVAYHEKYPTIYHLRKKLVDSTDKADLRLIY |
| | | LALAHMIKFRGHFLIEGDLNPDNSDVDKLFIQLVQTYNQLFEENPINASGVD |
| | | AKAILSARLSKSRRLENLIAQLPGEKKNGLFGNLIALSLGLTPNFKSNFDLAE |
| | | DAKLQLSKDTYDDDLDNLLAQIGDQYADLFLAAKNLSDAILLSDILRVNTEI |
| | | TKAPLSASMIKRYDEHHQDLTLLKALVRQQLPEKYKEIFFDQSKNGYAGYI |
| | | DGGASQEEFYKFIKPILEKMDGTEELLVKLNREDLLRKQRTFDNGSIPHQIH |
| | | LGELHAILRRQEDFYPFLKDNREKIEKILTFRIPYYVGPLARGNSRFAWMTR |
| | | KSEETITPWNFEEVVDKGASAQSFIERMTNFDKNLPNEKVLPKHSLLYEYFT |
| | | VYNELTKVKYVTEGMRKPAFLSGEQKKAIVDLLFKTNRKVTVKQLKEDYF |
| | | KKIECFDSVEISGVEDRFNASLGTYHDLLKIIKDKDFLDNEENEDILEDIVLTL |
| | | TLFEDREMIEERLKTYAHLFDDKVMKQLKRRRYTGWGRLSRKLINGIRDK |
| | | QSGKTILDFLKSDGFANRNFMQLIHDDSLTFKEDIQKAQVSGQGDSLHEHIA |
| | | NLAGSPAIKKGILQTVKVVDELVKVMGRHKPENIVIEMARENQTTQKGQK |
| | | NSRERMKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSEEVVK |
| | | KMKNYWRQLLNAKLITQRKFDNLTKAERGGLSELDKAGFIKRQLVETRQIT |
| | | KHVAQILDSRMNTKYDENDKLIREVKVITLKSKLVSDFRKDFQFYKVREIN |
| | | NYHHAHDAYLNAVVGTALIKKYPKLESEFVYGDYKVYDVRKMIAKSEQEI |
| | | GKATAKYFFYSNIMNFFKTEITLANGEIRKRPLIETNGETGEIVWDKGRDFA |
| | | TVRKVLSMPQVNIVKKTEVQTGGFSKESILPKRNSDKLIARKKDWDPKKYG |
| | | GFDSPTVAYSVLVVAKVEKGKSKKLKSVKELLGITIMERSSFEKNPIDFLEA |
| | | KGYKEVKKDLIIKLPKYSLFELENGRKRMLASAGELQKGNELALPSKYVNF |
| | | LYLASHYEKLKGSPEDNEQKQLFVEQHKHYLDEIIEQISEFSKRVILADANL |
| | | DKVLSAYNKHRDKPIREQAENIIHLFTLTNLGAPAAFKYFDTTIDRKRYTST |
| | | KEVLDATLIHQSITGLYETRIDLSQLGGD |
| 4 | inactive | MDKKYSIGLAIGTNSVGWAVITDEYKVPSKKFKVLGNTDRHSIKKNLIGAL |
| | VRER | LFDSGETAEATRLKRTARRRYTRRKNRICYLQEIFSNEMAKVDDSFFHRLEE |
| | SpCas9 | SFLVEEDKKHERHPIFGNIVDEVAYHEKYPTIYHLRKKLVDSTDKADLRLIY |
| | | LALAHMIKFRGHFLIEGDLNPDNSDVDKLFIQLVQTYNQLFEENPINASGVD |
| | | AKAILSARLSKSRRLENLIAQLPGEKKNGLFGNLIALSLGLTPNFKSNFDLAE |
| | | DAKLQLSKDTYDDDLDNLLAQIGDQYADLFLAAKNLSDAILLSDILRVNTEI |
| | | TKAPLSASMIKRYDEHHQDLTLLKALVRQQLPEKYKEIFFDQSKNGYAGYI |
| | | DGGASQEEFYKFIKPILEKMDGTEELLVKLNREDLLRKQRTFDNGSIPHQIH |
| | | LGELHAILRRQEDFYPFLKDNREKIEKILTFRIPYYVGPLARGNSRFAWMTR |
| | | KSEETITPWNFEEVVDKGASAQSFIERMTNFDKNLPNEKVLPKHSLLYEYFT |
| | | VYNELTKVKYVTEGMRKPAFLSGEQKKAIVDLLFKTNRKVTVKQLKEDYF |
| | | KKIECFDSVEISGVEDRFNASLGTYHDLLKIIKDKDFLDNEENEDILEDIVLTL |
| | | TLFEDREMIEERLKTYAHLFDDKVMKQLKRRRYTGWGRLSRKLINGIRDK |
| | | QSGKTILDFLKSDGFANRNFMQLIHDDSLTFKEDIQKAQVSGQGDSLHEHIA |
| | | NLAGSPAIKKGILQTVKVVDELVKVMGRHKPENIVIEMARENQTTQKGQK |
| | | NSRERMKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSEEVVK |
| | | KMKNYWRQLLNAKLITQRKFDNLTKAERGGLSELDKAGFIKRQLVETRQIT |
| | | KHVAQILDSRMNTKYDENDKLIREVKVITLKSKLVSDFRKDFQFYKVREIN |
| | | NYHHAHDAYLNAVVGTALIKKYPKLESEFVYGDYKVYDVRKMIAKSEQEI |
| | | GKATAKYFFYSNIMNFFKTEITLANGEIRKRPLIETNGETGEIVWDKGRDFA |
| | | TVRKVLSMPQVNIVKKTEVQTGGFSKESILPKRNSDKLIARKKDWDPKKYG |
| | | GFVSPTVAYSVLVVAKVEKGKSKKLKSVKELLGITIMERSSFEKNPIDFLEA |
| | | KGYKEVKKDLIIKLPKYSLFELENGRKRMLASARELQKGNELALPSKYVNF |
| | | LYLASHYEKLKGSPEDNEQKQLFVEQHKHYLDEIIEQISEFSKRVILADANL |
| | | DKVLSAYNKHRDKPIREQAENIIHLFTLTNLGAPAAFKYFDTTIDRKEYRST |
| | | KEVLDATLIHQSITGLYETRIDLSQLGGD |
| 5 | inactive | MDKKYSIGLAIGTNSVGWAVITDEYKVPSKKFKVLGNTDRHSIKKNLIGAL |
| | EQR | LFDSGETAEATRLKRTARRRYTRRKNRICYLQEIFSNEMAKVDDSFFHRLEE |
| | SpCas9 | SFLVEEDKKHERHPIFGNIVDEVAYHEKYPTIYHLRKKLVDSTDKADLRLIY |
| | | LALAHMIKFRGHFLIEGDLNPDNSDVDKLFIQLVQTYNQLFEENPINASGVD |
| | | AKAILSARLSKSRRLENLIAQLPGEKKNGLFGNLIALSLGLTPNFKSNFDLAE |
| | | DAKLQLSKDTYDDDLDNLLAQIGDQYADLFLAAKNLSDAILLSDILRVNTEI |
| | | TKAPLSASMIKRYDEHHQDLTLLKALVRQQLPEKYKEIFFDQSKNGYAGYI |
| | | DGGASQEEFYKFIKPILEKMDGTEELLVKLNREDLLRKQRTFDNGSIPHQIH |
| | | LGELHAILRRQEDFYPFLKDNREKIEKILTFRIPYYVGPLARGNSRFAWMTR |
| | | KSEETITPWNFEEVVDKGASAQSFIERMTNFDKNLPNEKVLPKHSLLYEYFT |
| | | VYNELTKVKYVTEGMRKPAFLSGEQKKAIVDLLFKTNRKVTVKQLKEDYF |
| | | KKIECFDSVEISGVEDRFNASLGTYHDLLKIIKDKDFLDNEENEDILEDIVLTL |
| | | TLFEDREMIEERLKTYAHLFDDKVMKQLKRRRYTGWGRLSRKLINGIRDK |
| | | QSGKTILDFLKSDGFANRNFMQLIHDDSLTFKEDIQKAQVSGQGDSLHEHIA |
| | | NLAGSPAIKKGILQTVKVVDELVKVMGRHKPENIVIEMARENQTTQKGQK |
| | | NSRERMKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSEEVVK |
| | | KMKNYWRQLLNAKLITQRKFDNLTKAERGGLSELDKAGFIKRQLVETRQIT |
| | | KHVAQILDSRMNTKYDENDKLIREVKVITLKSKLVSDFRKDFQFYKVREIN |
| | | NYHHAHDAYLNAVVGTALIKKYPKLESEFVYGDYKVYDVRKMIAKSEQEI |
| | | GKATAKYFFYSNIMNFFKTEITLANGEIRKRPLIETNGETGEIVWDKGRDFA |
| | | TVRKVLSMPQVNIVKKTEVQTGGFSKESILPKRNSDKLIARKKDWDPKKYG |
| | | GFESPTVAYSVLVVAKVEKGKSKKLKSVKELLGITIMERSSFEKNPIDFLEA |
| | | KGYKEVKKDLIIKLPKYSLFELENGRKRMLASAGELQKGNELALPSKYVNF |
| | | LYLASHYEKLKGSPEDNEQKQLFVEQHKHYLDEIIEQISEFSKRVILADANL |
| | | DKVLSAYNKHRDKPIREQAENIIHLFTLTNLGAPAAFKYFDTTIDRKQYRST |
| | | KEVLDATLIHQSITGLYETRIDLSQLGGD |
| 6 | inactive | MDKKYSIGLAIGTNSVGWAVITDEYKVPSKKFKVLGNTDRHSIKKNLIGAL |
| | VQR | LFDSGETAEATRLKRTARRRYTRRKNRICYLQEIFSNEMAKVDDSFFHRLEE |
| | SpCas9 | SFLVEEDKKHERHPIFGNIVDEVAYHEKYPTIYHLRKKLVDSTDKADLRLIY |
| | | LALAHMIKFRGHFLIEGDLNPDNSDVDKLFIQLVQTYNQLFEENPINASGVD |
| | | AKAILSARLSKSRRLENLIAQLPGEKKNGLFGNLIALSLGLTPNFKSNFDLAE |
| | | DAKLQLSKDTYDDDLDNLLAQIGDQYADLFLAAKNLSDAILLSDILRVNTEI |
| | | TKAPLSASMIKRYDEHHQDLTLLKALVRQQLPEKYKEIFFDQSKNGYAGYI |
| | | DGGASQEEFYKFIKPILEKMDGTEELLVKLNREDLLRKQRTFDNGSIPHQIH |
| | | LGELHAILRRQEDFYPFLKDNREKIEKILTFRIPYYVGPLARGNSRFAWMTR |
| | | KSEETITPWNFEEVVDKGASAQSFIERMTNFDKNLPNEKVLPKHSLLYEYFT |
| | | VYNELTKVKYVTEGMRKPAFLSGEQKKAIVDLLFKTNRKVTVKQLKEDYF |
| | | KKIECFDSVEISGVEDRFNASLGTYHDLLKIIKDKDFLDNEENEDILEDIVLTL |
| | | TLFEDREMIEERLKTYAHLFDDKVMKQLKRRRYTGWGRLSRKLINGIRDK |
| | | QSGKTILDFLKSDGFANRNFMQLIHDDSLTFKEDIQKAQVSGQGDSLHEHIA |
| | | NLAGSPAIKKGILQTVKVVDELVKVMGRHKPENIVIEMARENQTTQKGQK |
| | | NSRERMKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSEEVVK |
| | | KMKNYWRQLLNAKLITQRKFDNLTKAERGGLSELDKAGFIKRQLVETRQIT |
| | | KHVAQILDSRMNTKYDENDKLIREVKVITLKSKLVSDFRKDFQFYKVREIN |
| | | NYHHAHDAYLNAVVGTALIKKYPKLESEFVYGDYKVYDVRKMIAKSEQEI |
| | | GKATAKYFFYSNIMNFFKTEITLANGEIRKRPLIETNGETGEIVWDKGRDFA |
| | | TVRKVLSMPQVNIVKKTEVQTGGFSKESILPKRNSDKLIARKKDWDPKKYG |
| | | GFVSPTVAYSVLVVAKVEKGKSKKLKSVKELLGITIMERSSFEKNPIDFLEA |
| | | KGYKEVKKDLIIKLPKYSLFELENGRKRMLASAGELQKGNELALPSKYVNF |
| | | LYLASHYEKLKGSPEDNEQKQLFVEQHKHYLDEIIEQISEFSKRVILADANL |
| | | DKVLSAYNKHRDKPIREQAENIIHLFTLTNLGAPAAFKYFDTTIDRKQYRST |
| | | KEVLDATLIHQSITGLYETRIDLSQLGGD |
| 7 | inactive | MDKKYSIGLAIGTNSVGWAVITDEYKVPSKKFKVLGNTDRHSIKKNLIGAL |
| | SPG | LFDSGETAEATRLKRTARRRYTRRKNRICYLQEIFSNEMAKVDDSFFHRLEE |
| | SpCas9 | SFLVEEDKKHERHPIFGNIVDEVAYHEKYPTIYHLRKKLVDSTDKADLRLIY |
| | | LALAHMIKFRGHFLIEGDLNPDNSDVDKLFIQLVQTYNQLFEENPINASGVD |
| | | AKAILSARLSKSRRLENLIAQLPGEKKNGLFGNLIALSLGLTPNFKSNFDLAE |
| | | DAKLQLSKDTYDDDLDNLLAQIGDQYADLFLAAKNLSDAILLSDILRVNTEI |
| | | TKAPLSASMIKRYDEHHQDLTLLKALVRQQLPEKYKEIFFDQSKNGYAGYI |
| | | DGGASQEEFYKFIKPILEKMDGTEELLVKLNREDLLRKQRTFDNGSIPHQIH |
| | | LGELHAILRRQEDFYPFLKDNREKIEKILTFRIPYYVGPLARGNSRFAWMTR |
| | | KSEETITPWNFEEVVDKGASAQSFIERMTNFDKNLPNEKVLPKHSLLYEYFT |
| | | VYNELTKVKYVTEGMRKPAFLSGEQKKAIVDLLFKTNRKVTVKQLKEDYF |
| | | KKIECFDSVEISGVEDRFNASLGTYHDLLKIIKDKDFLDNEENEDILEDIVLTL |
| | | TLFEDREMIEERLKTYAHLFDDKVMKQLKRRRYTGWGRLSRKLINGIRDK |
| | | QSGKTILDFLKSDGFANRNFMQLIHDDSLTFKEDIQKAQVSGQGDSLHEHIA |
| | | NLAGSPAIKKGILQTVKVVDELVKVMGRHKPENIVIEMARENQTTQKGQK |
| | | NSRERMKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSEEVVK |
| | | KMKNYWRQLLNAKLITQRKFDNLTKAERGGLSELDKAGFIKRQLVETRQIT |
| | | KHVAQILDSRMNTKYDENDKLIREVKVITLKSKLVSDFRKDFQFYKVREIN |
| | | NYHHAHDAYLNAVVGTALIKKYPKLESEFVYGDYKVYDVRKMIAKSEQEI |
| | | GKATAKYFFYSNIMNFFKTEITLANGEIRKRPLIETNGETGEIVWDKGRDFA |
| | | TVRKVLSMPQVNIVKKTEVQTGGFSKESILPKRNSDKLIARKKDWDPKKYG |
| | | GFLWPTVAYSVLVVAKVEKGKSKKLKSVKELLGITIMERSSFEKNPIDFLEA |
| | | KGYKEVKKDLIIKLPKYSLFELENGRKRMLASAKQLQKGNELALPSKYVNF |
| | | LYLASHYEKLKGSPEDNEQKQLFVEQHKHYLDEIIEQISEFSKRVILADANL |
| | | DKVLSAYNKHRDKPIREQAENIIHLFTLTNLGAPAAFKYFDTTIDRKQYRST |
| | | KEVLDATLIHQSITGLYETRIDLSQLGGD |
| 8 | inactive | MDKKYSIGLAIGTNSVGWAVITDEYKVPSKKFKVLGNTDRHSIKKNLIGAL |
| | SpRY Cas9 | LFDSGETAERTRLKRTARRRYTRRKNRICYLQEIFSNEMAKVDDSFFHRLEE |
| | | SFLVEEDKKHERHPIFGNIVDEVAYHEKYPTIYHLRKKLVDSTDKADLRLIY |
| | | LALAHMIKFRGHFLIEGDLNPDNSDVDKLFIQLVQTYNQLFEENPINASGVD |
| | | AKAILSARLSKSRRLENLIAQLPGEKKNGLFGNLIALSLGLTPNFKSNFDLAE |
| | | DAKLQLSKDTYDDDLDNLLAQIGDQYADLFLAAKNLSDAILLSDILRVNTEI |
| | | TKAPLSASMIKRYDEHHQDLTLLKALVRQQLPEKYKEIFFDQSKNGYAGYI |
| | | DGGASQEEFYKFIKPILEKMDGTEELLVKLNREDLLRKQRTFDNGSIPHQIH |
| | | LGELHAILRRQEDFYPFLKDNREKIEKILTFRIPYYVGPLARGNSRFAWMTR |
| | | KSEETITPWNFEEVVDKGASAQSFIERMTNFDKNLPNEKVLPKHSLLYEYFT |
| | | VYNELTKVKYVTEGMRKPAFLSGEQKKAIVDLLFKTNRKVTVKQLKEDYF |
| | | KKIECFDSVEISGVEDRFNASLGTYHDLLKIIKDKDFLDNEENEDILEDIVLTL |
| | | TLFEDREMIEERLKTYAHLFDDKVMKQLKRRRYTGWGRLSRKLINGIRDK |
| | | QSGKTILDFLKSDGFANRNFMQLIHDDSLTFKEDIQKAQVSGQGDSLHEHIA |
| | | NLAGSPAIKKGILQTVKVVDELVKVMGRHKPENIVIEMARENQTTQKGQK |
| | | NSRERMKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSEEVVK |
| | | KMKNYWRQLLNAKLITQRKFDNLTKAERGGLSELDKAGFIKRQLVETRQIT |
| | | KHVAQILDSRMNTKYDENDKLIREVKVITLKSKLVSDFRKDFQFYKVREIN |
| | | NYHHAHDAYLNAVVGTALIKKYPKLESEFVYGDYKVYDVRKMIAKSEQEI |
| | | GKATAKYFFYSNIMNFFKTEITLANGEIRKRPLIETNGETGEIVWDKGRDFA |
| | | TVRKVLSMPQVNIVKKTEVQTGGFSKESIRPKRNSDKLIARKKDWDPKKYG |
| | | GFLWPTVAYSVLVVAKVEKGKSKKLKSVKELLGITIMERSSFEKNPIDFLEA |
| | | KGYKEVKKDLIIKLPKYSLFELENGRKRMLASAKQLQKGNELALPSKYVNF |
| | | LYLASHYEKLKGSPEDNEQKQLFVEQHKHYLDEIIEQISEFSKRVILADANL |
| | | DKVLSAYNKHRDKPIREQAENIIHLFTLTRLGAPRAFKYFDTTIDPKQYRST |
| | | KEVLDATLIHQSITGLYETRIDLSQLGGD |
| 9 | SaCas9 | MKRNYILGLDIGITSVGYGIIDYETRDVIDAGVRLFKEANVENNEGRRSKRG |
| | | ARRLKRRRRHRIQRVKKLLFDYNLLTDHSELSGINPYEARVKGLSQKLSEEE |
| | | FSAALLHLAKRRGVHNVNEVEEDTGNELSTKEQISRNSKALEEKYVAELQL |
| | | ERLKKDGEVRGSINRFKTSDYVKEAKQLLKVQKAYHQLDQSFIDTYIDLLE |
| | | TRRTYYEGPGEGSPFGWKDIKEWYEMLMGHCTYFPEELRSVKYAYNADLY |
| | | NALNDLNNLVITRDENEKLEYYEKFQIIENVFKQKKKPTLKQIAKEILVNEE |
| | | DIKGYRVTSTGKPEFTNLKVYHDIKDITARKEIIENAELLDQIAKILTIYQSSE |
| | | DIQEELTNLNSELTQEEIEQISNLKGYTGTHNLSLKAINLILDELWHTNDNQI |
| | | AIFNRLKLVPKKVDLSQQKEIPTTLVDDFILSPVVKRSFIQSIKVINAIIKKYG |
| | | LPNDIIIELAREKNSKDAQKMINEMQKRNRQTNERIEEIIRTTGKENAKYLIE |
| | | KIKLHDMQEGKCLYSLEAIPLEDLLNNPFNYEVDHIIPRSVSFDNSFNNKVL |
| | | VKQEEASKKGNRTPFQYLSSSDSKISYETFKKHILNLAKGKGRISKTKKEYL |
| | | LEERDINRFSVQKDFINRNLVDTRYATRGLMNLLRSYFRVNNLDVKVKSIN |
| | | GGFTSFLRRKWKFKKERNKGYKHHAEDALIIANADFIFKEWKKLDKAKKV |
| | | MENQMFEEKQAESMPEIETEQEYKEIFITPHQIKHIKDFKDYKYSHRVDKKP |
| | | NRELINDTLYSTRKDDKGNTLIVNNLNGLYDKDNDKLKKLINKSPEKLLMY |
| | | HHDPQTYQKLKLIMEQYGDEKNPLYKYYEETGNYLTKYSKKDNGPVIKKI |
| | | KYYGNKLNAHLDITDDYPNSRNKVVKLSLKPYRFDVYLDNGVYKFVTVK |
| | | NLDVIKKENYYEVNSKAYEEAKKLKKISNQAEFIASFYNNDLIKINGELYRV |
| | | IGVNNDLLNRIEVNMIDITYREYLENMNDKRPPRIIKTIASKTQSIKKYSTDIL |
| | | GNLYEVKSKKHPQIIKKG |
| 10 | inactive | MKRNYILGLAIGITSVGYGIIDYETRDVIDAGVRLFKEANVENNEGRRSKRG |
| | KKH | ARRLKRRRRHRIQRVKKLLFDYNLLTDHSELSGINPYEARVKGLSQKLSEEE |
| | dSaCas9 | FSAALLHLAKRRGVHNVNEVEEDTGNELSTKEQISRNSKALEEKYVAELQL |
| | | ERLKKDGEVRGSINRFKTSDYVKEAKQLLKVQKAYHQLDQSFIDTYIDLLE |
| | | TRRTYYEGPGEGSPFGWKDIKEWYEMLMGHCTYFPEELRSVKYAYNADLY |
| | | NALNDLNNLVITRDENEKLEYYEKFQIIENVFKQKKKPTLKQIAKEILVNEE |
| | | DIKGYRVTSTGKPEFTNLKVYHDIKDITARKEIIENAELLDQIAKILTIYQSSE |
| | | DIQEELTNLNSELTQEEIEQISNLKGYTGTHNLSLKAINLILDELWHTNDNQI |
| | | AIFNRLKLVPKKVDLSQQKEIPTTLVDDFILSPVVKRSFIQSIKVINAIIKKYG |
| | | LPNDIIIELAREKNSKDAQKMINEMQKRNRQTNERIEEIIRTTGKENAKYLIE |
| | | KIKLHDMQEGKCLYSLEAIPLEDLLNNPFNYEVDHIIPRSVSFDNSFNNKVL |
| | | VKQEEASKKGNRTPFQYLSSSDSKISYETFKKHILNLAKGKGRISKTKKEYL |
| | | LEERDINRFSVQKDFINRNLVDTRYATRGLMNLLRSYFRVNNLDVKVKSIN |
| | | GGFTSFLRRKWKFKKERNKGYKHHAEDALIIANADFIFKEWKKLDKAKKV |
| | | MENQMFEEKQAESMPEIETEQEYKEIFITPHQIKHIKDFKDYKYSHRVDKKP |
| | | NRKLINDTLYSTRKDDKGNTLIVNNLNGLYDKDNDKLKKLINKSPEKLLMY |
| | | HHDPQTYQKLKLIMEQYGDEKNPLYKYYEETGNYLTKYSKKDNGPVIKKI |
| | | KYYGNKLNAHLDITDDYPNSRNKVVKLSLKPYRFDVYLDNGVYKFVTVK |
| | | NLDVIKKENYYEVNSKCYEEAKKLKKISNQAEFIASFYKNDLIKINGELYRV |
| | | IGVNNDLLNRIEVNMIDITYREYLENMNDKRPPHIIKTIASKTQSIKKYSTDIL |
| | | GNLYEVKSKKHPQIIKKG |
| 11 | dNmeCas9 | MAAFKPNSINYILGLAIGIASVGWAMVEIDEEENPIRLIDLGVRVFERAEVPK |
| | | TGDSLAMARRLARSVRRLTRRRAHRLLRTRRLLKREGVLQAANFDENGLI |
| | | KSLPNTPWQLRAAALDRKLTPLEWSAVLLHLIKHRGYLSQRKNEGETADK |
| | | ELGALLKGVAGNAHALQTGDFRTPAELALNKFEKESGHIRNQRSDYSHTFS |
| | | RKDLQAELILLFEKQKEFGNPHVSGGLKEGIETLLMTQRPALSGDAVQKML |
| | | GHCTFEPAEPKAAKNTYTAERFIWLTKLNNLRILEQGSERPLTDTERATLMD |
| | | EPYRKSKLTYAQARKLLGLEDTAFFKGLRYGKDNAEASTLMEMKAYHAIS |
| | | RALEKEGLKDKKSPLNLSPELQDEIGTAFSLFKTDEDITGRLKDRIQPEILEA |
| | | LLKHISFDKFVQISLKALRRIVPLMEQGKRYDEACAEIYGDHYGKKNTEEKI |
| | | YLPPIPADEIRNPVVLRALSQARKVINGVVRRYGSPARIHIETAREVGKSFKD |
| | | RKEIEKRQEENRKDREKAAAKFREYFPNFVGEPKSKDILKLRLYEQQHGKC |
| | | LYSGKEINLGRLNEKGYVEIDAALPFSRTWDDSFNNKVLVLGSENQNKGNQ |
| | | TPYEYFNGKDNSREWQEFKARVETSRFPRSKKQRILLQKFDEDGFKERNLN |
| | | DTRYVNRFLCQFVADRMRLTGKGKKRVFASNGQITNLLRGFWGLRKVRAE |
| | | NDRHHALDAVVVACSTVAMQQKITRFVRYKEMNAFDGKTIDKETGEVLH |
| | | QKTHFPQPWEFFAQEVMIRVFGKPDGKPEFEEADTLEKLRTLLAEKLSSRPE |
| | | AVHEYVTPLFVSRAPNRKMSGQGHMETVKSAKRLDEGVSVLRVPLTQLKL |
| | | KDLEKMVNREREPKLYEALKARLEAHKDDPAKAFAEPFYKYDKAGNRTQ |
| | | QVKAVRVEQVQKTGVWVRNHNGIADNATMVRVDVFEKGDKYYLVPIYS |
| | | WQVAKGILPDRAVVQGKDEEDWQLIDDSFNFKFSLHPNDLVEVITKKARM |
| | | FGYFASCHRGTGNINIRIHDLDHKIGKNGILEGIGVKTALSFQKYQIDELGKE |
| | | IRPCRLKKRPPVR |
| 12 | dCjCas9 | MARILAFAIGISSIGWAFSENDELKDCGVRIFTKVENPKTGESLALPRRLARS |
| | | ARKRLARRKARLNHLKHLIANEFKLNYEDYQSFDESLAKAYKGSLISPYEL |
| | | RFRALNELLSKQDFARVILHIAKRRGYDDIKNSDDKEKGAILKAIKQNEEKL |
| | | ANYQSVGEYLYKEYFQKFKENSKEFTNVRNKKESYERCIAQSFLKDELKLIF |
| | | KKQREFGFSFSKKFEEEVLSVAFYKRALKDFSHLVGNCSFFTDEKRAPKNSP |
| | | LAFMFVALTRIINLLNNLKNTEGILYTKDDLNALLNEVLKNGTLTYKQTKK |
| | | LLGLSDDYEFKGEKGTYFIEFKKYKEFIKALGEHNLSQDDLNEIAKDITLIKD |
| | | EIKLKKALAKYDLNQNQIDSLSKLEFKDHLNISFKALKLVTPLMLEGKKYD |
| | | EACNELNLKVAINEDKKDFLPAFNETYYKDEVTNPVVLRAIKEYRKVLNAL |
| | | LKKYGKVHKINIELAREVGKNHSQRAKIEKEQNENYKAKKDAELECEKLG |
| | | LKINSKNILKLRLFKEQKEFCAYSGEKIKISDLQDEKMLEIDAIYPYSRSFDDS |
| | | YMNKVLVFTKQNQEKLNQTPFEAFGNDSAKWQKIEVLAKNLPTKKQKRIL |
| | | DKNYKDKEQKNFKDRNLNDTRYIARLVLNYTKDYLDFLPLSDDENTKLND |
| | | TQKGSKVHVEAKSGMLTSALRHTWGFSAKDRNNHLHHAIDAVIIAYANNSI |
| | | VKAFSDFKKEQESNSAELYAKKISELDYKNKRKFFEPFSGFRQKVLDKIDEI |
| | | FVSKPERKKPSGALHEETFRKEEEFYQSYGGKEGVLKALELGKIRKVNGKI |
| | | VKNGDMFRVDIFKHKKTNKFYAVPIYTMDFALKVLPNKAVARSKKGEIKD |
| | | WILMDENYEFCFSLYKDSLILIQTKDMQEPEFVYYNAFTSSTVSLIVSKHDN |
| | | KFETLSKNQKILFKNANEKEVIAKSIGIQNLKVFEKYIVSALGEVTKAEFRQR |
| | | EDFKK |
| 13 | dSt1Cas9 | MGSDLVLGLAIGIGSVGVGILNKVTGEIIHKNSRIFPAAQAENNLVRRTNRQ |
| | | GRRLARRKKHRRVRLNRLFEESGLITDFTKISINLNPYQLRVKGLTDELSNE |
| | | ELFIALKNMVKHRGISYLDDASDDGNSSVGDYAQIVKENSKQLETKTPGQI |
| | | QLERYQTYGQLRGDFTVEKDGKKHRLINVFPTSAYRSEALRILQTQQEFNP |
| | | QITDEFINRYLEILTGKRKYYHGPGNEKSRTDYGRYRTSGETLDNIFGILIGK |
| | | CTFYPDEFRAAKASYTAQEFNLLNDLNNLTVPTETKKLSKEQKNQIINYVK |
| | | NEKAMGPAKLFKYIAKLLSCDVADIKGYRIDKSGKAEIHTFEAYRKMKTLE |
| | | TLDIEQMDRETLDKLAYVLTLNTEREGIQEALEHEFADGSFSQKQVDELVQ |
| | | FRKANSSIFGKGWHNFSVKLMMELIPELYETSEEQMTILTRLGKQKTTSSSN |
| | | KTKYIDEKLLTEEIYNPVVAKSVRQAIKIVNAAIKEYGDFDNIVIEMARETNE |
| | | DDEKKAIQKIQKANKDEKDAAMLKAANQYNGKAELPHSVFHGHKQLATK |
| | | IRLWHQQGERCLYTGKTISIHDLINNSNQFEVDAILPLSITFDDSLANKVLVY |
| | | ATANQEKGQRTPYQALDSMDDAWSFRELKAFVRESKTLSNKKKEYLLTEE |
| | | DISKFDVRKKFIERNLVDTRYASRVVLNALQEHFRAHKIDTKVSVVRGQFT |
| | | SQLRRHWGIEKTRDTYHHHAVDALIIAASSQLNLWKKQKNTLVSYSEDQLL |
| | | DIETGELISDDEYKESVFKAPYQHFVDTLKSKEFEDSILFSYQVDSKFNRKIS |
| | | DATIYATRQAKVGKDKADETYVLGKIKDIYTQDGYDAFMKIYKKDKSKFL |
| | | MYRHDPQTFEKVIEPILENYPNKQINEKGKEVPCNPFLKYKEEHGYIRKYSK |
| | | KGNGPEIKSLKYYDSKLGNHIDITPKDSNNKVVLQSVSPWRADVYFNKTTG |
| | | KYEILGLKYADLQFEKGTGTYKISQEKYNDIKKKEGVDSDSEFKFTLYKND |
| | | LLLVKDTETKEQQLFRFLSRTMPKQKHYVELKPYDKQKFEGGEALIKVLGN |
| | | VANSGQCKKGLGKSNISIYKVRTDVLGNQHIIKNEGDKPKLDF |
| 14 | dSt3Cas9 | MTKPYSIGLAIGTNSVGWAVITDNYKVPSKKMKVLGNTSKKYIKKNLLGV |
| | | LLFDSGITAEGRRLKRTARRRYTRRRNRILYLQEIFSTEMATLDDAFFQRLD |
| | | DSFLVPDDKRDSKYPIFGNLVEEKVYHDEFPTIYHLRKYLADSTKKADLRL |
| | | VYLALAHMIKYRGHFLIEGEFNSKNNDIQKNFQDFLDTYNAIFESDLSLENS |
| | | KQLEEIVKDKISKLEKKDRILKLFPGEKNSGIFSEFLKLIVGNQADFRKCFNL |
| | | DEKASLHFSKESYDEDLETLLGYIGDDYSDVFLKAKKLYDAILLSGFLTVTD |
| | | NETEAPLSSAMIKRYNEHKEDLALLKEYIRNISLKTYNEVFKDDTKNGYAG |
| | | YIDGKTNQEDFYVYLKNLLAEFEGADYFLEKIDREDFLRKQRTFDNGSIPYQ |
| | | IHLQEMRAILDKQAKFYPFLAKNKERIEKILTFRIPYYVGPLARGNSDFAWSI |
| | | RKRNEKITPWNFEDVIDKESSAEAFINRMTSFDLYLPEEKVLPKHSLLYETF |
| | | NVYNELTKVRFIAESMRDYQFLDSKQKKDIVRLYFKDKRKVTDKDIIEYLH |
| | | AIYGYDGIELKGIEKQFNSSLSTYHDLLNIINDKEFLDDSSNEAIIEEIIHTLTIF |
| | | EDREMIKQRLSKFENIFDKSVLKKLSRRHYTGWGKLSAKLINGIRDEKSGNT |
| | | ILDYLIDDGISNRNFMQLIHDDALSFKKKIQKAQIIGDEDKGNIKEVVKSLPG |
| | | SPAIKKGILQSIKIVDELVKVMGGRKPESIVVEMARENQYTNQGKSNSQQRL |
| | | KRLEKSLKELGSKILKENIPAKLSKIDNNALQNDRLYLYYLQNGKDMYTGD |
| | | DLDIDRLSNYDIDHIIPQAFLKDNSIDNKVLVSSASARGKSDDFPSLEVVKKR |
| | | KTFWYQLLKSKLISQRKFDNLTKAERGGLLPEDKAGFIQRQLVETRQITKH |
| | | VARLLDEKFNNKKDENNRAVRTVKIITLKSTLVSQFRKDFELYKVREINDFH |
| | | HAHDAYLNAVIASALLKKYPKLEPEFVYGDYPKYNSFRERKSATEKVYFYS |
| | | NIMNIFKKSISLADGRVIERPLIEVNEETGESVWNKESDLATVRRVLSYPQV |
| | | NVVKKVEEQNHGLDRGKPKGLFNANLSSKPKPNSNENLVGAKEYLDPKKY |
| | | GGYAGISNSFAVLVKGTIEKGAKKKITNVLEFQGISILDRINYRKDKLNFLLE |
| | | KGYKDIELIIELPKYSLFELSDGSRRMLASILSTNNKRGEIHKGNQIFLSQKFV |
| | | KLLYHAKRISNTINENHRKYVENHKKEFEELFYYILEFNENYVGAKKNGKL |
| | | LNSAFQSWQNHSIDELCSSFIGPTGSERKGLFELTSRGSAADFEFLGVKIPRY |
| | | RDYTPSSLLKDATLIHQSVTGLYETRIDLAKLGEG |
| 15 | F. novicida | MSIYQEFVNKYSLSKTLRFELIPQGKTLENIKARGLILDDEKRAKDYKKAKQ |
| | WT Cpf1 | IIDKYHQFFIEEILSSVCISEDLLQNYSDVYFKLKKSDDDNLQKDFKSAKDTI |
| | | KKQISEYIKDSEKFKNLFNQNLIDAKKGQESDLILWLKQSKDNGIELFKANS |
| | | DITDIDEALEIIKSFKGWTTYFKGFHENRKNVYSSNDIPTSIIYRIVDDNLPKF |
| | | LENKAKYESLKDKAPEAINYEQIKKDLAEELTFDIDYKTSEVNQRVFSLDEV |
| | | FEIANFNNYLNQSGITKFNTIIGGKFVNGENTKRKGINEYINLYSQQINDKTL |
| | | KKYKMSVLFKQILSDTESKSFVIDKLEDDSDVVTTMQSFYEQIAAFKTVEEK |
| | | SIKETLSLLFDDLKAQKLDLSKIYFKNDKSLTDLSQQVFDDYSVIGTAVLEYI |
| | | TQQIAPKNLDNPSKKEQELIAKKTEKAKYLSLETIKLALEEFNKHRDIDKQC |
| | | RFEEILANFAAIPMIFDEIAQNKDNLAQISIKYQNQGKKDLLQASAEDDVKAI |
| | | KDLLDQTNNLLHKLKIFHISQSEDKANILDKDEHFYLVFEECYFELANIVPL |
| | | YNKIRNYITQKPYSDEKFKLNFENSTLANGWDKNKEPDNTAILFIKDDKYY |
| | | LGVMNKKNNKIFDDKAIKENKGEGYKKIVYKLLPGANKMLPKVFFSAKSIK |
| | | FYNPSEDILRIRNHSTHTKNGSPQKGYEKFEFNIEDCRKFIDFYKQSISKHPE |
| | | WKDFGFRFSDTQRYNSIDEFYREVENQGYKLTFENISESYIDSVVNQGKLYL |
| | | FQIYNKDFSAYSKGRPNLHTLYWKALFDERNLQDVVYKLNGEAELFYRKQ |
| | | SIPKKITHPAKEAIANKNKDNPKKESVFEYDLIKDKRFTEDKFFFHCPITINFK |
| | | SSGANKFNDEINLLLKEKANDVHILSIDRGERHLAYYTLVDGKGNIIKQDTF |
| | | NIIGNDRMKTNYHDKLAAIEKDRDSARKDWKKINNIKEMKEGYLSQVVHEI |
| | | AKLVIEYNAIVVFEDLNFGFKRGRFKVEKQVYQKLEKMLIEKLNYLVFKDN |
| | | EFDKTGGVLRAYQLTAPFETFKKMGKQTGIIYYVPAGFTSKICPVTGFVNQL |
| | | YPKYESVSKSQEFFSKFDKICYNLDKGYFEFSFDYKNFGDKAAKGKWTIAS |
| | | FGSRLINFRNSDKNHNWDTREVYPTKELEKLLKDYSIEYGHGECIKAAICGE |
| | | SDKKFFAKLTSVLNTILQMRNSKTGTELDYLISPVADVNGNFFDSRQAPKN |
| | | MPQDADANGAYHIGLKGLMLLGRIKNNQEGKKLNLVIKNEEYFEFVQNRN |
| | | N |
| 16 | inactive | MSIYQEFVNKYSLSKTLRFELIPQGKTLENIKARGLILDDEKRAKDYKKAKQ |
| | FnCpf1 | IIDKYHQFFIEEILSSVCISEDLLQNYSDVYFKLKKSDDDNLQKDFKSAKDTI |
| | | KKQISEYIKDSEKFKNLFNQNLIDAKKGQESDLILWLKQSKDNGIELFKANS |
| | | DITDIDEALEIIKSFKGWTTYFKGFHENRKNVYSSNDIPTSIIYRIVDDNLPKF |
| | | LENKAKYESLKDKAPEAINYEQIKKDLAEELTFDIDYKTSEVNQRVFSLDEV |
| | | FEIANFNNYLNQSGITKFNTIIGGKFVNGENTKRKGINEYINLYSQQINDKTL |
| | | KKYKMSVLFKQILSDTESKSFVIDKLEDDSDVVTTMQSFYEQIAAFKTVEEK |
| | | SIKETLSLLFDDLKAQKLDLSKIYFKNDKSLTDLSQQVEDDYSVIGTAVLEYI |
| | | TQQIAPKNLDNPSKKEQELIAKKTEKAKYLSLETIKLALEEFNKHRDIDKQC |
| | | RFEEILANFAAIPMIFDEIAQNKDNLAQISIKYQNQGKKDLLQASAEDDVKAI |
| | | KDLLDQTNNLLHKLKIFHISQSEDKANILDKDEHFYLVFEECYFELANIVPL |
| | | YNKIRNYITQKPYSDEKFKLNFENSTLANGWDKNKEPDNTAILFIKDDKYY |
| | | LGVMNKKNNKIFDDKAIKENKGEGYKKIVYKLLPGANKMLPKVFFSAKSIK |
| | | FYNPSEDILRIRNHSTHTKNGSPQKGYEKFEFNIEDCRKFIDFYKQSISKHPE |
| | | WKDFGFRFSDTQRYNSIDEFYREVENQGYKLTFENISESYIDSVVNQGKLYL |
| | | FQIYNKDFSAYSKGRPNLHTLYWKALFDERNLQDVVYKLNGEAELFYRKQ |
| | | SIPKKITHPAKEAIANKNKDNPKKESVFEYDLIKDKRFTEDKFFFHCPITINFK |
| | | SSGANKFNDEINLLLKEKANDVHILSIARGERHLAYYTLVDGKGNIIKQDTF |
| | | NIIGNDRMKTNYHDKLAAIEKDRDSARKDWKKINNIKEMKEGYLSQVVHEI |
| | | AKLVIEYNAIVVFEDLNFGFKRGRFKVEKQVYQKLEKMLIEKLNYLVFKDN |
| | | EFDKTGGVLRAYQLTAPFETFKKMGKQTGIIYYVPAGFTSKICPVTGFVNQL |
| | | YPKYESVSKSQEFFSKFDKICYNLDKGYFEFSFDYKNFGDKAAKGKWTIAS |
| | | FGSRLINFRNSDKNHNWDTREVYPTKELEKLLKDYSIEYGHGECIKAAICGE |
| | | SDKKFFAKLTSVLNTILQMRNSKTGTELDYLISPVADVNGNFFDSRQAPKN |
| | | MPQDADANGAYHIGLKGLMLLGRIKNNQEGKKLNLVIKNEEYFEFVQNRN |
| | | N |
| 17 | inactive | MSKLEKFTNCYSLSKTLRFKAIPVGKTQENIDNKRLLVEDEKRAEDYKGVK |
| | dLbCpf1 | KLLDRYYLSFINDVLHSIKLKNLNNYISLFRKKTRTEKENKELENLEINLRKE |
| | | IAKAFKGNEGYKSLFKKDIIETILPEFLDDKDEIALVNSFNGFTTAFTGFFDN |
| | | RENMFSEEAKSTSIAFRCINENLTRYISNMDIFEKVDAIFDKHEVQEIKEKILN |
| | | SDYDVEDFFEGEFFNFVLTQEGIDVYNAIIGGFVTESGEKIKGLNEYINLYNQ |
| | | KTKQKLPKFKPLYKQVLSDRESLSFYGEGYTSDEEVLEVFRNTLNKNSEIFS |
| | | SIKKLEKLFKNFDEYSSAGIFVKNGPAISTISKDIFGEWNVIRDKWNAEYDDI |
| | | HLKKKAVVTEKYEDDRRKSFKKIGSFSLEQLQEYADADLSVVEKLKEIIIQK |
| | | VDEIYKVYGSSEKLFDADFVLEKSLKKNDAVVAIMKDLLDSVKSFENYIKA |
| | | FFGEGKETNRDESFYGDFVLAYDILLKVDHIYDAIRNYVTQKPYSKDKFKL |
| | | YFQNPQFMGGWDKDKETDYRATILRYGSKYYLAIMDKKYAKCLQKIDKD |
| | | DVNGNYEKINYKLLPGPNKMLPKVFFSKKWMAYYNPSEDIQKIYKNGTFK |
| | | KGDMFNLNDCHKLIDFFKDSISRYPKWSNAYDFNFSETEKYKDIAGFYREV |
| | | EEQGYKVSFESASKKEVDKLVEEGKLYMFQIYNKDFSDKSHGTPNLHTMY |
| | | FKLLFDENNHGQIRLSGGAELFMRRASLKKEELVVHPANSPIANKNPDNPK |
| | | KTTTLSYDVYKDKRFSEDQYELHIPIAINKCPKNIFKINTEVRVLLKHDDNPY |
| | | VIGIARGERNLLYIVVVDGKGNIVEQYSLNEIINNFNGIRIKTDYHSLLDKKE |
| | | KERFEARQNWTSIENIKELKAGYISQVVHKICELVEKYDAVIALEDLNSGFK |
| | | NSRVKVEKQVYQKFEKMLIDKLNYMVDKKSNPCATGGALKGYQITNKFES |
| | | FKSMSTQNGFIFYIPAWLTSKIDPSTGFVNLLKTKYTSIADSKKFISSFDRIMY |
| | | VPEEDLFEFALDYKNFSRTDADYIKKWKLYSYGNRIRIFRNPKKNNVFDWE |
| | | EVCLTSAYKELFNKYGINYQQGDIRALLCEQSDKAFYSSFMALMSLMLQM |
| | | RNSITGRTDVDFLISPVKNSDGIFYDSRNYEAQENAILPKNADANGAYNIAR |
| | | KVLWAIGQFKKAEDEKLDKVKIAISNKEWLEYAQTSVKH |
| 18 | inactive | MTQFEGFTNLYQVSKTLRFELIPQGKTLKHIQEQGFIEEDKARNDHYKELKP |
| | AsCpf1 | IIDRIYKTYADQCLQLVQLDWENLSAAIDSYRKEKTEETRNALIEEQATYRN |
| | | AIHDYFIGRTDNLTDAINKRHAEIYKGLFKAELFNGKVLKQLGTVTTTEHEN |
| | | ALLRSFDKFTTYFSGFYENRKNVFSAEDISTAIPHRIVQDNFPKFKENCHIFT |
| | | RLITAVPSLREHFENVKKAIGIFVSTSIEEVFSFPFYNQLLTQTQIDLYNQLLG |
| | | GISREAGTEKIKGLNEVLNLAIQKNDETAHIIASLPHRFIPLFKQILSDRNTLSF |
| | | ILEEFKSDEEVIQSFCKYKTLLRNENVLETAEALFNELNSIDLTHIFISHKKLE |
| | | TISSALCDHWDTLRNALYERRISELTGKITKSAKEKVQRSLKHEDINLQEIIS |
| | | AAGKELSEAFKQKTSEILSHAHAALDQPLPTTLKKQEEKEILKSQLDSLLGL |
| | | YHLLDWFAVDESNEVDPEFSARLTGIKLEMEPSLSFYNKARNYATKKPYSV |
| | | EKFKLNFQMPTLASGWDVNKEKNNGAILFVKNGLYYLGIMPKQKGRYKA |
| | | LSFEPTEKTSEGFDKMYYDYFPDAAKMIPKCSTQLKAVTAHFQTHTTPILLS |
| | | NNFIEPLEITKEIYDLNNPEKEPKKFQTAYAKKTGDQKGYREALCKWIDFTR |
| | | DFLSKYTKTTSIDLSSLRPSSQYKDLGEYYAELNPLLYHISFQRIAEKEIMDA |
| | | VETGKLYLFQIYNKDFAKGHHGKPNLHTLYWTGLFSPENLAKTSIKLNGQA |
| | | ELFYRPKSRMKRMAHRLGEKMLNKKLKDQKTPIPDTLYQELYDYVNHRLS |
| | | HDLSDEARALLPNVITKEVSHEIIKDRRFTSDKFFFHVPITLNYQAANSPSKF |
| | | NQRVNAYLKEHPETPIIGIARGERNLIYITVIDSTGKILEQRSLNTIQQFDYQK |
| | | KLDNREKERVAARQAWSVVGTIKDLKQGYLSQVIHEIVDLMIHYQAVVVL |
| | | ENLNFGFKSKRTGIAEKAVYQQFEKMLIDKLNCLVLKDYPAEKVGGVLNP |
| | | YQLTDQFTSFAKMGTQSGFLFYVPAPYTSKIDPLTGFVDPFVWKTIKNHESR |
| | | KHFLEGFDFLHYDVKTGDFILHFKMNRNLSFQRGLPGFMPAWDIVFEKNET |
| | | QFDAKGTPFIAGKRIVPVIENHRFTGRYRDLYPANELIALLEEKGIVFRDGSN |
| | | ILPKLLENDDSHAIDTMVALIRSVLQMRNSNAATGEDYINSPVRDLNGVCF |
| | | DSRFQNPEWPMDADANGAYHIALKGQLLLNHLKESKDLKLQNGISNQDWL |
| | | AYIQELRN |
| 19 | inactive | MTQFEGFTNLYQVSKTLRFELIPQGKTLKHIQEQGFIEEDKARNDHYKELKP |
| | enAsCpf1 | IIDRIYKTYADQCLQLVQLDWENLSAAIDSYRKEKTEETRNALIEEQATYRN |
| | | AIHDYFIGRTDNLTDAINKRHAEIYKGLFKAELFNGKVLKQLGTVTTTEHEN |
| | | ALLRSFDKFTTYFSGFYRNRKNVFSAEDISTAIPHRIVQDNFPKFKENCHIFT |
| | | RLITAVPSLREHFENVKKAIGIFVSTSIEEVFSFPFYNQLLTQTQIDLYNQLLG |
| | | GISREAGTEKIKGLNEVLNLAIQKNDETAHIIASLPHRFIPLFKQILSDRNTLSF |
| | | ILEEFKSDEEVIQSFCKYKTLLRNENVLETAEALFNELNSIDLTHIFISHKKLE |
| | | TISSALCDHWDTLRNALYERRISELTGKITKSAKEKVQRSLKHEDINLQEIIS |
| | | AAGKELSEAFKQKTSEILSHAHAALDQPLPTTLKKQEEKEILKSQLDSLLGL |
| | | YHLLDWFAVDESNEVDPEFSARLTGIKLEMEPSLSFYNKARNYATKKPYSV |
| | | EKFKLNFQMPTLARGWDVNREKNNGAILFVKNGLYYLGIMPKQKGRYKA |
| | | LSFEPTEKTSEGFDKMYYDYFPDAAKMIPKCSTQLKAVTAHFQTHTTPILLS |
| | | NNFIEPLEITKEIYDLNNPEKEPKKFQTAYAKKTGDQKGYREALCKWIDFTR |
| | | DFLSKYTKTTSIDLSSLRPSSQYKDLGEYYAELNPLLYHISFQRIAEKEIMDA |
| | | VETGKLYLFQIYNKDFAKGHHGKPNLHTLYWTGLFSPENLAKTSIKLNGQA |
| | | ELFYRPKSRMKRMAHRLGEKMLNKKLKDQKTPIPDTLYQELYDYVNHRLS |
| | | HDLSDEARALLPNVITKEVSHEIIKDRRFTSDKFFFHVPITLNYQAANSPSKF |
| | | NQRVNAYLKEHPETPIIGIARGERNLIYITVIDSTGKILEQRSLNTIQQFDYQK |
| | | KLDNREKERVAARQAWSVVGTIKDLKQGYLSQVIHEIVDLMIHYQAVVVL |
| | | ENLNFGFKSKRTGIAEKAVYQQFEKMLIDKLNCLVLKDYPAEKVGGVLNP |
| | | YQLTDQFTSFAKMGTQSGFLFYVPAPYTSKIDPLTGFVDPFVWKTIKNHESR |
| | | KHFLEGFDFLHYDVKTGDFILHFKMNRNLSFQRGLPGFMPAWDIVFEKNET |
| | | QFDAKGTPFIAGKRIVPVIENHRFTGRYRDLYPANELIALLEEKGIVFRDGSN |
| | | ILPKLLENDDSHAIDTMVALIRSVLQMRNSNAATGEDYINSPVRDLNGVCF |
| | | DSRFQNPEWPMDADANGAYHIALKGQLLLNHLKESKDLKLQNGISNQDWL |
| | | AYIQELRN |
| 20 | inactive | MTQFEGFTNLYQVSKTLRFELIPQGKTLKHIQEQGFIEEDKARNDHYKELKP |
| | HFAsCpf1 | IIDRIYKTYADQCLQLVQLDWENLSAAIDSYRKEKTEETRNALIEEQATYRN |
| | | AIHDYFIGRTDNLTDAINKRHAEIYKGLFKAELFNGKVLKQLGTVTTTEHEN |
| | | ALLRSFDKFTTYFSGFYRNRKNVFSAEDISTAIPHRIVQDNFPKFKENCHIFT |
| | | RLITAVPSLREHFENVKKAIGIFVSTSIEEVFSFPFYNQLLTQTQIDLYNQLLG |
| | | GISREAGTEKIKGLNEVLALAIQKNDETAHIIASLPHRFIPLFKQILSDRNTLSF |
| | | ILEEFKSDEEVIQSFCKYKTLLRNENVLETAEALFNELNSIDLTHIFISHKKLE |
| | | TISSALCDHWDTLRNALYERRISELTGKITKSAKEKVQRSLKHEDINLQEIIS |
| | | AAGKELSEAFKQKTSEILSHAHAALDQPLPTTLKKQEEKEILKSQLDSLLGL |
| | | YHLLDWFAVDESNEVDPEFSARLTGIKLEMEPSLSFYNKARNYATKKPYSV |
| | | EKFKLNFQMPTLARGWDVNREKNNGAILFVKNGLYYLGIMPKQKGRYKA |
| | | LSFEPTEKTSEGFDKMYYDYFPDAAKMIPKCSTQLKAVTAHFQTHTTPILLS |
| | | NNFIEPLEITKEIYDLNNPEKEPKKFQTAYAKKTGDQKGYREALCKWIDFTR |
| | | DFLSKYTKTTSIDLSSLRPSSQYKDLGEYYAELNPLLYHISFQRIAEKEIMDA |
| | | VETGKLYLFQIYNKDFAKGHHGKPNLHTLYWTGLFSPENLAKTSIKLNGQA |
| | | ELFYRPKSRMKRMAHRLGEKMLNKKLKDQKTPIPDTLYQELYDYVNHRLS |
| | | HDLSDEARALLPNVITKEVSHEIIKDRRFTSDKFFFHVPITLNYQAANSPSKF |
| | | NQRVNAYLKEHPETPIIGIARGERNLIYITVIDSTGKILEQRSLNTIQQFDYQK |
| | | KLDNREKERVAARQAWSVVGTIKDLKQGYLSQVIHEIVDLMIHYQAVVVL |
| | | ENLNFGFKSKRTGIAEKAVYQQFEKMLIDKLNCLVLKDYPAEKVGGVLNP |
| | | YQLTDQFTSFAKMGTQSGFLFYVPAPYTSKIDPLTGFVDPFVWKTIKNHESR |
| | | KHFLEGFDFLHYDVKTGDFILHFKMNRNLSFQRGLPGFMPAWDIVFEKNET |
| | | QFDAKGTPFIAGKRIVPVIENHRFTGRYRDLYPANELIALLEEKGIVFRDGSN |
| | | ILPKLLENDDSHAIDTMVALIRSVLQMRNSNAATGEDYINSPVRDLNGVCF |
| | | DSRFQNPEWPMDADANGAYHIALKGQLLLNHLKESKDLKLQNGISNQDWL |
| | | AYIQELRN |
| 21 | inactive | MTQFEGFTNLYQVSKTLRFELIPQGKTLKHIQEQGFIEEDKARNDHYKELKP |
| | RVRAsCpf1 | IIDRIYKTYADQCLQLVQLDWENLSAAIDSYRKEKTEETRNALIEEQATYRN |
| | | AIHDYFIGRTDNLTDAINKRHAEIYKGLFKAELFNGKVLKQLGTVTTTEHEN |
| | | ALLRSFDKFTTYFSGFYENRKNVFSAEDISTAIPHRIVQDNFPKFKENCHIFT |
| | | RLITAVPSLREHFENVKKAIGIFVSTSIEEVFSFPFYNQLLTQTQIDLYNQLLG |
| | | GISREAGTEKIKGLNEVLNLAIQKNDETAHIIASLPHRFIPLFKQILSDRNTLSF |
| | | ILEEFKSDEEVIQSFCKYKTLLRNENVLETAEALFNELNSIDLTHIFISHKKLE |
| | | TISSALCDHWDTLRNALYERRISELTGKITKSAKEKVQRSLKHEDINLQEIIS |
| | | AAGKELSEAFKQKTSEILSHAHAALDQPLPTTLKKQEEKEILKSQLDSLLGL |
| | | YHLLDWFAVDESNEVDPEFSARLTGIKLEMEPSLSFYNKARNYATKKPYSV |
| | | EKFKLNFQMPTLARGWDVNVEKNRGAILFVKNGLYYLGIMPKQKGRYKA |
| | | LSFEPTEKTSEGFDKMYYDYFPDAAKMIPKCSTQLKAVTAHFQTHTTPILLS |
| | | NNFIEPLEITKEIYDLNNPEKEPKKFQTAYAKKTGDQKGYREALCKWIDFTR |
| | | DFLSKYTKTTSIDLSSLRPSSQYKDLGEYYAELNPLLYHISFQRIAEKEIMDA |
| | | VETGKLYLFQIYNKDFAKGHHGKPNLHTLYWTGLFSPENLAKTSIKLNGQA |
| | | ELFYRPKSRMKRMAHRLGEKMLNKKLKDQKTPIPDTLYQELYDYVNHRLS |
| | | HDLSDEARALLPNVITKEVSHEIIKDRRFTSDKFFFHVPITLNYQAANSPSKF |
| | | NQRVNAYLKEHPETPIIGIARGERNLIYITVIDSTGKILEQRSLNTIQQFDYQK |
| | | KLDNREKERVAARQAWSVVGTIKDLKQGYLSQVIHEIVDLMIHYQAVVVL |
| | | ENLNFGFKSKRTGIAEKAVYQQFEKMLIDKLNCLVLKDYPAEKVGGVLNP |
| | | YQLTDQFTSFAKMGTQSGFLFYVPAPYTSKIDPLTGFVDPFVWKTIKNHESR |
| | | KHFLEGFDFLHYDVKTGDFILHFKMNRNLSFQRGLPGFMPAWDIVFEKNET |
| | | QFDAKGTPFIAGKRIVPVIENHRFTGRYRDLYPANELIALLEEKGIVFRDGSN |
| | | ILPKLLENDDSHAIDTMVALIRSVLQMRNSNAATGEDYINSPVRDLNGVCF |
| | | DSRFQNPEWPMDADANGAYHIALKGQLLLNHLKESKDLKLQNGISNQDWL |
| | | AYIQELRN |
| 22 | RRAsCpf1 | MTQFEGFTNLYQVSKTLRFELIPQGKTLKHIQEQGFIEEDKARNDHYKELKP |
| | | IIDRIYKTYADQCLQLVQLDWENLSAAIDSYRKEKTEETRNALIEEQATYRN |
| | | AIHDYFIGRTDNLTDAINKRHAEIYKGLFKAELFNGKVLKQLGTVTTTEHEN |
| | | ALLRSFDKFTTYFSGFYENRKNVFSAEDISTAIPHRIVQDNFPKFKENCHIFT |
| | | RLITAVPSLREHFENVKKAIGIFVSTSIEEVFSFPFYNQLLTQTQIDLYNQLLG |
| | | GISREAGTEKIKGLNEVLNLAIQKNDETAHIIASLPHRFIPLFKQILSDRNTLSF |
| | | ILEEFKSDEEVIQSFCKYKTLLRNENVLETAEALFNELNSIDLTHIFISHKKLE |
| | | TISSALCDHWDTLRNALYERRISELTGKITKSAKEKVQRSLKHEDINLQEIIS |
| | | AAGKELSEAFKQKTSEILSHAHAALDQPLPTTLKKQEEKEILKSQLDSLLGL |
| | | YHLLDWFAVDESNEVDPEFSARLTGIKLEMEPSLSFYNKARNYATKKPYSV |
| | | EKFKLNFQMPTLARGWDVNKEKNNGAILFVKNGLYYLGIMPKQKGRYKA |
| | | LSFEPTEKTSEGFDKMYYDYFPDAAKMIPRCSTQLKAVTAHFQTHTTPILLS |
| | | NNFIEPLEITKEIYDLNNPEKEPKKFQTAYAKKTGDQKGYREALCKWIDFTR |
| | | DFLSKYTKTTSIDLSSLRPSSQYKDLGEYYAELNPLLYHISFQRIAEKEIMDA |
| | | VETGKLYLFQIYNKDFAKGHHGKPNLHTLYWTGLFSPENLAKTSIKLNGQA |
| | | ELFYRPKSRMKRMAHRLGEKMLNKKLKDQKTPIPDTLYQELYDYVNHRLS |
| | | HDLSDEARALLPNVITKEVSHEIIKDRRFTSDKFFFHVPITLNYQAANSPSKF |
| | | NQRVNAYLKEHPETPIIGIARGERNLIYITVIDSTGKILEQRSLNTIQQFDYQK |
| | | KLDNREKERVAARQAWSVVGTIKDLKQGYLSQVIHEIVDLMIHYQAVVVL |
| | | ENLNFGFKSKRTGIAEKAVYQQFEKMLIDKLNCLVLKDYPAEKVGGVLNP |
| | | YQLTDQFTSFAKMGTQSGFLFYVPAPYTSKIDPLTGFVDPFVWKTIKNHESR |
| | | KHFLEGFDFLHYDVKTGDFILHFKMNRNLSFQRGLPGFMPAWDIVFEKNET |
| | | QFDAKGTPFIAGKRIVPVIENHRFTGRYRDLYPANELIALLEEKGIVFRDGSN |
| | | ILPKLLENDDSHAIDTMVALIRSVLQMRNSNAATGEDYINSPVRDLNGVCF |
| | | DSRFQNPEWPMDADANGAYHIALKGQLLLNHLKESKDLKLQNGISNQDWL |
| | | AYIQELRN |
| 23 | CasX | MEKRINKIRKKLSADNATKPVSRSGPMKTLLVRVMTDDLKKRLEKRRKKP |
| | | EVMPQVISNNAANNLRMLLDDYTKMKEAILQVYWQEFKDDHVGLMCKFA |
| | | QPASKKIDQNKLKPEMDEKGNLTTAGFACSQCGQPLFVYKLEQVSEKGKA |
| | | YTNYFGRCNVAEHEKLILLAQLKPEKDSDEAVTYSLGKFGQRALDFYSIHV |
| | | TKESTHPVKPLAQIAGNRYASGPVGKALSDACMGTIASFLSKYQDIIIEHQK |
| | | VVKGNQKRLESLRELAGKENLEYPSVTLPPQPHTKEGVDAYNEVIARVRM |
| | | WVNLNLWQKLKLSRDDAKPLLRLKGFPSFPVVERRENEVDWWNTINEVK |
| | | KLIDAKRDMGRVFWSGVTAEKRNTILEGYNYLPNENDHKKREGSLENPKK |
| | | PAKRQFGDLLLYLEKKYAGDWGKVFDEAWERIDKKIAGLTSHIEREEARN |
| | | AEDAQSKAVLTDWLRAKASFVLERLKEMDEKEFYACEIQLQKWYGDLRG |
| | | NPFAVEAENRVVDISGFSIGSDGHSIQYRNLLAWKYLENGKREFYLLMNYG |
| | | KKGRIRFTDGTDIKKSGKWQGLLYGGGKAKVIDLTFDPDDEQLIILPLAFGT |
| | | RQGREFIWNDLLSLETGLIKLANGRVIEKTIYNKKIGRDEPALFVALTFERRE |
| | | VVDPSNIKPVNLIGVDRGENIPAVIALTDPEGCPLPEFKDSSGGPTDILRIGEG |
| | | YKEKQRAIQAAKEVEQRRAGGYSRKFASKSRNLADDMVRNSARDLFYHA |
| | | VTHDAVLVFENLSRGFGRQGKRTFMTERQYTKMEDWLTAKLAYEGLTSK |
| | | TYLSKTLAQYTSKTCSNCGFTITTADYDGMLVRLKKTSDGWATTLNNKEL |
| | | KAEGQITYYNRYKRQTVEKELSAELDRLSEESGNNDISKWTKGRRDEALFL |
| | | LKKRFSHRPVQEQFVCLDCGHEVHADEQAALNIARSWLFLNSNSTEFKSYK |
| | | SGKQPFVGAWQAFYKRRLKEVWKPNA |
| 24 | dCasX | MEKRINKIRKKLSADNATKPVSRSGPMKTLLVRVMTDDLKKRLEKRRKKP |
| | | EVMPQVISNNAANNLRMLLDDYTKMKEAILQVYWQEFKDDHVGLMCKFA |
| | | QPASKKIDQNKLKPEMDEKGNLTTAGFACSQCGQPLFVYKLEQVSEKGKA |
| | | YTNYFGRCNVAEHEKLILLAQLKPEKDSDEAVTYSLGKFGQRALDFYSIHV |
| | | TKESTHPVKPLAQIAGNRYASGPVGKALSDACMGTIASFLSKYQDIIIEHQK |
| | | VVKGNQKRLESLRELAGKENLEYPSVTLPPQPHTKEGVDAYNEVIARVRM |
| | | WVNLNLWQKLKLSRDDAKPLLRLKGFPSFPVVERRENEVDWWNTINEVK |
| | | KLIDAKRDMGRVFWSGVTAEKRNTILEGYNYLPNENDHKKREGSLENPKK |
| | | PAKRQFGDLLLYLEKKYAGDWGKVFDEAWERIDKKIAGLTSHIEREEARN |
| | | AEDAQSKAVLTDWLRAKASFVLERLKEMDEKEFYACEIQLQKWYGDLRG |
| | | NPFAVEAENRVVDISGFSIGSDGHSIQYRNLLAWKYLENGKREFYLLMNYG |
| | | KKGRIRFTDGTDIKKSGKWQGLLYGGGKAKVIDLTFDPDDEQLIILPLAFGT |
| | | RQGREFIWNDLLSLETGLIKLANGRVIEKTIYNKKIGRDEPALFVALTFERRE |
| | | VVDPSNIKPVNLIGVARGENIPAVIALTDPEGCPLPEFKDSSGGPTDILRIGEG |
| | | YKEKQRAIQAAKEVEQRRAGGYSRKFASKSRNLADDMVRNSARDLFYHA |
| | | VTHDAVLVFANLSRGFGRQGKRTFMTERQYTKMEDWLTAKLAYEGLTSK |
| | | TYLSKTLAQYTSKTCSNCGFTITTADYDGMLVRLKKTSDGWATTLNNKEL |
| | | KAEGQITYYNRYKRQTVEKELSAELDRLSEESGNNDISKWTKGRRDEALFL |
| | | LKKRFSHRPVQEQFVCLDCGHEVHAAEQAALNIARSWLFLNSNSTEFKSYK |
| | | SGKQPFVGAWQAFYKRRLKEVWKPNA |
| 25 | CasY | MRKKLFKGYILHNKRLVYTGKAAIRSIKYPLVAPNKTALNNLSEKIIYDYEH |
| | | LFGPLNVASYARNSNRYSLVDFWIDSLRAGVIWQSKSTSLIDLISKLEGSKSP |
| | | SEKIFEQIDFELKNKLDKEQFKDIILLNTGIRSSSNVRSLRGRFLKCFKEEFRD |
| | | TEEVIACVDKWSKDLIVEGKSILVSKQFLYWEEEFGIKIFPHFKDNHDLPKLT |
| | | FFVEPSLEFSPHLPLANCLERLKKFDISRESLLGLDNNFSAFSNYFNELFNLLS |
| | | RGEIKKIVTAVLAVSKSWENEPELEKRLHFLSEKAKLLGYPKLTSSWADYR |
| | | MIIGGKIKSWHSNYTEQLIKVREDLKKHQIALDKLQEDLKKVVDSSLREQIE |
| | | AQREALLPLLDTMLKEKDFSDDLELYRFILSDFKSLLNGSYQRYIQTEEERK |
| | | EDRDVTKKYKDLYSNLRNIPRFFGESKKEQFNKFINKSLPTIDVGLKILEDIR |
| | | NALETVSVRKPPSITEEYVTKQLEKLSRKYKINAFNSNRFKQITEQVLRKYN |
| | | NGELPKISEVFYRYPRESHVAIRILPVKISNPRKDISYLLDKYQISPDWKNSNP |
| | | GEVVDLIEIYKLTLGWLLSCNKDFSMDFSSYDLKLFPEAASLIKNFGSCLSG |
| | | YYLSKMIFNCITSEIKGMITLYTRDKFVVRYVTQMIGSNQKFPLLCLVGEKQ |
| | | TKNFSRNWGVLIEEKGDLGEEKNQEKCLIFKDKTDFAKAKEVEIFKNNIWRI |
| | | RTSKYQIQFLNRLFKKTKEWDLMNLVLSEPSLVLEEEWGVSWDKDKLLPL |
| | | LKKEKSCEERLYYSLPLNLVPATDYKEQSAEIEQRNTYLGLDVGEFGVAYA |
| | | VVRIVRDRIELLSWGFLKDPALRKIRERVQDMKKKQVMAVFSSSSTAVARV |
| | | REMAIHSLRNQIHSIALAYKAKIIYEISISNFETGGNRMAKIYRSIKVSDVYRE |
| | | SGADTLVSEMIWGKKNKQMGNHISSYATSYTCCNCARTPFELVIDNDKEYE |
| | | KGGDEFIFNVGDEKKVRGFLQKSLLGKTIKGKEVLKSIKEYARPPIREVLLE |
| | | GEDVEQLLKRRGNSYIYRCPFCGYKTDADIQAALNIACRGYISDNAKDAVK |
| | | EGERKLDYILEVRKLWEKNGAVLRSAKFL |
| 26 | CasPhi | MADTPTLFTQFLRHHLPGQRFRKDILKQAGRILANKGEDATIAFLRGKSEES |
| | | PPDFQPPVKCPIIACSRPLTEWPIYQASVAIQGYVYGQSLAEFEASDPGCSKD |
| | | GLLGWFDKTGVCTDYFSVQGLNLIFQNARKRYIGVQTKVTNRNEKRHKKL |
| | | KRINAKRIAEGLPELTSDEPESALDETGHLIDPPGLNTNIYCYQQVSPKPLAL |
| | | SEVNQLPTAYAGYSTSGDDPIQPMVTKDRLSISKGQPGYIPEHQRALLSQKK |
| | | HRRMRGYGLKARALLVIVRIQDDWAVIDLRSLLRNAYWRRIVQTKEPSTIT |
| | | KLLKLVTGDPVLDATRMVATFTYKPGIVQVRSAKCLKNKQGSKLFSERYL |
| | | NETVSVTSIDLGSNNLVAVATYRLVNGNTPELLQRFTLPSHLVKDFERYKQ |
| | | AHDTLEDSIQKTAVASLPQGQQTEIRMWSMYGFREAQERVCQELGLADGSI |
| | | PWNVMTATSTILTDLFLARGGDPKKCMFTSEPKKKKNSKQVLYKIRDRAW |
| | | AKMYRTLLSKETREAWNKALWGLKRGSPDYARLSKRKEELARRCVNYTIS |
| | | TAEKRAQCGRTIVALEDLNIGFFHGRGKQEPGWVGLFTRKKENRWLMQAL |
| | | HKAFLELAHHRGYHVIEVNPAYTSQTCPVCRHCDPDNRDQHNREAFHCIGC |
| | | GFRGNADLDVATHNIAMVAITGESLKRARGSVASKTPQPLAAE |
| 27 | dCasPhi | MPKPAVESEFSKVLKKHFPGERFRSSYMKRGGKILAAQGEEAVVAYLQGK |
| | | SEEEPPNFQPPAKCHVVTKSRDFAEWPIMKASEAIQRYIYALSTTERAACKP |
| | | GKSSESHAAWFAATGVSNHGYSHVQGLNLIFDHTLGRYDGVLKKVQLRNE |
| | | KARARLESINASRADEGLPEIKAEEEEVATNETGHLLQPPGINPSFYVYQTIS |
| | | PQAYRPRDEIVLPPEYAGYVRDPNAPIPLGVVRNRCDIQKGCPGYIPEWQRE |
| | | AGTAISPKTGKAVTVPGLSPKKNKRMRRYWRSEKEKAQDALLVTVRIGTD |
| | | WVVIDVRGLLRNARWRTIAPKDISLNALLDLFTGDPVIDVRRNIVTFTYTLD |
| | | ACGTYARKWTLKGKQTKATLDKLTATQTVALVAIALGQTNPISAGISRVTQ |
| | | ENGALQCEPLDRFTLPDDLLKDISAYRIAWDRNEEELRARSVEALPEAQQA |
| | | EVRALDGVSKETARTQLCADFGLDPKRLPWDKMSSNTTFISEALLSNSVSR |
| | | DQVFFTPAPKKGAKKKAPVEVMRKDRTWARAYKPRLSVEAQKLKNEALW |
| | | ALKRTSPEYLKLSRRKEELCRRSINYVIEKTRRRTQCQIVIPVIEDLNVRFFH |
| | | GSGKRLPGWDNFFTAKKENRWFIQGLHKAFSDLRTHRSFYVFEVRPERTSIT |
| | | CPKCGHCEVGNRDGEAFQCLSCGKTCNADLDVATHNLTQVALTGKTMPK |
| | | REEPRDAQGTAPARKTKKASKSKAPPAEREDQTPAQEPSQTS |
| 28 | Cas12f1 | MIKVYRYEIVKPLDLDWKEFGTILRQLQQETRFALNKATQLAWEWMGFSS |
| | (Cas14a) | DYKDNHGEYPKSKDILGYTNVHGYAYHTIKTKAYRLNSGNLSQTIKRATD |
| | | RFKAYQKEILRGDMSIPSYKRDIPLDLIKENISVNRMNHGDYIASLSLLSNPA |
| | | KQEMNVKRKISVIIIVRGAGKTIMDRILSGEYQVSASQIIHDDRKNKWYLNIS |
| | | YDFEPQTRVLDLNKIMGIDLGVAVAVYMAFQHTPARYKLEGGEIENFRRQ |
| | | VESRRISMLRQGKYAGGARGGHGRDKRIKPIEQLRDKIANFRDTTNHRYSR |
| | | YIVDMAIKEGCGTIQMEDLTNIRDIGSRFLQNWTYYDLQQKIIYKAEEAGIK |
| | | VIKIDPQYTSQRCSECGNIDSGNRIGQAIFKCRACGYEANADYNAARNIAIPN |
| | | IDKIIAESIKSGGS |
| 29 | Cas12f2 | NAMIAQKTIKIKLNPTKEQIIKLNSIIEEYIKVSNFTAKKIAEIQESFTDSGLTQ |
| | (Cas14b) | GTCSECGKEKTYRKYHLLKKDNKLFCITCYKRKYSQFTLQKVEFQNKTGLR |
| | | NVAKLPKTYYTNAIRFASDTFSGFDEIIKKKQNRLNSIQNRLNFWKELLYNP |
| | | SNRNEIKIKVVKYAPKTDTREHPHYYSEAEIKGRIKRLEKQLKKFKMPKYPE |
| | | FTSETISLQRELYSWKNPDELKISSITDKNESMNYYGKEYLKRYIDLINSQTP |
| | | QILLEKENNSFYLCFPITKNIEMPKIDDTFEPVGIDWGITRNIAVVSILDSKTK |
| | | KPKFVKFYSAGYILGKRKHYKSLRKHFGQKKRQDKINKLGTKEDRFIDSNI |
| | | HKLAFLIVKEIRNHSNKPIILMENITDNREEAEKSMRQNILLHSVKSRLQNYI |
| | | AYKALWNNIPTNLVKPEHTSQICNRCGHQDRENRPKGSKLFKCVKCNYMS |
| | | NADFNASINIARKFYIGEYEPFYKDNEKMKSGVNSISM |
| 30 | Cas12f3 | MEVQKTVMKTLSLRILRPLYSQEIEKEIKEEEKERRKQAGGTGELDGGFYK |
| | (Cas14c) | KLEKKHSEMFSFDRLNLLLNQLQREIAKVYNHAISELYIATIAQGNKSNKHY |
| | | ISSIVYNRAYGYFYNAYIALGICSKVEANFRSNELLTQQSALPTAKSDNFPIV |
| | | LHKQKGAEGEDGGFRISTEGSDLIFEIPIPFYEYNGENRKEPYKWVKKGGQK |
| | | PVLKLILSTFRRQRNKGWAKDEGTDAEIRKVTEGKYQVSQIEINRGKKLGE |
| | | HQKWFANFSIEQPIYERKPNRSIVGGLDVGIRSPLVCAINNSFSRYSVDSNDV |
| | | FKFSKQVFAFRRRLLSKNSLKRKHGHAAHKLEPITEMTEKNDKFRKKIIER |
| | | WAKEVTNFFVKNQVGIVQIEDLSTMKDREDHFFNQYLRGFWPYYQMQTLI |
| | | ENKLKEYGIEVKRVQAKYTSQLCSNPNCRYWNNYFNFEYRKVNKFPKFKC |
| | | EKCNLEISADYNAARNLSTPDIEKFVAKATKGINLPEK |
| 31 | C2c8 | MKVLEFKIHPTEEQVSKIDQSLAACKLLWNLSIALKEESKQRYYRKKHKFD |
| | | EFSPEIWGLSYSGHYDEKEFKTLKDKEKKLLIGNPCCKIAYFKKTSNGKEYT |
| | | PLNSIPIRRFMNAENIDKDAVNYLNRKKLAFYFRENTAKFIGEIETEFKKGFF |
| | | KSVIKPAYDAAKKGIRGIPRFKGRRDKVETLVNGQPETIKIKSNGVIVSSKIG |
| | | LLKIRGLDRLQGKAPRMAKITRKATGYYLQLTIETDDTIYKESDKCVGLDM |
| | | GAVAIFTDDLGRQSEAKRYAKIQKKRLNRLQRQASRQKDNSNNQRKTYAK |
| | | LARVHEKIARQRKGRNAQLAHKITSEYQSVILEDLNLKNMTAAAKPKERED |
| | | GDGYKQNGKKRKSGLNKALLDNAIGQLRTFIENKANERGRKIIRVNPKHTS |
| | | QTCPNCGNIDKANRVSQSKFKCVSCGYEAHADQNAAANILIRGLRDEFLRA |
| | | IGSLYKFPVSMIGKYPGLAGEFTPDLDANQESIGDAPIENAEHSISKQMKQE |
| | | GNRTPTQPENGSQSLIFLSAPPQPCGDSHGTNNPKALPNKASKRSSKKPRGA |
| | | IPENPDQLTIWDLLD |
| 32 | human | MPARTAPARVPTLAVPAISLPDDVRRRLKDLERDSLTEKECVKEKLNLLHE |
| | DNMT1 | FLQTEIKNQLCDLETKLRKEELSEEGYLAKVKSLLNKDLSLENGAHAYNRE |
| | | VNGRLENGNQARSEARRVGMADANSPPKPLSKPRTPRRSKSDGEAKPEPSP |
| | | SPRITRKSTRQTTITSHFAKGPAKRKPQEESERAKSDESIKEEDKDQDEKRRR |
| | | VTSRERVARPLPAEEPERAKSGTRTEKEEERDEKEEKRLRSQTKEPTPKQKL |
| | | KEEPDREARAGVQADEDEDGDEKDEKKHRSQPKDLAAKRRPEEKEPEKVN |
| | | PQISDEKDEDEKEEKRRKTTPKEPTEKKMARAKTVMNSKTHPPKCIQCGQY |
| | | LDDPLKYGQHPPDAVDEPQMLTNEKLSIFDANESGFESYEALPQHKLTCFS |
| | | VYCKHGHLCPIDTGLIEKNIELFFSGSAKPIYDDDPSLEGGVNGKNLGPINE |
| | | WWITGFDGGEKALIGFSTSFAEYILMDPSPEYAPIFGLMQEKIYISKIVVEFL |
| | | QSNSDSTYEDLINKIETTVPPSGLNLNRFTEDSLLRHAQFVVEQVESYDEAG |
| | | DSDEQPIFLTPCMRDLIKLAGVTLGQRRAQARRQTIRHSTREKDRGPTKATT |
| | | TKLVYQIFDTFFAEQIEKDDREDKENAFKRRRCGVCEVCQQPECGKCKACK |
| | | DMVKFGGSGRSKQACQERRCPNMAMKEADDDEEVDDNIPEMPSPKKMHQ |
| | | GKKKKQNKNRISWVGEAVKTDGKKSYYKKVCIDAETLEVGDCVSVIPDDS |
| | | SKPLYLARVTALWEDSSNGQMFHAHWFCAGTDTVLGATSDPLELFLVDEC |
| | | EDMQLSYIHSKVKVIYKAPSENWAMEGGMDPESLLEGDDGKTYFYQLWY |
| | | DQDYARFESPPKTQPTEDNKFKFCVSCARLAEMRQKEIPRVLEQLEDLDSR |
| | | VLYYSATKNGILYRVGDGVYLPPEAFTFNIKLSSPVKRPRKEPVDEDLYPEH |
| | | YRKYSDYIKGSNLDAPEPYRIGRIKEIFCPKKSNGRPNETDIKIRVNKFYRPE |
| | | NTHKSTPASYHADINLLYWSDEEAVVDFKAVQGRCTVEYGEDLPECVQVY |
| | | SMGGPNRFYFLEAYNAKSKSFEDPPNHARSPGNKGKGKGKGKGKPKSQAC |
| | | EPSEPEIEIKLPKLRTLDVFSGCGGLSEGFHQAGISDTLWAIEMWDPAAQAF |
| | | RLNNPGSTVFTEDCNILLKLVMAGETTNSRGQRLPQKGDVEMLCGGPPCQ |
| | | GFSGMNRFNSRTYSKFKNSLVVSFLSYCDYYRPRFFLLENVRNFVSFKRSM |
| | | VLKLTLRCLVRMGYQCTFGVLQAGQYGVAQTRRRAIILAAAPGEKLPLFPE |
| | | PLHVFAPRACQLSVVVDDKKFVSNITRLSSGPFRTITVRDTMSDLPEVRNGA |
| | | SALEISYNGEPQSWFQRQLRGAQYQPILRDHICKDMSALVAARMRHIPLAP |
| | | GSDWRDLPNIEVRLSDGTMARKLRYTHHDRKNGRSSSGALRGVCSCVEAG |
| | | KACDPAARQFNTLIPWCLPHTGNRHNHWAGLYGRLEWDGFFSTTVTNPEP |
| | | MGKQGRVLHPEQHRVVSVRECARSQGFPDTYRLFGNILDKHRQVGNAVPP |
| | | PLAKAIGLEIKLCMLAKARESASAKIKEEEAAKD |
| 33 | human | MPAMPSSGPGDTSSSAAEREEDRKDGEEQEEPRGKEERQEPSTTARKVGRP |
| | DNMT3A | GRKRKHPPVESGDTPKDPAVISKSPSMAQDSGASELLPNGDLEKRSEPQPEE |
| | | GSPAGGQKGGAPAEGEGAAETLPEASRAVENGCCTPKEGRGAPAEAGKEQ |
| | | KETNIESMKMEGSRGRLRGGLGWESSLRQRPMPRLTFQAGDPYYISKRKRD |
| | | EWLARWKREAEKKAKVIAGMNAVEENQGPGESQKVEEASPPAVQQPTDP |
| | | ASPTVATTPEPVGSDAGDKNATKAGDDEPEYEDGRGFGIGELVWGKLRGF |
| | | SWWPGRIVSWWMTGRSRAAEGTRWVMWFGDGKFSVVCVEKLMPLSSFC |
| | | SAFHQATYNKQPMYRKAIYEVLQVASSRAGKLFPVCHDSDESDTAKAVEV |
| | | QNKPMIEWALGGFQPSGPKGLEPPEEEKNPYKEVYTDMWVEPEAAAYAPP |
| | | PPAKKPRKSTAEKPKVKEIIDERTRERLVYEVRQKCRNIEDICISCGSLNVTL |
| | | EHPLFVGGMCQNCKNCFLECAYQYDDDGYQSYCTICCGGREVLMCGNNN |
| | | CCRCFCVECVDLLVGPGAAQAAIKEDPWNCYMCGHKGTYGLLRRREDWP |
| | | SRLQMFFANNHDQEFDPPKVYPPVPAEKRKPIRVLSLFDGIATGLLVLKDLG |
| | | IQVDRYIASEVCEDSITVGMVRHQGKIMYVGDVRSVTQKHIQEWGPFDLVI |
| | | GGSPCNDLSIVNPARKGLYEGTGRLFFEFYRLLHDARPKEGDDRPFFWLFE |
| | | NVVAMGVSDKRDISRFLESNPVMIDAKEVSAAHRARYFWGNLPGMNRPLA |
| | | STVNDKLELQECLEHGRIAKFSKVRTITTRSNSIKQGKDQHFPVFMNEKEDI |
| | | LWCTEMERVFGFPVHYTDVSNMSRLARQRLLGRSWSVPVIRHLFAPLKEYF |
| | | ACV |
| 34 | human | NHDQEFDPPKVYPPVPAEKRKPIRVLSLFDGIATGLLVLKDLGIQVDRYIASE |
| | DNMT3A | VCEDSITVGMVRHQGKIMYVGDVRSVTQKHIQEWGPFDLVIGGSPCNDLSI |
| | catalytic | VNPARKGLYEGTGRLFFEFYRLLHDARPKEGDDRPFFWLFENVVAMGVSD |
| | domain | KRDISRFLESNPVMIDAKEVSAAHRARYFWGNLPGMNRPLASTVNDKLEL |
| | | QECLEHGRIAKFSKVRTITTRSNSIKQGKDQHFPVFMNEKEDILWCTEMERV |
| | | FGFPVHYTDVSNMSRLARQRLLGRSWSVPVIRHLFAPLKEYFACV |
| 35 | human | MKGDTRHLNGEEDAGGREDSILVNGACSDQSSDSPPILEAIRTPEIRGRRSSS |
| | DNMT3B | RLSKREVSSLLSYTQDLTGDGDGEDGDGSDTPVMPKLFRETRTRSESPAVR |
| | | TRNNNSVSSRERHRPSPRSTRGRQGRNHVDESPVEFPATRSLRRRATASAGT |
| | | PWPSPPSSYLTIDLTDDTEDTHGTPQSSSTPYARLAQDSQQGGMESPQVEAD |
| | | SGDGDSSEYQDGKEFGIGDLVWGKIKGFSWWPAMVVSWKATSKRQAMSG |
| | | MRWVQWFGDGKFSEVSADKLVALGLFSQHFNLATFNKLVSYRKAMYHAL |
| | | EKARVRAGKTFPSSPGDSLEDQLKPMLEWAHGGFKPTGIEGLKPNNTQPVV |
| | | NKSKVRRAGSRKLESRKYENKTRRRTADDSATSDYCPAPKRLKTNCYNNG |
| | | KDRGDEDQSREQMASDVANNKSSLEDGCLSCGRKNPVSFHPLFEGGLCQT |
| | | CRDRFLELFYMYDDDGYQSYCTVCCEGRELLLCSNTSCCRCFCVECLEVLV |
| | | GTGTAAEAKLQEPWSCYMCLPQRCHGVLRRRKDWNVRLQAFFTSDTGLE |
| | | YEAPKLYPAIPAARRRPIRVLSLFDGIATGYLVLKELGIKVGKYVASEVCEES |
| | | IAVGTVKHEGNIKYVNDVRNITKKNIEEWGPFDLVIGGSPCNDLSNVNPAR |
| | | KGLYEGTGRLFFEFYHLLNYSRPKEGDDRPFFWMFENVVAMKVGDKRDIS |
| | | RFLECNPVMIDAIKVSAAHRARYFWGNLPGMNRPVIASKNDKLELQDCLE |
| | | YNRIAKLKKVQTITTKSNSIKQGKNQLFPVVMNGKEDVLWCTELERIFGFP |
| | | VHYTDVSNMGRGARQKLLGRSWSVPVIRHLFAPLKDYFACE |
| 36 | mouse | MRGGSRHLSNEEDVSGCEDCIIISGTCSDQSSDPKTVPLTQVLEAVCTVENR |
| | DNMT3C | GCRTSSQPSKRKASSLISYVQDLTGDGDEDRDGEVGGSSGSGTPVMPQLFC |
| | | ETRIPSKTPAPLSWQANTSASTPWLSPASPYPIIDLTDEDVIPQSISTPSVDWS |
| | | QDSHQEGMDTTQVDAESRDGGNIEYQVSADKLLLSQSCILAAFYKLVPYRE |
| | | SIYRTLEKARVRAGKACPSSPGESLEDQLKPMLEWAHGGFKPTGIEGLKPN |
| | | KKQPENKSRRRTTNDPAASESSPPKRLKTNSYGGKDRGEDEESREQMASDV |
| | | TNNKGNLEDHCLSCGRKDPVSFHPLFEGGLCQSCRDRFLELFYMYDEDGY |
| | | QSYCTVCCEGRELLLCSNTSCCRCFCVECLEVLVGAGTAEDVKLQEPWSCY |
| | | MCLPQRCHGVLRRRKDWNMRLQDFFTTDPDLEEFEPPKLYPAIPAAKRRPI |
| | | RVLSLFDGIATGYLVLKELGIKVEKYIASEVCAESIAVGTVKHEGQIKYVDD |
| | | IRNITKEHIDEWGPFDLVIGGSPCNDLSCVNPVRKGLFEGTGRLFFEFYRLLN |
| | | YSCPEEEDDRPFFWMFENVVAMEVGDKRDISRFLECNPVMIDAIKVSAAHR |
| | | ARYFWGNLPGMNRPVMASKNDKLELQDCLEFSRTAKLKKVQTITTKSNSIR |
| | | QGKNQLFPVVMNGKDDVLWCTELERIFGFPEHYTDVSNMGRGARQKLLG |
| | | RSWSVPVIRHLFAPLKDHFACE |
| 37 | human | MAAIPALDPEAEPSMDVILVGSSELSSSVSPGTGRDLIAYEVKANQRNIEDIC |
| | DNMT3L | ICCGSLQVHTQHPLFEGGICAPCKDKFLDALFLYDDDGYQSYCSICCSGETL |
| | | LICGNPDCTRCYCFECVDSLVGPGTSGKVHAMSNWVCYLCLPSSRSGLLQR |
| | | RRKWRSQLKAFYDRESENPLEMFETVPVWRRQPVRVLSLFEDIKKELTSLG |
| | | FLESGSDPGQLKHVVDVTDTVRKDVEEWGPFDLVYGATPPLGHTCDRPPS |
| | | WYLFQFHRLLQYARPKPGSPRPFFWMFVDNLVLNKEDLDVASRFLEMEPV |
| | | TIPDVHGGSLQNAVRVWSNIPAIRSSRHWALVSEEELSLLAQNKQSSKLAA |
| | | KWPTKLVKNCFLPLREYFKYFSTELTSSL |
| 38 | human | NPLEMFETVPVWRRQPVRVLSLFEDIKKELTSLGFLESGSDPGQLKHVVDV |
| | DNMT3L | TDTVRKDVEEWGPFDLVYGATPPLGHTCDRPPSWYLFQFHRLLQYARPKP |
| | catalytic | GSPRPFFWMFVDNLVLNKEDLDVASRFLEMEPVTIPDVHGGSLQNAVRVW |
| | domain | SNIPAIRSRHWALVSEEELSLLAQNKQSSKLAAKWPTKLVKNCFLPLREYFK |
| | | YFSTELTSSL |
| 39 | mouse | MGSRETPSSCSKTLETLDLETSDSSSPDADSPLEEQWLKSSPALKEDSVDVV |
| | DNMT3L | LEDCKEPLSPSSPPTGREMIRYEVKVNRRSIEDICLCCGTLQVYTRHPLFEGG |
| | | LCAPCKDKFLESLFLYDDDGHQSYCTICCSGGTLFICESPDCTRCYCFECVDI |
| | | LVGPGTSERINAMACWVCFLCLPFSRSGLLQRRKRWRHQLKAFHDQEGAG |
| | | PMEIYKTVSAWKRQPVRVLSLFRNIDKVLKSLGFLESGSGSGGGTLKYVED |
| | | VTNVVRRDVEKWGPFDLVYGSTQPLGSSCDRCPGWYMFQFHRILQYALPR |
| | | QESQRPFFWIFMDNLLLTEDDQETTTRFLQTEAVTLQDVRGRDYQNAMRV |
| | | WSNIPGLKSKHAPLTPKEEEYLQAQVRSRSKLDAPKVDLLVKNCLLPLREY |
| | | FKYFSQNSLPL |
| 40 | mouse | GPMEIYKTVSAWKRQPVRVLSLFRNIDKVLKSLGFLESGSGSGGGTLKYVE |
| | DNMT3L | DVTNVVRRDVEKWGPFDLVYGSTQPLGSSCDRCPGWYMFQFHRILQYALP |
| | catalytic | RQESQRPFFWIFMDNLLLTEDDQETTTRFLQTEAVTLQDVRGRDYQNAMR |
| | domain | VWSNIPGLKSKHAPLTPKEEEYLQAQVRSRSKLDAPKVDLLVKNCLLPLRE |
| | | YFKYFSQNSLPL |
| 41 | human | MEPLRVLELYSGVGGMHHALRESCIPAQVVAAIDVNTVANEVYKYNFPHT |
| | TRDMT1 | QLLAKTIEGITLEEFDRLSFDMILMSPPCQPFTRIGRQGDMTDSRTNSFLHILD |
| | (DNMT2) | ILPRLQKLPKYILLENVKGFEVSSTRDLLIQTIENCGFQYQEFLLSPTSLGIPNS |
| | | RLRYFLIAKLQSEPLPFQAPGQVLMEFPKIESVHPQKYAMDVENKIQEKNVE |
| | | PNISFDGSIQCSGKDAILFKLETAEEIHRKNQQDSDLSVKMLKDFLEDDTDV |
| | | NQYLLPPKSLLRYALLLDIVQPTCRRSVCFTKGYGSYIEGTGSVLQTAEDVQ |
| | | VENIYKSLTNLSQEEQITKLLILKLRYFTPKEIANLLGFPPEFGFPEKITVKQR |
| | | YRLLGNSLNVHVVAKLIKILYE |
| 42 | M. | MNSNKDKIKVIKVFEAFAGIGSQFKALKNIARSKNWEIQHSGMVEWFVDAI |
| | penetrans | VSYVAIHSKNFNPKIEQLDKDILSISNDSKMPISEYGIKKINNTIKASYLNYAK |
| | M MpeI | KHFNNLFDIKKVNKDNFPKNIDIFTYSFPCQDLSVQGLQKGIDKELNTRSGL |
| | | LWEIERILEEIKNSFSKEEMPKYLLMENVKNLLSHKNKKNYNTWLKQLEKF |
| | | GYKSKTYLLNSKNFDNCQNRERVFCLSIRDDYLEKTGFKFKELEKVKNPPK |
| | | KIKDILVDSSNYKYLNLNKYETTTFRETKSNIISRSLKNYTTFNSENYVYNIN |
| | | GIGPTLTASGANSRIKIETQQGVRYLTPLECFKYMQFDVNDFKKVQSTNLIS |
| | | ENKMIYIAGNSIPVKILEAIFNTLEFVNNEE |
| 43 | S. | MSKVENKTKKLRVFEAFAGIGAQRKALEKVRKDEYEIVGLAEWYVPAIVM |
| | monobiae | YQAIHNNFHTKLEYKSVSREEMIDYLENKTLSWNSKNPVSNGYWKRKKDD |
| | M SssI | ELKIIYNAIKLSEKEGNIFDIRDLYKRTLKNIDLLTYSFPCQDLSQQGIQKGM |
| | | KRGSGTRSGLLWEIERALDSTEKNDLPKYLLMENVGALLHKKNEEELNQW |
| | | KQKLESLGYQNSIEVLNAADFGSSQARRRVFMISTLNEFVELPKGDKKPKSI |
| | | KKVLNKIVSEKDILNNLLKYNLTEFKKTKSNINKASLIGYSKFNSEGYVYDP |
| | | EFTGPTLTASGANSRIKIKDGSNIRKMNSDETFLYIGFDSQDGKRVNEIEFLT |
| | | ENQKIFVCGNSISVEVLEAIIDKIGG |
| 44 | H. | MKDVLDDNLLEEPAAQYSLFEPESNPNLREKFTFIDLFAGIGGFRIAMQNLG |
| | parainfluenzae | GKCIFSSEWDEQAQKTYEANFGDLPYGDITLEETKAFIPEKFDILCAGFPCQA |
| | M HpaII | FSIAGKRGGFEDTRGTLFFDVAEIIRRHQPKAFFLENVKGLKNHDKGRTLKT |
| | | ILNVLREDLGYFVPEPAIVNAKNFGVPQNRERIYIVGFHKSTGVNSFSYPEPL |
| | | DKIVTFADIREEKTVPTKYYLSTQYIDTLRKHKERHESKGNGFGYEIIPDDGI |
| | | ANAIVVGGMGRERNLVIDHRITDFTPTTNIKGEVNREGIRKMTPREWARLQ |
| | | GFPDSYVIPVSDASAYKQFGNSVAVPAIQATGKKILEKLGNLYD |
| 45 | A. luteus | MSKANAKYSFVDLFAGIGGFHAALAATGGVCEYAVEIDREAAAVYERNW |
| | M AluI | NKPALGDITDDANDEGVTLRGYDGPIDVLTGGFPCQPFSKSGAQHGMAETR |
| | | GTLFWNIARIIEEREPTVLILENVRNLVGPRHRHEWLTIIETLRFFGYEVSGAP |
| | | AIFSPHLLPAWMGGTPQVRERVFITATLVPERMRDERIPRTETGEIDAEAIGP |
| | | KPVATMNDRFPIKKGGTELFHPGDRKSGWNLLTSGIIREGDPEPSNVDLRLT |
| | | ETETLWIDAWDDLESTIRRATGRPLEGFPYWADSWTDFRELSRLVVIRGFQ |
| | | APEREVVGDRKRYVARTDMPEGFVPASVTRPAIDETLPAWKQSHLRRNYD |
| | | FFERHFAEVVAWAYRWGVYTDLFPASRRKLEWQAQDAPRLWDTVMHFRP |
| | | SGIRAKRPTYLPALVAITQTSIVGPLERRLSPRETARLQGLPEWFDFGEQRAA |
| | | ATYKQMGNGVNVGVVRHILREHVRRDRALLKLTPAGQRIINAVLADEPDA |
| | | TVGALGAAE |
| 46 | H. | MNLISLFSGAGGLDLGFQKAGFRIICANEYDKSIWKTYESNHSAKLIKGDIS |
| | aegyptius | KISSDEFPKCDGIIGGPPCQSWSEGGSLRGIDDPRGKLFYEYIRILKQKKPIFF |
| | M HaeIII | LAENVKGMMAQRHNKAVQEFIQEFDNAGYDVHIILLNANDYGVAQDRKR |
| | | VFYIGFRKELNINYLPPIPHLIKPTFKDVIWDLKDNPIPALDKNKTNGNKCIY |
| | | PNHEYFIGSYSTIFMSRNRVRQWNEPAFTVQASGRQCQLHPQAPVMLKVSK |
| | | NLNKFVEGKEHLYRRLTVRECARVQGFPDDFIFHYESLNDGYKMIGNAVPV |
| | | NLAYEIAKTIKSALEICKGN |
| 47 | H. | MIEIKDKQLTGLRFIDLFAGLGGFRLALESCGAECVYSNEWDKYAQEVYEM |
| | haemolyticus | NFGEKPEGDITQVNEKTIPDHDILCAGFPCQAFSISGKQKGFEDSRGTLFFDI |
| | M HhaI | ARIVREKKPKVVFMENVKNFASHDNGNTLEVVKNTMNELDYSFHAKVLN |
| | | ALDYGIPQKRERIYMICFRNDLNIQNFQFPKPFELNTFVKDLLLPDSEVEHLV |
| | | IDRKDLVMTNQEIEQTTPKTVRLGIVGKGGQGERIYSTRGIAITLSAYGGGIF |
| | | AKTGGYLVNGKTRKLHPRECARVMGYPDSYKVHPSTSQAYKQFGNSVVIN |
| | | VLQYIAYNIGSSLNFKPY |
| 48 | Moraxella | MKPEILKLIRSKLDLTQKQASEIIEVSDKTWQQWESGKTEMHPAYYSFLQE |
| | M MspI | KLKDKINFEELSAQKTLQKKIFDKYNQNQITKNAEELAEITHIEERKDAYSS |
| | | DFKFIDLFSGIGGIRQSFEVNGGKCVFSSEIDPFAKFTYYTNFGVVPFGDITKV |
| | | EATTIPQHDILCAGFPCQPFSHIGKREGFEHPTQGTMFHEIVRIIETKKTPVLF |
| | | LENVPGLINHDDGNTLKVIIETLEDMGYKVHHTVLDASHFGIPQKRKRFYL |
| | | VAFLNQNIHFEFPKPPMISKDIGEVLESDVTGYSISEHLQKSYLFKKDDGKPS |
| | | LIDKNTTGAVKTLVSTYHKIQRLTGTFVKDGETGIRLLTTNECKAIMGFPKD |
| | | FVIPVSRTQMYRQMGNSVVVPVVTKIAEQISLALKTVNQQSPQENFELELV |
| 49 | Ascobolus | MSERRYEAGMTVALHEGSFLKIQRVYIRQYHADNRREHMLVGPLFRRTKY |
| | Masc1 | LKALSKKVNEVAIVHESIHVPVQDVIGVRELIITNRPFPECRKGDEHTGRLVC |
| | | RWVYNLDERAKGREYKKQRYIRRITEAEADPEYRVEDRVLRRRWFQEGYI |
| | | GDEISYKEHGNGDIVDIRSESPLQVLDGWGGDLVDLENGEETSIPGPCRSAS |
| | | SYGRLMKPPLAQAADSNTSRKYTFGDTFCGGGGVSLGARQAGLEVKWAF |
| | | DMNPNAGANYRRNFPNTDFFLAEAEQFIQLSVGISQHVDILHLSPPCQTFSR |
| | | AHTIAGKNDENNEASFFAVVNLIKAVRPRLFTVEETDGIMDRQSRQFIDTAL |
| | | MGITELGYSFRICVLNAIEYGVCQNRKRLIIIGAAPGEELPPFPLPTHQDFFSK |
| | | DPRRDLLPAVTLDDALSTITPESTDHHLNHVWQPAEWKTPYDAHRPFKNAI |
| | | RAGGGEYDIYPDGRRKFTVRELACIQGFPDEYEFVGTLTDKRRIIGNAVPPP |
| | | LSAAIMSTLRQWMTEKDFERME |
| 50 | Arabidopsis | MVENGAKAAKRKKRPLPEIQEVEDVPRTRRPRRAAACTSFKEKSIRVCEKS |
| | MET1 | ATIEVKKQQIVEEEFLALRLTALETDVEDRPTRRLNDFVLFDSDGVPQPLEM |
| | | LEIHDIFVSGAILPSDVCTDKEKEKGVRCTSFGRVEHWSISGYEDGSPVIWIS |
| | | TELADYDCRKPAASYRKVYDYFYEKARASVAVYKKLSKSSGGDPDIGLEE |
| | | LLAAVVRSMSSGSKYFSSGAAIIDFVISQGDFIYNQLAGLDETAKKHESSYV |
| | | EIPVLVALREKSSKIDKPLQRERNPSNGVRIKEVSQVAESEALTSDQLVDGT |
| | | DDDRRYAILLQDEENRKSMQQPRKNSSSGSASNMFYIKINEDEIANDYPLPS |
| | | YYKTSEEETDELILYDASYEVQSEHLPHRMLHNWALYNSDLRFISLELLPM |
| | | KQCDDIDVNIFGSGVVTDDNGSWISLNDPDSGSQSHDPDGMCIFLSQIKEW |
| | | MIEFGSDDIISISIRTDVAWYRLGKPSKLYAPWWKPVLKTARVGISILTFLRV |
| | | ESRVARLSFADVTKRLSGLQANDKAYISSDPLAVERYLVVHGQIILQLFAVY |
| | | PDDNVKRCPFVVGLASKLEDRHHTKWIIKKKKISLKELNLNPRAGMAPVAS |
| | | KRKAMQATTTRLVNRIWGEFYSNYSPEDPLQATAAENGEDEVEEEGGNGE |
| | | EEVEEEGENGLTEDTVPEPVEVQKPHTPKKIRGSSGKREIKWDGESLGKTSA |
| | | GEPLYQQALVGGEMVAVGGAVTLEVDDPDEMPAIYFVEYMFESTDHCKM |
| | | LHGRFLQRGSMTVLGNAANERELFLTNECMTTQLKDIKGVASFEIRSRPWG |
| | | HQYRKKNITADKLDWARALERKVKDLPTEYYCKSLYSPERGGFFSLPLSDI |
| | | GRSSGFCTSCKIREDEEKRSTIKLNVSKTGFFINGIEYSVEDFVYVNPDSIGGL |
| | | KEGSKTSFKSGRNIGLRAYVVCQLLEIVPKESRKADLGSFDVKVRRFYRPED |
| | | VSAEKAYASDIQELYFSQDTVVLPPGALEGKCEVRKKSDMPLSREYPISDHI |
| | | FFCDLFFDTSKGSLKQLPANMKPKFSTIKDDTLLRKKKGKGVESEIESEIVKP |
| | | VEPPKEIRLATLDIFAGCGGLSHGLKKAGVSDAKWAIEYEEPAGQAFKQNH |
| | | PESTVFVDNCNVILRAIMEKGGDQDDCVSTTEANELAAKLTEEQKSTLPLP |
| | | GQVDFINGGPPCQGFSGMNRFNQSSWSKVQCEMILAFLSFADYFRPRYFLL |
| | | ENVRTFVSFNKGQTFQLTLASLLEMGYQVRFGILEAGAYGVSQSRKRAFIW |
| | | AAAPEEVLPEWPEPMHVFGVPKLKISLSQGLHYAAVRSTALGAPFRPITVRD |
| | | TIGDLPSVENGDSRTNKEYKEVAVSWFQKEIRGNTIALTDHICKAMNELNLI |
| | | RCKLIPTRPGADWHDLPKRKVTLSDGRVEEMIPFCLPNTAERHNGWKGLY |
| | | GRLDWQGNFPTSVTDPQPMGKVGMCFHPEQHRILTVRECARSQGFPDSYEF |
| | | AGNINHKHRQIGNAVPPPLAFALGRKLKEALHLKKSPQHQP |
| 51 | Ascobolus | MELTPELSGVSTDLGGGGSIFAHWRMKEESPAPTEILDDLNVLEWEKTTRD |
| | Masc2 | YSKEDLRIADQLFSIEDEHQSLPFETADAEDGTPTEEEEEKELPMRTLDNFVL |
| | | YDASDLELAALDLIGTELNIHAVGTVGPIYTEGEEDEQEDEDEDVSPPVRTG |
| | | TQATSASVTQMTVELYIRNIVQYEFCFNDDGTVETWIQTTNAHYKLLQPAK |
| | | CYTSLYRPVNDCLNVITAIITLAPESTTMSLKDLLKVMDDKAQAVSYEEVE |
| | | RMSEFIVQHLDQWMETAPKKKSKLIEKSKVYIDLNNLAGIDMVSGVRPPPV |
| | | RRVTGRSSAPKKRIVRNMNDAVLLHQNETTVTNWIHQLSAGMFGRALNVL |
| | | GAETADVENLTCDPASAKFVVPQRRLHKRLKWETRGHIPVSEEEYKHIYQG |
| | | KKYAKFFEAVRAVDESKLTIKLGDLVYVLDQDPKVTQTQFATAGREGRKK |
| | | GAEKEKIQVRFGRVLSIRQPDSNSKDAQNVFIHVQWLVLGCDTILQEMASR |
| | | RELFLTDSCDTVFADVIYGVAKLTPLGAKDIPTVEFHESMATMMGENEFFV |
| | | RFKYNYQDGSFTDLKDVDAEQIGTLQPRVNTHRNPGYCSNCRIKYDNERTG |
| | | DKWIYENDTEGEPRLFRSSKGWCIYAQEFVYLQPVEKQPGTTFRVGYISEIN |
| | | KSSVIVELLARVDDDDKSGHISYSDPRHLYFTGTDIKVTFDKIIRKCFVFHDS |
| | | GDQKAKAPLMYGTLQRDLYYYRYEKRKGKAELVPVREIRSIHEQTLNDWE |
| | | SRTQIERHGAVSGKKLKGLDIFAGCGGLTLGLDLSGAVDTKWDIEFAPSAA |
| | | NTLALNFPDAQVFNQCANVLLSRAIQSEDEGSLDIEYDLQGRVLPDLPKKG |
| | | EVDFIYGGPPCQGFSGVNRYKKGNDIKNSLVATFLSYVDHYKPRFVLLENV |
| | | KGLITTKLGNSKNAEGKWEGGISNGVVKFIYRTLISMNYQCRIGLVQSGEY |
| | | GVPQSRPRVIFLAARMGERLPDLPEPMHAFEVLDSQYALPHIKRYHTTQNG |
| | | VAPLPRITIGEAVSDLPKFQYANPGVWPRHDPYSSAKAQPSDKTIEKFSVSK |
| | | ATSFVGYLLQPYHSRPQSEFQRRLRTKLVPSDEPAEKTSLLTTKLVTAHVTR |
| | | LFNKETTQRIVCVPMWPGADHRSLPKEMRPWCLVDPNSQAEKHRFWPGLF |
| | | GRLGMEDFFSTALTDVQPCGKQGKVLHPTQRRVYTVRELARAQGFPDWFA |
| | | FTDGDADSGLGGVKKWHRNIGNAVPVPLGEQIGRCIGYSVWWKDDMIAQ |
| | | LREDGADEDEEMIDGNDQWVEELNTQMAADMPGLPLLVTHLLNLCVYRR |
| | | LYGPNAKEFLPARVYDKKLEGGRRRLVWAML |
| 52 | Neurospora | MDSPDRSHGGMFIDVPAETMGFQEDYLDMFASVLSQGLAKEGDYAHHQPL |
| | Dim2 | PAGKEECLEPIAVATTITPSPDDPQLQLQLELEQQFQTESGLNGVDPAPAPES |
| | | EDEADLPDGFSDESPDDDFVVQRSKHITVDLPVSTLINPRSTFQRIDENDNLV |
| | | PPPQSTPERVAVEDLLKAAKAAGKNKEDYIEFELHDFNFYVNYAYHPQEM |
| | | RPIQLVATKVLHDKYYFDGVLKYGNTKHYVTGMQVLELPVGNYGASLHS |
| | | VKGQIWVRSKHNAKKEIYYLLKKPAFEYQRYYQPFLWIADLGKHVVDYCT |
| | | RMVERKREVTLGCFKSDFIQWASKAHGKSKAFQNWRAQHPSDDFRTSVAA |
| | | NIGYIWKEINGVAGAKRAAGDQLFRELMIVKPGQYFRQEVPPGPVVTEGDR |
| | | TVAATIVTPYIKECFGHMILGKVLRLAGEDAEKEKEVKLAKRLKIENKNAT |
| | | KADTKDDMKNDTATESLPTPLRSLPVQVLEATPIESDIVSIVSSDLPPSENNP |
| | | PPLTNGSVKPKAKANPKPKPSTQPLHAAHVKYLSQELVNKIKVGDVISTPR |
| | | DDSSNTDTKWKPTDTDDHRWFGLVQRVHTAKTKSSGRGLNSKSFDVIWFY |
| | | RPEDTPCCAMKYKWRNELFLSNHCTCQEGHHARVKGNEVLAVHPVDWFG |
| | | TPESNKGEFFVRQLYESEQRRWITLQKDHLTCYHNQPPKPPTAPYKPGDTV |
| | | LATLSPSDKFSDPYEVVEYFTQGEKETAFVRLRKLLRRRKVDRQDAPANEL |
| | | VYTEDLVDVRAERIVGKCIMRCFRPDERVPSPYDRGGTGNMFFITHRQDHG |
| | | RCVPLDTLPPTLRQGFNPLGNLGKPKLRGMDLYCGGGNFGRGLEEGGVVE |
| | | MRWANDIWDKAIHTYMANTPDPNKTNPFLGSVDDLLRLALEGKFSDNVPR |
| | | PGEVDFIAAGSPCPGFSLLTQDKKVLNQVKNQSLVASFASFVDFYRPKYGV |
| | | LENVSGIVQTFVNRKQDVLSQLFCALVGMGYQAQLILGDAWAHGAPQSRE |
| | | RVFLYFAAPGLPLPDPPLPSHSHYRVKNRNIGFLCNGESYVQRSFIPTAFKFV |
| | | SAGEGTADLPKIGDGKPDACVRFPDHRLASGITPYIRAQYACIPTHPYGMNF |
| | | IKAWNNGNGVMSKSDRDLFPSEGKTRTSDASVGWKRLNPKTLFPTVTTTS |
| | | NPSDARMGPGLHWDEDRPYTVQEMRRAQGYLDEEVLVGRTTDQWKLVG |
| | | NSVSRHMALAIGLKFREAWLGTLYDESAVVATATATATTAAAVGVTVPV |
| | | MEEPGIGTTESSRPSRSPVHTAVDLDDSKSERSRSTTPATVLSTSSAAGDGSA |
| | | NAAGLEDDDNDDMEMMEVTRKRSSPAVDEEGMRPSKVQKVEVTVASPAS |
| | | RRSSRQASRNPTASPSSKASKATTHEAPAPEELESDAESYSETYDKEGFDGD |
| | | YHSGHEDQYSEEDEEEEYAEPETMTVNGMTIVKL |
| 53 | Drosophila | MVFRVLELFSGIGGMHYAFNYAQLDGQIVAALDVNTVANAVYAHNYGSN |
| | dDnmt2 | LVKTRNIQSLSVKEVTKLQANMLLMSPPCQPHTRQGLQRDTEDKRSDALTH |
| | | LCGLIPECQELEYILMENVKGFESSQARNQFIESLERSGFHWREFILTPTQFN |
| | | VPNTRYRYYCIARKGADFPFAGGKIWEEMPGAIAQNQGLSQIAEIVEENVSP |
| | | DFLVPDDVLTKRVLVMDIIHPAQSRSMCFTKGYTHYTEGTGSAYTPLSEDE |
| | | SHRIFELVKEIDTSNQDASKSEKILQQRLDLLHQVRLRYFTPREVARLMSFPE |
| | | NFEFPPETTNRQKYRLLGNSINVKVVGELIKLLTIK |
| 54 | S. pombe | MLSTKRLRVLELYSGIGGMHYALNLANIPADIVCAIDINPQANEIYNLNHGK |
| | Pmt1 | LAKHMDISTLTAKDFDAFDCKLWTMSPSCQPFTRIGNRKDILDPRSQAFLNI |
| | | LNVLPHVNNLPEYILIENVQGFEESKAAEECRKVLRNCGYNLIEGILSPNQFN |
| | | IPNSRSRWYGLARLNFKGEWSIDDVFQFSEVAQKEGEVKRIRDYLEIERDW |
| | | SSYMVLESVLNKWGHQFDIVKPDSSSCCCFTRGYTHLVQGAGSILQMSDHE |
| | | NTHEQFERNRMALQLRYFTAREVARLMGFPESLEWSKSNVTEKCMYRLLG |
| | | NSINVKVVSYLISLLLEPLNF |
| 55 | Arabidopsis | MVMSHIFLISQIQEVEHGDSDDVNWNTDDDELAIDNFQFSPSPVHISATSPNS |
| | DRM1 | IQNRISDETVASFVEMGFSTQMIARAIEETAGANMEPMMILETLFNYSASTE |
| | | ASSSKSKVINHFIAMGFPEEHVIKAMQEHGDEDVGEITNALLTYAEVDKLRE |
| | | SEDMNININDDDDDNLYSLSSDDEEDELNNSSNEDRILQALIKMGYLREDA |
| | | AIAIERCGEDASMEEVVDFICAAQMARQFDEIYAEPDKKELMNNNKKRRTY |
| | | TETPRKPNTDQLISLPKEMIGFGVPNHPGLMMHRPVPIPDIARGPPFFYYENV |
| | | AMTPKGVWAKISSHLYDIVPEFVDSKHFCAAARKRGYIHNLPIQNRFQIQPP |
| | | QHNTIQEAFPLTKRWWPSWDGRTKLNCLLTCIASSRLTEKIREALERYDGET |
| | | PLDVQKWVMYECKKWNLVWVGKNKLAPLDADEMEKLLGFPRDHTRGGG |
| | | ISTTDRYKSLGNSFQVDTVAYHLSVLKPLFPNGINVLSLFTGIGGGEVALHR |
| | | LQIKMNVVVSVEISDANRNILRSFWEQTNQKGILREFKDVQKLDDNTIERL |
| | | MDEYGGFDLVIGGSPCNNLAGGNRHHRVGLGGEHSSLFFDYCRILEAVRRK |
| | | ARHMRR |
| 56 | Arabadopsis | MVIWNNDDDDFLEIDNFQSSPRSSPIHAMQCRVENLAGVAVTTSSLSSPTET |
| | DRM2 | TDLVQMGFSDEVFATLFDMGFPVEMISRAIKETGPNVETSVIIDTISKYSSDC |
| | | EAGSSKSKAIDHFLAMGFDEEKVVKAIQEHGEDNMEAIANALLSCPEAKKL |
| | | PAAVEEEDGIDWSSSDDDTNYTDMLNSDDEKDPNSNENGSKIRSLVKMGFS |
| | | ELEASLAVERCGENVDIAELTDFLCAAQMAREFSEFYTEHEEQKPRHNIKK |
| | | RRFESKGEPRSSVDDEPIRLPNPMIGFGVPNEPGLITHRSLPELARGPPFFYYE |
| | | NVALTPKGVWETISRHLFEIPPEFVDSKYFCVAARKRGYIHNLPINNRFQIQP |
| | | PPKYTIHDAFPLSKRWWPEWDKRTKLNCILTCTGSAQLTNRIRVALEPYNE |
| | | EPEPPKHVQRYVIDQCKKWNLVWVGKNKAAPLEPDEMESILGFPKNHTRG |
| | | GGMSRTERFKSLGNSFQVDTVAYHLSVLKPIFPHGINVLSLFTGIGGGEVAL |
| | | HRLQIKMKLVVSVEISKVNRNILKDFWEQTNQTGELIEFSDIQHLTNDTIEGL |
| | | MEKYGGFDLVIGGSPCNNLAGGNRVSRVGLEGDQSSLFFEYCRILEVVRAR |
| | | MRGS |
| 57 | Arabadopsis | MAARNKQKKRAEPESDLCFAGKPMSVVESTIRWPHRYQSKKTKLQAPTKK |
| | CMT1 | PANKGGKKEDEEIIKQAKCHFDKALVDGVLINLNDDVYVTGLPGKLKFIAK |
| | | VIELFEADDGVPYCRFRWYYRPEDTLIERFSHLVQPKRVFLSNDENDNPLTC |
| | | IWSKVNIAKVPLPKITSRIEQRVIPPCDYYYDMKYEVPYLNFTSADDGSDAS |
| | | SSLSSDSALNCFENLHKDEKFLLDLYSGCGAMSTGFCMGASISGVKLITKWS |
| | | VDINKFACDSLKLNHPETEVRNEAAEDFLALLKEWKRLCEKFSLVSSTEPVE |
| | | SISELEDEEVEENDDIDEASTGAELEPGEFEVEKFLGIMFGDPQGTGEKTLQL |
| | | MVRWKGYNSSYDTWEPYSGLGNCKEKLKEYVIDGFKSHLLPLPGTVYTVC |
| | | GGPPCQGISGYNRYRNNEAPLEDQKNQQLLVFLDIIDFLKPNYVLMENVVD |
| | | LLRFSKGFLARHAVASFVAMNYQTRLGMMAAGSYGLPQLRNRVFLWAAQ |
| | | PSEKLPPYPLPTHEVAKKENTPKEFKDLQVGRIQMEFLKLDNALTLADAISD |
| | | LPPVTNYVANDVMDYNDAAPKTEFENFISLKRSETLLPAFGGDPTRRLFDH |
| | | QPLVLGDDDLERVSYIPKQKGANYRDMPGVLVHNNKAEINPRFRAKLKSG |
| | | KNVVPAYAISFIKGKSKKPFGRLWGDEIVNTVVTRAEPHNQCVIHPMQNRV |
| | | LSVRENARLQGFPDCYKLCGTIKEKYIQVGNAVAVPVGVALGYAFGMASQ |
| | | GLTDDEPVIKLPFKYPECMQAKDQI |
| 58 | Arabadopsis | MLSPAKCESEEAQAPLDLHSSSRSEPECLSLVLWCPNPEEAAPSSTRELIKLP |
| | CMT2 | DNGEMSLRRSTTLNCNSPEENGGEGRVSQRKSSRGKSQPLLMLTNGCQLRR |
| | | SPRFRALHANFDNVCSVPVTKGGVSQRKFSRGKSQPLLTLTNGCQLRRSPR |
| | | FRAVDGNFDSVCSVPVTGKFGSRKRKSNSALDKKESSDSEGLTFKDIAVIAK |
| | | SLEMEIISECQYKNNVAEGRSRLQDPAKRKVDSDTLLYSSINSSKQSLGSNK |
| | | RMRRSQRFMKGTENEGEENLGKSKGKGMSLASCSFRRSTRLSGTVETGNT |
| | | ETLNRRKDCGPALCGAEQVRGTERLVQISKKDHCCEAMKKCEGDGLVSSK |
| | | QELLVFPSGCIKKTVNGCRDRTLGKPRSSGLNTDDIHTSSLKISKNDTSNGLT |
| | | MTTALVEQDAMESLLQGKTSACGAADKGKTREMHVNSTVIYLSDSDEPSSI |
| | | EYLNGDNLTQVESGSALSSGGNEGIVSLDLNNPTKSTKRKGKRVTRTAVQE |
| | | QNKRSICFFIGEPLSCEEAQERWRWRYELKERKSKSRGQQSEDDEDKIVAN |
| | | VECHYSQAKVDGHTFSLGDFAYIKGEEEETHVGQIVEFFKTTDGESYFRVQ |
| | | WFYRATDTIMERQATNHDKRRLFYSTVMNDNPVDCLISKVTVLQVSPRVG |
| | | LKPNSIKSDYYFDMEYCVEYSTFQTLRNPKTSENKLECCADVVPTESTESIL |
| | | KKKSFSGELPVLDLYSGCGGMSTGLSLGAKISGVDVVTKWAVDQNTAACK |
| | | SLKLNHPNTQVRNDAAGDFLQLLKEWDKLCKRYVFNNDQRTDTLRSVNST |
| | | KETSGSSSSSDDDSDSEEYEVEKLVDICFGDHDKTGKNGLKFKVHWKGYRS |
| | | DEDTWELAEELSNCQDAIREFVTSGFKSKILPLPGRVGVICGGPPCQGISGYN |
| | | RHRNVDSPLNDERNQQIIVFMDIVEYLKPSYVLMENVVDILRMDKGSLGRY |
| | | ALSRLVNMRYQARLGIMTAGCYGLSQFRSRVFMWGAVPNKNLPPFPLPTH |
| | | DVIVRYGLPLEFERNVVAYAEGQPRKLEKALVLKDAISDLPHVSNDEDREK |
| | | LPYESLPKTDFQRYIRSTKRDLTGSAIDNCNKRTMLLHDHRPFHINEDDYAR |
| | | VCQIPKRKGANFRDLPGLIVRNNTVCRDPSMEPVILPSGKPLVPGYVFTFQQ |
| | | GKSKRPFARLWWDETVPTVLTVPTCHSQALLHPEQDRVLTIRESARLQGFP |
| | | DYFQFCGTIKERYCQIGNAVAVSVSRALGYSLGMAFRGLARDEHLIKLPQN |
| | | FSHSTYPQLQETIPH |
| 59 | Arabadopsis | MAPKRKRPATKDDTTKSIPKPKKRAPKRAKTVKEEPVTVVEEGEKHVARFL |
| | CMT3 | DEPIPESEAKSTWPDRYKPIEVQPPKASSRKKTKDDEKVEIIRARCHYRRAIV |
| | | DERQIYELNDDAYVQSGEGKDPFICKIIEMFEGANGKLYFTARWFYRPSDT |
| | | VMKEFEILIKKKRVFFSEIQDTNELGLLEKKLNILMIPLNENTKETIPATENCD |
| | | FFCDMNYFLPYDTFEAIQQETMMAISESSTISSDTDIREGAAAISEIGECSQET |
| | | EGHKKATLLDLYSGCGAMSTGLCMGAQLSGLNLVTKWAVDMNAHACKS |
| | | LQHNHPETNVRNMTAEDFLFLLKEWEKLCIHFSLRNSPNSEEYANLHGLNN |
| | | VEDNEDVSEESENEDDGEVFTVDKIVGISFGVPKKLLKRGLYLKVRWLNYD |
| | | DSHDTWEPIEGLSNCRGKIEEFVKLGYKSGILPLPGGVDVVCGGPPCQGISG |
| | | HNRFRNLLDPLEDQKNKQLLVYMNIVEYLKPKFVLMENVVDMLKMAKGY |
| | | LARFAVGRLLQMNYQVRNGMMAAGAYGLAQFRLRFFLWGALPSEIIPQFP |
| | | LPTHDLVHRGNIVKEFQGNIVAYDEGHTVKLADKLLLKDVISDLPAVANSE |
| | | KRDEITYDKDPTTPFQKFIRLRKDEASGSQSKSKSKKHVLYDHHPLNLNIND |
| | | YERVCQVPKRKGANFRDFPGVIVGPGNVVKLEEGKERVKLESGKTLVPDY |
| | | ALTYVDGKSCKPFGRLWWDEIVPTVVTRAEPHNQVIIHPEQNRVLSIRENA |
| | | RLQGFPDDYKLFGPPKQKYIQVGNAVAVPVAKALGYALGTAFQGLAVGK |
| | | DPLLTLPEGFAFMKPTLPSELA |
| 60 | Neurospora | MAEQNPFVIDDEDDVIQIHDEEEVEEEVAEVIDITEDDIEPSELDRAFGSRPK |
| | Rid | EETLPSLLLRDQGFIVRPGMTVELKAPIGRFAISFVRVNSIVKVRQAHVNNV |
| | | TIRGHGFTRAKEMNGMLPKQLNECCLVASIDTRDPRP |
| 61 | E. coli | MNNNDLVAKLWKLCDNLRDGGVSYQNYVNELASLLFLKMCKETGQEAE |
| | strain 12 | YLPEGYRWDDLKSRIGQEQLQFYRKMLVHLGEDDKKLVQAVFHNVSTTIT |
| | hsdM | EPKQITALVSNMDSLDWYNGAHGKSRDDFGDMYEGLLQKNANETKSGAG |
| | | QYFTPRPLIKTIIHLLKPQPREVVQDPAAGTAGFLIEADRYVKSQTNDLDDL |
| | | DGDTQDFQIHRAFIGLELVPGTRRLALMNCLLHDIEGNLDHGGAIRLGNTL |
| | | GSDGENLPKAHIVATNPPFGSAAGTNITRTFVHPTSNKQLCFMQHIIETLHPG |
| | | GRAAVVVPDNVLFEGGKGTDIRRDLMDKCHLHTILRLPTGIFYAQGVKTNV |
| | | LFFTKGTVANPNQDKNCTDDVWVYDLRTNMPSFGKRTPFTDEHLQPFERV |
| | | YGEDPHGLSPRTEGEWSFNAEETEVADSEENKNTDQHLATSRWRKFSREWI |
| | | RTAKSDSLDISWLKDKDSIDADSLPEPDVLAAEAMGELVQALSELDALMRE |
| | | LGASDEADLQRQLLEEAFGGVKE |
| 62 | E. coli | MSAGKLPEGWVIAPVSTVTTLIRGVTYKKEQAINYLKDDYLPLIRANNIQN |
| | strain 12 | GKFDTTDLVFVPKNLVKESQKISPEDIVIAMSSGSKSVVGKSAHQHLPFECS |
| | hsdS | FGAFCGVLRPEKLIFSGFIAHFTKSSLYRNKISSLSAGANINNIKPASFDLINIPI |
| | | PPLAEQKIIAEKLDTLLAQVDSTKARFEQIPQILKRFRQAVLGGAVNGKLTE |
| | | KWRNFEPQHSVFKKLNFESILTELRNGLSSKPNESGVGHPILRISSVRAGHV |
| | | DQNDIRFLECSESELNRHKLQDGDLLFTRYNGSLEFVGVCGLLKKLQHQNL |
| | | LYPDKLIRARLTKDALPEYIEIFFSSPSARNAMMNCVKTTSGQKGISGKDIKS |
| | | QVVLLPPVKEQAEIVRRVEQLFAYADTIEKQVNNALARVNNLTQSILAKAF |
| | | RGELTAQWRAENPDLISGENSAAALLEKIKAERAASGGKKASRKKS |
| 63 | T. | MGLPPLLSLPSNSAPRSLGRVETPPEVVDFMVSLAEAPRGGRVLEPACAHGP |
| | aquaticus | FLRAFREAHGTAYRFVGVEIDPKALDLPPWAEGILADFLLWEPGEAFDLILG |
| | M TaqI | NPPYGIVGEASKYPIHVFKAVKDLYKKAFSTWKGKYNLYGAFLEKAVRLL |
| | | KPGGVLVFVVPATWLVLEDFALLREFLAREGKTSVYYLGEVFPQKKVSAV |
| | | VIRFQKSGKGLSLWDTQESESGFTPILWAEYPHWEGEIIRFETEETRKLEISG |
| | | MPLGDLFHIRFAARSPEFKKHPAVRKEPGPGLVPVLTGRNLKPGWVDYEKN |
| | | HSGLWMPKERAKELRDFYATPHLVVAHTKGTRVVAAWDERAYPWREEFH |
| | | LLPKEGVRLDPSSLVQWLNSEAMQKHVRTLYRDFVPHLTLRMLERLPVRR |
| | | EYGFHTSPESARNF |
| 64 | E. coli | MKKNRAFLKWAGGKYPLLDDIKRHLPKGECLVEPFVGAGSVFLNTDFSRYI |
| | M EcoDam | LADINSDLISLYNIVKMRTDEYVQAARELFVPETNCAEVYYQFREEFNKSQ |
| | | DPFRRAVLFLYLNRYGYNGLCRYNLRGEFNVPFGRYKKPYFPEAELYHFAE |
| | | KAQNAFFYCESYADSMARADDASVVYCDPPYAPLSATANFTAYHTNSFTL |
| | | EQQAHLAEIAEGLVERHIPVLISNHDTMLTREWYQRAKLHVVKVRRSISSN |
| | | GGTRKKVDELLALYKPGVVSPAKK |
| 65 | C. | MKFGPETIIHGDCIEQMNALPEKSVDLIFADPPYNLQLGGDLLRPDNSKVDA |
| | crescentus | VDDHWDQFESFAAYDKFTREWLKAARRVLKDDGAIWVIGSYHNIFRVGV |
| | M CcrMI | AVQDLGFWILNDIVWRKSNPMPNFKGTRFANAHETLIWASKSQNAKRYTF |
| | | NYDALKMANDEVQMRSDWTIPLCTGEERIKGADGQKAHPTQKPEALLYRV |
| | | ILSTTKPGDVILDPFFGVGTTGAAAKRLGRKFIGIEREAEYLEHAKARIAKVV |
| | | PIAPEDLDVMGSKRAEPRVPFGTIVEAGLLSPGDTLYCSKGTHVAKVRPDGS |
| | | ITVGDLSGSIHKIGALVQSAPACNGWTYWHFKTDAGLAPIDVLRAQVRAG |
| | | MIN |
| 66 | C. difficile | MDDISQDNFLLSKEYENSLDVDTKKASGIYYTPKIIVDYIVKKTLKNHDIIKN |
| | CamA | PYPRILDISCGCGNFLLEVYDILYDLFEENIYELKKKYDENYWTVDNIHRHIL |
| | | NYCIYGADIDEKAISILKDSLTNKKVVNDLDESDIKINLFCCDSLKKKWRYK |
| | | FDYIVGNPPYIGHKKLEKKYKKFLLEKYSEVYKDKADLYFCFYKKIIDILKQ |
| | | GGIGSVITPRYFLESLSGKDLREYIKSNVNVQEIVDFLGANIFKNIGVSSCILT |
| | | FDKKKTKETYIDVFKIKNEDICINKFETLEELLKSSKFEHFNINQRLLSDEWIL |
| | | VNKDDETFYNKIQEKCKYSLEDIAISFQGIITGCDKAFILSKDDVKLNLVDD |
| | | KFLKCWIKSKNINKYIVDKSEYRLIYSNDIDNENTNKRILDEIIGLYKTKLEN |
| | | RRECKSGIRKWYELQWGREKLFFERKKIMYPYKSNENRFAIDYDNNFSSAD |
| | | VYSFFIKEEYLDKFSYEYLVGILNSSVYDKYFKITAKKMSKNIYDYYPNKV |
| | | MKIRIFRDNNYEEIENLSKQIISILLNKSIDKGKVEKLQIKMDNLIMDSLGI |
| 67 | ZIM3 | MNNSQGRVTFEDVTVNFTQGEWQRLNPEQRNLYRDVMLENYSNLVSVGQ |
| | | GETTKPDVILRLEQGKEPWLEEEEVLGSGRAEKNGDIGGQIWKPKDVKESL |
| 68 | ZNF436 | MAATLLMAGSQAPVTFEDMAMYLTREEWRPLDAAQRDLYRDVMQENYG |
| | | NVVSLDFEIRSENEVNPKQEISEDVQFGTTSERPAENAEENPESEEGFESGDR |
| | | SERQW |
| 69 | ZNF257 | MLENYRNLVFLGIAVSKPDLITCLEQGKEPCNMKRHEMVAKPPVMCSHIAE |
| | | DLCPERDIKYFFQKVILRRYDKCEHENLQLRKGCKSVDECKVCK |
| 70 | ZNF675 | MGLLTFRDVAIEFSLEEWQCLDTAQRNLYKNVILENYRNLVFLGIAVSKQD |
| | | LITCLEQEKEPLTVKRHEMVNEPPVMCSHFAQEFWPEQNIKDSF |
| 71 | ZNF490 | MLQMQNSEHHGQSIKTQTDSISLEDVAVNFTLEEWALLDPGQRNIYRDVM |
| | | RATFKNLACIGEKWKDQDIEDEHKNQGRNLRSPMVEALCENKEDCPCGKS |
| | | TSQIPDLNTNLETPTG |
| 72 | ZNF320 | MALSQGLLTFRDVAIEFSQEEWKCLDPAQRTLYRDVMLENYRNLVSLDISS |
| | | KCMMNTLSSTGQGNTEVIHTGTLQRQASYHIGAFCSQEIEKDIHDFVFQ |
| 73 | ZNF331 | MAQGLVTFADVAIDFSQEEWACLNSAQRDLYWDVMLENYSNLVSLDLES |
| | | AYENKSLPTKKNIHEIRASKRNSDRRSKSLGRNWICEGTLERPQRSRGR |
| 74 | ZNF816 | MLREEATKKSKEKEPGMALPQGRLTFRDVAIEFSLEEWKCLNPAQRALYR |
| | | AVMLENYRNLEFVDSSLKSMMEFSSTRHSITGEVIHTGTLQRHKSHHIGDFC |
| | | FPEMKKDIHHFEFQWQ |
| 75 | ZNF680 | MPGPPGSLEMGPLTFRDVAIEFSLEEWQCLDTAQRNLYRKVMFENYRNLVF |
| | | LGIAVSKPHLITCLEQGKEPWNRKRQEMVAKPPVIYSHFTEDLWPEHSIKDS |
| | | F |
| 76 | ZNF41 | MSPPWSPALAAEGRGSSCEASVSFEDVTVDFSKEEWQHLDPAQRRLYWDV |
| | | TLENYSHLLSVGYQIPKSEAAFKLEQGEGPWMLEGEAPHQSCSGEAIGKMQ |
| | | QQGIPGGIFFHC |
| 77 | ZNF189 | MASPSPPPESKEEWDYLDPAQRSLYKDVMMENYGNLVSLDVLNRDKDEEP |
| | | TVKQEIEEIEEEVEPQGVIVTRIKSEIDQDPMGRETFELVGRLDKQRGIFLWEI |
| | | PRESL |
| 78 | ZNF528 | MALTQGPLKFMDVAIEFSQEEWKCLDPAQRTLYRDVMLENYRNLVSLGIC |
| | | LPDLSVTSMLEQKRDPWTLQSEEKIANDPDGRECIKGVNTERSSKLGSN |
| 79 | ZNF543 | MAASAQVSVTFEDVAVTFTQEEWGQLDAAQRTLYQEVMLETCGLLMSLG |
| | | CPLFKPELIYQLDHRQELWMATKDLSQSSYPGDNTKPKTTEPTFSHLALPE |
| 80 | ZNF554 | MFSQEERMAAGYLPRWSQELVTFEDVSMDFSQEEWELLEPAQKNLYREV |
| | | MLENYRNVVSLEALKNQCTDVGIKEGPLSPAQTSQVTSLSSWTGYLLFQPV |
| | | ASSHLEQREALWIEEKGTPQASCSDWMTVLRNQDSTYKKVALQE |
| 81 | ZNF140 | MSQGSVTFRDVAIDFSQEEWKWLQPAQRDLYRCVMLENYGHLVSLGLSIS |
| | | KPDVVSLLEQGKEPWLGKREVKRDLFSVSESSGEIKDFSPKNVIYDD |
| 82 | ZNF610 | MEEAQKRKAKESGMALPQGRLTFMDVAIEFSQEEWKSLDPGQRALYRDV |
| | | MLENYRNLVFLGRSCVLGSNAENKPIKNQLGLTLESHLSELQLFQAGRKIY |
| | | RSNQVEKFTNHR |
| 83 | ZNF264 | MAAAVLTDRAQVSVTFDDVAVTFTKEEWGQLDLAQRTLYQEVMLENCGL |
| | | LVSLGCPVPKAELICHLEHGQEPWTRKEDLSQDTCPGDKGKPKTTEPTTCEP |
| | | ALSE |
| 84 | ZNF350 | MIQAQESITLEDVAVDFTWEEWQLLGAAQKDLYRDVMLENYSNLVAVGY |
| | | QASKPDALFKLEQGEQLWTIEDGIHSGACSDIWKVDHVLERLQSESLVNR |
| 85 | ZNF8 | MEGVAGVMSVGPPAARLQEPVTFRDVAVDFTQEEWGQLDPTQRILYRDV |
| | | MLETFGHLLSIGPELPKPEVISQLEQGTELWVAERGTTQGCHPAWEPRSESQ |
| | | ASRKEEGLPEE |
| 86 | ZNF582 | MSLGSELFRDVAIVFSQEEWQWLAPAQRDLYRDVMLETYSNLVSLGLAVS |
| | | KPDVISFLEQGKEPWMVERVVSGGLCPVLESRYDTKELFPKQHVYEV |
| 87 | ZNF30 | MAHKYVGLQYHGSVTFEDVAIAFSQQEWESLDSSQRGLYRDVMLENYRN |
| | | LVSMAGHSRSKPHVIALLEQWKEPEVTVRKDGRRWCTDLQLEDDTIGCKE |
| | | MPTSEN |
| 88 | ZNF324 | MAFEDVAVYFSQEEWGLLDTAQRALYRRVMLDNFALVASLGLSTSRPRVV |
| | | IQLERGEEPWVPSGTDTTLSRTTYRRRNPGSWSLTEDRDVSG |
| 89 | ZNF98 | MLENYRNLVFVGIAASKPDLITCLEQGKEPWNVKRHEMVTEPPVVYSYFA |
| | | QDLWPKQGKKNYFQKVILRTYKKCGRENLQLRKYCKSMDECKVHKECYN |
| | | GLNQC |
| 90 | ZNF669 | MHFRRPDPCREPLASPIQDSVAFEDVAVNFTQEEWALLDSSQKNLYREVMQ |
| | | ETCRNLASVGSQWKDQNIEDHFEKPGKDIRNHIVQRLCESKEDGQYGEVVS |
| | | QIPNLDLNENISTGLKPCECSICGK |
| 91 | ZNF677 | MALSQGLFTFKDVAIEFSQEEWECLDPAQRALYRDVMLENYRNLLSLDED |
| | | NIPPEDDISVGFTSKGLSPKENNKEELYHLVILERKESHGINNFDLKEVWEN |
| | | MPKFDSLW |
| 92 | ZNF596 | MTFEDIIVDFTQEEWALLDTSQRKLFQDVMLENISHLVSIGKQLCKSVVLSQ |
| | | LEQVEKLSTQRISLLQGREVGIKHQEIPFIHHIYQKGTSTISTMRS |
| 93 | ZNF214 | MAVTFEDVTIIFTWEEWKFLDSSQKRLYREVMWENYTNVMSVENWNESY |
| | | KSQEEKFRYLEYENFSYWQGWWNAGAQMYENQNYGETVQGTDSKDLTQ |
| | | QDRSQC |
| 94 | ZNF37A | MITSQGSVSFRDVTVGFTQEEWQHLDPAQRTLYRDVMLENYSHLVSVGYC |
| | | IPKPEVILKLEKGEEPWILEEKFPSQSHLELINTSRNYSIMKFNEFNKG |
| 95 | ZNF34 | MFEDVAVYLSREEWGRLGPAQRGLYRDVMLETYGNLVSLGVGPAGPKPG |
| | | VISQLERGDEPWVLDVQGTSGKEHLRVNSPALGTRTEYKELTSQETFGEED |
| | | PQGSEPVEACDHIS |
| 96 | ZNF250 | METYGNVVSLGLPGSKPDIISQLERGEDPWVLDRKGAKKSQGLWSDYSDN |
| | | LKYDHTTACTQQDSLSCPWECETKGESQNTDLSPKPLISEQTVILGKTPLGRI |
| | | DQENNETKQ |
| 97 | ZNF547 | MAEMNPAQGHVVFEDVAIYFSQEEWGHLDEAQRLLYRDVMLENLALLSSL |
| | | GCCHGAEDEEAPLEPGVSVGVSQVMAPKPCLSTQNTQPCETCSSLLKDILRL |
| 98 | ZNF273 | MLDNYRNLVFLGIAVSKPDLITCLEQGKEPCNMKRHAMVAKPPVVCSHFA |
| | | QDLWPKQGLKDS |
| 99 | ZNF354A | MAAGQREARPQVSLTFEDVAVLFTRDEWRKLAPSQRNLYRDVMLENYRN |
| | | LVSLGLPFTKPKVISLLQQGEDPWEVEKDGSGVSSLGSKSSHKTTKSTQTQD |
| | | SSFQ |
| 100 | ZFP82 | MALRSVMFSDVSIDFSPEEWEYLDLEQKDLYRDVMLENYSNLVSLGCFISK |
| | | PDVISSLEQGKEPWKVVRKGRRQYPDLETKYETKKLSLENDIYEIN |
| 101 | ZNF224 | MTTFKEAMTFKDVAVVFTEEELGLLDLAQRKLYRDVMLENFRNLLSVGHQ |
| | | AFHRDTFHFLREEKIWMMKTAIQREGNSGDKIQTEMETVSEAGTHQEW |
| 102 | ZNF33A | MFQVEQKSQESVSFKDVTVGFTQEEWQHLDPSQRALYRDVMLENYSNLVS |
| | | VGYCVHKPEVIFRLQQGEEPWKQEEEFPSQSFPEVWTADHLKERSQENQSK |
| | | HL |
| 103 | ZNF45 | MTKSKEAVTFKDVAVVFSEEELQLLDLAQRKLYRDVMLENFRNVVSVGH |
| | | QSTPDGLPQLEREEKLWMMKMATQRDNSSGAKNLKEMETLQEVGLRYLP |
| 104 | ZNF175 | MSQKPQVLGPEKQDGSCEASVSFEDVTVDFSREEWQQLDPAQRCLYRDVM |
| | | LELYSHLFAVGYHIPNPEVIFRMLKEKEPRVEEAEVSHQRCQEREFGLEIPQ |
| | | KEISKKASFQ |
| 105 | ZNF595 | MELVTFRDVAIEFSPEEWKCLDPAQQNLYRDVMLENYRNLVSLGFVISNPD |
| | | LVTCLEQIKEPCNLKIHETAAKPPAICSPFSQDLSPVQGIEDSF |
| 106 | ZNF184 | MSTLLQGGHNLLSSASFQESVTFKDVIVDFTQEEWKQLDPGQRDLFRDVTL |
| | | ENYTHLVSIGLQVSKPDVISQLEQGTEPWIMEPSIPVGTCADWETRLENSVS |
| | | APEPDISEE |
| 107 | ZNF419 | MDPAQVPVAADLLTDHEEGYVTFEDVAVYFSQEEWRLLDDAQRLLYRNV |
| | | MLENFTLLASLGLASSKTHEITQLESWEEPFMPAWEVVTSAIPRGCWHGAE |
| | | AEEAPEQIASVG |
| 108 | ZFP28-1 | MKKLEAVGTGIEPKAMSQGLVTFGDVAVDFSQEEWEWLNPIQRNLYRKV |
| | | MLENYRNLASLGLCVSKPDVISSLEQGKEPWTVKRKMTRAWCPDLKAVW |
| | | KIKELPLKKDFCEG |
| 109 | ZFP28-2 | MSLLGEHWDYDALFETQPGLVTIKNLAVDFRQQLHPAQKNFCKNGIWENN |
| | | SDLGSAGHCVAKPDLVSLLEQEKEPWMVKRELTGSLFSGQRSVHETQELFP |
| | | KQDSYAE |
| 110 | ZNF18 | MLALAASQPARLEERLIRDRDLGASLLPAAPQEQWRQLDSTQKEQYWDLIL |
| | | ETYGKMVSGAGISHPKSDLTNSIEFGEELAGIYLHVNEKIPRPTCIGDRQEND |
| | | KENLNLENH |
| 111 | ZNF213 | MEGRPGETTDTCFVSGVHGPVALGDIPFYFSREEWGTLDPAQRDLFWDIKR |
| | | ENSRNTTLGFGLKGQSEKSLLQEMVPVVPGQTGSDVTVSWSPEEAEAWESE |
| | | NRPRAALGPVVGARRGRPPTRRRQFRDLA |
| 112 | ZNF394 | MVAVVRALQRALDGTSSQGMVTFEDTAVSLTWEEWERLDPARRDFCRES |
| | | AQKDSGSTVPPSLESRVENKELIPMQQILEEAEPQGQLQEAFQGKRPLFSKC |
| | | GSTHEDRVEKQSGDP |
| 113 | ZFP1 | MNKSQGSVSFTDVTVDFTQEEWEQLDPSQRILYMDVMLENYSNLLSVEVW |
| | | KADDQMERDHRNPDEQARQFLILKNQTPIEERGDLFGKALNLNTDFVSLRQ |
| | | VPYKYDLYEKTL |
| 114 | ZFP14 | MAHGSVTFRDVAIDFSQEEWEFLDPAQRDLYRDVMWENYSNFISLGPSISK |
| | | PDVITLLDEERKEPGMVVREGTRRYCPDLESRYRTNTLSPEKDIYEIYSFQW |
| | | DIMER |
| 115 | ZNF416 | MAAAVLRDSTSVPVTAEAKLMGFTQGCVTFEDVAIYFSQEEWGLLDEAQR |
| | | LLYRDVMLENFALITALVCWHGMEDEETPEQSVSVEGVPQVRTPEASPSTQ |
| | | KIQSCDMCVPFLTDILHLTDLPGQELYLTGACAVFHQDQK |
| 116 | ZNF557 | MLPPTAASQREGHTEGGELVNELLKSWLKGLVTFEDVAVEFTQEEWALLD |
| | | PAQRTLYRDVMLENCRNLASLGNQVDKPRLISQLEQEDKVMTEERGILSGT |
| | | CPDVENPFKAKGLTPKLHVFRKEQSRNMKMER |
| 117 | ZNF566 | MAQESVMFSDVSVDFSQEEWECLNDDQRDLYRDVMLENYSNLVSMGHSIS |
| | | KPNVISYLEQGKEPWLADRELTRGQWPVLESRCETKKLFLKKEIYEIESTQW |
| | | EIMEK |
| 118 | ZNF729 | MPGAPGSLEMGPLTFRDVTIEFSLEEWQCLDTVQQNLYRDVMLENYRNLV |
| | | FLGMAVFKPDLITCLKQGKEPWNMKRHEMVTKPPVMRSHFTQDLWPDQS |
| | | TKDSFQEVILRTYAR |
| 119 | ZIM2 | MAGSQFPDFKHLGTFLVFEELVTFEDVLVDFSPEELSSLSAAQRNLYREVM |
| | | LENYRNLVSLGHQFSKPDIISRLEEEESYAMETDSRHTVICQGE |
| 120 | ZNF254 | MPGPPRSLEMGLLTFRDVAIEFSLEEWQHLDIAQQNLYRNVMLENYRNLAF |
| | | LGIAVSKPDLITCLEQGKEPWNMKRHE |
| 121 | ZNF764 | MAPPLAPLPPRDPNGAGPEWREPGAVSFADVAVYFCREEWGCLRPAQRAL |
| | | YRDVMRETYGHLSALGIGGNKPALISWVEEEAELWGPAAQDPE |
| 122 | ZNF785 | MGPPLAPRPAHVPGEAGPRRTRESRPGAVSFADVAVYFSPEEWECLRPAQR |
| | | ALYRDVMRETFGHLGALGFSVPKPAFISWVEGEVEAWSPEAQDPDGESS |
| 123 | ZNF10 | MDAKSLTAWSRTLVTFKDVFVDFTREEWKLLDTAQQIVYRNVMLENYKN |
| | (KOX1) | LVSLGYQLTKPDVILRLEKGEEPWLVEREIHQETHPDSETAFEIKSSVSSRSIF |
| | | KDKQSCDIKMEGMARNDLWYLSLEEVWKCRDQLDKYQENPERHLRQVAF |
| | | TQKKVLTQERVSESGKYGGNCLLPAQLVLREYFHKRDSHTKSLKHDLVLN |
| | | GHQDSCASNSNECGQTFCQNIHLIQFARTHTGDKSYKCPDNDNSLTHGSSL |
| | | GISKGIHREKPYECKECGKFFSWRSNLTRHQLIHTGEKPYECKECGKSFSRSS |
| | | HLIGHQKTHTGEEPYECKECGKSFSWFSHLVTHQRTHTGDKLYTCNQCGKS |
| | | FVHSSRLIRHQRTHTGEKPYECPECGKSFRQSTHLILHQRTHVRVRPYECNE |
| | | CGKSYSQRSHLVVHHRIHTGLKPFECKDCGKCFSRSSHLYSHQRTHTGEKP |
| | | YECHDCGKSFSQSSALIVHQRIHTGEKPYECCQCGKAFIRKNDLIKHQRIHV |
| | | GEETYKCNQCGIIFSQNSPFIVHQIAHTGEQFLTCNQCGTALVNTSNLIGYQT |
| | | NHIRENAY |
| 124 | CBX5 | MGKKTKRTADSSSSEDEEEYVVEKVLDRRVVKGQVEYLLKWKGFSEEHNT |
| | (chromoshadow | WEPEKNLDCPELISEFMKKYKKMKEGENNKPREKSESNKRKSNFSNSADDI |
| | domain) | KSKKKREQSNDIARGFERGLEPEKIIGATDSCGDLMFLMKWKDTDEADLVL |
| | | AKEANVKCPQIVIAFYEERLTWHAYPEDAENKEKETAKS |
| 125 | RYBP | MTMGDKKSPTRPKRQAKPAADEGFWDCSVCTFRNSAEAFKCSICDVRKGT |
| | (YAF2_RYBP | STRKPRINSQLVAQQVAQQYATPPPPKKEKKEKVEKQDKEKPEKDKEISPS |
| | component | VTKKNTNKKTKPKSDILKDPPSEANSIQSANATTKTSETNHTSRPRLKNVDR |
| | of PRC1) | STAQQLAVTVGNVTVIITDFKEKTRSSSTSSSTVTSSAGSEQQNQSSSGSEST |
| | | DKGSSRSSTPKGDMSAVNDESF |
| 126 | YAF2 | MGDKKSPTRPKRQPKPSSDEGYWDCSVCTFRNSAEAFKCMMCDVRKGTST |
| | (YAF2_RYBP | RKPRPVSQLVAQQVTQQFVPPTQSKKEKKDKVEKEKSEKETTSKKNSHKK |
| | component | TRPRLKNVDRSSAQHLEVTVGDLTVIITDFKEKTKSPPASSAASADQHSQSG |
| | of PRC1) | SSSDNTERGMSRSSSPRGEASSLNGESH |
| 127 | MGA | MEEKQQIILANQDGGTVAGAAPTFFVILKQPGNGKTDQGILVTNQDACALA |
| | (component | SSVSSPVKSKGKICLPADCTVGGITVTLDNNSMWNEFYHRSTEMILTKQGR |
| | of | RMFPYCRYWITGLDSNLKYILVMDISPVDNHRYKWNGRWWEPSGKAEPH |
| | PRC1.6) | VLGRVFIHPESPSTGHYWMHQPVSFYKLKLTNNTLDQEGHIILHSMHRYLP |
| | | RLHLVPAEKAVEVIQLNGPGVHTFTFPQTEFFAVTAYQNIQITQLKIDYNPF |
| | | AKGFRDDGLNNKPQRDGKQKNSSDQEGNNISSSSGHRVRLTEGQGSEIQPG |
| | | DLDPLSRGHETSGKGLEKTSLNIKRDFLGFMDTDSALSEVPQLKQEISECLIA |
| | | SSFEDDSRVASPLDQNGSFNVVIKEEPLDDYDYELGECPEGVTVKQEETDEE |
| | | TDVYSNSDDDPILEKQLKRHNKVDNPEADHLSSKWLPSSPSGVAKAKMFK |
| | | LDTGKMPVVYLEPCAVTRSTVKISELPDNMLSTSRKDKSSMLAELEYLPTYI |
| | | ENSNETAFCLGKESENGLRKHSPDLRVVQKYPLLKEPQWKYPDISDSISTER |
| | | ILDDSKDSVGDSLSGKEDLGRKRTTMLKIATAAKVVNANQNASPNVPGKR |
| | | GRPRKLKLCKAGRPPKNTGKSLISTKNTPVSPGSTFPDVKPDLEDVDGVLFV |
| | | SFESKEALDIHAVDGTTEESSSLQASTTNDSGYRARISQLEKELIEDLKTLRH |
| | | KQVIHPGLQEVGLKLNSVDPTMSIDLKYLGVQLPLAPATSFPFWNLTGTNP |
| | | ASPDAGFPFVSRTGKTNDFTKIKGWRGKFHSASASRNEGGNSESSLKNRSA |
| | | FCSDKLDEYLENEGKLMETSMGFSSNAPTSPVVYQLPTKSTSYVRTLDSVL |
| | | KKQSTISPSTSYSLKPHSVPPVSRKAKSQNRQATFSGRTKSSYKSILPYPVSP |
| | | KQKYSHVILGDKVTKNSSGIISENQANNFVVPTLDENIFPKQISLRQAQQQQ |
| | | QQQQGSRPPGLSKSQVKLMDLEDCALWEGKPRTYITEERADVSLTTLLTAQ |
| | | ASLKTKPIHTIIRKRAPPCNNDFCRLGCVCSSLALEKRQPAHCRRPDCMFGC |
| | | TCLKRKVVLVKGGSKTKHFQRKAAHRDPVFYDTLGEEAREEEEGIREEEEQ |
| | | LKEKKKRKKLEYTICETEPEQPVRHYPLWVKVEGEVDPEPVYIPTPSVIEPM |
| | | KPLLLPQPEVLSPTVKGKLLTGIKSPRSYTPKPNPVIREEDKDPVYLYFESM |
| | | MTCARVRVYERKKEDQRQPSSSSSPSPSFQQQTSCHSSPENHNNAKEPDSEQ |
| | | QPLKQLTCDLEDDSDKLQEKSWKSSCNEGESSSTSYMHQRSPGGPTKLIEIIS |
| | | DCNWEEDRNKILSILSQHINSNMPQSLKVGSFIIELASQRKSRGEKNPPVYSS |
| | | RVKISMPSCQDQDDMAEKSGSETPDGPLSPGKMEDISPVQTDALDSVRERL |
| | | HGGKGLPFYAGLSPAGKLVAYKRKPSSSTSGLIQVASNAKVAASRKPRTLL |
| | | PSTSNSKMASSSGTATNRPGKNLKAFVPAKRPIAARPSPGGVFTQFVMSKV |
| | | GALQQKIPGVSTPQTLAGTQKFSIRPSPVMVVTPVVSSEPVQVCSPVTAAVT |
| | | TTTPQVFLENTTAVTPMTAISDVETKETTYSSGATTTGVVEVSETNTSTSVT |
| | | STQSTATVNLTKTTGITTPVASVAFPKSLVASPSTITLPVASTASTSLVVVTA |
| | | AASSSMVTTPTSSLGSVPIILSGINGSPPVSQRPENAAQIPVATPQVSPNTVKR |
| | | AGPRLLLIPVQQGSPTLRPVSNTQLQGHRMVLQPVRSPSGMNLFRHPNGQI |
| | | VQLLPLHQLRGSNTQPNLQPVMFRNPGSVMGIRLPAPSKPSETPPSSTSSSAF |
| | | SVMNPVIQAVGSSSAVNVITQAPSLLSSGASFVSQAGTLTLRISPPEPQSFAS |
| | | KTGSETKITYSSGGQPVGTASLIPLQSGSFALLQLPGQKPVPSSILQHVASLQ |
| | | MKRESQNPDQKDETNSIKREQETKKVLQSEGEAVDPEANVIKQNSGAATSE |
| | | ETLNDSLEDRGDHLDEECLPEEGCATVKPSEHSCITGSHTDQDYKDVNEEY |
| | | GARNRKSSKEKVAVLEVRTISEKASNKTVQNLSKVQHQKLGDVKVEQQKG |
| | | FDNPEENSSEFPVTFKEESKFELSGSKVMEQQSNLQPEAKEKECGDSLEKDR |
| | | ERWRKHLKGPLTRKCVGASQECKKEADEQLIKETKTCQENSDVFQQEQGIS |
| | | DLLGKSGITEDARVLKTECDSWSRISNPSAFSIVPRRAAKSSRGNGHFQGHL |
| | | LLPGEQIQPKQEKKGGRSSADFTVLDLEEDDEDDNEKTDDSIDEIVDVVSDY |
| | | QSEEVDDVEKNNCVEYIEDDEEHVDIETVEELSEEINVAHLKTTAAHTQSFK |
| | | QPSCTHISADEKAAERSRKAPPIPLKLKPDYWSDKLQKEAEAFAYYRRTHT |
| | | ANERRRRGEMRDLFEKLKITLGLLHSSKVSKSLILTRAFSEIQGLTDQADKLI |
| | | GQKNLLTRKRNILIRKVSSLSGKTEEVVLKKLEYIYAKQQALEAQKRKKKM |
| | | GSDEFDISPRISKQQEGSSASSVDLGQMFINNRRGKPLILSRKKDQATENTSP |
| | | LNTPHTSANLVMTPQGQLLTLKGPLFSGPVVAVSPDLLESDLKPQVAGSAV |
| | | ALPENDDLFMMPRIVNVTSLATEGGLVDMGGSKYPHEVPDSKPSDHLKDT |
| | | VRNEDNSLEDKGRISSRGNRDGRVTLGPTQVFLANKDSGYPQIVDVSNMQ |
| | | KAQEFLPKKISGDMRGIQYKWKESESRGERVKSKDSSFHKLKMKDLKDSSI |
| | | EMELRKVTSAIEEAALDSSELLTNMEDEDDTDETLTSLLNEIAFLNQQLNDD |
| | | SVGLAELPSSMDTEFPGDARRAFISKVPPGSRATFQVEHLGTGLKELPDVQG |
| | | ESDSISPLLLHLEDDDFSENEKQLAEPASEPDVLKIVIDSEIKDSLLSNKKAID |
| | | GGKNTSGLPAEPESVSSPPTLHMKTGLENSNSTDTLWRPMPKLAPLGLKVA |
| | | NPSSDADGQSLKVMPCLAPIAAKVGSVGHKMNLTGNDQEGRESKVMPTLA |
| | | PVVAKLGNSGASPSSAGK |
| 128 | CBX1 | MGKKQNKKKVEEVLEEEEEEYVVEKVLDRRVVKGKVEYLLKWKGFSDED |
| | (chromoshadow) | NTWEPEENLDCPDLIAEFLQSQKTAHETDKSEGGKRKADSDSEDKGEESKP |
| | | KKKKEESEKPRGFARGLEPERIIGATDSSGELMFLMKWKNSDEADLVPAKE |
| | | ANVKCPQVVISFYEERLTWHSYPSEDDDKKDDKN |
| 129 | SCMH1 | MLVCYSVLACEILWDLPCSIMGSPLGHFTWDKYLKETCSVPAPVHCFKQSY |
| | (SAM_1/SPM) | TPPSNEFKISMKLEAQDPRNTTSTCIATVVGLTGARLRLRLDGSDNKNDFW |
| | | RLVDSAEIQPIGNCEKNGGMLQPPLGFRLNASSWPMFLLKTLNGAEMAPIRI |
| | | FHKEPPSPSHNFFKMGMKLEAVDRKNPHFICPATIGEVRGSEVLVTFDGWR |
| | | GAFDYWCRFDSRDIFPVGWCSLTGDNLQPPGTKVVIPKNPYPASDVNTEKP |
| | | SIHSSTKTVLEHQPGQRGRKPGKKRGRTPKTLISHPISAPSKTAEPLKFPKKR |
| | | GPKPGSKRKPRTLLNPPPASPTTSTPEPDTSTVPQDAATIPSSAMQAPTVCIY |
| | | LNKNGSTGPHLDKKKVQQLPDHFGPARASVVLQQAVQACIDCAYHQKTVF |
| | | SFLKQGHGGEVISAVFDREQHTLNLPAVNSITYVLRFLEKLCHNLRSDNLFG |
| | | NQPFTQTHLSLTAIEYSHSHDRYLPGETFVLGNSLARSLEPHSDSMDSASNP |
| | | TNLVSTSQRHRPLLSSCGLPPSTASAVRRLCSRGVLKGSNERRDMESFWKL |
| | | NRSPGSDRYLESRDASRLSGRDPSSWTVEDVMQFVREADPQLGPHADLFRK |
| | | HEIDGKALLLLRSDMMMKYMGLKLGPALKLSYHIDRLKQGKF |
| 130 | MPP8 | MEQVAEGARVTAVPVSAADSTEELAEVEEGVGVVGEDNDAAARGAEAFG |
| | (Chromodomain) | DSEEDGEDVFEVEKILDMKTEGGKVLYKVRWKGYTSDDDTWEPEIHLEDC |
| | | KEVLLEFRKKIAENKAKAVRKDIQRLSLNNDIFEANSDSDQQSETKEDTSPK |
| | | KKKKKLRQREEKSPDDLKKKKAKAGKLKDKSKPDLESSLESLVFDLRTKK |
| | | RISEAKEELKESKKPKKDEVKETKELKKVKKGEIRDLKTKTREDPKENRKT |
| | | KKEKFVESQVESESSVLNDSPFPEDDSEGLHSDSREEKQNTKSARERAGQD |
| | | MGLEHGFEKPLDSAMSAEEDTDVRGRRKKKTPRKAEDTRENRKLENKNAF |
| | | LEKKTVPKKQRNQDRSKSAAELEKLMPVSAQTPKGRRLSGEERGLWSTDS |
| | | AEEDKETKRNESKEKYQKRHDSDKEEKGRKEPKGLKTLKEIRNAFDLFKLT |
| | | PEEKNDVSENNRKREEIPLDFKTIDDHKTKENKQSLKERRNTRDETDTWAY |
| | | IAAEGDQEVLDSVCQADENSDGRQQILSLGMDLQLEWMKLEDFQKHLDGK |
| | | DENFAATDAIPSNVLRDAVKNGDYITVKVALNSNEEYNLDQEDSSGMTLV |
| | | MLAAAGGQDDLLRLLITKGAKVNGRQKNGTTALIHAAEKNFLTTVAILLEA |
| | | GAFVNVQQSNGETALMKACKRGNSDIVRLVIECGADCNILSKHQNSALHFA |
| | | KQSNNVLVYDLLKNHLETLSRVAEETIKDYFEARLALLEPVFPIACHRLCEG |
| | | PDFSTDFNYKPPQNIPEGSGILLFIFHANFLGKEVIARLCGPCSVQAVVLNDK |
| | | FQLPVFLDSHFVYSFSPVAGPNKLFIRLTEAPSAKVKLLIGAYRVQLQ |
| 131 | SUMO3 | MSEEKPKEGVKTENDHINLKVAGQDGSVVQFKIKRHTPLSKLMKAYCERQ |
| | (Rad60- | GLSMRQIRFRFDGQPINETDTPAQLEMEDEDTIDVFQQQTGGVPESSLAGHS |
| | SLD) | F |
| 132 | HERC2 | MPSESFCLAAQARLDSKWLKTDIQLAFTRDGLCGLWNEMVKDGEIVYTGT |
| | (Cyt-b5) | ESTQNGELPPRKDDSVEPSGTKKEDLNDKEKKDEEETPAPIYRAKSILDSWV |
| | | WGKQPDVNELKECLSVLVKEQQALAVQSATTTLSALRLKQRLVILERYFIA |
| | | LNRTVFQENVKVKWKSSGISLPPVDKKSSRPAGKGVEGLARVGSRAALSFA |
| | | FAFLRRAWRSGEDADLCSELLQESLDALRALPEASLFDESTVSSVWLEVVE |
| | | RATRFLRSVVTGDVHGTPATKGPGSIPLQDQHLALAILLELAVQRGTLSQM |
| | | LSAILLLLQLWDSGAQETDNERSAQGTSAPLLPLLQRFQSIICRKDAPHSEGD |
| | | MHLLSGPLSPNESFLRYLTLPQDNELAIDLRQTAVVVMAHLDRLATPCMPP |
| | | LCSSPTSHKGSLQEVIGWGLIGWKYYANVIGPIQCEGLANLGVTQIACAEKR |
| | | FLILSRNGRVYTQAYNSDTLAPQLVQGLASRNIVKIAAHSDGHHYLALAAT |
| | | GEVYSWGCGDGGRLGHGDTVPLEEPKVISAFSGKQAGKHVVHIACGSTYS |
| | | AAITAEGELYTWGRGNYGRLGHGSSEDEAIPMLVAGLKGLKVIDVACGSG |
| | | DAQTLAVTENGQVWSWGDGDYGKLGRGGSDGCKTPKLIEKLQDLDVVKV |
| | | RCGSQFSIALTKDGQVYSWGKGDNQRLGHGTEEHVRYPKLLEGLQGKKVI |
| | | DVAAGSTHCLALTEDSEVHSWGSNDQCQHFDTLRVTKPEPAALPGLDTKHI |
| | | VGIACGPAQSFAWSSCSEWSIGLRVPFVVDICSMTFEQLDLLLRQVSEGMD |
| | | GSADWPPPQEKECVAVATLNLLRLQLHAAISHQVDPEFLGLGLGSILLNSLK |
| | | QTVVTLASSAGVLSTVQSAAQAVLQSGWSVLLPTAEERARALSALLPCAVS |
| | | GNEVNISPGRRFMIDLLVGSLMADGGLESALHAAITAEIQDIEAKKEAQKEK |
| | | EIDEQEANASTFHRSRTPLDKDLINTGICESSGKQCLPLVQLIQQLLRNIASQT |
| | | VARLKDVARRISSCLDFEQHSRERSASLDLLLRFQRLLISKLYPGESIGQTSDI |
| | | SSPELMGVGSLLKKYTALLCTHIGDILPVAASIASTSWRHFAEVAYIVEGDF |
| | | TGVLLPELVVSIVLLLSKNAGLMQEAGAVPLLGGLLEHLDRFNHLAPGKER |
| | | DDHEELAWPGIMESFFTGQNCRNNEEVTLIRKADLENHNKDGGFWTVIDG |
| | | KVYDIKDFQTQSLTGNSILAQFAGEDPVVALEAALQFEDTRESMHAFCVGQ |
| | | YLEPDQEIVTIPDLGSLSSPLIDTERNLGLLLGLHASYLAMSTPLSPVEIECAK |
| | | WLQSSIFSGGLQTSQIHYSYNEEKDEDHCSSPGGTPASKSRLCSHRRALGDH |
| | | SQAFLQAIADNNIQDHNVKDFLCQIERYCRQCHLTTPIMFPPEHPVEEVGRL |
| | | LLCCLLKHEDLGHVALSLVHAGALGIEQVKHRTLPKSVVDVCRVVYQAKC |
| | | SLIKTHQEQGRSYKEVCAPVIERLRFLFNELRPAVCNDLSIMSKFKLLSSLPR |
| | | WRRIAQKIIRERRKKRVPKKPESTDDEEKIGNEESDLEEACILPHSPINVDKR |
| | | PIAIKSPKDKWQPLLSTVTGVHKYKWLKQNVQGLYPQSPLLSTIAEFALKEE |
| | | PVDVEKMRKCLLKQLERAEVRLEGIDTILKLASKNFLLPSVQYAMFCGWQ |
| | | RLIPEGIDIGEPLTDCLKDVDLIPPFNRMLLEVTFGKLYAWAVQNIRNVLMD |
| | | ASAKFKELGIQPVPLQTITNENPSGPSLGTIPQARFLLVMLSMLTLQHGANN |
| | | LDLLLNSGMLALTQTALRLIGPSCDNVEEDMNASAQGASATVLEETRKETA |
| | | PVQLPVSGPELAAMMKIGTRVMRGVDWKWGDQDGPPPGLGRVIGELGED |
| | | GWIRVQWDTGSTNSYRMGKEGKYDLKLAELPAAAQPSAEDSDTEDDSEAE |
| | | QTERNIHPTAMMFTSTINLLQTLCLSAGVHAEIMQSEATKTLCGLLRMLVES |
| | | GTTDKTSSPNRLVYREQHRSWCTLGFVRSIALTPQVCGALSSPQWITLLMK |
| | | VVEGHAPFTATSLQRQILAVHLLQAVLPSWDKTERARDMKCLVEKLFDFL |
| | | GSLLTTCSSDVPLLRESTLRRRRVRPQASLTATHSSTLAEEVVALLRTLHSLT |
| | | QWNGLINKYINSQLRSITHSFVGRPSEGAQLEDYFPDSENPEVGGLMAVLA |
| | | VIGGIDGRLRLGGQVMHDEFGEGTVTRITPKGKITVQFSDMRTCRVCPLNQ |
| | | LKPLPAVAFNVNNLPFTEPMLSVWAQLVNLAGSKLEKHKIKKSTKQAFAG |
| | | QVDLDLLRCQQLKLYILKAGRALLSHQDKLRQILSQPAVQETGTVHTDDGA |
| | | VVSPDLGDMSPEGPQPPMILLQQLLASATQPSPVKAIFDKQELEAAALAVC |
| | | QCLAVESTHPSSPGFEDCSSSEATTPVAVQHIRPARVKRRKQSPVPALPIVVQ |
| | | LMEMGFSRRNIEFALKSLTGASGNASSLPGVEALVGWLLDHSDIQVTELSD |
| | | ADTVSDEYSDEEVVEDVDDAAYSMSTGAVVTESQTYKKRADFLSNDDYA |
| | | VYVRENIQVGMMVRCCRAYEEVCEGDVGKVIKLDRDGLHDLNVQCDWQ |
| | | QKGGTYWVRYIHVELIGYPPPSSSSHIKIGDKVRVKASVTTPKYKWGSVTH |
| | | QSVGVVKAFSANGKDIIVDFPQQSHWTGLLSEMELVPSIHPGVTCDGCQMF |
| | | PINGSRFKCRNCDDFDFCETCFKTKKHNTRHTFGRINEPGQSAVFCGRSGKQ |
| | | LKRCHSSQPGMLLDSWSRMVKSLNVSSSVNQASRLIDGSEPCWQSSGSQGK |
| | | HWIRLEIFPDVLVHRLKMIVDPADSSYMPSLVVVSGGNSLNNLIELKTININP |
| | | SDTTVPLLNDCTEYHRYIEIAIKQCRSSGIDCKIHGLILLGRIRAEEEDLAAVP |
| | | FLASDNEEEEDEKGNSGSLIRKKAAGLESAATIRTKVFVWGLNDKDQLGGL |
| | | KGSKIKVPSFSETLSALNVVQVAGGSKSLFAVTVEGKVYACGEATNGRLGL |
| | | GISSGTVPIPRQITALSSYVVKKVAVHSGGRHATALTVDGKVFSWGEGDDG |
| | | KLGHFSRMNCDKPRLIEALKTKRIRDIACGSSHSAALTSSGELYTWGLGEYG |
| | | RLGHGDNTTQLKPKMVKVLLGHRVIQVACGSRDAQTLALTDEGLVFSWG |
| | | DGDFGKLGRGGSEGCNIPQNIERLNGQGVCQIECGAQFSLALTKSGVVWT |
| | | WGKGDYFRLGHGSDVHVRKPQVVEGLRGKKIVHVAVGALHCLAVTDSGQ |
| | | VYAWGDNDHGQQGNGTTTVNRKPTLVQGLEGQKITRVACGSSHSVAWTT |
| | | VDVATPSVHEPVLFQTARDPLGASYLGVPSDADSSAASNKISGASNSKPNRP |
| | | SLAKILLSLDGNLAKQQALSHILTALQIMYARDAVVGALMPAAMIAPVECP |
| | | SFSSAAPSDASAMASPMNGEECMLAVDIEDRLSPNPWQEKREIVSSEDAVT |
| | | PSAVTPSAPSASARPFIPVTDDLGAASIIAETMTKTKEDVESQNKAAGPEPQA |
| | | LDEFTSLLIADDTRVVVDLLKLSVCSRAGDRGRDVLSAVLSGMGTAYPQV |
| | | ADMLLELCVTELEDVATDSQSGRLSSQPVVVESSHPYTDDTSTSGTVKIPGA |
| | | EGLRVEFDRQCSTERRHDPLTVMDGVNRIVSVRSGREWSDWSSELRIPGDE |
| | | LKWKFISDGSVNGWGWRFTVYPIMPAAGPKELLSDRCVLSCPSMDLVTCL |
| | | LDFRLNLASNRSIVPRLAASLAACAQLSALAASHRMWALQRLRKLLTTEFG |
| | | QSININRLLGENDGETRALSFTGSALAALVKGLPEALQRQFEYEDPIVRGGK |
| | | QLLHSPFFKVLVALACDLELDTLPCCAETHKWAWFRRYCMASRVAVALD |
| | | KRTPLPRLFLDEVAKKIRELMADSENMDVLHESHDIFKREQDEQLVQWMN |
| | | RRPDDWTLSAGGSGTIYGWGHNHRGQLGGIEGAKVKVPTPCEALATLRPV |
| | | QLIGGEQTLFAVTADGKLYATGYGAGGRLGIGGTESVSTPTLLESIQHVFIK |
| | | KVAVNSGGKHCLALSSEGEVYSWGEAEDGKLGHGNRSPCDRPRVIESLRGI |
| | | EVVDVAAGGAHSACVTAAGDLYTWGKGRYGRLGHSDSEDQLKPKLVEAL |
| | | QGHRVVDIACGSGDAQTLCLTDDDTVWSWGDGDYGKLGRGGSDGCKVP |
| | | MKIDSLTGLGVVKVECGSQFSVALTKSGAVYTWGKGDYHRLGHGSDDHV |
| | | RRPRQVQGLQGKKVIAIATGSLHCVCCTEDGEVYTWGDNDEGQLGDGTTN |
| | | AIQRPRLVAALQGKKVNRVACGSAHTLAWSTSKPASAGKLPAQVPMEYNH |
| | | LQEIPIIALRNRLLLLHHLSELFCPCIPMFDLEGSLDETGLGPSVGFDTLRGILI |
| | | SQGKEAAFRKVVQATMVRDRQHGPVVELNRIQVKRSRSKGGLAGPDGTKS |
| | | VFGQMCAKMSSFGPDSLLLPHRVWKVKFVGESVDDCGGGYSESIAEICEEL |
| | | QNGLTPLLIVTPNGRDESGANRDCYLLSPAARAPVHSSMFRFLGVLLGIAIR |
| | | TGSPLSLNLAEPVWKQLAGMSLTIADLSEVDKDFIPGLMYIRDNEATSEEFE |
| | | AMSLPFTVPSASGQDIQLSSKHTHITLDNRAEYVRLAINYRLHEFDEQVAAV |
| | | REGMARVVPVPLLSLFTGYELETMVCGSPDIPLHLLKSVATYKGIEPSASLIQ |
| | | WFWEVMESFSNTERSLFLRFVWGRTRLPRTIADFRGRDFVIQVLDKYNPPD |
| | | HFLPESYTCFFLLKLPRYSCKQVLEEKLKYAIHFCKSIDTDDYARIALTGEPA |
| | | ADDSSDDSDNEDVDSFASDSTQDYLTGH |
| 133 | BIN1 | MAEMGSKGVTAGKIASNVQKKLTRAQEKVLQKLGKADETKDEQFEQCVQ |
| | (SH3_9) | NFNKQLTEGTRLQKDLRTYLASVKAMHEASKKLNECLQEVYEPDWPGRDE |
| | | ANKIAENNDLLWMDYHQKLVDQALLTMDTYLGQFPDIKSRIAKRGRKLVD |
| | | YDSARHHYESLQTAKKKDEAKIAKPVSLLEKAAPQWCQGKLQAHLVAQT |
| | | NLLRNQAEEELIKAQKVFEEMNVDLQEELPSLWNSRVGFYVNTFQSIAGLE |
| | | ENFHKEMSKLNQNLNDVLVGLEKQHGSNTFTVKAQPSDNAPAKGNKSPSP |
| | | PDGSPAATPEIRVNHEPEPAGGATPGATLPKSPSQLRKGPPVPPPPKHTPSKE |
| | | VKQEQILSLFEDTFVPEISVTTPSQFEAPGPFSEQASLLDLDFDPLPPVTSPVK |
| | | APTPSGQSIPWDLWEPTESPAGSLPSGEPSAAEGTFAVSWPSQTAEPGPAQP |
| | | AEASEVAGGTQPAAGAQEPGETAASEAASSSLPAVVVETFPATVNGTVEGG |
| | | SGAGRLDLPPGFMFKVQAQHDYTATDTDELQLKAGDVVLVIPFQNPEEQD |
| | | EGWLMGVKESDWNQHKELEKCRGVFPENFTERVP |
| 134 | PCGF2 | MHRTTRIKITELNPHLMCALCGGYFIDATTIVECLHSFCKTCIVRYLETNKY |
| | (RING | CPMCDVQVHKTRPLLSIRSDKTLQDIVYKLVPGLFKDEMKRRRDFYAAYPL |
| | finger | TEVPNGSNEDRGEVLEQEKGALSDDEIVSLSIEFYEGARDRDEKKGPLENGD |
| | protein | GDKEKTGVRFLRCPAAMTVMHLAKFLRNKMDVPSKYKVEVLYEDEPLKE |
| | domain) | YYTLMDIAYIYPWRRNGPLPLKYRVQPACKRLTLATVPTPSEGTNTSGASE |
| | | CESVSDKAPSPATLPATSSSLPSPATPSHGSPSSHGPPATHPTSPTPPSTASGA |
| | | TTAANGGSLNCLQTPSSTSRGRKMTVNGAPVPPLT |
| 135 | TOX | MDVRFYPPPAQPAAAPDAPCLGPSPCLDPYYCNKFDGENMYMSMTEPSQD |
| | (HMG | YVPASQSYPGPSLESEDFNIPPITPPSLPDHSLVHLNEVESGYHSLCHPMNHN |
| | box) | GLLPFHPQNMDLPEITVSNMLGQDGTLLSNSISVMPDIRNPEGTQYSSHPQM |
| | | AAMRPRGQPADIRQQPGMMPHGQLTTINQSQLSAQLGLNMGGSNVPHNSP |
| | | SPPGSKSATPSPSSSVHEDEGDDTSKINGGEKRPASDMGKKPKTPKKKKKK |
| | | DPNEPQKPVSAYALFFRDTQAAIKGQNPNATFGEVSKIVASMWDGLGEEQ |
| | | KQVYKKKTEAAKKEYLKQLAAYRASLVSKSYSEPVDVKTSQPPQLINSKPS |
| | | VFHGPSQAHSALYLSSHYHQQPGMNPHLTAMHPSLPRNIAPKPNNQMPVT |
| | | VSIANMAVSPPPPLQISPPLHQHLNMQQHQPLTMQQPLGNQLPMQVQSALH |
| | | SPTMQQGFTLQPDYQTIINPTSTAAQVVTQAMEYVRSGCRNPPPQPVDWNN |
| | | DYCSSGGMQRDKALYLT |
| 136 | FOXA1 | MLGTVKMEGHETSDWNSYYADTQEAYSSVPVSNMNSGLGSMNSMNTYM |
| | (HNF3A | TMNTMTTSGNMTPASFNMSYANPGLGAGLSPGAVAGMPGGSAGAMNSM |
| | C-terminal | TAAGVTAMGTALSPSGMGAMGAQQAASMNGLGPYAAAMNPCMSPMAY |
| | domain) | APSNLGRSRAGGGGDAKTFKRSYPHAKPPYSYISLITMAIQQAPSKMLTLSE |
| | | IYQWIMDLFPYYRQNQQRWQNSIRHSLSFNDCFVKVARSPDKPGKGSYWT |
| | | LHPDSGNMFENGCYLRRQKRFKCEKQPGAGGGGGSGSGGSGAKGGPESRK |
| | | DPSGASNPSADSPLHRGVHGKTGQLEGAPAPGPAASPQTLDHSGATATGGA |
| | | SELKTPASSTAPPISSGPGALASVPASHPAHGLAPHESQLHLKGDPHYSFNHP |
| | | FSINNLMSSSEQQHKLDFKAYEQALQYSPYGSTLPASLPLGSASVTTRSPIEP |
| | | SALEPAYYQGVYSRPVLNTS |
| 137 | FOXA2 | MLGAVKMEGHEPSDWSSYYAEPEGYSSVSNMNAGLGMNGMNTYMSMSA |
| | (HNF3B | AAMGSGSGNMSAGSMNMSSYVGAGMSPSLAGMSPGAGAMAGMGGSAG |
| | C-terminal | AAGVAGMGPHLSPSLSPLGGQAAGAMGGLAPYANMNSMSPMYGQAGLSR |
| | domain) | ARDPKTYRRSYTHAKPPYSYISLITMAIQQSPNKMLTLSEIYQWIMDLFPFY |
| | | RQNQQRWQNSIRHSLSFNDCFLKVPRSPDKPGKGSFWTLHPDSGNMFENG |
| | | CYLRRQKRFKCEKQLALKEAAGAAGSGKKAAAGAQASQAQLGEAAGPAS |
| | | ETPAGTESPHSSASPCQEHKRGGLGELKGTPAAALSPPEPAPSPGQQQQAAA |
| | | HLLGPPHHPGLPPEAHLKPEHHYAFNHPFSINNLMSSEQQHHHSHHHHQPH |
| | | KMDLKAYEQVMHYPGYGSPMPGSLAMGPVTNKTGLDASPLAADTSYYQG |
| | | VYSRPIMNSS |
| 138 | IRF2BP1 | MASVQASRRQWCYLCDLPKMPWAMVWDFSEAVCRGCVNFEGADRIELLI |
| | (IRF- | DAARQLKRSHVLPEGRSPGPPALKHPATKDLAAAAAQGPQLPPPQAQPQPS |
| | 2BP1_2 N- | GTGGGVSGQDRYDRATSSGRLPLPSPALEYTLGSRLANGLGREEAVAEGAR |
| | terminal | RALLGSMPGLMPPGLLAAAVSGLGSRGLTLAPGLSPARPLFGSDFEKEKQQ |
| | domain) | RNADCLAELNEAMRGRAEEWHGRPKAVREQLLALSACAPFNVRFKKDHG |
| | | LVGRVFAFDATARPPGYEFELKLFTEYPCGSGNVYAGVLAVARQMFHDAL |
| | | REPGKALASSGFKYLEYERRHGSGEWRQLGELLTDGVRSFREPAPAEALPQ |
| | | QYPEPAPAALCGPPPRAPSRNLAPTPRRRKASPEPEGEAAGKMTTEEQQQR |
| | | HWVAPGGPYSAETPGVPSPIAALKNVAEALGHSPKDPGGGGGPVRAGGAS |
| | | PAASSTAQPPTQHRLVARNGEAEVSPTAGAEAVSGGGSGTGATPGAPLCCT |
| | | LCRERLEDTHFVQCPSVPGHKFCFPCSREFIKAQGPAGEVYCPSGDKCPLVG |
| | | SSVPWAFMQGEIATILAGDIKVKKERDP |
| 139 | IRF2BP2 | MAAAVAVAAASRRQSCYLCDLPRMPWAMIWDFTEPVCRGCVNYEGADR |
| | (IRF- | VEFVIETARQLKRAHGCFPEGRSPPGAAASAAAKPPPLSAKDILLQQQQQLG |
| | 2BP1_2 N- | HGGPEAAPRAPQALERYPLAAAAERPPRLGSDFGSSRPAASLAQPPTPQPPP |
| | terminal | VNGILVPNGFSKLEEPPELNRQSPNPRRGHAVPPTLVPLMNGSATPLPTALG |
| | domain) | LGGRAAASLAAVSGTAAASLGSAQPTDLGAHKRPASVSSSAAVEHEQREA |
| | | AAKEKQPPPPAHRGPADSLSTAAGAAELSAEGAGKSRGSGEQDWVNRPKT |
| | | VRDTLLALHQHGHSGPFESKFKKEPALTAGRLLGFEANGANGSKAVARTA |
| | | RKRKPSPEPEGEVGPPKINGEAQPWLSTSTEGLKIPMTPTSSFVSPPPPTASPH |
| | | SNRTTPPEAAQNGQSPMAALILVADNAGGSHASKDANQVHSTTRRNSNSPP |
| | | SPSSMNQRRLGPREVGGQGAGNTGGLEPVHPASLPDSSLATSAPLCCTLCH |
| | | ERLEDTHFVQCPSVPSHKFCFPCSRQSIKQQGASGEVYCPSGEKCPLVGSNV |
| | | PWAFMQGEIATILAGDVKVKKERDS |
| 140 | IRF2BPL | MSAAQVSSSRRQSCYLCDLPRMPWAMIWDFSEPVCRGCVNYEGADRIEFVI |
| | IRF- | ETARQLKRAHGCFQDGRSPGPPPPVGVKTVALSAKEAAAAAAAAAAAAA |
| | 2BP1_2 N- | AAQQQQQQQQQQQQQQQQQQQQQQQQQLNHVDGSSKPAVLAAPSGLER |
| | terminal | YGLSAAAAAAAAAAAAVEQRSRFEYPPPPVSLGSSSHTARLPNGLGGPNGF |
| | domain | PKPTPEEGPPELNRQSPNSSSAAASVASRRGTHGGLVTGLPNPGGGGGPQLT |
| | | VPPNLLPQTLLNGPASAAVLPPPPPHALGSRGPPTPAPPGAPGGPACLGGTP |
| | | GVSATSSSASSSTSSSVAEVGVGAGGKRPGSVSSTDQERELKEKQRNAEAL |
| | | AELSESLRNRAEEWASKPKMVRDTLLTLAGCTPYEVRFKKDHSLLGRVFAF |
| | | DAVSKPGMDYELKLFIEYPTGSGNVYSSASGVAKQMYQDCMKDFGRGLSS |
| | | GFKYLEYEKKHGSGDWRLLGDLLPEAVRFFKEGVPGADMLPQPYLDASCP |
| | | MLPTALVSLSRAPSAPPGTGALPPAAPSGRGAAASLRKRKASPEPPDSAEGA |
| | | LKLGEEQQRQQWMANQSEALKLTMSAGGFAAPGHAAGGPPPPPPPLGPHS |
| | | NRTTPPESAPQNGPSPMAALMSVADTLGTAHSPKDGSSVHSTTASARRNSS |
| | | SPVSPASVPGQRRLASRNGDLNLQVAPPPPSAHPGMDQVHPQNIPDSPMAN |
| | | SGPLCCTICHERLEDTHFVQCPSVPSHKFCFPCSRESIKAQGATGEVYCPSGE |
| | | KCPLVGSNVPWAFMQGEIATILAGDVKVKKERDP |
| 141 | HOXA13 | MTASVLLHPRWIEPTVMFLYDNGGGLVADELNKNMEGAAAAAAAAAAA |
| | (homeodomain) | AAAGAGGGGFPHPAAAAAGGNFSVAAAAAAAAAAAANQCRNLMAHPAP |
| | | LAPGAASAYSSAPGEAPPSAAAAAAAAAAAAAAAAAASSSGGPGPAGPAG |
| | | AEAAKQCSPCSAAAQSSSGPAALPYGYFGSGYYPCARMGPHPNAIKSCAQP |
| | | ASAAAAAAFADKYMDTAGPAAEEFSSRAKEFAFYHQGYAAGPYHHHQPM |
| | | PGYLDMPVVPGLGGPGESRHEPLGLPMESYQPWALPNGWNGQMYCPKEQ |
| | | AQPPHLWKSTLPDVVSHPSDASSYRRGRKKRVPYTKVQLKELEREYATNK |
| | | FITKDKRRRISATTNLSERQVTIWFQNRRVKEKKVINKLKTTS |
| 142 | HOXB13 | MEPGNYATLDGAKDIEGLLGAGGGRNLVAHSPLTSHPAAPTLMPAVNYAP |
| | (homeodomain) | LDLPGSAEPPKQCHPCPGVPQGTSPAPVPYGYFGGGYYSCRVSRSSLKPCA |
| | | QAATLAAYPAETPTAGEEYPSRPTEFAFYPGYPGTYQPMASYLDVSVVQTL |
| | | GAPGEPRHDSLLPVDSYQSWALAGGWNSQMCCQGEQNPPGPFWKAAFAD |
| | | SSGQHPPDACAFRRGRKKRIPYSKGQLRELEREYAANKFITKDKRRKISAAT |
| | | SLSERQITIWFQNRRVKEKKVLAKVKNSATP |
| 143 | HOXC13 | MTTSLLLHPRWPESLMYVYEDSAAESGIGGGGGGGGGGGGAGGGCSGAS |
| | (homeodomain) | PGKAPSMDGLGSSCPASHCRDLLPHPVLGRPPAPLGAPQGAVYTDIPAPEA |
| | | ARQCAPPPAPPTSSSATLGYGYPFGGSYYGCRLSHNVNLQQKPCAYHPGDK |
| | | YPEPSGALPGDDLSSRAKEFAFYPSFASSYQAMPGYLDVSVVPGISGHPEPR |
| | | HDALIPVEGYQHWALSNGWDSQVYCSKEQSQSAHLWKSPFPDVVPLQPEV |
| | | SSYRRGRKKRVPYTKVQLKELEKEYAASKFITKEKRRRISATTNLSERQVTI |
| | | WFQNRRVKEKKVVSKSKAPHLHST |
| 144 | HOXA11 | MDFDERGPCSSNMYLPSCTYYVSGPDFSSLPSFLPQTPSSRPMTYSYSSNLP |
| | (homeodomain) | QVQPVREVTFREYAIEPATKWHPRGNLAHCYSAEELVHRDCLQAPSAAGV |
| | | PGDVLAKSSANVYHHPTPAVSSNFYSTVGRNGVLPQAFDQFFETAYGTPEN |
| | | LASSDYPGDKSAEKGPPAATATSAAAAAAATGAPATSSSDSGGGGGCRET |
| | | AAAAEEKERRRRPESSSSPESSSGHTEDKAGGSSGQRTRKKRCPYTKYQIRE |
| | | LEREFFFSVYINKEKRLQLSRMLNLTDRQVKIWFQNRRMKEKKINRDRLQY |
| | | YSANPLL |
| 145 | HOXC11 | MFNSVNLGNFCSPSRKERGADFGERGSCASNLYLPSCTYYMPEFSTVSSFLP |
| | (homeodomain) | QAPSRQISYPYSAQVPPVREVSYGLEPSGKWHHRNSYSSCYAAADELMHRE |
| | | CLPPSTVTEILMKNEGSYGGHHHPSAPHATPAGFYSSVNKNSVLPQAFDRFF |
| | | DNAYCGGGDPPAEPPCSGKGEAKGEPEAPPASGLASRAEAGAEAEAEEENT |
| | | NPSSSGSAHSVAKEPAKGAAPNAPRTRKKRCPYSKFQIRELEREFFFNVYIN |
| | | KEKRLQLSRMLNLTDRQVKIWFQNRRMKEKKLSRDRLQYFSGNPLL |
| 146 | HOXC10 | MTCPRNVTPNSYAEPLAAPGGGERYSRSAGMYMQSGSDFNCGVMRGCGL |
| | (homeodomain) | APSLSKRDEGSSPSLALNTYPSYLSQLDSWGDPKAAYRLEQPVGRPLSSCSY |
| | | PPSVKEENVCCMYSAEKRAKSGPEAALYSHPLPESCLGEHEVPVPSYYRAS |
| | | PSYSALDKTPHCSGANDFEAPFEQRASLNPRAEHLESPQLGGKVSFPETPKS |
| | | DSQTPSPNEIKTEQSLAGPKGSPSESEKERAKAADSSPDTSDNEAKEEIKAEN |
| | | TTGNWLTAKSGRKKRCPYTKHQTLELEKEFLFNMYLTRERRLEISKTINLTD |
| | | RQVKIWFQNRRMKLKKMNRENRIRELTSNFNFT |
| 147 | HOXA10 | MSARKGYLLPSPNYPTTMSCSESPAANSFLVDSLISSGRGEAGGGGGGAGG |
| | (homeodomain) | GGGGGYYAHGGVYLPPAADLPYGLQSCGLFPTLGGKRNEAASPGSGGGGG |
| | | GLGPGAHGYGPSPIDLWLDAPRSCRMEPPDGPPPPPQQQPPPPPQPPQPAPQ |
| | | ATSCSFAQNIKEESSYCLYDSADKCPKVSATAAELAPFPRGPPPDGCALGTS |
| | | SGVPVPGYFRLSQAYGTAKGYGSGGGGAQQLGAGPFPAQPPGRGFDLPPA |
| | | LASGSADAARKERALDSPPPPTLACGSGGGSQGDEEAHASSSAAEELSPAPS |
| | | ESSKASPEKDSLGNSKGENAANWLTAKSGRKKRCPYTKHQTLELEKEFLFN |
| | | MYLTRERRLEISRSVHLTDRQVKIWFQNRRMKLKKMNRENRIRELTANFNF |
| | | S |
| 148 | HOXB9 | MSISGTLSSYYVDSIISHESEDAPPAKFPSGQYASSRQPGHAEHLEFPSCSFQP |
| | (homeodomain) | KAPVFGASWAPLSPHASGSLPSVYHPYIQPQGVPPAESRYLRTWLEPAPRGE |
| | | AAPGQGQAAVKAEPLLGAPGELLKQGTPEYSLETSAGREAVLSNQRPGYG |
| | | DNKICEGSEDKERPDQTNPSANWLHARSSRKKRCPYTKYQTLELEKEFLFN |
| | | MYLTRDRRHEVARLLNLSERQVKIWFQNRRMKMKKMNKEQGKE |
| 149 | HOXA9 | MATTGALGNYYVDSFLLGADAADELSVGRYAPGTLGQPPRQAATLAEHPD |
| | (homeodomain) | FSPCSFQSKATVFGASWNPVHAAGANAVPAAVYHHHHHHPYVHPQAPVA |
| | | AAAPDGRYMRSWLEPTPGALSFAGLPSSRPYGIKPEPLSARRGDCPTLDTHT |
| | | LSLTDYACGSPPVDREKQPSEGAFSENNAENESGGDKPPIDPNNPAANWLH |
| | | ARSTRKKRCPYTKHQTLELEKEFLFNMYLTRDRRYEVARLLNLTERQVKIW |
| | | FQNRRMKMKKINKDRAKDE |
| 150 | ZFP28_ | NKKLEAVGTGIEPKAMSQGLVTFGDVAVDFSQEEWEWLNPIQRNLYRKVM |
| | HUMAN | LENYRNLASLGLCVSKPDVISSLEQGKEPW |
| 151 | ZN334_ | KMKKFQIPVSFQDLTVNFTQEEWQQLDPAQRLLYRDVMLENYSNLVSVGY |
| | HUMAN | HVSKPDVIFKLEQGEEPWIVEEFSNQNYPD |
| 152 | ZN568_ | CSQESALSEEEEDTTRPLETVTFKDVAVDLTQEEWEQMKPAQRNLYRDVM |
| | HUMAN | LENYSNLVTVGCQVTKPDVIFKLEQEEEPW |
| 153 | ZN37A_ | ITSQGSVSFRDVTVGFTQEEWQHLDPAQRTLYRDVMLENYSHLVSVGYCIP |
| | UHMAN | KPEVILKLEKGEEPWILEEKFPSQSHLEL |
| 154 | ZN181_ | PQVTFNDVAIDFTHEEWGWLSSAQRDLYKDVMVQNYENLVSVAGLSVTK |
| | UHMAN | PYVITLLEDGKEPWMMEKKLSKGMIPDWESR |
| 155 | ZN510_ | PLRFSTLFQEQQKMNISQASVSFKDVTIEFTQEEWQQMAPVQKNLYRDVML |
| | HUMAN | ENYSNLVSVGYCCFKPEVIFKLEQGEEPW |
| 156 | ZN862_ | QDPSAEGLSEEVPVVFEELPVVFEDVAVYFTREEWGMLDKRQKELYRDVM |
| | HUMAN | RMNYELLASLGPAAAKPDLISKLERRAAPW |
| 157 | ZN140_ | SQGSVTFRDVAIDFSQEEWKWLQPAQRDLYRCVMLENYGHLVSLGLSISKP |
| | HUMAN | DVVSLLEQGKEPWLGKREVKRDLFSVSES |
| 158 | ZN208_ | GSLTFRDVAIEFSLEEWQCLDTAQQNLYRNVMLENYRNLVFLGIAAFKPDL |
| | HUMAN | IIFLEEGKESWNMKRHEMVEESPVICSHF |
| 159 | ZN248_ | NKSQEQVSFKDVCVDFTQEEWYLLDPAQKILYRDVILENYSNLVSVGYCIT |
| | HUMAN | KPEVIFKIEQGEEPWILEKGFPSQCHPER |
| 160 | ZN571_ | PHLLVTFRDVAIDFSQEEWECLDPAQRDLYRDVMLENYSNLISLDLESSCVT |
| | HUMAN | KKLSPEKEIYEMESLQWENMGKRINHHL |
| 161 | ZN699_ | EEERKTAELQKNRIQDSVVFEDVAVDFTQEEWALLDLAQRNLYRDVMLEN |
| | HUMAN | FQNLASLGYPLHTPHLISQWEQEEDLQTVK |
| 162 | ZN726_ | GLLTFRDVAIEFSLEEWQCLDTAQKNLYRNVMLENYRNLAFLGIAVSKPDL |
| | HUMAN | IICLEKEKEPWNMKRDEMVDEPPGICPHF |
| 163 | ZIK1_ | RAPTQVTVSPETHMDLTKGCVTFEDIAIYFSQDEWGLLDEAQRLLYLEVML |
| | MHUAN | ENFALVASLGCGHGTEDEETPSDQNVSVG |
| 164 | ZNF2_ | AAVSPTTRCQESVTFEDVAVVFTDEEWSRLVPIQRDLYKEVMLENYNSIVS |
| | MHUAN | LGLPVPQPDVIFQLKRGDKPWMVDLHGSE |
| 165 | Z705F_ | HSLEKVTFEDVAIDFTQEEWDMMDTSKRKLYRDVMLENISHLVSLGYQISK |
| | HUMAN | SYIILQLEQGKELWREGRVFLQDQNPDRE |
| 166 | ZNF14_ | DSVSFEDVAVNFTLEEWALLDSSQKKLYEDVMQETFKNLVCLGKKWEDQ |
| | HUMAN | DIEDDHRNQGKNRRCHMVERLCESRRGSKCG |
| 167 | ZN471_ | NVEVVKVMPQDLVTFKDVAIDFSQEEWQWMNPAQKRLYRSMMLENYQS |
| | HUMAN | LVSLGLCISKPYVISLLEQGREPWEMTSEMTR |
| 168 | ZN624_ | TQPDEDLHLQAEETQLVKESVTFKDVAIDFTLEEWRLMDPTQRNLHKDVM |
| | HUMAN | LENYRNLVSLGLAVSKPDMISHLENGKGPW |
| 169 | ZNF84_ | TMLQESFSFDDLSVDFTQKEWQLLDPSQKNLYKDVMLENYSSLVSLGYEV |
| | HUMAN | MKPDVIFKLEQGEEPWVGDGEIPSSDSPEV |
| 170 | ZNF7_ | EVVTFGDVAVHFSREEWQCLDPGQRALYREVMLENHSSVAGLAGFLVFKP |
| | HUMAN | ELISRLEQGEEPWVLDLQGAEGTEAPRTSK |
| 171 | ZN891_ | RNAEEERMIAVFLTTWLQEPMTFKDVAVEFTQEEWMMLDSAQRSLYRDV |
| | HUMAN | MLENYRNLTSVEYQLYRLTVISPLDQEEIRN |
| 172 | ZN337_ | GPQGARRQAFLAFGDVTVDFTQKEWRLLSPAQRALYREVTLENYSHLVSL |
| | HUMAN | GILHSKPELIRRLEQGEVPWGEERRRRPGP |
| 173 | Z705G_ | HSLKKLTFEDVAIDFTQEEWAMMDTSKRKLYRDVMLENISHLVSLGYQISK |
| | HUMAN | SYIILQLEQGKELWREGRVFLQDQNPNRE |
| 174 | ZN529_ | MPEVEFPDQFFTVLTMDHELVTLRDVVINFSQEEWEYLDSAQRNLYWDVM |
| | HUMAN | MENYSNLLSLDLESRNETKHLSVGKDIIQN |
| 175 | ZN729_ | PGAPGSLEMGPLTFRDVTIEFSLEEWQCLDTVQQNLYRDVMLENYRNLVFL |
| | HUMAN | GMAVFKPDLITCLKQGKEPWNMKRHEMVT |
| 176 | ZN419_ | RDPAQVPVAADLLTDHEEGYVTFEDVAVYFSQEEWRLLDDAQRLLYRNV |
| | HUMAN | MLENFTLLASLGLASSKTHEITQLESWEEPF |
| 177 | Z705A_ | HSLKKVTFEDVAIDFTQEEWAMMDTSKRKLYRDVMLENISHLVSLGYQISK |
| | HUMAN | SYIILQLEQGKELWREGREFLQDQNPDRE |
| 178 | ZNF45_ | TKSKEAVTFKDVAVVFSEEELQLLDLAQRKLYRDVMLENFRNVVSVGHQS |
| | HUMAN | TPDGLPQLEREEKLWMMKMATQRDNSSGAK |
| 179 | ZN302_ | SQVTFSDVAIDFSHEEWACLDSAQRDLYKDVMVQNYENLVSVGLSVTKPY |
| | HUMAN | VIMLLEDGKEPWMMEKKLSKAYPFPLSHSV |
| 180 | ZN486_ | PGPLRSLEMESLQFRDVAVEFSLEEWHCLDTAQQNLYRDVMLENYRHLVF |
| | UHMAN | LGIIVSKPDLITCLEQGIKPLTMKRHEMIA |
| 181 | ZN621_ | LQTTWPQESVTFEDVAVYFTQNQWASLDPAQRALYGEVMLENYANVASL |
| | HUMAN | VAFPFPKPALISHLERGEAPWGPDPWDTEIL |
| 182 | ZN688_ | APLLAPRPGETRPGCRKPGTVSFADVAVYFSPEEWGCLRPAQRALYRDVM |
| | HUMAN | QETYGHLGALGFPGPKPALISWMEQESEAW |
| 183 | ZN33A_ | NKVEQKSQESVSFKDVTVGFTQEEWQHLDPSQRALYRDVMLENYSNLVSV |
| | UHMAN | GYCVHKPEVIFRLQQGEEPWKQEEEFPSQS |
| 184 | ZN554_ | CFSQEERMAAGYLPRWSQELVTFEDVSMDFSQEEWELLEPAQKNLYREVM |
| | HUMAN | LENYRNVVSLEALKNQCTDVGIKEGPLSPA |
| 185 | ZN878_ | DSVAFEDVAVNFTQEEWALLDPSQKNLYREVMQETLRNLTSIGKKWNNQY |
| | HUMAN | IEDEHQNPRRNLRRLIGERLSESKESHQHG |
| 186 | ZN772_ | MGPAQVPMNSEVIVDPIQGQVNFEDVFVYFSQEEWVLLDEAQRLLYRDVM |
| | HUMAN | LENFALMASLGHTSFMSHIVASLVMGSEPW |
| 187 | ZN224_ | TTFKEAMTFKDVAVVFTEEELGLLDLAQRKLYRDVMLENFRNLLSVGHQA |
| | HUMAN | FHRDTFHFLREEKIWMMKTAIQREGNSGDK |
| 188 | ZN184_ | DSTLLQGGHNLLSSASFQEAVTFKDVIVDFTQEEWKQLDPGQRDLFRDVTL |
| | HUMAN | ENYTHLVSIGLQVSKPDVISQLEQGTEPW |
| 189 | ZN544_ | EARSMLVPPQASVCFEDVAMAFTQEEWEQLDLAQRTLYREVTLETWEHIV |
| | HUMAN | SLGLFLSKSDVISQLEQEEDLCRAEQEAPR |
| 190 | ZNF57_ | DSVVFEDVAVDFTLEEWALLDSAQRDLYRDVMLETFRNLASVDDGTQFKA |
| | HUMAN | NGSVSLQDMYGQEKSKEQTIPNFTGNNSCA |
| 191 | ZN283_ | EESHGALISSCNSRTMTDGLVTFRDVAIDFSQEEWECLDPAQRDLYVDVML |
| | HUMAN | ENYSNLVSLDLESKTYETKKIFSENDIFE |
| 192 | ZN549_ | VITPQIPMVTEEFVKPSQGHVTFEDIAVYFSQEEWGLLDEAQRCLYHDVML |
| | HUMAN | ENFSLMASVGCLHGIEAEEAPSEQTLSAQ |
| 193 | ZN211_ | VQLRPQTRMATALRDPASGSVTFEDVAVYFSWEEWDLLDEAQKHLYFDV |
| | HUMAN | MLENFALTSSLGCWCGVEHEETPSEQRISGE |
| 194 | ZN615_ | MQAQESLTLEDVAVDFTWEEWQFLSPAQKDLYRDVMLENYSNLVAVGYQ |
| | HUMAN | ASKPDALSKLERGEETCTTEDEIYSRICSEI |
| 195 | ZN253_ | GPLQFRDVAIEFSLEEWHCLDTAQRNLYRDVMLENYRNLVFLGIVVSKPDL |
| | HUMAN | VTCLEQGKKPLTMERHEMIAKPPVMSSHF |
| 196 | ZN226_ | NMFKEAVTFKDVAVAFTEEELGLLGPAQRKLYRDVMVENFRNLLSVGHPP |
| | HUMAN | FKQDVSPIERNEQLWIMTTATRRQGNLGEK |
| 197 | ZN730_ | GALTFRDVAIEFSLEEWQCLDTEQQNLYRNVMLDNYRNLVFLGIAVSKPDL |
| | HUMAN | ITCLEQEKEPWNLKTHDMVAKPPVICSHI |
| 198 | Z585A_ | SPQKSSALAPEDHGSSYEGSVSFRDVAIDFSREEWRHLDPSQRNLYRDVML |
| | HUMAN | ETYSHLLSVGYQVPEAEVVMLEQGKEPWA |
| 199 | ZN732_ | ELLTFRDVAIEFSPEEWKCLDPAQQNLYRDVMLENYRNLISLGVAISNPDLV |
| | HUMAN | IYLEQRKEPYKVKIHETVAKHPAVCSHF |
| 200 | ZN681_ | EPLKFRDVAIEFSLEEWQCLDTIQQNLYRNVMLENYRNLVFLGIVVSKPDLI |
| | HUMAN | TCLEQEKEPWTRKRHRMVAEPPVICSHF |
| 201 | ZN667_ | PSARGKSKSKAPITFGDLAIYFSQEEWEWLSPIQKDLYEDVMLENYRNLVSL |
| | HUMAN | GLSFRRPNVITLLEKGKAPWMVEPVRRR |
| 202 | ZN649_ | TKAQESLTLEDVAVDFTWEEWQFLSPAQKDLYRDVMLENYSNLVSVGYQ |
| | HUMAN | AGKPDALTKLEQGEPLWTLEDEIHSPAHPEI |
| 203 | ZN470_ | SQEEVEVAGIKLCKAMSLGSVTFTDVAIDFSQDEWEWLNLAQRSLYKKVM |
| | HUMAN | LENYRNLVSVGLCISKPDVISLLEQEKDPW |
| 204 | ZN484_ | TKSLESVSFKDVTVDFSRDEWQQLDLAQKSLYREVMLENYFNLISVGCQVP |
| | HUMAN | KPEVIFSLEQEEPCMLDGEIPSQSRPDGD |
| 205 | ZN431_ | SGCPGAERNLLVYSYFEKETLTFRDVAIEFSLEEWECLNPAQQNLYMNVML |
| | HUMAN | ENYKNLVFLGVAVSKQDPVTCLEQEKEPW |
| 206 | ZN382_ | PLQGSVSFKDVTVDFTQEEWQQLDPAQKALYRDVMLENYCHFVSVGFHM |
| | HUMAN | AKPDMIRKLEQGEELWTQRIFPSYSYLEEDG |
| 207 | ZN254_ | PGPPRSLEMGLLTFRDVAIEFSLEEWQHLDIAQQNLYRNVMLENYRNLAFL |
| | HUMAN | GIAVSKPDLITCLEQGKEPWNMKRHEMVD |
| 208 | ZN124_ | SGHPGSWEMNSVAFEDVAVNFTQEEWALLDPSQKNLYRDVMQETFRNLA |
| | HUMAN | SIGNKGEDQSIEDQYKNSSRNLRHIISHSGN |
| 209 | ZN607_ | SYGSITFGDVAIDFSHQEWEYLSLVQKTLYQEVMMENYDNLVSLAGHSVS |
| | HUMAN | KPDLITLLEQGKEPWMIVREETRGECTDLD |
| 210 | ZN317_ | DLFVCSGLEPHTPSVGSQESVTFQDVAVDFTEKEWPLLDSSQRKLYKDVML |
| | HUMAN | ENYSNLTSLGYQVGKPSLISHLEQEEEPR |
| 211 | ZN620_ | FQTAWRQEPVTFEDVAVYFTQNEWASLDSVQRALYREVMLENYANVASL |
| | HUMAN | AFPFTTPVLVSQLEQGELPWGLDPWEPMGRE |
| 212 | ZN141_ | ELLTFRDVAIEFSPEEWKCLDPDQQNLYRDVMLENYRNLVSLGVAISNPDL |
| | HUMAN | VTCLEQRKEPYNVKIHKIVARPPAMCSHF |
| 213 | ZN584_ | AGEAEAQLDPSLQGLVMFEDVTVYFSREEWGLLNVTQKGLYRDVMLENF |
| | HUMAN | ALVSSLGLAPSRSPVFTQLEDDEQSWVPSWV |
| 214 | ZN540_ | AHALVTFRDVAIDFSQKEWECLDTTQRKLYRDVMLENYNNLVSLGYSGSK |
| | HUMAN | PDVITLLEQGKEPCVVARDVTGRQCPGLLS |
| 215 | ZN75D_ | KRIKHWKMASKLILPESLSLLTFEDVAVYFSEEEWQLLNPLEKTLYNDVMQ |
| | HUMAN | DIYETVISLGLKLKNDTGNDHPISVSTSE |
| 216 | ZN555_ | DSVVFEDVAVDFTLEEWALLDSAQRDLYRDVMLETFQNLASVDDETQFKA |
| | HUMAN | SGSVSQQDIYGEKIPKESKIATFTRNVSWA |
| 217 | ZN658_ | NMSQASVSFQDVTVEFTREEWQHLGPVERTLYRDVMLENYSHLISVGYCIT |
| | HUMAN | KPKVISKLEKGEEPWSLEDEFLNQRYPGY |
| 218 | ZN684_ | ISFQESVTFQDVAVDFTAEEWQLLDCAERTLYWDVMLENYRNLISVGCPIT |
| | HUMAN | KTKVILKVEQGQEPWMVEGANPHESSPES |
| 219 | RBAK_ | NTLQGPVSFKDVAVDFTQEEWQQLDPDEKITYRDVMLENYSHLVSVGYDT |
| | HUMAN | TKPNVIIKLEQGEEPWIMGGEFPCQHSPEA |
| 220 | ZN829_ | HPEEEERMHDELLQAVSKGPVMFRDVSIDFSQEEWECLDADQMNLYKEV |
| | HUMAN | MLENFSNLVSVGLSNSKPAVISLLEQGKEPW |
| 221 | ZN582_ | SLGSELFRDVAIVFSQEEWQWLAPAQRDLYRDVMLETYSNLVSLGLAVSKP |
| | HUMAN | DVISFLEQGKEPWMVERVVSGGLCPVLES |
| 222 | ZN112_ | TKFQEMVTFKDVAVVFTEEELGLLDSVQRKLYRDVMLENFRNLLLVAHQP |
| | HUMAN | FKPDLISQLEREEKLLMVETETPRDGCSGR |
| 223 | ZN716_ | AKRPGPPGSREMGLLTFRDIAIEFSLAEWQCLDHAQQNLYRDVMLENYRNL |
| | HUMAN | VSLGIAVSKPDLITCLEQNKEPQNIKRNE |
| 224 | HKR1_ | TCMVHRQTMSCSGAGGITAFVAFRDVAVYFTQEEWRLLSPAQRTLHREVM |
| | HUMAN | LETYNHLVSLEIPSSKPKLIAQLERGEAPW |
| 225 | ZN350_ | IQAQESITLEDVAVDFTWEEWQLLGAAQKDLYRDVMLENYSNLVAVGYQ |
| | HUMAN | ASKPDALFKLEQGEQLWTIEDGIHSGACSDI |
| 226 | ZN480_ | AQKRRKRKAKESGMALPQGHLTFRDVAIEFSQAEWKCLDPAQRALYKDV |
| | HUMAN | MLENYRNLVSLGISLPDLNINSMLEQRREPW |
| 227 | ZN416_ | DSTSVPVTAEAKLMGFTQGCVTFEDVAIYFSQEEWGLLDEAQRLLYRDVM |
| | HUMAN | LENFALITALVCWHGMEDEETPEQSVSVEG |
| 228 | ZNF92_ | GPLTFRDVKIEFSLEEWQCLDTAQRNLYRDVMLENYRNLVFLGIAVSKPDLI |
| | HUMAN | TWLEQGKEPWNLKRHEMVDKTPVMCSHF |
| 229 | ZN100_ | SGCPGAERSLLVQSYFEKGPLTFRDVAIEFSLEEWQCLDSAQQGLYRKVML |
| | HUMAN | ENYRNLVFLAGIALTKPDLITCLEQGKEP |
| 230 | ZN736_ | GVLTFRDVAVEFSPEEWECLDSAQQRLYRDVMLENYGNLVSLGLAIFKPDL |
| | HUMAN | MTCLEQRKEPWKVKRQEAVAKHPAGSFHF |
| 231 | ZNF74_ | KENLEDISGWGLPEARSKESVSFKDVAVDFTQEEWGQLDSPQRALYRDVM |
| | HUMAN | LENYQNLLALGPPLHKPDVISHLERGEEPW |
| 232 | CBX1_ | EESEKPRGFARGLEPERIIGATDSSGELMFLMKWKNSDEADLVPAKEANVK |
| | HUMAN | CPQVVISFYEERLTWHSYPSEDDDKKDDK |
| 233 | ZN443_ | ASVALEDVAVNFTREEWALLGPCQKNLYKDVMQETIRNLDCVVMKWKD |
| | HUMAN | QNIEDQYRYPRKNLRCRMLERFVESKDGTQCG |
| 234 | ZN195_ | TLLTFRDVAIEFSLEEWKCLDLAQQNLYRDVMLENYRNLFSVGLTVCKPGL |
| | HUMAN | ITCLEQRKEPWNVKRQEAADGHPEMGFHH |
| 235 | ZN530_ | AAALRAPTQQVFVAFEDVAIYFSQEEWELLDEMQRLLYRDVMLENFAVM |
| | HUMAN | ASLGCWCGAVDEGTPSAESVSVEELSQGRTP |
| 236 | ZN782_ | NTFQASVSFQDVTVEFSQEEWQHMGPVERTLYRDVMLENYSHLVSVGYCF |
| | HUMAN | TKPELIFTLEQGEDPWLLEKEKGFLSRNSP |
| 237 | ZN791_ | DSVAFEDVSVSFSQEEWALLAPSQKKLYRDVMQETFKNLASIGEKWEDPN |
| | HUMAN | VEDQHKNQGRNLRSHTGERLCEGKEGSQCA |
| 238 | ZN331_ | AQGLVTFADVAIDFSQEEWACLNSAQRDLYWDVMLENYSNLVSLDLESAY |
| | HUMAN | ENKSLPTEKNIHEIRASKRNSDRRSKSLGR |
| 239 | Z354C_ | AVDLLSAQEPVTFRDVAVFFSQDEWLHLDSAQRALYREVMLENYSSLVSL |
| | HUMAN | GIPFSMPKLIHQLQQGEDPCMVEREVPSDT |
| 240 | ZN157_ | SPQRFPALIPGEPGRSFEGSVSFEDVAVDFTRQEWHRLDPAQRTMHKDVML |
| | HUMAN | ETYSNLASVGLCVAKPEMIFKLERGEELW |
| 241 | ZN727_ | RVLTFRDVAVEFSPEEWECLDSAQQRLYRDVMLENYGNLFSLGLAIFKPDL |
| | HUMAN | ITYLEQRKEPWNARRQKTVAKHPAGSLHF |
| 242 | ZN550_ | AETKDAAQMLVTFKDVAVTFTREEWRQLDLAQRTLYREVMLETCGLLVSL |
| | HUMAN | GHRVPKPELVHLLEHGQELWIVKRGLSHAT |
| 243 | ZN793_ | IEYQIPVSFKDVVVGFTQEEWHRLSPAQRALYRDVMLETYSNLVSVGYEGT |
| | HUMAN | KPDVILRLEQEEAPWIGEAACPGCHCWED |
| 244 | ZN235_ | TKFQEAVTFKDVAVAFTEEELGLLDSAQRKLYRDVMLENFRNLVSVGHQS |
| | HUMAN | FKPDMISQLEREEKLWMKELQTQRGKHSGD |
| 245 | ZNF8_ | DEGVAGVMSVGPPAARLQEPVTFRDVAVDFTQEEWGQLDPTQRILYRDVM |
| | HUMAN | LETFGHLLSIGPELPKPEVISQLEQGTELW |
| 246 | ZN724_ | GPLTFMDVAIEFSVEEWQCLDTAQQNLYRNVMLENYRNLVFLGIAVSKPD |
| | HUMAN | LITCLEQGKEPWNMERHEMVAKPPGMCCYF |
| 247 | ZN573_ | HQVGLIRSYNSKTMTCFQELVTFRDVAIDFSRQEWEYLDPNQRDLYRDVM |
| | HUMAN | LENYRNLVSLGGHSISKPVVVDLLERGKEP |
| 248 | ZN577_ | NATIVMSVRREQGSSSGEGSLSFEDVAVGFTREEWQFLDQSQKVLYKEVM |
| | HUMAN | LENYINLVSIGYRGTKPDSLFKLEQGEPPG |
| 249 | ZN789_ | FPPARGKELLSFEDVAMYFTREEWGHLNWGQKDLYRDVMLENYRNMVLL |
| | HUMAN | GFQFPKPEMICQLENWDEQWILDLPRTGNRK |
| 250 | ZN718_ | ELLTFKDVAIEFSPEEWKCLDTSQQNLYRDVMLENYRNLVSLGVSISNPDL |
| | HUMAN | VTSLEQRKEPYNLKIHETAARPPAVCSHF |
| 251 | ZN300_ | MKSQGLVSFKDVAVDFTQEEWQQLDPSQRTLYRDVMLENYSHLVSMGYP |
| | HUMAN | VSKPDVISKLEQGEEPWIIKGDISNWIYPDE |
| 252 | ZN383_ | AEGSVMFSDVSIDFSQEEWDCLDPVQRDLYRDVMLENYGNLVSMGLYTPK |
| | HUMAN | PQVISLLEQGKEPWMVGRELTRGLCSDLES |
| 253 | ZN429_ | GPLTFTDVAIEFSLEEWQCLDTAQQNLYRNVMLENYRNLVFLGIAVSKPDLI |
| | HUMAN | TCLEKEKEPCKMKRHEMVDEPPVVCSHF |
| 254 | ZN677_ | ALSQGLFTFKDVAIEFSQEEWECLDPAQRALYRDVMLENYRNLLSLDEDNI |
| | HUMAN | PPEDDISVGFTSKGLSPKENNKEELYHLV |
| 255 | ZN850_ | NMEGLVMFQDLSIDFSQEEWECLDAAQKDLYRDVMMENYSSLVSLGLSIP |
| | HUMAN | KPDVISLLEQGKEPWMVSRDVLGGWCRDSE |
| 256 | ZN454_ | AVSHLPTMVQESVTFKDVAILFTQEEWGQLSPAQRALYRDVMLENYSNLV |
| | HUMAN | SLGLLGPKPDTFSQLEKREVWMPEDTPGGF |
| 257 | ZN257_ | GPLTIRDVTVEFSLEEWHCLDTAQQNLYRDVMLENYRNLVFLGIAVSKPDL |
| | HUMAN | ITCLEQGKEPCNMKRHEMVAKPPVMCSHI |
| 258 | ZN264_ | AAAVLTDRAQVSVTFDDVAVTFTKEEWGQLDLAQRTLYQEVMLENCGLL |
| | HUMAN | VSLGCPVPKAELICHLEHGQEPWTRKEDLSQ |
| 259 | ZFP82_ | ALRSVMFSDVSIDFSPEEWEYLDLEQKDLYRDVMLENYSNLVSLGCFISKP |
| | HUMAN | DVISSLEQGKEPWKVVRKGRRQYPDLETK |
| 260 | ZFP14_ | AHGSVTFRDVAIDFSQEEWEFLDPAQRDLYRDVMWENYSNFISLGPSISKPD |
| | HUMAN | VITLLDEERKEPGMVVREGTRRYCPDLE |
| 261 | ZN485_ | APRAQIQGPLTFGDVAVAFTRIEWRHLDAAQRALYRDVMLENYGNLVSVG |
| | HUMAN | LLSSKPKLITQLEQGAEPWTEVREAPSGTH |
| 262 | ZN737_ | GPLQFRDVAIEFSLEEWHCLDTAQRNLYRNVMLENYRNLVFLGIVVSKPDL |
| | HUMAN | ITCLEQGKKPLTMKKHEMVANPSVTCSHF |
| 263 | ZNF44_ | TLPRGQPEVLEWGLPKDQDSVAFEDVAVNFTHEEWALLGPSQKNLYRDV |
| | HUMAN | MRETIRNLNCIGMKWENQNIDDQHQNLRRNP |
| 264 | ZN596_ | PSPDSMTFEDIIVDFTQEEWALLDTSQRKLFQDVMLENISHLVSIGKQLCKS |
| | HUMAN | VVLSQLEQVEKLSTQRISLLQGREVGIK |
| 265 | ZN565_ | EESREIRAGQIVLKAMAQGLVTFRDVAIEFSLEEWKCLEPAQRDLYREVTLE |
| | HUMAN | NFGHLASLGLSISKPDVVSLLEQGKEPW |
| 266 | ZN543_ | AASAQVSVTFEDVAVTFTQEEWGQLDAAQRTLYQEVMLETCGLLMSLGCP |
| | HUMAN | LFKPELIYQLDHRQELWMATKDLSQSSYPG |
| 267 | ZFP69_ | RESLEDEVTPGLPTAESQELLTFKDISIDFTQEEWGQLAPAHQNLYREVMLE |
| | HUMAN | NYSNLVSVGYQLSKPSVISQLEKGEEPW |
| 268 | SUMO1_ | EGEYIKLKVIGQDSSEIHFKVKMTTHLKKLKESYCQRQGVPMNSLRFLFEG |
| | HUMAN | QRIADNHTPKELGMEEEDVIEVYQEQTGG |
| 269 | ZNF12_ | NKSLGPVSFKDVAVDFTQEEWQQLDPEQKITYRDVMLENYSNLVSVGYHII |
| | HUMAN | KPDVISKLEQGEEPWIVEGEFLLQSYPDE |
| 270 | ZN169_ | SPGLLTTRKEALMAFRDVAVAFTQKEWKLLSSAQRTLYREVMLENYSHLV |
| | HUMAN | SLGIAFSKPKLIEQLEQGDEPWREENEHLL |
| 271 | ZN433_ | MFQDSVAFEDVAVTFTQEEWALLDPSQKNLCRDVMQETFRNLASIGKKWK |
| | HUMAN | PQNIYVEYENLRRNLRIVGERLFESKEGHQ |
| 272 | SUMO3_ | ENDHINLKVAGQDGSVVQFKIKRHTPLSKLMKAYCERQGLSMRQIRFRFDG |
| | HUMAN | QPINETDTPAQLEMEDEDTIDVFQQQTGG |
| 273 | ZNF98_ | PGPLGSLEMGVLTFRDVALEFSLEEWQCLDTAQQNLYRNVMLENYRNLVF |
| | HUMAN | VGIAASKPDLITCLEQGKEPWNVKRHEMVT |
| 274 | ZN175_ | LSQKPQVLGPEKQDGSCEASVSFEDVTVDFSREEWQQLDPAQRCLYRDVM |
| | HUMAN | LELYSHLFAVGYHIPNPEVIFRMLKEKEPR |
| 275 | ZN347_ | ALTQGQVTFRDVAIEFSQEEWTCLDPAQRTLYRDVMLENYRNLASLGISCF |
| | HUMAN | DLSIISMLEQGKEPFTLESQVQIAGNPDG |
| 276 | ZNF25_ | NKFQGPVTLKDVIVEFTKEEWKLLTPAQRTLYKDVMLENYSHLVSVGYHV |
| | HUMAN | NKPNAVFKLKQGKEPWILEVEFPHRGFPED |
| 277 | ZN519_ | ELLTFRDVAIEFSPEEWKCLDPAQQNLYRDVMLENYRNLVSLAVYSYYNQ |
| | HUMAN | GILPEQGIQDSFKKATLGRYGSCGLENICL |
| 278 | Z585B_ | SPQKSSALAPEDHGSSYEGSVSFRDVAIDFSREEWRHLDLSQRNLYRDVML |
| | HUMAN | ETYSHLLSVGYQVPKPEVVMLEQGKEPWA |
| 279 | ZIM3_ | NNSQGRVTFEDVTVNFTQGEWQRLNPEQRNLYRDVMLENYSNLVSVGQG |
| | HUMAN | ETTKPDVILRLEQGKEPWLEEEEVLGSGRAE |
| 280 | ZN517_ | AMALPMPGPQEAVVFEDVAVYFTRIEWSCLAPDQQALYRDVMLENYGNL |
| | HUMAN | ASLGFLVAKPALISLLEQGEEPGALILQVAE |
| 281 | ZN846_ | DSSQHLVTFEDVAVDFTQEEWTLLDQAQRDLYRDVMLENYKNLIILAGSEL |
| | HUMAN | FKRSLMSGLEQMEELRTGVTGVLQELDLQ |
| 282 | ZN230_ | TTFKEAVTFKDVAVFFTEEELGLLDPAQRKLYQDVMLENFTNLLSVGHQPF |
| | HUMAN | HPFHFLREEKFWMMETATQREGNSGGKTI |
| 283 | ZNF66_ | GPLQFRDVAIEFSLEEWHCLDMAQRNLYRDVMLENYRNLVFLGIVVSKPD |
| | HUMAN | LITHLEQGKKPSTMQRHEMVANPSVLCSHF |
| 284 | ZFP1_ | NKSQGSVSFTDVTVDFTQEEWEQLDPSQRILYMDVMLENYSNLLSVEVWK |
| | HUMAN | ADDQMERDHRNPDEQARQFLILKNQTPIEE |
| 285 | ZN713_ | EEEEMNDGSQMVRSQESLTFQDVAVDFTREEWDQLYPAQKNLYRDVMLE |
| | HUMAN | NYRNLVALGYQLCKPEVIAQLELEEEWVIER |
| 286 | ZN816_ | EEATKKSKEKEPGMALPQGRLTFRDVAIEFSLEEWKCLNPAQRALYRAVM |
| | HUMAN | LENYRNLEFVDSSLKSMMEFSSTRHSITGE |
| 287 | ZN426_ | EKTPAGRIVADCLTDCYQDSVTFDDVAVDFTQEEWTLLDSTQRSLYSDVM |
| | HUMAN | LENYKNLATVGGQIIKPSLISWLEQEESRT |
| 288 | ZN674_ | AMSQESLTFKDVFVDFTLEEWQQLDSAQKNLYRDVMLENYSHLVSVGHL |
| | HUMAN | VGKPDVIFRLGPGDESWMADGGTPVRTCAGE |
| 289 | ZN627_ | DSVAFEDVAVNFTLEEWALLDPSQKNLYRDVMRETFRNLASVGKQWEDQ |
| | HUMAN | NIEDPFKIPRRNISHIPERLCESKEGGQGEE |
| 290 | ZNF20_ | MFQDSVAFEDVAVSFTQEEWALLDPSQKNLYRDVMQETFKNLTSVGKTW |
| | HUMAN | KVQNIEDEYKNPRRNLSLMREKLCESKESHH |
| 291 | Z587B_ | AVVATLRLSAQGTVTFEDVAVKFTQEEWNLLSEAQRCLYRDVTLENLALM |
| | HUMAN | SSLGCWCGVEDEAAPSKQSIYIQRETQVRT |
| 292 | ZN316_ | EEEEEDEDEDDLLTAGCQELVTFEDVAVYFSLEEWERLEADQRGLYQEVM |
| | HUMAN | QENYGILVSLGYPIPKPDLIFRLEQGEEPW |
| 293 | ZN233_ | TKFQEMVTFKDVAVVFTREELGLLDLAQRKLYQDVMLENFRNLLSVGYQP |
| | HUMAN | FKLDVILQLGKEDKLRMMETEIQGDGCSGH |
| 294 | ZN611_ | EEAAQKRKGKEPGMALPQGRLTFRDVAIEFSLAEWKCLNPSQRALYREVM |
| | HUMAN | LENYRNLEAVDISSKCMMKEVLSTGQGNTE |
| 295 | ZN556_ | DTVVFEDVVVDFTLEEWALLNPAQRKLYRDVMLETFKHLASVDNEAQLK |
| | HUMAN | ASGSISQQDTSGEKLSLKQKIEKFTRKNIWA |
| 296 | ZN234_ | TTFKEGLTFKDVAVVFTEEELGLLDPVQRNLYQDVMLENFRNLLSVGHHPF |
| | HUMAN | KHDVFLLEKEKKLDIMKTATQRKGKSADK |
| 297 | ZN560_ | SALQQEFWKIQTSNGIQMDLVTFDSVAVEFTQEEWTLLDPAQRNLYSDVM |
| | HUMAN | LENYKNLSSVGYQLFKPSLISWLEEEEELS |
| 298 | ZNF77_ | DCVIFEEVAVNFTPEEWALLDHAQRSLYRDVMLETCRNLASLDCYIYVRTS |
| | HUMAN | GSSSQRDVFGNGISNDEEIVKFTGSDSWS |
| 299 | ZN682_ | ELLTFRDVTIEFSLEEWEFLNPAQQSLYRKVMLENYRNLVSLGLTVSKPELI |
| | HUMAN | SRLEQRQEPWNVKRHETIAKPPAMSSHY |
| 300 | ZN614_ | IKTQESLTLEDVAVEFSWEEWQLLDTAQKNLYRDVMVENYNHLVSLGYQT |
| | HUMAN | SKPDVLSKLAHGQEPWTTDAKIQNKNCPGI |
| 301 | ZN785_ | PAHVPGEAGPRRTRESRPGAVSFADVAVYFSPEEWECLRPAQRALYRDVM |
| | HUMAN | RETFGHLGALGFSVPKPAFISWVEGEVEAW |
| 302 | ZN445_ | GCPGDQVTPTRSLTAQLQETMTFKDVEVTFSQDEWGWLDSAQRNLYRDV |
| | HUMAN | MLENYRNMASLVGPFTKPALISWLEAREPWG |
| 303 | ZFP30_ | ARDLVMFRDVAVDFSQEEWECLNSYQRNLYRDVILENYSNLVSLAGCSISK |
| | HUMAN | PDVITLLEQGKEPWMVVRDEKRRWTLDLE |
| 304 | ZN225_ | TTLKEAVTFKDVAVVFTEEELRLLDLAQRKLYREVMLENFRNLLSVGHQSL |
| | HUMAN | HRDTFHFLKEEKFWMMETATQREGNLGGK |
| 305 | ZN551_ | SPPSPRSSMAAVALRDSAQGMTFEDVAIYFSQEEWELLDESQRFLYCDVML |
| | HUMAN | ENFAHVTSLGYCHGMENEAIASEQSVSIQ |
| 306 | ZN610_ | DEEAQKRKAKESGMALPQGRLTFMDVAIEFSQEEWKSLDPGQRALYRDV |
| | HUMAN | MLENYRNLVFLGICLPDLSIISMLKQRREPL |
| 307 | ZN528_ | ALTQGPLKFMDVAIEFSQEEWKCLDPAQRTLYRDVMLENYRNLVSLGICLP |
| | HUMAN | DLSVTSMLEQKRDPWTLQSEEKIANDPDG |
| 308 | ZN284_ | TMFKEAVTFKDVAVVFTEEELGLLDVSQRKLYRDVMLENFRNLLSVGHQL |
| | HUMAN | SHRDTFHFQREEKFWIMETATQREGNSGGK |
| 309 | ZN418_ | QGTVAFEDVAVNFSQEEWSLLSEVQRCLYHDVMLENWVLISSLGCWCGSE |
| | HUMAN | DEEAPSKKSISIQRVSQVSTPGAGVSPKKA |
| 310 | MPP8_ | AEAFGDSEEDGEDVFEVEKILDMKTEGGKVLYKVRWKGYTSDDDTWEPEI |
| | HUMAN | HLEDCKEVLLEFRKKIAENKAKAVRKDIQR |
| 311 | ZN490_ | VLQMQNSEHHGQSIKTQTDSISLEDVAVNFTLEEWALLDPGQRNIYRDVMR |
| | HUMAN | ATFKNLACIGEKWKDQDIEDEHKNQGRNL |
| 312 | ZN805_ | AMALTDPAQVSVTFDDVAVTFTQEEWGQLDLAQRTLYQEVMLENCGLLV |
| | HUMAN | SLGCPVPRPELIYHLEHGQEPWTRKEDLSQG |
| 313 | Z780B_ | VHGSVTFRDVAIDFSQEEWECLQPDQRTLYRDVMLENYSHLISLGSSISKPD |
| | HUMAN | VITLLEQEKEPWIVVSKETSRWYPDLES |
| 314 | ZN763_ | DPVACEDVAVNFTQEEWALLDISQRKLYREVMLETFRNLTSIGKKWKDQNI |
| | HUMAN | EYEYQNPRRNFRSLIEGNVNEIKEDSHCG |
| 315 | ZN285_ | IKFQERVTFKDVAVVFTKEELALLDKAQINLYQDVMLENFRNLMLVRDGIK |
| | HUMAN | NNILNLQAKGLSYLSQEVLHCWQIWKQRI |
| 316 | ZNF85_ | GPLTFRDVAIEFSLKEWQCLDTAQRNLYRNVMLENYRNLVFLGITVSKPDLI |
| | HUMAN | TCLEQGKEAWSMKRHEIMVAKPTVMCSH |
| 317 | ZN223_ | TMSKEAVTFKDVAVVFTEEELGLLDLAQRKLYRDVMLENFRNLLSVGHQP |
| | HUMAN | FHRDTFHFLREEKFWMMDIATQREGNSGGK |
| 318 | ZNF90_ | GPLEFRDVAIEFSLEEWHCLDTAQQNLYRDVMLENYRHLVFLGIVVTKPDL |
| | HUMAN | ITCLEQGKKPFTVKRHEMIAKSPVMCFHF |
| 319 | ZN557_ | GHTEGGELVNELLKSWLKGLVTFEDVAVEFTQEEWALLDPAQRTLYRDV |
| | HUMAN | MLENCRNLASLGNQVDKPRLISQLEQEDKVM |
| 320 | ZN425_ | AEPASVTVTFDDVALYFSEQEWEILEKWQKQMYKQEMKTNYETLDSLGY |
| | HUMAN | AFSKPDLITWMEQGRMLLISEQGCLDKTRRT |
| 321 | ZN229_ | HSQASAISQDREEKIMSQEPLSFKDVAVVFTEEELELLDSTQRQLYQDVMQ |
| | HUMAN | ENFRNLLSVGERNPLGDKNGKDTEYIQDE |
| 322 | ZN606_ | GSLEEGRRATGLPAAQVQEPVTFKDVAVDFTQEEWGQLDLVQRTLYRDV |
| | HUMAN | MLETYGHLLSVGNQIAKPEVISLLEQGEEPW |
| 323 | ZN155_ | TTFKEAVTFKDVAVVFTEEELGLLDPAQRKLYRDVMLENFRNLLSVGHQPF |
| | HUMAN | HQDTCHFLREEKFWMMGTATQREGNSGGK |
| 324 | ZN222_ | AKLYEAVTFKDVAVIFTEEELGLLDPAQRKLYRDVMLENFRNLLSVGGKIQ |
| | HUMAN | TEMETVPEAGTHEEFSCKQIWEQIASDLT |
| 325 | ZN442_ | RSDLFLPDSQTNEERKQYDSVAFEDVAVNFTQEEWALLGPSQKSLYRDVM |
| | HUMAN | WETIRNLDCIGMKWEDTNIEDQHRNPRRSL |
| 326 | ZNF91_ | PGTPGSLEMGLLTFRDVAIEFSPEEWQCLDTAQQNLYRNVMLENYRNLAFL |
| | HUMAN | GIALSKPDLITYLEQGKEPWNMKQHEMVD |
| 327 | ZN135_ | TPGVRVSTDPEQVTFEDVVVGFSQEEWGQLKPAQRTLYRDVMLDTFRLLV |
| | HUMAN | SVGHWLPKPNVISLLEQEAELWAVESRLPQ |
| 328 | ZN778_ | EQTQAAGMVAGWLINCYQDAVTFDDVAVDFTQEEWTLLDPSQRDLYRDV |
| | HUMAN | MLENYENLASVEWRLKTKGPALRQDRSWFRA |
| 329 | RYBP_ | PSEANSIQSANATTKTSETNHTSRPRLKNVDRSTAQQLAVTVGNVTVIITDF |
| | HUMAN | KEKTRSSSTSSSTVTSSAGSEQQNQSSS |
| 330 | ZN534_ | ALTQGQLSFSDVAIEFSQEEWKCLDPGQKALYRDVMLENYRNLVSLGEDN |
| | HUMAN | VRPEACICSGICLPDLSVTSMLEQKRDPWT |
| 331 | ZN586_ | AAAAALRAPAQSSVTFEDVAVNFSLEEWSLLNEAQRCLYRDVMLETLTLIS |
| | HUMAN | SLGCWHGGEDEAAPSKQSTCIHIYKDQGG |
| 332 | ZN567_ | AQGSVSFNDVTVDFTQEEWQHLDHAQKTLYMDVMLENYCHLISVGCHMT |
| | HUMAN | KPDVILKLERGEEPWTSFAGHTCLEENWKAE |
| 333 | ZN440_ | DPVAFKDVAVNFTQEEWALLDISQRKLYREVMLETFRNLTSLGKRWKDQN |
| | HUMAN | IEYEHQNPRRNFRSLIEEKVNEIKDDSHCG |
| 334 | ZN583_ | SKDLVTFGDVAVNFSQEEWEWLNPAQRNLYRKVMLENYRSLVSLGVSVS |
| | HUMAN | KPDVISLLEQGKEPWMVKKEGTRGPCPDWEY |
| 335 | ZN441_ | DSVAFEDVAINFTCEEWALLGPSQKSLYRDVMQETIRNLDCIGMIWQNHDI |
| | HUMAN | EEDQYKDLRRNLRCHMVERACEIKDNSQC |
| 336 | ZNF43_ | GPLTFMDVAIEFCLEEWQCLDIAQQNLYRNVMLENYRNLVFLGIAVSKPDL |
| | HUMAN | ITCLEQEKEPWEPMRRHEMVAKPPVMCSH |
| 337 | CBX5_ | QSNDIARGFERGLEPEKIIGATDSCGDLMFLMKWKDTDEADLVLAKEANV |
| | HUMAN | KCPQIVIAFYEERLTWHAYPEDAENKEKET |
| 338 | ZN589_ | ALPAKDSAWPWEEKPRYLGPVTFEDVAVLFTEAEWKRLSLEQRNLYKEVM |
| | HUMAN | LENLRNLVSLAESKPEVHTCPSCPLAFGSQ |
| 339 | ZNF10_ | DAKSLTAWSRTLVTFKDVFVDFTREEWKLLDTAQQIVYRNVMLENYKNLV |
| | HUMAN | SLGYQLTKPDVILRLEKGEEPWLVEREIHQ |
| 340 | ZN563_ | DAVAFEDVAVNFTQEEWALLGPSQKNLYRYVMQETIRNLDCIRMIWEEQN |
| | HUMAN | TEDQYKNPRRNLRCHMVERFSESKDSSQCG |
| 341 | ZN561_ | EKTKVERMVEDYLASGYQDSVTFDDVAVDFTPEEWALLDTTEKYLYRDV |
| | HUMAN | MLENYMNLASVEWEIQPRTKRSSLQQGFLKN |
| 342 | ZN136_ | DSVAFEDVDVNFTQEEWALLDPSQKNLYRDVMWETMRNLASIGKKWKDQ |
| | HUMAN | NIKDHYKHRGRNLRSHMLERLYQTKDGSQRG |
| 343 | ZN630_ | IESQEPVTFEDVAVDFTQEEWQQLNPAQKTLHRDVMLETYNHLVSVGCSGI |
| | HUMAN | KPDVIFKLEHGKDPWIIESELSRWIYPDR |
| 344 | ZN527_ | AVGLCKAMSQGLVTFRDVALDFSQEEWEWLKPSQKDLYRDVMLENYRNL |
| | HUMAN | VWLGLSISKPNMISLLEQGKEPWMVERKMSQ |
| 345 | ZN333_ | DKVEEEAMAPGLPTACSQEPVTFADVAVVFTPEEWVFLDSTQRSLYRDVM |
| | HUMAN | LENYRNLASVADQLCKPNALSYLEERGEQW |
| 346 | Z324B_ | TFEDVAVYFSQEEWGLLDTAQRALYRHVMLENFTLVTSLGLSTSRPRVVIQ |
| | HUMAN | LERGEEPWVPSGKDMTLARNTYGRLNSGS |
| 347 | ZN786_ | AEPPRLPLTFEDVAIYFSEQEWQDLEAWQKELYKHVMRSNYETLVSLDDG |
| | HUMAN | LPKPELISWIEHGGEPFRKWRESQKSGNII |
| 348 | ZN709_ | DSVVFEDVAVNFTQEEWALLGPSQKKLYRDVMQETFVNLASIGENWEEKN |
| | HUMAN | IEDHKNQGRKLRSHMVERLCERKEGSQFGE |
| 349 | ZN792_ | AAAALRDPAQGCVTFEDVTIYFSQEEWVLLDEAQRLLYCDVMLENFALIAS |
| | HUMAN | LGLISFRSHIVSQLEMGKEPWVPDSVDMT |
| 350 | ZN599_ | AAPALALVSFEDVVVTFTGEEWGHLDLAQRTLYQEVMLETCRLLVSLGHP |
| | HUMAN | VPKPELIYLLEHGQELWTVKRGLSQSTCAG |
| 351 | ZN613_ | IKSQESLTLEDVAVEFTWEEWQLLGPAQKDLYRDVMLENYSNLVSVGYQA |
| | HUMAN | SKPDALFKLEQGEPWTVENEIHSQICPEIK |
| 352 | ZF69B_ | GESLESRVTLGSLTAESQELLTFKDVSVDFTQEEWGQLAPAHRNLYREVML |
| | HUMAN | ENYGNLVSVGCQLSKPGVISQLEKGEEPW |
| 353 | ZN799_ | ASVALEDVAVNFTREEWALLGPCQKNLYKDVMQETIRNLDCVGMKWKD |
| | HUMAN | QNIEDQYRYPRKNLRCRMLERFVESKDGTQCG |
| 354 | ZN569_ | TESQGTVTFKDVAIDFTQEEWKRLDPAQRKLYRNVMLENYNNLITVGYPFT |
| | HUMAN | KPDVIFKLEQEEEPWVMEEEVLRRHWQGE |
| 355 | ZN564_ | DSVASEDVAVNFTLEEWALLDPSQKKLYRDVMRETFRNLACVGKKWEDQ |
| | HUMAN | SIEDWYKNQGRILRNHMEEGLSESKEYDQCG |
| 356 | ZN546_ | EETQGELTSSCGSKTMANVSLAFRDVSIDLSQEEWECLDAVQRDLYKDVM |
| | HUMAN | LENYSNLVALGYTIPKPDVITLLEQEKEPW |
| 357 | ZFP92_ | AAILLTTRPKVPVSFEDVSVYFTKTEWKLLDLRQKVLYKRVMLENYSHLVS |
| | HUMAN | LGFSFSKPHLISQLERGEGPWVADIPRTW |
| 358 | YAF2_ | KDKVEKEKSEKETTSKKNSHKKTRPRLKNVDRSSAQHLEVTVGDLTVIITD |
| | HUMAN | FKEKTKSPPASSAASADQHSQSGSSSDNT |
| 359 | ZN723_ | GPLTFTDVAIKFSLEEWQFLDTAQQNLYRDVMLENYRNLVFLGVGVSKPD |
| | HUMAN | LITCLEQGKEPWNMKRHKMVAKPPVVCSHF |
| 360 | ZNF34_ | RKPNPQAMAALFLSAPPQAEVTFEDVAVYLSREEWGRLGPAQRGLYRDVM |
| | HUMAN | LETYGNLVSLGVGPAGPKPGVISQLERGDE |
| 361 | ZN439_ | LSLSPILLYTCEMFQDPVAFKDVAVNFTQEEWALLDISQKNLYREVMLETF |
| | HUMAN | WNLTSIGKKWKDQNIEYEYQNPRRNFRSV |
| 362 | ZFP57_ | AAGEPRSLLFFQKPVTFEDVAVNFTQEEWDCLDASQRVLYQDVMSETFKN |
| | HUMAN | LTSVARIFLHKPELITKLEQEEEQWRETRV |
| 363 | ZNF19_ | AAMPLKAQYQEMVTFEDVAVHFTKTEWTGLSPAQRALYRSVMLENFGNL |
| | HUMAN | TALGYPVPKPALISLLERGDMAWGLEAQDDP |
| 364 | ZN404_ | ARVPLTFSDVAIDFSQEEWEYLNSDQRDLYRDVMLENYTNLVSLDFNFTTE |
| | HUMAN | SNKLSSEKRNYEVNAYHQETWKRNKTFNL |
| 365 | ZN274_ | ASRLPTAWSCEPVTFEDVTLGFTPEEWGLLDLKQKSLYREVMLENYRNLVS |
| | HUMAN | VEHQLSKPDVVSQLEEAEDFWPVERGIPQ |
| 366 | CBX3_ | SKKKRDAADKPRGFARGLDPERIIGATDSSGELMFLMKWKDSDEADLVLA |
| | HUMAN | KEANMKCPQIVIAFYEERLTWHSCPEDEAQ |
| 367 | ZNF30_ | AHKYVGLQYHGSVTFEDVAIAFSQQEWESLDSSQRGLYRDVMLENYRNLV |
| | HUMAN | SMGHSRSKPHVIALLEQWKEPEVTVRKDGR |
| 368 | ZN250_ | AAARLLPVPAGPQPLSFQAKLTFEDVAVLLSQDEWDRLCPAQRGLYRNVM |
| | HUMAN | METYGNVVSLGLPGSKPDIISQLERGEDPW |
| 369 | ZN570_ | AVGLLKAMYQELVTFRDVAVDFSQEEWDCLDSSQRHLYSNVMLENYRILV |
| | HUMAN | SLGLCFSKPSVILLLEQGKAPWMVKRELTK |
| 370 | ZN675_ | GLLTFRDVAIEFSLEEWQCLDTAQRNLYKNVILENYRNLVFLGIAVSKQDLI |
| | HUMAN | TCLEQEKEPLTVKRHEMVNEPPVMCSHF |
| 371 | ZN695_ | GLLAFRDVALEFSPEEWECLDPAQRSLYRDVMLENYRNLISLGEDSFNMQF |
| | HUMAN | LFHSLAMSKPELIICLEARKEPWNVNTEK |
| 372 | ZN548_ | NLTEGRVVFEDVAIYFSQEEWGHLDEAQRLLYRDVMLENLALLSSLGSWH |
| | HUMAN | GAEDEEAPSQQGFSVGVSEVTASKPCLSSQ |
| 373 | ZN132_ | GPAQHTSWPCGSAVPTLKSMVTFEDVAVYFSQEEWELLDAAQRHLYHSV |
| | HUMAN | MLENLELVTSLGSWHGVEGEGAHPKQNVSVE |
| 374 | ZN738_ | SGYPGAERNLLEYSYFEKGPLTFRDVVIEFSQEEWQCLDTAQQDLYRKVML |
| | HUMAN | ENFRNLVFLGIDVSKPDLITCLEQGKDPW |
| 375 | ZN420_ | ARKLVMFRDVAIDFSQEEWECLDSAQRDLYRDVMLENYSNLVSLDLPSRC |
| | HUMAN | ASKDLSPEKNTYETELSQWEMSDRLENCDL |
| 376 | ZN626_ | GPLQFRDVAIEFSLEEWHCLDTAQRNLYRNVMLENYSNLVFLGITVSKPDLI |
| | HUMAN | TCLEQGRKPLTMKRNEMIAKPSVMCSHF |
| 377 | ZN559_ | VAGWLTNYSQDSVTFEDVAVDFTQEEWTLLDQTQRNLYRDVMLENYKNL |
| | HUMAN | VAVDWESHINTKWSAPQQNFLQGKTSSVVEM |
| 378 | ZN460_ | AAAWMAPAQESVTFEDVAVTFTQEEWGQLDVTQRALYVEVMLETCGLLV |
| | HUMAN | ALGDSTKPETVEPIPSHLALPEEVSLQEQLA |
| 379 | ZN268_ | VLEWLFISQEQPKITKSWGPLSFMDVFVDFTWEEWQLLDPAQKCLYRSVM |
| | HUMAN | LENYSNLVSLGYQHTKPDIIFKLEQGEELC |
| 380 | ZN304_ | AAAVLMDRVQSCVTFEDVFVYFSREEWELLEEAQRFLYRDVMLENFALVA |
| | HUMAN | TLGFWCEAEHEAPSEQSVSVEGVSQVRTAE |
| 381 | ZIM2_ | AGSQFPDFKHLGTFLVFEELVTFEDVLVDFSPEELSSLSAAQRNLYREVMLE |
| | HUMAN | NYRNLVSLGHQFSKPDIISRLEEEESYA |
| 382 | ZN605_ | IQSQISFEDVAVDFTLEEWQLLNPTQKNLYRDVMLENYSNLVFLEVWLDNP |
| | HUMAN | KMWLRDNQDNLKSMERGHKYDVFGKIFNS |
| 383 | ZN844_ | DLVAFEDVAVNFTQEEWSLLDPSQKNLYREVMQETLRNLASIGEKWKDQN |
| | HUMAN | IEDQYKNPRNNLRSLLGERVDENTEENHCG |
| 384 | SUMO5_ | KDEDIKLRVIGQDSSEIHFKVKMTTPLKKLKKSYCQRQGVPVNSLRFLFEGQ |
| | HUMAN | RIADNHTPEELGMEEEDVIEVYQEQIGG |
| 385 | ZN101_ | DSVAFEDVAVNFTQEEWALLSPSQKNLYRDVTLETFRNLASVGIQWKDQDI |
| | HUMAN | ENLYQNLGIKLRSLVERLCGRKEGNEHRE |
| 386 | ZN783_ | RNFWILRLPPGSKGEAPKVPVTFDDVAVYFSELEWGKLEDWQKELYKHVM |
| | HUMAN | RGNYETLVSLDYAISKPDILTRIERGEEPC |
| 387 | ZN417_ | AAAAPRRPTQQGTVTFEDVAVNFSQEEWCLLSEAQRCLYRDVMLENLALIS |
| | HUMAN | SLGCWCGSKDEEAPCKQRISVQRESQSRT |
| 388 | ZN182_ | SGEDSGSFYSWQKAKREQGLVTFEDVAVDFTQEEWQYLNPPQRTLYRDV |
| | HUMAN | MLETYSNLVFVGQQVTKPNLILKLEVEECPA |
| 389 | ZN823_ | DSVAFEDVAVNFTQEEWALLGPSQKSLYRNVMQETIRNLDCIEMKWEDQN |
| | HUMAN | IGDQCQNAKRNLRSHTCEIKDDSQCGETFG |
| 390 | ZN177_ | AAGWLTTWSQNSVTFQEVAVDFSQEEWALLDPAQKNLYKDVMLENFRNL |
| | HUMAN | ASVGYQLCRHSLISKVDQEQLKTDERGILQG |
| 391 | ZN197_ | ENPRNQLMALMLLTAQPQELVMFEEVSVCFTSEEWACLGPIQRALYWDVM |
| | HUMAN | LENYGNVTSLEWETMTENEEVTSKPSSSQR |
| 392 | ZN717_ | LETYNSLVSLQELVSFEEVAVHFTWEEWQDLDDAQRTLYRDVMLETYSSL |
| | HUMAN | VSLGHCITKPEMIFKLEQGAEPWIVEETPN |
| 393 | ZN669_ | RHFRRPEPCREPLASPIQDSVAFEDVAVNFTQEEWALLDSSQKNLYREVMQ |
| | HUMAN | ETCRNLASVGSQWKDQNIEDHFEKPGKDI |
| 394 | ZN256_ | AAAELTAPAQGIVTFEDVAVYFSWKEWGLLDEAQKCLYHDVMLENLTLTT |
| | HUMAN | SLGGSGAGDEEAPYQQSTSPQRVSQVRIPK |
| 395 | ZN251_ | AATFQLPGHQEMPLTFQDVAVYFSQAEGRQLGPQQRALYRDVMLENYGN |
| | HUMAN | VASLGFPVPKPELISQLEQGKELWVLNLLGA |
| 396 | CBX4_ | RSEAGEPPSSLQVKPETPASAAVAVAAAAAPTTTAEKPPAEAQDEPAESLSE |
| | HUMAN | FKPFFGNIIITDVTANCLTVTFKEYVTV |
| 397 | PCGF2_ | HRTTRIKITELNPHLMCALCGGYFIDATTIVECLHSFCKTCIVRYLETNKYCP |
| | HUMAN | MCDVQVHKTRPLLSIRSDKTLQDIVYK |
| 398 | CDY2_ | ASQEFEVEAIVDKRQDKNGNTQYLVRWKGYDKQDDTWEPEQHLMNCEKC |
| | HUMAN | VHDFNRRQTEKQKKLTWTTTSRIFSNNARRR |
| 399 | CDYL2_ | ASGDLYEVERIVDKRKNKKGKWEYLIRWKGYGSTEDTWEPEHHLLHCEEF |
| | HUMAN | IDEFNGLHMSKDKRIKSGKQSSTSKLLRDS |
| 400 | HERC2_ | TLIRKADLENHNKDGGFWTVIDGKVYDIKDFQTQSLTGNSILAQFAGEDPV |
| | HUMAN | VALEAALQFEDTRESMHAFCVGQYLEPDQ |
| 401 | ZN562_ | EKTKIGTMVEDHRSNSYQDSVTFDDVAVEFTPEEWALLDTTQKYLYRDVM |
| | HUMAN | LENYMNLASVDFFFCLTSEWEIQPRTKRSS |
| 402 | ZN461_ | AHELVMFRDVAIDVSQEEWECLNPAQRNLYKEVMLENYSNLVSLGLSVSK |
| | HUMAN | PAVISSLEQGKEPWMVVREETGRWCPGTWK |
| 403 | Z324A_ | AFEDVAVYFSQEEWGLLDTAQRALYRRVMLDNFALVASLGLSTSRPRVVI |
| | HUMAN | QLERGEEPWVPSGTDTTLSRTTYRRRNPGS |
| 404 | ZN766_ | AQLRRGHLTFRDVAIEFSQEEWKCLDPVQKALYRDVMLENYRNLVSLGICL |
| | HUMAN | PDLSIISMMKQRTEPWTVENEMKVAKNPD |
| 405 | ID2_ | SDHSLGISRSKTPVDDPMSLLYNMNDCYSKLKELVPSIPQNKKVSKMEILQH |
| | HUMAN | VIDYILDLQIALDSHPTIVSLHHQRPGQ |
| 406 | TOX_ | KDPNEPQKPVSAYALFFRDTQAAIKGQNPNATFGEVSKIVASMWDGLGEE |
| | HUMAN | QKQVYKKKTEAAKKEYLKQLAAYRASLVSK |
| 407 | ZN274_ | QEEKQEDAAICPVTVLPEEPVTFQDVAVDFSREEWGLLGPTQRTEYRDVML |
| | HUMAN | ETFGHLVSVGWETTLENKELAPNSDIPEE |
| 408 | SCMH1_ | DASRLSGRDPSSWTVEDVMQFVREADPQLGPHADLFRKHEIDGKALLLLRS |
| | HUMAN | DMMMKYMGLKLGPALKLSYHIDRLKQGKF |
| 409 | ZN214_ | AVTFEDVTIIFTWEEWKFLDSSQKRLYREVMWENYTNVMSVENWNESYKS |
| | HUMAN | QEEKFRYLEYENFSYWQGWWNAGAQMYENQ |
| 410 | CBX7_ | ELSAIGEQVFAVESIRKKRVRKGKVEYLVKWKGWPPKYSTWEPEEHILDPR |
| | HUMAN | LVMAYEEKEERDRASGYRKRGPKPKRLLL |
| 411 | ID1_ | GGAGARLPALLDEQQVNVLLYDMNGCYSRLKELVPTLPQNRKVSKVEILQ |
| | HUMAN | HVIDYIRDLQLELNSESEVGTPGGRGLPVR |
| 412 | CREM_ | VVMAASPGSLHSPQQLAEEATRKRELRLMKNREAAKECRRRKKEYVKCLE |
| | HUMAN | SRVAVLEVQNKKLIEELETLKDICSPKTDY |
| 413 | SCX_ | GGGPGGRPGREPRQRHTANARERDRTNSVNTAFTALRTLIPTEPADRKLSKI |
| | HUMAN | ETLRLASSYISHLGNVLLAGEACGDGQP |
| 414 | ASCL1_ | SGFGYSLPQQQPAAVARRNERERNRVKLVNLGFATLREHVPNGAANKKMS |
| | HUMAN | KVETLRSAVEYIRALQQLLDEHDAVSAAFQ |
| 415 | ZN764_ | APLPPRDPNGAGPEWREPGAVSFADVAVYFCREEWGCLRPAQRALYRDV |
| | HUMAN | MRETYGHLSALGIGGNKPALISWVEEEAELW |
| 416 | SCML2_ | KQGFSKDPSTWSVDEVIQFMKHTDPQISGPLADLFRQHEIDGKALFLLKSDV |
| | HUMAN | MMKYMGLKLGPALKLCYYIEKLKEGKYS |
| 417 | TWST1_ | SGGGSPQSYEELQTQRVMANVRERQRTQSLNEAFAALRKIIPTLPSDKLSKI |
| | HUMAN | QTLKLAARYIDFLYQVLQSDELDSKMAS |
| 418 | CREB1_ | IAPGVVMASSPALPTQPAEEAARKREVRLMKNREAARECRRKKKEYVKCL |
| | HUMAN | ENRVAVLENQNKTLIEELKALKDLYCHKSD |
| 419 | TERF1_ | SRIPVSKSQPVTPEKHRARKRQAWLWEEDKNLRSGVRKYGEGNWSKILLH |
| | HUMAN | YKFNNRTSVMLKDRWRTMKKLKLISSDSED |
| 420 | ID3_ | SLAIARGRGKGPAAEEPLSLLDDMNHCYSRLRELVPGVPRGTQLSQVEILQR |
| | HUMAN | VIDYILDLQVVLAEPAPGPPDGPHLPIQ |
| 421 | CBX8_ | GSGPPSSGGGLYRDMGAQGGRPSLIARIPVARILGDPEEESWSPSLTNLEKV |
| | HUMAN | VVTDVTSNFLTVTIKESNTDQGFFKEKR |
| 422 | CBX4_ | ELPAVGEHVFAVESIEKKRIRKGRVEYLVKWRGWSPKYNTWEPEENILDPR |
| | HUMAN | LLIAFQNRERQEQLMGYRKRGPKPKPLVV |
| 423 | GSX1_ | VDSSSNQLPSSKRMRTAFTSTQLLELEREFASNMYLSRLRRIEIATYLNLSEK |
| | HUMAN | QVKIWFQNRRVKHKKEGKGSNHRGGGG |
| 424 | NKX22_ | TPGGGGDAGKKRKRRVLFSKAQTYELERRFRQQRYLSAPEREHLASLIRLT |
| | HUMAN | PTQVKIWFQNHRYKMKRARAEKGMEVTPL |
| 425 | ATF1_ | QTVVMTSPVTLTSQTTKTDDPQLKREIRLMKNREAARECRRKKKEYVKCL |
| | HUMAN | ENRVAVLENQNKTLIEELKTLKDLYSNKSV |
| 426 | TWST2_ | KGSPSAQSFEELQSQRILANVRERQRTQSLNEAFAALRKIIPTLPSDKLSKIQT |
| | HUMAN | LKLAARYIDFLYQVLQSDEMDNKMTS |
| 427 | ZNF17_ | NLTEDYMVFEDVAIHFSQEEWGILNDVQRHLHSDVMLENFALLSSVGCWH |
| | HUMAN | GAKDEEAPSKQCVSVGVSQVTTLKPALSTQ |
| 428 | TOX3_ | KDPNEPQKPVSAYALFFRDTQAAIKGQNPNATFGEVSKIVASMWDSLGEEQ |
| | HUMAN | KQVYKRKTEAAKKEYLKALAAYRASLVSK |
| 429 | TOX4_ | KDPNEPQKPVSAYALFFRDTQAAIKGQNPNATFGEVSKIVASMWDSLGEEQ |
| | HUMAN | KQVYKRKTEAAKKEYLKALAAYKDNQECQ |
| 430 | ZMYM3_ | LDGSTWDFCSEDCKSKYLLWYCKAARCHACKRQGKLLETIHWRGQIRHFC |
| | HUMAN | NQQCLLRFYSQQNQPNLDTQSGPESLLNSQ |
| 431 | I2BP1_ | ASVQASRRQWCYLCDLPKMPWAMVWDFSEAVCRGCVNFEGADRIELLID |
| | HUMAN | AARQLKRSHVLPEGRSPGPPALKHPATKDLA |
| 432 | RHXF1_ | MEGPQPENMQPRTRRTKFTLLQVEELESVFRHTQYPDVPTRRELAENLGVT |
| | HUMAN | EDKVRVWFKNKRARCRRHQRELMLANELR |
| 433 | SSX2_ | PKIMPKKPAEEGNDSEEVPEASGPQNDGKELCPPGKPTTSEKIHERSGPKRG |
| | HUMAN | EHAWTHRLRERKQLVIYEEISDPEEDDE |
| 434 | I2BPL_ | SAAQVSSSRRQSCYLCDLPRMPWAMIWDFSEPVCRGCVNYEGADRIEFVIE |
| | HUMAN | TARQLKRAHGCFQDGRSPGPPPPVGVKTV |
| 435 | ZN680_ | PGPPGSLEMGPLTFRDVAIEFSLEEWQCLDTAQRNLYRKVMFENYRNLVFL |
| | HUMAN | GIAVSKPHLITCLEQGKEPWNRKRQEMVA |
| 436 | CBX1_ | NKKKVEEVLEEEEEEYVVEKVLDRRVVKGKVEYLLKWKGFSDEDNTWEP |
| | HUMAN | EENLDCPDLIAEFLQSQKTAHETDKSEGGKR |
| 437 | TRI68_ | LANVVEKVRLLRLHPGMGLKGDLCERHGEKLKMFCKEDVLIMCEACSQSP |
| | HUMAN | EHEAHSVVPMEDVAWEYKWELHEALEHLKK |
| 438 | HXA13_ | VVSHPSDASSYRRGRKKRVPYTKVQLKELEREYATNKFITKDKRRRISATT |
| | HUMAN | NLSERQVTIWFQNRRVKEKKVINKLKTTS |
| 439 | PHC3_ | ENSDLLPVAQTEPSIWTVDDVWAFIHSLPGCQDIADEFRAQEIDGQALLLLK |
| | HUMAN | EDHLMSAMNIKLGPALKICARINSLKES |
| 440 | TCF24_ | AGPGGGSRSGSGRPAAANAARERSRVQTLRHAFLELQRTLPSVPPDTKLSK |
| | HUMAN | LDVLLLATTYIAHLTRSLQDDAEAPADAG |
| 441 | CBX3_ | QNGKSKKVEEAEPEEFVVEKVLDRRVVNGKVEYFLKWKGFTDADNTWEP |
| | HUMAN | EENLDCPELIEAFLNSQKAGKEKDGTKRKSL |
| 442 | HXB13_ | QHPPDACAFRRGRKKRIPYSKGQLRELEREYAANKFITKDKRRKISAATSLS |
| | HUMAN | ERQITIWFQNRRVKEKKVLAKVKNSATP |
| 443 | HEY1_ | SMSPTTSSQILARKRRRGIIEKRRRDRINNSLSELRRLVPSAFEKQGSAKLEK |
| | HUMAN | AEILQMTVDHLKMLHTAGGKGYFDAHA |
| 444 | PHC2_ | LVGMGHHFLPSEPTKWNVEDVYEFIRSLPGCQEIAEEFRAQEIDGQALLLLK |
| | HUMAN | EDHLMSAMNIKLGPALKIYARISMLKDS |
| 445 | ZNF81_ | PANEDAPQPGEHGSACEVSVSFEDVTVDFSREEWQQLDSTQRRLYQDVML |
| | HUMAN | ENYSHLLSVGFEVPKPEVIFKLEQGEGPWT |
| 446 | FIGLA_ | GYSSTENLQLVLERRRVANAKERERIKNLNRGFARLKALVPFLPQSRKPSK |
| | HUMAN | VDILKGATEYIQVLSDLLEGAKDSKKQDP |
| 447 | SAM11_ | EEAPAPEDVTKWTVDDVCSFVGGLSGCGEYTRVFREQGIDGETLPLLTEEH |
| | HUMAN | LLTNMGLKLGPALKIRAQVARRLGRVFYV |
| 448 | KMT2B_ | GGTLAHTPRRSLPSHHGKKMRMARCGHCRGCLRVQDCGSCVNCLDKPKF |
| | HUMAN | GGPNTKKQCCVYRKCDKIEARKMERLAKKGR |
| 449 | HEY2_ | LNSPTTTSQIMARKKRRGIIEKRRRDRINNSLSELRRLVPTAFEKQGSAKLEK |
| | HUMAN | AEILQMTVDHLKMLQATGGKGYFDAHA |
| 450 | JDP2_ | QPVKSELDEEEERRKRRREKNKVAAARCRNKKKERTEFLQRESERLELMN |
| | HUMAN | AELKTQIEELKQERQQLILMLNRHRPTCIV |
| 451 | HXC13_ | LQPEVSSYRRGRKKRVPYTKVQLKELEKEYAASKFITKEKRRRISATTNLSE |
| | HUMAN | RQVTIWFQNRRVKEKKVVSKSKAPHLHS |
| 452 | ASCL4_ | LPVPLDSAFEPAFLRKRNERERQRVRCVNEGYARLRDHLPRELADKRLSKV |
| | HUMAN | ETLRAAIDYIKHLQELLERQAWGLEGAAG |
| 453 | HHEX_ | SPFLQRPLHKRKGGQVRFSNDQTIELEKKFETQKYLSPPERKRLAKMLQLSE |
| | HUMAN | RQVKTWFQNRRAKWRRLKQENPQSNKKE |
| 454 | HERC2_ | IAIATGSLHCVCCTEDGEVYTWGDNDEGQLGDGTTNAIQRPRLVAALQGK |
| | HUMAN | KVNRVACGSAHTLAWSTSKPASAGKLPAQV |
| 455 | GSX2_ | GGSDASQVPNGKRMRTAFTSTQLLELEREFSSNMYLSRLRRIEIATYLNLSE |
| | HUMAN | KQVKIWFQNRRVKHKKEGKGTQRNSHAG |
| 456 | BIN1_ | RLDLPPGFMFKVQAQHDYTATDTDELQLKAGDVVLVIPFQNPEEQDEGWL |
| | HUMAN | MGVKESDWNQHKELEKCRGVFPENFTERVP |
| 457 | ETV7_ | GICKLPGRLRIQPALWSREDVLHWLRWAEQEYSLPCTAEHGFEMNGRALCI |
| | HUMAN | LTKDDFRHRAPSSGDVLYELLQYIKTQRR |
| 458 | ASCL3_ | PNYRGCEYSYGPAFTRKRNERERQRVKCVNEGYAQLRHHLPEEYLEKRLS |
| | HUMAN | KVETLRAAIKYINYLQSLLYPDKAETKNNP |
| 459 | PHC1_ | LHGINPVFLSSNPSRWSVEEVYEFIASLQGCQEIAEEFRSQEIDGQALLLLKE |
| | HUMAN | EHLMSAMNIKLGPALKICAKINVLKET |
| 460 | OTP_ | QAGQQQGQQKQKRHRTRFTPAQLNELERSFAKTHYPDIFMREELALRIGLT |
| | AHUMANN | ESRVQVWFQNRRAKWKKRKKTTNVFRAPG |
| 461 | I2BP2_ | AAAVAVAAASRRQSCYLCDLPRMPWAMIWDFTEPVCRGCVNYEGADRVE |
| | HUMAN | FVIETARQLKRAHGCFPEGRSPPGAAASAAA |
| 462 | VGLL2_ | FSSQTPASIKEEEGSPEKERPPEAEYINSRCVLFTYFQGDISSVVDEHFSRALS |
| | HUMAN | QPSSYSPSCTSSKAPRSSGPWRDCSF |
| 463 | HXA11_ | DKAGGSSGQRTRKKRCPYTKYQIRELEREFFFSVYINKEKRLQLSRMLNLT |
| | HUMAN | DRQVKIWFQNRRMKEKKINRDRLQYYSAN |
| 464 | PDLI4_ | GAPLSGLQGLPECTRCGHGIVGTIVKARDKLYHPECFMCSDCGLNLKQRGY |
| | HUMAN | FFLDERLYCESHAKARVKPPEGYDVVAVY |
| 465 | ASCL2_ | RRPATAETGGGAAAVARRNERERNRVKLVNLGFQALRQHVPHGGASKKL |
| | HUMAN | SKVETLRSAVEYIRALQRLLAEHDAVRNALA |
| 466 | CDX4_ | TVQVTGKTRTKEKYRVVYTDHQRLELEKEFHCNRYITIQRKSELAVNLGLS |
| | HUMAN | ERQVKIWFQNRRAKERKMIKKKISQFENS |
| 467 | ZN860_ | EEAAQKRKEKEPGMALPQGHLTFRDVAIEFSLEEWKCLDPTQRALYRAMM |
| | HUMAN | LENYRNLHSVDISSKCMMKKFSSTAQGNTE |
| 468 | LMBL4_ | DIRASQVARWTVDEVAEFVQSLLGCEEHAKCFKKEQIDGKAFLLLTQTDIV |
| | HUMAN | KVMKIKLGPALKIYNSILMFRHSQELPEE |
| 469 | PDIP3_ | LSPLEGTKMTVNNLHPRVTEEDIVELFCVCGALKRARLVHPGVAEVVFVKK |
| | HUMAN | DDAITAYKKYNNRCLDGQPMKCNLHMNGN |
| 470 | NKX25_ | DNAERPRARRRRKPRVLFSQAQVYELERRFKQQRYLSAPERDQLASVLKLT |
| | HUMAN | STQVKIWFQNRRYKCKRQRQDQTLELVGL |
| 471 | CEBPB_ | SQVKSKAKKTVDKHSDEYKIRRERNNIAVRKSRDKAKMRNLETQHKVLEL |
| | HUMAN | TAENERLQKKVEQLSRELSTLRNLFKQLPE |
| 472 | ISL1_ | KRDYIRLYGIKCAKCSIGFSKNDFVMRARSKVYHIECFRCVACSRQLIPGDE |
| | HUMAN | FALREDGLFCRADHDVVERASLGAGDPL |
| 473 | CDX2_ | SLGSQVKTRTKDKYRVVYTDHQRLELEKEFHYSRYITIRRKAELAATLGLS |
| | HUMAN | ERQVKIWFQNRRAKERKINKKKLQQQQQQ |
| 474 | PROP1_ | QGGQRGRPHSRRRHRTTFSPVQLEQLESAFGRNQYPDIWARESLARDTGLS |
| | HUMAN | EARIQVWFQNRRAKQRKQERSLLQPLAHL |
| 475 | SIN3B_ | DALTYLDQVKIRFGSDPATYNGFLEIMKEFKSQSIDTPGVIRRVSQLFHEHPD |
| | HUMAN | LIVGFNAFLPLGYRIDIPKNGKLNIQS |
| 476 | SMBT1_ | RLHLDSNPLKWSVADVVRFIRSTDCAPLARIFLDQEIDGQALLLLTLPTVQE |
| | HUMAN | CMDLKLGPAIKLCHHIERIKFAFYEQFA |
| 477 | HXC11_ | AKGAAPNAPRTRKKRCPYSKFQIRELEREFFFNVYINKEKRLQLSRMLNLTD |
| | HUMAN | RQVKIWFQNRRMKEKKLSRDRLQYFSGN |
| 478 | HXC10_ | TTGNWLTAKSGRKKRCPYTKHQTLELEKEFLFNMYLTRERRLEISKTINLTD |
| | HUMAN | RQVKIWFQNRRMKLKKMNRENRIRELTS |
| 479 | PRS6A_ | YLVSNVIELLDVDPNDQEEDGANIDLDSQRKGKCAVIKTSTRQTYFLPVIGL |
| | HUMAN | VDAEKLKPGDLVGVNKDSYLILETLPTE |
| 480 | VSX1_ | KASPTLGKRKKRRHRTVFTAHQLEELEKAFSEAHYPDVYAREMLAVKTEL |
| | HUMAN | PEDRIQVWFQNRRAKWRKREKRWGGSSVMA |
| 481 | NKX23_ | EESERPKPRSRRKPRVLFSQAQVFELERRFKQQRYLSAPEREHLASSLKLTST |
| | HUMAN | QVKIWFQNRRYKCKRQRQDKSLELGAH |
| 482 | MTG16_ | VVPGSRQEEVIDHKLTEREWAEEWKHLNNLLNCIMDMVEKTRRSLTVLRR |
| | HUMAN | CQEADREELNHWARRYSDAEDTKKGPAPAA |
| 483 | HMX3_ | ESPEKKPACRKKKTRTVFSRSQVFQLESTFDMKRYLSSSERAGLAASLHLTE |
| | HUMAN | TQVKIWFQNRRNKWKRQLAAELEAANLS |
| 484 | HMX1_ | RGGVGVGGGRKKKTRTVFSRSQVFQLESTFDLKRYLSSAERAGLAASLQLT |
| | HUMAN | ETQVKIWFQNRRNKWKRQLAAELEAASLS |
| 485 | KIF22_ | ELLAHGRQKILDLLNEGSARDLRSLQRIGPKKAQLIVGWRELHGPFSQVEDL |
| | HUMAN | ERVEGITGKQMESFLKANILGLAAGQRC |
| 486 | CSTF2_ | ESPYGETISPEDAPESISKAVASLPPEQMFELMKQMKLCVQNSPQEARNMLL |
| | HUMAN | QNPQLAYALLQAQVVMRIVDPEIALKIL |
| 487 | CEBPE_ | AGPLHKGKKAVNKDSLEYRLRRERNNIAVRKSRDKAKRRILETQQKVLEY |
| | HUMAN | MAENERLRSRVEQLTQELDTLRNLFRQIPE |
| 488 | DLX2_ | IRIVNGKPKKVRKPRTIYSSFQLAALQRRFQKTQYLALPERAELAASLGLTQ |
| | HUMAN | TQVKIWFQNRRSKFKKMWKSGEIPSEQH |
| 489 | ZMYM3_ | TVYQFCSPSCWTKFQRTSPEGGIHLSCHYCHSLFSGKPEVLDWQDQVFQFC |
| | HUMAN | CRDCCEDFKRLRGVVSQCEHCRQEKLLHE |
| 490 | PPARG_ | TMVDTEMPFWPTNFGISSVDLSVMEDHSHSFDIKPFTTVDFSSISTPHYEDIP |
| | HUMAN | FTRTDPVVADYKYDLKLQEYQSAIKVE |
| 491 | PRIC1_ | GRHHAELLKPRCSACDEIIFADECTEAEGRHWHMKHFCCLECETVLGGQRY |
| | HUMAN | IMKDGRPFCCGCFESLYAEYCETCGEHIG |
| 492 | UNC4_ | DPDKESPGCKRRRTRTNFTGWQLEELEKAFNESHYPDVFMREALALRLDL |
| | HUMAN | VESRVQVWFQNRRAKWRKKENTKKGPGRPA |
| 493 | BARX2_ | TEQPTPRQKKPRRSRTIFTELQLMGLEKKFQKQKYLSTPDRLDLAQSLGLTQ |
| | HUMAN | LQVKTWYQNRRMKWKKMVLKGGQEAPTK |
| 494 | ALX3_ | SMELAKNKSKKRRNRTTFSTFQLEELEKVFQKTHYPDVYAREQLALRTDLT |
| | HUMAN | EARVQVWFQNRRAKWRKRERYGKIQEGRN |
| 495 | TCF15_ | GGGGGAGPVVVVRQRQAANARERDRTQSVNTAFTALRTLIPTEPVDRKLS |
| | HUMAN | KIETVRLASSYIAHLANVLLLGDSADDGQP |
| 496 | TERA_ | IDDTVEGITGNLFEVYLKPYFLEAYRPIRKGDIFLVRGGMRAVEFKVVETDP |
| | HUMAN | SPYCIVAPDTVIHCEGEPIKREDEEESL |
| 497 | VSX2_ | SALNQTKKRKKRRHRTIFTSYQLEELEKAFNEAHYPDVYAREMLAMKTEL |
| | HUMAN | PEDRIQVWFQNRRAKWRKREKCWGRSSVMA |
| 498 | HXD12_ | DGLPWGAAPGRARKKRKPYTKQQIAELENEFLVNEFINRQKRKELSNRLNL |
| | HUMAN | SDQQVKIWFQNRRMKKKRVVLREQALALY |
| 499 | CDX1_ | GGGGSGKTRTKDKYRVVYTDHQRLELEKEFHYSRYITIRRKSELAANLGLT |
| | HUMAN | ERQVKIWFQNRRAKERKVNKKKQQQQQPP |
| 500 | TCF23_ | TRAGGLALGRSEASPENAARERSRVRTLRQAFLALQAALPAVPPDTKLSKL |
| | HUMAN | DVLVLAASYIAHLTRTLGHELPGPAWPPF |
| 501 | ALX1_ | KCDSNVSSSKKRRHRTTFTSLQLEELEKVFQKTHYPDVYVREQLALRTELT |
| | HUMAN | EARVQVWFQNRRAKWRKRERYGQIQQAKS |
| 502 | HXA10_ | NAANWLTAKSGRKKRCPYTKHQTLELEKEFLFNMYLTRERRLEISRSVHLT |
| | HUMAN | DRQVKIWFQNRRMKLKKMNRENRIRELTA |
| 503 | RX_ | LSEEEQPKKKHRRNRTTFTTYQLHELERAFEKSHYPDVYSREELAGKVNLP |
| | HUMAN | EVRVQVWFQNRRAKWRRQEKLEVSSMKLQ |
| 504 | CXXC5_ | HMAGLAEYPMQGELASAISSGKKKRKRCGMCAPCRRRINCEQCSSCRNRK |
| | HUMAN | TGHQICKFRKCEELKKKPSAALEKVMLPTG |
| 505 | SCML1_ | SITKHPSTWSVEAVVLFLKQTDPLALCPLVDLFRSHEIDGKALLLLTSDVLL |
| | HUMAN | KHLGVKLGTAVKLCYYIDRLKQGKCFEN |
| 506 | NFIL3_ | ACRRKREFIPDEKKDAMYWEKRRKNNEAAKRSREKRRLNDLVLENKLIAL |
| | HUMAN | GEENATLKAELLSLKLKFGLISSTAYAQEI |
| 507 | DLX6_ | EIRFNGKGKKIRKPRTIYSSLQLQALNHRFQQTQYLALPERAELAASLGLTQ |
| | HUMAN | TQVKIWFQNKRSKFKKLLKQGSNPHESD |
| 508 | MTG8_ | GLHGTRQEEMIDHRLTDREWAEEWKHLDHLLNCIMDMVEKTRRSLTVLRR |
| | HUMAN | CQEADREELNYWIRRYSDAEDLKKGGGSSS |
| 509 | CBX8_ | ELSAVGERVFAAEALLKRRIRKGRMEYLVKWKGWSQKYSTWEPEENILDA |
| | HUMAN | RLLAAFEEREREMELYGPKKRGPKPKTFLL |
| 510 | CEBPD_ | AREKSAGKRGPDRGSPEYRQRRERNNIAVRKSRDKAKRRNQEMQQKLVEL |
| | HUMAN | SAENEKLHQRVEQLTRDLAGLRQFFKQLPS |
| 511 | SEC13_ | SGGCDNLIKLWKEEEDGQWKEEQKLEAHSDWVRDVAWAPSIGLPTSTIAS |
| | HUMAN | CSQDGRVFIWTCDDASSNTWSPKLLHKFND |
| 512 | FIP1_ | VKGVDLDAPGSINGVPLLEVDLDSFEDKPWRKPGADLSDYFNYGFNEDTW |
| | HUMAN | KAYCEKQKRIRMGLEVIPVTSTTNKITAED |
| 513 | ALX4_ | KADSESNKGKKRRNRTTFTSYQLEELEKVFQKTHYPDVYAREQLAMRTDL |
| | HUMAN | TEARVQVWFQNRRAKWRKRERFGQMQQVRT |
| 514 | LHX3_ | TAKQREAEATAKRPRTTITAKQLETLKSAYNTSPKPARHVREQLSSETGLD |
| | HUMAN | MRVVQVWFQNRRAKEKRLKKDAGRQRWGQ |
| 515 | PRIC2_ | GRHHAECLKPRCAACDEIIFADECTEAEGRHWHMKHFCCFECETVLGGQR |
| | HUMAN | YIMKEGRPYCCHCFESLYAEYCDTCAQHIG |
| 516 | MAGI3_ | IIGGDRPDEFLQVKNVLKDGPAAQDGKIAPGDVIVDINGNCVLGHTHADVV |
| | HUMAN | QMFQLVPVNQYVNLTLCRGYPLPDDSEDP |
| 517 | NELL1_ | CCPECDTRVTSQCLDQNGHKLYRSGDNWTHSCQQCRCLEGEVDCWPLTCP |
| | HUMAN | NLSCEYTAILEGECCPRCVSDPCLADNITY |
| 518 | PRRX1_ | LNSEEKKKRKQRRNRTTFNSSQLQALERVFERTHYPDAFVREDLARRVNLT |
| | HUMAN | EARVQVWFQNRRAKFRRNERAMLANKNAS |
| 519 | MTG8R_ | GLNGGYQDELVDHRLTEREWADEWKHLDHALNCIMEMVEKTRRSMAVL |
| | HUMAN | RRCQESDREELNYWKRRYNENTELRKTGTELV |
| 520 | RAX2_ | GPGEEAPKKKHRRNRTTFTTYQLHQLERAFEASHYPDVYSREELAAKVHLP |
| | HUMAN | EVRVQVWFQNRRAKWRRQERLESGSGAVA |
| 521 | DLX3_ | VRMVNGKPKKVRKPRTIYSSYQLAALQRRFQKAQYLALPERAELAAQLGL |
| | HUMAN | TQTQVKIWFQNRRSKFKKLYKNGEVPLEHS |
| 522 | DLX1_ | EVRFNGKGKKIRKPRTIYSSLQLQALNRRFQQTQYLALPERAELAASLGLTQ |
| | HUMAN | TQVKIWFQNKRSKFKKLMKQGGAALEGS |
| 523 | NKX26_ | GRSEQPKARQRRKPRVLFSQAQVLALERRFKQQRYLSAPEREHLASALQLT |
| | HUMAN | STQVKIWFQNRRYKCKRQRQDKSLELAGH |
| 524 | NAB1_ | LPRTLGELQLYRILQKANLLSYFDAFIQQGGDDVQQLCEAGEEEFLEIMALV |
| | HUMAN | GMASKPLHVRRLQKALRDWVTNPGLFNQ |
| 525 | SAMD7_ | NLSLDEDIQKWTVDDVHSFIRSLPGCSDYAQVFKDHAIDGETLPLLTEEHLR |
| | HUMAN | GTMGLKLGPALKIQSQVSQHVGSMFYKK |
| 526 | PITX3_ | SPEDGSLKKKQRRQRTHFTSQQLQELEATFQRNRYPDMSTREEIAVWTNLT |
| | HUMAN | EARVRVWFKNRRAKWRKRERSQQAELCKG |
| 527 | WDR5_ | SNLLVSASDDKTLKIWDVSSGKCLKTLKGHSNYVFCCNFNPQSNLIVSGSFD |
| | HUMAN | ESVRIWDVKTGKCLKTLPAHSDPVSAVH |
| 528 | MEOX2_ | GNYKSEVNSKPRKERTAFTKEQIRELEAEFAHHNYLTRLRRYEIAVNLDLTE |
| | HUMAN | RQVKVWFQNRRMKWKRVKGGQQGAAARE |
| 529 | NAB2_ | LPRTLGELQLYRVLQRANLLSYYETFIQQGGDDVQQLCEAGEEEFLEIMAL |
| | HUMAN | VGMATKPLHVRRLQKALREWATNPGLFSQ |
| 530 | DHX8_ | PEEPTIGDIYNGKVTSIMQFGCFVQLEGLRKRWEGLVHISELRREGRVANVA |
| | HUMAN | DVVSKGQRVKVKVLSFTGTKTSLSMKDV |
| 531 | FOXA2_ | YAFNHPFSINNLMSSEQQHHHSHHHHQPHKMDLKAYEQVMHYPGYGSPM |
| | HUMAN | PGSLAMGPVTNKTGLDASPLAADTSYYQGVY |
| 532 | CBX6_ | TAAAGPAPPTAPEPAGASSEPEAGDWRPEMSPCSNVVVTDVTSNLLTVTIK |
| | HUMAN | EFCNPEDFEKVAAGVAGAAGGGGSIGASK |
| 533 | EMX2_ | FLLHNALARKPKRIRTAFSPSQLLRLEHAFEKNHYVVGAERKQLAHSLSLTE |
| | HUMAN | TQVKVWFQNRRTKFKRQKLEEEGSDSQQ |
| 534 | CPSF6_ | KRIALYIGNLTWWTTDEDLTEAVHSLGVNDILEIKFFENRANGQSKGFALV |
| | HUMAN | GVGSEASSKKLMDLLPKRELHGQNPVVTP |
| 535 | HXC12_ | SGAPWYPINSRSRKKRKPYSKLQLAELEGEFLVNEFITRQRRRELSDRLNLS |
| | HUMAN | DQQVKIWFQNRRMKKKRLLLREQALSFF |
| 536 | KDM4B_ | SDNLYPESITSRDCVQLGPPSEGELVELRWTDGNLYKAKFISSVTSHIYQVEF |
| | HUMAN | EDGSQLTVKRGDIFTLEEELPKRVRSR |
| 537 | LMBL3_ | GIPASKVSKWSTDEVSEFIQSLPGCEEHGKVFKDEQIDGEAFLLMTQTDIVKI |
| | HUMAN | MSIKLGPALKIFNSILMFKAAEKNSHN |
| 538 | PHX2A_ | EPSGLHEKRKQRRIRTTFTSAQLKELERVFAETHYPDIYTREELALKIDLTEA |
| | HUMAN | RVQVWFQNRRAKFRKQERAASAKGAAG |
| 539 | EMX1_ | LLLHGPFARKPKRIRTAFSPSQLLRLERAFEKNHYVVGAERKQLAGSLSLSE |
| | HUMAN | TQVKVWFQNRRTKYKRQKLEEEGPESEQ |
| 540 | NC2B_ | SSGNDDDLTIPRAAINKMIKETLPNVRVANDARELVVNCCTEFIHLISSEANE |
| | HUMAN | ICNKSEKKTISPEHVIQALESLGFGSY |
| 541 | DLX4_ | ERRPQAPAKKLRKPRTIYSSLQLQHLNQRFQHTQYLALPERAQLAAQLGLT |
| | HUMAN | QTQVKIWFQNKRSKYKKLLKQNSGGQEGD |
| 542 | SRY_ | NVQDRVKRPMNAFIVWSRDQRRKMALENPRMRNSEISKQLGYQWKMLTE |
| | HUMAN | AEKWPFFQEAQKLQAMHREKYPNYKYRPRRK |
| 543 | ZN777_ | EITRLAVWAAVQAVERKLEAQAMRLLTLEGRTGTNEKKIADCEKTAVEFA |
| | HUMAN | NHLESKWVVLGTLLQEYGLLQRRLENMENL |
| 544 | NELL1_ | CEKDIDECSEGIIECHNHSRCVNLPGWYHCECRSGFHDDGTYSLSGESCIDID |
| | HUMAN | ECALRTHTCWNDSACINLAGGFDCLCP |
| 545 | ZN398_ | AAISLWTVVAAVQAIERKVEIHSRRLLHLEGRTGTAEKKLASCEKTVTELG |
| | HUMAN | NQLEGKWAVLGTLLQEYGLLQRRLENLEN |
| 546 | GATA3_ | GQNRPLIKPKRRLSAARRAGTSCANCQTTTTTLWRRNANGDPVCNACGLY |
| | HUMAN | YKLHNINRPLTMKKEGIQTRNRKMSSKSKK |
| 547 | BSH_ | HAELPGKHCRRRKARTVFSDSQLSGLEKRFEIQRYLSTPERVELATALSLSE |
| | HUMAN | TQVKTWFQNRRMKHKKQLRKSQDEPKAP |
| 548 | SF3B4_ | QDATVYVGGLDEKVSEPLLWELFLQAGPVVNTHMPKDRVTGQHQGYGFV |
| | HUMAN | EFLSEEDADYAIKIMNMIKLYGKPIRVNKAS |
| 549 | TEAD1_ | PIDNDAEGVWSPDIEQSFQEALAIYPPCGRRKIILSDEGKMYGRNELIARYIK |
| | HUMAN | LRTGKTRTRKQVSSHIQVLARRKSRDF |
| 550 | TEAD3_ | GLDNDAEGVWSPDIEQSFQEALAIYPPCGRRKIILSDEGKMYGRNELIARYI |
| | HUMAN | KLRTGKTRTRKQVSSHIQVLARKKVREY |
| 551 | RGAP1_ | DSVGTPQSNGGMRLHDFVSKTVIKPESCVPCGKRIKFGKLSLKCRDCRVVS |
| | HUMAN | HPECRDRCPLPCIPTLIGTPVKIGEGMLA |
| 552 | PHF1_ | SAPHSMTASSSSVSSPSPGLPRRSAPPSPLCRSLSPGTGGGVRGGVGYLSRGD |
| | HUMAN | PVRVLARRVRPDGSVQYLVEWGGGGIF |
| 553 | FOXA1_ | GDPHYSFNHPFSINNLMSSSEQQHKLDFKAYEQALQYSPYGSTLPASLPLGS |
| | HUMAN | ASVTTRSPIEPSALEPAYYQGVYSRPVL |
| 554 | GATA2_ | GQNRPLIKPKRRLSAARRAGTCCANCQTTTTTLWRRNANGDPVCNACGLY |
| | HUMAN | YKLHNVNRPLTMKKEGIQTRNRKMSNKSKK |
| 555 | FOXO3_ | DSLSGSSLYSTSANLPVMGHEKFPSDLDLDMFNGSLECDMESIIRSELMDAD |
| | HUMAN | GLDFNFDSLISTQNVVGLNVGNFTGAKQ |
| 556 | ZN212_ | TEISLWTVVAAIQAVEKKMESQAARLQSLEGRTGTAEKKLADCEKMAVEF |
| | HUMAN | GNQLEGKWAVLGTLLQEYGLLQRRLENVEN |
| 557 | IRX4_ | MDSGTRRKNATRETTSTLKAWLQEHRKNPYPTKGEKIMLAIITKMTLTQVS |
| | HUMAN | TWFANARRRLKKENKMTWPPRNKCADEKR |
| 558 | ZBED6_ | NIEKQIYLPSTRAKTSIVWHFFHVDPQYTWRAICNLCEKSVSRGKPGSHLGT |
| | HUMAN | STLQRHLQARHSPHWTRANKFGVASGEE |
| 559 | LHX4_ | AKQNDDSEAGAKRPRTTITAKQLETLKNAYKNSPKPARHVREQLSSETGLD |
| | HUMAN | MRVVQVWFQNRRAKEKRLKKDAGRHRWGQ |
| 560 | SIN3A_ | DALSYLDQVKLQFGSQPQVYNDFLDIMKEFKSQSIDTPGVISRVSQLFKGHP |
| | HUMAN | DLIMGFNTFLPPGYKIEVQTNDMVNVTT |
| 561 | RBBP7_ | DDHTVCLWDINAGPKEGKIVDAKAIFTGHSAVVEDVAWHLLHESLFGSVA |
| | HUMAN | DDQKLMIWDTRSNTTSKPSHLVDAHTAEVN |
| 562 | NKX61_ | GSILLDKDGKRKHTRPTFSGQQIFALEKTFEQTKYLAGPERARLAYSLGMTE |
| | HUMAN | SQVKVWFQNRRTKWRKKHAAEMATAKKK |
| 563 | TRI68_ | DPTALVEAIVEEVACPICMTFLREPMSIDCGHSFCHSCLSGLWEIPGESQNW |
| | HUMAN | GYTCPLCRAPVQPRNLRPNWQLANVVEK |
| 564 | R51A1_ | QSLPKKVSLSSDTTRKPLEIRSPSAESKKPKWVPPAASGGSRSSSSPLVVVSV |
| | HUMAN | KSPNQSLRLGLSRLARVKPLHPNATST |
| 565 | MB3L1_ | AKSSQRKQRDCVNQCKSKPGLSTSIPLRMSSYTFKRPVTRITPHPGNEVRYH |
| | HUMAN | QWEESLEKPQQVCWQRRLQGLQAYSSAG |
| 566 | DLX5_ | VRMVNGKPKKVRKPRTIYSSFQLAALQRRFQKTQYLALPERAELAASLGLT |
| | HUMAN | QTQVKIWFQNKRSKIKKIMKNGEMPPEHS |
| 567 | NOTC1_ | LQCNNHACGWDGGDCSLNFNDPWKNCTQSLQCWKYFSDGHCDSQCNSA |
| | HUMAN | GCLFDGFDCQRAEGQCNPLYDQYCKDHFSDGH |
| 568 | TERF2_ | ETWVEEDELFQVQAAPDEDSTTNITKKQKWTVEESEWVKAGVQKYGEGN |
| | HUMAN | WAAISKNYPFVNRTAVMIKDRWRTMKRLGMN |
| 569 | ZN282_ | AEISLWTVVAAIQAVERKVDAQASQLLNLEGRTGTAEKKLADCEKTAVEF |
| | HUMAN | GNHMESKWAVLGTLLQEYGLLQRRLENLEN |
| 570 | RGS12_ | LEKRTLFRLDLVPINRSVGLKAKPTKPVTEVLRPVVARYGLDLSGLLVRLSG |
| | HUMAN | EKEPLDLGAPISSLDGQRVVLEEKDPSR |
| 571 | ZN840_ | PNCLSSSMQLPHGGGRHQELVRFRDVAVVFSPEEWDHLTPEQRNLYKDVM |
| | HUMAN | LDNCKYLASLGNWTYKAHVMSSLKQGKEPW |
| 572 | SPI2B_ | DDYKEGDLRIMPESSESPPTEREPGGVVDGLIGKHVEYTKEDGSKRIGMVIH |
| | HUMAN | QVEAKPSVYFIKFDDDFHIYVYDLVKKS |
| 573 | PAX7_ | SEPDLPLKRKQRRSRTTFTAEQLEELEKAFERTHYPDIYTREELAQRTKLTE |
| | HUMAN | ARVQVWFSNRRARWRKQAGANQLAAFNH |
| 574 | NKX62_ | AGGVLDKDGKKKHSRPTFSGQQIFALEKTFEQTKYLAGPERARLAYSLGMT |
| | HUMAN | ESQVKVWFQNRRTKWRKRHAVEMASAKKK |
| 575 | ASXL2_ | DVMSFSVTVTTIPASQAMNPSSHGQTIPVQAFSEENSIEGTPSKCYCRLKAMI |
| | HUMAN | MCKGCGAFCHDDCIGPSKLCVSCLVVR |
| 576 | FOXO1_ | GGYSSVSSCNGYGRMGLLHQEKLPSDLDGMFIERLDCDMESIIRNDLMDGD |
| | HUMAN | TLDFNFDNVLPNQSFPHSVKTTTHSWVSG |
| 577 | GATA3_ | GGSPTGFGCKSRPKARSSTGRECVNCGATSTPLWRRDGTGHYLCNACGLY |
| | HUMAN | HKMNGQNRPLIKPKRRLSAARRAGTSCANC |
| 578 | GATA1_ | GQNRPLIRPKKRLIVSKRAGTQCTNCQTTTTTLWRRNASGDPVCNACGLYY |
| | HUMAN | KLHQVNRPLTMRKDGIQTRNRKASGKGKK |
| 579 | ZMYM5_ | PVALLRKQNFQPTAQQQLTKPAKITCANCKKPLQKGQTAYQRKGSAHLFC |
| | HUMAN | STTCLSSFSHKRTQNTRSIICKKDASTKKA |
| 580 | ZN783_ | TEITLWTVVAAIQALEKKVDSCLTRLLTLEGRTGTAEKKLADCEKTAVEFG |
| | HUMAN | NQLEGKWAVLGTLLQEYGLLQRRLENVEN |
| 581 | SPI2B_ | KKQRGRPSSQPRRNIVGCRISHGWKEGDEPITQWKGTVLDQVPINPSLYLV |
| | HUMAN | KYDGIDCVYGLELHRDERVLSLKILSDRV |
| 582 | LRP1_ | WTCDLDDDCGDRSDESASCAYPTCFPLTQFTCNNGRCININWRCDNDNDC |
| | HUMAN | GDNSDEAGCSHSCSSTQFKCNSGRCIPEHW |
| 583 | MIXL1_ | PKGAAAPSASQRRKRTSFSAEQLQLLELVFRRTRYPDIHLRERLAALTLLPE |
| | HUMAN | SRIQVWFQNRRAKSRRQSGKSFQPLARP |
| 584 | SGT1_ | KIKYDWYQTESQVVITLMIKNVQKNDVNVEFSEKELSALVKLPSGEDYNLK |
| | MAN | LELLHPIIPEQSTFKVLSTKIEIKLKKPE |
| 585 | LMCD1_ | DPSKEVEYVCELCKGAAPPDSPVVYSDRAGYNKQWHPTCFVCAKCSEPLV |
| | HUMAN | DLIYFWKDGAPWCGRHYCESLRPRCSGCDE |
| 586 | CEBPA_ | GSGAGKAKKSVDKNSNEYRVRRERNNIAVRKSRDKAKQRNVETQQKVLE |
| | HUMAN | LTSDNDRLRKRVEQLSRELDTLRGIFRQLPE |
| 587 | GATA2_ | GPASSFTPKQRSKARSCSEGRECVNCGATATPLWRRDGTGHYLCNACGLY |
| | HUMAN | HKMNGQNRPLIKPKRRLSAARRAGTCCANC |
| 588 | SOX14_ | KPSDHIKRPMNAFMVWSRGQRRKMAQENPKMHNSEISKRLGAEWKLLSE |
| | HUMAN | AEKRPYIDEAKRLRAQHMKEHPDYKYRPRRK |
| 589 | WTIP_ | LYSGFQQTADKCSVCGHLIMEMILQALGKSYHPGCFRCSVCNECLDGVPFT |
| | HUMAN | VDVENNIYCVRDYHTVFAPKCASCARPIL |
| 590 | PRP19_ | HPSQDLVFSASPDATIRIWSVPNASCVQVVRAHESAVTGLSLHATGDYLLSS |
| | HUMAN | SDDQYWAFSDIQTGRVLTKVTDETSGCS |
| 591 | CBX6_ | ELSAVGERVFAAESIIKRRIRKGRIEYLVKWKGWAIKYSTWEPEENILDSRLI |
| | HUMAN | AAFEQKERERELYGPKKRGPKPKTFLL |
| 592 | NKX11_ | RTGSDSKSGKPRRARTAFTYEQLVALENKFKATRYLSVCERLNLALSLSLTE |
| | HUMAN | TQVKIWFQNRRTKWKKQNPGADTSAPTG |
| 593 | RBBP4_ | VWDLSKIGEEQSPEDAEDGPPELLFIHGGHTAKISDFSWNPNEPWVICSVSE |
| | HUMAN | DNIMQVWQMAENIYNDEDPEGSVDPEGQ |
| 594 | DMRT2_ | ERCTPAGGGAEPRKLSRTPKCARCRNHGVVSCLKGHKRFCRWRDCQCANC |
| | HUMAN | LLVVERQRVMAAQVALRRQQATEDKKGLSG |
| 595 | SMCA2_ | SQPGALIPGDPQAMSQPNRGPSPFSPVQLHQLRAQILAYKMLARGQPLPETL |
| | HUMAN | QLAVQGKRTLPGLQQQQQQQQQQQQQQQ |
| 596 | ZNF10 | MDAKSLTAWSRTLVTFKDVFVDFTREEWKLLDTAQQIVYRNVMLENYKN |
| | | LVSLGYQLTKPDVILRLEKGEEPWLVEREIHQETHPDSETAFEIKSSVSSRSIF |
| | | KDKQSCDIKMEGMARNDLWYLSLEEVWKCRDQLDKYQENPERHLRQVAF |
| | | TQKKVLTQERVSESGKYGGNCLLPAQLVLREYFHKRDSHTKSLKHDLVLN |
| | | GHQDSCASNSNECGQTFCQNIHLIQFARTHTGDKSYKCPDNDNSLTHGSSL |
| | | GISKGIHREKPYECKECGKFFSWRSNLTRHQLIHTGEKPYECKECGKSFSRSS |
| | | HLIGHQKTHTGEEPYECKECGKSFSWFSHLVTHQRTHTGDKLYTCNQCGKS |
| | | FVHSSRLIRHQRTHTGEKPYECPECGKSFRQSTHLILHQRTHVRVRPYECNE |
| | | CGKSYSQRSHLVVHHRIHTGLKPFECKDCGKCFSRSSHLYSHQRTHTGEKP |
| | | YECHDCGKSFSQSSALIVHQRIHTGEKPYECCQCGKAFIRKNDLIKHQRIHV |
| | | GEETYKCNQCGIIFSQNSPFIVHQIAHTGEQFLTCNQCGTALVNTSNLIGYQT |
| | | NHIRENAY |
| 597 | KAP1 | MAASAAAASAAAASAASGSPGPGEGSAGGEKRSTAPSAAASASASAAASSP |
| | | AGGGAEALELLEHCGVCRERLRPEREPRLLPCLHSACSACLGPAAPAAANS |
| | | SGDGGAAGDGTVVDCPVCKQQCFSKDIVENYFMRDSGSKAATDAQDANQ |
| | | CCTSCEDNAPATSYCVECSEPLCETCVEAHQRVKYTKDHTVRSTGPAKSRD |
| | | GERTVYCNVHKHEPLVLFCESCDTLTCRDCQLNAHKDHQYQFLEDAVRNQ |
| | | RKLLASLVKRLGDKHATLQKSTKEVRSSIRQVSDVQKRVQVDVKMAILQI |
| | | MKELNKRGRVLVNDAQKVTEGQQERLERQHWTMTKIQKHQEHILRFASW |
| | | ALESDNNTALLLSKKLIYFQLHRALKMIVDPVEPHGEMKFQWDLNAWTKS |
| | | AEAFGKIVAERPGTNSTGPAPMAPPRAPGPLSKQGSGSSQPMEVQEGYGFG |
| | | SGDDPYSSAEPHVSGVKRSRSGEGEVSGLMRKVPRVSLERLDLDLTADSQP |
| | | PVFKVFPGSTTEDYNLIVIERGAAAAATGQPGTAPAGTPGAPPLAGMAIVKE |
| | | EETEAAIGAPPTATEGPETKPVLMALAEGPGAEGPRLASPSGSTSSGLEVVA |
| | | PEGTSAPGGGPGTLDDSATICRVCQKPGDLVMCNQCEFCFHLDCHLPALQD |
| | | VPGEEWSCSLCHVLPDLKEEDGSLSLDGADSTGVVAKLSPANQRKCERVLL |
| | | ALFCHEPCRPLHQLATDSTFSLDQPGGTLDLTLIRARLQEKLSPPYSSPQEFA |
| | | QDVGRMFKQFNKLTEDKADVQSIIGLQRFFETRMNEAFGDTKFSAVLVEPP |
| | | PMSLPGAGLSSQELSGGPGDGP |
| 598 | MECP2 | MVAGMLGLREEKSEDQDLQGLKDKPLKFKKVKKDKKEEKEGKHEPVQPS |
| | | AHHSAEPAEAGKAETSEGSGSAPAVPEASASPKQRRSIIRDRGPMYDDPTLP |
| | | EGWTRKLKQRKSGRSAGKYDVYLINPQGKAFRSKVELIAYFEKVGDTSLDP |
| | | NDFDFTVTGRGSPSRREQKPPKKPKSPKAPGTGRGRGRPKGSGTTRPKAAT |
| | | SEGVQVKRVLEKSPGKLLVKMPFQTSPGGKAEGGGATTSTQVMVIKRPGR |
| | | KRKAEADPQAIPKKRGRKPGSVVAAAAAEAKKKAVKESSIRSVQETVLPIK |
| | | KRKTRETVSIEVKEVVKPLLVSTLGEKSGKGLKTCKSPGRKSKESSPKGRSS |
| | | SASSPPKKEHHHHHHHSESPKAPVPLLPPLPPPPPEPESSEDPTSPPEPQDLSS |
| | | SVCKEEKMPRGGSLESDGCPKEPAKTQPAVATAATAAEKYKHRGEGERKD |
| | | IVSSSMPRPNREEPVDSRTPVTERVS |
| 599 | human | MSRSRHARPSRLVRKEDVNKKKKNSQLRKTTKGANKNVASVKTLSPGKLK |
| | TET1 | QLIQERDVKKKTEPKPPVPVRSLLTRAGAARMNLDRTEVLFQNPESLTCNG |
| | | FTMALRSTSLSRRLSQPPLVVAKSKKVPLSKGLEKQHDCDYKILPALGVKH |
| | | SENDSVPMQDTQVLPDIETLIGVQNPSLLKGKSQETTQFWSQRVEDSKINIPT |
| | | HSGPAAEILPGPLEGTRCGEGLFSEETLNDTSGSPKMFAQDTVCAPFPQRAT |
| | | PKVTSQGNPSIQLEELGSRVESLKLSDSYLDPIKSEHDCYPTSSLNKVIPDLN |
| | | LRNCLALGGSTSPTSVIKFLLAGSKQATLGAKPDHQEAFEATANQQEVSDT |
| | | TSFLGQAFGAIPHQWELPGADPVHGEALGETPDLPEIPGAIPVQGEVFGTILD |
| | | QQETLGMSGSVVPDLPVFLPVPPNPIATFNAPSKWPEPQSTVSYGLAVQGAI |
| | | QILPLGSGHTPQSSSNSEKNSLPPVMAISNVENEKQVHISFLPANTQGFPLAP |
| | | ERGLFHASLGIAQLSQAGPSKSDRGSSQVSVTSTVHVVNTTVVTMPVPMVS |
| | | TSSSSYTTLLPTLEKKKRKRCGVCEPCQQKTNCGECTYCKNRKNSHQICKK |
| | | RKCEELKKKPSVVVPLEVIKENKRPQREKKPKVLKADFDNKPVNGPKSESM |
| | | DYSRCGHGEEQKLELNPHTVENVTKNEDSMTGIEVEKWTQNKKSQLTDHV |
| | | KGDFSANVPEAEKSKNSEVDKKRTKSPKLFVQTVRNGIKHVHCLPAETNVS |
| | | FKKFNIEEFGKTLENNSYKFLKDTANHKNAMSSVATDMSCDHLKGRSNVL |
| | | VFQQPGFNCSSIPHSSHSIINHHASIHNEGDQPKTPENIPSKEPKDGSPVQPSL |
| | | LSLMKDRRLTLEQVVAIEALTQLSEAPSENSSPSKSEKDEESEQRTASLLNSC |
| | | KAILYTVRKDLQDPNLQGEPPKLNHCPSLEKQSSCNTVVFNGQTTTLSNSHI |
| | | NSATNQASTKSHEYSKVTNSLSLFIPKSNSSKIDTNKSIAQGIITLDNCSNDLH |
| | | QLPPRNNEVEYCNQLLDSSKKLDSDDLSCQDATHTQIEEDVATQLTQLASII |
| | | KINYIKPEDKKVESTPTSLVTCNVQQKYNQEKGTIQQKPPSSVHNNHGSSLT |
| | | KQKNPTQKKTKSTPSRDRRKKKPTVVSYQENDRQKWEKLSYMYGTICDIW |
| | | IASKFQNFGQFCPHDFPTVFGKISSSTKIWKPLAQTRSIMQPKTVFPPLTQIKL |
| | | QRYPESAEEKVKVEPLDSLSLFHLKTESNGKAFTDKAYNSQVQLTVNANQ |
| | | KAHPLTQPSSPPNQCANVMAGDDQIRFQQVVKEQLMHQRLPTLPGISHETP |
| | | LPESALTLRNVNVVCSGGITVVSTKSEEEVCSSSFGTSEFSTVDSAQKNFND |
| | | YAMNFFTNPTKNLVSITKDSELPTCSCLDRVIQKDKGPYYTHLGAGPSVAA |
| | | VREIMENRYGQKGNAIRIEIVVYTGKEGKSSHGCPIAKWVLRRSSDEEKVLC |
| | | LVRQRTGHHCPTAVMVVLIMVWDGIPLPMADRLYTELTENLKSYNGHPTD |
| | | RRCTLNENRTCTCQGIDPETCGASFSFGCSWSMYFNGCKFGRSPSPRRFRID |
| | | PSSPLHEKNLEDNLQSLATRLAPIYKQYAPVAYQNQVEYENVARECRLGSK |
| | | EGRPFSGVTACLDFCAHPHRDIHNMNNGSTVVCTLTREDNRSLGVIPQDEQ |
| | | LHVLPLYKLSDTDEFGSKEGMEAKIKSGAIEVLAPRRKKRTCFTQPVPRSGK |
| | | KRAAMMTEVLAHKIRAVEKKPIPRIKRKNNSTTTNNSKPSSLPTLGSNTETV |
| | | QPEVKSETEPHFILKSSDNTKTYSLMPSAPHPVKEASPGFSWSPKTASATPAP |
| | | LKNDATASCGFSERSSTPHCTMPSGRLSGANAAAADGPGISQLGEVAPLPTL |
| | | SAPVMEPLINSEPSTGVTEPLTPHQPNHQPSFLTSPQDLASSPMEEDEQHSEA |
| | | DEPPSDEPLSDDPLSPAEEKLPHIDEYWSDSEHIFLDANIGGVAIAPAHGSVLI |
| | | ECARRELHATTPVEHPNRNHPTRLSLVFYQHKNLNKPQHGFELNKIKFEAK |
| | | EAKNKKMKASEQKDQAANEGPEQSSEVNELNQIPSHKALTLTHDNVVTVS |
| | | PYALTHVAGPYNHWV |
| 600 | human | MEQDRTNHVEGNRLSPFLIPSPPICQTEPLATKLQNGSPLPERAHPEVNGDT |
| | TET2 | KWHSFKSYYGIPCMKGSQNSRVSPDFTQESRGYSKCLQNGGIKRTVSEPSLS |
| | | GLLQIKKLKQDQKANGERRNFGVSQERNPGESSQPNVSDLSDKKESVSSVA |
| | | QENAVKDFTSFSTHNCSGPENPELQILNEQEGKSANYHDKNIVLLKNKAVL |
| | | MPNGATVSASSVEHTHGELLEKTLSQYYPDCVSIAVQKTTSHINAINSQATN |
| | | ELSCEITHPSHTSGQINSAQTSNSELPPKPAAVVSEACDADDADNASKLAAM |
| | | LNTCSFQKPEQLQQQKSVFEICPSPAENNIQGTTKLASGEEFCSGSSSNLQAP |
| | | GGSSERYLKQNEMNGAYFKQSSVFTKDSFSATTTPPPPSQLLLSPPPPLPQVP |
| | | QLPSEGKSTLNGGVLEEHHHYPNQSNTTLLREVKIEGKPEAPPSQSPNPSTH |
| | | VCSPSPMLSERPQNNCVNRNDIQTAGTMTVPLCSEKTRPMSEHLKHNPPIFG |
| | | SSGELQDNCQQLMRNKEQEILKGRDKEQTRDLVPPTQHYLKPGWIELKAPR |
| | | FHQAESHLKRNEASLPSILQYQPNLSNQMTSKQYTGNSNMPGGLPRQAYTQ |
| | | KTTQLEHKSQMYQVEMNQGQSQGTVDQHLQFQKPSHQVHFSKTDHLPKA |
| | | HVQSLCGTRFHFQQRADSQTEKLMSPVLKQHLNQQASETEPFSNSHLLQHK |
| | | PHKQAAQTQPSQSSHLPQNQQQQQKLQIKNKEEILQTFPHPQSNNDQQREG |
| | | SFFGQTKVEECFHGENQYSKSSEFETHNVQMGLEEVQNINRRNSPYSQTMK |
| | | SSACKIQVSCSNNTHLVSENKEQTTHPELFAGNKTQNLHHMQYFPNNVIPK |
| | | QDLLHRCFQEQEQKSQQASVLQGYKNRNQDMSGQQAAQLAQQRYLIHNH |
| | | ANVFPVPDQGGSHTQTPPQKDTQKHAALRWHLLQKQEQQQTQQPQTESCH |
| | | SQMHRPIKVEPGCKPHACMHTAPPENKTWKKVTKQENPPASCDNVQQKSII |
| | | ETMEQHLKQFHAKSLFDHKALTLKSQKQVKVEMSGPVTVLTRQTTAAELD |
| | | SHTPALEQQTTSSEKTPTKRTAASVLNNFIESPSKLLDTPIKNLLDTPVKTQY |
| | | DFPSCRCVEQIIEKDEGPFYTHLGAGPNVAAIREIMEERFGQKGKAIRIERVI |
| | | YTGKEGKSSQGCPIAKWVVRRSSSEEKLLCLVRERAGHTCEAAVIVILILVW |
| | | EGIPLSLADKLYSELTETLRKYGTLTNRRCALNEERTCACQGLDPETCGASF |
| | | SFGCSWSMYYNGCKFARSKIPRKFKLLGDDPKEEEKLESHLQNLSTLMAPT |
| | | YKKLAPDAYNNQIEYEHRAPECRLGLKEGRPFSGVTACLDFCAHAHRDLH |
| | | NMQNGSTLVCTLTREDNREFGGKPEDEQLHVLPLYKVSDVDEFGSVEAQE |
| | | EKKRSGAIQVLSSFRRKVRMLAEPVKTCRQRKLEAKKAAAEKLSSLENSSN |
| | | KNEKEKSAPSRTKQTENASQAKQLAELLRLSGPVMQQSQQPQPLQKQPPQP |
| | | QQQQRPQQQQPHHPQTESVNSYSASGSTNPYMRRPNPVSPYPNSSHTSDIY |
| | | GSTSPMNFYSTSSQAAGSYLNSSNPMNPYPGLLNQNTQYPSYQCNGNLSVD |
| | | NCSPYLGSYSPQSQPMDLYRYPSQDPLSKLSLPPIHTLYQPRFGNSQSFTSKY |
| | | LGYGNQNMQGDGFSSCTIRPNVHHVGKLPPYPTHEMDGHFMGATSRLPPN |
| | | LSNPNMDYKNGEHHSPSHIIHNYSAAPGMFNSSLHALHLQNKENDMLSHT |
| | | ANGLSKMLPALNHDRTACVQGGLHKLSDANGQEKQPLALVQGVASGAED |
| | | NDEVWSDSEQSFLDPDIGGVAVAPTHGSILIECAKRELHATTPLKNPNRNHP |
| | | TRISLVFYQHKSMNEPKHGLALWEAKMAEKAREKEEECEKYGPDYVPQKS |
| | | HGKKVKREPAEPHETSEPTYLRFIKSLAERTMSVTTDSTVTTSPYAFTRVTG |
| | | PYNRYI |
| 601 | human | MSQFQVPLAVQPDLPGLYDFPQRQVMVGSFPGSGLSMAGSESQLRGGGDG |
| | TET3 | RKKRKRCGTCEPCRRLENCGACTSCTNRRTHQICKLRKCEVLKKKVGLLKE |
| | | VEIKAGEGAGPWGQGAAVKTGSELSPVDGPVPGQMDSGPVYHGDSRQLSA |
| | | SGVPVNGAREPAGPSLLGTGGPWRVDQKPDWEAAPGPAHTARLEDAHDL |
| | | VAFSAVAEAVSSYGALSTRLYETFNREMSREAGNNSRGPRPGPEGCSAGSE |
| | | DLDTLQTALALARHGMKPPNCNCDGPECPDYLEWLEGKIKSVVMEGGEER |
| | | PRLPGPLPPGEAGLPAPSTRPLLSSEVPQISPQEGLPLSQSALSIAKEKNISLQT |
| | | AIAIEALTQLSSALPQPSHSTPQASCPLPEALSPPAPFRSPQSYLRAPSWPVVP |
| | | PEEHSSFAPDSSAFPPATPRTEFPEAWGTDTPPATPRSSWPMPRPSPDPMAEL |
| | | EQLLGSASDYIQSVFKRPEALPTKPKVKVEAPSSSPAPAPSPVLQREAPTPSS |
| | | EPDTHQKAQTALQQHLHHKRSLFLEQVHDTSFPAPSEPSAPGWWPPPSSPV |
| | | PRLPDRPPKEKKKKLPTPAGGPVGTEKAAPGIKPSVRKPIQIKKSRPREAQPL |
| | | FPPVRQIVLEGLRSPASQEVQAHPPAPLPASQGSAVPLPPEPSLALFAPSPSRD |
| | | SLLPPTQEMRSPSPMTALQPGSTGPLPPADDKLEELIRQFEAEFGDSFGLPGP |
| | | PSVPIQDPENQQTCLPAPESPFATRSPKQIKIESSGAVTVLSTTCFHSEEGGQE |
| | | ATPTKAENPLTPTLSGFLESPLKYLDTPTKSLLDTPAKRAQAEFPTCDCVEQI |
| | | VEKDEGPYYTHLGSGPTVASIRELMEERYGEKGKAIRIEKVIYTGKEGKSSR |
| | | GCPIAKWVIRRHTLEEKLLCLVRHRAGHHCQNAVIVILILAWEGIPRSLGDT |
| | | LYQELTDTLRKYGNPTSRRCGLNDDRTCACQGKDPNTCGASFSFGCSWSM |
| | | YFNGCKYARSKTPRKFRLAGDNPKEEEVLRKSFQDLATEVAPLYKRLAPQA |
| | | YQNQVTNEEIAIDCRLGLKEGRPFAGVTACMDFCAHAHKDQHNLYNGCTV |
| | | VCTLTKEDNRCVGKIPEDEQLHVLPLYKMANTDEFGSEENQNAKVGSGAIQ |
| | | VLTAFPREVRRLPEPAKSCRQRQLEARKAAAEKKKIQKEKLSTPEKIKQEAL |
| | | ELAGITSDPGLSLKGGLSQQGLKPSLKVEPQNHFSSFKYSGNAVVESYSVLG |
| | | NCRPSDPYSMNSVYSYHSYYAQPSLTSVNGFHSKYALPSFSYYGFPSSNPVF |
| | | PSQFLGPGAWGHSGSSGSFEKKPDLHALHNSLSPAYGGAEFAELPSQAVPT |
| | | DAHHPTPHHQQPAYPGPKEYLLPKAPLLHSVSRDPSPFAQSSNCYNRSIKQE |
| | | PVDPLTQAEPVPRDAGKMGKTPLSEVSQNGGPSHLWGQYSGGPSMSPKRT |
| | | NGVGGSWGVFSSGESPAIVPDKLSSFGASCLAPSHFTDGQWGLFPGEGQQA |
| | | ASHSGGRLRGKPWSPCKFGNSTSALAGPSLTEKPWALGAGDFNSALKGSPG |
| | | FQDKLWNPMKGEEGRIPAAGASQLDRAWQSFGLPLGSSEKLFGALKSEEKL |
| | | WDPFSLEEGPAEEPPSKGAVKEEKGGGGAEEEEEELWSDSEHNFLDENIGG |
| | | VAVAPAHGSILIECARRELHATTPLKKPNRCHPTRISLVFYQHKNLNQPNHG |
| | | LALWEAKMKQLAERARARQEEAARLGLGQQEAKLYGKKRKWGGTVVAE |
| | | PQQKEKKGVVPTRQALAVPTDSAVTVSSYAYTKVTGPYSRWI |
| 502 | human | MEAENAGSYSLQQAQAFYTFPFQQLMAEAPNMAVVNEQQMPEEVPAPAP |
| | TDG | AQEPVQEAPKGRKRKPRTTEPKQPVEPKKPVESKKSGKSAKSKEKQEKITD |
| | | TFKVKRKVDRFNGVSEAELLTKTLPDILTFNLDIVIIGINPGLMAAYKGHHY |
| | | PGPGNHFWKCLFMSGLSEVQLNHMDDHTLPGKYGIGFTNMVERTTPGSKD |
| | | LSSKEFREGGRILVQKLQKYQPRIAVFNGKCIYEIFSKEVFGVKVKNLEFGL |
| | | QPHKIPDTETLCYVMPSSSARCAQFPRAQDKVHYYIKLKDLRDQLKGIERN |
| | | MDVQEVQYTFDLQLAQEDAKKMAVKEEKYDPGYEAAYGGAYGENPCSSE |
| | | PCGFSSNGLIESVELRGESAFSGIPNGQWMTQSFTDQIPSFSNHCGTQEQEEE |
| | | SHA |
| 603 | arabidopsis | MEKQRREESSFQQPPWIPQTPMKPFSPICPYTVEDQYHSSQLEERRFVGNKD |
| | ROS1 | MSGLDHLSFGDLLALANTASLIFSGQTPIPTRNTEVMQKGTEEVESLSSVSN |
| | | NVAEQILKTPEKPKRKKHRPKVRREAKPKREPKPRAPRKSVVTDGQESKTP |
| | | KRKYVRKKVEVSKDQDATPVESSAAVETSTRPKRLCRRVLDFEAENGENQ |
| | | TNGDIREAGEMESALQEKQLDSGNQELKDCLLSAPSTPKRKRSQGKRKGV |
| | | QPKKNGSNLEEVDISMAQAAKRRQGPTCCDMNLSGIQYDEQCDYQKMHW |
| | | LYSPNLQQGGMRYDAICSKVFSGQQHNYVSAFHATCYSSTSQLSANRVLTV |
| | | EERREGIFQGRQESELNVLSDKIDTPIKKKTTGHARFRNLSSMNKLVEVPEH |
| | | LTSGYCSKPQQNNKILVDTRVTVSKKKPTKSEKSQTKQKNLLPNLCRFPPSF |
| | | TGLSPDELWKRRNSIETISELLRLLDINREHSETALVPYTMNSQIVLFGGGAG |
| | | AIVPVTPVKKPRPRPKVDLDDETDRVWKLLLENINSEGVDGSDEQKAKWW |
| | | EEERNVFRGRADSFIARMHLVQGDRRFTPWKGSVVDSVVGVFLTQNVSDH |
| | | LSSSAFMSLASQFPVPFVPSSNFDAGTSSMPSIQITYLDSEETMSSPPDHNHSS |
| | | VTLKNTQPDEEKDYVPSNETSRSSSEIAISAHESVDKTTDSKEYVDSDRKGS |
| | | SVEVDKTDEKCRVLNLFPSEDSALTCQHSMVSDAPQNTERAGSSSEIDLEGE |
| | | YRTSFMKLLQGVQVSLEDSNQVSPNMSPGDCSSEIKGFQSMKEPTKSSVDS |
| | | SEPGCCSQQDGDVLSCQKPTLKEKGKKVLKEEKKAFDWDCLRREAQARA |
| | | GIREKTRSTMDTVDWKAIRAADVKEVAETIKSRGMNHKLAERIQGFLDRLV |
| | | NDHGSIDLEWLRDVPPDKAKEYLLSFNGLGLKSVECVRLLTLHHLAFPVDT |
| | | NVGRIAVRLGWVPLQPLPESLQLHLLEMYPMLESIQKYLWPRLCKLDQKTL |
| | | YELHYQMITFGKVFCTKSKPNCNACPMKGECRHFASAFASARLALPSTEKG |
| | | MGTPDKNPLPLHLPEPFQREQGSEVVQHSEPAKKVTCCEPIIEEPASPEPETA |
| | | EVSIADIEEAFFEDPEEIPTIRLNMDAFTSNLKKIMEHNKELQDGNMSSALVA |
| | | LTAETASLPMPKLKNISQLRTEHRVYELPDEHPLLAQLEKREPDDPCSYLLA |
| | | IWTPGETADSIQPSVSTCIFQANGMLCDEETCFSCNSIKETRSQIVRGTILIPCR |
| | | TAMRGSFPLNGTYFQVNEVFADHASSLNPINVPRELIWELPRRTVYFGTSVP |
| | | TIFKGLSTEKIQACFWKGYVCVRGFDRKTRGPKPLIARLHFPASKLKGQQA |
| | | NLA |
| 604 | arabidopsis | MNSRADPGDRYFRVPLENQTQQEFMGSWIPFTPKKPRSSLMVDERVINQDL |
| | DME | NGFPGGEFVDRGFCNTGVDHNGVFDHGAHQGVTNLSMMINSLAGSHAQA |
| | | WSNSERDLLGRSEVTSPLAPVIRNTTGNVEPVNGNFTSDVGMVNGPFTQSG |
| | | TSQAGYNEFELDDLLNPDQMPFSFTSLLSGGDSLFKVRQYGPPACNKPLYN |
| | | LNSPIRREAVGSVCESSFQYVPSTPSLFRTGEKTGFLEQIVTTTGHEIPEPKSD |
| | | KSMQSIMDSSAVNATEATEQNDGSRQDVLEFDLNKTPQQKPSKRKRKFMP |
| | | KVVVEGKPKRKPRKPAELPKVVVEGKPKRKPRKAATQEKVKSKETGSAKK |
| | | KNLKESATKKPANVGDMSNKSPEVTLKSCRKALNFDLENPGDARQGDSES |
| | | EIVQNSSGANSFSEIRDAIGGTNGSFLDSVSQIDKTNGLGAMNQPLEVSMGN |
| | | QPDKLSTGAKLARDQQPDLLTRNQQCQFPVATQNTQFPMENQQAWLQMK |
| | | NQLIGFPFGNQQPRMTIRNQQPCLAMGNQQPMYLIGTPRPALVSGNQQLGG |
| | | PQGNKRPIFLNHQTCLPAGNQLYGSPTDMHQLVMSTGGQQHGLLIKNQQP |
| | | GSLIRGQQPCVPLIDQQPATPKGFTHLNQMVATSMSSPGLRPHSQSQVPTTY |
| | | LHVESVSRILNGTTGTCQRSRAPAYDSLQQDIHQGNKYILSHEISNGNGCKK |
| | | ALPQNSSLPTPIMAKLEEARGSKRQYHRAMGQTEKHDLNLAQQIAQSQDV |
| | | ERHNSSTCVEYLDAAKKTKIQKVVQENLHGMPPEVIEIEDDPTDGARKGKN |
| | | TASISKGASKGNSSPVKKTAEKEKCIVPKTPAKKGRAGRKKSVPPPAHASEI |
| | | QLWQPTPPKTPLSRSKPKGKGRKSIQDSGKARGPSGELLCQDSIAEIIYRMQ |
| | | NLYLGDKEREQEQNAMVLYKGDGALVPYESKKRKPRPKVDIDDETTRIWN |
| | | LLMGKGDEKEGDEEKDKKKEKWWEEERRVFRGRADSFIARMHLVQGDRR |
| | | FSPWKGSVVDSVIGVFLTQNVSDHLSSSAFMSLAARFPPKLSSSREDERNVR |
| | | SVVVEDPEGCILNLNEIPSWQEKVQHPSDMEVSGVDSGSKEQLRDCSNSGIE |
| | | RFNFLEKSIQNLEEEVLSSQDSFDPAIFQSCGRVGSCSCSKSDAEFPTTRCET |
| | | KTVSGTSQSVQTGSPNLSDEICLQGNERPHLYEGSGDVQKQETTNVAQKKP |
| | | DLEKTMNWKDSVCFGQPRNDTNWQTTPSSSYEQCATRQPHVLDIEDFGMQ |
| | | GEGLGYSWMSISPRVDRVKNKNVPRRFFRQGGSVPREFTGQIIPSTPHELPG |
| | | MGLSGSSSAVQEHQDDTQHNQQDEMNKASHLQKTFLDLLNSSEECLTRQS |
| | | STKQNITDGCLPRDRTAEDVVDPLSNNSSLQNILVESNSSNKEQTAVEYKET |
| | | NATILREMKGTLADGKKPTSQWDSLRKDVEGNEGRQERNKNNMDSIDYEA |
| | | IRRASISEISEAIKERGMNNMLAVRIKDFLERIVKDHGGIDLEWLRESPPDKA |
| | | KDYLLSIRGLGLKSVECVRLLTLHNLAFPVDTNVGRIAVRMGWVPLQPLPE |
| | | SLQLHLLELYPVLESIQKFLWPRLCKLDQRTLYELHYQLITFGKVFCTKSRP |
| | | NCNACPMRGECRHFASAYASARLALPAPEERSLTSATIPVPPESYPPVAIPMI |
| | | ELPLPLEKSLASGAPSNRENCEPIIEEPASPGQECTEITESDIEDAYYNEDPDEI |
| | | PTIKLNIEQFGMTLREHMERNMELQEGDMSKALVALHPTTTSIPTPKLKNIS |
| | | RLRTEHQVYELPDSHRLLDGMDKREPDDPSPYLLAIWTPGETANSAQPPEQ |
| | | KCGGKASGKMCFDETCSECNSLREANSQTVRGTLLIPCRTAMRGSFPLNGT |
| | | YFQVNELFADHESSLKPIDVPRDWIWDLPRRTVYFGTSVTSIFRGLSTEQIQF |
| | | CFWKGFVCVRGFEQKTRAPRPLMARLHFPASKLKNNKT |
| 605 | arabidopsis | MEVEGEVREKEARVKGRQPETEVLHGLPQEQSIFNNMQHNHQPDSDRRRL |
| | DML2 | SLENLPGLYNMSCTQLLALANATVATGSSIGASSSSLSSQHPTDSWINSWK |
| | | MDSNPWTLSKMQKQQYDVSTPQKFLCDLNLTPEELVSTSTQRTEPESPQITL |
| | | KTPGKSLSETDHEPHDRIKKSVLGTGSPAAVKKRKIARNDEKSQLETPTLKR |
| | | KKIRPKVVREGKTKKASSKAGIKKSSIAATATKTSEESNYVRPKRLTRRSIRF |
| | | DFDLQEEDEEFCGIDFTSAGHVEGSSGEENLTDTTLGMFGHVPKGRRGQRR |
| | | SNGFKKTDNDCLSSMLSLVNTGPGSFMESEEDRPSDSQISLGRQRSIMATRP |
| | | RNFRSLKKLLQRIIPSKRDRKGCKLPRGLPKLTVASKLQLKVFRKKRSQRNR |
| | | VASQFNARILDLQWRRQNPTGTSLADIWERSLTIDAITKLFEELDINKEGLCL |
| | | PHNRETALILYKKSYEEQKAIVKYSKKQKPKVQLDPETSRVWKLLMSSIDC |
| | | DGVDGSDEEKRKWWEEERNMFHGRANSFIARMRVVQGNRTFSPWKGSVV |
| | | DSVVGVFLTQNVADHSSSSAYMDLAAEFPVEWNFNKGSCHEEWGSSVTQE |
| | | TILNLDPRTGVSTPRIRNPTRVIIEEIDDDENDIDAVCSQESSKTSDSSITSADQ |
| | | SKTMLLDPFNTVLMNEQVDSQMVKGKGHIPYTDDLNDLSQGISMVSSAST |
| | | HCELNLNEVPPEVELCSHQQDPESTIQTQDQQESTRTEDVKKNRKKPTTSKP |
| | | KKKSKESAKSTQKKSVDWDSLRKEAESGGRKRERTERTMDTVDWDALRC |
| | | TDVHKIANIIIKRGMNNMLAERIKAFLNRLVKKHGSIDLEWLRDVPPDKAK |
| | | EYLLSINGLGLKSVECVRLLSLHQIAFPVDTNVGRIAVRLGWVPLQPLPDEL |
| | | QMHLLELYPVLESVQKYLWPRLCKLDQKTLYELHYHMITFGKVFCTKVKP |
| | | NCNACPMKAECRHYSSARASARLALPEPEESDRTSVMIHERRSKRKPVVVN |
| | | FRPSLFLYQEKEQEAQRSQNCEPIIEEPASPEPEYIEHDIEDYPRDKNNVGTSE |
| | | DPWENKDVIPTIILNKEAGTSHDLVVNKEAGTSHDLVVLSTYAAAIPRRKLK |
| | | IKEKLRTEHHVFELPDHHSILEGFERREAEDIVPYLLAIWTPGETVNSIQPPK |
| | | QRCALFESNNTLCNENKCFQCNKTREEESQTVRGTILIPCRTAMRGGFPLNG |
| | | TYFQTNEVFADHDSSINPIDVPTELIWDLKRRVAYLGSSVSSICKGLSVEAIK |
| | | YNFQEGYVCVRGFDRENRKPKSLVKRLHCSHVAIRTKEKTEE |
| 606 | arabidopsis | MLTDGSQHTYQNGETKNSKEHERKCDESAHLQDNSQTTHKKKEKKNSKE |
| | DML3 | KHGIKHSESEHLQDDISQRVTGKGRRRNSKGTPKKLRFNRPRILEDGKKPRN |
| | | PATTRLRTISNKRRKKDIDSEDEVIPELATPTKESFPKRRKNEKIKRSVARTL |
| | | NFKQEIVLSCLEFDKICGPIFPRGKKRTTTRRRYDFLCFLLPMPVWKKQSRR |
| | | SKRRKNMVRWARIASSSKLLEETLPLIVSHPTINGQADASLHIDDTLVRHVV |
| | | SKQTKKSANNVIEHLNRQITYQKDHGLSSLADVPLHIEDTLIKSASSVLSERP |
| | | IKKTKDIAKLIKDMGRLKINKKVTTMIKADKKLVTAKVNLDPETIKEWDVL |
| | | MVNDSPSRSYDDKETEAKWKKEREIFQTRIDLFINRMHRLQGNRKFKQWK |
| | | GSVVDSVVGVFLTQNTTDYLSSNAFMSVAAKFPVDAREGLSYYIEEPQDAK |
| | | SSECIILSDESISKVEDHENTAKRKNEKTGIIEDEIVDWNNLRRMYTKEGSRP |
| | | EMHMDSVNWSDVRLSGQNVLETTIKKRGQFRILSERILKFLNDEVNQNGNI |
| | | DLEWLRNAPSHLVKRYLLEIEGIGLKSAECVRLLGLKHHAFPVDTNVGRIA |
| | | VRLGLVPLEPLPNGVQMHQLFEYPSMDSIQKYLWPRLCKLPQETLYELHYQ |
| | | MITFGKVFCTKTIPNCNACPMKSECKYFASAYVSSKVLLESPEEKMHEPNTF |
| | | MNAHSQDVAVDMTSNINLVEECVSSGCSDQAICYKPLVEFPSSPRAEIPEST |
| | | DIEDVPFMNLYQSYASVPKIDFDLDALKKSVEDALVISGRMSSSDEEISKAL |
| | | VIPTPENACIPIKPPRKMKYYNRLRTEHVVYVLPDNHELLHDFERRKLDDPS |
| | | PYLLAIWQPGETSSSFVPPKKKCSSDGSKLCKIKNCSYCWTIREQNSNIFRGT |
| | | ILIPCRTAMRGAFPLNGTYFQTNEVFADHETSLNPIVFRRELCKGLEKRALY |
| | | CGSTVTSIFKLLDTRRIELCFWTGFLCLRAFDRKQRDPKELVRRLHTPPDER |
| | | GPKFMSDDDI |
| 607 | Herpes | MDLLVDELFADMNADGASPPPPRPAGGPKNTPAAPPLYATGRLSQAQLMP |
| | strain 17 | SPPMPVPPAALFNRLLDDLGFSAGPALCTMLDTWNEDLFSALPTNADLYRE |
| | VP16 | CKFLSTLPSDVVEWGDAYVPERTQIDIRAHGDVAFPTLPATRDGLGLYYEA |
| | | LSRFFHAELRAREESYRTVLANFCSALYRYLRASVRQLHRQAHMRGRDRD |
| | | LGEMLRATIADRYYRETARLARVLFLHLYLFLTREILWAAYAEQMMRPDL |
| | | FDCLCCDLESWRQLAGLFQPFMFVNGALTVRGVPIEARRLRELNHIREHLN |
| | | LPLVRSAATEEPGAPLTTPPTLHGNQARASGYFMVLIRAKLDSYSSFTTSPSE |
| | | AVMREHAYSRARTKNNYGSTIEGLLDLPDDDAPEEAGLAAPRLSFLPAGHT |
| | | RRLSTAPPTDVSLGDELHLDGEDVAMAHADALDDFDLDMLGDGDSPGPGF |
| | | TPHDSAPYGALDMADFEFEQMFTDALGIDEYGG |
| 608 | Herpes | DALDDFDLDMLGSDALDDFDLDMLGSDALDDFDLDMLGSDALDDFDLDM |
| | strain 17 | |
| | VP64 | |
| 609 | Herpes | DALDDFDLDMLGSDALDDFDLDMLGSDALDDFDLDMLGSDALDDFDLDM |
| | strain 17 | LGSDALDDFDLDMLGSSDALDDFDLDMLGSDALDDFDLDMLGSDALDDF |
| | VP160 | DLDMLGSDALDDFDLDMLGSDALDDFDLDML |
| 610 | human | MEGAGGANDKKKISSERRKEKSRDAARSRRSKESEVFYELAHQLPLPHNVS |
| | HIF1alpha | SHLDKASVMRLTISYLRVRKLLDAGDLDIEDDMKAQMNCFYLKALDGFV |
| | | MVLTDDGDMIYISDNVNKYMGLTQFELTGHSVFDFTHPCDHEEMREMLTH |
| | | RNGLVKKGKEQNTQRSFFLRMKCTLTSRGRTMNIKSATWKVLHCTGHIHV |
| | | YDTNSNQPQCGYKKPPMTCLVLICEPIPHPSNIEIPLDSKTFLSRHSLDMKFS |
| | | YCDERITELMGYEPEELLGRSIYEYYHALDSDHLTKTHHDMFTKGQVTTGQ |
| | | YRMLAKRGGYVWVETQATVIYNTKNSQPQCIVCVNYVVSGIIQHDLIFSLQ |
| | | QTECVLKPVESSDMKMTQLFTKVESEDTSSLFDKLKKEPDALTLLAPAAGD |
| | | TIISLDFGSNDTETDDQQLEEVPLYNDVMLPSPNEKLQNINLAMSPLPTAETP |
| | | KPLRSSADPALNQEVALKLEPNPESLELSFTMPQIQDQTPSPSDGSTRQSSPE |
| | | PNSPSEYCFYVDSDMVNEFKLELVEKLFAEDTEAKNPFSTQDTDLDLEMLA |
| | | PYIPMDDDFQLRSFDQLSPLESSSASPESASPQSTVTVFQQTQIQEPTANATT |
| | | TTATTDELKTVTKDRMEDIKILIASPSPTHIHKETTSATSSPYRDTQSRTASPN |
| | | RAGKGVIEQTEKSHPRSPNVLSVALSQRTTVPEEELNPKILALQNAQRKRK |
| | | MEHDGSLFQAVGIGTLLQQPDDHAATTSLSWKRVKGCKSSEQNGMEQKTII |
| | | LIPSDLACRLLGQSMDESGLPQLTSYDCEVNAPIQGSRNLLQGEELLRALDQ |
| | | VN |
| 611 | human | MADHMMAMNHGRFPDGTNGLHHHPAHRMGMGQFPSPHHHQQQQPQHA |
| | CITED2 | FNALMGEHIHYGAGNMNATSGIRHAMGPGTVNGGHPPSALAPAARFNNSQ |
| | | FMGPPVASQGGSLPASMQLQKLNNQYFNHHPYPHNHYMPDLHPAAGHQM |
| | | NGTNQHFRDCNPKHSGGSSTPGGSGGSSTPGGSGSSSGGGAGSSNSGGGSG |
| | | SGNMPASVAHVPAAMLPPNVIDTDFIDEEVLMSLVIEMGLDRIKELPELWL |
| | | GQNEFDFMTDFVCKQQPSRVSC |
| 612 | human | MAQWNQLQQLDTRYLEQLHQLYSDSFPMELRQFLAPWIESQDWAYAASK |
| | Stat3 | ESHATLVFHNLLGEIDQQYSRFLQESNVLYQHNLRRIKQFLQSRYLEKPMEI |
| | | ARIVARCLWEESRLLQTAATAAQQGGQANHPTAAVVTEKQQMLEQHLQD |
| | | VRKRVQDLEQKMKVVENLQDDFDFNYKTLKSQGDMQDLNGNNQSVTRQ |
| | | KMQQLEQMLTALDQMRRSIVSELAGLLSAMEYVQKTLTDEELADWKRRQ |
| | | QIACIGGPPNICLDRLENWITSLAESQLQTRQQIKKLEELQQKVSYKGDPIVQ |
| | | HRPMLEERIVELFRNLMKSAFVVERQPCMPMHPDRPLVIKTGVQFTTKVRL |
| | | LVKFPELNYQLKIKVCIDKDSGDVAALRGSRKFNILGTNTKVMNMEESNNG |
| | | SLSAEFKHLTLREQRCGNGGRANCDASLIVTEELHLITFETEVYHQGLKIDL |
| | | ETHSLPVVVISNICQMPNAWASILWYNMLTNNPKNVNFFTKPPIGTWDQVA |
| | | EVLSWQFSSTTKRGLSIEQLTTLAEKLLGPGVNYSGCQITWAKFCKENMAG |
| | | KGFSFWVWLDNIIDLVKKYILALWNEGYIMGFISKERERAILSTKPPGTFLLR |
| | | FSESSKEGGVTFTWVEKDISGKTQIQSVEPYTKQQLNNMSFAEIIMGYKIMD |
| | | ATNILVSPLVYLYPDIPKEEAFGKYCRPESQEHPEADPGSAAPYLKTKFICVT |
| | | PTTCSNTIDLPMSPRTLDSLMQFGNNGEGAEPSAGGQFESLTFDMELTSECA |
| | | TSPM |
| 613 | human p65 | MDELFPLIFPAEPAQASGPYVEIIEQPKQRGMRFRYKCEGRSAGSIPGERSTD |
| | | TTKTHPTIKINGYTGPGTVRISLVTKDPPHRPHPHELVGKDCRDGFYEAELC |
| | | PDRCIHSFQNLGIQCVKKRDLEQAISQRIQTNNNPFQVPIEEQRGDYDLNAV |
| | | RLCFQVTVRDPSGRPLRLPPVLSHPIFDNRAPNTAELKICRVNRNSGSCLGG |
| | | DEIFLLCDKVQKEDIEVYFTGPGWEARGSFSQADVHRQVAIVFRTPPYADPS |
| | | LQAPVRVSMQLRRPSDRELSEPMEFQYLPDTDDRHRIEEKRKRTYETFKSIM |
| | | KKSPFSGPTDPRPPPRRIAVPSRSSASVPKPAPQPYPFTSSLSTINYDEFPTMV |
| | | FPSGQISQASALAPAPPQVLPQAPAPAPAPAMVSALAQAPAPVPVLAPGPPQ |
| | | AVAPPAPKPTQAGEGTLSEALLQLQFDDEDLGALLGNSTDPAVFTDLASVD |
| | | NSEFQQLLNQGIPVAPHTTEPMLMEYPEAITRLVTGAQRPPDPAPAPLGAPG |
| | | LPNGLLSGDEDFSSIADMDFSALLSQISS |
| 614 | human p53 | MEEPQSDPSVEPPLSQETFSDLWKLLPENNVLSPLPSQAMDDLMLSPDDIEQ |
| | | WFTEDPGPDEAPRMPEAAPPVAPAPAAPTPAAPAPAPSWPLSSSVPSQKTY |
| | | QGSYGFRLGFLHSGTAKSVTCTYSPALNKMFCQLAKTCPVQLWVDSTPPPG |
| | | TRVRAMAIYKQSQHMTEVVRRCPHHERCSDSDGLAPPQHLIRVEGNLRVE |
| | | YLDDRNTFRHSVVVPYEPPEVGSDCTTIHYNYMCNSSCMGGMNRRPILTIIT |
| | | LEDSSGNLLGRNSFEVRVCACPGRDRRTEEENLRKKGEPHHELPPGSTKRA |
| | | LPNNTSSSPQPKKKPLDGEYFTLQIRGRERFEMFRELNEALELKDAQAGKEP |
| | | GGSRAHSSHLKSKKGQSTSRHKKLMFKTEGPDSD |
| 615 | human | MAEEFVTLKDVGMDFTLGDWEQLGLEQGDTFWDTALDNCQDLFLLDPPR |
| | ZNF473 | PNLTSHPDGSEDLEPLAGGSPEATSPDVTETKNSPLMEDFFEEGFSQEIIEML |
| | | SKDGFWNSNFGEACIEDTWLDSLLGDPESLLRSDIATNGESPTECKSHELKR |
| | | GLSPVSTVSTGEDSMVHNVSEKTLTPAKSKEYRGEFFSYSDHSQQDSVQEG |
| | | EKPYQCSECGKSFSGSYRLTQHWITHTREKPTVHQECEQGFDRNASLSVYP |
| | | KTHTGYKFYVCNEYGTTFSQSTYLWHQKTHTGEKPCKSQDSDHPPSHDTQ |
| | | PGEHQKTHTDSKSYNCNECGKAFTRIFHLTRHQKIHTRKRYECSKCQATFN |
| | | LRKHLIQHQKTHAAKTTSECQECGKIFRHSSLLIEHQALHAGEEPYKCNERG |
| | | KSFRHNSTLKIHQRVHSGEKPYKCSECGKAFHRHTHLNEHRRIHTGYRPHK |
| | | CQECVRSFSRPSHLMRHQAIHTAEKPYSCAECKETFSDNNRLVQHQKMHT |
| | | VKTPYECQECGERFICGSTLKCHESVHAREKQGFFVSGKILDQNPEQKEKCF |
| | | KCNKCEKTFSCSKYLTQHERIHTRGVKPFECDQCGKAFGQSTRLIHHQRIHS |
| | | RVRLYKWGEQGKAISSASLIKLQSFHTKEHPFKCNECGKTFSHSAHLSKHQ |
| | | LIHAGENPFKCSKCDRVFTQRNYLVQHERTHARKKPLVCNECGKTFRQSSC |
| | | LSKHQRIHSGEKPYVCDYCGKAFGLSAELVRHQRIHTGEKPYVCQECGKAF |
| | | TQSSCLSIHRRVHTGEKPYRCGECGKAFAQKANLTQHQRIHTGEKPYSCNV |
| | | CGKAFVLSAHLNQHLRVHTQETLYQCQRCQKAFRCHSSLSRHQRVHNKQQ |
| | | YCL |
| 616 | human | MAEAPQVVEIDPDFEPLPRPRSCTWPLPRPEFSQSNSATSSPAPSGSAAANPD |
| | FOXO1 | AAAGLPSASAAAVSADFMSNLSLLEESEDFPQAPGSVAAAVAAAAAAAAT |
| | | GGLCGDFQGPEAGCLHPAPPQPPPPGPLSQHPPVPPAAAGPLAGQPRKSSSS |
| | | RRNAWGNLSYADLITKAIESSAEKRLTLSQIYEWMVKSVPYFKDKGDSNSS |
| | | AGWKNSIRHNLSLHSKFIRVQNEGTGKSSWWMLNPEGGKSGKSPRRRAAS |
| | | MDNNSKFAKSRSRAAKKKASLQSGQEGAGDSPGSQFSKWPASPGSHSNDD |
| | | FDNWSTFRPRTSSNASTISGRLSPIMTEQDDLGEGDVHSMVYPPSAAKMAS |
| | | TLPSLSEISNPENMENLLDNLNLLSSPTSLTVSTQSSPGTMMQQTPCYSFAPP |
| | | NTSLNSPSPNYQKYTYGQSSMSPLPQMPIQTLQDNKSSYGGMSQYNCAPGL |
| | | LKELLTSDSPPHNDIMTPVDPGVAQPNSRVLGQNVMMGPNSVMSTYGSQA |
| | | SHNKMMNPSSHTHPGHAQQTSAVNGRPLPHTVSTMPHTSGMNRLTQVKTP |
| | | VQVPLPHPMQMSALGGYSSVSSCNGYGRMGLLHQEKLPSDLDGMFIERLD |
| | | CDMESIIRNDLMDGDTLDFNFDNVLPNQSFPHSVKTTTHSWVSG |
| 617 | human | MARRPRHSIYSSDEDDEDFEMCDHDYDGLLPKSGKRHLGKTRWTREEDEK |
| | Myb | LKKLVEQNGTDDWKVIANYLPNRTDVQCQHRWQKVLNPELIKGPWTKEE |
| | | DQRVIELVQKYGPKRWSVIAKHLKGRIGKQCRERWHNHLNPEVKKTSWTE |
| | | EEDRIIYQAHKRLGNRWAEIAKLLPGRTDNAIKNHWNSTMRRKVEQEGYL |
| | | QESSKASQPAVATSFQKNSHLMGFAQAPPTAQLPATGQPTVNNDYSYYHIS |
| | | EAQNVSSHVPYPVALHVNIVNVPQPAAAAIQRHYNDEDPEKEKRIKELELL |
| | | LMSTENELKGQQVLPTQNHTCSYPGWHSTTIADHTRPHGDSAPVSCLGEHH |
| | | STPSLPADPGSLPEESASPARCMIVHQGTILDNVKNLLEFAETLQFIDSFLNTS |
| | | SNHENSDLEMPSLTSTPLIGHKLTVTTPFHRDQTVKTQKENTVFRTPAIKRSI |
| | | LESSPRTPTPFKHALAAQEIKYGPLKMLPQTPSHLVEDLQDVIKQESDESGIV |
| | | AEFQENGPPLLKKIKQEVESPTDKSGNFFCSHHWEGDSLNTQLFTQTSPVAD |
| | | APNILTSSVLMAPASEDEDNVLKAFTVPKNRSLASPLQATKAQRLFQF |
| 618 | human | MATSNNPRKFSEKIALHNQKQAEETAAFEEVMKDLSLTRAARLQLQKSQY |
| | CRTC1 | LQLGPSRGQYYGGSLPNVNQIGSGTMDLPFQTPFQSSGLDTSRTTRHHGLV |
| | | DRVYRERGRLGSPHRRPLSVDKHGRQADSCPYGTMYLSPPADTSWRRTNS |
| | | DSALHQSTMTPTQPESFSSGSQDVHQKRVLLLTVPGMEETTSEADKNLSKQ |
| | | AWDTKKTGSRPKSCEVPGINIFPSADQENTTALIPATHNTGGSLPDLTNIHFP |
| | | SPLPTPLDPEEPTFPALSSSSSTGNLAANLTHLGIGGAGQGMSTPGSSPQHRP |
| | | AGVSPLSLSTEARRQQASPTLSPLSPITQAVAMDALSLEQQLPYAFFTQAGS |
| | | QQPPPQPQPPPPPPPASQQPPPPPPPQAPVRLPPGGPLLPSASLTRGPQPPPLA |
| | | VTVPSSLPQSPPENPGQPSMGIDIASAPALQQYRTSAGSPANQSPTSPVSNQG |
| | | FSPGSSPQHTSTLGSVFGDAYYEQQMAARQANALSHQLEQFNMMENAISSS |
| | | SLYSPGSTLNYSQAAMMGLTGSHGSLPDSQQLGYASHSGIPNIILTVTGESPP |
| | | SLSKELTSSLAGVGDVSFDSDSQFPLDELKIDPLTLDGLHMLNDPDMVLADP |
| | | ATEDTFRMDRL |
| 619 | human | MASAGVAAGRQAEDVLPPTSDQPLPDTKPLPPPQPPPVPAPQPQQSPAPRPQ |
| | Med9 | SPARAREEENYSFLPLVHNIIKCMDKDSPEVHQDLNALKSKFQEMRKLISTM |
| | | PGIHLSPEQQQQQLQSLREQVRTKNELLQKYKSLCMFEIPKE |
| 620 | human | MTGKLAEKLPVTMSSLLNQLPDNLYPEEIPSALNLFSGSSDSVVHYNQMAT |
| | EGR3 | ENVMDIGLTNEKPNPELSYSGSFQPAPGNKTVTYLGKFAFDSPSNWCQDNII |
| | | SLMSAGILGVPPASGALSTQTSTASMVQPPQGDVEAMYPALPPYSNCGDLY |
| | | SEPVSFHDPQGNPGLAYSPQDYQSAKPALDSNLFPMIPDYNLYHHPNDMGS |
| | | IPEHKPFQGMDPIRVNPPPITPLETIKAFKDKQIHPGFGSLPQPPLTLKPIRPRK |
| | | YPNRPSKTPLHERPHACPAEGCDRRFSRSDELTRHLRIHTGHKPFQCRICMR |
| | | SFSRSDHLTTHIRTHTGEKPFACEFCGRKFARSDERKRHAKIHLKQKEKKAE |
| | | KGGAPSASSAPPVSLAPVVTTCA |
| 621 | human | MSTPTDPGAMPHPGPSPGPGPSPGPILGPSPGPGPSPGSVHSMMGPSPGPPSV |
| | SMARCA2 | SHPMPTMGSTDFPQEGMHQMHKPIDGIHDKGIVEDIHCGSMKGTGMRPPHP |
| | | GMGPPQSPMDQHSQGYMSPHPSPLGAPEHVSSPMSGGGPTPPQMPPSQPGA |
| | | LIPGDPQAMSQPNRGPSPFSPVQLHQLRAQILAYKMLARGQPLPETLQLAV |
| | | QGKRTLPGLQQQQQQQQQQQQQQQQQQQQQQQPQQQPPQPQTQQQQQP |
| | | ALVNYNRPSGPGPELSGPSTPQKLPVPAPGGRPSPAPPAAAQPPAAAVPGPS |
| | | VPQPAPGQPSPVLQLQQKQSRISPIQKPQGLDPVEILQEREYRLQARIAHRIQ |
| | | ELENLPGSLPPDLRTKATVELKALRLLNFQRQLRQEVVACMRRDTTLETAL |
| | | NSKAYKRSKRQTLREARMTEKLEKQQKIEQERKRRQKHQEYLNSILQHAK |
| | | DFKEYHRSVAGKIQKLSKAVATWHANTEREQKKETERIEKERMRRLMAED |
| | | EEGYRKLIDQKKDRRLAYLLQQTDEYVANLTNLVWEHKQAQAAKEKKKR |
| | | RRRKKKAEENAEGGESALGPDGEPIDESSQMSDLPVKVTHTETGKVLFGPE |
| | | APKASQLDAWLEMNPGYEVAPRSDSEESDSDYEEEDEEEESSRQETEEKILL |
| | | DPNSEEVSEKDAKQIIETAKQDVDDEYSMQYSARGSQSYYTVAHAISERVE |
| | | KQSALLINGTLKHYQLQGLEWMVSLYNNNLNGILADEMGLGKTIQTIALIT |
| | | YLMEHKRLNGPYLIIVPLSTLSNWTYEFDKWAPSVVKISYKGTPAMRRSLV |
| | | PQLRSGKFNVLLTTYEYIIKDKHILAKIRWKYMIVDEGHRMKNHHCKLTQV |
| | | LNTHYVAPRRILLTGTPLQNKLPELWALLNFLLPTIFKSCSTFEQWFNAPFA |
| | | MTGERVDLNEEETILIIRRLHKVLRPFLLRRLKKEVESQLPEKVEYVIKCDM |
| | | SALQKILYRHMQAKGILLTDGSEKDKKGKGGAKTLMNTIMQLRKICNHPY |
| | | MFQHIEESFAEHLGYSNGVINGAELYRASGKFELLDRILPKLRATNHRVLLF |
| | | CQMTSLMTIMEDYFAFRNFLYLRLDGTTKSEDRAALLKKFNEPGSQYFIFLL |
| | | STRAGGLGLNLQAADTVVIFDSDWNPHQDLQAQDRAHRIGQQNEVRVLRL |
| | | CTVNSVEEKILAAAKYKLNVDQKVIQAGMFDQKSSSHERRAFLQAILEHEE |
| | | ENEEEDEVPDDETLNQMIARREEEFDLFMRMDMDRRREDARNPKRKPRLM |
| | | EEDELPSWIIKDDAEVERLTCEEEEEKIFGRGSRQRRDVDYSDALTEKQWLR |
| | | AIEDGNLEEMEEEVRLKKRKRRRNVDKDPAKEDVEKAKKRRGRPPAEKLS |
| | | PNPPKLTKQMNAIIDTVINYKDRCNVEKVPSNSQLEIEGNSSGRQLSEVFIQL |
| | | PSRKELPEYYELIRKPVDFKKIKERIRNHKYRSLGDLEKDVMLLCHNAQTFN |
| | | LEGSQIYEDSIVLQSVFKSARQKIAKEEESEDESNEEEEEEDEEESESEAKSV |
| | | KVKIKLNKKDDKGRDKGKGKKRPNRGKAKPVVSDFDSDEEQDEREQSEGS |
| | | GTDDE |
| 622 | human | MEPEQMLEGQTQVAENPHSEYGLTDNVERIVENEKINAEKSSKQKVDLQSL |
| | Dpy30 | PTRAYLDQTVVPILLQGLAVLAKERPPNPIEFLASYLLKNKAQFEDRN |
| 623 | human | MSGLGENLDPLASDSRKRKLPCDTPGQGLTCSGEKRRREQESKYIEELAELI |
| | NCOA3 | SANLSDIDNFNVKPDKCAILKETVRQIRQIKEQGKTISNDDDVQKADVSSTG |
| | | QGVIDKDSLGPLLLQALDGFLFVVNRDGNIVFVSENVTQYLQYKQEDLVNT |
| | | SVYNILHEEDRKDFLKNLPKSTVNGVSWTNETQRQKSHTFNCRMLMKTPH |
| | | DILEDINASPEMRQRYETMQCFALSQPRAMMEEGEDLQSCMICVARRITTG |
| | | ERTFPSNPESFITRHDLSGKVVNIDTNSLRSSMRPGFEDIIRRCIQRFFSLNDG |
| | | QSWSQKRHYQEAYLNGHAETPVYRFSLADGTIVTAQTKSKLFRNPVTNDR |
| | | HGFVSTHFLQREQNGYRPNPNPVGQGIRPPMAGCNSSVGGMSMSPNQGLQ |
| | | MPSSRAYGLADPSTTGQMSGARYGGSSNIASLTPGPGMQSPSSYQNNNYGL |
| | | NMSSPPHGSPGLAPNQQNIMISPRNRGSPKIASHQFSPVAGVHSPMASSGNT |
| | | GNHSFSSSSLSALQAISEGVGTSLLSTLSSPGPKLDNSPNMNITQPSKVSNQD |
| | | SKSPLGFYCDQNPVESSMCQSNSRDHLSDKESKESSVEGAENQRGPLESKG |
| | | HKKLLQLLTCSSDDRGHSSLTNSPLDSSCKESSVSVTSPSGVSSSTSGGVSST |
| | | SNMHGSLLQEKHRILHKLLQNGNSPAEVAKITAEATGKDTSSITSCGDGNV |
| | | VKQEQLSPKKKENNALLRYLLDRDDPSDALSKELQPQVEGVDNKMSQCTS |
| | | STIPSSSQEKDPKIKTETSEEGSGDLDNLDAILGDLTSSDFYNNSISSNGSHLG |
| | | TKQQVFQGTNSLGLKSSQSVQSIRPPYNRAVSLDSPVSVGSSPPVKNISAFP |
| | | MLPKQPMLGGNPRMMDSQENYGSSMGGPNRNVTVTQTPSSGDWGLPNSK |
| | | AGRMEPMNSNSMGRPGGDYNTSLPRPALGGSIPTLPLRSNSIPGARPVLQQQ |
| | | QQMLQMRPGEIPMGMGANPYGQAAASNQLGSWPDGMLSMEQVSHGTQN |
| | | RPLLRNSLDDLVGPPSNLEGQSDERALLDQLHTLLSNTDATGLEEIDRALGI |
| | | PELVNQGQALEPKQDAFQGQEAAVMMDQKAGLYGQTYPAQGPPMQGGF |
| | | HLQGQSPSFNSMMNQMNQQGNFPLQGMHPRANIMRPRTNTPKQLRMQLQ |
| | | QRLQGQQFLNQSRQALELKMENPTAGGAAVMRPMMQPQVSSQQGFLNAQ |
| | | MVAQRSRELLSHHFRQQRVAMMMQQQQQQQQQQQQQQQQQQQQQQQQ |
| | | QQQQQTQAFSPPPNVTASPSMDGLLAGPTMPQAPPQQFPYQPNYGMGQQP |
| | | DPAFGRVSSPPNAMMSSRMGPSQNPMMQHPQAASIYQSSEMKGWPSGNLA |
| | | RNSSFSQQQFAHQGNPAVYSMVHMNGSSGHMGQMNMNPMPMSGMPMGP |
| | | DQKYC |
| 624 | human | MRGAASASVREPTPLPGRGAPRTKPRAGRGPTVGTPATLALPARGRPRSRN |
| | ZFP28 | GLASKGQRGAAPTGPGHRALPSRDTALPQERNKKLEAVGTGIEPKAMSQG |
| | | LVTFGDVAVDFSQEEWEWLNPIQRNLYRKVMLENYRNLASLGLCVSKPDV |
| | | ISSLEQGKEPWTVKRKMTRAWCPDLKAVWKIKELPLKKDFCEGKLSQAVIT |
| | | ERLTSYNLEYSLLGEHWDYDALFETQPGLVTIKNLAVDFRQQLHPAQKNFC |
| | | KNGIWENNSDLGSAGHCVAKPDLVSLLEQEKEPWMVKRELTGSLFSGQRS |
| | | VHETQELFPKQDSYAEGVTDRTSNTKLDCSSFRENWDSDYVFGRKLAVGQ |
| | | ETQFRQEPITHNKTLSKERERTYNKSGRWFYLDDSEEKVHNRDSIKNFQKSS |
| | | VVIKQTGIYAGKKLFKCNECKKTFTQSSSLTVHQRIHTGEKPYKCNECGKA |
| | | FSDGSSFARHQRCHTGKKPYECIECGKAFIQNTSLIRHWRYYHTGEKPFDCI |
| | | DCGKAFSDHIGLNQHRRIHTGEKPYKCDVCHKSFRYGSSLTVHQRIHTGEK |
| | | PYECDVCRKAFSHHASLTQHQRVHSGEKPFKCKECGKAFRQNIHLASHLRI |
| | | HTGEKPFECAECGKSFSISSQLATHQRIHTGEKPYECKVCSKAFTQKAHLAQ |
| | | HQKTHTGEKPYECKECGKAFSQTTHLIQHQRVHTGEKPYKCMECGKAFGD |
| | | NSSCTQHQRLHTGQRPYECIECGKAFKTKSSLICHRRSHTGEKPYECSVCGK |
| | | AFSHRQSLSVHQRIHSGKKPYECKECRKTFIQIGHLNQHKRVHTGERSYNY |
| | | KKSRKVFRQTAHLAHHQRIHTGESSTCPSLPSTSNPVDLFPKFLWNPSSLPSP |
| 625 | human | MPTALCPRVLAPKESEEPRKMRSPPGENPSPQGELPSPESSRRLFRRFRYQEA |
| | ZNF496 | AGPREALQRLWDLCGGWLRPERHTKEQILELLVLEQFLAILPREIQSWVRA |
| | | QEPESGEQAVAAVEALEREPGRPWQWLKHCEDPVVIDDGDSPLDQEQEQL |
| | | PVEPHSDLAKNQDAQPITLAQCLGLPSRPPSQLSGDPVLQDAFLLQEENVRD |
| | | TQQVTTLQLPPSRVSPFKDMILCFSEEDWSLLDPAQTGFYGEFIIGEDYGVS |
| | | MPPNDLAAQPDLSQGEENEPRVPELQDLQGKEVPQVSYLDSPSLQPFQVEE |
| | | RRKREELQVPEFQACPQTVVPQNTYPAGGNPRSLENSLDEEVTIEIVLSSSG |
| | | DEDSQHGPYCTEELGSPTEKQRSLPASHRSSTEAGGEVQTSKKSYVCPNCG |
| | | KIFRWRVNFIRHLRSRREQEKPHECSVCGELFSDSEDLDGHLESHEAQKPYR |
| | | CGACGKSFRLNSHLLSHRRIHLQPDRLQPVEKREQAASEDADKGPKEPLEN |
| | | GKAKLSFQCCECGKAFQRHDHLARHRSHFHLKDKARPFQCRYCVKSFTQN |
| | | YDLLRHERLHMKRRSKQALNSY |
| 626 | human | MASMPPTPEAQGPILFEDLAVYFSQEECVTLHPAQRSLSKDGTKESLEDAAL |
| | ZNF597 | MGEEGKPEINQQLSLESMELDELALEKYPIAAPLVPYPEKSSEDGVGNPEAK |
| | | ILSGTPTYKRRVISLLVTIENHTPLVELSEYLGTNTLSEILDSPWEGAKNVYK |
| | | CPECDQNFSDHSYLVLHQKIHSGEKKHKCGDCGKIFNHRANLRTHRRIHTG |
| | | EKPYKCAKCSASFRQHSHLSRHMNSHVKEKPYTCSICGRGFMWLPGLAQH |
| | | QKSHSAENTYESTNCDKHFNEKPNLALPEETFVSGPQYQHTKCMKSFRQSL |
| | | YPALSEKSHDEDSERCSDDGDNFFSFSKFKPLQCPDCDMTFPCFSELISHQNI |
| | | HTEERPHKCKTCEESFALDSELACHQKSHMLAEPFKCTVCGKTFKSNLHLIT |
| | | HKRTHIKNTT |
| 627 | human | MDLPVGPGAAGPSNVPAFLTKLWTLVSDPDTDALICWSPSGNSFHVFDQGQ |
| | HSF1 | FAKEVLPKYFKHNNMASFVRQLNMYGFRKVVHIEQGGLVKPERDDTEFQH |
| | | PCFLRGQEQLLENIKRKVTSVSTLKSEDIKIRQDSVTKLLTDVQLMKGKQEC |
| | | MDSKLLAMKHENEALWREVASLRQKHAQQQKVVNKLIQFLISLVQSNRIL |
| | | GVKRKIPLMLNDSGSAHSMPKYSRQFSLEHVHGSGPYSAPSPAYSSSSLYAP |
| | | DAVASSGPIISDITELAPASPMASPGGSIDERPLSSSPLVRVKEEPPSPPQSPRV |
| | | EEASPGRPSSVDTLLSPTALIDSILRESEPAPASVTALTDARGHTDTEGRPPSP |
| | | PPTSTPEKCLSVACLDNLARTPQMSRVARLFPCPSSSPHGQVQPGNELSDHL |
| | | DAMDSNLDNLQTMLSSHGFSVDTSALLDIQELLSPQEPPRPPEAENSSPDSA |
| | | GALHSAAAVPAGPRLRGHREQRPAGAV |
| 628 | Epstein- | MRPKKDGLEDFLRLTPEIKKQLGSLVSDYCNVLNKEFTAGSVEITLRSYKIC |
| | barr virus | KAFINEAKAHGREWGGLMATLNICNFWAILRNNRVRRRAENAGNDACSIA |
| | strain B95- | CPIVMRYVLDHLIVVTDRFFIQAPSNRVMIPATIGTAMYKLLKHSRVRAYTY |
| | 8 RTA | SKVLGVDRAAIMASGKQVVEHLNRMEKEGLLSSKFKAFCKWVFTYPVLEE |
| | | MFQTMVSSKTGHLTDDVKDVRALIKTLPRASYSSHAGQRSYVSGVLPACLL |
| | | STKSKAVETPILVSGADRMDEELMGNDGGASHTEARYSESGQFHAFTDELE |
| | | SLPSPTMPLKPGAQSADCGDSSSSSSDSGNSDTEQSEREEARAEAPRLRAPK |
| | | SRRTSRPNRGQTPCPSNAAEPEQPWIAAVHQESDERPIFPHPSKPTFLPPVKR |
| | | KKGLRDSREGMFLPKPEAGSAISDVFEGREVCQPKRIRPFHPPGSPWANRPL |
| | | PASLAPTPTGPVHEPVGSLTPAPVPQPLDPAPAVTPEASHLLEDPDEETSQAV |
| | | KALREMADTVIPQKEEAAICGQMDLSHPPPRGHLDELTTTLESMTEDLNLD |
| | | SPLTPELNEILDTFLNDECLLHAMHISTGLSIFDTSLF |
| 629 | ABL1_ | KENLLAGPSENDPNLFVALYDFVASGDNTLSITKGEKLRVLGYNHNGEWC |
| | HUMAN | EAQTKNGQGWVPSNYITPVNSLEKHSWYHG |
| 630 | AF9_ | KSDKQIKNGECDKAYLDELVELHRRLMTLRERHILQQIVNLIEETGHFHITN |
| | HUMAN | TTFDFDLCSLDKTTVRKLQSYLETSGTS |
| 631 | ANM2_ | ECSEAGLLQEGVQPEEFVAIADYAATDETQLSFLRGEKILILRQTTADWWW |
| | HUMAN | GERAGCCGYIPANHVGKHVDEYDPEDTWQ |
| 632 | APBB1_ | GSPSYGSPEDTDSFWNPNAFETDSDLPAGWMRVQDTSGTYYWHIPTGTTQ |
| | HUMAN | WEPPGRASPSQGSSPQEESQLTWTGFAHGE |
| 633 | APC16_ | DLAPPRKALFTYPKGAGEMLEDGSERFLCESVFSYQVASTLKQVKHDQQV |
| | HUMAN | ARMEKLAGLVEELEADEWRFKPIEQLLGFT |
| 634 | BTK_ | PEPAAAPVSTSELKKVVALYDYMPMNANDLQLRKGDEYFILEESNLPWWR |
| | HUMAN | ARDKNGQEGYIPSNYVTEAEDSIEMYEWYS |
| 635 | CACO1_ | SGGEEANLLLPELGSAFYDMASGFTVGTLSETSTGGPATPTWKECPICKERF |
| | HUMAN | PAESDKDALEDHMDGHFFFSTQDPFTFE |
| 636 | CRTC2_ | GPNIILTGDSSPGFSKEIAAALAGVPGFEVSAAGLELGLGLEDELRMEPLGLE |
| | HUMAN | GLNMLSDPCALLPDPAVEESFRSDRLQ |
| 637 | CRTC3_ | NCGSLPNTILPEDSSTSLFKDLNSALAGLPEVSLNVDTPFPLEEELQIEPLSLD |
| | HUMAN | GLNMLSDSSMGLLDPSVEETFRADRL |
| 638 | CXXC1_ | AGEDSKSENGENAPIYCICRKPDINCFMIGCDNCNEWFHGDCIRITEKMAKA |
| | HUMAN | IREWYCRECREKDPKLEIRYRHKKSRER |
| 639 | DPF1_ | PLSLGEDFYREAIEHCRSYNARLCAERSLRLPFLDSQTGVAQNNCYIWMEK |
| | HUMAN | THRGPGLAPGQIYTYPARCWRKKRRLNIL |
| 640 | DPY30_ | EYGLTDNVERIVENEKINAEKSSKQKVDLQSLPTRAYLDQTVVPILLQGLAV |
| | HUMAN | LAKERPPNPIEFLASYLLKNKAQFEDRN |
| 641 | EGR3_ | TVTYLGKFAFDSPSNWCQDNIISLMSAGILGVPPASGALSTQTSTASMVQPP |
| | HUMAN | QGDVEAMYPALPPYSNCGDLYSEPVSFH |
| 642 | ENL_ | SKPEKILKKGTYDKAYTDELVELHRRLMALRERNVLQQIVNLIEETGHFNV |
| | HUMAN | TNTTFDFDLFSLDETTVRKLQSCLEAVAT |
| 643 | FIGN_ | LLVQRTEGFSGLDVAHLCQEAVVGPLHAMPATDLSAIMPSQLRPVTYQDFE |
| | HUMAN | NAFCKIQPSISQKELDMYVEWNKMFGCSQ |
| 644 | FOXO1_ | GGYSSVSSCNGYGRMGLLHQEKLPSDLDGMFIERLDCDMESIIRNDLMDGD |
| | HUMAN | TLDFNFDNVLPNQSFPHSVKTTTHSWVSG |
| 645 | FOXO3_ | DSLSGSSLYSTSANLPVMGHEKFPSDLDLDMFNGSLECDMESIIRSELMDAD |
| | HUMAN | GLDFNFDSLISTQNVVGLNVGNFTGAKQ |
| 646 | IKKA_ | LVGSSLEGAVTPQTSAWLPPTSAEHDHSLSCVVTPQDGETSAQMIEENLNC |
| | HUMAN | LGHLSTIIHEANEEQGNSMMNLDWSWLTE |
| 647 | IMA5_ | RLGEQEAKRNGTGINPYCALIEEAYGLDKIEFLQSHENQEIYQKAFDLIEHYF |
| | HUMAN | GTEDEDSSIAPQVDLNQQQYIFQQCEA |
| 648 | ITCH_ | SGLIIPLTISGGSGPRPLNPVTQAPLPPGWEQRVDQHGRVYYVDHVEKRTT |
| | HUMAN | WDRPEPLPPGWERRVDNMGRIYYVDHFTR |
| 649 | KIBRA_ | PRPELPLPEGWEEARDFDGKVYYIDHTNRTTSWIDPRDRYTKPLTFADCISD |
| | HUMAN | ELPLGWEEAYDPQVGDYFIDHNTKTTQI |
| 650 | KPCI_ | QGHPFFRNVDWDMMEQKQVVPPFKPNISGEFGLDNFDSQFTNEPVQLTPD |
| | HUMAN | DDDIVRKIDQSEFEGFEYINPLLMSAEECV |
| 651 | KS6B2_ | HMNWDDLLAWRVDPPFRPCLQSEEDVSQFDTRFTRQTPVDSPDDTALSESA |
| | HUMAN | NQAFLGFTYVAPSVLDSIKEGFSFQPKLR |
| 652 | MTA3_ | GAVNGAVGTTFQPQNPLLGRACESCYATQSHQWYSWGPPNMQCRLCAIC |
| | HUMAN | WLYWKKYGGLKMPTQSEEEKLSPSPTTEDPR |
| 653 | MYB_ | EAQNVSSHVPYPVALHVNIVNVPQPAAAAIQRHYNDEDPEKEKRIKELELL |
| | HUMAN | LMSTENELKGQQVLPTQNHTCSYPGWHST |
| 654 | MYBA_ | FYIPVQIPGYQYVSPEGNCIEHVQPTSAFIQQPFIDEDPDKEKKIKELEMLLM |
| | HUMAN | SAENEVRRKRIPSQPGSFSSWSGSFLM |
| 655 | NCOA2_ | PFGSSPDDLLCPHPAAESPSDEGALLDQLYLALRNFDGLEEIDRALGIPELVS |
| | HUMAN | QSQAVDPEQFSSQDSNIMLEQKAPVFP |
| 656 | NCOA3_ | LRNSLDDLVGPPSNLEGQSDERALLDQLHTLLSNTDATGLEEIDRALGIPEL |
| | HUMAN | VNQGQALEPKQDAFQGQEAAVMMDQKAG |
| 657 | NOTC1_ | LCHILDYSFGGGAGRDIPPPLIEEACELPECQEDAGNKVCSLQCNNHACGW |
| | HUMAN | DGGDCSLNFNDPWKNCTQSLQCWKYFSDG |
| 658 | NOTC1_ | LQCNNHACGWDGGDCSLNFNDPWKNCTQSLQCWKYFSDGHCDSQCNSA |
| | HUMAN | GCLFDGFDCQRAEGQCNPLYDQYCKDHFSDGH |
| 659 | NOTC2_ | EACNSHACQWDGGDCSLTMENPWANCSSPLPCWDYINNQCDELCNTVEC |
| | HUMAN | LFDNFECQGNSKTCKYDKYCADHFKDNHCDQ |
| 660 | PRP19_ | TNKILTGGADKNVVVFDKSSEQILATLKGHTKKVTSVVFHPSQDLVFSASP |
| | HUMAN | DATIRIWSVPNASCVQVVRAHESAVTGLS |
| 661 | PYGO1_ | RHGHSSSDPVYPCGICTNEVNDDQDAILCEASCQKWFHRICTGMTETAYGL |
| | HUMAN | LTAEASAVWGCDTCMADKDVQLMRTRETF |
| 662 | PYGO2_ | SGPQPPPGLVYPCGACRSEVNDDQDAILCEASCQKWFHRECTGMTESAYGL |
| | HUMAN | LTTEASAVWACDLCLKTKEIQSVYIREGM |
| 663 | SAV1_ | HASGIGRVAATSLGNLTNHGSEDLPLPPGWSVDWTMRGRKYYIDHNTNTT |
| | HUMAN | HWSHPLEREGLPPGWERVESSEFGTYYVDH |
| 664 | SMCA2_ | SQPGALIPGDPQAMSQPNRGPSPFSPVQLHQLRAQILAYKMLARGQPLPETL |
| | HUMAN | QLAVQGKRTLPGLQQQQQ |
| 665 | SMRC2_ | MYTKKNVPSKSKAAASATREWTEQETLLLLEALEMYKDDWNKVSEHVGS |
| | HUMAN | RTQDECILHFLRLPIEDPYLEDSEASLGPLA |
| 666 | STAT2_ | SQTVPEPDQGPVSQPVPEPDLPCDLRHLNTEPMEIFRNCVKIEEIMPNGDPLL |
| | HUMAN | AGQNTVDEVYVSRPSHFYTDGPLMPSD |
| 667 | T2EB_ | SSGYKFGVLAKIVNYMKTRHQRGDTHPLTLDEILDETQHLDIGLKQKQWL |
| | HUMAN | MTEALVNNPKIEVIDGKYAFKPKYNVRDKK |
| 668 | U2AF4_ | VEVQEHYDSFFEEVFTELQEKYGEIEEMNVCDNLGDHLVGNVYVKFRREE |
| | HUMAN | DGERAVAELSNRWFNGQAVHGELSPVTDFR |
| 669 | WBP4_ | YYDLISGASQWEKPEGFQGDLKKTAVKTVWVEGLSEDGFTYYYNTETGES |
| | HUMAN | RWEKPDDFIPHTSDLPSSKVNENSLGTLDE |
| 670 | WWP1_ | AMQQFNQRYLYSASMLAAENDPYGPLPPGWEKRVDSTDRVYFVNHNTKT |
| | HUMAN | TQWEDPRTQGLQNEEPLPEGWEIRYTREGVR |
| 671 | WWP2_ | AMQHFSQRFLYQSSSASTDHDPLGPLPPGWEKRQDNGRVYYVNHNTRTTQ |
| | HUMAN | WEDPRTQGMIQEPALPPGWEMKYTSEGVRY |
| 672 | WWTR1_ | GAAGSPAQQHAHLRQQSYDVTDELPLPPGWEMTFTATGQRYFLNHIEKITT |
| | HUMAN | WQDPRKAMNQPLNHMNLHPAVSSTPVPQR |
| 673 | ZFP28_ | LEYSLLGEHWDYDALFETQPGLVTIKNLAVDFRQQLHPAQKNFCKNGIWE |
| | HUMAN | NNSDLGSAGHCVAKPDLVSLLEQEKEPWMV |
| 674 | ZN473_ | AEEFVTLKDVGMDFTLGDWEQLGLEQGDTFWDTALDNCQDLFLLDPPRPN |
| | HUMAN | LTSHPDGSEDLEPLAGGSPEATSPDVTETK |
| 675 | ZN496_ | QEENVRDTQQVTTLQLPPSRVSPFKDMILCFSEEDWSLLDPAQTGFYGEFIIG |
| | HUMAN | EDYGVSMPPNDLAAQPDLSQGEENEPR |
| 676 | ZN597_ | ASMPPTPEAQGPILFEDLAVYFSQEECVTLHPAQRSLSKDGTKESLEDAALM |
| | HUMAN | GEEGKPEINQQLSLESMELDELALEKYP |
| 677 | p300 | MAENVVEPGPPSAKRPKLSSPALSASASDGTDFGSLFDLEHDLPDELINSTEL |
| | | GLTNGGDINQLQTSLGMVQDAASKHKQLSELLRSGSSPNLNMGVGGPGQV |
| | | MASQAQQSSPGLGLINSMVKSPMTQAGLTSPNMGMGTSGPNQGPTQSTGM |
| | | MNSPVNQPAMGMNTGMNAGMNPGMLAAGNGQGIMPNQVMNGSIGAGR |
| | | GRQNMQYPNPGMGSAGNLLTEPLQQGSPQMGGQTGLRGPQPLKMGMMN |
| | | NPNPYGSPYTQNPGQQIGASGLGLQIQTKTVLSNNLSPFAMDKKAVPGGGM |
| | | PNMGQQPAPQVQQPGLVTPVAQGMGSGAHTADPEKRKLIQQQLVLLLHA |
| | | HKCQRREQANGEVRQCNLPHCRTMKNVLNHMTHCQSGKSCQVAHCASSR |
| | | QIISHWKNCTRHDCPVCLPLKNAGDKRNQQPILTGAPVGLGNPSSLGVGQQ |
| | | SAPNLSTVSQIDPSSIERAYAALGLPYQVNQMPTQPQVQAKNQQNQQPGQS |
| | | PQGMRPMSNMSASPMGVNGGVGVQTPSLLSDSMLHSAINSQNPMMSENAS |
| | | VPSLGPMPTAAQPSTTGIRKQWHEDITQDLRNHLVHKLVQAIFPTPDPAALK |
| | | DRRMENLVAYARKVEGDMYESANNRAEYYHLLAEKIYKIQKELEEKRRTR |
| | | LQKQNMLPNAAGMVPVSMNPGPNMGQPQPGMTSNGPLPDPSMIRGSVPN |
| | | QMMPRITPQSGLNQFGQMSMAQPPIVPRQTPPLQHHGQLAQPGALNPPMG |
| | | YGPRMQQPSNQGQFLPQTQFPSQGMNVTNIPLAPSSGQAPVSQAQMSSSSC |
| | | PVNSPIMPPGSQGSHIHCPQLPQPALHQNSPSPVPSRTPTPHHTPPSIGAQQPP |
| | | ATTIPAPVPTPPAMPPGPQSQALHPPPRQTPTPPTTQLPQQVQPSLPAAPSAD |
| | | QPQQQPRSQQSTAASVPTPTAPLLPPQPATPLSQPAVSIEGQVSNPPSTSSTE |
| | | VNSQAIAEKQPSQEVKMEAKMEVDQPEPADTQPEDISESKVEDCKMESTET |
| | | EERSTELKTEIKEEEDQPSTSATQSSPAPGQSKKKIFKPEELRQALMPTLEAL |
| | | YRQDPESLPFRQPVDPQLLGIPDYFDIVKSPMDLSTIKRKLDTGQYQEPWQY |
| | | VDDIWLMFNNAWLYNRKTSRVYKYCSKLSEVFEQEIDPVMQSLGYCCGRK |
| | | LEFSPQTLCCYGKQLCTIPRDATYYSYQNRYHFCEKCFNEIQGESVSLGDDP |
| | | SQPQTTINKEQFSKRKNDTLDPELFVECTECGRKMHQICVLHHEIIWPAGFV |
| | | CDGCLKKSARTRKENKFSAKRLPSTRLGTFLENRVNDFLRRQNHPESGEVT |
| | | VRVVHASDKTVEVKPGMKARFVDSGEMAESFPYRTKALFAFEEIDGVDLC |
| | | FFGMHVQEYGSDCPPPNQRRVYISYLDSVHFFRPKCLRTAVYHEILIGYLEY |
| | | VKKLGYTTGHIWACPPSEGDDYIFHCHPPDQKIPKPKRLQEWYKKMLDKA |
| | | VSERIVHDYKDIFKQATEDRLTSAKELPYFEGDFWPNVLEESIKELEQEEEE |
| | | RKREENTSNESTDVTKGDSKNAKKKNNKKTSKNKSSLSRGNKKKPGMPNV |
| | | SNDLSQKLYATMEKHKEVFFVIRLIAGPAANSLPPIVDPDPLIPCDLMDGRD |
| | | AFLTLARDKHLEFSSLRRAQWSTMCMLVELHTQSQDRFVYTCNECKHHVE |
| | | TRWHCTVCEDYDLCITCYNTKNHDHKMEKLGLGLDDESNNQQAAATQSP |
| | | GDSRRLSIQRCIQSLVHACQCRNANCSLPSCQKMKRVVQHTKGCKRKTNG |
| | | GCPICKQLIALCCYHAKHCQENKCPVPFCLNIKQKLRQQQLQHRLQQAQML |
| | | RRRMASMQRTGVVGQQQGLPSPTPATPTTPTGQQPTTPQTPQPTSQPQPTPP |
| | | NSMPPYLPRTQAAGPVSQGKAAGQVTPPTPPQTAQPPLPGPPPAAVEMAM |
| | | QIQRAAETQRQMAHVQIFQRPIQHQMPPMTPMAPMGMNPPPMTRGPSGHL |
| | | EPGMGPTGMQQQPPWSQGGLPQPQQLQSGMPRPAMMSVAQHGQPLNMA |
| | | PQPGLGQVGISPLKPGTVSQQALQNLLRTLRSPSSPLQQQQVLSILHANPQLL |
| | | AAFIKQRAAKYANSNPQPIPGQPGMPQGQPGLQPPTMPGQQGVHSNPAMQ |
| | | NMNPMQAGVQRAGLPQQQPQQQLQPPMGGMSPQAQQMNMNHNTMPSQF |
| | | RDILRRQQMMQQQQQQGAGPGIGPGMANHNQFQQPQGVGYPPQQQQRM |
| | | QHHMQQMQQGNMGQIGQLPQALGAEAGASLQAYQQRLLQQQMGSPVQP |
| | | NPMSPQQHMLPNQAQSPHLQGQQIPNSLSNQVRSPQPVPSPRPQSQPPHSSP |
| | | SPRMQPQPSPHHVSPQTSSPHPGLVAAQANPMEQGHFASPDQNSMLSQLAS |
| | | NPGMANLHGASATDLGLSTDNSDLNSNLSQSTLDIH |
| 678 | CREBBP | MAENLLDGPPNPKRAKLSSPGFSANDSTDFGSLFDLENDLPDELIPNGGELG |
| | | LLNSGNLVPDAASKHKQLSELLRGGSGSSINPGIGNVSASSPVQQGLGGQA |
| | | QGQPNSANMASLSAMGKSPLSQGDSSAPSLPKQAASTSGPTPAASQALNPQ |
| | | AQKQVGLATSSPATSQTGPGICMNANFNQTHPGLLNSNSGHSLINQASQGQ |
| | | AQVMNGSLGAAGRGRGAGMPYPTPAMQGASSSVLAETLTQVSPQMTGHA |
| | | GLNTAQAGGMAKMGITGNTSPFGQPFSQAGGQPMGATGVNPQLASKQSM |
| | | VNSLPTFPTDIKNTSVTNVPNMSQMQTSVGIVPTQAIATGPTADPEKRKLIQ |
| | | QQLVLLLHAHKCQRREQANGEVRACSLPHCRTMKNVLNHMTHCQAGKAC |
| | | QVAHCASSRQIISHWKNCTRHDCPVCLPLKNASDKRNQQTILGSPASGIQNT |
| | | IGSVGTGQQNATSLSNPNPIDPSSMQRAYAALGLPYMNQPQTQLQPQVPGQ |
| | | QPAQPQTHQQMRTLNPLGNNPMNIPAGGITTDQQPPNLISESALPTSLGATN |
| | | PLMNDGSNSGNIGTLSTIPTAAPPSSTGVRKGWHEHVTQDLRSHLVHKLVQ |
| | | AIFPTPDPAALKDRRMENLVAYAKKVEGDMYESANSRDEYYHLLAEKIYKI |
| | | QKELEEKRRSRLHKQGILGNQPALPAPGAQPPVIPQAQPVRPPNGPLSLPVN |
| | | RMQVSQGMNSFNPMSLGNVQLPQAPMGPRAASPMNHSVQMNSMGSVPG |
| | | MAISPSRMPQPPNMMGAHTNNMMAQAPAQSQFLPQNQFPSSSGAMSVGM |
| | | GQPPAQTGVSQGQVPGAALPNPLNMLGPQASQLPCPPVTQSPLHPTPPPAST |
| | | AAGMPSLQHTTPPGMTPPQPAAPTQPSTPVSSSGQTPTPTPGSVPSATQTQST |
| | | PTVQAAAQAQVTPQPQTPVQPPSVATPQSSQQQPTPVHAQPPGTPLSQAAA |
| | | SIDNRVPTPSSVASAETNSQQPGPDVPVLEMKTETQAEDTEPDPGESKGEPR |
| | | SEMMEEDLQGASQVKEETDIAEQKSEPMEVDEKKPEVKVEVKEEEESSSNG |
| | | TASQSTSPSQPRKKIFKPEELRQALMPTLEALYRQDPESLPFRQPVDPQLLGI |
| | | PDYFDIVKNPMDLSTIKRKLDTGQYQEPWQYVDDVWLMFNNAWLYNRKT |
| | | SRVYKFCSKLAEVFEQEIDPVMQSLGYCCGRKYEFSPQTLCCYGKQLCTIPR |
| | | DAAYYSYQNRYHFCEKCFTEIQGENVTLGDDPSQPQTTISKDQFEKKKNDT |
| | | LDPEPFVDCKECGRKMHQICVLHYDIIWPSGFVCDNCLKKTGRPRKENKFS |
| | | AKRLQTTRLGNHLEDRVNKFLRRQNHPEAGEVFVRVVASSDKTVEVKPGM |
| | | KSRFVDSGEMSESFPYRTKALFAFEEIDGVDVCFFGMHVQEYGSDCPPPNT |
| | | RRVYISYLDSIHFFRPRCLRTAVYHEILIGYLEYVKKLGYVTGHIWACPPSEG |
| | | DDYIFHCHPPDQKIPKPKRLQEWYKKMLDKAFAERIIHDYKDIFKQATEDR |
| | | LTSAKELPYFEGDFWPNVLEESIKELEQEEEERKKEESTAASETTEGSQGDS |
| | | KNAKKKNNKKTNKNKSSISRANKKKPSMPNVSNDLSQKLYATMEKHKEV |
| | | FFVIHLHAGPVINTLPPIVDPDPLLSCDLMDGRDAFLTLARDKHWEFSSLRR |
| | | SKWSTLCMLVELHTQGQDRFVYTCNECKHHVETRWHCTVCEDYDLCINC |
| | | YNTKSHAHKMVKWGLGLDDEGSSQGEPQSKSPQESRRLSIQRCIQSLVHAC |
| | | QCRNANCSLPSCQKMKRVVQHTKGCKRKTNGGCPVCKQLIALCCYHAKH |
| | | CQENKCPVPFCLNIKHKLRQQQIQHRLQQAQLMRRRMATMNTRNVPQQSL |
| | | PSPTSAPPGTPTQQPSTPQTPQPPAQPQPSPVSMSPAGFPSVARTQPPTTVSTG |
| | | KPTSQVPAPPPPAQPPPAAVEAARQIEREAQQQQHLYRVNINNSMPPGRTG |
| | | MGTPGSQMAPVSLNVPRPNQVSGPVMPSMPPGQWQQAPLPQQQPMPGLPR |
| | | PVISMQAQAAVAGPRMPSVQPPRSISPSALQDLLRTLKSPSSPQQQQQVLNI |
| | | LKSNPQLMAAFIKQRTAKYVANQPGMQPQPGLQSQPGMQPQPGMHQQPSL |
| | | QNLNAMQAGVPRPGVPPQQQAMGGLNPQGQALNIMNPGHNPNMASMNP |
| | | QYREMLRRQLLQQQQQQQQQQQQQQQQQQGSAGMAGGMAGHGQFQQP |
| | | QGPGGYPPAMQQQQRMQQHLPLQGSSMGQMAAQMGQLGQMGQPGLGA |
| | | DSTPNIQQALQQRILQQQQMKQQIGSPGQPNPMSPQQHMLSGQPQASHLPG |
| | | QQIATSLSNQVRSPAPVQSPRPQSQPPHSSPSPRIQPQPSPHHVSPQTGSPHPG |
| | | LAVTMASSIDQGHLGNPEQSAMLPQLNTPSRSALSSELSLVGDTTGDTLEKF |
| | | VEGL |
| 679 | linker | SGSETPGTSESATPES |
| 680 | linker | SGGS |
| 681 | linker | SGGSSGSETPGTSESATPESSGGS |
| 682 | linker | SGGSSGGSSGSETPGTSESATPESSGGSSGGS |
| 683 | linker | GGSGGSPGSPAGSPTSTEEGTSESATPESGPGTSTEPSEGSAPGSPAGSPTSTE |
| | | EGTSTEPSEGSAPGTSTEPSEGSAPGTSESATPESGPGSEPATSGGSGGS |
| 684 | XTEN | SGSETPGTSESATPES |
| | linker | |
| 685 | XTEN | SGGSSGGSSGSETPGTSESATPES |
| | linker | |
| 686 | XTEN | SGGSSGGSSGSETPGTSESATPESSGGSSGGSSGGSSGGS |
| | linker | |
| 687 | XTEN | SGGSSGGSSGSETPGTSESATPESSGGSSGGSSGGSSGGSSGSETPGTSESATP |
| | linker | ESSGGSSGGS |
| 688 | XTEN | PGSPAGSPTSTEEGTSESATPESGPGTSTEPSEGSAPGSPAGSPTSTEEGTSTEP |
| | linker | SEGSAPGTSTEPSEGSAPGTSESATPESGPGSEPATS |
| 689 | NLS | PKKKRKV |
| 690 | NLS | AVKRPAATKKAGQAKKKKLD |
| 691 | NLS | MSRRRKANPTKLSENAKKLAKEVEN |
| 692 | NLS | PAAKRVKLD |
| 693 | NLS | KLKIKRPVK |
| 694 | NLS | MDSLLMNRRKFLYQFKNVRWAKGRRETYLC |
| 695 | overlapping | GTACGTCGTCTTTAGGTTGGGGGGAGGGGTTTTATGCGATGGAGTTTC |
| | binding | CCCACACTGAGTGGGTGGAGACTGAAGTTAGGCCAGCTTGGCACTTGA |
| | sites | TGTAATTCTCCTTGGAATTTGCCCTTTTTGAGTTTGGATCTTGGTTCATTC |
| | | TCAAGCCTCAGACAGTGGTTCAAAGTTTTTTTCTTCCATTTCAGGTGTCG |
| | | TGACGCTAGCGCTACCGGTCGCCACCATGGTGAGCAAGGGCGCCGAGCT |
| | | GTTCACCGGCATCGTGCCCATCCTGATCGAGCTGAATGGCGATGTGAA |
| | | TGGCCACAAGTTCAGCGTGAGCGGCGAGGGCGAGGGCGATGCCACCTA |
| | | CGGCAAGCTGACCCTGAAGTTCATCTGCACCACCGGCAAGCTGCCTGTG |
| | | CCCTGGCCC |
| 696 | GFP-1 | TAGGTTgGGGGGAGGGGTT |
| | target | |
| | binding site | |
| 697 | GFP-2 | GTGGGTGGAGACtGAAGTT |
| | target | |
| | binding site | |
| 698 | GFP-3 | TGGGCAcGATGCCGGTGAA |
| | target | |
| | binding site | |
| 699 | GFP-4 | GGCGAGGGCGAGGGCGAT |
| | target | |
| | binding site | |
| 700 | GFP-5 | GAGGGCGAGGGCGATGCC |
| | target | |
| | binding site | |
| 701 | GFP-6 | GCCGGTGGTGCAGATGAA |
| | target | |
| | binding site | |
| 702 | GFP-7 | GCAGCTtGCCGGTGGTGCA |
| | target | |
| | binding site | |
| 703 | Exemplary | SRPGERPFQCRICMRNFS[F1]HTRTHTGEKPFQCRICMRNFS[F2]HLRTH[linker1] |
| | Zinc Finger | FQCRICMRNFS[F3]HTRTHTGEKPFQCRICMRNFS[F4]HLRTH[linker2] |
| | Sequence | FQCRICMRNFS[F5]HTRTHTGEKPFQCRICMRNFS[F6]HLRTHLRGS |
| 704 | linker | TGSQKP |
| 705 | linker | TGGGGSQKP |

| | SEQ ID | F1 | SEQ ID | F2 | SEQ ID | F3 |
|---|---|---|---|---|---|---|
| Description | NO | Sequence | NO | Sequence | NO | Sequence |
| GFP1-ZF1 | 716 | HKSSLTR | 757 | RTEHLAR | 798 | QSAHLKR |
| GFP1-ZF2 | 717 | HKSSLTR | 758 | RTEHLAR | 799 | TSAHLAR |
| GFP1-ZF3 | 718 | IKAILTR | 759 | RREHLVR | 800 | QSAHLKR |
| GFP1-ZF4 | 719 | IKAILTR | 760 | RREHLVR | 801 | TSAHLAR |
| GFP2-ZF1 | 720 | TSTLLNR | 761 | QQTNLTR | 802 | DEANLRR |
| GFP2-ZF2 | 721 | TSTLLNR | 762 | QQTNLTR | 803 | DEANLRR |
| GFP2-ZF3 | 722 | TSTLLNR | 763 | QQTNLTR | 804 | DRGNLTR |
| GFP2-ZF4 | 723 | TSTLLNR | 764 | QQTNLTR | 805 | DRGNLTR |
| GFP2-ZF5 | 724 | HKSSLTR | 765 | QTNNLGR | 806 | DEANLRR |
| GFP2-ZF6 | 725 | HKSSLTR | 766 | QTNNLGR | 807 | DEANLRR |
| GFP2-ZF7 | 726 | HKSSLTR | 767 | QTNNLGR | 808 | DRGNLTR |
| GFP2-ZF8 | 727 | HKSSLTR | 768 | QTNNLGR | 809 | DRGNLTR |
| GFP3-ZF1 | 728 | QQTNLTR | 769 | IRHHLKR | 810 | DSSVLRR |
| GFP3-ZF2 | 729 | QQTNLTR | 770 | IRHHLKR | 811 | DGSTLNR |
| GFP3-ZF3 | 730 | RKPNLLR | 771 | EAHHLSR | 812 | DSSVLRR |
| GFP3-ZF4 | 731 | RKPNLLR | 772 | EAHHLSR | 813 | DGSTLNR |
| GFP4-ZF1 | 732 | VRHNLTR | 773 | ESGHLKR | 814 | RQDNLGR |
| GFP5-ZF1 | 733 | DSSVLRR | 774 | LSTNLTR | 815 | LKEHLTR |
| GFP5-ZF2 | 734 | DSSVLRR | 775 | LSTNLTR | 816 | LKEHLTR |
| GFP5-ZF3 | 735 | DSSVLRR | 776 | LSTNLTR | 817 | SPSKLVR |
| GFP5-ZF4 | 736 | DSSVLRR | 777 | LSTNLTR | 818 | SPSKLVR |
| GFP5-ZF5 | 737 | DGSTLNR | 778 | VRHNLTR | 819 | LKEHLTR |
| GFP5-ZF6 | 738 | DGSTLNR | 779 | VRHNLTR | 820 | LKEHLTR |
| GFP5-ZF7 | 739 | DGSTLNR | 780 | VRHNLTR | 821 | SPSKLVR |
| GFP5-ZF8 | 740 | DGSTLNR | 781 | VRHNLTR | 822 | SPSKLVR |
| GFP6-ZF1 | 741 | RKPNLLR | 782 | VRHNLTR | 823 | DKAQLGR |
| GFP6-ZF2 | 742 | RKPNLLR | 783 | VRHNLTR | 824 | DKAQLGR |
| GFP6-ZF3 | 743 | RKPNLLR | 784 | VRHNLTR | 825 | QSTTLKR |
| GFP6-ZF4 | 744 | RKPNLLR | 785 | VRHNLTR | 826 | QSTTLKR |
| GFP6-ZF5 | 745 | QQTNLTR | 786 | VGSNLTR | 827 | DKAQLGR |
| GFP6-ZF6 | 746 | QQTNLTR | 787 | VGSNLTR | 828 | DKAQLGR |
| GFP6-ZF7 | 747 | QQTNLTR | 788 | VGSNLTR | 829 | QSTTLKR |
| GFP6-ZF8 | 748 | QQTNLTR | 789 | VGSNLTR | 830 | QSTTLKR |
| GFP7-ZF1 | 749 | QSTTLKR | 790 | VDHHLRR | 831 | EAHHLSR |
| GFP7-ZF2 | 750 | QSTTLKR | 791 | VDHHLRR | 832 | EAHHLSR |
| GFP7-ZF3 | 751 | QSTTLKR | 792 | VDHHLRR | 833 | RQSRLQR |
| GFP7-ZF4 | 752 | QSTTLKR | 793 | VDHHLRR | 834 | RQSRLQR |
| GFP7-ZF5 | 753 | DKAQLGR | 794 | EAHHLSR | 835 | EAHHLSR |
| GFP7-ZF6 | 754 | DKAQLGR | 795 | EAHHLSR | 836 | EAHHLSR |
| GFP7-ZF7 | 755 | DKAQLGR | 796 | EAHHLSR | 837 | RQSRLQR |
| GFP7-ZF8 | 756 | DKAQLGR | 797 | EAHHLSR | 838 | RQSRLQR |
| GFP1-ZF1 | 839 | RTEHLAR | 880 | HKSSLTR | 921 | RPESLAP |
| GFP1-ZF2 | 840 | RREHLVR | 881 | HKSSLTR | 922 | RPESLAP |
| GFP1-ZF3 | 841 | RTEHLAR | 882 | HKSSLTR | 923 | RPESLAP |
| GFP1-ZF4 | 842 | RREHLVR | 883 | HKSSLTR | 924 | RPESLAP |
| GFP2-ZF1 | 843 | QSAHLKR | 884 | IPNKLAR | 925 | RREVLEN |
| GFP2-ZF2 | 844 | QSAHLKR | 885 | EAHHLSR | 926 | RKDALHV |
| GFP2-ZF3 | 845 | QGGHLKR | 886 | IPNKLAR | 927 | RREVLEN |
| GFP2-ZF4 | 846 | QGGHLKR | 887 | EAHHLSR | 928 | RKDALHV |
| GFP2-ZF5 | 847 | QSAHLKR | 888 | IPNKLAR | 929 | RREVLEN |
| GFP2-ZF6 | 848 | QSAHLKR | 889 | EAHHLSR | 930 | RKDALHV |
| GFP2-ZF7 | 849 | QGGHLKR | 890 | IPNKLAR | 931 | RREVLEN |
| GFP2-ZF8 | 850 | QGGHLKR | 891 | EAHHLSR | 932 | RKDALHV |
| GFP3-ZF1 | 851 | LSTNLTR | 892 | QSTTLKR | 933 | RSDHLSL |
| GFP3-ZF2 | 852 | VRHNLTR | 893 | QSTTLKR | 934 | RSDHLSL |
| GFP3-ZF3 | 853 | LSTNLTR | 894 | QSTTLKR | 935 | RSDHLSL |
| GFP3-ZF4 | 854 | VRHNLTR | 895 | QSTTLKR | 936 | RSDHLSL |
| GFP4-ZF1 | 855 | KNHSLNN | 896 | RQDNLGR | 937 | KNHSLNN |
| GFP5-ZF1 | 856 | RVDNLPR | 897 | LKEHLTR | 938 | RVDNLPR |
| GFP5-ZF2 | 857 | RVDNLPR | 898 | SPSKLVR | 939 | RQDNLGR |
| GFP5-ZF3 | 858 | RQDNLGR | 899 | LKEHLTR | 940 | RVDNLPR |
| GFP5-ZF4 | 859 | RQDNLGR | 900 | SPSKLVR | 941 | RQDNLGR |
| GFP5-ZF5 | 860 | RVDNLPR | 901 | LKEHLTR | 942 | RVDNLPR |
| GFP5-ZF6 | 861 | RVDNLPR | 902 | SPSKLVR | 943 | RQDNLGR |
| GFP5-ZF7 | 862 | RQDNLGR | 903 | LKEHLTR | 944 | RVDNLPR |
| GFP5-ZF8 | 863 | RQDNLGR | 904 | SPSKLVR | 945 | RQDNLGR |
| GFP6-ZF1 | 864 | EAHHLSR | 905 | RQSRLQR | 946 | KGDHLRR |
| GFP6-ZF2 | 865 | EAHHLSR | 906 | EAHHLSR | 947 | DPSNLRR |
| GFP6-ZF3 | 866 | VDHHLRR | 907 | RQSRLQR | 948 | KGDHLRR |
| GFP6-ZF4 | 867 | VDHHLRR | 908 | EAHHLSR | 949 | DPSNLRR |
| GFP6-ZF5 | 868 | EAHHLSR | 909 | RQSRLQR | 950 | KGDHLRR |
| GFP6-ZF6 | 869 | EAHHLSR | 910 | EAHHLSR | 951 | DPSNLRR |
| GFP6-ZF7 | 870 | VDHHLRR | 911 | RQSRLQR | 952 | KGDHLRR |
| GFP6-ZF8 | 871 | VDHHLRR | 912 | EAHHLSR | 953 | DPSNLRR |
| GFP7-ZF1 | 872 | DPSNLRR | 913 | QRSDLTR | 954 | QGGTLRR |
| GFP7-ZF2 | 873 | DPSNLRR | 914 | TKQILGR | 955 | QSTTLKR |
| GFP7-ZF3 | 874 | DSSVLRR | 915 | QRSDLTR | 956 | QGGTLRR |
| GFP7-ZF4 | 875 | DSSVLRR | 916 | TKQILGR | 957 | QSTTLKR |
| GFP7-ZF5 | 876 | DPSNLRR | 917 | QRSDLTR | 958 | QGGTLRR |
| GFP7-ZF6 | 877 | DPSNLRR | 918 | TKQILGR | 959 | QSTTLKR |
| GFP7-ZF7 | 878 | DSSVLRR | 919 | QRSDLTR | 960 | QGGTLRR |
| GFP7-ZF8 | 879 | DSSVLRR | 920 | TKQILGR | 961 | QSTTLKR |

| SEQ | | |
|---|---|---|
| ID NO | Description | Sequence |
| 962 | SPACER | GCCTACCGCAGGATGTTCGG |
| 963 | SPACER | GGCCCGGGGACGAGGCGTAG |
| 964 | SPACER | GCGCACGGCAGAGGAGCGCG |
| 965 | SPACER | GCCCTCGTTCGCCTCTTCTC |
| 966 | TRACR | GTTTAAGAGCTAAGCTGGAAACAGCATAGCAAGTTTAAATAAGG |
| | | CTAGTCCGTTATCAACTTGAAAAAGTGGCACCGAGTCGGTGCTTT |
| | | TTTT |
| 967 | TRACR | GTTTAAGAGCTAAGCTGGAAACAGCATAGCAAGTTTAAATAAGG |
| | | CTAGTCCGTTATCAACTTGAAAAAGTGGCACCGAGTCGGTGCTTT |
| | | TTTT |
| 968 | TRACR | GTTTAAGAGCTAAGCTGGAAACAGCATAGCAAGTTTAAATAAGG |
| | | CTAGTCCGTTATCAACTTGAAAAAGTGGCACCGAGTCGGTGCTTT |
| | | TTTT |
| 969 | TRACR | GTTTAAGAGCTAAGCTGGAAACAGCATAGCAAGTTTAAATAAGG |
| | | CTAGTCCGTTATCAACTTGAAAAAGTGGCACCGAGTCGGTGCTTT |
| | | TTTT |
| 970 | SPACER | GTGTCCAGGGACAATGAGCA |
| 971 | SPACER | GCGGCCCGGAGCCTACGAGG |
| 972 | SPACER | GCGGCGGCGGCAGCAGCTGCG |
| 973 | SPACER | GCCGGACTCGGACGCGTGGT |
| 974 | TRACR | GTTTAAGAGCTAAGCTGGAAACAGCATAGCAAGTTTAAATAAGG |
| | | CTAGTCCGTTATCAACTTGAAAAAGTGGCACCGAGTCGGTGCTTT |
| | | TTTT |
| 975 | TRACR | GTTTAAGAGCTAAGCTGGAAACAGCATAGCAAGTTTAAATAAGG |
| | | CTAGTCCGTTATCAACTTGAAAAAGTGGCACCGAGTCGGTGCTTT |
| | | TTTT |
| 976 | TRACR | GTTTAAGAGCTAAGCTGGAAACAGCATAGCAAGTTTAAATAAGG |
| | | CTAGTCCGTTATCAACTTGAAAAAGTGGCACCGAGTCGGTGCTTT |
| | | TTTT |
| 977 | TRACR | GTTTAAGAGCTAAGCTGGAAACAGCATAGCAAGTTTAAATAAGG |
| | | CTAGTCCGTTATCAACTTGAAAAAGTGGCACCGAGTCGGTGCTTT |
| | | TTTT |
| 978 | DNMT3A- | MAPKKKRKMNHDQEFDPPKVYPPVPAEKRKPIRVLSLFDGIATGLLVL |
| | 3L-ZF- | KDLGIQVDRYIASEVCEDSITVGMVRHQGKIMYVGDVRSVTQKHIQEWG |
| | KRAB (ZF | PFDLVIGGSPCNDLSIVNPARKGLYEGTGRLFFEFYRLLHDARPKEGDDR |
| | is GFP1- | PFFWLFENVVAMGVSDKRDISRFLESNPVMIDAKEVSAAHRARYFWGNL |
| | ZF1) | PGMNRPLASTVNDKLELQECLEHGRIAKFSKVRTITTRSNSIKQGKDQHF |
| | | PVFMNEKEDILWCTEMERVFGFPVHYTDVSNMSRLARQRLLGRSWSVPV |
| | | IRHLFAPLKEYFACVSSGNSNANSRGPSFSSGLVPLSLRGSHMGPME |
| | | IYKTVSAWKRQPVRVLSLFRNIDKVLKSLGFLESGSGSGGGTLK |
| | | YVEDVTNVVRRDVEKWGPFDLVYGSTQPLGSSCDRCPGWYMFQ |
| | | FHRILQYALPRQESQRPFFWIFMDNLLLTEDDQETTTRFLQTEAV |
| | | TLQDVRGRDYQNAMRVWSNIPGLKSKHAPLTPKEEEYLQAQVR |
| | | SRSKLDAPKVDLLVKNCLLPLREYFKYFSQNSLPLSGGGGSGGGG |
| | | SVGIHGVPSRPGERPFQCRICMRNFSHKSSLTRHTRTHTGEKPFQCRI |
| | | CMRNFSRTEHLARHLRTHTGSQKPFQCRICMRNFSQSAHLKRHTRT |
| | | HTGEKPFQCRICMRNFSRTEHLARHLRTHTGGGGSQKPFQCRICMRN |
| | | FSHKSSLTRHTRTHTGEKPFQCRICMRNFSRPESLAPHLRTHLRGSGG |
| | | GSMDAKSLTAWSRTLVTFKDVFVDFTREEWKLLDTAQQIVYRNVM |
| | | LENYKNLVSLGYQLTKPDVILRLEKGEEPWLVEREIHQETHPDSETA |
| | | FEIKSSV |
| 979 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | ZIM3 | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GMNNSQGRVTFEDVTVNFTQGEWQRLNPEQRNLYRDVMLENYSNL |
| | | VSVGQGETTKPDVILRLEQGKEPWLEEEEVLGSGRAEKNGDIGGQIW |
| | | KPKDVKESL |
| 980 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | HP1b | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GMGKKQNKKKVEEVLEEEEEEYVVEKVLDRRVVKGKVEYLLKWK |
| | | GFSDEDNTWEPEENLDCPDLIAEFLQSQKTAHETDKSEGGKRKADSD |
| | | SEDKGEESKPKKKKEESEKPRGFARGLEPERIIGATDSSGELMFLMK |
| | | WKNSDEADLVPAKEANVKCPQVVISFYEERLTWHSYPSEDDDKKD |
| | | DKN |
| 981 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | RYBP | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GPSEANSIQSANATTKTSETNHTSRPRLKNVDRSTAQQLAVTVGNVT |
| | | VIITDFKEKTRSSSTSSSTVTSSAGSEQQNQSSS |
| 982 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | ZFP28 | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GNKKLEAVGTGIEPKAMSQGLVTFGDVAVDFSQEEWEWLNPIQRNL |
| | | YRKVMLENYRNLASLGLCVSKPDVISSLEQGKEPW |
| 983 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | ZN627 | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GDSVAFEDVAVNFTLEEWALLDPSQKNLYRDVMRETFRNLASVGK |
| | | QWEDQNIEDPFKIPRRNISHIPERLCESKEGGQGEE |
| 984 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | CDYL2 | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GASGDLYEVERIVDKRKNKKGKWEYLIRWKGYGSTEDTWEPEHHL |
| | | LHCEEFIDEFNGLHMSKDKRIKSGKQSSTSKLLRDS |
| 985 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | TOX | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GKDPNEPQKPVSAYALFFRDTQAAIKGQNPNATFGEVSKIVASMWD |
| | | GLGEEQKQVYKKKTEAAKKEYLKQLAAYRASLVSK |
| | | MNHDQEFDPPKVYPPVPAEKRKPIRVLSLFDGIATGLLVLKDLGIQV |
| | | DRYIASEVCEDSITVGMVRHQGKIMYVGDVRSVTQKHIQEWGPFDL |
| | | VIGGSPCNDLSIVNPARKGLYEGTGRLFFEFYRLLHDARPKEGDDRPF |
| | | FWLFENVVAMGVSDKRDISRFLESNPVMIDAKEVSAAHRARYFWG |
| 986 | DNMT3A/ | NLPGMNRPLASTVNDKLELQECLEHGRIAKFSKVRTITTRSNSIKQGK |
| | L- | DQHFPVFMNEKEDILWCTEMERVFGFPVHYTDVSNMSRLARQRLLG |
| | dSpCas9- | RSWSVPVIRHLFAPLKEYFACVSSGNSNANSRGPSFSSGLVPLSLRGS |
| | XTEN16- | HMGPMEIYKTVSAWKRQPVRVLSLFRNIDKVLKSLGFLESGSGSGG |
| | SCMH1 | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GDASRLSGRDPSSWTVEDVMQFVREADPQLGPHADLFRKHEIDGKA |
| | | LLLLRSDMMMKYMGLKLGPALKLSYHIDRLKQGKF |
| 987 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | SCML2 | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GKQGFSKDPSTWSVDEVIQFMKHTDPQISGPLADLFRQHEIDGKALF |
| | | LLKSDVMMKYMGLKLGPALKLCYYIEKLKEGKYS |
| 988 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | CBX8 | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GGSGPPSSGGGLYRDMGAQGGRPSLIARIPVARILGDPEEESWSPSLT |
| | | NLEKVVVTDVTSNFLTVTIKESNTDQGFFKEKR |
| 989 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | TOX3 | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GKDPNEPQKPVSAYALFFRDTQAAIKGQNPNATFGEVSKIVASMWD |
| | | SLGEEQKQVYKRKTEAAKKEYLKALAAYRASLVSK |
| 990 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | TOX4 | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GKDPNEPQKPVSAYALFFRDTQAAIKGQNPNATFGEVSKIVASMWD |
| | | SLGEEQKQVYKRKTEAAKKEYLKALAAYKDNQECQ |
| 991 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | I2BP1 | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GASVQASRRQWCYLCDLPKMPWAMVWDFSEAVCRGCVNFEGADR |
| | | IELLIDAARQLKRSHVLPEGRSPGPPALKHPATKDLA |
| | | MNHDQEFDPPKVYPPVPAEKRKPIRVLSLFDGIATGLLVLKDLGIQV |
| | | DRYIASEVCEDSITVGMVRHQGKIMYVGDVRSVTQKHIQEWGPFDL |
| | | VIGGSPCNDLSIVNPARKGLYEGTGRLFFEFYRLLHDARPKEGDDRPF |
| | | FWLFENVVAMGVSDKRDISRFLESNPVMIDAKEVSAAHRARYFWG |
| 992 | DNMT3A/ | NLPGMNRPLASTVNDKLELQECLEHGRIAKFSKVRTITTRSNSIKQGK |
| | L- | DQHFPVFMNEKEDILWCTEMERVFGFPVHYTDVSNMSRLARQRLLG |
| | dSpCas9- | RSWSVPVIRHLFAPLKEYFACVSSGNSNANSRGPSFSSGLVPLSLRGS |
| | XTEN16- | HMGPMEIYKTVSAWKRQPVRVLSLFRNIDKVLKSLGFLESGSGSGG |
| | MBD2 | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GMRAHPGGGRCCPEQEEGESAAGGSGAGGDSAIEQGGQGSALAPSP |
| | | VSGVRREGARGGGRGRGRWKQAGRGGGVCGRGRGRGRGRGRGRG |
| | | RGRGRGRPPSGGSGLGGDGGGCGGGGSGGGGAPRREPVPFPSGSAG |
| | | PGPRGPRATESGKRMDCPALPPGWKKEEVIRKSGLSAGKSDVYYFSP |
| | | SGKKFRSKPQLARYLGNTVDLSSFDFRTGKMMPSKLQKNKQRLRND |
| | | PLNQNKGKPDLNTTLPIRQTASIFKQPVTKVTNHPSNKVKSDPQRMN |
| | | EQPRQLFWEKRLQGLSASDVTEQIIKTMELPKGLQGVGPGSNDETLL |
| | | SAVASALHTSSAPITGQVSAAVEKNPAVWLNTSQPLCKAFIVTDEDI |
| | | RKQEERVQQVRKKLEEALMADILSRAADTEEMDIEMDSGDEA |
| 993 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | SetDB1 | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GMSSLPGCIGLDAATATVESEEIAELQQAVVEELGISMEELRHFIDEE |
| | | LEKMDCVQQRKKQLAELETWVIQKESEVAHVDQLFDDASRAVINC |
| | | ESLVKDFYSKLGLQYRDSSSEDESSRPTEIIEIPDEDDDVLSIDSGDAG |
| | | SRTPKDQKLREAMAALRKSAQDVQKFMDAVNKKSSSQDLHKGTLS |
| | | QMSGELSKDGDLIVSMRILGKKRTKTWHKGTLIAIQTVGPGKKYKV |
| | | KFDNKGKSLLSGNHIAYDYHPPADKLYVGSRVVAKYKDGNQVWLY |
| | | AGIVAETPNVKNKLRFLIFFDDGYASYVTQSELYPICRPLKKTWEDIE |
| | | DISCRDFIEEYVTAYPNRPMVLLKSGQLIKTEWEGTWWKSRVEEVD |
| | | GSLVRILFLDDKRCEWIYRGSTRLEPMFSMKTSSASALEKKQGQLRT |
| | | RPNMGAVRSKGPVVQYTQDLTGTGTQFKPVEPPQPTAPPAPPFPPAP |
| | | PLSPQAGDSDLESQLAQSRKQVAKKSTSFRPGSVGSGHSSPTSPALSE |
| | | NVSGGKPGINQTYRSPLGSTASAPAPSALPAPPAPPVFHGMLERAPAE |
| | | PSYRAPMEKLFYLPHVCSYTCLSRVRPMRNEQYRGKNPLLVPLLYD |
| | | FRRMTARRRVNRKMGFHVIYKTPCGLCLRTMQEIERYLFETGCDFLF |
| | | LEMFCLDPYVLVDRKFQPYKPFYYILDITYGKEDVPLSCVNEIDTTPP |
| | | PQVAYSKERIPGKGVFINTGPEFLVGCDCKDGCRDKSKCACHQLTIQ |
| | | ATACTPGGQINPNSGYQYKRLEECLPTGVYECNKRCKCDPNMCTNR |
| | | LVQHGLQVRLQLFKTQNKGWGIRCLDDIAKGSFVCIYAGKILTDDFA |
| | | DKEGLEMGDEYFANLDHIESVENFKEGYESDAPCSSDSSGVDLKDQ |
| | | EDGNSGTEDPEESNDDSSDDNFCKDEDFSTSSVWRSYATRRQTRGQ |
| | | KENGLSETTSKDSHPPDLGPPHIPVPPSIPVGGCNPPSSEETPKNKVAS |
| | | WLSCNSVSEGGFADSDSHSSFKTNEGGEGRAGGSRMEAEKASTSGL |
| | | GIKDEGDIKQAKKEDTDDRNKMSVVTESSRNYGYNPSPVKPEGLRR |
| | | PPSKTSMHQSRRLMASAQSNPDDVLTLSSSTESEGESGTSRKPTAGQ |
| | | TSATAVDSDDIQTISSGSEGDDFEDKKNMTGPMKRQVAVKSTRGFA |
| | | LKSTHGIAIKSTNMASVDKGESAPVRKNTRQFYDGEESCYIIDAKLE |
| | | GNLGRYLNHSCSPNLFVQNVFVDTHDLRFPWVAFFASKRIRAGTELT |
| | | WDYNYEVGSVEGKELLCCCGAIECRGRLL |
| 994 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | MeCP2 | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GMVAGMLGLREEKSEDQDLQGLKDKPLKFKKVKKDKKEEKEGKH |
| | | EPVQPSAHHSAEPAEAGKAETSEGSGSAPAVPEASASPKQRRSIIRDR |
| | | GPMYDDPTLPEGWTRKLKQRKSGRSAGKYDVYLINPQGKAFRSKVE |
| | | LIAYFEKVGDTSLDPNDFDFTVTGRGSPSRREQKPPKKPKSPKAPGTG |
| | | RGRGRPKGSGTTRPKAATSEGVQVKRVLEKSPGKLLVKMPFQTSPG |
| | | GKAEGGGATTSTQVMVIKRPGRKRKAEADPQAIPKKRGRKPGSVVA |
| | | AAAAEAKKKAVKESSIRSVQETVLPIKKRKTRETVSIEVKEVVKPLL |
| | | VSTLGEKSGKGLKTCKSPGRKSKESSPKGRSSSASSPPKKEHHHHHH |
| | | HSESPKAPVPLLPPLPPPPPEPESSEDPTSPPEPQDLSSSVCKEEKMPRG |
| | | GSLESDGCPKEPAKTQPAVATAATAAEKYKHRGEGERKDIVSSSMP |
| | | RPNREEPVDSRTPVTERVS |
| 995 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | Kap1 | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GMAASAAAASAAAASAASGSPGPGEGSAGGEKRSTAPSAAASASAS |
| | | AAASSPAGGGAEALELLEHCGVCRERLRPEREPRLLPCLHSACSACL |
| | | GPAAPAAANSSGDGGAAGDGTVVDCPVCKQQCFSKDIVENYFMRD |
| | | SGSKAATDAQDANQCCTSCEDNAPATSYCVECSEPLCETCVEAHQR |
| | | VKYTKDHTVRSTGPAKSRDGERTVYCNVHKHEPLVLFCESCDTLTC |
| | | RDCQLNAHKDHQYQFLEDAVRNQRKLLASLVKRLGDKHATLQKST |
| | | KEVRSSIRQVSDVQKRVQVDVKMAILQIMKELNKRGRVLVNDAQK |
| | | VTEGQQERLERQHWTMTKIQKHQEHILRFASWALESDNNTALLLSK |
| | | KLIYFQLHRALKMIVDPVEPHGEMKFQWDLNAWTKSAEAFGKIVAE |
| | | RPGTNSTGPAPMAPPRAPGPLSKQGSGSSQPMEVQEGYGFGSGDDP |
| | | YSSAEPHVSGVKRSRSGEGEVSGLMRKVPRVSLERLDLDLTADSQPP |
| | | VFKVFPGSTTEDYNLIVIERGAAAAATGQPGTAPAGTPGAPPLAGMA |
| | | IVKEEETEAAIGAPPTATEGPETKPVLMALAEGPGAEGPRLASPSGST |
| | | SSGLEVVAPEGTSAPGGGPGTLDDSATICRVCQKPGDLVMCNQCEFC |
| | | FHLDCHLPALQDVPGEEWSCSLCHVLPDLKEEDGSLSLDGADSTGV |
| | | VAKLSPANQRKCERVLLALFCHEPCRPLHQLATDSTFSLDQPGGTLD |
| | | LTLIRARLQEKLSPPYSSPQEFAQDVGRMFKQFNKLTEDKADVQSIIG |
| | | LQRFFETRMNEAFGDTKFSAVLVEPPPMSLPGAGLSSQELSGGPGDG |
| | | P |
| 996 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | HP1a | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GMGKKTKRTADSSSSEDEEEYVVEKVLDRRVVKGQVEYLLKWKGF |
| | | SEEHNTWEPEKNLDCPELISEFMKKYKKMKEGENNKPREKSESNKR |
| | | KSNFSNSADDIKSKKKREQSNDIARGFERGLEPEKIIGATDSCGDLMF |
| | | LMKWKGTDEADLVLAKEANVKCPQIVIAFYEERLTWHAYPEDAEN |
| | | KEKETAKS |
| 997 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | EED | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GMSEREVSTAPAGTDMPAAKKQKLSSDENSNPDLSGDENDDAVSIE |
| | | SGTNTERPDTPTNTPNAPGRKSWGKGKWKSKKCKYSFKCVNSLKED |
| | | HNQPLFGVQFNWHSKEGDPLVFATVGSNRVTLYECHSQGEIRLLQS |
| | | YVDADADENFYTCAWTYDSNTSHPLLAVAGSRGIIRIINPITMQCIKH |
| | | YVGHGNAINELKFHPRDPNLLLSVSKDHALRLWNIQTDTLVAIFGGV |
| | | EGHRDEVLSADYDLLGEKIMSCGMDHSLKLWRINSKRMMNAIKESY |
| | | DYNPNKTNRPFISQKIHFPDFSTRDIHRNYVDCVRWLGDLILSKSCEN |
| | | AIVCWKPGKMEDDIDKIKPSESNVTILGRFDYSQCDIWYMRFSMDF |
| | | WQKMLALGNQVGKLYVWDLEVEDPHKAKCTTLTHHKCGAAIRQT |
| | | SFSRDSSILIAVCDDASIWRWDRLR |
| 998 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | RBBP4 | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GMADKEAAFDDAVEERVINEEYKIWKKNTPFLYDLVMTHALEWPS |
| | | LTAQWLPDVTRPEGKDFSIHRLVLGTHTSDEQNHLVIASVQLPNDDA |
| | | QFDASHYDSEKGEFGGFGSVSGKIEIEIKINHEGEVNRARYMPQNPCII |
| | | ATKTPSSDVLVFDYTKHPSKPDPSGECNPDLRLRGHQKEGYGLSWN |
| | | PNLSGHLLSASDDHTICLWDISAVPKEGKVVDAKTIFTGHTAVVEDV |
| | | SWHLLHESLFGSVADDQKLMIWDTRSNNTSKPSHSVDAHTAEVNCL |
| | | SFNPYSEFILATGSADKTVALWDLRNLKLKLHSFESHKDEIFQVQWS |
| | | PHNETILASSGTDRRLNVWDLSKIGEEQSPEDAEDGPPELLFIHGGHT |
| | | AKISDFSWNPNEPWVICSVSEDNIMQVWQMAENIYNDEDPEGSVDP |
| | | EGQGS |
| 999 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | RCOR1 | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GMPAMVEKGPEVSGKRRGRNNAAASASAAAASAAASAACASPAAT |
| | | AASGAAASSASAAAASAAAAPNNGQNKSLAAAAPNGNSSSNSWEE |
| | | GSSGSSSDEEHGGGGMRVGPQYQAVVPDFDPAKLARRSQERDNLG |
| | | MLVWSPNQNLSEAKLDEYIAIAKEKHGYNMEQALGMLFWHKHNIE |
| | | KSLADLPNFTPFPDEWTVEDKVLFEQAFSFHGKTFHRIQQMLPDKSI |
| | | ASLVKFYYSWKKTRTKTSVMDRHARKQKREREESEDELEEANGNN |
| | | PIDIEVDQNKESKKEVPPTETVPQVKKEKHSTQAKNRAKRKPPKGMF |
| | | LSQEDVEAVSANATAATTVLRQLDMELVSVKRQIQNIKQTNSALKE |
| | | KLDGGIEPYRLPEVIQKCNARWTTEEQLLAVQAIRKYGRDFQAISDVI |
| | | GNKSVVQVKNFFVNYRRRFNIDEVLQEWEAEHGKEETNGPSNQKPV |
| | | KSPDNSIKMPEEEDEAPVLDVRYASAS |
| 1000 | DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | EZH2 | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GMGQTGKKSEKGPVCWRKRVKSEYMRLRQLKRFRRADEVKSMFSS |
| | | NRQKILERTEILNQEWKQRRIQPVHILTSVSSLRGTRECSVTSDLDFPT |
| | | QVIPLKTLNAVASVPIMYSWSPLQQNFMVEDETVLHNIPYMGDEVL |
| | | DQDGTFIEELIKNYDGKVHGDRECGFINDEIFVELVNALGQYNDDDD |
| | | DDDGDDPEEREEKQKDLEDHRDDKESRPPRKFPSDKIFEAISSMFPD |
| | | KGTAEELKEKYKELTEQQLPGALPPECTPNIDGPNAKSVQREQSLHS |
| | | FHTLFCRRCFKYDCFLHPFHATPNTYKRKNTETALDNKPCGPQCYQ |
| | | HLEGAKEFAAALTAERIKTPPKRPGGRRRGRLPNNSSRPSTPTINVLE |
| | | SKDTDSDREAGTETGGENNDKEEEEKKDETSSSSEANSRCQTPIKMK |
| | | PNIEPPENVEWSGAEASMFRVLIGTYYDNFCAIARLIGTKTCRQVYEF |
| | | RVKESSIIAPAPAEDVDTPPRKKKRKHRLWAAHCRKIQLKKDGSSNH |
| | | VYNYQPCDHPRQPCDSSCPCVIAQNFCEKFCQCSSECQNRFPGCRCK |
| | | AQCNTKQCPCYLAVRECDPDLCLTCGAADHWDSKNVSCKNCSIQR |
| | | GSKKHLLLAPSDVAGWGIFIKDPVQKNEFISEYCGEIISQDEADRRGK |
| | | VYDKYMCSFLFNLNNDFVVDATRKGNKIRFANHSVNPNCYAKVMM |
| | | VNGDHRIGIFAKRAIQTGEELFFDYRYSQADALKYVGIEREMEIP |
| 1001 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB-ZIM3 | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGMNNSQGRVTF |
| | | EDVTVNFTQGEWQRLNPEQRNLYRDVMLENYSNLVSVGQGETTKP |
| | | DVILRLEQGKEPWLEEEEVLGSGRAEKNGDIGGQIWKPKDVKESL |
| 1002 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB-ZFP28 | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGNKKLEAVGTGI |
| | | EPKAMSQGLVTFGDVAVDFSQEEWEWLNPIQRNLYRKVMLENYRN |
| | | LASLGLCVSKPDVISSLEQGKEPW |
| 1003 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB-ZN627 | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGDSVAFEDVAV |
| | | NFTLEEWALLDPSQKNLYRDVMRETFRNLASVGKQWEDQNIEDPFK |
| | | IPRRNISHIPERLCESKEGGQGEE |
| 1004 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB-RYBP | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGPSEANSIQSAN |
| | | ATTKTSETNHTSRPRLKNVDRSTAQQLAVTVGNVTVIITDFKEKTRS |
| | | SSTSSSTVTSSAGSEQQNQSSS |
| 1005 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB- | DQHFPVFMNEKEDILWCTEMERVFGFPVHYTDVSNMSRLARQRLLG |
| | CDYL2 | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGASGDLYEVERI |
| | | VDKRKNKKGKWEYLIRWKGYGSTEDTWEPEHHLLHCEEFIDEFNGL |
| | | HMSKDKRIKSGKQSSTSKLLRDS |
| 1006 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB-TOX | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGKDPNEPQKPVS |
| | | AYALFFRDTQAAIKGQNPNATFGEVSKIVASMWDGLGEEQKQVYK |
| | | KKTEAAKKEYLKQLAAYRASLVSK |
| 1007 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB- | DQHFPVFMNEKEDILWCTEMERVFGFPVHYTDVSNMSRLARQRLLG |
| | SCMH1 | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGDASRLSGRDPS |
| | | SWTVEDVMQFVREADPQLGPHADLFRKHEIDGKALLLLRSDMMMK |
| | | YMGLKLGPALKLSYHIDRLKQGKF |
| 1008 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB- | DQHFPVFMNEKEDILWCTEMERVFGFPVHYTDVSNMSRLARQRLLG |
| | SCML2 | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGKQGFSKDPST |
| | | WSVDEVIQFMKHTDPQISGPLADLFRQHEIDGKALFLLKSDVMMKY |
| | | MGLKLGPALKLCYYIEKLKEGKYS |
| 1009 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB-CBX8 | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGGSGPPSSGGGL |
| | | YRDMGAQGGRPSLIARIPVARILGDPEEESWSPSLTNLEKVVVTDVT |
| | | SNFLTVTIKESNTDQGFFKEKR |
| 1010 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB-TOX3 | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGKDPNEPQKPVS |
| | | AYALFFRDTQAAIKGQNPNATFGEVSKIVASMWDSLGEEQKQVYKR |
| | | KTEAAKKEYLKALAAYRASLVSK |
| 1011 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB-TOX4 | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGKDPNEPQKPVS |
| | | AYALFFRDTQAAIKGQNPNATFGEVSKIVASMWDSLGEEQKQVYKR |
| | | KTEAAKKEYLKALAAYKDNQECQ |
| 1012 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB-I2BP1 | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGASVQASRRQW |
| | | CYLCDLPKMPWAMVWDFSEAVCRGCVNFEGADRIELLIDAARQLK |
| | | RSHVLPEGRSPGPPALKHPATKDLA |
| 1013 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB-MBD2 | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGMRAHPGGGRC |
| | | CPEQEEGESAAGGSGAGGDSAIEQGGQGSALAPSPVSGVRREGARG |
| | | GGRGRGRWKQAGRGGGVCGRGRGRGRGRGRGRGRGRGRGRPPSG |
| | | GSGLGGDGGGCGGGGSGGGGAPRREPVPFPSGSAGPGPRGPRATES |
| | | GKRMDCPALPPGWKKEEVIRKSGLSAGKSDVYYFSPSGKKFRSKPQ |
| | | LARYLGNTVDLSSFDFRTGKMMPSKLQKNKQRLRNDPLNQNKGKP |
| | | DLNTTLPIRQTASIFKQPVTKVTNHPSNKVKSDPQRMNEQPRQLFWE |
| | | KRLQGLSASDVTEQIIKTMELPKGLQGVGPGSNDETLLSAVASALHT |
| | | SSAPITGQVSAAVEKNPAVWLNTSQPLCKAFIVTDEDIRKQEERVQQ |
| | | VRKKLEEALMADILSRAADTEEMDIEMDSGDEA |
| 1014 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB- | DQHFPVFMNEKEDILWCTEMERVFGFPVHYTDVSNMSRLARQRLLG |
| | MeCP2 | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGMVAGMLGLRE |
| | | EKSEDQDLQGLKDKPLKFKKVKKDKKEEKEGKHEPVQPSAHHSAEP |
| | | AEAGKAETSEGSGSAPAVPEASASPKQRRSIIRDRGPMYDDPTLPEG |
| | | WTRKLKQRKSGRSAGKYDVYLINPQGKAFRSKVELIAYFEKVGDTS |
| | | LDPNDFDFTVTGRGSPSRREQKPPKKPKSPKAPGTGRGRGRPKGSGT |
| | | TRPKAATSEGVQVKRVLEKSPGKLLVKMPFQTSPGGKAEGGGATTS |
| | | TQVMVIKRPGRKRKAEADPQAIPKKRGRKPGSVVAAAAAEAKKKA |
| | | VKESSIRSVQETVLPIKKRKTRETVSIEVKEVVKPLLVSTLGEKSGKG |
| | | LKTCKSPGRKSKESSPKGRSSSASSPPKKEHHHHHHHSESPKAPVPLL |
| | | PPLPPPPPEPESSEDPTSPPEPQDLSSSVCKEEKMPRGGSLESDGCPKE |
| | | PAKTQPAVATAATAAEKYKHRGEGERKDIVSSSMPRPNREEPVDSR |
| | | TPVTERVS |
| 1015 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB-Kap1 | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGMAASAAAASA |
| | | AAASAASGSPGPGEGSAGGEKRSTAPSAAASASASAAASSPAGGGA |
| | | EALELLEHCGVCRERLRPEREPRLLPCLHSACSACLGPAAPAAANSS |
| | | GDGGAAGDGTVVDCPVCKQQCFSKDIVENYFMRDSGSKAATDAQD |
| | | ANQCCTSCEDNAPATSYCVECSEPLCETCVEAHQRVKYTKDHTVRS |
| | | TGPAKSRDGERTVYCNVHKHEPLVLFCESCDTLTCRDCQLNAHKDH |
| | | QYQFLEDAVRNQRKLLASLVKRLGDKHATLQKSTKEVRSSIRQVSD |
| | | VQKRVQVDVKMAILQIMKELNKRGRVLVNDAQKVTEGQQERLERQ |
| | | HWTMTKIQKHQEHILRFASWALESDNNTALLLSKKLIYFQLHRALK |
| | | MIVDPVEPHGEMKFQWDLNAWTKSAEAFGKIVAERPGTNSTGPAP |
| | | MAPPRAPGPLSKQGSGSSQPMEVQEGYGFGSGDDPYSSAEPHVSGV |
| | | KRSRSGEGEVSGLMRKVPRVSLERLDLDLTADSQPPVFKVFPGSTTE |
| | | DYNLIVIERGAAAAATGQPGTAPAGTPGAPPLAGMAIVKEEETEAAI |
| | | GAPPTATEGPETKPVLMALAEGPGAEGPRLASPSGSTSSGLEVVAPE |
| | | GTSAPGGGPGTLDDSATICRVCQKPGDLVMCNQCEFCFHLDCHLPA |
| | | LQDVPGEEWSCSLCHVLPDLKEEDGSLSLDGADSTGVVAKLSPANQ |
| | | RKCERVLLALFCHEPCRPLHQLATDSTFSLDQPGGTLDLTLIRARLQE |
| | | KLSPPYSSPQEFAQDVGRMFKQFNKLTEDKADVQSIIGLQRFFETRM |
| | | NEAFGDTKFSAVLVEPPPMSLPGAGLSSQELSGGPGDGP |
| 1016 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB-HP1a | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGMGKKTKRTAD |
| | | SSSSEDEEEYVVEKVLDRRVVKGQVEYLLKWKGFSEEHNTWEPEKN |
| | | LDCPELISEFMKKYKKMKEGENNKPREKSESNKRKSNFSNSADDIKS |
| | | KKKREQSNDIARGFERGLEPEKIIGATDSCGDLMFLMKWKGTDEAD |
| | | LVLAKEANVKCPQIVIAFYEERLTWHAYPEDAENKEKETAKS |
| 1017 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB-HP1b | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGMGKKQNKKK |
| | | VEEVLEEEEEEYVVEKVLDRRVVKGKVEYLLKWKGFSDEDNTWEP |
| | | EENLDCPDLIAEFLQSQKTAHETDKSEGGKRKADSDSEDKGEESKPK |
| | | KKKEESEKPRGFARGLEPERIIGATDSSGELMFLMKWKNSDEADLVP |
| | | AKEANVKCPQVVISFYEERLTWHSYPSEDDDKKDDKN |
| 1018 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB-EED | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGMSEREVSTAPA |
| | | GTDMPAAKKQKLSSDENSNPDLSGDENDDAVSIESGTNTERPDTPTN |
| | | TPNAPGRKSWGKGKWKSKKCKYSFKCVNSLKEDHNQPLFGVQFNW |
| | | HSKEGDPLVFATVGSNRVTLYECHSQGEIRLLQSYVDADADENFYT |
| | | CAWTYDSNTSHPLLAVAGSRGIIRIINPITMQCIKHYVGHGNAINELK |
| | | FHPRDPNLLLSVSKDHALRLWNIQTDTLVAIFGGVEGHRDEVLSADY |
| | | DLLGEKIMSCGMDHSLKLWRINSKRMMNAIKESYDYNPNKTNRPFI |
| | | SQKIHFPDFSTRDIHRNYVDCVRWLGDLILSKSCENAIVCWKPGKME |
| | | DDIDKIKPSESNVTILGRFDYSQCDIWYMRFSMDFWQKMLALGNQV |
| | | GKLYVWDLEVEDPHKAKCTTLTHHKCGAAIRQTSFSRDSSILIAVCD |
| | | DASIWRWDRLR |
| 1019 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB- | DQHFPVFMNEKEDILWCTEMERVFGFPVHYTDVSNMSRLARQRLLG |
| | RBBP4 | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGMADKEAAFDD |
| | | AVEERVINEEYKIWKKNTPFLYDLVMTHALEWPSLTAQWLPDVTRP |
| | | EGKDFSIHRLVLGTHTSDEQNHLVIASVQLPNDDAQFDASHYDSEKG |
| | | EFGGFGSVSGKIEIEIKINHEGEVNRARYMPQNPCIIATKTPSSDVLVF |
| | | DYTKHPSKPDPSGECNPDLRLRGHQKEGYGLSWNPNLSGHLLSASD |
| | | DHTICLWDISAVPKEGKVVDAKTIFTGHTAVVEDVSWHLLHESLFGS |
| | | VADDQKLMIWDTRSNNTSKPSHSVDAHTAEVNCLSFNPYSEFILATG |
| | | SADKTVALWDLRNLKLKLHSFESHKDEIFQVQWSPHNETILASSGTD |
| | | RRLNVWDLSKIGEEQSPEDAEDGPPELLFIHGGHTAKISDFSWNPNEP |
| | | WVICSVSEDNIMQVWQMAENIYNDEDPEGSVDPEGQGS |
| 1020 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB- | DQHFPVFMNEKEDILWCTEMERVFGFPVHYTDVSNMSRLARQRLLG |
| | RCOR1 | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGMPAMVEKGPE |
| | | VSGKRRGRNNAAASASAAAASAAASAACASPAATAASGAAASSAS |
| | | AAAASAAAAPNNGQNKSLAAAAPNGNSSSNSWEEGSSGSSSDEEHG |
| | | GGGMRVGPQYQAVVPDFDPAKLARRSQERDNLGMLVWSPNQNLSE |
| | | AKLDEYIAIAKEKHGYNMEQALGMLFWHKHNIEKSLADLPNFTPFP |
| | | DEWTVEDKVLFEQAFSFHGKTFHRIQQMLPDKSIASLVKFYYSWKK |
| | | TRTKTSVMDRHARKQKREREESEDELEEANGNNPIDIEVDQNKESK |
| | | KEVPPTETVPQVKKEKHSTQAKNRAKRKPPKGMFLSQEDVEAVSAN |
| | | ATAATTVLRQLDMELVSVKRQIQNIKQTNSALKEKLDGGIEPYRLPE |
| | | VIQKCNARWTTEEQLLAVQAIRKYGRDFQAISDVIGNKSVVQVKNFF |
| | | VNYRRRFNIDEVLQEWEAEHGKEETNGPSNQKPVKSPDNSIKMPEEE |
| | | DEAPVLDVRYASAS |
| 1021 | :DNMT3A/ | MNHDQEFDPPKVYPPVPAEKRKPIRVLSLFDGIATGLLVLKDLGIQV |
| | L- | DRYIASEVCEDSITVGMVRHQGKIMYVGDVRSVTQKHIQEWGPFDL |
| | dSpCas9- | VIGGSPCNDLSIVNPARKGLYEGTGRLFFEFYRLLHDARPKEGDDRPF |
| | XTEN16- | FWLFENVVAMGVSDKRDISRFLESNPVMIDAKEVSAAHRARYFWG |
| | KOX1KR | NLPGMNRPLASTVNDKLELQECLEHGRIAKFSKVRTITTRSNSIKQGK |
| | AB-EZH2 | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HMGPMEIYKTVSAWKRQPVRVLSLFRNIDKVLKSLGFLESGSGSGG |
| | | GTLKYVEDVTNVVRRDVEKWGPFDLVYGSTQPLGSSCDRCPGWYM |
| | | FQFHRILQYALPRQESQRPFFWIFMDNLLLTEDDQETTTRFLQTEAVT |
| | | LQDVRGRDYQNAMRVWSNIPGLKSKHAPLTPKEEEYLQAQVRSRSK |
| | | LDAPKVDLLVKNCLLPLREYFKYFSQNSLPLGGPSSGAPPPSGGSPAG |
| | | SPTSTEEGTSESATPESGPGTSTEPSEGSAPGSPAGSPTSTEEGTSTEPS |
| | | EGSAPGTSTEPSEPKKKRKVYMDKKYSIGLAIGTNSVGWAVITDEYK |
| | | VPSKKFKVLGNTDRHSIKKNLIGALLFDSGETAEATRLKRTARRRYT |
| | | RRKNRICYLQEIFSNEMAKVDDSFFHRLEESFLVEEDKKHERHPIFGN |
| | | IVDEVAYHEKYPTIYHLRKKLVDSTDKADLRLIYLALAHMIKFRGHF |
| | | LIEGDLNPDNSDVDKLFIQLVQTYNQLFEENPINASGVDAKAILSARL |
| | | SKSRRLENLIAQLPGEKKNGLFGNLIALSLGLTPNFKSNFDLAEDAKL |
| | | QLSKDTYDDDLDNLLAQIGDQYADLFLAAKNLSDAILLSDILRVNTE |
| | | ITKAPLSASMIKRYDEHHQDLTLLKALVRQQLPEKYKEIFFDQSKNG |
| | | YAGYIDGGASQEEFYKFIKPILEKMDGTEELLVKLNREDLLRKQRTF |
| | | DNGSIPHQIHLGELHAILRRQEDFYPFLKDNREKIEKILTFRIPYYVGP |
| | | LARGNSRFAWMTRKSEETITPWNFEEVVDKGASAQSFIERMTNFDK |
| | | NLPNEKVLPKHSLLYEYFTVYNELTKVKYVTEGMRKPAFLSGEQKK |
| | | AIVDLLFKTNRKVTVKQLKEDYFKKIECFDSVEISGVEDRFNASLGT |
| | | YHDLLKIIKDKDFLDNEENEDILEDIVLTLTLFEDREMIEERLKTYAH |
| | | LFDDKVMKQLKRRRYTGWGRLSRKLINGIRDKQSGKTILDFLKSDG |
| | | FANRNFMQLIHDDSLTFKEDIQKAQVSGQGDSLHEHIANLAGSPAIK |
| | | KGILQTVKVVDELVKVMGRHKPENIVIEMARENQTTQKGQKNSRER |
| | | MKRIEEGIKELGSQILKEHPVENTQLQNEKLYLYYLQNGRDMYVDQ |
| | | ELDINRLSDYDVDAIVPQSFLKDDSIDNKVLTRSDKNRGKSDNVPSE |
| | | EVVKKMKNYWRQLLNAKLITQRKFDNLTKAERGGLSELDKAGFIKR |
| | | QLVETRQITKHVAQILDSRMNTKYDENDKLIREVKVITLKSKLVSDF |
| | | RKDFQFYKVREINNYHHAHDAYLNAVVGTALIKKYPKLESEFVYGD |
| | | YKVYDVRKMIAKSEQEIGKATAKYFFYSNIMNFFKTEITLANGEIRK |
| | | RPLIETNGETGEIVWDKGRDFATVRKVLSMPQVNIVKKTEVQTGGFS |
| | | KESILPKRNSDKLIARKKDWDPKKYGGFDSPTVAYSVLVVAKVEKG |
| | | KSKKLKSVKELLGITIMERSSFEKNPIDFLEAKGYKEVKKDLIIKLPKY |
| | | SLFELENGRKRMLASAGELQKGNELALPSKYVNFLYLASHYEKLKG |
| | | SPEDNEQKQLFVEQHKHYLDEIIEQISEFSKRVILADANLDKVLSAYN |
| | | KHRDKPIREQAENIIHLFTLTNLGAPAAFKYFDTTIDRKRYTSTKEVL |
| | | DATLIHQSITGLYETRIDLSQLGGDPKKKRKVSGSETPGTSESATPEST |
| | | GRTLVTFKDVFVDFTREEWKLLDTAQQIVYRNVMLENYKNLVSLG |
| | | YQLTKPDVILRLEKGEEPSTEPSEGSAPGTSTEPSETGMGQTGKKSEK |
| | | GPVCWRKRVKSEYMRLRQLKRFRRADEVKSMFSSNRQKILERTEIL |
| | | NQEWKQRRIQPVHILTSVSSLRGTRECSVTSDLDFPTQVIPLKTLNAV |
| | | ASVPIMYSWSPLQQNFMVEDETVLHNIPYMGDEVLDQDGTFIEELIK |
| | | NYDGKVHGDRECGFINDEIFVELVNALGQYNDDDDDDDGDDPEERE |
| | | EKQKDLEDHRDDKESRPPRKFPSDKIFEAISSMFPDKGTAEELKEKY |
| | | KELTEQQLPGALPPECTPNIDGPNAKSVQREQSLHSFHTLFCRRCFKY |
| | | DCFLHPFHATPNTYKRKNTETALDNKPCGPQCYQHLEGAKEFAAAL |
| | | TAERIKTPPKRPGGRRRGRLPNNSSRPSTPTINVLESKDTDSDREAGT |
| | | ETGGENNDKEEEEKKDETSSSSEANSRCQTPIKMKPNIEPPENVEWS |
| | | GAEASMFRVLIGTYYDNFCAIARLIGTKTCRQVYEFRVKESSIIAPAP |
| | | AEDVDTPPRKKKRKHRLWAAHCRKIQLKKDGSSNHVYNYQPCDHP |
| | | RQPCDSSCPCVIAQNFCEKFCQCSSECQNRFPGCRCKAQCNTKQCPC |
| | | YLAVRECDPDLCLTCGAADHWDSKNVSCKNCSIQRGSKKHLLLAPS |
| | | DVAGWGIFIKDPVQKNEFISEYCGEIISQDEADRRGKVYDKYMCSFL |
| | | FNLNNDFVVDATRKGNKIRFANHSVNPNCYAKVMMVNGDHRIGIF |
| | | AKRAIQTGEELFFDYRYSQADALKYVGIEREMEIP |
| 1022 | Cas-ZIM3 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMNNSQGRVTFEDVTVNFTQGEWQRLNPEQRNLYRDVMLENYSN |
| | | LVSVGQGETTKPDVILRLEQGKEPWLEEEEVLGSGRAEKNGDIGGQI |
| | | WKPKDVKESL |
| 1023 | Cas- | MGTPKKKRKVMDKKYSIGLAIGTNSVGWAVITDEYKVPSKKFKVLG |
| | ZNF554 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMFSQEERMAAGYLPRWSQELVTFEDVSMDFSQEEWELLEPAQK |
| | | NLYREVMLENYRNVVSLEALKNQCTDVGIKEGPLSPAQTSQVTSLSS |
| | | WTGYLLFQPVASSHLEQREALWIEEKGTPQASCSDWMTVLRNQDST |
| | | YKKVALQE |
| 1024 | Cas- | MGTPKKKRKVMDKKYSIGLAIGTNSVGWAVITDEYKVPSKKFKVLG |
| | ZNF264 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMAAAVLTDRAQVSVTFDDVAVTFTKEEWGQLDLAQRTLYQEV |
| | | MLENCGLLVSLGCPVPKAELICHLEHGQEPWTRKEDLSQDTCPGDK |
| | | GKPKTTEPTTCEPALSE |
| | Cas- | MGTPKKKRKVMDKKYSIGLAIGTNSVGWAVITDEYKVPSKKFKVLG |
| 1025 | ZNF354A | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMAAGQREARPQVSLTFEDVAVLFTRDEWRKLAPSQRNLYRDVM |
| | | LENYRNLVSLGLPFTKPKVISLLQQGEDPWEVEKDGSGVSSLGSKSS |
| | | HKTTKSTQTQDSSFQ |
| 1026 | Cas- | MGTPKKKRKVMDKKYSIGLAIGTNSVGWAVITDEYKVPSKKFKVLG |
| | ZNF324 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMAFEDVAVYFSQEEWGLLDTAQRALYRRVMLDNFALVASLGLS |
| | | TSRPRVVIQLERGEEPWVPSGTDTTLSRTTYRRRNPGSWSLTEDRDV |
| | | SG |
| 1027 | Cas-ZFP28 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGNKKLEAVGTGIEPKAMSQGLVTFGDVAVDFSQEEWEWLNPIQRN |
| | | LYRKVMLENYRNLASLGLCVSKPDVISSLEQGKEPW |
| 1028 | Cas-ZN627 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGDSVAFEDVAVNFTLEEWALLDPSQKNLYRDVMRETFRNLASVG |
| | | KQWEDQNIEDPFKIPRRNISHIPERLCESKEGGQGEE |
| 1029 | Cas-ZN793 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGIEYQIPVSFKDVVVGFTQEEWHRLSPAQRALYRDVMLETYSNLVS |
| | | VGYEGTKPDVILRLEQEEAPWIGEAACPGCHCWED |
| 1030 | Cas-ZN736 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGGVLTFRDVAVEFSPEEWECLDSAQQRLYRDVMLENYGNLVSLGL |
| | | AIFKPDLMTCLEQRKEPWKVKRQEAVAKHPAGSFHF |
| 1031 | Cas-ZN577 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGNATIVMSVRREQGSSSGEGSLSFEDVAVGFTREEWQFLDQSQKVL |
| | | YKEVMLENYINLVSIGYRGTKPDSLFKLEQGEPPG |
| 1032 | Cas- | MGTPKKKRKVMDKKYSIGLAIGTNSVGWAVITDEYKVPSKKFKVLG |
| | SUMO1 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGEGEYIKLKVIGQDSSEIHFKVKMTTHLKKLKESYCQRQGVPMNSL |
| | | RFLFEGQRIADNHTPKELGMEEEDVIEVYQEQTGG |
| 1033 | Cas- | MGTPKKKRKVMDKKYSIGLAIGTNSVGWAVITDEYKVPSKKFKVLG |
| | SUMO3 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGENDHINLKVAGQDGSVVQFKIKRHTPLSKLMKAYCERQGLSMRQ |
| | | IRFRFDGQPINETDTPAQLEMEDEDTIDVFQQQTGG |
| 1034 | Cas-MPP8 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGAEAFGDSEEDGEDVFEVEKILDMKTEGGKVLYKVRWKGYTSDD |
| | | DTWEPEIHLEDCKEVLLEFRKKIAENKAKAVRKDIQR |
| 1035 | Cas-RYBP | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGPSEANSIQSANATTKTSETNHTSRPRLKNVDRSTAQQLAVTVGNV |
| | | TVIITDFKEKTRSSSTSSSTVTSSAGSEQQNQSSS |
| 1036 | Cas-YAF2 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGKDKVEKEKSEKETTSKKNSHKKTRPRLKNVDRSSAQHLEVTVGD |
| | | LTVIITDFKEKTKSPPASSAASADQHSQSGSSSDNT |
| 1037 | Cas- | MGTPKKKRKVMDKKYSIGLAIGTNSVGWAVITDEYKVPSKKFKVLG |
| | SUMO5 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGKDEDIKLRVIGQDSSEIHFKVKMTTPLKKLKKSYCQRQGVPVNSL |
| | | RFLFEGQRIADNHTPEELGMEEEDVIEVYQEQIGG |
| 1038 | Cas-CBX4 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGRSEAGEPPSSLQVKPETPASAAVAVAAAAAPTTTAEKPPAEAQDE |
| | | PAESLSEFKPFFGNIIITDVTANCLTVTFKEYVTV |
| 1039 | Cas- | MGTPKKKRKVMDKKYSIGLAIGTNSVGWAVITDEYKVPSKKFKVLG |
| | PCGF2 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGHRTTRIKITELNPHLMCALCGGYFIDATTIVECLHSFCKTCIVRYLE |
| | | TNKYCPMCDVQVHKTRPLLSIRSDKTLQDIVYK |
| 1040 | Cas-CDY2 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGASQEFEVEAIVDKRQDKNGNTQYLVRWKGYDKQDDTWEPEQHL |
| | | MNCEKCVHDFNRRQTEKQKKLTWTTTSRIFSNNARRR |
| 1041 | Cas- | MGTPKKKRKVMDKKYSIGLAIGTNSVGWAVITDEYKVPSKKFKVLG |
| | CDYL2 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGASGDLYEVERIVDKRKNKKGKWEYLIRWKGYGSTEDTWEPEHH |
| | | LLHCEEFIDEFNGLHMSKDKRIKSGKQSSTSKLLRDS |
| 1042 | Cas- | MGTPKKKRKVMDKKYSIGLAIGTNSVGWAVITDEYKVPSKKFKVLG |
| | HERC2 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGTLIRKADLENHNKDGGFWTVIDGKVYDIKDFQTQSLTGNSILAQF |
| | | AGEDPVVALEAALQFEDTRESMHAFCVGQYLEPDQ |
| 1043 | Cas-ID2 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGSDHSLGISRSKTPVDDPMSLLYNMNDCYSKLKELVPSIPQNKKVS |
| | | KMEILQHVIDYILDLQIALDSHPTIVSLHHQRPGQ |
| 1044 | Cas-TOX | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGKDPNEPQKPVSAYALFFRDTQAAIKGQNPNATFGEVSKIVASMW |
| | | DGLGEEQKQVYKKKTEAAKKEYLKQLAAYRASLVSK |
| 1045 | Cas- | MGTPKKKRKVMDKKYSIGLAIGTNSVGWAVITDEYKVPSKKFKVLG |
| | SCMH1 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGDASRLSGRDPSSWTVEDVMQFVREADPQLGPHADLFRKHEIDGK |
| | | ALLLLRSDMMMKYMGLKLGPALKLSYHIDRLKQGKF |
| 1046 | Cas-CBX7 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGELSAIGEQVFAVESIRKKRVRKGKVEYLVKWKGWPPKYSTWEPE |
| | | EHILDPRLVMAYEEKEERDRASGYRKRGPKPKRLLL |
| 1047 | Cas-ID1 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGGGAGARLPALLDEQQVNVLLYDMNGCYSRLKELVPTLPQNRKV |
| | | SKVEILQHVIDYIRDLQLELNSESEVGTPGGRGLPVR |
| 1048 | Cas-CREM | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGVVMAASPGSLHSPQQLAEEATRKRELRLMKNREAAKECRRRKK |
| | | EYVKCLESRVAVLEVQNKKLIEELETLKDICSPKTDY |
| 1049 | Cas-SCX | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGGGGPGGRPGREPRQRHTANARERDRTNSVNTAFTALRTLIPTEPA |
| | | DRKLSKIETLRLASSYISHLGNVLLAGEACGDGQP |
| 1050 | Cas- | MGTPKKKRKVMDKKYSIGLAIGTNSVGWAVITDEYKVPSKKFKVLG |
| | ASCL1 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGSGFGYSLPQQQPAAVARRNERERNRVKLVNLGFATLREHVPNGA |
| | | ANKKMSKVETLRSAVEYIRALQQLLDEHDAVSAAFQ |
| 1051 | Cas- | MGTPKKKRKVMDKKYSIGLAIGTNSVGWAVITDEYKVPSKKFKVLG |
| | SCML2 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGKQGFSKDPSTWSVDEVIQFMKHTDPQISGPLADLFRQHEIDGKAL |
| | | FLLKSDVMMKYMGLKLGPALKLCYYIEKLKEGKYS |
| 1052 | Cas- | MGTPKKKRKVMDKKYSIGLAIGTNSVGWAVITDEYKVPSKKFKVLG |
| | TWST1 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGSGGGSPQSYEELQTQRVMANVRERQRTQSLNEAFAALRKIIPTLP |
| | | SDKLSKIQTLKLAARYIDFLYQVLQSDELDSKMAS |
| 1053 | Cas- | MGTPKKKRKVMDKKYSIGLAIGTNSVGWAVITDEYKVPSKKFKVLG |
| | CREB1 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGIAPGVVMASSPALPTQPAEEAARKREVRLMKNREAARECRRKKK |
| | | EYVKCLENRVAVLENQNKTLIEELKALKDLYCHKSD |
| 1054 | Cas- | MGTPKKKRKVMDKKYSIGLAIGTNSVGWAVITDEYKVPSKKFKVLG |
| | TERF1 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGSRIPVSKSQPVTPEKHRARKRQAWLWEEDKNLRSGVRKYGEGN |
| | | WSKILLHYKFNNRTSVMLKDRWRTMKKLKLISSDSED |
| 1055 | Cas-ID3 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGSLAIARGRGKGPAAEEPLSLLDDMNHCYSRLRELVPGVPRGTQLS |
| | | QVEILQRVIDYILDLQVVLAEPAPGPPDGPHLPIQ |
| 1056 | Cas-CBX8 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGGSGPPSSGGGLYRDMGAQGGRPSLIARIPVARILGDPEEESWSPSL |
| | | TNLEKVVVTDVTSNFLTVTIKESNTDQGFFKEKR |
| 1057 | Cas-CBX4 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGELPAVGEHVFAVESIEKKRIRKGRVEYLVKWRGWSPKYNTWEPE |
| | | ENILDPRLLIAFQNRERQEQLMGYRKRGPKPKPLVV |
| 1058 | Cas-GSX1 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGVDSSSNQLPSSKRMRTAFTSTQLLELEREFASNMYLSRLRRIEIAT |
| | | YLNLSEKQVKIWFQNRRVKHKKEGKGSNHRGGGG |
| 1059 | Cas- | MGTPKKKRKVMDKKYSIGLAIGTNSVGWAVITDEYKVPSKKFKVLG |
| | NKX22 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGTPGGGGDAGKKRKRRVLFSKAQTYELERRFRQQRYLSAPEREHL |
| | | ASLIRLTPTQVKIWFQNHRYKMKRARAEKGMEVTPL |
| 1060 | Cas-ATF1 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGQTVVMTSPVTLTSQTTKTDDPQLKREIRLMKNREAARECRRKKK |
| | | EYVKCLENRVAVLENQNKTLIEELKTLKDLYSNKSV |
| 1061 | Cas- | MGTPKKKRKVMDKKYSIGLAIGTNSVGWAVITDEYKVPSKKFKVLG |
| | TWST2 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGKGSPSAQSFEELQSQRILANVRERQRTQSLNEAFAALRKIIPTLPSD |
| | | KLSKIQTLKLAARYIDFLYQVLQSDEMDNKMTS |
| 1062 | Cas-TOX3 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGKDPNEPQKPVSAYALFFRDTQAAIKGQNPNATFGEVSKIVASMW |
| | | DSLGEEQKQVYKRKTEAAKKEYLKALAAYRASLVSK |
| 1063 | Cas-TOX4 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGKDPNEPQKPVSAYALFFRDTQAAIKGQNPNATFGEVSKIVASMW |
| | | DSLGEEQKQVYKRKTEAAKKEYLKALAAYKDNQECQ |
| 1064 | Cas- | MGTPKKKRKVMDKKYSIGLAIGTNSVGWAVITDEYKVPSKKFKVLG |
| | ZMYM3 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGLDGSTWDFCSEDCKSKYLLWYCKAARCHACKRQGKLLETIHWR |
| | | GQIRHFCNQQCLLRFYSQQNQPNLDTQSGPESLLNSQ |
| 1065 | Cas-I2BP1 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGASVQASRRQWCYLCDLPKMPWAMVWDFSEAVCRGCVNFEGAD |
| | | RIELLIDAARQLKRSHVLPEGRSPGPPALKHPATKDLA |
| 1066 | Cas- | MGTPKKKRKVMDKKYSIGLAIGTNSVGWAVITDEYKVPSKKFKVLG |
| | RHXF1 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMEGPQPENMQPRTRRTKFTLLQVEELESVFRHTQYPDVPTRRELA |
| | | ENLGVTEDKVRVWFKNKRARCRRHQRELMLANELR |
| 1067 | Cas-SSX2 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGPKIMPKKPAEEGNDSEEVPEASGPQNDGKELCPPGKPTTSEKIHER |
| | | SGPKRGEHAWTHRLRERKQLVIYEEISDPEEDDE |
| 1068 | Cas-I2BPL | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGSAAQVSSSRRQSCYLCDLPRMPWAMIWDFSEPVCRGCVNYEGA |
| | | DRIEFVIETARQLKRAHGCFQDGRSPGPPPPVGVKTV |
| 1069 | Cas-CBX1 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGNKKKVEEVLEEEEEEYVVEKVLDRRVVKGKVEYLLKWKGFSDE |
| | | DNTWEPEENLDCPDLIAEFLQSQKTAHETDKSEGGKR |
| 1070 | Cas-TRI68 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGLANVVEKVRLLRLHPGMGLKGDLCERHGEKLKMFCKEDVLIMC |
| | | EACSQSPEHEAHSVVPMEDVAWEYKWELHEALEHLKK |
| 1071 | Cas- | MGTPKKKRKVMDKKYSIGLAIGTNSVGWAVITDEYKVPSKKFKVLG |
| | HXA13 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGVVSHPSDASSYRRGRKKRVPYTKVQLKELEREYATNKFITKDKR |
| | | RRISATTNLSERQVTIWFQNRRVKEKKVINKLKTTS |
| 1072 | Cas-PHC3 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGENSDLLPVAQTEPSIWTVDDVWAFIHSLPGCQDIADEFRAQEIDG |
| | | QALLLLKEDHLMSAMNIKLGPALKICARINSLKES |
| 1073 | Cas-TCF24 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGAGPGGGSRSGSGRPAAANAARERSRVQTLRHAFLELQRTLPSVPP |
| | | DTKLSKLDVLLLATTYIAHLTRSLQDDAEAPADAG |
| 1074 | Cas- | MGTPKKKRKVMDKKYSIGLAIGTNSVGWAVITDEYKVPSKKFKVLG |
| | HXB13 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGQHPPDACAFRRGRKKRIPYSKGQLRELEREYAANKFITKDKRRKI |
| | | SAATSLSERQITIWFQNRRVKEKKVLAKVKNSATP |
| 1075 | Cas-HEY1 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGSMSPTTSSQILARKRRRGIIEKRRRDRINNSLSELRRLVPSAFEKQG |
| | | SAKLEKAEILQMTVDHLKMLHTAGGKGYFDAHA |
| 1076 | Cas-PHC2 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGLVGMGHHFLPSEPTKWNVEDVYEFIRSLPGCQEIAEEFRAQEIDG |
| | | QALLLLKEDHLMSAMNIKLGPALKIYARISMLKDS |
| 1077 | Cas- | MGTPKKKRKVMDKKYSIGLAIGTNSVGWAVITDEYKVPSKKFKVLG |
| | FIGLA | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGGYSSTENLQLVLERRRVANAKERERIKNLNRGFARLKALVPFLPQ |
| | | SRKPSKVDILKGATEYIQVLSDLLEGAKDSKKQDP |
| 1078 | Cas- | MGTPKKKRKVMDKKYSIGLAIGTNSVGWAVITDEYKVPSKKFKVLG |
| | SetDB1 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMSSLPGCIGLDAATATVESEEIAELQQAVVEELGISMEELRHFIDE |
| | | ELEKMDCVQQRKKQLAELETWVIQKESEVAHVDQLFDDASRAVTN |
| | | CESLVKDFYSKLGLQYRDSSSEDESSRPTEIIEIPDEDDDVLSIDSGDA |
| | | GSRTPKDQKLREAMAALRKSAQDVQKFMDAVNKKSSSQDLHKGTL |
| | | SQMSGELSKDGDLIVSMRILGKKRTKTWHKGTLIAIQTVGPGKKYK |
| | | VKFDNKGKSLLSGNHIAYDYHPPADKLYVGSRVVAKYKDGNQVWL |
| | | YAGIVAETPNVKNKLRFLIFFDDGYASYVTQSELYPICRPLKKTWEDI |
| | | EDISCRDFIEEYVTAYPNRPMVLLKSGQLIKTEWEGTWWKSRVEEV |
| | | DGSLVRILFLDDKRCEWIYRGSTRLEPMFSMKTSSASALEKKQGQLR |
| | | TRPNMGAVRSKGPVVQYTQDLTGTGTQFKPVEPPQPTAPPAPPFPPA |
| | | PPLSPQAGDSDLESQLAQSRKQVAKKSTSFRPGSVGSGHSSPTSPALS |
| | | ENVSGGKPGINQTYRSPLGSTASAPAPSALPAPPAPPVFHGMLERAPA |
| | | EPSYRAPMEKLFYLPHVCSYTCLSRVRPMRNEQYRGKNPLLVPLLY |
| | | DFRRMTARRRVNRKMGFHVIYKTPCGLCLRTMQEIERYLFETGCDF |
| | | LFLEMFCLDPYVLVDRKFQPYKPFYYILDITYGKEDVPLSCVNEIDTT |
| | | PPPQVAYSKERIPGKGVFINTGPEFLVGCDCKDGCRDKSKCACHQLT |
| | | IQATACTPGGQINPNSGYQYKRLEECLPTGVYECNKRCKCDPNMCT |
| | | NRLVQHGLQVRLQLFKTQNKGWGIRCLDDIAKGSFVCIYAGKILTD |
| | | DFADKEGLEMGDEYFANLDHIESVENFKEGYESDAPCSSDSSGVDLK |
| | | DQEDGNSGTEDPEESNDDSSDDNFCKDEDFSTSSVWRSYATRRQTR |
| | | GQKENGLSETTSKDSHPPDLGPPHIPVPPSIPVGGCNPPSSEETPKNKV |
| | | ASWLSCNSVSEGGFADSDSHSSFKTNEGGEGRAGGSRMEAEKASTS |
| | | GLGIKDEGDIKQAKKEDTDDRNKMSVVTESSRNYGYNPSPVKPEGL |
| | | RRPPSKTSMHQSRRLMASAQSNPDDVLTLSSSTESEGESGTSRKPTA |
| | | GQTSATAVDSDDIQTISSGSEGDDFEDKKNMTGPMKRQVAVKSTRG |
| | | FALKSTHGIAIKSTNMASVDKGESAPVRKNTRQFYDGEESCYIIDAKL |
| | | EGNLGRYLNHSCSPNLFVQNVFVDTHDLRFPWVAFFASKRIRAGTEL |
| | | TWDYNYEVGSVEGKELLCCCGAIECRGRLL |
| 1079 | Cas-MBD1 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMAEDWLDCPALGPGWKRREVFRKSGATCGRSDTYYQSPTGDRI |
| | | RSKVELTRYLGPACDLTLFDFKQGILCYPAPKAHPVAVASKKRKKPS |
| | | RPAKTRKRQVGPQSGEVRKEAPRDETKADTDTAPASFPAPGCCENC |
| | | GISFSGDGTQRQRLKTLCKDCRAQRIAFNREQRMFKRVGCGECAAC |
| | | QVTEDCGACSTCLLQLPHDVASGLFCKCERRRCLRIVERSRGCGVCR |
| | | GCQTQEDCGHCPICLRPPRPGLRRQWKCVQRRCLRGKHARRKGGC |
| | | DSKMAARRRPGAQPLPPPPPSQSPEPTEPHPRALAPSPPAEFIYYCVD |
| | | EDELQPYTNRRQNRKCGACAACLRRMDCGRCDFCCDKPKFGGSNQ |
| | | KRQKCRWRQCLQFAMKRLLPSVWSESEDGAGSPPPYRRRKRPSSAR |
| | | RHHLGPTLKPTLATRTAQPDHTQAPTKQEAGGGFVLPPPGTDLVFLR |
| | | EGASSPVQVPGPVAASTEALLQEAQCSGLSWVVALPQVKQEKADTQ |
| | | DEWTPGTAVLTSPVLVPGCPSKAVDPGLPSVKQEPPDPEEDKEENKD |
| | | DSASKLAPEEEAGGAGTPVITEIFSLGGTRFRDTAVWLPRSKDLKKP |
| | | GARKQ |
| 1080 | Cas-MBD2 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMRAHPGGGRCCPEQEEGESAAGGSGAGGDSAIEQGGQGSALAPS |
| | | PVSGVRREGARGGGRGRGRWKQAGRGGGVCGRGRGRGRGRGRGR |
| | | GRGRGRGRPPSGGSGLGGDGGGCGGGGSGGGGAPRREPVPFPSGSA |
| | | GPGPRGPRATESGKRMDCPALPPGWKKEEVIRKSGLSAGKSDVYYF |
| | | SPSGKKFRSKPQLARYLGNTVDLSSFDFRTGKMMPSKLQKNKQRLR |
| | | NDPLNQNKGKPDLNTTLPIRQTASIFKQPVTKVTNHPSNKVKSDPQR |
| | | MNEQPRQLFWEKRLQGLSASDVTEQIIKTMELPKGLQGVGPGSNDE |
| | | TLLSAVASALHTSSAPITGQVSAAVEKNPAVWLNTSQPLCKAFIVTD |
| | | EDIRKQEERVQQVRKKLEEALMADILSRAADTEEMDIEMDSGDEA |
| 1081 | Cas-MBD3 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMERKRWECPALPQGWEREEVPRRSGLSAGHRDVFYYSPSGKKFR |
| | | SKPQLARYLGGSMDLSTFDFRTGKMLMSKMNKSRQRVRYDSSNQV |
| | | KGKPDLNTALPVRQTASIFKQPVTKITNHPSNKVKSDPQKAVDQPRQ |
| | | LFWEKKLSGLNAFDIAEELVKTMDLPKGLQGVGPGCTDETLLSAIAS |
| | | ALHTSTMPITGQLSAAVEKNPGVWLNTTQPLCKAFMVTDEDIRKQE |
| | | ELVQQVRKRLEEALMADMLAHVEELARDGEAPLDKACAEDDDEED |
| | | EEEEEEEPDPDPEMEHV |
| 1082 | Cas-MBD4 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMGTTGLESLSLGDRGAAPTVTSSERLVPDPPNDLRKEDVAMELE |
| | | RVGEDEEQMMIKRSSECNPLLQEPIASAQFGATAGTECRKSVPCGWE |
| | | RVVKQRLFGKTAGRFDVYFISPQGLKFRSKSSLANYLHKNGETSLKP |
| | | EDFDFTVLSKRGIKSRYKDCSMAALTSHLQNQSNNSNWNLRTRSKC |
| | | KKDVFMPPSSSSELQESRGLSNFTSTHLLLKEDEGVDDVNFRKVRKP |
| | | KGKVTILKGIPIKKTKKGCRKSCSGFVQSDSKRESVCNKADAESEPV |
| | | AQKSQLDRTVCISDAGACGETLSVTSEENSLVKKKERSLSSGSNFCSE |
| | | QKTSGIINKFCSAKDSEHNEKYEDTFLESEEIGTKVEVVERKEHLHTD |
| | | ILKRGSEMDNNCSPTRKDFTGEKIFQEDTIPRTQIERRKTSLYFSSKYN |
| | | KEALSPPRRKAFKKWTPPRSPFNLVQETLFHDPWKLLIATIFLNRTSG |
| | | KMAIPVLWKFLEKYPSAEVARTADWRDVSELLKPLGLYDLRAKTIV |
| | | KFSDEYLTKQWKYPIELHGIGKYGNDSYRIFCVNEWKQVHPEDHKL |
| | | NKYHDWLWENHEKLSLS |
| 1083 | Cas- | MGTPKKKRKVMDKKYSIGLAIGTNSVGWAVITDEYKVPSKKFKVLG |
| | MeCP2 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMVAGMLGLREEKSEDQDLQGLKDKPLKFKKVKKDKKEEKEGK |
| | | HEPVQPSAHHSAEPAEAGKAETSEGSGSAPAVPEASASPKQRRSIIRD |
| | | RGPMYDDPTLPEGWTRKLKQRKSGRSAGKYDVYLINPQGKAFRSK |
| | | VELIAYFEKVGDTSLDPNDFDFTVTGRGSPSRREQKPPKKPKSPKAPG |
| | | TGRGRGRPKGSGTTRPKAATSEGVQVKRVLEKSPGKLLVKMPFQTS |
| | | PGGKAEGGGATTSTQVMVIKRPGRKRKAEADPQAIPKKRGRKPGSV |
| | | VAAAAAEAKKKAVKESSIRSVQETVLPIKKRKTRETVSIEVKEVVKP |
| | | LLVSTLGEKSGKGLKTCKSPGRKSKESSPKGRSSSASSPPKKEHHHH |
| | | HHHSESPKAPVPLLPPLPPPPPEPESSEDPTSPPEPQDLSSSVCKEEKMP |
| | | RGGSLESDGCPKEPAKTQPAVATAATAAEKYKHRGEGERKDIVSSS |
| | | MPRPNREEPVDSRTPVTERVS |
| 1084 | Cas-Kap1 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMAASAAAASAAAASAASGSPGPGEGSAGGEKRSTAPSAAASASA |
| | | SAAASSPAGGGAEALELLEHCGVCRERLRPEREPRLLPCLHSACSAC |
| | | LGPAAPAAANSSGDGGAAGDGTVVDCPVCKQQCFSKDIVENYFMR |
| | | DSGSKAATDAQDANQCCTSCEDNAPATSYCVECSEPLCETCVEAHQ |
| | | RVKYTKDHTVRSTGPAKSRDGERTVYCNVHKHEPLVLFCESCDTLT |
| | | CRDCQLNAHKDHQYQFLEDAVRNQRKLLASLVKRLGDKHATLQKS |
| | | TKEVRSSIRQVSDVQKRVQVDVKMAILQIMKELNKRGRVLVNDAQ |
| | | KVTEGQQERLERQHWTMTKIQKHQEHILRFASWALESDNNTALLLS |
| | | KKLIYFQLHRALKMIVDPVEPHGEMKFQWDLNAWTKSAEAFGKIVA |
| | | ERPGTNSTGPAPMAPPRAPGPLSKQGSGSSQPMEVQEGYGFGSGDDP |
| | | YSSAEPHVSGVKRSRSGEGEVSGLMRKVPRVSLERLDLDLTADSQPP |
| | | VFKVFPGSTTEDYNLIVIERGAAAAATGQPGTAPAGTPGAPPLAGMA |
| | | IVKEEETEAAIGAPPTATEGPETKPVLMALAEGPGAEGPRLASPSGST |
| | | SSGLEVVAPEGTSAPGGGPGTLDDSATICRVCQKPGDLVMCNQCEFC |
| | | FHLDCHLPALQDVPGEEWSCSLCHVLPDLKEEDGSLSLDGADSTGV |
| | | VAKLSPANQRKCERVLLALFCHEPCRPLHQLATDSTFSLDQPGGTLD |
| | | LTLIRARLQEKLSPPYSSPQEFAQDVGRMFKQFNKLTEDKADVQSIIG |
| | | LQRFFETRMNEAFGDTKFSAVLVEPPPMSLPGAGLSSQELSGGPGDG |
| | | P |
| 1085 | Cas-HP1a | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMGKKTKRTADSSSSEDEEEYVVEKVLDRRVVKGQVEYLLKWKG |
| | | FSEEHNTWEPEKNLDCPELISEFMKKYKKMKEGENNKPREKSESNK |
| | | RKSNFSNSADDIKSKKKREQSNDIARGFERGLEPEKIIGATDSCGDLM |
| | | FLMKWKGTDEADLVLAKEANVKCPQIVIAFYEERLTWHAYPEDAE |
| | | NKEKETAKS |
| 1086 | Cas-HP1b | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMGKKQNKKKVEEVLEEEEEEYVVEKVLDRRVVKGKVEYLLKW |
| | | KGFSDEDNTWEPEENLDCPDLIAEFLQSQKTAHETDKSEGGKRKADS |
| | | DSEDKGEESKPKKKKEESEKPRGFARGLEPERIIGATDSSGELMFLM |
| | | KWKNSDEADLVPAKEANVKCPQVVISFYEERLTWHSYPSEDDDKK |
| | | DDKN |
| 1087 | Cas-HP1g | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMASNKTTLQKMGKKQNGKSKKVEEAEPEEFVVEKVLDRRVVNG |
| | | KVEYFLKWKGFTDADNTWEPEENLDCPELIEAFLNSQKAGKEKDGT |
| | | KRKSLSDSESDDSKSKKKRDAADKPRGFARGLDPERIIGATDSSGEL |
| | | MFLMKWKDSDEADLVLAKEANMKCPQIVIAFYEERLTWHSCPEDE |
| | | AQ |
| 1088 | Cas- | MGTPKKKRKVMDKKYSIGLAIGTNSVGWAVITDEYKVPSKKFKVLG |
| | SetDB2 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMGEKNGDAKTFWMELEDDGKVDFIFEQVQNVLQSLKQKIKDGS |
| | | ATNKEYIQAMILVNEATIINSSTSIKGASQKEVNAQSSDPMPVTQKEQ |
| | | ENKSNAFPSTSCENSFPEDCTFLTTENKEILSLEDKVVDFREKDSSSNL |
| | | SYQSHDCSGACLMKMPLNLKGENPLQLPIKCHFQRRHAKTNSHSSA |
| | | LHVSYKTPCGRSLRNVEEVFRYLLETECNFLFTDNFSFNTYVQLARN |
| | | YPKQKEVVSDVDISNGVESVPISFCNEIDSRKLPQFKYRKTVWPRAY |
| | | NLTNFSSMFTDSCDCSEGCIDITKCACLQLTARNAKTSPLSSDKITTG |
| | | YKYKRLQRQIPTGIYECSLLCKCNRQLCQNRVVQHGPQVRLQVFKT |
| | | EQKGWGVRCLDDIDRGTFVCIYSGRLLSRANTEKSYGIDENGRDENT |
| | | MKNIFSKKRKLEVACSDCEVEVLPLGLETHPRTAKTEKCPPKFSNNP |
| | | KELTVETKYDNISRIQYHSVIRDPESKTAIFQHNGKKMEFVSSESVTP |
| | | EDNDGFKPPREHLNSKTKGAQKDSSSNHVDEFEDNLLIESDVIDITKY |
| | | REETPPRSRCNQATTLDNQNIKKAIEVQIQKPQEGRSTACQRQQVFC |
| | | DEELLSETKNTSSDSLTKFNKGNVFLLDATKEGNVGRFLNHSCCPNL |
| | | LVQNVFVETHNRNFPLVAFFTNRYVKARTELTWDYGYEAGTVPEKE |
| | | IFCQCGVNKCRKKIL |
| 1089 | Cas- | MGTPKKKRKVMDKKYSIGLAIGTNSVGWAVITDEYKVPSKKFKVLG |
| | SUV39H1 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMAENLKGCSVCCKSSWNQLQDLCRLAKLSCPALGISKRNLYDFE |
| | | VEYLCDYKKIREQEYYLVKWRGYPDSESTWEPRQNLKCVRILKQFH |
| | | KDLERELLRRHHRSKTPRHLDPSLANYLVQKAKQRRALRRWEQELN |
| | | AKRSHLGRITVENEVDLDGPPRAFVYINEYRVGEGITLNQVAVGCEC |
| | | QDCLWAPTGGCCPGASLHKFAYNDQGQVRLRAGLPIYECNSRCRCG |
| | | YDCPNRVVQKGIRYDLCIFRTDDGRGWGVRTLEKIRKNSFVMEYVG |
| | | EIITSEEAERRGQIYDRQGATYLFDLDYVEDVYTVDAAYYGNISHFV |
| | | NHSCDPNLQVYNVFIDNLDERLPRIAFFATRTIRAGEELTFDYNMQV |
| | | DPVDMESTRMDSNFGLAGLPGSPKKRVRIECKCGTESCRKYLF |
| 1090 | Cas- | MGTPKKKRKVMDKKYSIGLAIGTNSVGWAVITDEYKVPSKKFKVLG |
| | SUV39H1 | NTDRHSIKKNLIGALLFDSGETAEATRLKRTARRRYTRRKNRICYLQ |
| | [H320R] | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMAENLKGCSVCCKSSWNQLQDLCRLAKLSCPALGISKRNLYDFE |
| | | VEYLCDYKKIREQEYYLVKWRGYPDSESTWEPRQNLKCVRILKQFH |
| | | KDLERELLRRHHRSKTPRHLDPSLANYLVQKAKQRRALRRWEQELN |
| | | AKRSHLGRITVENEVDLDGPPRAFVYINEYRVGEGITLNQVAVGCEC |
| | | QDCLWAPTGGCCPGASLHKFAYNDQGQVRLRAGLPIYECNSRCRCG |
| | | YDCPNRVVQKGIRYDLCIFRTDDGRGWGVRTLEKIRKNSFVMEYVG |
| | | EIITSEEAERRGQIYDRQGATYLFDLDYVEDVYTVDAAYYGNISRFV |
| | | NHSCDPNLQVYNVFIDNLDERLPRIAFFATRTIRAGEELTFDYNMQV |
| | | DPVDMESTRMDSNFGLAGLPGSPKKRVRIECKCGTESCRKYLF |
| 1091 | Cas- | MGTPKKKRKVMDKKYSIGLAIGTNSVGWAVITDEYKVPSKKFKVLG |
| | SUV39H2 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMAAVGAEARGAWCVPCLVSLDTLQELCRKEKLTCKSIGITKRNL |
| | | NNYEVEYLCDYKVVKDMEYYLVKWKGWPDSTNTWEPLQNLKCPL |
| | | LLQQFSNDKHNYLSQVKKGKAITPKDNNKTLKPAIAEYIVKKAKQRI |
| | | ALQRWQDELNRRKNHKGMIFVENTVDLEGPPSDFYYINEYKPAPGIS |
| | | LVNEATFGCSCTDCFFQKCCPAEAGVLLAYNKNQQIKIPPGTPIYECN |
| | | SRCQCGPDCPNRIVQKGTQYSLCIFRTSNGRGWGVKTLVKIKRMSFV |
| | | MEYVGEVITSEEAERRGQFYDNKGITYLFDLDYESDEFTVDAARYG |
| | | NVSHFVNHSCDPNLQVFNVFIDNLDTRLPRIALFSTRTINAGEELTFD |
| | | YQMKGSGDISSDSIDHSPAKKRVRTVCKCGAVTCRGYLN |
| 1092 | Cas- | MGTPKKKRKVMDKKYSIGLAIGTNSVGWAVITDEYKVPSKKFKVLG |
| | SUV420H1 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMKWLGESKNMVVNGRRNGGKLSNDHQQNQSKLQHTGKDTLKA |
| | | GKNAVERRSNRCNGNSGFEGQSRYVPSSGMSAKELCENDDLATSLV |
| | | LDPYLGFQTHKMNTSAFPSRSSRHFSKSDSFSHNNPVRFRPIKGRQEE |
| | | LKEVIERFKKDEHLEKAFKCLTSGEWARHYFLNKNKMQEKLFKEHV |
| | | FIYLRMFATDSGFEILPCNRYSSEQNGAKIVATKEWKRNDKIELLVG |
| | | CIAELSEIEENMLLRHGENDFSVMYSTRKNCAQLWLGPAAFINHDCR |
| | | PNCKFVSTGRDTACVKALRDIEPGEEISCYYGDGFFGENNEFCECYT |
| | | CERRGTGAFKSRVGLPAPAPVINSKYGLRETDKRLNRLKKLGDSSKN |
| | | SDSQSVSSNTDADTTQEKNNATSNRKSSVGVKKNSKSRTLTRQSMS |
| | | RIPASSNSTSSKLTHINNSRVPKKLKKPAKPLLSKIKLRNHCKRLEQK |
| | | NASRKLEMGNLVLKEPKVVLYKNLPIKKDKEPEGPAQAAVASGCLT |
| | | RHAAREHRQNPVRGAHSQGESSPCTYITRRSVRTRTNLKEASDIKLE |
| | | PNTLNGYKSSVTEPCPDSGEQLQPAPVLQEEELAHETAQKGEAKCH |
| | | KSDTGMSKKKSRQGKLVKQFAKIEESTPVHDSPGKDDAVPDLMGPH |
| | | SDQGEHSGTVGVPVSYTDCAPSPVGCSVVTSDSFKTKDSFRTAKSKK |
| | | KRRITRYDAQLILENNSGIPKLTLRRRHDSSSKINDQENDGMNSSKIS |
| | | IKLSKDHDNDNNLYVAKLNNGFNSGSGSSSTKLKIQLKRDEENRGSY |
| | | TEGLHENGVCCSDPLSLLESRMEVDDYSQYEEESTDDSSSSEGDEEE |
| | | DDYDDDFEDDFIPLPPAKRLRLIVGKDSIDIDISSRRREDQSLRLNA |
| 1093 | Cas- | MGTPKKKRKVMDKKYSIGLAIGTNSVGWAVITDEYKVPSKKFKVLG |
| | SUV420H2 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMGPDRVTARELCENDDLATSLVLDPYLGFRTHKMNVSPVPPLRR |
| | | QQHLRSALETFLRQRDLEAAYRALTLGGWTARYFQSRGPRQEAALK |
| | | THVYRYLRAFLPESGFTILPCTRYSMETNGAKIVSTRAWKKNEKLEL |
| | | LVGCIAELREADEGLLRAGENDFSIMYSTRKRSAQLWLGPAAFINHD |
| | | CKPNCKFVPADGNAACVKVLRDIEPGDEVTCFYGEGFFGEKNEHCE |
| | | CHTCERKGEGAFRTRPREPALPPRPLDKYQLRETKRRLQQGLDSGSR |
| | | QGLLGPRACVHPSPLRRDPFCAACQPLRLPACSARPDTSPLWLQWLP |
| | | QPQPRVRPRKRRRPRPRRAPVLSTHHAARVSLHRWGGCGPHCRLRG |
| | | EALVALGQPPHARWAPQQDWHWARRYGLPYVVRVDLRRLAPAPP |
| | | ATPAPAGTPGPILIPKQALAFAPFSPPKRLRLVVSHGSIDLDVGGEEL |
| 1094 | Cas-EZH1 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMEIPNPPTSKCITYWKRKVKSEYMRLRQLKRLQANMGAKALYV |
| | | ANFAKVQEKTQILNEEWKKLRVQPVQSMKPVSGHPFLKKCTIESIFP |
| | | GFASQHMLMRSLNTVALVPIMYSWSPLQQNFMVEDETVLCNIPYMG |
| | | DEVKEEDETFIEELINNYDGKVHGEEEMIPGSVLISDAVFLELVDALN |
| | | QYSDEEEEGHNDTSDGKQDDSKEDLPVTRKRKRHAIEGNKKSSKKQ |
| | | FPNDMIFSAIASMFPENGVPDDMKERYRELTEMSDPNALPPQCTPNI |
| | | DGPNAKSVQREQSLHSFHTLFCRRCFKYDCFLHPFHATPNVYKRKN |
| | | KEIKIEPEPCGTDCFLLLEGAKEYAMLHNPRSKCSGRRRRRHHIVSAS |
| | | CSNASASAVAETKEGDSDRDTGNDWASSSSEANSRCQTPTKQKASP |
| | | APPQLCVVEAPSEPVEWTGAEESLFRVFHGTYFNNFCSIARLLGTKT |
| | | CKQVFQFAVKESLILKLPTDELMNPSQKKKRKHRLWAAHCRKIQLK |
| | | KDNSSTQVYNYQPCDHPDRPCDSTCPCIMTQNFCEKFCQCNPDCQN |
| | | RFPGCRCKTQCNTKQCPCYLAVRECDPDLCLTCGASEHWDCKVVSC |
| | | KNCSIQRGLKKHLLLAPSDVAGWGTFIKESVQKNEFISEYCGELISQD |
| | | EADRRGKVYDKYMSSFLFNLNNDFVVDATRKGNKIRFANHSVNPN |
| | | CYAKVVMVNGDHRIGIFAKRAIQAGEELFFDYRYSQADALKYVGIE |
| | | RETDVL |
| 1095 | Cas-EZH2 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMGQTGKKSEKGPVCWRKRVKSEYMRLRQLKRFRRADEVKSMFS |
| | | SNRQKILERTEILNQEWKQRRIQPVHILTSVSSLRGTRECSVTSDLDFP |
| | | TQVIPLKTLNAVASVPIMYSWSPLQQNFMVEDETVLHNIPYMGDEV |
| | | LDQDGTFIEELIKNYDGKVHGDRECGFINDEIFVELVNALGQYNDDD |
| | | DDDDGDDPEEREEKQKDLEDHRDDKESRPPRKFPSDKIFEAISSMFP |
| | | DKGTAEELKEKYKELTEQQLPGALPPECTPNIDGPNAKSVQREQSLH |
| | | SFHTLFCRRCFKYDCFLHPFHATPNTYKRKNTETALDNKPCGPQCYQ |
| | | HLEGAKEFAAALTAERIKTPPKRPGGRRRGRLPNNSSRPSTPTINVLE |
| | | SKDTDSDREAGTETGGENNDKEEEEKKDETSSSSEANSRCQTPIKMK |
| | | PNIEPPENVEWSGAEASMFRVLIGTYYDNFCAIARLIGTKTCRQVYEF |
| | | RVKESSIIAPAPAEDVDTPPRKKKRKHRLWAAHCRKIQLKKDGSSNH |
| | | VYNYQPCDHPRQPCDSSCPCVIAQNFCEKFCQCSSECQNRFPGCRCK |
| | | AQCNTKQCPCYLAVRECDPDLCLTCGAADHWDSKNVSCKNCSIQR |
| | | GSKKHLLLAPSDVAGWGIFIKDPVQKNEFISEYCGEIISQDEADRRGK |
| | | VYDKYMCSFLFNLNNDFVVDATRKGNKIRFANHSVNPNCYAKVMM |
| | | VNGDHRIGIFAKRAIQTGEELFFDYRYSQADALKYVGIEREMEIP |
| 1096 | Cas- | MGTPKKKRKVMDKKYSIGLAIGTNSVGWAVITDEYKVPSKKFKVLG |
| | EZH2 | NTDRHSIKKNLIGALLFDSGETAEATRLKRTARRRYTRRKNRICYLQ |
| | [S21A] | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMGQTGKKSEKGPVCWRKRVKAEYMRLRQLKRFRRADEVKSMF |
| | | SSNRQKILERTEILNQEWKQRRIQPVHILTSVSSLRGTRECSVTSDLDF |
| | | PTQVIPLKTLNAVASVPIMYSWSPLQQNFMVEDETVLHNIPYMGDEV |
| | | LDQDGTFIEELIKNYDGKVHGDRECGFINDEIFVELVNALGQYNDDD |
| | | DDDDGDDPEEREEKQKDLEDHRDDKESRPPRKFPSDKIFEAISSMFP |
| | | DKGTAEELKEKYKELTEQQLPGALPPECTPNIDGPNAKSVQREQSLH |
| | | SFHTLFCRRCFKYDCFLHPFHATPNTYKRKNTETALDNKPCGPQCYQ |
| | | HLEGAKEFAAALTAERIKTPPKRPGGRRRGRLPNNSSRPSTPTINVLE |
| | | SKDTDSDREAGTETGGENNDKEEEEKKDETSSSSEANSRCQTPIKMK |
| | | PNIEPPENVEWSGAEASMFRVLIGTYYDNFCAIARLIGTKTCRQVYEF |
| | | RVKESSIIAPAPAEDVDTPPRKKKRKHRLWAAHCRKIQLKKDGSSNH |
| | | VYNYQPCDHPRQPCDSSCPCVIAQNFCEKFCQCSSECQNRFPGCRCK |
| | | AQCNTKQCPCYLAVRECDPDLCLTCGAADHWDSKNVSCKNCSIQR |
| | | GSKKHLLLAPSDVAGWGIFIKDPVQKNEFISEYCGEIISQDEADRRGK |
| | | VYDKYMCSFLFNLNNDFVVDATRKGNKIRFANHSVNPNCYAKVMM |
| | | VNGDHRIGIFAKRAIQTGEELFFDYRYSQADALKYVGIEREMEIP |
| 1097 | Cas- | MGTPKKKRKVMDKKYSIGLAIGTNSVGWAVITDEYKVPSKKFKVLG |
| | EHMT1 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMAAADAEAVPARGEPQQDCCVKTELLGEETPMAADEGSAEKQA |
| | | GEAHMAADGETNGSCENSDASSHANAAKHTQDSARVNPQDGTNTL |
| | | TRIAENGVSERDSEAAKQNHVTADDFVQTSVIGSNGYILNKPALQAQ |
| | | PLRTTSTLASSLPGHAAKTLPGGAGKGRTPSAFPQTPAAPPATLGEGS |
| | | ADTEDRKLPAPGADVKVHRARKTMPKSVVGLHAASKDPREVREAR |
| | | DHKEPKEEINKNISDFGRQQLLPPFPSLHQSLPQNQCYMATTKSQTA |
| | | CLPFVLAAAVSRKKKRRMGTYSLVPKKKTKVLKQRTVIEMFKSITHS |
| | | TVGSKGEKDLGASSLHVNGESLEMDSDEDDSEELEEDDGHGAEQAA |
| | | AFPTEDSRTSKESMSEADRAQKMDGESEEEQESVDTGEEEEGGDESD |
| | | LSSESSIKKKFLKRKGKTDSPWIKPARKRRRRSRKKPSGALGSESYKS |
| | | SAGSAEQTAPGDSTGYMEVSLDSLDLRVKGILSSQAEGLANGPDVLE |
| | | TDGLQEVPLCSCRMETPKSREITTLANNQCMATESVDHELGRCTNSV |
| | | VKYELMRPSNKAPLLVLCEDHRGRMVKHQCCPGCGYFCTAGNFME |
| | | CQPESSISHRFHKDCASRVNNASYCPHCGEESSKAKEVTIAKADTTST |
| | | VTPVPGQEKGSALEGRADTTTGSAAGPPLSEDDKLQGAASHVPEGF |
| | | DPTGPAGLGRPTPGLSQGPGKETLESALIALDSEKPKKLRFHPKQLYF |
| | | SARQGELQKVLLMLVDGIDPNFKMEHQNKRSPLHAAAEAGHVDICH |
| | | MLVQAGANIDTCSEDQRTPLMEAAENNHLEAVKYLIKAGALVDPK |
| | | DAEGSTCLHLAAKKGHYEVVQYLLSNGQMDVNCQDDGGWTPMIW |
| | | ATEYKHVDLVKLLLSKGSDINIRDNEENICLHWAAFSGCVDIAEILLA |
| | | AKCDLHAVNIHGDSPLHIAARENRYDCVVLFLSRDSDVTLKNKEGE |
| | | TPLQCASLNSQVWSALQMSKALQDSAPDRPSPVERIVSRDIARGYER |
| | | IPIPCVNAVDSEPCPSNYKYVSQNCVTSPMNIDRNITHLQYCVCIDDC |
| | | SSSNCMCGQLSMRCWYDKDGRLLPEFNMAEPPLIFECNHACSCWRN |
| | | CRNRVVQNGLRARLQLYRTRDMGWGVRSLQDIPPGTFVCEYVGELI |
| | | SDSEADVREEDSYLFDLDNKDGEVYCIDARFYGNVSRFINHHCEPNL |
| | | VPVRVFMAHQDLRFPRIAFFSTRLIEAGEQLGFDYGERFWDIKGKLFS |
| | | CRCGSPKCRHSSAALAQRQASAAQEAQEDGLPDTSSAAAADPL |
| 1098 | Cas- | MGTPKKKRKVMDKKYSIGLAIGTNSVGWAVITDEYKVPSKKFKVLG |
| | EHMT2 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMAAAAGAAAAAAAEGEAPAEMGALLLEKETRGATERVHGSLG |
| | | DTPRSEETLPKATPDSLEPAGPSSPASVTVTVGDEGADTPVGATPLIG |
| | | DESENLEGDGDLRGGRILLGHATKSFPSSPSKGGSCPSRAKMSMTGA |
| | | GKSPPSVQSLAMRLLSMPGAQGAAAAGSEPPPATTSPEGQPKVHRA |
| | | RKTMSKPGNGQPPVPEKRPPEIQHFRMSDDVHSLGKVTSDLAKRRK |
| | | LNSGGGLSEELGSARRSGEVTLTKGDPGSLEEWETVVGDDFSLYYDS |
| | | YSVDERVDSDSKSEVEALTEQLSEEEEEEEEEEEEEEEEEEEEEEEED |
| | | EESGNQSDRSGSSGRRKAKKKWRKDSPWVKPSRKRRKREPPRAKEP |
| | | RGVNGVGSSGPSEYMEVPLGSLELPSEGTLSPNHAGVSNDTSSLETE |
| | | RGFEELPLCSCRMEAPKIDRISERAGHKCMATESVDGELSGCNAAIL |
| | | KRETMRPSSRVALMVLCETHRARMVKHHCCPGCGYFCTAGTFLEC |
| | | HPDFRVAHRFHKACVSQLNGMVFCPHCGEDASEAQEVTIPRGDGVT |
| | | PPAGTAAPAPPPLSQDVPGRADTSQPSARMRGHGEPRRPPCDPLADT |
| | | IDSSGPSLTLPNGGCLSAVGLPLGPGREALEKALVIQESERRKKLRFH |
| | | PRQLYLSVKQGELQKVILMLLDNLDPNFQSDQQSKRTPLHAAAQKG |
| | | SVEICHVLLQAGANINAVDKQQRTPLMEAVVNNHLEVARYMVQRG |
| | | GCVYSKEEDGSTCLHHAAKIGNLEMVSLLLSTGQVDVNAQDSGGW |
| | | TPIIWAAEHKHIEVIRMLLTRGADVTLTDNEENICLHWASFTGSAAIA |
| | | EVLLNARCDLHAVNYHGDTPLHIAARESYHDCVLLFLSRGANPELR |
| | | NKEGDTAWDLTPERSDVWFALQLNRKLRLGVGNRAIRTEKIICRDV |
| | | ARGYENVPIPCVNGVDGEPCPEDYKYISENCETSTMNIDRNITHLQH |
| | | CTCVDDCSSSNCLCGQLSIRCWYDKDGRLLQEFNKIEPPLIFECNQAC |
| | | SCWRNCKNRVVQSGIKVRLQLYRTAKMGWGVRALQTIPQGTFICEY |
| | | VGELISDAEADVREDDSYLFDLDNKDGEVYCIDARYYGNISRFINHL |
| | | CDPNIIPVRVFMLHQDLRFPRIAFFSSRDIRTGEELGFDYGDRFWDIKS |
| | | KYFTCQCGSEKCKHSAEAIALEQSRLARLDPHPELLPELGSLPPVNT |
| 1099 | Cas-LSD1 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMLSGKKAAAAAAAAAAAATGTEAGPGTAGGSENGSEVAAQPA |
| | | GLSGPAEVGPGAVGERTPRKKEPPRASPPGGLAEPPGSAGPQAGPTV |
| | | VPGSATPMETGIAETPEGRRTSRRKRAKVEYREMDESLANLSEDEYY |
| | | SEEERNAKAEKEKKLPPPPPQAPPEEENESEPEEPSGVEGAAFQSRLP |
| | | HDRMTSQEAACFPDIISGPQQTQKVFLFIRNRTLQLWLDNPKIQLTFE |
| | | ATLQQLEAPYNSDTVLVHRVHSYLERHGLINFGIYKRIKPLPTKKTG |
| | | KVIIIGSGVSGLAAARQLQSFGMDVTLLEARDRVGGRVATFRKGNY |
| | | VADLGAMVVTGLGGNPMAVVSKQVNMELAKIKQKCPLYEANGQA |
| | | VPKEKDEMVEQEFNRLLEATSYLSHQLDFNVLNNKPVSLGQALEVV |
| | | IQLQEKHVKDEQIEHWKKIVKTQEELKELLNKMVNLKEKIKELHQQ |
| | | YKEASEVKPPRDITAEFLVKSKHRDLTALCKEYDELAETQGKLEEKL |
| | | QELEANPPSDVYLSSRDRQILDWHFANLEFANATPLSTLSLKHWDQD |
| | | DDFEFTGSHLTVRNGYSCVPVALAEGLDIKLNTAVRQVRYTASGCE |
| | | VIAVNTRSTSQTFIYKCDAVLCTLPLGVLKQQPPAVQFVPPLPEWKTS |
| | | AVQRMGFGNLNKVVLCFDRVFWDPSVNLFGHVGSTTASRGELFLF |
| | | WNLYKAPILLALVAGEAAGIMENISDDVIVGRCLAILKGIFGSSAVPQ |
| | | PKETVVSRWRADPWARGSYSYVAAGSSGNDYDLMAQPITPGPSIPG |
| | | APQPIPRLFFAGEHTIRNYPATVHGALLSGLREAGRIADQFLGAMYTL |
| | | PRQATPGVPAQQSPSM |
| 1100 | Cas-SUZ12 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMAPQKHGGGGGGGSGPSAGSGGGGFGGSAAVAAATASGGKSG |
| | | GGSCGGGGSYSASSSSSAAAAAGAAVLPVKKPKMEHVQADHELFL |
| | | QAFEKPTQIYRFLRTRNLIAPIFLHRTLTYMSHRNSRTNIKRKTFKVD |
| | | DMLSKVEKMKGEQESHSLSAHLQLTFTGFFHKNDKPSPNSENEQNS |
| | | VTLEVLLVKVCHKKRKDVSCPIRQVPTGKKQVPLNPDLNQTKPGNF |
| | | PSLAVSSNEFEPSNSHMVKSYSLLFRVTRPGRREFNGMINGETNENID |
| | | VNEELPARRKRNREDGEKTFVAQMTVFDKNRRLQLLDGEYEVAMQ |
| | | EMEECPISKKRATWETILDGKRLPPFETFSQGPTLQFTLRWTGETNDK |
| | | STAPIAKPLATRNSESLHQENKPGSVKPTQTIAVKESLTTDLQTRKEK |
| | | DTPNENRQKLRIFYQFLYNNNTRQQTEARDDLHCPWCTLNCRKLYS |
| | | LLKHLKLCHSRFIFNYVYHPKGARIDVSINECYDGSYAGNPQDIHRQ |
| | | PGFAFSRNGPVKRTPITHILVCRPKRTKASMSEFLESEDGEVEQQRTY |
| | | SSGHNRLYFHSDTCLPLRPQEMEVDSEDEKDPEWLREKTITQIEEFSD |
| | | VNEGEKEVMKLWNLHVMKHGFIADNQMNHACMLFVENYGQKIIK |
| | | KNLCRNFMLHLVSMHDFNLISIMSIDKAVTKLREMQQKLEKGESASP |
| | | ANEEITEEQNGTANGFSEINSKEKALETDSVSGVSKQSKKQKL |
| 1101 | Cas-EED | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMSEREVSTAPAGTDMPAAKKQKLSSDENSNPDLSGDENDDAVSI |
| | | ESGTNTERPDTPTNTPNAPGRKSWGKGKWKSKKCKYSFKCVNSLKE |
| | | DHNQPLFGVQFNWHSKEGDPLVFATVGSNRVTLYECHSQGEIRLLQ |
| | | SYVDADADENFYTCAWTYDSNTSHPLLAVAGSRGIIRIINPITMQCIK |
| | | HYVGHGNAINELKFHPRDPNLLLSVSKDHALRLWNIQTDTLVAIFGG |
| | | VEGHRDEVLSADYDLLGEKIMSCGMDHSLKLWRINSKRMMNAIKES |
| | | YDYNPNKTNRPFISQKIHFPDFSTRDIHRNYVDCVRWLGDLILSKSCE |
| | | NAIVCWKPGKMEDDIDKIKPSESNVTILGRFDYSQCDIWYMRFSMDF |
| | | WQKMLALGNQVGKLYVWDLEVEDPHKAKCTTLTHHKCGAAIRQT |
| | | SFSRDSSILIAVCDDASIWRWDRLR |
| 1102 | Cas- | MGTPKKKRKVMDKKYSIGLAIGTNSVGWAVITDEYKVPSKKFKVLG |
| | RING1 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMTTPANAQNASKTWELSLYELHRTPQEAIMDGTEIAVSPRSLHSE |
| | | LMCPICLDMLKNTMTTKECLHRFCSDCIVTALRSGNKECPTCRKKLV |
| | | SKRSLRPDPNFDALISKIYPSREEYEAHQDRVLIRLSRLHNQQALSSSI |
| | | EEGLRMQAMHRAQRVRRPIPGSDQTTTMSGGEGEPGEGEGDGEDVS |
| | | SDSAPDSAPGPAPKRPRGGGAGGSSVGTGGGGTGGVGGGAGSEDSG |
| | | DRGGTLGGGTLGPPSPPGAPSPPEPGGEIELVFRPHPLLVEKGEYCQT |
| | | RYVKTTGNATVDHLSKYLALRIALERRQQQEAGEPGGPGGGASDTG |
| | | GPDGCGGEGGGAGGGDGPEEPALPSLEGVSEKQYTIYIAPGGGAFTT |
| | | LNGSLTLELVNEKFWKVSRPLELCYAPTKDPK |
| 1103 | Cas- | MGTPKKKRKVMDKKYSIGLAIGTNSVGWAVITDEYKVPSKKFKVLG |
| | RING2 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMSQAVQTNGTQPLSKTWELSLYELQRTPQEAITDGLEIVVSPRSL |
| | | HSELMCPICLDMLKNTMTTKECLHRFCADCIITALRSGNKECPTCRK |
| | | KLVSKRSLRPDPNFDALISKIYPSRDEYEAHQERVLARINKHNNQQA |
| | | LSHSIEEGLKIQAMNRLQRGKKQQIENGSGAEDNGDSSHCSNASTHS |
| | | NQEAGPSNKRTKTSDDSGLELDNNNAAMAIDPVMDGASEIELVFRP |
| | | HPTLMEKDDSAQTRYIKTSGNATVDHLSKYLAVRLALEELRSKGES |
| | | NQMNLDTASEKQYTIYIATASGQFTVLNGSFSLELVSEKYWKVNKP |
| | | MELYYAPTKEHK |
| 1104 | Cas-PHC1 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMETESEQNSNSTNGSSSSGGSSRPQIAQMSLYERQAVQALQALQR |
| | | QPNAAQYFHQFMLQQQLSNAQLHSLAAVQQATIAASRQASSPNTST |
| | | TQQQTTTTQASINLATTSAAQLISRSQSVSSPSATTLTQSVLLGNTTSP |
| | | PLNQSQAQMYLRPQLGNLLQVNRTLGRNVPLASQLILMPNGAVAAV |
| | | QQEVPSAQSPGVHADADQVQNLAVRNQQASAQGPQMQGSTQKAIP |
| | | PGASPVSSLSQASSQALAVAQASSGATNQSLNLSQAGGGSGNSIPGS |
| | | MGPGGGGQAHGGLGQLPSSGMGGGSCPRKGTGVVQPLPAAQTVTV |
| | | SQGSQTEAESAAAKKAEADGSGQQNVGMNLTRTATPAPSQTLISSA |
| | | TYTQIQPHSLIQQQQQIHLQQKQVVIQQQIAIHHQQQFQHRQSQLLHT |
| | | ATHLQLAQQQQQQQQQQQQQQQPQATTLTAPQPPQVPPTQQVPPSQ |
| | | SQQQAQTLVVQPMLQSSPLSLPPDAAPKPPIPIQSKPPVAPIKPPQLGA |
| | | AKMSAAQQPPPHIPVQVVGTRQPGTAQAQALGLAQLAAAVPTSRG |
| | | MPGTVQSGQAHLASSPPSSQAPGALQECPPTLAPGMTLAPVQGTAH |
| | | VVKGGATTSSPVVAQVPAAFYMQSVHLPGKPQTLAVKRKADSEEER |
| | | DDVSTLGSMLPAKASPVAESPKVMDEKSSLGEKAESVANVNANTPS |
| | | SELVALTPAPSVPPPTLAMVSRQMGDSKPPQAIVKPQILTHIIEGFVIQ |
| | | EGAEPFPVGCSQLLKESEKPLQTGLPTGLTENQSGGPLGVDSPSAELD |
| | | KKANLLKCEYCGKYAPAEQFRGSKRFCSMTCAKRYNVSCSHQFRLK |
| | | RKKMKEFQEANYARVRRRGPRRSSSDIARAKIQGKCHRGQEDSSRG |
| | | SDNSSYDEALSPTSPGPLSVRAGHGERDLGNPNTAPPTPELHGINPVF |
| | | LSSNPSRWSVEEVYEFIASLQGCQEIAEEFRSQEIDGQALLLLKEEHL |
| | | MSAMNIKLGPALKICAKINVLKET |
| 1105 | Cas-BMI1 | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMHRTTRIKITELNPHLMCVLCGGYFIDATTIIECLHSFCKTCIVRYL |
| | | ETSKYCPICDVQVHKTRPLLNIRSDKTLQDIVYKLVPGLFKNEMKRR |
| | | RDFYAAHPSADAANGSNEDRGEVADEDKRIITDDEIISLSIEFFDQNR |
| | | LDRKVNKDKEKSKEEVNDKRYLRCPAAMTVMHLRKFLRSKMDIPN |
| | | TFQIDVMYEEEPLKDYYTLMDIAYIYTWRRNGPLPLKYRVRPTCKR |
| | | MKISHQRDGLTNAGELESDSGSDKANSPAGGIPSTSSCLPSPSTPVQS |
| | | PHPQFPHISSTMNGTSNSPSGNHQSSFANRPRKSSVNGSSATSSG |
| 1106 | Cas- | MGTPKKKRKVMDKKYSIGLAIGTNSVGWAVITDEYKVPSKKFKVLG |
| | RBBP4 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMADKEAAFDDAVEERVINEEYKIWKKNTPFLYDLVMTHALEWP |
| | | SLTAQWLPDVTRPEGKDFSIHRLVLGTHTSDEQNHLVIASVQLPNDD |
| | | AQFDASHYDSEKGEFGGFGSVSGKIEIEIKINHEGEVNRARYMPQNP |
| | | CIIATKTPSSDVLVFDYTKHPSKPDPSGECNPDLRLRGHQKEGYGLS |
| | | WNPNLSGHLLSASDDHTICLWDISAVPKEGKVVDAKTIFTGHTAVVE |
| | | DVSWHLLHESLFGSVADDQKLMIWDTRSNNTSKPSHSVDAHTAEVN |
| | | CLSFNPYSEFILATGSADKTVALWDLRNLKLKLHSFESHKDEIFQVQ |
| | | WSPHNETILASSGTDRRLNVWDLSKIGEEQSPEDAEDGPPELLFIHGG |
| | | HTAKISDFSWNPNEPWVICSVSEDNIMQVWQMAENIYNDEDPEGSV |
| | | DPEGQGS |
| 1107 | Cas- | MGTPKKKRKVMDKKYSIGLAIGTNSVGWAVITDEYKVPSKKFKVLG |
| | RBBP7 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMASKEMFEDTVEERVINEEYKIWKKNTPFLYDLVMTHALQWPSL |
| | | TVQWLPEVTKPEGKDYALHWLVLGTHTSDEQNHLVVARVHIPNDD |
| | | AQFDASHCDSDKGEFGGFGSVTGKIECEIKINHEGEVNRARYMPQNP |
| | | HIIATKTPSSDVLVFDYTKHPAKPDPSGECNPDLRLRGHQKEGYGLS |
| | | WNSNLSGHLLSASDDHTVCLWDINAGPKEGKIVDAKAIFTGHSAVV |
| | | EDVAWHLLHESLFGSVADDQKLMIWDTRSNTTSKPSHLVDAHTAEV |
| | | NCLSFNPYSEFILATGSADKTVALWDLRNLKLKLHTFESHKDEIFQV |
| | | HWSPHNETILASSGTDRRLNVWDLSKIGEEQSAEDAEDGPPELLFIHG |
| | | GHTAKISDFSWNPNEPWVICSVSEDNIMQIWQMAENIYNDEESDVTT |
| | | SELEGQGS |
| 1108 | Cas-REST | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMATQVMGQSSGGGGLFTSSGNIGMALPNDMYDLHDLSKAELAA |
| | | PQLIMLANVALTGEVNGSCCDYLVGEERQMAELMPVGDNNFSDSEE |
| | | GEGLEESADIKGEPHGLENMELRSLELSVVEPQPVFEASGAPDIYSSN |
| | | KDLPPETPGAEDKGKSSKTKPFRCKPCQYEAESEEQFVHHIRVHSAK |
| | | KFFVEESAEKQAKARESGSSTAEEGDFSKGPIRCDRCGYNTNRYDHY |
| | | TAHLKHHTRAGDNERVYKCIICTYTTVSEYHWRKHLRNHFPRKVYT |
| | | CGKCNYFSDRKNNYVQHVRTHTGERPYKCELCPYSSSQKTHLTRHM |
| | | RTHSGEKPFKCDQCSYVASNQHEVTRHARQVHNGPKPLNCPHCDY |
| | | KTADRSNFKKHVELHVNPRQFNCPVCDYAASKKCNLQYHFKSKHPT |
| | | CPNKTMDVSKVKLKKTKKREADLPDNITNEKTEIEQTKIKGDVAGK |
| | | KNEKSVKAEKRDVSKEKKPSNNVSVIQVTTRTRKSVTEVKEMDVHT |
| | | GSNSEKFSKTKKSKRKLEVDSHSLHGPVNDEESSTKKKKKVESKSKN |
| | | NSQEVPKGDSKVEENKKQNTCMKKSTKKKTLKNKSSKKSSKPPQKE |
| | | PVEKGSAQMDPPQMGPAPTEAVQKGPVQVEPPPPMEHAQMEGAQI |
| | | RPAPDEPVQMEVVQEGPAQKELLPPVEPAQMVGAQIVLAHMELPPP |
| | | METAQTEVAQMGPAPMEPAQMEVAQVESAPMQVVQKEPVQMELS |
| | | PPMEVVQKEPVQIELSPPMEVVQKEPVKIELSPPIEVVQKEPVQMELS |
| | | PPMGVVQKEPAQREPPPPREPPLHMEPISKKPPLRKDKKEKSNMQSE |
| | | RARKEQVLIEVGLVPVKDSWLLKESVSTEDLSPPSPPLPKENLREEAS |
| | | GDQKLLNTGEGNKEAPLQKVGAEEADESLPGLAANINESTHISSSGQ |
| | | NLNTPEGETLNGKHQTDSIVCEMKMDTDQNTRENLTGINSTVEEPVS |
| | | PMLPPSAVEEREAVSKTALASPPATMAANESQEIDEDEGIHSHEGSDL |
| | | SDNMSEGSDDSGLHGARPVPQESSRKNAKEALAVKAAKGDFVCIFC |
| | | DRSFRKGKDYSKHLNRHLVNVYYLEEAAQGQE |
| 1109 | Cas- | MGTPKKKRKVMDKKYSIGLAIGTNSVGWAVITDEYKVPSKKFKVLG |
| | RCOR1 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMPAMVEKGPEVSGKRRGRNNAAASASAAAASAAASAACASPAA |
| | | TAASGAAASSASAAAASAAAAPNNGQNKSLAAAAPNGNSSSNSWE |
| | | EGSSGSSSDEEHGGGGMRVGPQYQAVVPDFDPAKLARRSQERDNLG |
| | | MLVWSPNQNLSEAKLDEYIAIAKEKHGYNMEQALGMLFWHKHNIE |
| | | KSLADLPNFTPFPDEWTVEDKVLFEQAFSFHGKTFHRIQQMLPDKSI |
| | | ASLVKFYYSWKKTRTKTSVMDRHARKQKREREESEDELEEANGNN |
| | | PIDIEVDQNKESKKEVPPTETVPQVKKEKHSTQAKNRAKRKPPKGMF |
| | | LSQEDVEAVSANATAATTVLRQLDMELVSVKRQIQNIKQTNSALKE |
| | | KLDGGIEPYRLPEVIQKCNARWTTEEQLLAVQAIRKYGRDFQAISDVI |
| | | GNKSVVQVKNFFVNYRRRFNIDEVLQEWEAEHGKEETNGPSNQKPV |
| | | KSPDNSIKMPEEEDEAPVLDVRYASAS |
| 1110 | Cas-SIN3A | MGTPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMKRRLDDQESPVYAAQQRRIPGSTEAFPHQHRVLAPAPPVYEAV |
| | | SETMQSATGIQYSVTPSYQVSAMPQSSGSHGPAIAAVHSSHHHPTAV |
| | | QPHGGQVVQSHAHPAPPVAPVQGQQQFQRLKVEDALSYLDQVKLQ |
| | | FGSQPQVYNDFLDIMKEFKSQSIDTPGVISRVSQLFKGHPDLIMGFNT |
| | | FLPPGYKIEVQTNDMVNVTTPGQVHQIPTHGIQPQPQPPPQHPSQPSA |
| | | QSAPAPAQPAPQPPPAKVSKPSQLQAHTPASQQTPPLPPYASPRSPPV |
| | | QPHTPVTISLGTAPSLQNNQPVEFNHAINYVNKIKNRFQGQPDIYKAF |
| | | LEILHTYQKEQRNAKEAGGNYTPALTEQEVYAQVARLFKNQEDLLS |
| | | EFGQFLPDANSSVLLSKTTAEKVDSVRNDHGGTVKKPQLNNKPQRP |
| | | SQNGCQIRRHPTGTTPPVKKKPKLLNLKDSSMADASKHGGGTESLFF |
| | | DKVRKALRSAEAYENFLRCLVIFNQEVISRAELVQLVSPFLGKFPELF |
| | | NWFKNFLGYKESVHLETYPKERATEGIAMEIDYASCKRLGSSYRALP |
| | | KSYQQPKCTGRTPLCKEVLNDTWVSFPSWSEDSTFVSSKKTQYEEHI |
| | | YRCEDERFELDVVLETNLATIRVLEAIQKKLSRLSAEEQAKFRLDNTL |
| | | GGTSEVIHRKALQRIYADKAADIIDGLRKNPSIAVPIVLKRLKMKEEE |
| | | WREAQRGFNKVWREQNEKYYLKSLDHQGINFKQNDTKVLRSKSLL |
| | | NEIESIYDERQEQATEENAGVPVGPHLSLAYEDKQILEDAAALIIHHV |
| | | KRQTGIQKEDKYKIKQIMHHFIPDLLFAQRGDLSDVEEEEEEEMDVD |
| | | EATGAVKKHNGVGGSPPKSKLLFSNTAAQKLRGMDEVYNLFYVNN |
| | | NWYIFMRLHQILCLRLLRICSQAERQIEEENREREWEREVLGIKRDKS |
| | | DSPAIQLRLKEPMDVDVEDYYPAFLDMVRSLLDGNIDSSQYEDSLRE |
| | | MFTIHAYIAFTMDKLIQSIVRQLQHIVSDEICVQVTDLYLAENNNGAT |
| | | GGQLNTQNSRSLLESTYQRKAEQLMSDENCFKLMFIQSQGQVQLTIE |
| | | LLDTEEENSDDPVEAERWSDYVERYMNSDTTSPELREHLAQKPVFLP |
| | | RNLRRIRKCQRGREQQEKEGKEGNSKKTMENVDSLDKLECRFKLNS |
| | | YKMVYVIKSEDYMYRRTALLRAHQSHERVSKRLHQRFQAWVDKW |
| | | TKEHVPREMAAETSKWLMGEGLEGLVPCTTTCDTETLHFVSINKYR |
| | | VKYGTVFKAP |
| 1111 | Cas- | MGTPKKKRKVMDKKYSIGLAIGTNSVGWAVITDEYKVPSKKFKVLG |
| | HDAC5 | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKVGGPSSGAPPPSGGSPAGSPTSTEEGTSESA |
| | | TPESGPGTSTEPSEGSAPGSPAGSPTSTEEGTSTEPSEGSAPGTSTEPSE |
| | | TGMNSPNESDGMSGREPSLEILPRTSLHSIPVTVEVKPVLPRAMPSSM |
| | | GGGGGGSPSPVELRGALVGSVDPTLREQQLQQELLALKQQQQLQKQ |
| | | LLFAEFQKQHDHLTRQHEVQLQKHLKQQQEMLAAKQQQEMLAAK |
| | | RQQELEQQRQREQQRQEELEKQRLEQQLLILRNKEKSKESAIASTEV |
| | | KLRLQEFLLSKSKEPTPGGLNHSLPQHPKCWGAHHASLDQSSPPQSG |
| | | PPGTPPSYKLPLPGPYDSRDDFPLRKTASEPNLKVRSRLKQKVAERRS |
| | | SPLLRRKDGTVISTFKKRAVEITGAGPGASSVCNSAPGSGPSSPNSSHS |
| | | TIAENGFTGSVPNIPTEMLPQHRALPLDSSPNQFSLYTSPSLPNISLGL |
| | | QATVTVTNSHLTASPKLSTQQEAERQALQSLRQGGTLTGKFMSTSSI |
| | | PGCLLGVALEGDGSPHGHASLLQHVLLLEQARQQSTLIAVPLHGQSP |
| | | LVTGERVATSMRTVGKLPRHRPLSRTQSSPLPQSPQALQQLVMQQQ |
| | | HQQFLEKQKQQQLQLGKILTKTGELPRQPTTHPEETEEELTEQQEVL |
| | | LGEGALTMPREGSTESESTQEDLEEEDEEDDGEEEEDCIQVKDEEGE |
| | | SGAEEGPDLEEPGAGYKKLFSDAQPLQPLQVYQAPLSLATVPHQAL |
| | | GRTQSSPAAPGGMKSPPDQPVKHLFTTGVVYDTFMLKHQCMCGNT |
| | | HVHPEHAGRIQSIWSRLQETGLLSKCERIRGRKATLDEIQTVHSEYHT |
| | | LLYGTSPLNRQKLDSKKLLGPISQKMYAVLPCGGIGVDSDTVWNEM |
| | | HSSSAVRMAVGCLLELAFKVAAGELKNGFAIIRPPGHHAEESTAMGF |
| | | CFFNSVAITAKLLQQKLNVGKVLIVDWDIHHGNGTQQAFYNDPSVL |
| | | YISLHRYDNGNFFPGSGAPEEVGGGPGVGYNVNVAWTGGVDPPIGD |
| | | VEYLTAFRTVVMPIAHEFSPDVVLVSAGFDAVEGHLSPLGGYSVTAR |
| | | CFGHLTRQLMTLAGGRVVLALEGGHDLTAICDASEACVSALLSVEL |
| | | QPLDEAVLQQKPNINAVATLEKVIEIQSKHWSCVQKFAAGLGRSLRE |
| | | AQAGETEEAETVSAMALLSVGAEQAQAAAAREHSPRPAEEPMEQEP |
| | | AL |
| 1112 | (Hs)DNMT1- | MPARTAPARVPTLAVPAISLPDDVRRRLKDLERDSLTEKECVKEKLN |
| | Cas | LLHEFLQTEIKNQLCDLETKLRKEELSEEGYLAKVKSLLNKDLSLEN |
| | | GAHAYNREVNGRLENGNQARSEARRVGMADANSPPKPLSKPRTPR |
| | | RSKSDGEAKPEPSPSPRITRKSTRQTTITSHFAKGPAKRKPQEESERAK |
| | | SDESIKEEDKDQDEKRRRVTSRERVARPLPAEEPERAKSGTRTEKEEE |
| | | RDEKEEKRLRSQTKEPTPKQKLKEEPDREARAGVQADEDEDGDEKD |
| | | EKKHRSQPKDLAAKRRPEEKEPEKVNPQISDEKDEDEKEEKRRKTTP |
| | | KEPTEKKMARAKTVMNSKTHPPKCIQCGQYLDDPLKYGQHPPDAV |
| | | DEPQMLTNEKLSIFDANESGFESYEALPQHKLTCFSVYCKHGHLCPID |
| | | TGLIEKNIELFFSGSAKPIYDDDPSLEGGVNGKNLGPINEWWITGFDG |
| | | GEKALIGFSTSFAEYILMDPSPEYAPIFGLMQEKIYISKIVVEFLQSNSD |
| | | STYEDLINKIETTVPPSGLNLNRFTEDSLLRHAQFVVEQVESYDEAGD |
| | | SDEQPIFLTPCMRDLIKLAGVTLGQRRAQARRQTIRHSTREKDRGPT |
| | | KATTTKLVYQIFDTFFAEQIEKDDREDKENAFKRRRCGVCEVCQQPE |
| | | CGKCKACKDMVKFGGSGRSKQACQERRCPNMAMKEADDDEEVDD |
| | | NIPEMPSPKKMHQGKKKKQNKNRISWVGEAVKTDGKKSYYKKVCI |
| | | DAETLEVGDCVSVIPDDSSKPLYLARVTALWEDSSNGQMFHAHWFC |
| | | AGTDTVLGATSDPLELFLVDECEDMQLSYIHSKVKVIYKAPSENWA |
| | | MEGGMDPESLLEGDDGKTYFYQLWYDQDYARFESPPKTQPTEDNK |
| | | FKFCVSCARLAEMRQKEIPRVLEQLEDLDSRVLYYSATKNGILYRVG |
| | | DGVYLPPEAFTFNIKLSSPVKRPRKEPVDEDLYPEHYRKYSDYIKGSN |
| | | LDAPEPYRIGRIKEIFCPKKSNGRPNETDIKIRVNKFYRPENTHKSTPA |
| | | SYHADINLLYWSDEEAVVDFKAVQGRCTVEYGEDLPECVQVYSMG |
| | | GPNRFYFLEAYNAKSKSFEDPPNHARSPGNKGKGKGKGKGKPKSQA |
| | | CEPSEPEIEIKLPKLRTLDVFSGCGGLSEGFHQAGISDTLWAIEMWDP |
| | | AAQAFRLNNPGSTVFTEDCNILLKLVMAGETTNSRGQRLPQKGDVE |
| | | MLCGGPPCQGFSGMNRFNSRTYSKFKNSLVVSFLSYCDYYRPRFFLL |
| | | ENVRNFVSFKRSMVLKLTLRCLVRMGYQCTFGVLQAGQYGVAQTR |
| | | RRAIILAAAPGEKLPLFPEPLHVFAPRACQLSVVVDDKKFVSNITRLS |
| | | SGPFRTITVRDTMSDLPEVRNGASALEISYNGEPQSWFQRQLRGAQY |
| | | QPILRDHICKDMSALVAARMRHIPLAPGSDWRDLPNIEVRLSDGTMA |
| | | RKLRYTHHDRKNGRSSSGALRGVCSCVEAGKACDPAARQFNTLIPW |
| | | CLPHTGNRHNHWAGLYGRLEWDGFFSTTVTNPEPMGKQGRVLHPE |
| | | QHRVVSVRECARSQGFPDTYRLFGNILDKHRQVGNAVPPPLAKAIGL |
| | | EIKLCMLAKARESASAKIKEEEAAKDGGPSSGAPPPSGGSPAGSPTST |
| | | EEGTSESATPESGPGTSTEPSEGSAPGSPAGSPTSTEEGTSTEPSEGSAP |
| | | GTSTEPSEPKKKRKVMDKKYSIGLAIGTNSVGWAVITDEYKVPSKKF |
| | | KVLGNTDRHSIKKNLIGALLFDSGETAEATRLKRTARRRYTRRKNRI |
| | | CYLQEIFSNEMAKVDDSFFHRLEESFLVEEDKKHERHPIFGNIVDEVA |
| | | YHEKYPTIYHLRKKLVDSTDKADLRLIYLALAHMIKFRGHFLIEGDL |
| | | NPDNSDVDKLFIQLVQTYNQLFEENPINASGVDAKAILSARLSKSRRL |
| | | ENLIAQLPGEKKNGLFGNLIALSLGLTPNFKSNFDLAEDAKLQLSKDT |
| | | YDDDLDNLLAQIGDQYADLFLAAKNLSDAILLSDILRVNTEITKAPLS |
| | | ASMIKRYDEHHQDLTLLKALVRQQLPEKYKEIFFDQSKNGYAGYID |
| | | GGASQEEFYKFIKPILEKMDGTEELLVKLNREDLLRKQRTFDNGSIPH |
| | | QIHLGELHAILRRQEDFYPFLKDNREKIEKILTFRIPYYVGPLARGNSR |
| | | FAWMTRKSEETITPWNFEEVVDKGASAQSFIERMTNFDKNLPNEKV |
| | | LPKHSLLYEYFTVYNELTKVKYVTEGMRKPAFLSGEQKKAIVDLLF |
| | | KTNRKVTVKQLKEDYFKKIECFDSVEISGVEDRFNASLGTYHDLLKII |
| | | KDKDFLDNEENEDILEDIVLTLTLFEDREMIEERLKTYAHLFDDKVM |
| | | KQLKRRRYTGWGRLSRKLINGIRDKQSGKTILDFLKSDGFANRNFM |
| | | QLIHDDSLTFKEDIQKAQVSGQGDSLHEHIANLAGSPAIKKGILQTVK |
| | | VVDELVKVMGRHKPENIVIEMARENQTTQKGQKNSRERMKRIEEGI |
| | | KELGSQILKEHPVENTQLQNEKLYLYYLQNGRDMYVDQELDINRLS |
| | | DYDVDAIVPQSFLKDDSIDNKVLTRSDKNRGKSDNVPSEEVVKKMK |
| | | NYWRQLLNAKLITQRKFDNLTKAERGGLSELDKAGFIKRQLVETRQI |
| | | TKHVAQILDSRMNTKYDENDKLIREVKVITLKSKLVSDFRKDFQFYK |
| | | VREINNYHHAHDAYLNAVVGTALIKKYPKLESEFVYGDYKVYDVR |
| | | KMIAKSEQEIGKATAKYFFYSNIMNFFKTEITLANGEIRKRPLIETNGE |
| | | TGEIVWDKGRDFATVRKVLSMPQVNIVKKTEVQTGGFSKESILPKRN |
| | | SDKLIARKKDWDPKKYGGFDSPTVAYSVLVVAKVEKGKSKKLKSV |
| | | KELLGITIMERSSFEKNPIDFLEAKGYKEVKKDLIIKLPKYSLFELENG |
| | | RKRMLASAGELQKGNELALPSKYVNFLYLASHYEKLKGSPEDNEQK |
| | | QLFVEQHKHYLDEIIEQISEFSKRVILADANLDKVLSAYNKHRDKPIR |
| | | EQAENIIHLFTLTNLGAPAAFKYFDTTIDRKRYTSTKEVLDATLIHQSI |
| | | TGLYETRIDLSQLGGDPKKKRKV |
| 1113 | (Hs)DNMT3A- | MPAMPSSGPGDTSSSAAEREEDRKDGEEQEEPRGKEERQEPSTTARK |
| | Cas | VGRPGRKRKHPPVESGDTPKDPAVISKSPSMAQDSGASELLPNGDLE |
| | | KRSEPQPEEGSPAGGQKGGAPAEGEGAAETLPEASRAVENGCCTPKE |
| | | GRGAPAEAGKEQKETNIESMKMEGSRGRLRGGLGWESSLRQRPMPR |
| | | LTFQAGDPYYISKRKRDEWLARWKREAEKKAKVIAGMNAVEENQG |
| | | PGESQKVEEASPPAVQQPTDPASPTVATTPEPVGSDAGDKNATKAG |
| | | DDEPEYEDGRGFGIGELVWGKLRGFSWWPGRIVSWWMTGRSRAAE |
| | | GTRWVMWFGDGKFSVVCVEKLMPLSSFCSAFHQATYNKQPMYRK |
| | | AIYEVLQVASSRAGKLFPVCHDSDESDTAKAVEVQNKPMIEWALGG |
| | | FQPSGPKGLEPPEEEKNPYKEVYTDMWVEPEAAAYAPPPPAKKPRK |
| | | STAEKPKVKEIIDERTRERLVYEVRQKCRNIEDICISCGSLNVTLEHPL |
| | | FVGGMCQNCKNCFLECAYQYDDDGYQSYCTICCGGREVLMCGNNN |
| | | CCRCFCVECVDLLVGPGAAQAAIKEDPWNCYMCGHKGTYGLLRRR |
| | | EDWPSRLQMFFANNHDQEFDPPKVYPPVPAEKRKPIRVLSLFDGIAT |
| | | GLLVLKDLGIQVDRYIASEVCEDSITVGMVRHQGKIMYVGDVRSVT |
| | | QKHIQEWGPFDLVIGGSPCNDLSIVNPARKGLYEGTGRLFFEFYRLLH |
| | | DARPKEGDDRPFFWLFENVVAMGVSDKRDISRFLESNPVMIDAKEV |
| | | SAAHRARYFWGNLPGMNRPLASTVNDKLELQECLEHGRIAKFSKVR |
| | | TITTRSNSIKQGKDQHFPVFMNEKEDILWCTEMERVFGFPVHYTDVS |
| | | NMSRLARQRLLGRSWSVPVIRHLFAPLKEYFACVGGPSSGAPPPSGG |
| | | SPAGSPTSTEEGTSESATPESGPGTSTEPSEGSAPGSPAGSPTSTEEGTS |
| | | TEPSEGSAPGTSTEPSEPKKKRKVMDKKYSIGLAIGTNSVGWAVITD |
| | | EYKVPSKKFKVLGNTDRHSIKKNLIGALLFDSGETAEATRLKRTARR |
| | | RYTRRKNRICYLQEIFSNEMAKVDDSFFHRLEESFLVEEDKKHERHPI |
| | | FGNIVDEVAYHEKYPTIYHLRKKLVDSTDKADLRLIYLALAHMIKFR |
| | | GHFLIEGDLNPDNSDVDKLFIQLVQTYNQLFEENPINASGVDAKAILS |
| | | ARLSKSRRLENLIAQLPGEKKNGLFGNLIALSLGLTPNFKSNFDLAED |
| | | AKLQLSKDTYDDDLDNLLAQIGDQYADLFLAAKNLSDAILLSDILRV |
| | | NTEITKAPLSASMIKRYDEHHQDLTLLKALVRQQLPEKYKEIFFDQS |
| | | KNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYY |
| | | VGPLARGNSRFAWMTRKSEETITPWNFEEVVDKGASAQSFIERMTN |
| | | FDKNLPNEKVLPKHSLLYEYFTVYNELTKVKYVTEGMRKPAFLSGE |
| | | QKKAIVDLLFKTNRKVTVKQLKEDYFKKIECFDSVEISGVEDRFNAS |
| | | LGTYHDLLKIIKDKDFLDNEENEDILEDIVLTLTLFEDREMIEERLKTY |
| | | AHLFDDKVMKQLKRRRYTGWGRLSRKLINGIRDKQSGKTILDFLKS |
| | | DGFANRNFMQLIHDDSLTFKEDIQKAQVSGQGDSLHEHIANLAGSPA |
| | | IKKGILQTVKVVDELVKVMGRHKPENIVIEMARENQTTQKGQKNSR |
| | | ERMKRIEEGIKELGSQILKEHPVENTQLQNEKLYLYYLQNGRDMYV |
| | | DQELDINRLSDYDVDAIVPQSFLKDDSIDNKVLTRSDKNRGKSDNVP |
| | | SEEVVKKMKNYWRQLLNAKLITQRKFDNLTKAERGGLSELDKAGFI |
| | | KRQLVETRQITKHVAQILDSRMNTKYDENDKLIREVKVITLKSKLVS |
| | | DFRKDFQFYKVREINNYHHAHDAYLNAVVGTALIKKYPKLESEFVY |
| | | GDYKVYDVRKMIAKSEQEIGKATAKYFFYSNIMNFFKTEITLANGEI |
| | | RKRPLIETNGETGEIVWDKGRDFATVRKVLSMPQVNIVKKTEVQTG |
| | | GFSKESILPKRNSDKLIARKKDWDPKKYGGFDSPTVAYSVLVVAKV |
| | | EKGKSKKLKSVKELLGITIMERSSFEKNPIDFLEAKGYKEVKKDLIIK |
| | | LPKYSLFELENGRKRMLASAGELQKGNELALPSKYVNFLYLASHYE |
| | | KLKGSPEDNEQKQLFVEQHKHYLDEIIEQISEFSKRVILADANLDKVL |
| | | SAYNKHRDKPIREQAENIIHLFTLTNLGAPAAFKYFDTTIDRKRYTST |
| | | KEVLDATLIHQSITGLYETRIDLSQLGGDPKKKRKV |
| 1114 | (Hs)DNMT3A | MNHDQEFDPPKVYPPVPAEKRKPIRVLSLFDGIATGLLVLKDLGIQV |
| | (CD)-Cas | DRYIASEVCEDSITVGMVRHQGKIMYVGDVRSVTQKHIQEWGPFDL |
| | | VIGGSPCNDLSIVNPARKGLYEGTGRLFFEFYRLLHDARPKEGDDRPF |
| | | FWLFENVVAMGVSDKRDISRFLESNPVMIDAKEVSAAHRARYFWG |
| | | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVGGPSSGAPPPSGGSPAGSPTSTEEGT |
| | | SESATPESGPGTSTEPSEGSAPGSPAGSPTSTEEGTSTEPSEGSAPGTST |
| | | EPSEPKKKRKVMDKKYSIGLAIGTNSVGWAVITDEYKVPSKKFKVL |
| | | GNTDRHSIKKNLIGALLFDSGETAEATRLKRTARRRYTRRKNRICYL |
| | | QEIFSNEMAKVDDSFFHRLEESFLVEEDKKHERHPIFGNIVDEVAYHE |
| | | KYPTIYHLRKKLVDSTDKADLRLIYLALAHMIKFRGHFLIEGDLNPD |
| | | NSDVDKLFIQLVQTYNQLFEENPINASGVDAKAILSARLSKSRRLENL |
| | | IAQLPGEKKNGLFGNLIALSLGLTPNFKSNFDLAEDAKLQLSKDTYD |
| | | DDLDNLLAQIGDQYADLFLAAKNLSDAILLSDILRVNTEITKAPLSAS |
| | | MIKRYDEHHQDLTLLKALVRQQLPEKYKEIFFDQSKNGYAGYIDGG |
| | | ASQEEFYKFIKPILEKMDGTEELLVKLNREDLLRKQRTFDNGSIPHQI |
| | | HLGELHAILRRQEDFYPFLKDNREKIEKILTFRIPYYVGPLARGNSRF |
| | | AWMTRKSEETITPWNFEEVVDKGASAQSFIERMTNFDKNLPNEKVL |
| | | PKHSLLYEYFTVYNELTKVKYVTEGMRKPAFLSGEQKKAIVDLLFK |
| | | TNRKVTVKQLKEDYFKKIECFDSVEISGVEDRFNASLGTYHDLLKIIK |
| | | DKDFLDNEENEDILEDIVLTLTLFEDREMIEERLKTYAHLFDDKVMK |
| | | QLKRRRYTGWGRLSRKLINGIRDKQSGKTILDFLKSDGFANRNFMQL |
| | | IHDDSLTFKEDIQKAQVSGQGDSLHEHIANLAGSPAIKKGILQTVKVV |
| | | DELVKVMGRHKPENIVIEMARENQTTQKGQKNSRERMKRIEEGIKE |
| | | LGSQILKEHPVENTQLQNEKLYLYYLQNGRDMYVDQELDINRLSDY |
| | | DVDAIVPQSFLKDDSIDNKVLTRSDKNRGKSDNVPSEEVVKKMKNY |
| | | WRQLLNAKLITQRKFDNLTKAERGGLSELDKAGFIKRQLVETRQITK |
| | | HVAQILDSRMNTKYDENDKLIREVKVITLKSKLVSDFRKDFQFYKVR |
| | | EINNYHHAHDAYLNAVVGTALIKKYPKLESEFVYGDYKVYDVRKMI |
| | | AKSEQEIGKATAKYFFYSNIMNFFKTEITLANGEIRKRPLIETNGETGE |
| | | IVWDKGRDFATVRKVLSMPQVNIVKKTEVQTGGFSKESILPKRNSDK |
| | | LIARKKDWDPKKYGGFDSPTVAYSVLVVAKVEKGKSKKLKSVKEL |
| | | LGITIMERSSFEKNPIDFLEAKGYKEVKKDLIIKLPKYSLFELENGRKR |
| | | MLASAGELQKGNELALPSKYVNFLYLASHYEKLKGSPEDNEQKQLF |
| | | VEQHKHYLDEIIEQISEFSKRVILADANLDKVLSAYNKHRDKPIREQA |
| | | ENIIHLFTLTNLGAPAAFKYFDTTIDRKRYTSTKEVLDATLIHQSITGL |
| | | YETRIDLSQLGGDPKKKRKV |
| 1115 | (Hs/Hs) | MNHDQEFDPPKVYPPVPAEKRKPIRVLSLFDGIATGLLVLKDLGIQV |
| | DNMT3A(CD)/ | DRYIASEVCEDSITVGMVRHQGKIMYVGDVRSVTQKHIQEWGPFDL |
| | L(CD)-Cas | VIGGSPCNDLSIVNPARKGLYEGTGRLFFEFYRLLHDARPKEGDDRPF |
| | | FWLFENVVAMGVSDKRDISRFLESNPVMIDAKEVSAAHRARYFWG |
| | | NLPGMNRPLASTVNDKLELQECLEHGRIAKFSKVRTITTRSNSIKQGK |
| | | DQHFPVFMNEKEDILWCTEMERVFGFPVHYTDVSNMSRLARQRLLG |
| | | RSWSVPVIRHLFAPLKEYFACVSSGNSNANSRGPSFSSGLVPLSLRGS |
| | | HNPLEMFETVPVWRRQPVRVLSLFEDIKKELTSLGFLESGSDPGQLK |
| | | HVVDVTDTVRKDVEEWGPFDLVYGATPPLGHTCDRPPSWYLFQFH |
| | | RLLQYARPKPGSPRPFFWMFVDNLVLNKEDLDVASRFLEMEPVTIPD |
| | | VHGGSLQNAVRVWSNIPAIRSRHWALVSEEELSLLAQNKQSSKLAA |
| | | KWPTKLVKNCFLPLREYFKYFSTELTSSLGGPSSGAPPPSGGSPAGSP |
| | | TSTEEGTSESATPESGPGTSTEPSEGSAPGSPAGSPTSTEEGTSTEPSEG |
| | | SAPGTSTEPSEPKKKRKVMDKKYSIGLAIGTNSVGWAVITDEYKVPS |
| | | KKFKVLGNTDRHSIKKNLIGALLFDSGETAEATRLKRTARRRYTRRK |
| | | NRICYLQEIFSNEMAKVDDSFFHRLEESFLVEEDKKHERHPIFGNIVD |
| | | EVAYHEKYPTIYHLRKKLVDSTDKADLRLIYLALAHMIKFRGHFLIE |
| | | GDLNPDNSDVDKLFIQLVQTYNQLFEENPINASGVDAKAILSARLSK |
| | | SRRLENLIAQLPGEKKNGLFGNLIALSLGLTPNFKSNFDLAEDAKLQL |
| | | SKDTYDDDLDNLLAQIGDQYADLFLAAKNLSDAILLSDILRVNTEIT |
| | | KAPLSASMIKRYDEHHQDLTLLKALVRQQLPEKYKEIFFDQSKNGY |
| | | AGYIDGGASQEEFYKFIKPILEKMDGTEELLVKLNREDLLRKQRTFD |
| | | NGSIPHQIHLGELHAILRRQEDFYPFLKDNREKIEKILTFRIPYYVGPL |
| | | ARGNSRFAWMTRKSEETITPWNFEEVVDKGASAQSFIERMTNFDKN |
| | | LPNEKVLPKHSLLYEYFTVYNELTKVKYVTEGMRKPAFLSGEQKKA |
| | | IVDLLFKTNRKVTVKQLKEDYFKKIECFDSVEISGVEDRFNASLGTY |
| | | HDLLKIIKDKDFLDNEENEDILEDIVLTLTLFEDREMIEERLKTYAHLF |
| | | DDKVMKQLKRRRYTGWGRLSRKLINGIRDKQSGKTILDFLKSDGFA |
| | | NRNFMQLIHDDSLTFKEDIQKAQVSGQGDSLHEHIANLAGSPAIKKGI |
| | | LQTVKVVDELVKVMGRHKPENIVIEMARENQTTQKGQKNSRERMK |
| | | RIEEGIKELGSQILKEHPVENTQLQNEKLYLYYLQNGRDMYVDQELD |
| | | INRLSDYDVDAIVPQSFLKDDSIDNKVLTRSDKNRGKSDNVPSEEVV |
| | | KKMKNYWRQLLNAKLITQRKFDNLTKAERGGLSELDKAGFIKRQLV |
| | | ETRQITKHVAQILDSRMNTKYDENDKLIREVKVITLKSKLVSDFRKD |
| | | FQFYKVREINNYHHAHDAYLNAVVGTALIKKYPKLESEFVYGDYKV |
| | | YDVRKMIAKSEQEIGKATAKYFFYSNIMNFFKTEITLANGEIRKRPLI |
| | | ETNGETGEIVWDKGRDFATVRKVLSMPQVNIVKKTEVQTGGFSKESI |
| | | LPKRNSDKLIARKKDWDPKKYGGFDSPTVAYSVLVVAKVEKGKSK |
| | | KLKSVKELLGITIMERSSFEKNPIDFLEAKGYKEVKKDLIIKLPKYSLF |
| | | ELENGRKRMLASAGELQKGNELALPSKYVNFLYLASHYEKLKGSPE |
| | | DNEQKQLFVEQHKHYLDEIIEQISEFSKRVILADANLDKVLSAYNKH |
| | | RDKPIREQAENIIHLFTLTNLGAPAAFKYFDTTIDRKRYTSTKEVLDA |
| | | TLIHQSITGLYETRIDLSQLGGDPKKKRKV |
| 1116 | (Hs)DNMT3B- | MKGDTRHLNGEEDAGGREDSILVNGACSDQSSDSPPILEAIRTPEIRG |
| | Cas | RRSSSRLSKREVSSLLSYTQDLTGDGDGEDGDGSDTPVMPKLFRETR |
| | | TRSESPAVRTRNNNSVSSRERHRPSPRSTRGRQGRNHVDESPVEFPAT |
| | | RSLRRRATASAGTPWPSPPSSYLTIDLTDDTEDTHGTPQSSSTPYARL |
| | | AQDSQQGGMESPQVEADSGDGDSSEYQDGKEFGIGDLVWGKIKGFS |
| | | WWPAMVVSWKATSKRQAMSGMRWVQWFGDGKFSEVSADKLVAL |
| | | GLFSQHFNLATFNKLVSYRKAMYHALEKARVRAGKTFPSSPGDSLE |
| | | DQLKPMLEWAHGGFKPTGIEGLKPNNTQPVVNKSKVRRAGSRKLES |
| | | RKYENKTRRRTADDSATSDYCPAPKRLKTNCYNNGKDRGDEDQSR |
| | | EQMASDVANNKSSLEDGCLSCGRKNPVSFHPLFEGGLCQTCRDRFL |
| | | ELFYMYDDDGYQSYCTVCCEGRELLLCSNTSCCRCFCVECLEVLVG |
| | | TGTAAEAKLQEPWSCYMCLPQRCHGVLRRRKDWNVRLQAFFTSDT |
| | | GLEYEAPKLYPAIPAARRRPIRVLSLFDGIATGYLVLKELGIKVGKYV |
| | | ASEVCEESIAVGTVKHEGNIKYVNDVRNITKKNIEEWGPFDLVIGGSP |
| | | CNDLSNVNPARKGLYEGTGRLFFEFYHLLNYSRPKEGDDRPFFWMF |
| | | ENVVAMKVGDKRDISRFLECNPVMIDAIKVSAAHRARYFWGNLPG |
| | | MNRPVIASKNDKLELQDCLEYNRIAKLKKVQTITTKSNSIKQGKNQL |
| | | FPVVMNGKEDVLWCTELERIFGFPVHYTDVSNMGRGARQKLLGRS |
| | | WSVPVIRHLFAPLKDYFACEGGPSSGAPPPSGGSPAGSPTSTEEGTSE |
| | | SATPESGPGTSTEPSEGSAPGSPAGSPTSTEEGTSTEPSEGSAPGTSTEP |
| | | SEPKKKRKVMDKKYSIGLAIGTNSVGWAVITDEYKVPSKKFKVLGN |
| | | TDRHSIKKNLIGALLFDSGETAEATRLKRTARRRYTRRKNRICYLQEI |
| | | FSNEMAKVDDSFFHRLEESFLVEEDKKHERHPIFGNIVDEVAYHEKY |
| | | PTIYHLRKKLVDSTDKADLRLIYLALAHMIKFRGHFLIEGDLNPDNSD |
| | | VDKLFIQLVQTYNQLFEENPINASGVDAKAILSARLSKSRRLENLIAQ |
| | | LPGEKKNGLFGNLIALSLGLTPNFKSNFDLAEDAKLQLSKDTYDDDL |
| | | DNLLAQIGDQYADLFLAAKNLSDAILLSDILRVNTEITKAPLSASMIK |
| | | RYDEHHQDLTLLKALVRQQLPEKYKEIFFDQSKNGYAGYIDGGASQ |
| | | EEFYKFIKPILEKMDGTEELLVKLNREDLLRKQRTFDNGSIPHQIHLG |
| | | ELHAILRRQEDFYPFLKDNREKIEKILTFRIPYYVGPLARGNSRFAWM |
| | | TRKSEETITPWNFEEVVDKGASAQSFIERMTNFDKNLPNEKVLPKHS |
| | | LLYEYFTVYNELTKVKYVTEGMRKPAFLSGEQKKAIVDLLFKTNRK |
| | | VTVKQLKEDYFKKIECFDSVEISGVEDRFNASLGTYHDLLKIIKDKDF |
| | | LDNEENEDILEDIVLTLTLFEDREMIEERLKTYAHLFDDKVMKQLKR |
| | | RRYTGWGRLSRKLINGIRDKQSGKTILDFLKSDGFANRNFMQLIHDD |
| | | SLTFKEDIQKAQVSGQGDSLHEHIANLAGSPAIKKGILQTVKVVDEL |
| | | VKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKV |
| 1117 | (Hs)DNMT3B | MIRVLSLFDGIATGYLVLKELGIKVGKYVASEVCEESIAVGTVKHEG |
| | (CD)-Cas | NIKYVNDVRNITKKNIEEWGPFDLVIGGSPCNDLSNVNPARKGLYEG |
| | | TGRLFFEFYHLLNYSRPKEGDDRPFFWMFENVVAMKVGDKRDISRF |
| | | LECNPVMIDAIKVSAAHRARYFWGNLPGMNRPVIASKNDKLELQDC |
| | | LEYNRIAKLKKVQTITTKSNSIKQGKNQLFPVVMNGKEDVLWCTEL |
| | | ERIFGFPVHYTDVSNMGRGARQKLLGRSWSVPVIRHLFAPLKDYFAC |
| | | EGGPSSGAPPPSGGSPAGSPTSTEEGTSESATPESGPGTSTEPSEGSAP |
| | | GSPAGSPTSTEEGTSTEPSEGSAPGTSTEPSEPKKKRKVMDKKYSIGL |
| | | AIGTNSVGWAVITDEYKVPSKKFKVLGNTDRHSIKKNLIGALLFDSG |
| | | ETAEATRLKRTARRRYTRRKNRICYLQEIFSNEMAKVDDSFFHRLEE |
| | | SFLVEEDKKHERHPIFGNIVDEVAYHEKYPTIYHLRKKLVDSTDKAD |
| | | LRLIYLALAHMIKFRGHFLIEGDLNPDNSDVDKLFIQLVQTYNQLFEE |
| | | NPINASGVDAKAILSARLSKSRRLENLIAQLPGEKKNGLFGNLIALSL |
| | | GLTPNFKSNFDLAEDAKLQLSKDTYDDDLDNLLAQIGDQYADLFLA |
| | | AKNLSDAILLSDILRVNTEITKAPLSASMIKRYDEHHQDLTLLKALVR |
| | | QQLPEKYKEIFFDQSKNGYAGYIDGGASQEEFYKFIKPILEKMDGTEE |
| | | LLVKLNREDLLRKQRTFDNGSIPHQIHLGELHAILRRQEDFYPFLKDN |
| | | REKIEKILTFRIPYYVGPLARGNSRFAWMTRKSEETITPWNFEEVVDK |
| | | GASAQSFIERMTNFDKNLPNEKVLPKHSLLYEYFTVYNELTKVKYVT |
| | | EGMRKPAFLSGEQKKAIVDLLFKTNRKVTVKQLKEDYFKKIECFDSV |
| | | EISGVEDRFNASLGTYHDLLKIIKDKDFLDNEENEDILEDIVLTLTLFE |
| | | DREMIEERLKTYAHLFDDKVMKQLKRRRYTGWGRLSRKLINGIRDK |
| | | QSGKTILDFLKSDGFANRNFMQLIHDDSLTFKEDIQKAQVSGQGDSL |
| | | HEHIANLAGSPAIKKGILQTVKVVDELVKVMGRHKPENIVIEMAREN |
| | | QTTQKGQKNSRERMKRIEEGIKELGSQILKEHPVENTQLQNEKLYLY |
| | | YLQNGRDMYVDQELDINRLSDYDVDAIVPQSFLKDDSIDNKVLTRS |
| | | DKNRGKSDNVPSEEVVKKMKNYWRQLLNAKLITQRKFDNLTKAER |
| | | GGLSELDKAGFIKRQLVETRQITKHVAQILDSRMNTKYDENDKLIRE |
| | | VKVITLKSKLVSDFRKDFQFYKVREINNYHHAHDAYLNAVVGTALI |
| | | KKYPKLESEFVYGDYKVYDVRKMIAKSEQEIGKATAKYFFYSNIMN |
| | | FFKTEITLANGEIRKRPLIETNGETGEIVWDKGRDFATVRKVLSMPQV |
| | | NIVKKTEVQTGGFSKESILPKRNSDKLIARKKDWDPKKYGGFDSPTV |
| | | AYSVLVVAKVEKGKSKKLKSVKELLGITIMERSSFEKNPIDFLEAKG |
| | | YKEVKKDLIIKLPKYSLFELENGRKRMLASAGELQKGNELALPSKYV |
| | | NFLYLASHYEKLKGSPEDNEQKQLFVEQHKHYLDEIIEQISEFSKRVI |
| | | LADANLDKVLSAYNKHRDKPIREQAENIIHLFTLTNLGAPAAFKYFD |
| | | TTIDRKRYTSTKEVLDATLIHQSITGLYETRIDLSQLGGDPKKKRKV |
| 1118 | (Hs/Mm) | MIRVLSLFDGIATGYLVLKELGIKVGKYVASEVCEESIAVGTVKHEG |
| | DNMT3B(CD)/ | NIKYVNDVRNITKKNIEEWGPFDLVIGGSPCNDLSNVNPARKGLYEG |
| | L(CD)-Cas | TGRLFFEFYHLLNYSRPKEGDDRPFFWMFENVVAMKVGDKRDISRF |
| | | LECNPVMIDAIKVSAAHRARYFWGNLPGMNRPVIASKNDKLELQDC |
| | | LEYNRIAKLKKVQTITTKSNSIKQGKNQLFPVVMNGKEDVLWCTEL |
| | | ERIFGFPVHYTDVSNMGRGARQKLLGRSWSVPVIRHLFAPLKDYFAC |
| | | ESSGNSNANSRGPSFSSGLVPLSLRGSHMGPMEIYKTVSAWKRQPVR |
| | | VLSLFRNIDKVLKSLGFLESGSGSGGGTLKYVEDVTNVVRRDVEKW |
| | | GPFDLVYGSTQPLGSSCDRCPGWYMFQFHRILQYALPRQESQRPFFW |
| | | IFMDNLLLTEDDQETTTRFLQTEAVTLQDVRGRDYQNAMRVWSNIP |
| | | GLKSKHAPLTPKEEEYLQAQVRSRSKLDAPKVDLLVKNCLLPLREYF |
| | | KYFSQNSLPLGGPSSGAPPPSGGSPAGSPTSTEEGTSESATPESGPGTS |
| | | TEPSEGSAPGSPAGSPTSTEEGTSTEPSEGSAPGTSTEPSEPKKKRKVM |
| | | DKKYSIGLAIGTNSVGWAVITDEYKVPSKKFKVLGNTDRHSIKKNLI |
| | | GALLFDSGETAEATRLKRTARRRYTRRKNRICYLQEIFSNEMAKVDD |
| | | SFFHRLEESFLVEEDKKHERHPIFGNIVDEVAYHEKYPTIYHLRKKLV |
| | | DSTDKADLRLIYLALAHMIKFRGHFLIEGDLNPDNSDVDKLFIQLVQ |
| | | TYNQLFEENPINASGVDAKAILSARLSKSRRLENLIAQLPGEKKNGLF |
| | | GNLIALSLGLTPNFKSNFDLAEDAKLQLSKDTYDDDLDNLLAQIGDQ |
| | | YADLFLAAKNLSDAILLSDILRVNTEITKAPLSASMIKRYDEHHQDLT |
| | | LLKALVRQQLPEKYKEIFFDQSKNGYAGYIDGGASQEEFYKFIKPILE |
| | | KMDGTEELLVKLNREDLLRKQRTFDNGSIPHQIHLGELHAILRRQED |
| | | FYPFLKDNREKIEKILTFRIPYYVGPLARGNSRFAWMTRKSEETITPW |
| | | NFEEVVDKGASAQSFIERMTNFDKNLPNEKVLPKHSLLYEYFTVYNE |
| | | LTKVKYVTEGMRKPAFLSGEQKKAIVDLLFKTNRKVTVKQLKEDYF |
| | | KKIECFDSVEISGVEDRFNASLGTYHDLLKIIKDKDFLDNEENEDILED |
| | | IVLTLTLFEDREMIEERLKTYAHLFDDKVMKQLKRRRYTGWGRLSR |
| | | KLINGIRDKQSGKTILDFLKSDGFANRNFMQLIHDDSLTFKEDIQKAQ |
| | | VSGQGDSLHEHIANLAGSPAIKKGILQTVKVVDELVKVMGRHKPENI |
| | | VIEMARENQTTQKGQKNSRERMKRIEEGIKELGSQILKEHPVENTQL |
| | | QNEKLYLYYLQNGRDMYVDQELDINRLSDYDVDAIVPQSFLKDDSI |
| | | DNKVLTRSDKNRGKSDNVPSEEVVKKMKNYWRQLLNAKLITQRKF |
| | | DNLTKAERGGLSELDKAGFIKRQLVETRQITKHVAQILDSRMNTKYD |
| | | ENDKLIREVKVITLKSKLVSDFRKDFQFYKVREINNYHHAHDAYLNA |
| | | VVGTALIKKYPKLESEFVYGDYKVYDVRKMIAKSEQEIGKATAKYF |
| | | FYSNIMNFFKTEITLANGEIRKRPLIETNGETGEIVWDKGRDFATVRK |
| | | VLSMPQVNIVKKTEVQTGGFSKESILPKRNSDKLIARKKDWDPKKY |
| | | GGFDSPTVAYSVLVVAKVEKGKSKKLKSVKELLGITIMERSSFEKNPI |
| | | DFLEAKGYKEVKKDLIIKLPKYSLFELENGRKRMLASAGELQKGNEL |
| | | ALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPA |
| | | AFKYFDTTIDRKRYTSTKEVLDATLIHQSITGLYETRIDLSQLGGDPK |
| | | KKRKV |
| 1119 | (Hs)DNMT3- | MAAIPALDPEAEPSMDVILVGSSELSSSVSPGTGRDLIAYEVKANQRN |
| | Cas | IEDICICCGSLQVHTQHPLFEGGICAPCKDKFLDALFLYDDDGYQSYC |
| | | SICCSGETLLICGNPDCTRCYCFECVDSLVGPGTSGKVHAMSNWVCY |
| | | LCLPSSRSGLLQRRRKWRSQLKAFYDRESENPLEMFETVPVWRRQP |
| | | VRVLSLFEDIKKELTSLGFLESGSDPGQLKHVVDVTDTVRKDVEEW |
| | | GPFDLVYGATPPLGHTCDRPPSWYLFQFHRLLQYARPKPGSPRPFFW |
| | | MFVDNLVLNKEDLDVASRFLEMEPVTIPDVHGGSLQNAVRVWSNIP |
| | | AIRSSRHWALVSEEELSLLAQNKQSSKLAAKWPTKLVKNCFLPLREY |
| | | FKYFSTELTSSLGGPSSGAPPPSGGSPAGSPTSTEEGTSESATPESGPGT |
| | | STEPSEGSAPGSPAGSPTSTEEGTSTEPSEGSAPGTSTEPSEPKKKRKV |
| | | MDKKYSIGLAIGTNSVGWAVITDEYKVPSKKFKVLGNTDRHSIKKN |
| | | LIGALLFDSGETAEATRLKRTARRRYTRRKNRICYLQEIFSNEMAKV |
| | | DDSFFHRLEESFLVEEDKKHERHPIFGNIVDEVAYHEKYPTIYHLRKK |
| | | LVDSTDKADLRLIYLALAHMIKFRGHFLIEGDLNPDNSDVDKLFIQL |
| | | VQTYNQLFEENPINASGVDAKAILSARLSKSRRLENLIAQLPGEKKN |
| | | GLFGNLIALSLGLTPNFKSNFDLAEDAKLQLSKDTYDDDLDNLLAQI |
| | | GDQYADLFLAAKNLSDAILLSDILRVNTEITKAPLSASMIKRYDEHH |
| | | QDLTLLKALVRQQLPEKYKEIFFDQSKNGYAGYIDGGASQEEFYKFI |
| | | KPILEKMDGTEELLVKLNREDLLRKQRTFDNGSIPHQIHLGELHAILR |
| | | RQEDFYPFLKDNREKIEKILTFRIPYYVGPLARGNSRFAWMTRKSEET |
| | | ITPWNFEEVVDKGASAQSFIERMTNFDKNLPNEKVLPKHSLLYEYFT |
| | | VYNELTKVKYVTEGMRKPAFLSGEQKKAIVDLLFKTNRKVTVKQLK |
| | | EDYFKKIECFDSVEISGVEDRFNASLGTYHDLLKIIKDKDFLDNEENE |
| | | DILEDIVLTLTLFEDREMIEERLKTYAHLFDDKVMKQLKRRRYTGW |
| | | GRLSRKLINGIRDKQSGKTILDFLKSDGFANRNFMQLIHDDSLTFKED |
| | | IQKAQVSGQGDSLHEHIANLAGSPAIKKGILQTVKVVDELVKVMGR |
| | | HKPENIVIEMARENQTTQKGQKNSRERMKRIEEGIKELGSQILKEHPV |
| | | ENTQLQNEKLYLYYLQNGRDMYVDQELDINRLSDYDVDAIVPQSFL |
| | | KDDSIDNKVLTRSDKNRGKSDNVPSEEVVKKMKNYWRQLLNAKLI |
| | | TQRKFDNLTKAERGGLSELDKAGFIKRQLVETRQITKHVAQILDSRM |
| | | NTKYDENDKLIREVKVITLKSKLVSDFRKDFQFYKVREINNYHHAHD |
| | | AYLNAVVGTALIKKYPKLESEFVYGDYKVYDVRKMIAKSEQEIGKA |
| | | TAKYFFYSNIMNFFKTEITLANGEIRKRPLIETNGETGEIVWDKGRDF |
| | | ATVRKVLSMPQVNIVKKTEVQTGGFSKESILPKRNSDKLIARKKDW |
| | | DPKKYGGFDSPTVAYSVLVVAKVEKGKSKKLKSVKELLGITIMERSS |
| | | FEKNPIDFLEAKGYKEVKKDLIIKLPKYSLFELENGRKRMLASAGEL |
| | | QKGNELALPSKYVNFLYLASHYEKLKGSPEDNEQKQLFVEQHKHYL |
| | | DEIIEQISEFSKRVILADANLDKVLSAYNKHRDKPIREQAENIIHLFTLT |
| | | NLGAPAAFKYFDTTIDRKRYTSTKEVLDATLIHQSITGLYETRIDLSQ |
| | | LGGDPKKKRKV |
| 1120 | (Mm)DNMT3L- | MGSRETPSSCSKTLETLDLETSDSSSPDADSPLEEQWLKSSPALKEDS |
| | Cas | VDVVLEDCKEPLSPSSPPTGREMIRYEVKVNRRSIEDICLCCGTLQVY |
| | | TRHPLFEGGLCAPCKDKFLESLFLYDDDGHQSYCTICCSGGTLFICES |
| | | PDCTRCYCFECVDILVGPGTSERINAMACWVCFLCLPFSRSGLLQRR |
| | | KRWRHQLKAFHDQEGAGPMEIYKTVSAWKRQPVRVLSLFRNIDKV |
| | | LKSLGFLESGSGSGGGTLKYVEDVTNVVRRDVEKWGPFDLVYGSTQ |
| | | PLGSSCDRCPGWYMFQFHRILQYALPRQESQRPFFWIFMDNLLLTED |
| | | DQETTTRFLQTEAVTLQDVRGRDYQNAMRVWSNIPGLKSKHAPLTP |
| | | KEEEYLQAQVRSRSKLDAPKVDLLVKNCLLPLREYFKYFSQNSLPLG |
| | | GPSSGAPPPSGGSPAGSPTSTEEGTSESATPESGPGTSTEPSEGSAPGSP |
| | | AGSPTSTEEGTSTEPSEGSAPGTSTEPSEPKKKRKVMDKKYSIGLAIG |
| | | TNSVGWAVITDEYKVPSKKFKVLGNTDRHSIKKNLIGALLFDSGETA |
| | | EATRLKRTARRRYTRRKNRICYLQEIFSNEMAKVDDSFFHRLEESFL |
| | | VEEDKKHERHPIFGNIVDEVAYHEKYPTIYHLRKKLVDSTDKADLRL |
| | | IYLALAHMIKFRGHFLIEGDLNPDNSDVDKLFIQLVQTYNQLFEENPI |
| | | NASGVDAKAILSARLSKSRRLENLIAQLPGEKKNGLFGNLIALSLGLT |
| | | PNFKSNFDLAEDAKLQLSKDTYDDDLDNLLAQIGDQYADLFLAAKN |
| | | LSDAILLSDILRVNTEITKAPLSASMIKRYDEHHQDLTLLKALVRQQL |
| | | PEKYKEIFFDQSKNGYAGYIDGGASQEEFYKFIKPILEKMDGTEELLV |
| | | KLNREDLLRKQRTFDNGSIPHQIHLGELHAILRRQEDFYPFLKDNREK |
| | | IEKILTFRIPYYVGPLARGNSRFAWMTRKSEETITPWNFEEVVDKGAS |
| | | AQSFIERMTNFDKNLPNEKVLPKHSLLYEYFTVYNELTKVKYVTEG |
| | | MRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFED |
| | | REMIEERLKTYAHLFDDKVMKQLKRRRYTGWGRLSRKLINGIRDKQ |
| | | SGKTILDFLKSDGFANRNFMQLIHDDSLTFKEDIQKAQVSGQGDSLH |
| | | EHIANLAGSPAIKKGILQTVKVVDELVKVMGRHKPENIVIEMARENQ |
| | | TTQKGQKNSRERMKRIEEGIKELGSQILKEHPVENTQLQNEKLYLYY |
| | | LQNGRDMYVDQELDINRLSDYDVDAIVPQSFLKDDSIDNKVLTRSD |
| | | KNRGKSDNVPSEEVVKKMKNYWRQLLNAKLITQRKFDNLTKAERG |
| | | GLSELDKAGFIKRQLVETRQITKHVAQILDSRMNTKYDENDKLIREV |
| | | KVITLKSKLVSDFRKDFQFYKVREINNYHHAHDAYLNAVVGTALIK |
| | | KYPKLESEFVYGDYKVYDVRKMIAKSEQEIGKATAKYFFYSNIMNFF |
| | | KTEITLANGEIRKRPLIETNGETGEIVWDKGRDFATVRKVLSMPQVNI |
| | | VKKTEVQTGGFSKESILPKRNSDKLIARKKDWDPKKYGGFDSPTVA |
| | | YSVLVVAKVEKGKSKKLKSVKELLGITIMERSSFEKNPIDFLEAKGY |
| | | KEVKKDLIIKLPKYSLFELENGRKRMLASAGELQKGNELALPSKYVN |
| | | FLYLASHYEKLKGSPEDNEQKQLFVEQHKHYLDEIIEQISEFSKRVIL |
| | | ADANLDKVLSAYNKHRDKPIREQAENIIHLFTLTNLGAPAAFKYFDT |
| | | TIDRKRYTSTKEVLDATLIHQSITGLYETRIDLSQLGGDPKKKRKV |
| 1121 | (Mm)DNMT3C- | MRGGSRHLSNEEDVSGCEDCIIISGTCSDQSSDPKTVPLTQVLEAVCT |
| | Cas | VENRGCRTSSQPSKRKASSLISYVQDLTGDGDEDRDGEVGGSSGSGT |
| | | PVMPQLFCETRIPSKTPAPLSWQANTSASTPWLSPASPYPIIDLTDEDV |
| | | IPQSISTPSVDWSQDSHQEGMDTTQVDAESRDGGNIEYQVSADKLLL |
| | | SQSCILAAFYKLVPYRESIYRTLEKARVRAGKACPSSPGESLEDQLKP |
| | | MLEWAHGGFKPTGIEGLKPNKKQPENKSRRRTTNDPAASESSPPKRL |
| | | KTNSYGGKDRGEDEESREQMASDVTNNKGNLEDHCLSCGRKDPVS |
| | | FHPLFEGGLCQSCRDRFLELFYMYDEDGYQSYCTVCCEGRELLLCSN |
| | | TSCCRCFCVECLEVLVGAGTAEDVKLQEPWSCYMCLPQRCHGVLRR |
| | | RKDWNMRLQDFFTTDPDLEEFEPPKLYPAIPAAKRRPIRVLSLFDGIA |
| | | TGYLVLKELGIKVEKYIASEVCAESIAVGTVKHEGQIKYVDDIRNITK |
| | | EHIDEWGPFDLVIGGSPCNDLSCVNPVRKGLFEGTGRLFFEFYRLLN |
| | | YSCPEEEDDRPFFWMFENVVAMEVGDKRDISRFLECNPVMIDAIKVS |
| | | AAHRARYFWGNLPGMNRPVMASKNDKLELQDCLEFSRTAKLKKVQ |
| | | TITTKSNSIRQGKNQLFPVVMNGKDDVLWCTELERIFGFPEHYTDVS |
| | | NMGRGARQKLLGRSWSVPVIRHLFAPLKDHFACEGGPSSGAPPPSG |
| | | GSPAGSPTSTEEGTSESATPESGPGTSTEPSEGSAPGSPAGSPTSTEEGT |
| | | STEPSEGSAPGTSTEPSEPKKKRKVMDKKYSIGLAIGTNSVGWAVITD |
| | | EYKVPSKKFKVLGNTDRHSIKKNLIGALLFDSGETAEATRLKRTARR |
| | | RYTRRKNRICYLQEIFSNEMAKVDDSFFHRLEESFLVEEDKKHERHPI |
| | | FGNIVDEVAYHEKYPTIYHLRKKLVDSTDKADLRLIYLALAHMIKFR |
| | | GHFLIEGDLNPDNSDVDKLFIQLVQTYNQLFEENPINASGVDAKAILS |
| | | ARLSKSRRLENLIAQLPGEKKNGLFGNLIALSLGLTPNFKSNFDLAED |
| | | AKLQLSKDTYDDDLDNLLAQIGDQYADLFLAAKNLSDAILLSDILRV |
| | | NTEITKAPLSASMIKRYDEHHQDLTLLKALVRQQLPEKYKEIFFDQS |
| | | KNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYY |
| | | VGPLARGNSRFAWMTRKSEETITPWNFEEVVDKGASAQSFIERMTN |
| | | FDKNLPNEKVLPKHSLLYEYFTVYNELTKVKYVTEGMRKPAFLSGE |
| | | QKKAIVDLLFKTNRKVTVKQLKEDYFKKIECFDSVEISGVEDRFNAS |
| | | LGTYHDLLKIIKDKDFLDNEENEDILEDIVLTLTLFEDREMIEERLKTY |
| | | AHLFDDKVMKQLKRRRYTGWGRLSRKLINGIRDKQSGKTILDFLKS |
| | | DGFANRNFMQLIHDDSLTFKEDIQKAQVSGQGDSLHEHIANLAGSPA |
| | | IKKGILQTVKVVDELVKVMGRHKPENIVIEMARENQTTQKGQKNSR |
| | | ERMKRIEEGIKELGSQILKEHPVENTQLQNEKLYLYYLQNGRDMYV |
| | | DQELDINRLSDYDVDAIVPQSFLKDDSIDNKVLTRSDKNRGKSDNVP |
| | | SEEVVKKMKNYWRQLLNAKLITQRKFDNLTKAERGGLSELDKAGFI |
| | | KRQLVETRQITKHVAQILDSRMNTKYDENDKLIREVKVITLKSKLVS |
| | | DFRKDFQFYKVREINNYHHAHDAYLNAVVGTALIKKYPKLESEFVY |
| | | GDYKVYDVRKMIAKSEQEIGKATAKYFFYSNIMNFFKTEITLANGEI |
| | | RKRPLIETNGETGEIVWDKGRDFATVRKVLSMPQVNIVKKTEVQTG |
| | | GFSKESILPKRNSDKLIARKKDWDPKKYGGFDSPTVAYSVLVVAKV |
| | | EKGKSKKLKSVKELLGITIMERSSFEKNPIDFLEAKGYKEVKKDLIIK |
| | | LPKYSLFELENGRKRMLASAGELQKGNELALPSKYVNFLYLASHYE |
| | | KLKGSPEDNEQKQLFVEQHKHYLDEIIEQISEFSKRVILADANLDKVL |
| | | SAYNKHRDKPIREQAENIIHLFTLTNLGAPAAFKYFDTTIDRKRYTST |
| | | KEVLDATLIHQSITGLYETRIDLSQLGGDPKKKRKV |
| 1122 | (Mm)DNMT3C | MIRVLSLFDGIATGYLVLKELGIKVEKYIASEVCAESIAVGTVKHEGQ |
| | (CD)-Cas | IKYVDDIRNITKEHIDEWGPFDLVIGGSPCNDLSCVNPVRKGLFEGTG |
| | | RLFFEFYRLLNYSCPEEEDDRPFFWMFENVVAMEVGDKRDISRFLEC |
| | | NPVMIDAIKVSAAHRARYFWGNLPGMNRPVMASKNDKLELQDCLE |
| | | FSRTAKLKKVQTITTKSNSIRQGKNQLFPVVMNGKDDVLWCTELERI |
| | | FGFPEHYTDVSNMGRGARQKLLGRSWSVPVIRHLFAPLKDHFACEG |
| | | GPSSGAPPPSGGSPAGSPTSTEEGTSESATPESGPGTSTEPSEGSAPGSP |
| | | AGSPTSTEEGTSTEPSEGSAPGTSTEPSEPKKKRKVMDKKYSIGLAIG |
| | | TNSVGWAVITDEYKVPSKKFKVLGNTDRHSIKKNLIGALLFDSGETA |
| | | EATRLKRTARRRYTRRKNRICYLQEIFSNEMAKVDDSFFHRLEESFL |
| | | VEEDKKHERHPIFGNIVDEVAYHEKYPTIYHLRKKLVDSTDKADLRL |
| | | IYLALAHMIKFRGHFLIEGDLNPDNSDVDKLFIQLVQTYNQLFEENPI |
| | | NASGVDAKAILSARLSKSRRLENLIAQLPGEKKNGLFGNLIALSLGLT |
| | | PNFKSNFDLAEDAKLQLSKDTYDDDLDNLLAQIGDQYADLFLAAKN |
| | | LSDAILLSDILRVNTEITKAPLSASMIKRYDEHHQDLTLLKALVRQQL |
| | | PEKYKEIFFDQSKNGYAGYIDGGASQEEFYKFIKPILEKMDGTEELLV |
| | | KLNREDLLRKQRTFDNGSIPHQIHLGELHAILRRQEDFYPFLKDNREK |
| | | IEKILTFRIPYYVGPLARGNSRFAWMTRKSEETITPWNFEEVVDKGAS |
| | | AQSFIERMTNFDKNLPNEKVLPKHSLLYEYFTVYNELTKVKYVTEG |
| | | MRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFED |
| | | REMIEERLKTYAHLFDDKVMKQLKRRRYTGWGRLSRKLINGIRDKQ |
| | | SGKTILDFLKSDGFANRNFMQLIHDDSLTFKEDIQKAQVSGQGDSLH |
| | | EHIANLAGSPAIKKGILQTVKVVDELVKVMGRHKPENIVIEMARENQ |
| | | TTQKGQKNSRERMKRIEEGIKELGSQILKEHPVENTQLQNEKLYLYY |
| | | LQNGRDMYVDQELDINRLSDYDVDAIVPQSFLKDDSIDNKVLTRSD |
| | | KNRGKSDNVPSEEVVKKMKNYWRQLLNAKLITQRKFDNLTKAERG |
| | | GLSELDKAGFIKRQLVETRQITKHVAQILDSRMNTKYDENDKLIREV |
| | | KVITLKSKLVSDFRKDFQFYKVREINNYHHAHDAYLNAVVGTALIK |
| | | KYPKLESEFVYGDYKVYDVRKMIAKSEQEIGKATAKYFFYSNIMNFF |
| | | KTEITLANGEIRKRPLIETNGETGEIVWDKGRDFATVRKVLSMPQVNI |
| | | VKKTEVQTGGFSKESILPKRNSDKLIARKKDWDPKKYGGFDSPTVA |
| | | YSVLVVAKVEKGKSKKLKSVKELLGITIMERSSFEKNPIDFLEAKGY |
| | | KEVKKDLIIKLPKYSLFELENGRKRMLASAGELQKGNELALPSKYVN |
| | | FLYLASHYEKLKGSPEDNEQKQLFVEQHKHYLDEIIEQISEFSKRVIL |
| | | ADANLDKVLSAYNKHRDKPIREQAENIIHLFTLTNLGAPAAFKYFDT |
| | | TIDRKRYTSTKEVLDATLIHQSITGLYETRIDLSQLGGDPKKKRKV |
| 1123 | (Mm/Mm) | MIRVLSLFDGIATGYLVLKELGIKVEKYIASEVCAESIAVGTVKHEGQ |
| | DNMT3C(CD)/ | IKYVDDIRNITKEHIDEWGPFDLVIGGSPCNDLSCVNPVRKGLFEGTG |
| | L(CD)-Cas | RLFFEFYRLLNYSCPEEEDDRPFFWMFENVVAMEVGDKRDISRFLEC |
| | | NPVMIDAIKVSAAHRARYFWGNLPGMNRPVMASKNDKLELQDCLE |
| | | FSRTAKLKKVQTITTKSNSIRQGKNQLFPVVMNGKDDVLWCTELERI |
| | | FGFPEHYTDVSNMGRGARQKLLGRSWSVPVIRHLFAPLKDHFACESS |
| | | GNSNANSRGPSFSSGLVPLSLRGSHMGPMEIYKTVSAWKRQPVRVLS |
| | | LFRNIDKVLKSLGFLESGSGSGGGTLKYVEDVTNVVRRDVEKWGPF |
| | | DLVYGSTQPLGSSCDRCPGWYMFQFHRILQYALPRQESQRPFFWIFM |
| | | DNLLLTEDDQETTTRFLQTEAVTLQDVRGRDYQNAMRVWSNIPGLK |
| | | SKHAPLTPKEEEYLQAQVRSRSKLDAPKVDLLVKNCLLPLREYFKYF |
| | | SQNSLPLGGPSSGAPPPSGGSPAGSPTSTEEGTSESATPESGPGTSTEPS |
| | | EGSAPGSPAGSPTSTEEGTSTEPSEGSAPGTSTEPSEPKKKRKVMDKK |
| | | YSIGLAIGTNSVGWAVITDEYKVPSKKFKVLGNTDRHSIKKNLIGALL |
| | | FDSGETAEATRLKRTARRRYTRRKNRICYLQEIFSNEMAKVDDSFFH |
| | | RLEESFLVEEDKKHERHPIFGNIVDEVAYHEKYPTIYHLRKKLVDSTD |
| | | KADLRLIYLALAHMIKFRGHFLIEGDLNPDNSDVDKLFIQLVQTYNQ |
| | | LFEENPINASGVDAKAILSARLSKSRRLENLIAQLPGEKKNGLFGNLI |
| | | ALSLGLTPNFKSNFDLAEDAKLQLSKDTYDDDLDNLLAQIGDQYAD |
| | | LFLAAKNLSDAILLSDILRVNTEITKAPLSASMIKRYDEHHQDLTLLK |
| | | ALVRQQLPEKYKEIFFDQSKNGYAGYIDGGASQEEFYKFIKPILEKM |
| | | DGTEELLVKLNREDLLRKQRTFDNGSIPHQIHLGELHAILRRQEDFYP |
| | | FLKDNREKIEKILTFRIPYYVGPLARGNSRFAWMTRKSEETITPWNFE |
| | | EVVDKGASAQSFIERMTNFDKNLPNEKVLPKHSLLYEYFTVYNELTK |
| | | VKYVTEGMRKPAFLSGEQKKAIVDLLFKTNRKVTVKQLKEDYFKKI |
| | | ECFDSVEISGVEDRFNASLGTYHDLLKIIKDKDFLDNEENEDILEDIVL |
| | | TLTLFEDREMIEERLKTYAHLFDDKVMKQLKRRRYTGWGRLSRKLI |
| | | NGIRDKQSGKTILDFLKSDGFANRNFMQLIHDDSLTFKEDIQKAQVS |
| | | GQGDSLHEHIANLAGSPAIKKGILQTVKVVDELVKVMGRHKPENIVI |
| | | EMARENQTTQKGQKNSRERMKRIEEGIKELGSQILKEHPVENTQLQN |
| | | EKLYLYYLQNGRDMYVDQELDINRLSDYDVDAIVPQSFLKDDSIDN |
| | | KVLTRSDKNRGKSDNVPSEEVVKKMKNYWRQLLNAKLITQRKFDN |
| | | LTKAERGGLSELDKAGFIKRQLVETRQITKHVAQILDSRMNTKYDEN |
| | | DKLIREVKVITLKSKLVSDFRKDFQFYKVREINNYHHAHDAYLNAV |
| | | VGTALIKKYPKLESEFVYGDYKVYDVRKMIAKSEQEIGKATAKYFF |
| | | YSNIMNFFKTEITLANGEIRKRPLIETNGETGEIVWDKGRDFATVRKV |
| | | LSMPQVNIVKKTEVQTGGFSKESILPKRNSDKLIARKKDWDPKKYG |
| | | GFDSPTVAYSVLVVAKVEKGKSKKLKSVKELLGITIMERSSFEKNPID |
| | | FLEAKGYKEVKKDLIIKLPKYSLFELENGRKRMLASAGELQKGNELA |
| | | LPSKYVNFLYLASHYEKLKGSPEDNEQKQLFVEQHKHYLDEIIEQISE |
| | | FSKRVILADANLDKVLSAYNKHRDKPIREQAENIIHLFTLTNLGAPAA |
| | | FKYFDTTIDRKRYTSTKEVLDATLIHQSITGLYETRIDLSQLGGDPKK |
| | | KRKV |
| 1124 | (Mp)M.MpeI- | MNSNKDKIKVIKVFEAFAGIGSQFKALKNIARSKNWEIQHSGMVEW |
| | Cas | FVDAIVSYVAIHSKNFNPKIEQLDKDILSISNDSKMPISEYGIKKINNTI |
| | | KASYLNYAKKHFNNLFDIKKVNKDNFPKNIDIFTYSFPCQDLSVQGL |
| | | QKGIDKELNTRSGLLWEIERILEEIKNSFSKEEMPKYLLMENVKNLLS |
| | | HKNKKNYNTWLKQLEKFGYKSKTYLLNSKNFDNCQNRERVFCLSIR |
| | | DDYLEKTGFKFKELEKVKNPPKKIKDILVDSSNYKYLNLNKYETTTF |
| | | RETKSNIISRSLKNYTTFNSENYVYNINGIGPTLTASGANSRIKIETQQ |
| | | GVRYLTPLECFKYMQFDVNDFKKVQSTNLISENKMIYIAGNSIPVKIL |
| | | EAIFNTLEFVNNEEGGPSSGAPPPSGGSPAGSPTSTEEGTSESATPESG |
| | | PGTSTEPSEGSAPGSPAGSPTSTEEGTSTEPSEGSAPGTSTEPSEPKKK |
| | | RKVMDKKYSIGLAIGTNSVGWAVITDEYKVPSKKFKVLGNTDRHSI |
| | | KKNLIGALLFDSGETAEATRLKRTARRRYTRRKNRICYLQEIFSNEM |
| | | AKVDDSFFHRLEESFLVEEDKKHERHPIFGNIVDEVAYHEKYPTIYHL |
| | | RKKLVDSTDKADLRLIYLALAHMIKFRGHFLIEGDLNPDNSDVDKLF |
| | | IQLVQTYNQLFEENPINASGVDAKAILSARLSKSRRLENLIAQLPGEK |
| | | KNGLFGNLIALSLGLTPNFKSNFDLAEDAKLQLSKDTYDDDLDNLLA |
| | | QIGDQYADLFLAAKNLSDAILLSDILRVNTEITKAPLSASMIKRYDEH |
| | | HQDLTLLKALVRQQLPEKYKEIFFDQSKNGYAGYIDGGASQEEFYKF |
| | | IKPILEKMDGTEELLVKLNREDLLRKQRTFDNGSIPHQIHLGELHAIL |
| | | RRQEDFYPFLKDNREKIEKILTFRIPYYVGPLARGNSRFAWMTRKSEE |
| | | TITPWNFEEVVDKGASAQSFIERMTNFDKNLPNEKVLPKHSLLYEYF |
| | | TVYNELTKVKYVTEGMRKPAFLSGEQKKAIVDLLFKTNRKVTVKQL |
| | | KEDYFKKIECFDSVEISGVEDRFNASLGTYHDLLKIIKDKDFLDNEEN |
| | | EDILEDIVLTLTLFEDREMIEERLKTYAHLFDDKVMKQLKRRRYTGW |
| | | GRLSRKLINGIRDKQSGKTILDFLKSDGFANRNFMQLIHDDSLTFKED |
| | | IQKAQVSGQGDSLHEHIANLAGSPAIKKGILQTVKVVDELVKVMGR |
| | | HKPENIVIEMARENQTTQKGQKNSRERMKRIEEGIKELGSQILKEHPV |
| | | ENTQLQNEKLYLYYLQNGRDMYVDQELDINRLSDYDVDAIVPQSFL |
| | | KDDSIDNKVLTRSDKNRGKSDNVPSEEVVKKMKNYWRQLLNAKLI |
| | | TQRKFDNLTKAERGGLSELDKAGFIKRQLVETRQITKHVAQILDSRM |
| | | NTKYDENDKLIREVKVITLKSKLVSDFRKDFQFYKVREINNYHHAHD |
| | | AYLNAVVGTALIKKYPKLESEFVYGDYKVYDVRKMIAKSEQEIGKA |
| | | TAKYFFYSNIMNFFKTEITLANGEIRKRPLIETNGETGEIVWDKGRDF |
| | | ATVRKVLSMPQVNIVKKTEVQTGGFSKESILPKRNSDKLIARKKDW |
| | | DPKKYGGFDSPTVAYSVLVVAKVEKGKSKKLKSVKELLGITIMERSS |
| | | FEKNPIDFLEAKGYKEVKKDLIIKLPKYSLFELENGRKRMLASAGEL |
| | | QKGNELALPSKYVNFLYLASHYEKLKGSPEDNEQKQLFVEQHKHYL |
| | | DEIIEQISEFSKRVILADANLDKVLSAYNKHRDKPIREQAENIIHLFTLT |
| | | NLGAPAAFKYFDTTIDRKRYTSTKEVLDATLIHQSITGLYETRIDLSQ |
| | | LGGDPKKKRKV |
| 1125 | (Sm)M.SssI- | MSKVENKTKKLRVFEAFAGIGAQRKALEKVRKDEYEIVGLAEWYVP |
| | Cas | AIVMYQAIHNNFHTKLEYKSVSREEMIDYLENKTLSWNSKNPVSNG |
| | | YWKRKKDDELKIIYNAIKLSEKEGNIFDIRDLYKRTLKNIDLLTYSFP |
| | | CQDLSQQGIQKGMKRGSGTRSGLLWEIERALDSTEKNDLPKYLLME |
| | | NVGALLHKKNEEELNQWKQKLESLGYQNSIEVLNAADFGSSQARRR |
| | | VFMISTLNEFVELPKGDKKPKSIKKVLNKIVSEKDILNNLLKYNLTEF |
| | | KKTKSNINKASLIGYSKFNSEGYVYDPEFTGPTLTASGANSRIKIKDG |
| | | SNIRKMNSDETFLYIGFDSQDGKRVNEIEFLTENQKIFVCGNSISVEVL |
| | | EAIIDKIGGGGPSSGAPPPSGGSPAGSPTSTEEGTSESATPESGPGTSTE |
| | | PSEGSAPGSPAGSPTSTEEGTSTEPSEGSAPGTSTEPSEPKKKRKVMD |
| | | KKYSIGLAIGTNSVGWAVITDEYKVPSKKFKVLGNTDRHSIKKNLIG |
| | | ALLFDSGETAEATRLKRTARRRYTRRKNRICYLQEIFSNEMAKVDDS |
| | | FFHRLEESFLVEEDKKHERHPIFGNIVDEVAYHEKYPTIYHLRKKLVD |
| | | STDKADLRLIYLALAHMIKFRGHFLIEGDLNPDNSDVDKLFIQLVQT |
| | | YNQLFEENPINASGVDAKAILSARLSKSRRLENLIAQLPGEKKNGLFG |
| | | NLIALSLGLTPNFKSNFDLAEDAKLQLSKDTYDDDLDNLLAQIGDQY |
| | | ADLFLAAKNLSDAILLSDILRVNTEITKAPLSASMIKRYDEHHQDLTL |
| | | LKALVRQQLPEKYKEIFFDQSKNGYAGYIDGGASQEEFYKFIKPILEK |
| | | MDGTEELLVKLNREDLLRKQRTFDNGSIPHQIHLGELHAILRRQEDF |
| | | YPFLKDNREKIEKILTFRIPYYVGPLARGNSRFAWMTRKSEETITPWN |
| | | FEEVVDKGASAQSFIERMTNFDKNLPNEKVLPKHSLLYEYFTVYNEL |
| | | TKVKYVTEGMRKPAFLSGEQKKAIVDLLFKTNRKVTVKQLKEDYFK |
| | | KIECFDSVEISGVEDRFNASLGTYHDLLKIIKDKDFLDNEENEDILEDI |
| | | VLTLTLFEDREMIEERLKTYAHLFDDKVMKQLKRRRYTGWGRLSRK |
| | | LINGIRDKQSGKTILDFLKSDGFANRNFMQLIHDDSLTFKEDIQKAQV |
| | | SGQGDSLHEHIANLAGSPAIKKGILQTVKVVDELVKVMGRHKPENIV |
| | | IEMARENQTTQKGQKNSRERMKRIEEGIKELGSQILKEHPVENTQLQ |
| | | NEKLYLYYLQNGRDMYVDQELDINRLSDYDVDAIVPQSFLKDDSID |
| | | NKVLTRSDKNRGKSDNVPSEEVVKKMKNYWRQLLNAKLITQRKFD |
| | | NLTKAERGGLSELDKAGFIKRQLVETRQITKHVAQILDSRMNTKYDE |
| | | NDKLIREVKVITLKSKLVSDFRKDFQFYKVREINNYHHAHDAYLNA |
| | | VVGTALIKKYPKLESEFVYGDYKVYDVRKMIAKSEQEIGKATAKYF |
| | | FYSNIMNFFKTEITLANGEIRKRPLIETNGETGEIVWDKGRDFATVRK |
| | | VLSMPQVNIVKKTEVQTGGFSKESILPKRNSDKLIARKKDWDPKKY |
| | | GGFDSPTVAYSVLVVAKVEKGKSKKLKSVKELLGITIMERSSFEKNPI |
| | | DFLEAKGYKEVKKDLIIKLPKYSLFELENGRKRMLASAGELQKGNEL |
| | | ALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPA |
| | | AFKYFDTTIDRKRYTSTKEVLDATLIHQSITGLYETRIDLSQLGGDPK |
| | | KKRKV |
| 1126 | (Hp)M.HpaII- | MKDVLDDNLLEEPAAQYSLFEPESNPNLREKFTFIDLFAGIGGFRIAM |
| | Cas | QNLGGKCIFSSEWDEQAQKTYEANFGDLPYGDITLEETKAFIPEKFDI |
| | | LCAGFPCQAFSIAGKRGGFEDTRGTLFFDVAEIIRRHQPKAFFLENVK |
| | | GLKNHDKGRTLKTILNVLREDLGYFVPEPAIVNAKNFGVPQNRERIY |
| | | IVGFHKSTGVNSFSYPEPLDKIVTFADIREEKTVPTKYYLSTQYIDTLR |
| | | KHKERHESKGNGFGYEIIPDDGIANAIVVGGMGRERNLVIDHRITDFT |
| | | PTTNIKGEVNREGIRKMTPREWARLQGFPDSYVIPVSDASAYKQFGN |
| | | SVAVPAIQATGKKILEKLGNLYDGGPSSGAPPPSGGSPAGSPTSTEEG |
| | | TSESATPESGPGTSTEPSEGSAPGSPAGSPTSTEEGTSTEPSEGSAPGTS |
| | | TEPSEPKKKRKVMDKKYSIGLAIGTNSVGWAVITDEYKVPSKKFKVL |
| | | GNTDRHSIKKNLIGALLFDSGETAEATRLKRTARRRYTRRKNRICYL |
| | | QEIFSNEMAKVDDSFFHRLEESFLVEEDKKHERHPIFGNIVDEVAYHE |
| | | KYPTIYHLRKKLVDSTDKADLRLIYLALAHMIKFRGHFLIEGDLNPD |
| | | NSDVDKLFIQLVQTYNQLFEENPINASGVDAKAILSARLSKSRRLENL |
| | | IAQLPGEKKNGLFGNLIALSLGLTPNFKSNFDLAEDAKLQLSKDTYD |
| | | DDLDNLLAQIGDQYADLFLAAKNLSDAILLSDILRVNTEITKAPLSAS |
| | | MIKRYDEHHQDLTLLKALVRQQLPEKYKEIFFDQSKNGYAGYIDGG |
| | | ASQEEFYKFIKPILEKMDGTEELLVKLNREDLLRKQRTFDNGSIPHQI |
| | | HLGELHAILRRQEDFYPFLKDNREKIEKILTFRIPYYVGPLARGNSRF |
| | | AWMTRKSEETITPWNFEEVVDKGASAQSFIERMTNFDKNLPNEKVL |
| | | PKHSLLYEYFTVYNELTKVKYVTEGMRKPAFLSGEQKKAIVDLLFK |
| | | TNRKVTVKQLKEDYFKKIECFDSVEISGVEDRFNASLGTYHDLLKIIK |
| | | DKDFLDNEENEDILEDIVLTLTLFEDREMIEERLKTYAHLFDDKVMK |
| | | QLKRRRYTGWGRLSRKLINGIRDKQSGKTILDFLKSDGFANRNFMQL |
| | | IHDDSLTFKEDIQKAQVSGQGDSLHEHIANLAGSPAIKKGILQTVKVV |
| | | DELVKVMGRHKPENIVIEMARENQTTQKGQKNSRERMKRIEEGIKE |
| | | LGSQILKEHPVENTQLQNEKLYLYYLQNGRDMYVDQELDINRLSDY |
| | | DVDAIVPQSFLKDDSIDNKVLTRSDKNRGKSDNVPSEEVVKKMKNY |
| | | WRQLLNAKLITQRKFDNLTKAERGGLSELDKAGFIKRQLVETRQITK |
| | | HVAQILDSRMNTKYDENDKLIREVKVITLKSKLVSDFRKDFQFYKVR |
| | | EINNYHHAHDAYLNAVVGTALIKKYPKLESEFVYGDYKVYDVRKMI |
| | | AKSEQEIGKATAKYFFYSNIMNFFKTEITLANGEIRKRPLIETNGETGE |
| | | IVWDKGRDFATVRKVLSMPQVNIVKKTEVQTGGFSKESILPKRNSDK |
| | | LIARKKDWDPKKYGGFDSPTVAYSVLVVAKVEKGKSKKLKSVKEL |
| | | LGITIMERSSFEKNPIDFLEAKGYKEVKKDLIIKLPKYSLFELENGRKR |
| | | MLASAGELQKGNELALPSKYVNFLYLASHYEKLKGSPEDNEQKQLF |
| | | VEQHKHYLDEIIEQISEFSKRVILADANLDKVLSAYNKHRDKPIREQA |
| | | ENIIHLFTLTNLGAPAAFKYFDTTIDRKRYTSTKEVLDATLIHQSITGL |
| | | YETRIDLSQLGGDPKKKRKV |
| 1127 | (Al)M.AluI- | MSKANAKYSFVDLFAGIGGFHAALAATGGVCEYAVEIDREAAAVYE |
| | Cas | RNWNKPALGDITDDANDEGVTLRGYDGPIDVLTGGFPCQPFSKSGA |
| | | QHGMAETRGTLFWNIARIIEEREPTVLILENVRNLVGPRHRHEWLTII |
| | | ETLRFFGYEVSGAPAIFSPHLLPAWMGGTPQVRERVFITATLVPERM |
| | | RDERIPRTETGEIDAEAIGPKPVATMNDRFPIKKGGTELFHPGDRKSG |
| | | WNLLTSGIIREGDPEPSNVDLRLTETETLWIDAWDDLESTIRRATGRP |
| | | LEGFPYWADSWTDFRELSRLVVIRGFQAPEREVVGDRKRYVARTDM |
| | | PEGFVPASVTRPAIDETLPAWKQSHLRRNYDFFERHFAEVVAWAYR |
| | | WGVYTDLFPASRRKLEWQAQDAPRLWDTVMHFRPSGIRAKRPTYL |
| | | PALVAITQTSIVGPLERRLSPRETARLQGLPEWFDFGEQRAAATYKQ |
| | | MGNGVNVGVVRHILREHVRRDRALLKLTPAGQRIINAVLADEPDAT |
| | | VGALGAAEGGPSSGAPPPSGGSPAGSPTSTEEGTSESATPESGPGTST |
| | | EPSEGSAPGSPAGSPTSTEEGTSTEPSEGSAPGTSTEPSEPKKKRKVM |
| | | DKKYSIGLAIGTNSVGWAVITDEYKVPSKKFKVLGNTDRHSIKKNLI |
| | | GALLFDSGETAEATRLKRTARRRYTRRKNRICYLQEIFSNEMAKVDD |
| | | SFFHRLEESFLVEEDKKHERHPIFGNIVDEVAYHEKYPTIYHLRKKLV |
| | | DSTDKADLRLIYLALAHMIKFRGHFLIEGDLNPDNSDVDKLFIQLVQ |
| | | TYNQLFEENPINASGVDAKAILSARLSKSRRLENLIAQLPGEKKNGLF |
| | | GNLIALSLGLTPNFKSNFDLAEDAKLQLSKDTYDDDLDNLLAQIGDQ |
| | | YADLFLAAKNLSDAILLSDILRVNTEITKAPLSASMIKRYDEHHQDLT |
| | | LLKALVRQQLPEKYKEIFFDQSKNGYAGYIDGGASQEEFYKFIKPILE |
| | | KMDGTEELLVKLNREDLLRKQRTFDNGSIPHQIHLGELHAILRRQED |
| | | FYPFLKDNREKIEKILTFRIPYYVGPLARGNSRFAWMTRKSEETITPW |
| | | NFEEVVDKGASAQSFIERMTNFDKNLPNEKVLPKHSLLYEYFTVYNE |
| | | LTKVKYVTEGMRKPAFLSGEQKKAIVDLLFKTNRKVTVKQLKEDYF |
| | | KKIECFDSVEISGVEDRFNASLGTYHDLLKIIKDKDFLDNEENEDILED |
| | | IVLTLTLFEDREMIEERLKTYAHLFDDKVMKQLKRRRYTGWGRLSR |
| | | KLINGIRDKQSGKTILDFLKSDGFANRNFMQLIHDDSLTFKEDIQKAQ |
| | | VSGQGDSLHEHIANLAGSPAIKKGILQTVKVVDELVKVMGRHKPENI |
| | | VIEMARENQTTQKGQKNSRERMKRIEEGIKELGSQILKEHPVENTQL |
| | | QNEKLYLYYLQNGRDMYVDQELDINRLSDYDVDAIVPQSFLKDDSI |
| | | DNKVLTRSDKNRGKSDNVPSEEVVKKMKNYWRQLLNAKLITQRKF |
| | | DNLTKAERGGLSELDKAGFIKRQLVETRQITKHVAQILDSRMNTKYD |
| | | ENDKLIREVKVITLKSKLVSDFRKDFQFYKVREINNYHHAHDAYLNA |
| | | VVGTALIKKYPKLESEFVYGDYKVYDVRKMIAKSEQEIGKATAKYF |
| | | FYSNIMNFFKTEITLANGEIRKRPLIETNGETGEIVWDKGRDFATVRK |
| | | VLSMPQVNIVKKTEVQTGGFSKESILPKRNSDKLIARKKDWDPKKY |
| | | GGFDSPTVAYSVLVVAKVEKGKSKKLKSVKELLGITIMERSSFEKNPI |
| | | DFLEAKGYKEVKKDLIIKLPKYSLFELENGRKRMLASAGELQKGNEL |
| | | ALPSKYVNFLYLASHYEKLKGSPEDNEQKQLFVEQHKHYLDEIIEQIS |
| | | EFSKRVILADANLDKVLSAYNKHRDKPIREQAENIIHLFTLTNLGAPA |
| | | AFKYFDTTIDRKRYTSTKEVLDATLIHQSITGLYETRIDLSQLGGDPK |
| | | KKRKV |
| 1128 | (Al)M.AluI- | MSKANAKYSFVDLFAGIGGFHAALAATGGVCEYAVEIDREAAAVYE |
| | de182-Cas | RNWNKPALGDITDDANDEGVTLRGYDGPIDVLTGGFPCQPFSKSGA |
| | | QHGMAETRGTLFWNIARIIEEREPTVLILENVRNLVGPRHRHEWLTII |
| | | ETLRFFGYEVSGAPAIFSPHLLPAWMGGTPQVRERVFITATLVPERM |
| | | RDERSTIRRATGRPLEGFPYWADSWTDFRELSRLVVIRGFQAPEREV |
| | | VGDRKRYVARTDMPEGFVPASVTRPAIDETLPAWKQSHLRRNYDFF |
| | | ERHFAEVVAWAYRWGVYTDLFPASRRKLEWQAQDAPRLWDTVMH |
| | | FRPSGIRAKRPTYLPALVAITQTSIVGPLERRLSPRETARLQGLPEWFD |
| | | FGEQRAAATYKQMGNGVNVGVVRHILREHVRRDRALLKLTPAGQR |
| | | IINAVLADEPDATVGALGAAEGGPSSGAPPPSGGSPAGSPTSTEEGTS |
| | | ESATPESGPGTSTEPSEGSAPGSPAGSPTSTEEGTSTEPSEGSAPGTSTE |
| | | PSEPKKKRKVMDKKYSIGLAIGTNSVGWAVITDEYKVPSKKFKVLG |
| | | NTDRHSIKKNLIGALLFDSGETAEATRLKRTARRRYTRRKNRICYLQ |
| | | EIFSNEMAKVDDSFFHRLEESFLVEEDKKHERHPIFGNIVDEVAYHEK |
| | | YPTIYHLRKKLVDSTDKADLRLIYLALAHMIKFRGHFLIEGDLNPDNS |
| | | DVDKLFIQLVQTYNQLFEENPINASGVDAKAILSARLSKSRRLENLIA |
| | | QLPGEKKNGLFGNLIALSLGLTPNFKSNFDLAEDAKLQLSKDTYDDD |
| | | LDNLLAQIGDQYADLFLAAKNLSDAILLSDILRVNTEITKAPLSASMI |
| | | KRYDEHHQDLTLLKALVRQQLPEKYKEIFFDQSKNGYAGYIDGGAS |
| | | QEEFYKFIKPILEKMDGTEELLVKLNREDLLRKQRTFDNGSIPHQIHL |
| | | GELHAILRRQEDFYPFLKDNREKIEKILTFRIPYYVGPLARGNSRFAW |
| | | MTRKSEETITPWNFEEVVDKGASAQSFIERMTNFDKNLPNEKVLPKH |
| | | SLLYEYFTVYNELTKVKYVTEGMRKPAFLSGEQKKAIVDLLFKTNR |
| | | KVTVKQLKEDYFKKIECFDSVEISGVEDRFNASLGTYHDLLKIIKDKD |
| | | FLDNEENEDILEDIVLTLTLFEDREMIEERLKTYAHLFDDKVMKQLK |
| | | RRRYTGWGRLSRKLINGIRDKQSGKTILDFLKSDGFANRNFMQLIHD |
| | | DSLTFKEDIQKAQVSGQGDSLHEHIANLAGSPAIKKGILQTVKVVDE |
| | | LVKVMGRHKPENIVIEMARENQTTQKGQKNSRERMKRIEEGIKELGS |
| | | QILKEHPVENTQLQNEKLYLYYLQNGRDMYVDQELDINRLSDYDVD |
| | | AIVPQSFLKDDSIDNKVLTRSDKNRGKSDNVPSEEVVKKMKNYWRQ |
| | | LLNAKLITQRKFDNLTKAERGGLSELDKAGFIKRQLVETRQITKHVA |
| | | QILDSRMNTKYDENDKLIREVKVITLKSKLVSDFRKDFQFYKVREIN |
| | | NYHHAHDAYLNAVVGTALIKKYPKLESEFVYGDYKVYDVRKMIAK |
| | | SEQEIGKATAKYFFYSNIMNFFKTEITLANGEIRKRPLIETNGETGEIV |
| | | WDKGRDFATVRKVLSMPQVNIVKKTEVQTGGFSKESILPKRNSDKLI |
| | | ARKKDWDPKKYGGFDSPTVAYSVLVVAKVEKGKSKKLKSVKELLG |
| | | ITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKV |
| 1129 | (Ha) | MNLISLFSGAGGLDLGFQKAGFRIICANEYDKSIWKTYESNHSAKLIK |
| | M.HaeIII- | GDISKISSDEFPKCDGIIGGPPCQSWSEGGSLRGIDDPRGKLFYEYIRIL |
| | Cas | KQKKPIFFLAENVKGMMAQRHNKAVQEFIQEFDNAGYDVHIILLNA |
| | | NDYGVAQDRKRVFYIGFRKELNINYLPPIPHLIKPTFKDVIWDLKDNP |
| | | IPALDKNKTNGNKCIYPNHEYFIGSYSTIFMSRNRVRQWNEPAFTVQ |
| | | ASGRQCQLHPQAPVMLKVSKNLNKFVEGKEHLYRRLTVRECARVQ |
| | | GFPDDFIFHYESLNDGYKMIGNAVPVNLAYEIAKTIKSALEICKGNGG |
| | | PSSGAPPPSGGSPAGSPTSTEEGTSESATPESGPGTSTEPSEGSAPGSPA |
| | | GSPTSTEEGTSTEPSEGSAPGTSTEPSEPKKKRKVMDKKYSIGLAIGT |
| | | NSVGWAVITDEYKVPSKKFKVLGNTDRHSIKKNLIGALLFDSGETAE |
| | | ATRLKRTARRRYTRRKNRICYLQEIFSNEMAKVDDSFFHRLEESFLV |
| | | EEDKKHERHPIFGNIVDEVAYHEKYPTIYHLRKKLVDSTDKADLRLI |
| | | YLALAHMIKFRGHFLIEGDLNPDNSDVDKLFIQLVQTYNQLFEENPIN |
| | | ASGVDAKAILSARLSKSRRLENLIAQLPGEKKNGLFGNLIALSLGLTP |
| | | NFKSNFDLAEDAKLQLSKDTYDDDLDNLLAQIGDQYADLFLAAKNL |
| | | SDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVK |
| | | LNREDLLRKQRTFDNGSIPHQIHLGELHAILRRQEDFYPFLKDNREKI |
| | | EKILTFRIPYYVGPLARGNSRFAWMTRKSEETITPWNFEEVVDKGAS |
| | | AQSFIERMTNFDKNLPNEKVLPKHSLLYEYFTVYNELTKVKYVTEG |
| | | MRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFED |
| | | REMIEERLKTYAHLFDDKVMKQLKRRRYTGWGRLSRKLINGIRDKQ |
| | | SGKTILDFLKSDGFANRNFMQLIHDDSLTFKEDIQKAQVSGQGDSLH |
| | | EHIANLAGSPAIKKGILQTVKVVDELVKVMGRHKPENIVIEMARENQ |
| | | TTQKGQKNSRERMKRIEEGIKELGSQILKEHPVENTQLQNEKLYLYY |
| | | LQNGRDMYVDQELDINRLSDYDVDAIVPQSFLKDDSIDNKVLTRSD |
| | | KNRGKSDNVPSEEVVKKMKNYWRQLLNAKLITQRKFDNLTKAERG |
| | | GLSELDKAGFIKRQLVETRQITKHVAQILDSRMNTKYDENDKLIREV |
| | | KVITLKSKLVSDFRKDFQFYKVREINNYHHAHDAYLNAVVGTALIK |
| | | KYPKLESEFVYGDYKVYDVRKMIAKSEQEIGKATAKYFFYSNIMNFF |
| | | KTEITLANGEIRKRPLIETNGETGEIVWDKGRDFATVRKVLSMPQVNI |
| | | VKKTEVQTGGFSKESILPKRNSDKLIARKKDWDPKKYGGFDSPTVA |
| | | YSVLVVAKVEKGKSKKLKSVKELLGITIMERSSFEKNPIDFLEAKGY |
| | | KEVKKDLIIKLPKYSLFELENGRKRMLASAGELQKGNELALPSKYVN |
| | | FLYLASHYEKLKGSPEDNEQKQLFVEQHKHYLDEIIEQISEFSKRVIL |
| | | ADANLDKVLSAYNKHRDKPIREQAENIIHLFTLTNLGAPAAFKYFDT |
| | | TIDRKRYTSTKEVLDATLIHQSITGLYETRIDLSQLGGDPKKKRKV |
| 1130 | (Ha) | MNLISLFSGAGGLDLGFQKAGFRIICANEYDKSIWKTYESNHSAKLIK |
| | M.HaeIII- | GDISKISSDEFPKCDGIIGGPPCQSWSEGGSLRGIDDPRGKLFYEYIRIL |
| | T29-Cas | KQKKPIFFLAENVKGMMAQRHNKAVQEFIQEFDNAGYDVHIILLNA |
| | | NDYGVAQDRKRVFYIGFRKELNINYLPPIPHLIKPTFKDVIWDLKDNP |
| | | IPALDKNKTNGNKCIYPNHEYFIGSYSTIFMSANRVRQWNEPAFTVQ |
| | | ASGRQCQLHPQAPVMLKVSKLMWKFVEGKEHLYRRLTVRECARVQ |
| | | GFPDDFIFHYESLNDGYKMIGNAVPVNLAYEIAKTIKSALEICKGNGG |
| | | PSSGAPPPSGGSPAGSPTSTEEGTSESATPESGPGTSTEPSEGSAPGSPA |
| | | GSPTSTEEGTSTEPSEGSAPGTSTEPSEPKKKRKVMDKKYSIGLAIGT |
| | | NSVGWAVITDEYKVPSKKFKVLGNTDRHSIKKNLIGALLFDSGETAE |
| | | ATRLKRTARRRYTRRKNRICYLQEIFSNEMAKVDDSFFHRLEESFLV |
| | | EEDKKHERHPIFGNIVDEVAYHEKYPTIYHLRKKLVDSTDKADLRLI |
| | | YLALAHMIKFRGHFLIEGDLNPDNSDVDKLFIQLVQTYNQLFEENPIN |
| | | ASGVDAKAILSARLSKSRRLENLIAQLPGEKKNGLFGNLIALSLGLTP |
| | | NFKSNFDLAEDAKLQLSKDTYDDDLDNLLAQIGDQYADLFLAAKNL |
| | | SDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVK |
| | | LNREDLLRKQRTFDNGSIPHQIHLGELHAILRRQEDFYPFLKDNREKI |
| | | EKILTFRIPYYVGPLARGNSRFAWMTRKSEETITPWNFEEVVDKGAS |
| | | AQSFIERMTNFDKNLPNEKVLPKHSLLYEYFTVYNELTKVKYVTEG |
| | | MRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFED |
| | | REMIEERLKTYAHLFDDKVMKQLKRRRYTGWGRLSRKLINGIRDKQ |
| | | SGKTILDFLKSDGFANRNFMQLIHDDSLTFKEDIQKAQVSGQGDSLH |
| | | EHIANLAGSPAIKKGILQTVKVVDELVKVMGRHKPENIVIEMARENQ |
| | | TTQKGQKNSRERMKRIEEGIKELGSQILKEHPVENTQLQNEKLYLYY |
| | | LQNGRDMYVDQELDINRLSDYDVDAIVPQSFLKDDSIDNKVLTRSD |
| | | KNRGKSDNVPSEEVVKKMKNYWRQLLNAKLITQRKFDNLTKAERG |
| | | GLSELDKAGFIKRQLVETRQITKHVAQILDSRMNTKYDENDKLIREV |
| | | KVITLKSKLVSDFRKDFQFYKVREINNYHHAHDAYLNAVVGTALIK |
| | | KYPKLESEFVYGDYKVYDVRKMIAKSEQEIGKATAKYFFYSNIMNFF |
| | | KTEITLANGEIRKRPLIETNGETGEIVWDKGRDFATVRKVLSMPQVNI |
| | | VKKTEVQTGGFSKESILPKRNSDKLIARKKDWDPKKYGGFDSPTVA |
| | | YSVLVVAKVEKGKSKKLKSVKELLGITIMERSSFEKNPIDFLEAKGY |
| | | KEVKKDLIIKLPKYSLFELENGRKRMLASAGELQKGNELALPSKYVN |
| | | FLYLASHYEKLKGSPEDNEQKQLFVEQHKHYLDEIIEQISEFSKRVIL |
| | | ADANLDKVLSAYNKHRDKPIREQAENIIHLFTLTNLGAPAAFKYFDT |
| | | TIDRKRYTSTKEVLDATLIHQSITGLYETRIDLSQLGGDPKKKRKV |
| 1131 | (Hh)M.HhaI- | MIEIKDKQLTGLRFIDLFAGLGGFRLALESCGAECVYSNEWDKYAQE |
| | Cas | VYEMNFGEKPEGDITQVNEKTIPDHDILCAGFPCQAFSISGKQKGFED |
| | | SRGTLFFDIARIVREKKPKVVFMENVKNFASHDNGNTLEVVKNTMN |
| | | ELDYSFHAKVLNALDYGIPQKRERIYMICFRNDLNIQNFQFPKPFELN |
| | | TFVKDLLLPDSEVEHLVIDRKDLVMTNQEIEQTTPKTVRLGIVGKGG |
| | | QGERIYSTRGIAITLSAYGGGIFAKTGGYLVNGKTRKLHPRECARVM |
| | | GYPDSYKVHPSTSQAYKQFGNSVVINVLQYIAYNIGSSLNFKPYGGP |
| | | SSGAPPPSGGSPAGSPTSTEEGTSESATPESGPGTSTEPSEGSAPGSPA |
| | | GSPTSTEEGTSTEPSEGSAPGTSTEPSEPKKKRKVMDKKYSIGLAIGT |
| | | NSVGWAVITDEYKVPSKKFKVLGNTDRHSIKKNLIGALLFDSGETAE |
| | | ATRLKRTARRRYTRRKNRICYLQEIFSNEMAKVDDSFFHRLEESFLV |
| | | EEDKKHERHPIFGNIVDEVAYHEKYPTIYHLRKKLVDSTDKADLRLI |
| | | YLALAHMIKFRGHFLIEGDLNPDNSDVDKLFIQLVQTYNQLFEENPIN |
| | | ASGVDAKAILSARLSKSRRLENLIAQLPGEKKNGLFGNLIALSLGLTP |
| | | NFKSNFDLAEDAKLQLSKDTYDDDLDNLLAQIGDQYADLFLAAKNL |
| | | SDAILLSDILRVNTEITKAPLSASMIKRYDEHHQDLTLLKALVRQQLP |
| | | EKYKEIFFDQSKNGYAGYIDGGASQEEFYKFIKPILEKMDGTEELLVK |
| | | LNREDLLRKQRTFDNGSIPHQIHLGELHAILRRQEDFYPFLKDNREKI |
| | | EKILTFRIPYYVGPLARGNSRFAWMTRKSEETITPWNFEEVVDKGAS |
| | | AQSFIERMTNFDKNLPNEKVLPKHSLLYEYFTVYNELTKVKYVTEG |
| | | MRKPAFLSGEQKKAIVDLLFKTNRKVTVKQLKEDYFKKIECFDSVEI |
| | | SGVEDRFNASLGTYHDLLKIIKDKDFLDNEENEDILEDIVLTLTLFED |
| | | REMIEERLKTYAHLFDDKVMKQLKRRRYTGWGRLSRKLINGIRDKQ |
| | | SGKTILDFLKSDGFANRNFMQLIHDDSLTFKEDIQKAQVSGQGDSLH |
| | | EHIANLAGSPAIKKGILQTVKVVDELVKVMGRHKPENIVIEMARENQ |
| | | TTQKGQKNSRERMKRIEEGIKELGSQILKEHPVENTQLQNEKLYLYY |
| | | LQNGRDMYVDQELDINRLSDYDVDAIVPQSFLKDDSIDNKVLTRSD |
| | | KNRGKSDNVPSEEVVKKMKNYWRQLLNAKLITQRKFDNLTKAERG |
| | | GLSELDKAGFIKRQLVETRQITKHVAQILDSRMNTKYDENDKLIREV |
| | | KVITLKSKLVSDFRKDFQFYKVREINNYHHAHDAYLNAVVGTALIK |
| | | KYPKLESEFVYGDYKVYDVRKMIAKSEQEIGKATAKYFFYSNIMNFF |
| | | KTEITLANGEIRKRPLIETNGETGEIVWDKGRDFATVRKVLSMPQVNI |
| | | VKKTEVQTGGFSKESILPKRNSDKLIARKKDWDPKKYGGFDSPTVA |
| | | YSVLVVAKVEKGKSKKLKSVKELLGITIMERSSFEKNPIDFLEAKGY |
| | | KEVKKDLIIKLPKYSLFELENGRKRMLASAGELQKGNELALPSKYVN |
| | | FLYLASHYEKLKGSPEDNEQKQLFVEQHKHYLDEIIEQISEFSKRVIL |
| | | ADANLDKVLSAYNKHRDKPIREQAENIIHLFTLTNLGAPAAFKYFDT |
| | | TIDRKRYTSTKEVLDATLIHQSITGLYETRIDLSQLGGDPKKKRKV |
| 1132 | (Ms)M.MspI- | MKPEILKLIRSKLDLTQKQASEIIEVSDKTWQQWESGKTEMHPAYYS |
| | Cas | FLQEKLKDKINFEELSAQKTLQKKIFDKYNQNQITKNAEELAEITHIE |
| | | ERKDAYSSDFKFIDLFSGIGGIRQSFEVNGGKCVFSSEIDPFAKFTYYT |
| | | NFGVVPFGDITKVEATTIPQHDILCAGFPCQPFSHIGKREGFEHPTQGT |
| | | MFHEIVRIIETKKTPVLFLENVPGLINHDDGNTLKVIIETLEDMGYKV |
| | | HHTVLDASHFGIPQKRKRFYLVAFLNQNIHFEFPKPPMISKDIGEVLE |
| | | SDVTGYSISEHLQKSYLFKKDDGKPSLIDKNTTGAVKTLVSTYHKIQ |
| | | RLTGTFVKDGETGIRLLTTNECKAIMGFPKDFVIPVSRTQMYRQMGN |
| | | SVVVPVVTKIAEQISLALKTVNQQSPQENFELELVGGPSSGAPPPSGG |
| | | SPAGSPTSTEEGTSESATPESGPGTSTEPSEGSAPGSPAGSPTSTEEGTS |
| | | TEPSEGSAPGTSTEPSEPKKKRKVMDKKYSIGLAIGTNSVGWAVITD |
| | | EYKVPSKKFKVLGNTDRHSIKKNLIGALLFDSGETAEATRLKRTARR |
| | | RYTRRKNRICYLQEIFSNEMAKVDDSFFHRLEESFLVEEDKKHERHPI |
| | | FGNIVDEVAYHEKYPTIYHLRKKLVDSTDKADLRLIYLALAHMIKFR |
| | | GHFLIEGDLNPDNSDVDKLFIQLVQTYNQLFEENPINASGVDAKAILS |
| | | ARLSKSRRLENLIAQLPGEKKNGLFGNLIALSLGLTPNFKSNFDLAED |
| | | AKLQLSKDTYDDDLDNLLAQIGDQYADLFLAAKNLSDAILLSDILRV |
| | | NTEITKAPLSASMIKRYDEHHQDLTLLKALVRQQLPEKYKEIFFDQS |
| | | KNGYAGYIDGGASQEEFYKFIKPILEKMDGTEELLVKLNREDLLRKQ |
| | | RTFDNGSIPHQIHLGELHAILRRQEDFYPFLKDNREKIEKILTFRIPYY |
| | | VGPLARGNSRFAWMTRKSEETITPWNFEEVVDKGASAQSFIERMTN |
| | | FDKNLPNEKVLPKHSLLYEYFTVYNELTKVKYVTEGMRKPAFLSGE |
| | | QKKAIVDLLFKTNRKVTVKQLKEDYFKKIECFDSVEISGVEDRFNAS |
| | | LGTYHDLLKIIKDKDFLDNEENEDILEDIVLTLTLFEDREMIEERLKTY |
| | | AHLFDDKVMKQLKRRRYTGWGRLSRKLINGIRDKQSGKTILDFLKS |
| | | DGFANRNFMQLIHDDSLTFKEDIQKAQVSGQGDSLHEHIANLAGSPA |
| | | IKKGILQTVKVVDELVKVMGRHKPENIVIEMARENQTTQKGQKNSR |
| | | ERMKRIEEGIKELGSQILKEHPVENTQLQNEKLYLYYLQNGRDMYV |
| | | DQELDINRLSDYDVDAIVPQSFLKDDSIDNKVLTRSDKNRGKSDNVP |
| | | SEEVVKKMKNYWRQLLNAKLITQRKFDNLTKAERGGLSELDKAGFI |
| | | KRQLVETRQITKHVAQILDSRMNTKYDENDKLIREVKVITLKSKLVS |
| | | DFRKDFQFYKVREINNYHHAHDAYLNAVVGTALIKKYPKLESEFVY |
| | | GDYKVYDVRKMIAKSEQEIGKATAKYFFYSNIMNFFKTEITLANGEI |
| | | RKRPLIETNGETGEIVWDKGRDFATVRKVLSMPQVNIVKKTEVQTG |
| | | GFSKESILPKRNSDKLIARKKDWDPKKYGGFDSPTVAYSVLVVAKV |
| | | EKGKSKKLKSVKELLGITIMERSSFEKNPIDFLEAKGYKEVKKDLIIK |
| | | LPKYSLFELENGRKRMLASAGELQKGNELALPSKYVNFLYLASHYE |
| | | KLKGSPEDNEQKQLFVEQHKHYLDEIIEQISEFSKRVILADANLDKVL |
| | | SAYNKHRDKPIREQAENIIHLFTLTNLGAPAAFKYFDTTIDRKRYTST |
| | | KEVLDATLIHQSITGLYETRIDLSQLGGDPKKKRKV |
| 1133 | (Ai)Masc1- | MSERRYEAGMTVALHEGSFLKIQRVYIRQYHADNRREHMLVGPLFR |
| | Cas | RTKYLKALSKKVNEVAIVHESIHVPVQDVIGVRELIITNRPFPECRKG |
| | | DEHTGRLVCRWVYNLDERAKGREYKKQRYIRRITEAEADPEYRVED |
| | | RVLRRRWFQEGYIGDEISYKEHGNGDIVDIRSESPLQVLDGWGGDLV |
| | | DLENGEETSIPGPCRSASSYGRLMKPPLAQAADSNTSRKYTFGDTFC |
| | | GGGGVSLGARQAGLEVKWAFDMNPNAGANYRRNFPNTDFFLAEAE |
| | | QFIQLSVGISQHVDILHLSPPCQTFSRAHTIAGKNDENNEASFFAVVN |
| | | LIKAVRPRLFTVEETDGIMDRQSRQFIDTALMGITELGYSFRICVLNAI |
| | | EYGVCQNRKRLIIIGAAPGEELPPFPLPTHQDFFSKDPRRDLLPAVTLD |
| | | DALSTITPESTDHHLNHVWQPAEWKTPYDAHRPFKNAIRAGGGEYD |
| | | IYPDGRRKFTVRELACIQGFPDEYEFVGTLTDKRRIIGNAVPPPLSAAI |
| | | MSTLRQWMTEKDFERMEGGPSSGAPPPSGGSPAGSPTSTEEGTSESA |
| | | TPESGPGTSTEPSEGSAPGSPAGSPTSTEEGTSTEPSEGSAPGTSTEPSE |
| | | PKKKRKVMDKKYSIGLAIGTNSVGWAVITDEYKVPSKKFKVLGNTD |
| | | RHSIKKNLIGALLFDSGETAEATRLKRTARRRYTRRKNRICYLQEIFS |
| | | NEMAKVDDSFFHRLEESFLVEEDKKHERHPIFGNIVDEVAYHEKYPT |
| | | IYHLRKKLVDSTDKADLRLIYLALAHMIKFRGHFLIEGDLNPDNSDV |
| | | DKLFIQLVQTYNQLFEENPINASGVDAKAILSARLSKSRRLENLIAQL |
| | | PGEKKNGLFGNLIALSLGLTPNFKSNFDLAEDAKLQLSKDTYDDDLD |
| | | NLLAQIGDQYADLFLAAKNLSDAILLSDILRVNTEITKAPLSASMIKR |
| | | YDEHHQDLTLLKALVRQQLPEKYKEIFFDQSKNGYAGYIDGGASQE |
| | | EFYKFIKPILEKMDGTEELLVKLNREDLLRKQRTFDNGSIPHQIHLGE |
| | | LHAILRRQEDFYPFLKDNREKIEKILTFRIPYYVGPLARGNSRFAWMT |
| | | RKSEETITPWNFEEVVDKGASAQSFIERMTNFDKNLPNEKVLPKHSL |
| | | LYEYFTVYNELTKVKYVTEGMRKPAFLSGEQKKAIVDLLFKTNRKV |
| | | TVKQLKEDYFKKIECFDSVEISGVEDRFNASLGTYHDLLKIIKDKDFL |
| | | DNEENEDILEDIVLTLTLFEDREMIEERLKTYAHLFDDKVMKQLKRR |
| | | RYTGWGRLSRKLINGIRDKQSGKTILDFLKSDGFANRNFMQLIHDDS |
| | | LTFKEDIQKAQVSGQGDSLHEHIANLAGSPAIKKGILQTVKVVDELV |
| | | KVMGRHKPENIVIEMARENQTTQKGQKNSRERMKRIEEGIKELGSQI |
| | | LKEHPVENTQLQNEKLYLYYLQNGRDMYVDQELDINRLSDYDVDAI |
| | | VPQSFLKDDSIDNKVLTRSDKNRGKSDNVPSEEVVKKMKNYWRQL |
| | | LNAKLITQRKFDNLTKAERGGLSELDKAGFIKRQLVETRQITKHVAQ |
| | | ILDSRMNTKYDENDKLIREVKVITLKSKLVSDFRKDFQFYKVREINN |
| | | YHHAHDAYLNAVVGTALIKKYPKLESEFVYGDYKVYDVRKMIAKS |
| | | EQEIGKATAKYFFYSNIMNFFKTEITLANGEIRKRPLIETNGETGEIVW |
| | | DKGRDFATVRKVLSMPQVNIVKKTEVQTGGFSKESILPKRNSDKLIA |
| | | RKKDWDPKKYGGFDSPTVAYSVLVVAKVEKGKSKKLKSVKELLGI |
| | | TIMERSSFEKNPIDFLEAKGYKEVKKDLIIKLPKYSLFELENGRKRML |
| | | ASAGELQKGNELALPSKYVNFLYLASHYEKLKGSPEDNEQKQLFVE |
| | | QHKHYLDEIIEQISEFSKRVILADANLDKVLSAYNKHRDKPIREQAEN |
| | | IIHLFTLTNLGAPAAFKYFDTTIDRKRYTSTKEVLDATLIHQSITGLYE |
| | | TRIDLSQLGGDPKKKRKV |
| 1157 | Zinc Finger | SRPGERPFQCRICMRNFSNNNNNNNHTRTHTGEKPFQCRICMRNFSN |
| | Array | NNNNNNHLRTH[linker]FQCRICMRNFSNNNNNNNHTRTHTGEKPFQ |
| | | CRICMRNFSNNNNNNNHLRTH[linker]FQCRICMRNFSNNNNNNNHT |
| | | RTHTGEKPFQCRICMRNFSNNNNNNNHLRTHLRGS |

1-134. (canceled)

135. An epigenetic editor comprising a fusion protein, wherein the fusion protein comprises:
(a) a first DNMT domain;
(b) a DNA binding domain; and
(c) a repressor domain,
wherein the repressor domain is selected from the group consisting of: ZIM3, ZNF436, ZNF257, ZNF675, ZNF490, ZNF320, ZNF331, ZNF816, ZNF680, ZNF41, ZNF189, ZNF528, ZNF543, ZNF554, ZNF140, ZNF610, ZNF264, ZNF350, ZNF8, ZNF582, ZNF30, ZNF324, ZNF98, ZNF669, ZNF677, ZNF596, ZNF214, ZNF37A, ZNF34, ZNF250, ZNF547, ZNF273, ZNF354A, ZFP82, ZNF224, ZNF33A, ZNF45, ZNF175, ZNF595, ZNF184, ZNF419, ZFP28-1, ZFP28-2, ZNF18, ZNF213, ZNF394, ZFP1, ZFP14, ZNF416, ZNF557, ZNF566, ZNF729, ZIM2, ZNF254, ZNF764, ZNF785, ZNF10, CBX5, RYBP, YAF2, MGA, CBX1, SCMH1, MPP8, SUMO3, HERC2, BIN1, PCGF2, TOX, FOXA1, FOXA2, IRF2BP1, IRF2BP2, IRF2BPL IRF-2BP1_2 N-terminal domain, HOXA13, HOXB13, HOXC13, HOXA11, HOXC11, HOXC10, HOXA10, HOXB9, HOXA9, ZFP28, ZN334, ZN568, ZN37A, ZN181, ZN510, ZN862, ZN140, ZN208, ZN248, ZN571, ZN699, ZN726, ZIK1, ZNF2, Z705F, ZNF14, ZN471, ZN624, ZNF84, ZNF7, ZN891, ZN337, Z705G, ZN529, ZN729, ZN419, Z705A, ZNF45, ZN302, ZN486, ZN621, ZN688, ZN33A, ZN554, ZN878, ZN772, ZN224, ZN184, ZN544, ZNF57, ZN283, ZN549, ZN211, ZN615, ZN253, ZN226, ZN730, Z585A, ZN732, ZN681, ZN667, ZN649, ZN470, ZN484, ZN431, ZN382, ZN254, ZN124, ZN607, ZN317, ZN620, ZN141, ZN584, ZN540, ZN75D, ZN555, ZN658, ZN684, RBAK, ZN829, ZN582, ZN112, ZN716, HKR1, ZN350, ZN480, ZN416, ZNF92, ZN100, ZN736, ZNF74, CBX1, ZN443, ZN195, ZN530, ZN782, ZN791, ZN331, Z354C, ZN157, ZN727, ZN550, ZN793, ZN235, ZNF8, ZN724, ZN573, ZN577, ZN789, ZN718, ZN300, ZN383, ZN429, ZN677, ZN850, ZN454, ZN257, ZN264, ZFP82, ZFP14, ZN485, ZN737, ZNF44, ZN596, ZN565, ZN543, ZFP69, SUMO1, ZNF12, ZN169, ZN433, SUMO3, ZNF98, ZN175, ZN347, ZNF25, ZN519, Z585B, ZIM3, ZN517, ZN846, ZN230, ZNF66, ZFP1, ZN713, ZN816, ZN426, ZN674, ZN627, ZNF20, Z587B, ZN316, ZN233, ZN611, ZN556, ZN234, ZN560, ZNF77, ZN682, ZN614, ZN785, ZN445, ZFP30, ZN225, ZN551, ZN610, ZN528, ZN284, ZN418, MPP8, ZN490, ZN805, Z780B, ZN763, ZN285, ZNF85, ZN223, ZNF90, ZN557, ZN425, ZN229, ZN606, ZN155, ZN222, ZN442, ZNF91, ZN135, ZN778, RYBP, ZN534, ZN586, ZN567, ZN440, ZN583, ZN441, ZNF43, CBX5, ZN589, ZNF10, ZN563, ZN561, ZN136, ZN630, ZN527, ZN333, Z324B, ZN786, ZN709, ZN792, ZN599, ZN613, ZF69B, ZN799, ZN569, ZN564, ZN546, ZFP92, YAF2, ZN723, ZNF34, ZN439, ZFP57, ZNF19, ZN404, ZN274, CBX3, ZNF30, ZN250, ZN570, ZN675, ZN695, ZN548, ZN132, ZN738, ZN420, ZN626, ZN559, ZN460, ZN268, ZN304, ZIM2, ZN605, ZN844, SUMO5, ZN101, ZN783, ZN417, ZN182, ZN823, ZN177, ZN197, ZN717, ZN669, ZN256, ZN251, CBX4, PCGF2, CDY2, CDYL2, HERC2, ZN562, ZN461, Z324A, ZN766, ID2, TOX, ZN274, SCMH1, ZN214, CBX7, ID1, CREM, SCX, ASCL1, ZN764, SCML2, TWST1, CREB1, TERF1, ID3, CBX8, CBX4, GSX1, NKX22, ATF1, TWST2, ZNF17, TOX3, TOX4, ZMYM3, I2BP1, RHXF1, SSX2, I2BPL, ZN680, CBX1, TRI68, HXA13, PHC3, TCF24, CBX3, HXB13, HEY1, PHC2, ZNF81, FIGLA, SAM11, KMT2B, HEY2, JDP2, HXC13, ASCL4, HHEX, HERC2, GSX2, BIN1, ETV7, ASCL3, PHC1, OTP, I2BP2, VGLL2, HXA11, PDLI4, ASCL2, CDX4, ZN860, LMBL4, PDIP3, NKX25, CEBPB, ISL1, CDX2, PROP1, SIN3B, SMBT1, HXC11, HXC10, PRS6A, VSX1, NKX23, MTG16, HMX3, HMX1, KIF22, CSTF2, CEBPE, DLX2, ZMYM3, PPARG, PRIC1, UNC4, BARX2, ALX3, TCF15, TERA, VSX2, HXD12, CDX1, TCF23, ALX1, HXA10, RX, CXXC5, SCML1, NFIL3, DLX6, MTG8, CBX8, CEBPD, SEC13, FIP1, ALX4, LHX3, PRIC2, MAGI3, NELL1, PRRX1, MTG8R, RAX2, DLX3, DLX1, NKX26, NAB1, SAMD7, PITX3, WDR5, MEOX2, NAB2, DHX8, FOXA2, CBX6, EMX2, CPSF6, HXC12, KDM4B, LMBL3, PHX2A, EMX1, NC2B, DLX4, SRY, ZN777, NELL1, ZN398, GATA3, BSH, SF3B4, TEAD1, TEAD3, RGAP1, PHF1, FOXA1, GATA2, FOXO3, ZN212, IRX4, ZBED6, LHX4, SIN3A, RBBP7, NKX61, TRI68, R51A1, MB3L1, DLX5, NOTC1, TERF2, ZN282, RGS12, ZN840, SPI2B, PAX7, NKX62, ASXL2, FOXO1, GATA3, GATA1, ZMYM5, ZN783, SPI2B, LRP1, MIXL1, SGT1, LMCD1, CEBPA, GATA2, SOX14, WTIP, PRP19, CBX6, NKX11, RBBP4, DMRT2, SMCA2 and fragments thereof.

136. The epigenetic editor of claim 135, wherein at least one of the repressor domains is selected from the group consisting of: SEQ ID NO: 67-595.

137. The epigenetic editor of claim 135, wherein the DNA binding domain binds to a target sequence in a target chromosome comprising a target gene.

138. The epigenetic editor of claim 135, wherein the repressor domain specifically binds to an epigenetic effector protein in a cell comprising a target gene and directs the epigenetic editor to the target gene to effect an epigenetic modification in a nucleotide in the target gene or a histone bound to the target gene.

139. The epigenetic editor of claim 135, wherein the repressor domains is selected from the group consisting of: ZIM3, ZNF264, ZN577, ZN793, ZFP28, ZN627, RYBP, TOX, TOX3, TOX4, I2BP1, SCMH1, SCML2, CDYL2, CBX8, CBX5, and CBX1, and fragments thereof.

140. The epigenetic editor of claim 135, wherein the fusion protein further comprises a second DNMT domain.

141. The epigenetic editor of claim 135, wherein the first DNMT domain is selected from the group consisting of a DNMT3A domain, a DNMT3B domain, a DNMT3C domain, and a DNMT3L domain.

142. The epigenetic editor of claim 135, wherein the first DNMT domain is a human DNMT3A domain or a human DNMT3L domain.

143. The epigenetic editor of claim 142, wherein the first DNMT domain is a DNMT3A domain and the second DNMT domain is a DNMT3L domain, or a catalytic portion thereof.

144. The epigenetic editor of claim 135, wherein the first DNMT domain and the second DNMT domain are selected from the group consisting of SEQ ID NO: 32-66.

145. The epigenetic editor of claim 135, wherein the DNA binding domain comprises a zinc finger motif or a zinc finger array.

146. The epigenetic editor of claim 135, wherein the DNA binding domain comprises a nucleic acid guided DNA binding domain bound to a guide polynucleotide.

147. The epigenetic editor of claim 146, wherein the DNA binding domain comprises CRISPR-Cas protein bound to the guide polynucleotide.

148. The epigenetic editor of claim 146, wherein the guide polynucleotide hybridizes with a target sequence.

149. The epigenetic editor of claim 147, wherein the CRISPR-Cas protein comprises a nuclease inactive Cas9 (dCas9).

150. The epigenetic editor of claim 149, wherein the dCas9 is a dSpCas9.

151. The epigenetic editor of claim 150, wherein the dSpCas9 is defined as SEQ ID NO: 3.

152. The epigenetic editor of claim 135, wherein the fusion protein domain comprises from N-terminus to C-terminus DNMT3A-DNMT3L-dSpCas9—the repressor domain.

153. A method of modifying an epigenetic state of a target gene in a target chromosome, the method comprising contacting the target chromosome with an epigenetic editor, wherein the epigenetic editor comprises:
(a) a first DNMT domain;
(b) a DNA binding domain; and
(c) a repressor domain,
wherein the repressor domain is selected from the group consisting of: ZIM3, ZNF436, ZNF257, ZNF675, ZNF490, ZNF320, ZNF331, ZNF816, ZNF680, ZNF41, ZNF189, ZNF528, ZNF543, ZNF554, ZNF140, ZNF610, ZNF264, ZNF350, ZNF8, ZNF582, ZNF30, ZNF324, ZNF98, ZNF669, ZNF677, ZNF596, ZNF214, ZNF37A, ZNF34, ZNF250, ZNF547, ZNF273, ZNF354A, ZFP82, ZNF224, ZNF33A, ZNF45, ZNF175, ZNF595, ZNF184, ZNF419, ZFP28-1, ZFP28-2, ZNF18, ZNF213, ZNF394, ZFP1, ZFP14, ZNF416, ZNF557, ZNF566, ZNF729, ZIM2, ZNF254, ZNF764, ZNF785, ZNF10, CBX5, RYBP, YAF2, MGA, CBX1, SCMH1, MPP8, SUMO3, HERC2, BIN1, PCGF2, TOX, FOXA1, FOXA2, IRF2BP1, IRF2BP2, IRF2BPL IRF-2BP1_2 N-terminal domain, HOXA13, HOXB13, HOXC13, HOXA11, HOXC11, HOXC10, HOXA10, HOXB9, HOXA9, ZFP28, ZN334, ZN568, ZN37A, ZN181, ZN510, ZN862, ZN140, ZN208, ZN248, ZN571, ZN699, ZN726, ZIK1, ZNF2, Z705F, ZNF14, ZN471, ZN624, ZNF84, ZNF7, ZN891, ZN337, Z705G, ZN529, ZN729, ZN419, Z705A, ZNF45, ZN302, ZN486, ZN621, ZN688, ZN33A, ZN554, ZN878, ZN772, ZN224, ZN184, ZN544, ZNF57, ZN283, ZN549, ZN211, ZN615, ZN253, ZN226, ZN730, Z585A, ZN732, ZN681, ZN667, ZN649, ZN470, ZN484, ZN431, ZN382, ZN254, ZN124, ZN607, ZN317, ZN620, ZN141, ZN584, ZN540, ZN75D, ZN555, ZN658, ZN684, RBAK, ZN829, ZN582, ZN112, ZN716, HKR1, ZN350, ZN480, ZN416, ZNF92, ZN100, ZN736, ZNF74, CBX1, ZN443, ZN195, ZN530, ZN782, ZN791, ZN331, Z354C, ZN157, ZN727, ZN550, ZN793, ZN235, ZNF8, ZN724, ZN573, ZN577, ZN789, ZN718, ZN300, ZN383, ZN429, ZN677, ZN850, ZN454, ZN257, ZN264, ZFP82, ZFP14, ZN485, ZN737, ZNF44, ZN596, ZN565, ZN543, ZFP69, SUMO1, ZNF12, ZN169, ZN433, SUMO3, ZNF98, ZN175, ZN347, ZNF25, ZN519, Z585B, ZIM3, ZN517, ZN846, ZN230, ZNF66, ZFP1, ZN713, ZN816, ZN426, ZN674, ZN627, ZNF20, Z587B, ZN316, ZN233, ZN611, ZN556, ZN234, ZN560, ZNF77, ZN682, ZN614, ZN785, ZN445, ZFP30, ZN225, ZN551, ZN610, ZN528, ZN284, ZN418, MPP8, ZN490, ZN805, Z780B, ZN763, ZN285, ZNF85, ZN223, ZNF90, ZN557, ZN425, ZN229, ZN606, ZN155, ZN222, ZN442, ZNF91, ZN135, ZN778, RYBP, ZN534, ZN586, ZN567, ZN440, ZN583, ZN441, ZNF43, CBX5, ZN589, ZNF10, ZN563, ZN561, ZN136, ZN630, ZN527, ZN333, Z324B, ZN786, ZN709, ZN792, ZN599, ZN613, ZF69B, ZN799, ZN569, ZN564, ZN546, ZFP92, YAF2, ZN723, ZNF34, ZN439, ZFP57, ZNF19, ZN404, ZN274, CBX3, ZNF30, ZN250, ZN570, ZN675, ZN695, ZN548, ZN132, ZN738, ZN420, ZN626, ZN559, ZN460, ZN268, ZN304, ZIM2, ZN605, ZN844, SUMO5, ZN101, ZN783, ZN417, ZN182, ZN823, ZN177, ZN197, ZN717, ZN669, ZN256, ZN251, CBX4, PCGF2, CDY2, CDYL2, HERC2, ZN562, ZN461, Z324A, ZN766, ID2, TOX, ZN274, SCMH1, ZN214, CBX7, ID1, CREM, SCX, ASCL1, ZN764, SCML2, TWST1, CREB1, TERF1, ID3, CBX8, CBX4, GSX1, NKX22, ATF1, TWST2, ZNF17, TOX3, TOX4, ZMYM3, I2BP1, RHXF1, SSX2, I2BPL, ZN680, CBX1, TRI68, HXA13, PHC3, TCF24, CBX3, HXB13, HEY1, PHC2, ZNF81, FIGLA, SAM11, KMT2B, HEY2, JDP2, HXC13, ASCL4, HHEX, HERC2, GSX2, BIN1, ETV7, ASCL3, PHC1, OTP, I2BP2, VGLL2, HXA11, PDLI4, ASCL2, CDX4, ZN860, LMBL4, PDIP3, NKX25, CEBPB, ISL1, CDX2, PROP1, SIN3B, SMBT1, HXC11, HXC10, PRS6A, VSX1, NKX23, MTG16, HMX3, HMX1, KIF22, CSTF2, CEBPE, DLX2, ZMYM3, PPARG, PRIC1, UNC4, BARX2, ALX3, TCF15, TERA, VSX2, HXD12, CDX1, TCF23, ALX1, HXA10, RX, CXXC5, SCML1, NFIL3, DLX6, MTG8, CBX8, CEBPD, SEC13, FIP1, ALX4, LHX3, PRIC2, MAGI3, NELL1, PRRX1, MTG8R, RAX2, DLX3, DLX1, NKX26, NAB1, SAMD7, PITX3, WDR5, MEOX2, NAB2, DHX8, FOXA2, CBX6, EMX2, CPSF6, HXC12, KDM4B, LMBL3, PHX2A, EMX1, NC2B, DLX4, SRY, ZN777, NELL1, ZN398, GATA3, BSH, SF3B4, TEAD1, TEAD3, RGAP1, PHF1, FOXA1, GATA2, FOXO3, ZN212, IRX4, ZBED6, LHX4, SIN3A, RBBP7, NKX61, TRI68, R51A1, MB3L1, DLX5, NOTC1, TERF2, ZN282, RGS12, ZN840, SPI2B, PAX7, NKX62, ASXL2, FOXO1, GATA3, GATA1, ZMYM5, ZN783, SPI2B, LRP1, MIXL1, SGT1, LMCD1, CEBPA, GATA2, SOX14, WTIP, PRP19, CBX6, NKX11, RBBP4, DMRT2, SMCA2 and fragments thereof.

154. A method of treating a disease in a subject in need thereof, the method comprising administering to the subject an epigenetic editor, wherein the epigenetic editor comprises:
(a) a first DNMT domain;
(b) a DNA binding domain; and
(c) a repressor domain,
wherein the repressor domain is selected from the group consisting of: ZIM3, ZNF436, ZNF257, ZNF675, ZNF490, ZNF320, ZNF331, ZNF816, ZNF680, ZNF41, ZNF189, ZNF528, ZNF543, ZNF554, ZNF140, ZNF610, ZNF264, ZNF350, ZNF8, ZNF582, ZNF30, ZNF324, ZNF98, ZNF669, ZNF677, ZNF596, ZNF214, ZNF37A, ZNF34, ZNF250, ZNF547, ZNF273, ZNF354A, ZFP82, ZNF224, ZNF33A, ZNF45, ZNF175, ZNF595, ZNF184, ZNF419, ZFP28-1, ZFP28-2, ZNF18, ZNF213, ZNF394, ZFP1, ZFP14, ZNF416, ZNF557, ZNF566, ZNF729, ZIM2, ZNF254, ZNF764, ZNF785, ZNF10, CBX5, RYBP, YAF2, MGA, CBX1, SCMH1, MPP8, SUMO3, HERC2, BIN1, PCGF2, TOX, FOXA1, FOXA2, IRF2BP1, IRF2BP2, IRF2BPL IRF-2BP1_2 N-terminal domain, HOXA13, HOXB13, HOXC13, HOXA11, HOXC11, HOXC10, HOXA10, HOXB9, HOXA9, ZFP28, ZN334, ZN568, ZN37A, ZN181, ZN510, ZN862, ZN140, ZN208, ZN248, ZN571, ZN699, ZN726, ZIK1, ZNF2, Z705F, ZNF14, ZN471, ZN624, ZNF84, ZNF7, ZN891, ZN337, Z705G, ZN529, ZN729, ZN419, Z705A, ZNF45, ZN302, ZN486, ZN621, ZN688, ZN33A, ZN554, ZN878, ZN772, ZN224, ZN184, ZN544, ZNF57, ZN283, ZN549, ZN211, ZN615, ZN253, ZN226, ZN730, Z585A, ZN732, ZN681, ZN667, ZN649, ZN470, ZN484, ZN431, ZN382, ZN254, ZN124, ZN607, ZN317, ZN620, ZN141, ZN584, ZN540, ZN75D, ZN555, ZN658, ZN684, RBAK, ZN829, ZN582, ZN112, ZN716, HKR1, ZN350, ZN480, ZN416, ZNF92, ZN100, ZN736, ZNF74, CBX1, ZN443, ZN195, ZN530, ZN782, ZN791, ZN331, Z354C, ZN157, ZN727, ZN550, ZN793, ZN235, ZNF8, ZN724, ZN573, ZN577, ZN789, ZN718, ZN300, ZN383, ZN429, ZN677, ZN850, ZN454, ZN257, ZN264, ZFP82, ZFP14, ZN485, ZN737, ZNF44, ZN596, ZN565, ZN543, ZFP69, SUMO1, ZNF12, ZN169, ZN433, SUMO3, ZNF98, ZN175, ZN347, ZNF25, ZN519, Z585B, ZIM3, ZN517, ZN846, ZN230, ZNF66, ZFP1, ZN713, ZN816, ZN426, ZN674, ZN627, ZNF20, Z587B, ZN316, ZN233, ZN611, ZN556, ZN234, ZN560, ZNF77, ZN682, ZN614, ZN785, ZN445, ZFP30, ZN225, ZN551, ZN610, ZN528, ZN284, ZN418, MPP8, ZN490, ZN805, Z780B, ZN763, ZN285, ZNF85, ZN223, ZNF90, ZN557, ZN425, ZN229, ZN606, ZN155, ZN222, ZN442, ZNF91, ZN135, ZN778, RYBP, ZN534, ZN586, ZN567, ZN440, ZN583, ZN441, ZNF43, CBX5, ZN589, ZNF10, ZN563, ZN561, ZN136, ZN630, ZN527, ZN333, Z324B, ZN786, ZN709, ZN792, ZN599, ZN613, ZF69B, ZN799, ZN569, ZN564, ZN546, ZFP92, YAF2, ZN723, ZNF34, ZN439, ZFP57, ZNF19, ZN404, ZN274, CBX3, ZNF30, ZN250, ZN570, ZN675, ZN695, ZN548, ZN132, ZN738, ZN420, ZN626, ZN559, ZN460, ZN268, ZN304, ZIM2, ZN605, ZN844, SUMO5, ZN101, ZN783, ZN417, ZN182, ZN823, ZN177, ZN197, ZN717, ZN669, ZN256, ZN251, CBX4, PCGF2, CDY2, CDYL2, HERC2, ZN562, ZN461, Z324A, ZN766, ID2, TOX, ZN274, SCMH1, ZN214, CBX7, ID1, CREM, SCX, ASCL1, ZN764, SCML2, TWST1, CREB1, TERF1, ID3, CBX8, CBX4, GSX1, NKX22, ATF1, TWST2, ZNF17, TOX3, TOX4, ZMYM3, I2BP1, RHXF1, SSX2, I2BPL, ZN680, CBX1, TRI68, HXA13, PHC3, TCF24, CBX3, HXB13, HEY1, PHC2, ZNF81, FIGLA, SAM11, KMT2B, HEY2, JDP2, HXC13, ASCL4, HHEX, HERC2, GSX2, BIN1, ETV7, ASCL3, PHC1, OTP, I2BP2, VGLL2, HXA11, PDLI4, ASCL2, CDX4, ZN860, LMBL4, PDIP3, NKX25, CEBPB, ISL1, CDX2, PROP1, SIN3B, SMBT1, HXC11, HXC10, PRS6A, VSX1, NKX23, MTG16, HMX3, HMX1, KIF22, CSTF2, CEBPE, DLX2, ZMYM3, PPARG, PRIC1, UNC4, BARX2, ALX3, TCF15, TERA, VSX2, HXD12, CDX1, TCF23, ALX1, HXA10, RX, CXXC5, SCML1, NFIL3, DLX6, MTG8, CBX8, CEBPD, SEC13, FIP1, ALX4, LHX3, PRIC2, MAGI3, NELL1, PRRX1, MTG8R, RAX2, DLX3, DLX1, NKX26, NAB1, SAMD7, PITX3, WDR5, MEOX2, NAB2, DHX8, FOXA2, CBX6, EMX2, CPSF6, HXC12, KDM4B, LMBL3, PHX2A, EMX1, NC2B, DLX4, SRY, ZN777, NELL1, ZN398, GATA3, BSH, SF3B4, TEAD1, TEAD3, RGAP1, PHF1, FOXA1, GATA2, FOXO3, ZN212, IRX4, ZBED6, LHX4, SIN3A, RBBP7, NKX61, TRI68, R51A1, MB3L1, DLX5, NOTC1, TERF2, ZN282, RGS12, ZN840, SPI2B, PAX7, NKX62, ASXL2, FOXO1, GATA3, GATA1, ZMYM5, ZN783, SPI2B, LRP1, MIXL1, SGT1, LMCD1, CEBPA, GATA2, SOX14, WTIP, PRP19, CBX6, NKX11, RBBP4, DMRT2, SMCA2 and fragments thereof.
